(19) United States
(12) Patent Application Publication
(10) Pub. No.: US 2023/0272387 A1
(43) Pub. Date: Aug. 31, 2023

(54) COMPOSITIONS AND METHODS FOR TREATING DISORDERS ASSOCIATED WITH LOSS-OF-FUNCTION MUTATIONS IN SCN2A

(71) Applicant: The Florey Institute of Neuroscience and Mental Health, Melbourne (AU)

(72) Inventor: Steven Petrou, Eltham (AU)

(21) Appl. No.: 18/005,800

(22) PCT Filed: Jul. 22, 2021
(86) PCT No.: PCT/AU2021/050788

§ 371 Date: Jan. 17, 2023

(30) Foreign Application Priority Data
Jul. 22, 2020 (AU) 2020902550

(51) Int. Cl. C12N 15/113

(52) U.S. Cl. CPC C12N 15/113; C12N 2310/11; C12N 2320/33; C12N 2310/315

(57) ABSTRACT

Provided herein are methods for increasing levels of SCN2A protein in a cell, comprising contacting the cell with an antisense oligonucleotide that enhances splicing at a splice site of a retained intron in an intron-retaining SCN2A mRNA or pre-mRNA, wherein the retained intron is selected from among intron 1, 2, 3, 4, 5, 11, 13, 17 and 24 and wherein the antisense oligonucleotide comprises a sequence of nucleobases that is complementary to a target region in the SCN2A mRNA or pre-mRNA. Also provided are antisense oligonucleotides for use in such methods. Also provided are methods for treating disorders associated with a heterozygous loss-of-function mutation in SCN2A, comprising administering to the subject such antisense oligonucleotides.

### FIELD OF THE DISCLOSURE

The present disclosure relates generally to compositions and methods suitable for treating a disorder associated with loss-of-function mutations in SCN2A. More specifically, the disclosure relates to antisense oligonucleotides specific for SCN2A and their use for treating a disorder associated with heterozygous loss-of-function mutations of SCN2A.

### BACKGROUND TO THE DISCLOSURE

The SCN2A protein (also referred to as sodium voltage-gated channel alpha subunit 2, brain Nav1.2 voltage-gated sodium channel, SCN2A1, SCN2A2, Nav1.2, HBA, NAC2, BFIC3, BFIS3, BFNIS, HBSCI, EIEE11 or HBSCII,) is encoded by the SCN2A gene on human chromosome 2 at 2q24.3 (HGNC: 10588; NCBI gene: 6326; NCBI Reference Sequence: NG_008143.1). SCN2A plays a key role in the initiation and propagation of action potentials and the modulation of neuronal and brain excitability. SCN2A expression is detected in brain and kidney, however its expression is predominantly in the brain.

In the brain SCN2A is heterogeneously expressed, and mutations, dysfunction, and/or dysregulation of the protein or levels of functional protein are associated with various neurodevelopmental disorders. With the large sequencing studies performed in the recent years, SCN2A has emerged as the major single gene cause of neurogenetic disorders. SCN2A mutations are the second most common cause of severe genetic epilepsy (including developmental and epileptic encephalocopathies (DEE)) in the human population (Nakamura et al, 2013; Howell et al, 2015), and are also frequently associated with intellectual disability (Rauch et al, 2012; Li et al, 2016), autism spectrum disorders (Iossifov et al, 2014; Sanders et al, 2015; Wang et al, 2016) and schizophrenia (Carroll et al, 2016).

The SCN2A primary transcript is alternatively spliced, resulting in multiple mRNA transcript variants, some of which are known to be developmentally regulated. Some forms of epileptic encephalopathies have been associated with variants in SCN2A. Loss-of-function mutations or alterations (e.g. nonsense mutations, large deletion and frameshift mutations) in SCN2A may result in a decreased level of functional protein, or the formation of a truncated transcript, leading to the haploinsufficiency. The SCN2A gene may have a missense or nonsense mutation in one or both alleles. In some instances, expression of SCN2A may be dysregulated by a primary disorder or a mutation in the gene or a regulatory region, thus resulting in decreased levels of protein or functional protein. In other instances, SCN2A levels are not altered, but increasing levels of functional SCN2A protein provides a therapeutic effect for a neurological disorder or psychiatric disorder.

The only therapies currently available to patients with SCN2A or any other disorder associated with a heterozygous loss-of-function mutation in SCN2A are those that treat a symptom of the disorder, such as agents to treat epileptic seizures or interventions (e.g. speech therapy, physiotherapy, occupational therapy, etc.) to treat the behavioural or developmental symptoms or intellectual disability. Consequently, there remains a need for agents, compositions and methods for the treatment of MRD5 or any other disorder associated with a heterozygous loss-of-function mutation in SCN2A.

### SUMMARY OF THE DISCLOSURE

The present disclosure is predicated, at least in part, on the determination that a number of introns are retained in mature SCN2A mRNA in brain tissue, including introns 1, 2, 3, 4, 5, 11, 13, 17 and 24. Introns 2 and 17 in particular have relatively high retention rates.

Intron retention is a form of gene regulation that serves to direct intron-harbouring transcripts to nonsense-mediated decay, thereby reducing gene expression (Kurosaki & Maquat, 2016, J Cell Sci. 129 (3): 461-467). Intron-retaining transcripts have also been shown to serve as a reservoir of RNAs that undergo splicing and translation whenever their expression is required (Jacob & Smith, 2017, Hum Genet. 136 (9): 1043-1057). The process of transcription, which occurs at a rate of 1-4 kb/min, is especially rate-limiting for neuronal activation following neuronal stimuli (Darzarcq et al, 2007, Nat Strut. Mol. Biol. 14, 796-806). In contrast, splicing of retained introns is a much faster process, taking just seconds to a few minutes (Bayer and Osheim, 1988, Genes Dev. 2, 754-765; Singh and Padgett, 2009, Nat. Strut. Mol. Biol. 16, 1128-1133). Consequently, neurons can achieve a faster mode of gene regulation using intron retention and subsequent splicing and translation as compared to de novo transcription and translation. Intron retention has been demonstrated to occur in a pool of polyadenylated transcripts that are retained in the nucleus. Following neuronal stimulation, they undergo intron excision and are transported to the cytoplasm for further processing, thereby aiding in faster gene regulation.

As demonstrated herein, a retained intron in SCN2A mRNA or pre-RNA (such as polyadenylated SCN2A mRNA or pre-mRNA transcripts in the nucleus of a cell) can be targeted with antisense oligonucleotides so as to enhance splicing at the splice site of the retained intron, resulting in an increase in the amount of fully-spliced SCN2A mRNA. Consequently, the antisense oligonucleotides provided herein are useful for increasing the amount of SCN2A produced by a cell. The antisense oligonucleotides provided herein are therefore also useful as therapeutic agents for the treatment of diseases or disorders associated with heterozygous loss-of-function mutations in SCN2A, such as severe genetic epilepsy (including developmental and epileptic encephalocopathies (DEE)) in the human population, and it is also frequently associated with intellectual disability, autism spectrum disorders and schizophrenia, wherein increasing the levels of SCN2A protein can provide a therapeutic effect.

Accordingly, in one aspect, provided is a method for increasing levels of SCN2A protein in a cell, comprising contacting the cell with an antisense oligonucleotide that enhances splicing at a splice site of a retained intron in an intron-retaining SCN2A mRNA or pre-mRNA, wherein the retained intron is selected from among introns 1, 2, 3, 4, 5, 11, 13, 17 and 24 and wherein the antisense oligonucleotide comprises a sequence of nucleobases that is complementary to a target region in the SCN2A mRNA or pre-mRNA.

In an embodiment, the retained intron is intron 2, comprising the nucleotide sequence set forth in SEQ ID NO: 12.

In another aspect, provided is a method for increasing levels of SCN2A protein in a subject, comprising administering to the subject an antisense oligonucleotide that enhances splicing at a splice site of a retained intron in an intron-retaining SCN2A mRNA or pre-mRNA, wherein the retained intron is selected from among intron 1, 2, 3, 4, 5, 11, 13, 17 and 24 and wherein the antisense oligonucleotide comprises a sequence of nucleobases that is complementary to a target region in the SCN2A mRNA or pre-mRNA. In some embodiments, the subject has a heterozygous loss-of-function mutation in SCN2A.

In some embodiments, the subject has a disorder associated with a heterozygous loss-of-function mutation in SCN2A, such as genetic epilepsy (including developmental and epileptic encephalocopathies (DEE)), intellectual disability, autism spectrum disorders and schizophrenia.

Also provided is a method for treating a disorder associated with a heterozygous loss-of-function mutation in SCN2A, comprising administering to the subject an antisense oligonucleotide that enhances splicing at a splice site of a retained intron in an intron-retaining SCN2A mRNA or pre-mRNA, wherein the retained intron is selected from among introns 1, 2, 3, 4, 5, 11, 13, 17 and 24, and wherein the antisense oligonucleotide comprises a sequence of nucleobases that is complementary to a target region in the SCN2A mRNA or pre-mRNA. In particular examples, genetic epilepsy (including developmental and epileptic encephalocopathies (DEE)), intellectual disability, autism spectrum disorders and schizophrenia.

In some embodiments of the methods of the disclosure, the antisense oligonucleotide binds to, or adjacent to, an intron splicing silencer (ISS); binds to nucleotides within a G-quadruplex; or binds to nucleotides with an RNA secondary structure. The ISS may be recognised by a heterogeneous nuclear ribonucleoprotein (hnRNP), such as hnRNPA1 or hnRNP I.

In one example, the retained intron is intron 2 and the ISS is at positions +8-+12, +33-+36 or +60-+65, relative to the 5′ splice site of intron 2. Optionally, the ISS is at positions+8-+12, +33-+36 or +60-+65 relative to the first nucleotide of SEQ ID NO:12.

In particular embodiments, the retained intron is intron 2 and the target region spans positions −6-+12, −5-+13, −4-+14, −3-+15, −2-+16, −1-+19, 0-+18, +1-+19, +2-+20, +3-21, +4-22, +5-+23, +6-+24, +7-+25, +8-26, +9-+27, +10-+28, +11-+29, +12-+30, +13-+31, +14-+32, +15-+33, +16-+34, +17-+35, +18-+36, +19-+37, +20-+38, +21-+39, +22-+40, +23-+41, +24-+42, +25-+43, +26-+44, +27-+45, +28-+46, +29-+47, +30-+48, +31-+49, +32-+50, +33-+51, +34-+52, +35-+53, +36-+54, +37-+55, +38-+56, +39-+57, +40-+58, +41-+59, +42-+60, +43-+61, +44-+62, +45-+63, +46-+64, +47-+65, +48-+66, +49-+67, +50-+68, +51-+69, +52-+70, +53-+71, +54-+72, +55-+73, +56-+74, +57-+75, +58-+76, +59-+77, +60-+78, +61-+79, +62-+80, +63-+81, +64-+82, +65-+83, +66-+84, +67-+85, +68-+86, +69-+87, +70-+88, +71-+89, +72-+90, +73-+91, +74-+92, +75-+93, +76-+94, +77-+95, +78-+96, +79-+97, +80-+98, +81-+99, relative to the 5′ splice site of intron 2, optionally relative to the first nucleotide of SEQ ID NO:12. In some embodiments, the antisense oligonucleotide comprises a sequence having at least or about 70%, 80%, or 90% sequence identity to a sequence set forth in any one of SEQ ID NOs:115-142, or a sequence having at least 8, 9, 10, 11, 12, 13, 14 or 15 contiguous nucleotides from a sequence set forth in any one of SEQ ID NOs: 115-142. In one embodiment, the antisense oligonucleotide comprises the sequence set forth in SEQ ID NO:126 or SEQ ID NO:138, or a sequence comprising at least 8, 9, 10, 11, 12, 13, 14 or 15 contiguous nucleotides from a sequence set forth in SEQ ID NO:126 or SEQ ID NO:138.

In further embodiments, the retained intron is intron 2 and the target region spans positions −19-−1, −20-−2, −21-−3, −22-−4, −23-−5, −24-−6, −25-−7, −26-−8, −27-−9, −28-−10, −29-−11, −30-−12, −31-−13, −32-−14, −33-−15, −38-−20, −39-−21, −48-−30, −49-−31, −51-−33, −52-−34, −53-−35, −54-−36, −62-−44, −64-−46, −65-−47, −66-−48, −67-−49, −76-−58, −77-−59, −78-−60, −79-−61, −82-−64, −83-65, −84-−66, −85-−67, −86-−68, −87-−69, −90-−72, −91-−73, −92-−74, −95-−77, −97-−79, −98-−80, −99-−81, −101-−83, −102-−84, −103-−85, −104-−86, −105-−87, −106-−88, −107-−89, −108-−90, −109-−91, −110-−92, −111-−93, −112-−94, −113-−95, −114-−96, −115-−97, −116-−98, −117-−99, relative to the 3′ splice site of intron 2, optionally relative to the last nucleotide of SEQ ID NO:12. In some embodiments, the antisense oligonucleotide comprises a sequence having at least or about 70%, 80%, or 90% sequence identity to a sequence set forth in any one of SEQ ID NOs:143-205, or a sequence having at least 8, 9, 10, 11, 12, 13, 14 or 15 contiguous nucleotides from a sequence set forth in any one of SEQ ID NOs: 143-205. In one embodiment, the antisense oligonucleotide comprises the sequence set forth in SEQ ID NO:155, or a sequence comprising at least 8, 9, 10, 11, 12, 13, 14 or 15 contiguous nucleotides from a sequence set forth in SEQ ID NO:155.

In the methods of the present disclosure, the antisense oligonucleotide may consist of, for example, from 8 to 50, 8 to 40, 8 to 35, 8 to 30, 8 to 25, 8 to 20, 8 to 15, 9 to 50, 9 to 40, 9 to 35, 9 to 30, 9 to 25, 9 to 20, 9 to 15, 10 to 50, 10 to 40, 10 to 35, 10 to 30, 10 to 25, 10 to 20, 10 to 15, 11 to 50, 11 to 40, 11 to 35, 11 to 30, 11 to 25, 11 to 20, 11 to 15, 12 to 50, 12 to 40, 12 to 35, 12 to 30, 12 to 25, 12 to 20, or 12 to 15 nucleobases. In some embodiments, the antisense oligonucleotide is at least 70%, 80%, 85%, 86%, 87%, 88%, 89%, 90%, 91%, 92%, 93%, 94%, 95%, 96%, 97%, 98%, or 99% complementary to the target region. In particular embodiments, the antisense oligonucleotide comprises least 8, 9, 10, 11, 12, 13, 14, 15, 16, 17, 18, 19 or 20 contiguous nucleobases that are 100% complementary to the target region.

The antisense oligonucleotide utilised in the methods of the present disclosure may comprise at least one modification, e.g. a nucleobase modification, a modification of the oligonucleotide backbone or a modification of a ribose sugar. In one embodiment, the antisense oligonucleotide comprises a modified sugar selected from among a 2′-O-methyl (2OMe), 2′-O-methoxy-ethyl (MOE), locked nucleic acids (LNA), 2′-fluoro or S-constrained-ethyl (cEt). In a further embodiment, the antisense oligonucleotide comprises backbone that comprises phosphorothioates.

In the methods of the disclosure that include administering to the subject an antisense oligonucleotide, the subject may first be determined to have a loss-of-function mutation in SCN2A. In particular embodiments, the subject has been genotyped to identify a heterozygous loss-of-function mutation in SCN2A. The antisense oligonucleotide may be administered to the subject by parenteral administration (e.g. subcutaneous administration, intravenous administration, intramuscular administration, intraarterial administration, intraperitoneal administration, or intracranial administration) or intranasal administration (e.g. intrathecal or intracerebroventricular administration). In some embodiments, the antisense oligonucleotide or composition is administered to the subject about every 1, 2, 3, 4, 5, 6, 7, 8, 9, 10, 11, 12 or more months.

In a further aspect, provided herein is an antisense oligonucleotide comprising a sequence of nucleobases that is complementary to a target region in an intron-retaining SCN2A mRNA or pre-mRNA, wherein the target region is in a retained intron and wherein the retained intron is selected from among intron 1, 2, 3, 4, 5, 11, 13, 17 and 24.

In some embodiments, the antisense oligonucleotide binds to, or adjacent to, an intron splicing silencer (ISS); binds to nucleotides within a G-quadruplex; or binds to nucleotides with an RNA secondary structure. In a particular embodiment, the ISS is recognised by a heterogeneous nuclear ribonucleoproteins (hnRNP), e.g. hnRNPA1 or hnRNP I.

In one embodiment, the retained intron in which the target region is present is intron 2 and the ISS is at positions+8-+12, +33-+36 or +60-+65 relative to the 5′ splice site of intron 2. Optionally, the ISS is at positions+8-+12, +33-+36 or +60-+65 relative to the first nucleotide of SEQ ID NO:12.

In particular embodiments, the retained intron is intron 2 and the target region spans positions −4-+14, −3-+15, −2-+16, +6-+24, +7-+25, +8-26, +9-+27, +10-+28, +14-+32, +19-+37, +20-+38, +21-+39, +22-+40, +23-+41, +24-+42, +25-+43, +26-+44, +27-+45, +28-+46, +29-+47, +30-+48, +31-+49, +32-+50, +33-+51, +34-+52, +35-+53, +43-+61, +45-+63, +46-+64, +49-+67, +52-+70, +59-+77, +64-+82, +65-+83, +68-+86, +69-+87, +70-+88, +71-+89, +72-+90 and +73-+91, relative to the 5′ splice site of intron 2, optionally relative to the first nucleotide of SEQ ID NO:12. In some embodiments, the antisense oligonucleotide comprises a sequence having at least or about 70%, 80%, or 90% sequence identity to a sequence set forth in any one of SEQ ID NOs:115-142, or a sequence having at least 8, 9, 10, 11, 12, 13, 14 or 15 contiguous nucleotides from a sequence set forth in any one of SEQ ID NOs: 115-142. In one embodiment, the antisense oligonucleotide comprises the sequence set forth in SEQ ID NO:126 or SEQ ID NO:138, or a sequence comprising at least 8, 9, 10, 11, 12, 13, 14 or 15 contiguous nucleotides from a sequence set forth in SEQ ID NO:126 or SEQ ID NO:138.

In further embodiments, the retained intron is intron 2 and the target region spans positions −19-−1, −21-−3, −30-−12, −31-−13, −32-−14, −62-−44, −64-−46, −67-−49, −76-−58, −77-−59, −78-−60, −79-−61 −82-−64, −83-65, −84-−66, −85-−67, −86-−68, −87-−69, −95-−77, −97-−79, −100-−82, −101-−83, −102-−84, −103-−85, −107-−89, −109-−91, −111-−93, −113-−95, and −114-−96, relative to the 3′ splice site of intron 2, optionally relative to the last nucleotide of SEQ ID NO:12. In some embodiments, the antisense oligonucleotide comprises a sequence having at least or about 70%, 80%, or 90% sequence identity to a sequence set forth in any one of SEQ ID NOs:143-205, or a sequence having at least 8, 9, 10, 11, 12, 13, 14 or 15 contiguous nucleotides from a sequence set forth in any one of SEQ ID NOs: 143-205. In one embodiment, the antisense oligonucleotide comprises the sequence set forth in SEQ ID NO:155, or a sequence comprising at least 8, 9, 10, 11, 12, 13, 14 or 15 contiguous nucleotides from a sequence set forth in SEQ ID NO:155.

In some embodiments, the antisense oligonucleotide may consist of, for example, from 8 to 50, 8 to 40, 8 to 35, 8 to 30, 8 to 25, 8 to 20, 8 to 15, 9 to 50, 9 to 40, 9 to 35, 9 to 30, 9 to 25, 9 to 20, 9 to 15, 10 to 50, 10 to 40, 10 to 35, 10 to 30, 10 to 25, 10 to 20, 10 to 15, 11 to 50, 11 to 40, 11 to 35, 11 to 30, 11 to 25, 11 to 20, 11 to 15, 12 to 50, 12 to 40, 12 to 35, 12 to 30, 12 to 25, 12 to 20, or 12 to 15 nucleobases. In some embodiments, the antisense oligonucleotide is at least 70%, 80%, 85%, 86%, 87%, 88%, 89%, 90%, 91%, 92%, 93%, 94%, 95%, 96%, 97%, 98%, or 99% complementary to the target region. In particular embodiments, the antisense oligonucleotide comprises least 8, 9, 10, 11, 12, 13, 14, 15, 16, 17, 18, 19 or 20 contiguous nucleobases that are 100% complementary to the target region.

The antisense oligonucleotide may comprise at least one modification, e.g. a nucleobase modification, a modification of the oligonucleotide backbone or a modification of a ribose sugar. In one embodiment, the antisense oligonucleotide comprises a modified sugar selected from among a 2′-O-methyl (2OMe), 2′-O-methoxy-ethyl (MOE), locked nucleic acids (LNA), 2′-fluoro or S-constrained-ethyl (cEt). In a further embodiment, the antisense oligonucleotide comprises backbone that comprises phosphorothioates.

Also provided are compositions comprising an antisense oligonucleotide of the present disclosure, such as pharmaceutical compositions.

In a further aspect, provided is a use of an antisense oligonucleotide for the treatment of a disorder associated with a heterozygous loss-of-function mutation in SCN2A, wherein the antisense oligonucleotide enhances splicing at a splice site of a retained intron in an intron-retaining SCN2A mRNA or pre-mRNA, wherein the retained intron is selected from among introns 1, 2, 3, 4, 5, 11, 13, 17 and 24 and wherein the antisense oligonucleotide comprises a sequence of nucleobases that is complementary to a target region in the SCN2A mRNA or pre-mRNA.

In yet another aspect, provided is a use of an antisense oligonucleotide for the treatment of a disorder associated with a heterozygous loss-of-function mutation in SCN2A, wherein the antisense oligonucleotide enhances splicing at a splice site of a retained intron in an intron-retaining SCN2A mRNA or pre-mRNA, wherein the retained intron is selected from among introns 2 and 17, wherein the antisense oligonucleotide comprises a sequence of nucleobases that is complementary to a target region in the SCN2A mRNA or pre-mRNA.

### BRIEF DESCRIPTION OF THE DRAWINGS

FIG. 1 is a schematic representation of intron retention in SCN2A, as analysed from information obtained from IRBase. The top panel shows a genomic map of SCN2A gene from UCSC browser. Thin lines represent the introns and the thick lines/blocks correspond to the exons. The bottom panel shows intron-retention events corresponding to introns in the genomic map. The height of the bars is indicative of the number of recorded events.

FIG. 2 is a schematic showing the design of primers for each exon-intron pair across the SCN2A sequence. A. Primers that are specific for intron-retaining transcripts: The forward primer was designed from the sequence of the preceding exon and the reverse primer from the sequence of the intron downstream to the exon. B. Primers specific to spliced transcripts: One of the primers was designed such that it spanned the junction of two nearby exons, while the other was designed from the sequence of the preceding or the succeeding exon accordingly.

FIG. 3 shows relative expression of introns in whole brain SCN2A mRNA obtained from two commercial sources. The expression of individual introns across the entire transcript was compared with the averaged exon expression. The results are a representation of three experiments, with the standard error of the mean indicated. A. mRNA from source 1—Ambion. B. mRNA from source 2—Takara.

FIG. 4 shows relative expression of introns in SCN2A mRNA from cell lines. The expression of individual introns across the entire transcript was compared with the averaged exon expression. The results are a representation of three experiments, with the standard error of the mean indicated. A. mRNA from SH-SY5Y cells. B. mRNA from SK-N-AS cells.

FIG. 5 is a schematic of the secondary structure prediction of SCN2A intron 2.

FIG. 6 is a graphical and schematic representation of modulation of SCN2A transcript expression in the presence of antisense oligonucleotides. Following transfection and incubation, the expression of SCN2A was analysed by qPCR. Mock transfected cells were used as a negative control. The housekeeping gene HPRT1 was used for normalization. The number of biological replicates ranges from 3 to 6. The regions highlighted in red and green are the predicted binding sites for HnRNPA1 and HnRNPI, respectively. A. Effect of antisense oligonucleotides targeting the 5′ end of SCN2A intron 2 on its expression. B. Effect of antisense oligonucleotides targeting the 3′ end of SCN2A intron 2 on its expression.

FIG. 7 shows the effect of antisense oligonucleotides INT2+6 (SEQ ID NO:126; targeting a region 6 base pairs downstream of 5′ of intron 2) on the expression of the SCN2A in is shown, using two sets of primers that cross the exon to exon boundaries of Exons 2 and 3 (A), and Exons 18 and 19 (B). In comparison, antisense oligonucleotide INT2+35 (SEQ ID NO:142; targeting a region 35 base pairs downstream of 5′ of intron 2) does not result in increase in SCN2A transcripts. Mock transfected cells were used as a negative control.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e. at least one or more than one) unless the context clearly dictates otherwise. Thus, for example, reference to “a polypeptide” includes a single polypeptide, as well as two or more polypeptides.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

An “antisense oligonucleotide” refers to a single-stranded oligonucleotide having a sequence that permits hybridization to a corresponding region or segment of a target nucleic acid. Reference to an antisense oligonucleotide includes reference to both unmodified and modified antisense oligonucleotides, wherein a modified antisense oligonucleotide contains at least one modified nucleoside and/or modified internucleoside linkage.

“Complementary,” as used herein, refers to the capacity for precise pairing between two nucleobases, such as between a nucleobase in an antisense oligonucleotide and a nucleobase in a SCN2A mRNA or pre-mRNA. The antisense oligonucleotide and the mRNA or pre-mRNA are complementary to each other when a sufficient number of corresponding positions in each molecule are occupied by nucleobases which can hydrogen bond with each other. Thus, “complementary” is used to indicate a sufficient degree of precise pairing over a sufficient number of nucleotides such that stable and specific binding occurs between the antisense oligonucleotide and the mRNA or pre-mRNA. It is understood that the antisense oligonucleotide need not be 100% complementary to the target region in the SCN2A mRNA or pre-mRNA to hybridize thereto. Moreover, an oligonucleotide may be complementary to, and hybridize, over one or more segments such that intervening or adjacent segments are not involved in the hybridization event. “Complementary” as used herein therefore includes reference to less than 100% complementary, such at least or about 70%, 75%, 80%, 85%, 90% or 95% sequence complementarity.

As used herein, a “disorder associated with a loss-of-function mutation in SCN2A” refers to a disorder that is associated with, is partially or completely caused by, or has one or more symptoms that are partially or completely caused by, a mutation in SCN2A that results in a loss-of-function phenotype, i.e. an decrease in the level (or amount) or activity of SCN2A.

As used herein, “expression of SCN2A” refers to the transcription of mRNA from SCN2A or the translation of protein from the SCN2A mRNA. SCN2A expression can be assessed using any method known in the art, including, but not limited to, Northern blot, Western blot and qRT-PCR.

As used herein, a “loss-of-function mutation” is a mutation in SCN2A that results in a decrease in expression and/or activity of the encoded SCN2A protein. Expression of the encoded SCN2A protein can be assessed using standard assays, such as Western blot. Typically, a loss-of-function mutation results in a decrease of at least or about 20%, 30%, 40%, 50%, 60%, 70%, 80%, 90%, 95% or more of the expression and/or activity of the encoded SCN2A protein. In some examples, the loss-of-function mutation results in a complete (i.e. 100%) loss of expression or activity of the encoded SCN2A protein, such as when the mutation is a mutation (e.g. nonsense mutation, large deletion or frameshift mutation) that results in the formation of a truncated transcript that is either not translated or is translated to a non-functional protein. A “heterozygous loss-of-function mutation” in SCN2A is one that is present in only one copy of SCN2A in the cell (i.e. one allele is a wild-type allele) and can lead to haploinsufficiency.

A “gapmer” as referred to herein is a chimeric antisense oligonucleotide in which an internal region having a plurality of nucleotides that support RNase H cleavage is positioned between external regions having one or more nucleotides, wherein the nucleotides comprising the internal region are chemically distinct from the nucleoside or nucleotides comprising the external regions.

As used herein, “hybridization” or “binding” or grammatical variations thereof means the pairing of substantially complementary strands of nucleic acids, such as between an antisense oligonucleotide of the disclosure and a SCN2A mRNA or pre-mRNA. One mechanism of pairing involves hydrogen bonding, which may be Watson-Crick, Hoogsteen or reversed Hoogsteen hydrogen bonding, between complementary nucleobases of the strands of nucleic acids. For example, adenine and thymine or uracil are complementary nucleotides which pair through the formation of hydrogen bonds. Hybridization can occur under varying circumstances. Reference to “hybridizes” or “binds” as used herein means that the antisense oligonucleotide hybridizes or binds to a target region in SCN2A mRNA or pre-mRNA by virtue of the complementarity in sequence between the antisense oligonucleotide and the target region, and does not significantly bind to a non-target region.

The terms “linked” and “attached” are used interchangeably and relate to any type of interaction that join two entities, such as an antisense oligonucleotide and a moiety (e.g. a cell penetrating peptide), and include covalent bonds or non-covalent bonds, such as, for example, hydrophobic/hydrophilic interactions, van der Waals forces, ionic bonds or hydrogen bonds.

The term “exon” refers to a portion of a gene that is present in the mature form of mRNA. Exons include the ORF (open reading frame), i.e., the sequence which encodes protein, as well as the 5′ and 3′ UTRs (untranslated regions). The UTRs are important for translation of the protein. Algorithms and computer programs are available for predicting exons in DNA sequences (e.g. Grail, Grail 2 and Genscan and US 20040219522 for determining an exon-intron junctions).

The term “intron” refers to a portion of a gene that is not translated into a wild-type protein and while present in genomic DNA and pre-mRNA, it is generally removed in the formation of mature mRNA by splicing.

The term “messenger RNA” or “mRNA” refers to RNA that is transcribed from genomic DNA and that carries the coding sequence for protein synthesis. The terms “precursor mRNA” or “pre-mRNA” refer to an immature single strand of messenger ribonucleic acid (mRNA) that contains one or more introns and that is directly transcribed from the DNA; for the purposes of the present disclosure, it is considered pre-mRNA until the poly(A) is added and 5′ and 3′ modifications take place. Pre-mRNA is transcribed by an RNA polymerase from a DNA template in the cell nucleus and is comprised of alternating sequences of introns and exons. In eukaryotes, pre-mRNA is processed into mRNA, which includes removal of the introns, i.e., “splicing”, and modifications to the 5′ and 3′ end (e.g., polyadenylation). mRNA typically comprises from 5′ to 3′; a 5′ cap (modified guanine nucleotide), 5′ UTR (untranslated region), the coding sequence (beginning with a start codon and ending with a stop codon), the 3′ UTR, and the poly(A) tail. Eukaryotic pre-mRNAs exist only transiently before being processed into mRNA. As described herein, polyadenylated transcripts in the nucleus of a cell can have one or more retained introns even after initial splicing of the primary transcript and addition of the poly(A) tail. For the purposes of the present disclosure, these transcripts are considered mRNA with retained introns. When a pre-mRNA has been properly processed to an mRNA, it is exported out of the nucleus and translated into a protein by ribosomes in the cytoplasm. The term “fully-spliced mRNA” as used herein means that the mRNA does not contain any introns, or does not contain the intron being targeted by the antisense oligonucleotides and methods according to the present disclosure.

As used herein, “nucleobase” means a heterocyclic moiety capable of pairing with a base of another nucleic acid, and includes, for example, adenine (A), guanine (G), cytosine (C), thymine (T) and uracil (U). Reference herein to nucleobase also includes reference to a modified nucleobase.

A “nucleoside” as used herein refers to a nucleobase linked to a sugar. Reference herein to a nucleoside also includes reference to a modified nucleoside, which has a modified sugar moiety or modified nucleobase. A “nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics e.g. non furanose sugar units.

As used herein, “nucleotide” refers to a nucleoside having a phosphate group covalently linked to the sugar portion of the nucleoside. Reference herein to a nucleotide also includes reference to a modified nucleotide, which has a modified sugar moiety, modified internucleoside linkage, or modified nucleobase. A “nucleotide mimetic” includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage).

The term “splicing” refers to the modification of a pre-mRNA following transcription, in which introns are removed and exons are joined. Pre-mRNA splicing involves two sequential biochemical reactions. Both reactions involve the spliceosomal transesterification between RNA nucleotides. In a first reaction, the 2′-OH of a specific branch-point nucleotide within an intron, which is defined during spliceosome assembly, performs a nucleophilic attack on the first nucleotide of the intron at the 5′ splice site forming a lariat intermediate. In a second reaction, the 3-OH of the released 5′ exon performs a nucleophilic attack at the last nucleotide of the intron at the 3′ splice site thus joining the exons and releasing the intron lariat.

As used herein, the term “sequence identity” or “% identical” or grammatical variations means that in a comparison of two sequences over a specified region the two sequences have the specified number or percentage of identical residues in the same position. Sequences can be aligned by any method known to those of skill in the art. Such methods typically maximize matches, and include methods such as using manual alignments and by using the numerous alignment programs available.

The term “splice site” refers to the junction between an exon and an intron in a pre-mRNA molecule (also known as a “splice junction”). The “splice site sequence” is the sequence surround the splice site that is capable of being recognised by the splicing machinery of the cell. A 5′ splice site (also referred to as a splice donor site) is the splice site at the 5′ end of the intron that marks the start of the intron and its boundary with the preceding exon sequence. A 3′ splice site (also referred to as a splice acceptor site) is the splice site at the 3′ end of the intron that marks the end of the intron and its boundary with the following exon sequence. Numbering used herein in reference to a 5′ splice site of an intron is therefore also in reference to the first nucleotide of the intron. Thus, for example, reference to position +1 relative to the 5′ splice site of an intron is reference to the first nucleotide in the intron sequence, e.g. reference to position +1 relative to the 5′ splice site of intron 2 is reference to nucleotide position 1 of the intron 2 sequence, e.g. position 1 of SEQ ID NO:12. In another example, reference to positions+18-+27 relative to the 5′ splice site of an intron is reference to the 18th through to the 27th nucleotide position of the intron sequence, e.g. position 18 through to position 27 of the intron 2 set forth in SEQ ID NO12. Reference to position −1 relative to the 5′ splice site of an intron is reference to the first nucleotide upstream to the intron sequence, e.g. reference to position −1 relative to the 5′ splice site of intron 2 is reference to nucleotide position 1 upstream of the intron 2 sequence, that is the last nucleotide of the adjacent exon. In another example, reference to positions −1 relative to the 3′ splice site of the intron 2 sequence, is reference to the final nucleotide of intron 2, e.g. final nucleotide of SEQ ID NO:12. In another example, reference to positions −10-−1 relative to the 3′ splice site of an intron is reference to the 10^{th} through to the 1^{st} nucleotide position upstream of the 3′ splice site, or the final nucleotide ten nucleotides of the intron.

As used herein the terms “treating” or “treatment” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and the like are to be considered in their broadest context. For example, treatment does not necessarily imply that a patient is treated until total recovery. In conditions which display or a characterized by multiple symptoms, the treatment or prevention need not necessarily remedy, prevent, hinder, retard, or reverse all of said symptoms, but may prevent, hinder, retard, or reverse one or more of said symptoms. In the context of the present disclosure, symptoms that may be ameliorated, reversed, prevented, retarded or the linked include but are not limited to seizures and spasms.

The term “subject” as used herein refers to an animal, in particular a mammal and more particularly a primate including a lower primate and even more particularly, a human who can benefit from the protocol of the present disclosure. A subject regardless of whether a human or non-human animal or embryo may be referred to as an individual, subject, animal, patient, host or recipient.

### Antisense Oligonucleotides for SCN2A

As demonstrated herein, introns 1, 2, 3, 4, 5, 11, 13, 17 and 24 are retained in mature SCN2A mRNA in brain tissue. Introns 2 and 17 in particular have relatively high retention rates. As demonstrated herein, a retained intron in SCN2A mRNA or pre-RNA can be targeted with antisense oligonucleotides so as to enhance splicing at the splice site of the retained intron, resulting in an increase in the amount of fully-spliced SCN2A mRNA (i.e. SCN2A mRNA that does not contain any intron). Such antisense oligonucleotides are therefore useful for increasing the amount of SCN2A produced by a cell, and thus useful as therapeutic agents for the treatment of disorders associated with heterozygous loss-of-function mutations in SCN2A, such as genetic epilepsy (including developmental and epileptic encephalocopathies (DEE)), intellectual disability, autism spectrum disorders and schizophrenia, where increasing the levels of SCN2A protein can provide a therapeutic effect.

Thus, provided herein are antisense oligonucleotides that enhance splicing at a splice site of a retained intron in an intron-retaining SCN2A mRNA or pre-mRNA, such as intron 1, 2, 3, 4, 5, 11, 13, 17 and 24. In particular examples, the antisense oligonucleotides enhances splicing at a splice site of intron 2 or intron 17 in an intron-retaining SCN2A mRNA or pre-mRNA.

The antisense oligonucleotide can function to enhance splicing in one of many ways. In one example, the antisense oligonucleotide binds to, or adjacent to, an intronic splicing silencer (ISS) (also referred to as an ISS site or ISS motif). ISS are cis-acting elements (i.e. sequences) in the RNA that play a role in silencing or inhibiting splicing at a splice site. The ISS is bound by a RNA-binding protein (RBP) that acts as a silencing repressor. Exemplary RBPs that act as repressors include heterogeneous nuclear ribonucleoproteins (hnRNPs), such as hnRNP A1, A2/B1, C1/C2, E1/E2/E3/E4, F, G, H, I, K, L, M, P, Q1/Q2/Q3 and U and hnRNP A2. The motifs recognised and bound by hnRNPs are not necessarily strict consensus sequences in the classical sense, but can be repeat elements (such as in the case of hnRNP L1, which recognises a CA repeat-rich element) or short and degenerate sequences. Moreover, hnRNPs may recognize specific structures rather than linear sequence motifs, such as in the case of hnRNP F (for review, see e.g. Geunes et al., 2016, Hum Genet. 135:851-867; Dvinge, 2018, FEBS Letters, 592:2987-3006). Notwithstanding this, algorithms are available to predict hnRNP binding motifs in RNA molecules (see e.g. Piva et al., 2009, Bioinformatics; Piva et al., 2012, Hum Mutat. 2012 January; 33(1):81-85). Binding of an antisense oligonucleotide to, or adjacent to, an ISS can prevent or inhibit binding of the RBP suppressor (e.g. an hnRNP, such as hnRNP A1), thereby enhancing splicing at a splice site of a retained intron, such as intron 1, 2, 3, 4, 5, 11, 13, 17, or 24. In other examples, the antisense oligonucleotide binds to a site in SCN2A mRNA or pre-mRNA that has a propensity to form an RNA secondary structure (e.g. stem, hairpin loop, pseuoknot, bulge, internal loop or multiloop), thereby reducing formation of the structure and facilitating efficient recruitment of splicing factors so as to enhance splicing of the retained-intron. In further examples, the antisense oligonucleotide binds to a sequence involved in the formation of G-quadruplexes, which can stabilise the G-quadruplex and enhance splicing (see e.g. Rouleau et al., 2015, Nucleic Acids Res. 43(1): 595-606; Ribeiro et al. 2015, Hum Genet. 134(1): 37-44). Those skilled in the art will appreciate that the composition and distribution of putative Quadruplex forming G-Rich Sequences (QGRS) of SCN2A can be predicted using the computational tool QGRS Mapper, wherein typically sequences with G scores above 19 are considered to have higher propensity for stable G4 structures.

In some examples, the antisense oligonucleotide of the present disclosure binds to nucleotides (or a target region) within the targeted intron, i.e. the intron for which enhanced splicing is to be effected, e.g. intron 1, 2, 3, 4, 5, 11, 13, 17 and 24. In other examples, the antisense oligonucleotide of the present disclosure binds to nucleotides (or a target region) in an adjacent exon, while still enhancing splicing at a splice site of the targeted intron.

In one example, the antisense oligonucleotide binds to a target region within intron 2 in the intron-retaining SCN2A mRNA or pre-mRNA. Accordingly, in some examples, an antisense oligonucleotide of the present disclosure has a sequence of nucleobases that is complementary to a sequence of nucleotides within intron 2 of SCN2A pre-mRNA, such as within the intron 2 set forth as follows:

(SEQ ID NO: 12)
GTGAGTTCTTAGTCAAGTTGCCTTCACTGCCTATTTA
CTAATTGGTTCTGGGCTAGTCCCAGGGATGAT
GGTGAAGAAGGCTGGCCTCCTTCCCTCTGTCTAAAGTATCACTAAGATGC
TGGATGGGCCTGACCGTGTAATGGACCAATGATCCTAGAAGTCTTTTGGA
AGCACTCATTTGAACCTGCATTTGTGAGACAGGCAGAGAACTGGTGAGGC
ATCCTCCAGCGCGGGAATTAAGGAAGGACAAAAGCCTATTCACCTTCTTG
AATACAAATTATATGCTTAAACCAGTGTAAATTGACCCTGATTCCCTAAT
AATGTTGAGAAGCAAAAACTGTAAACTAGGAGTCTATTTAAATTTTATTT
TTTATATTTGCAGGAGTAGTATCTAAATTCCTCTTTATAGTCTCTAGCTC
TCCATAAGTCACTTTGATCTTCAGTGGGTTTAATTATTCCTTTATACCAT
ACTTTCTCCTTTCTATTGCTCTCCACAGAAGGAATAATAGCAGGTGACTT
GTAGGTGCCAAATAAGATTCTGAGCAAAGAACACACCTGGAAAACCTTGA
AGTTCTCATGAGAAAATTTTCTAACCAAAAAAAAAAATCAAAGCCTCAAT
TTTGTGCTTTATGTGAATTATAAATGCGGTTTTAAAATACTTACATTAAA
ACTTGATAAAGTTGCTAAGAATTCCTATGGCATTGATCACAAATTTTCTT
AATAATCCTCATGTCATTTATCAAATTTAGGAAAGTTTATAGTGCTCAGA
AAAAAAAAGCATCTATCTTCATGTCATATGATGGTAATTATTATGTTATA
CACTATTTTACAGGGCAATATTTATAAATAATGGTTTTACTTTTCTCTTA
AAATATTCTTAATATATATTCTAAGTTTTATTTTATGTGTTGTGTTTTCT
TTTTCAG

In some examples, the antisense oligonucleotide binds to (i.e. comprises a sequence that is complementary to) a target region in intron 2 in an intron-retaining SCN2A mRNA or pre-mRNA, wherein the target region spans positions −6-+12, −5-+13, −4-+14, −3-+15, −2-+16, −1-+19, 0-+18, +1-+19, +2-+20, +3-21, +4-22, +5-+23, +6-+24, +7-+25, +8-26, +9-+27, +10-+28, +11-+29, +12-+30, +13-+31, +14-+32, +15-+33, +16-+34, +17-+35, +18-+36, +19-+37, +20-+38, +21-+39, +22-+40, +23-+41, +24-+42, +25-+43, +26-+44, +27-+45, +28-+46, +29-+47, +30-+48, +31-+49, +32-+50, +33-+51, +34-+52, +35-+53, +36-+54, +37-+55, +38-+56, +39-+57, +40-+58, +41-+59, +42-+60, +43-+61, +44-+62, +45-+63, +46-+64, +47-+65, +48-+66, +49-+67, +50-+68, +51-+69, +52-+70, +53-+71, +54-+72, +55-+73, +56-+74, +57-+75, +58-+76, +59-+77, +60-+78, +61-+79, +62-+80, +63-+81, +64-+82, +65-+83, +66-+84, +67-+85, +68-+86, +69-+87, +70-+88, +71-+89, +72-+90, +73-+91, +74-+92, +75-+93, +76-+94, +77-+95, +78-+96, +79-+97, +80-+98, +81-+99, relative to the 5′ splice site of intron 2. In particular embodiments, the antisense oligonucleotide binds to, or adjacent to, an ISS in intron 2. As determined herein, putative ISS recognised by hnRNPA1 are at positions+8-+12 (CTTAG), +33-+36 (ATTTA) or +60-+65 (CAGGGA) relative to the 5′ splice site of intron 2 (bolded in SEQ ID NO:12, above). Thus, in some embodiments, the antisense oligonucleotide binds to, or adjacent to, nucleotides at positions+8-+12, +33-+36 or +60-+65 relative to the 5′ splice site of intron 2. For example, the antisense oligonucleotide may bind to one or more of the nucleotides at position +4, +5, +6, +7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, +38, +39, or +40 relative to the 5′ splice site of intron 2, or may bind to one or more of the nucleotides at position +50, +51, +52, +53, +54, +55, +56, +57, +58, +59, +60, +61, +62, +63, +64, +65, +66, +67, +68, +69, or +70 relative to the 5′ splice site of intron 2.

In one example, the antisense oligonucleotide binds to a target region that spans or is within positions −6-+12, −5-+13, −4-+14, −3-+15, −2-+16, −1-+19, 0-+18, +1-+19, +2-+20, +3-21, +4-22, +5-+23, +6-+24, +7-+25, +8-26, +9-+27, +10-+28, +11-+29, +12-+30, +13-+31, +14-+32, +15-+33, +16-+34, +17-+35, +18-+36, +19-+37, +20-+38, +21-+39, +22-+40, +23-+41, +24-+42, +25-+43, +26-+44, +27-+45, +28-+46, +29-+47, +30-+48, +31-+49, +32-+50, +33-+51, +34-+52, +35-+53, +36-+54, +37-+55, +38-+56, +39-+57, +40-+58, +41-+59, +42-+60, +43-+61, +44-+62, +45-+63, +46-+64, +47-+65, +48-+66, +49-+67, +50-+68, +51-+69, +52-+70, +53-+71, +54-+72, +55-+73, +56-+74, +57-+75, +58-+76, +59-+77, +60-+78, +61-+79, +62-+80, +63-+81, +64-+82, +65-+83, +66-+84, +67-+85, +68-+86, +69-+87, +70-+88, +71-+89, +72-+90, +73-+91, +74-+92, +75-+93, +76-+94, +77-+95, +78-+96, +79-+97, +80-+98, +81-+99, relative to the 5′ splice site of intron 2 (e.g. the intron 2 set forth in SEQ ID NO:12), i.e. the antisense oligonucleotide has a sequence that is complementary to at least one the aforementioned regions.

In another example, the antisense binds to a target region that spans or is within positions −19-−1, −20-−2, −21-−3, −22-−4, −23-−5, −24-−6, −25-−7, −26-−8, −27-−9, −28-−10, −29-−11, −30-−12, −31-−13, −32-−14, −33-−15, −38-−20, −39-−21, −48-−30, −49-−31, −51-−33, −52-−34, −−53-−35, −54-−36, −62-−44, −64-−46, −65-−47, −66-−48, −67-−49, −76-−58, −77-−59, −78-−60, −79-−61, −82-−64, −83-65, −84-−66, −85-−67, −86-−68, −87-−69, −90-−72, −91-−73, −92-−74, −95-−77, −97-−79, −98-−80, −99-−81, −101-−83, −102-−84, −103-−85, −104-−86, −105-−87, −106-−88, −107-−89, −108-−90, −109-−91, −110-−92, −111-−93, −112-−94, −113-−95, −114-−96, −115-−97, −116-−98, −117-−99, relative to the 3′ splice site of intron 2 (e.g. the intron 2 set forth in SEQ ID NO:12), i.e. the antisense oligonucleotide has a sequence that is complementary to at least one of the aforementioned regions.

In some examples, the antisense oligonucleotide binds to, and thus comprises a sequence that is complementary to, positions −4-+14, −3-+15, −2-+16, +6-+24, +7-+25, +8-26, +9-+27, +10-+28, +14-+32, +19-+37, +20-+38, +21-+39, +22-+40, +23-+41, +24-+42, +25-+43, +26-+44, +27-+45, +28-+46, +29-+47, +30-+48, +31-+49, +32-+50, +33-+51, +34-+52, +35-+53, +43-+61, +45-+63, +46-+64, +49-+67, +52-+70, +59-+77, +64-+82, +65-+83, +68-+86, +69-+87, +70-+88, +71-+89, +72-+90 and +73-+91, relative to the 5′ splice site of intron 2. In some embodiments, the antisense oligonucleotide comprises a sequence having at least or about 70%, 80%, or 90% sequence identity to a sequence set forth in any one of SEQ ID NOs: 115-142, or a sequence having at least 8, 9, 10, 11, 12, 13, 14 or 15 contiguous nucleotides from a sequence set forth in any one of SEQ ID NOs: 115-142. In a particular embodiment, the sequence has at least or about 70%, 80%, or 90% sequence identity to a sequence set forth in any one of SEQ ID NO: 126 or SEQ ID NO: 138, or a sequence having at least 8, 9, 10, 11, 12, 13, 14 or 15 contiguous nucleotides from a sequence set forth in SEQ ID NO: 126 or SEQ ID NO:138.

In a further example, the antisense oligonucleotide binds to, and thus comprises a sequence that is complementary to, positions −19-−1, −21-−3, −30-−12, −31-−13, −32-−14, −62-−44, −64-−46, −67-−49, −76-−58, −77-−59, −78-−60, −79-−61 −82-−64, −83-65, −84-−66, −85-−67, −86-−68, −87-−69, −95-−77, −97-−79, −100-−82, −101-−83, −102-−84, −103-−85, −107-−89, −109-−91, −111-−93, −113-−95, and −114-−96, relative to the 3′ splice site of intron 2. the antisense oligonucleotide comprises the sequence set forth in any one of SEQ ID NOs:143-205, or a sequence comprising at least 8, 9, 10, 11, 12, 13, 14 or 15 contiguous nucleotides from a sequence set forth in any one of SEQ ID NOs:143-205.

In one embodiment, the antisense oligonucleotide binds to nucleotides within intron 17 in the intron-retaining SCN2A mRNA or pre-mRNA. Accordingly, in some examples, an antisense oligonucleotide of the present disclosure has a sequence of nucleobases that is complementary to a sequence of nucleotides within intron 17 of SCN2A (nucleotides 120271-130741 of NCBI Reference Sequence: NG_008143.1).

In one embodiment, the antisense oligonucleotide binds to nucleotides within intron 1 in the intron-retaining SCN2A mRNA or pre-mRNA. Accordingly, in some examples, an antisense oligonucleotide of the present disclosure has a sequence of nucleobases that is complementary to a sequence of nucleotides within intron 1 of SCN2A (nucleotides 5240-61371 of NCBI Reference Sequence: NG_008143.1).

In another embodiment, the antisense oligonucleotide binds to nucleotides within intron 3 in the intron-retaining SCN2A mRNA or pre-mRNA. Accordingly, in some examples, an antisense oligonucleotide of the present disclosure has a sequence of nucleobases that is complementary to a sequence of nucleotides within intron 3 of SCN2A (nucleotides 62735-73446 of NCBI Reference Sequence: NG_008143.1).

In yet another embodiment, the antisense oligonucleotide binds to nucleotides within intron 4 in the intron-retaining SCN2A mRNA or pre-mRNA. Accordingly, in some examples, an antisense oligonucleotide of the present disclosure has a sequence of nucleobases that is complementary to a sequence of nucleotides within intron 4 of SCN2A (nucleotides 73537-74264 of NCBI Reference Sequence: NG_008143.1).

In an embodiment, the antisense oligonucleotide binds to nucleotides within intron 5 in the intron-retaining SCN2A mRNA or pre-mRNA. Accordingly, in some examples, an antisense oligonucleotide of the present disclosure has a sequence of nucleobases that is complementary to a sequence of nucleotides within intron 5 of SCN2A (nucleotides 74394-74950 of NCBI Reference Sequence: NG_008143.1).

In an embodiment, the antisense oligonucleotide binds to nucleotides within intron 11 in the intron-retaining SCN2A mRNA or pre-mRNA. Accordingly, in some examples, an antisense oligonucleotide of the present disclosure has a sequence of nucleobases that is complementary to a sequence of nucleotides within intron 11 of SCN2A (nucleotides 81358-88754 of NCBI Reference Sequence: NG_008143.1).

In an embodiment, the antisense oligonucleotide binds to nucleotides within intron 13 in the intron-retaining SCN2A mRNA or pre-mRNA. Accordingly, in some examples, an antisense oligonucleotide of the present disclosure has a sequence of nucleobases that is complementary to a sequence of nucleotides within intron 13 of SCN2A (nucleotides 92584-96928 of NCBI Reference Sequence: NG_008143.1).

In yet another embodiment, the antisense oligonucleotide binds to nucleotides within intron 24 in the intron-retaining SCN2A mRNA or pre-mRNA. Accordingly, in some examples, an antisense oligonucleotide of the present disclosure has a sequence of nucleobases that is complementary to a sequence of nucleotides within intron 24 of SCN2A (nucleotides 146329-146691 of NCBI Reference Sequence: NG_008143.1).

The antisense oligonucleotides of the present disclosure can enhance splicing such that the amount or level of the fully-spliced SCN2A mRNA or the amount or level of SCN2A protein in the cell or population of cells that is contacted with the antisense oligonucleotide is increased by at least or about 20%, 30%, 40%, 50%, 60%, 70%, 80%, 90%, 100%, 110%, 120%, 130%, 140%, 150%, 200%, 250%, 300%, 350%, 400%, 450%, 500%, 600%, 700% or more compared to the amount or level of a fully-spliced SCN2A mRNA or of a SCN2A protein in a cell or population of cells that has not been contacted with an antisense oligonucleotide of the present disclosure. Thus, in some instances, the amount or level of a fully-spliced SCN2A mRNA or of a SCN2A protein in the cell or population of cells following exposure to an antisense oligonucleotide of the present disclosure is 1.2×, 1.3×, 1.4×, 1.5×, 1.6×, 1.7×, 1.8×, 1.9×, 2×, 2.1×, 2.2×, 2.3×, 2.4×, 2.5×, 3×, 3.5×, 4×, 4.5×, 5×, 6×, 7× or more compared to the amount or level of a fully-spliced SCN2A mRNA or of a SCN2A protein in a cell or population of cells that has not been exposed to an antisense oligonucleotide of the present disclosure. In some examples, the fully-spliced SCN2A mRNA is that described as NCBI Reference Sequence: NM_001040142.2 (SEQ ID NO:1), and/or derived from nucleotide sequence of NCBI Reference Sequence: NG_008143.1, (SEQ ID NO:2) and/or the SCN2A protein is that described as NCBI Reference Sequence: NP_001035232.1 (SEQ ID NO:3). In other examples, the fully-spliced SCN2A mRNA is that described as NCBI Reference Sequence: NM_001040143.2 (SEQ ID NO:4), and/or the SCN2A protein is that described as NCBI Reference Sequence: NP_001035233.1 (SEQ ID NO:5). In other examples, the SCN2A mRNA is that described as NCBI Reference Sequence: NM_001371246.1 (SEQ ID NO:6), and/or the SCN2A protein is that described as NCBI Reference Sequence: NP_001358175.1 (SEQ ID NO:7). In other examples, the SCN2A mRNA is that described as NCBI Reference Sequence: NM_001371247.1 (SEQ ID NO:8), and/or the SCN2A protein is that described as NCBI Reference Sequence: NP_001358176.1 (SEQ ID NO:9). In yet another example, the SCN2A mRNA is that described as NCBI Reference Sequence: NM_021007.3 (SEQ ID NO:10), and/or the SCN2A protein is that described as NCBI Reference Sequence: NP_066287.2 (SEQ ID NO:11).

The antisense oligonucleotides of the present disclosure are typically 8 to 50, nucleobases in length, such as 8 to 50, 8 to 40, 8 to 35, 8 to 30, 8 to 25, 8 to 20, 8 to 15, 9 to 50, 9 to 40, 9 to 35, 9 to 30, 9 to 25, 9 to 20, 9 to 15, 10 to 50, 10 to 40, 10 to 35, 10 to 30, 10 to 25, 10 to 20, 10 to 15, 11 to 50, 11 to 40, 11 to 35, 11 to 30, 11 to 25, 11 to 20, 11 to 15, 12 to 50, 12 to 40, 12 to 35, 12 to 30, 12 to 25, 12 to 20, or 12 to 15 nucleobases in length. Thus, in particular examples, the antisense oligonucleotides are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

The antisense oligonucleotides may be 100% complementary across their entire length to a target region of an intron-retaining SCN2A mRNA or pre-mRNA or may be less than 100% complementary. Typically, the antisense oligonucleotides are at least 70%, 80%, 85%, 86%, 87%, 88%, 89%, 90%, 91%, 92%, 93%, 94%, 95%, 96%, 97%, 98%, or 99% complementary to a target region of an intron-retaining SCN2A mRNA or pre-mRNA, such as a region identified above in intron 5, 8, 9, 12, 13, or 14. The antisense oligonucleotides may contain, for example, at least 8, at least 9, at least 10, at least 11, at least 12, at least 13, at least 14, at least 15, at least 16, at least 17, at least 18, at least 19, or at least 20 contiguous nucleobases that are complementary to a target region in an intron-retaining SCN2A mRNA or pre-mRNA. In instances where the antisense oligonucleotides are not 100% complementary, the mismatched or non-complementary nucleobase(s) can be clustered or interspersed with complementary nucleobases and need not be contiguous to each other. The non-complementary nucleobase(s) may be located at the 5′ end and/or 3′ end of the antisense compound. Alternatively, the non-complementary nucleobase(s) can be at an internal position of the antisense oligonucleotide. When two or more non-complementary nucleobases are present, they can be either contiguous or non-contiguous.

In particular embodiments, antisense oligonucleotides of the present disclosure are up to 10, 12, 13, 14, 15, 16, 17, 18, 19, 20, 21, 22, 23, 24, 25, 26, 27, 28, 29 or 30 nucleobases in length and comprise no more than 6, 5, 4, 3, 2, or 1 non-complementary nucleobase(s) relative to a target region in an intron-retaining SCN2A mRNA or pre-mRNA.

The antisense oligonucleotides of the present disclosure can be produced using any method known in the art. Typically, the antisense oligonucleotides are produced using chemical synthesis methods. While the antisense oligonucleotides can be unmodified, more typically the antisense oligonucleotides of the present disclosure contain one or more modifications. These modifications can function to, for example, increase stability of the antisense oligonucleotide (e.g. increase resistance of the antisense oligonucleotide to degradation by nucleases), increase affinity of the antisense oligonucleotide to the target mRNA or pre-mRNA, increase steric hindrance by the antisense oligonucleotide, increase RNase H activity, and/or improve intracellular uptake. Exemplary modifications that are well known to those skilled in the art include, but are not limited to, modification of the nucleobase, modification of the backbone phosphate linkages (e.g. phosphodiester, phosphoramidate, or phosphorothioate (PS) modification), modifications of the ribose sugar (e.g. 2′-O-methyl (2OMe), 2′-O-methoxy-ethyl (MOE), locked nucleic acids (LNA), 2′-fluoro and S-constrained-ethyl (cEt) modifications) and other modifications such as replacement of the entire sugar phosphate backbone with polyamide linkages to produce peptide nucleic acids (PNA) and the use of a morpholine ring instead of the ribose ring and phosphoroamidate intersubunit linkages to produce phosphorodiamidate morpholino oligomers (PMO) (broadly reviewed in, for example, Sardone et al. (2017) Molecules 22(4): 563 Evers et al. (2015) Adv Drug Del Rev 87:90-103; Kole et al. (2012) Nat Rev Drug Discov. 11(2): 125-140).

In particular embodiments, the antisense oligonucleotides of the present disclosure contain one or more modified nucleobases. These can function to, for example, increase stability or binding affinity of the antisense oligonucleotide. Exemplary modified nucleobases include, but are not limited to, N⁶-methyladenine, N²-methylguanine, hypoxanthine, 7-methylguanine, 5-methylcytosine, 5-hydroxymethylcytosine, pseudouracil, 4-thiouracil, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N⁴-ethylcytosine, or derivatives thereof; N²-cyclopentylguanine (cPent-G), N2-cyclopentyl-2-aminopurine (cPent-AP), and N²-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). In particular embodiments, the antisense oligonucleotides contain one or more modified nucleobases that increase the binding affinity of the antisense oligonucleotide to the SCN2A mRNA or pre-mRNA, such as 5-methylcytosine (5-me-C), 5-substituted pyrimidines, 6-azapyrimidines and N-2, N-6 and 0-6 substituted purines, including 2 aminopropyladenine, 5-propynyluracil and 5-propynylcytosine.

The antisense oligonucleotides of the present disclosure may comprise modified sugar moieties. Exemplary sugar moiety modifications include 2′-O-methyl (2OMe), 2′-O-methoxy-ethyl (MOE), locked nucleic acids (LNA), 2′-fluoro and S-constrained-ethyl (cEt) modifications.

In particular embodiments, the backbones of the antisense oligonucleotides of the present disclosure comprise phosphorothioates, chiral phosphorothioates, phosphorodithioates, phosphotriesters, aminoalkylphosphotriesters, methyl or other alkyl phosphonates comprising 3′alkylene phosphonates or chiral phosphonates, phosphinates, phosphoramidates comprising 3′-amino phosphoramidate and aminoalkylphosphoramidates, thionophosphoramidates, thionoalkylphosphonates, thionoalkylphosphotriesters, or boranophosphates. In other embodiments, the backbone has no phosphorus atom. Exemplary oligonucleotide backbones that do not include a phosphorus atom include those that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside; see e.g. U.S. Pat. Nos. 5,698,685, 5,217,866, 5,142,047, 5,034,506, 5,166,315, 5,185,444, 5,521,063, 5,506,337, 8,076,476, 8,299,206 and 7,943,762); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In one example, the antisense oligonucleotides of the present disclosure are a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone (see e.g. U.S. Pat. Nos. 5,539,082; 5,714,331; and 5,719,262).

In particular embodiments, the antisense oligonucleotides of the present disclosure are partially or completely resistant to RNase H. Such antisense oligonucleotides can include 2′-O-methyl derivatives, and/or phosphorothioate backbones, both of which are resistant to nuclease degradation. In further examples, the antisense oligonucleotides do not activate RNase H, typically by virtue of the presence of one or more structural modifications that sterically hinders or prevent binding of RNase H to a duplex molecule containing the antisense oligonucleotide and the SCN2A mRNA or pre-mRNA. For example, such antisense oligonucleotides include those where at least one, or all, of the inter-nucleotide bridging phosphate residues are modified phosphates, such as methyl phosphonates, methyl phosphorothioates, phosphoromorpholidates, phosphoropiperazidates and phosphoramidates. For example, every other one of the internucleotide bridging phosphate residues may be modified as described. In another non-limiting example, such antisense molecules are molecules wherein at least one, or all, of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl).

In other examples, the antisense oligonucleotides of the present disclosure activate Rnase H when they form a DNA-RNA duplex with the SCN2A mRNA or pre-mRNA. Exemplary of such antisense oligonucleotides are gapmers, which are chimeric molecules containing at least one region modified so as to confer increased resistance to nuclease degradation, increased cellular uptake, increased binding affinity for the target nucleic acid, and a second region that serves as a substrate for Rnase H. Gapmers have an internal region having a plurality of nucleotides that support Rnase H cleavage. This internal region is positioned between external regions having a plurality of nucleotides that are chemically distinct from the nucleosides of the internal region, and which serve to, for example, increase stability of the antisense oligonucleotide and protect it from nuclease degradation. In certain embodiments, the external regions of the gapmer contain β-D-ribonucleosides, β-D-deoxyribonucleosides, 2′-modified nucleosides (e.g. 2′-MOE, and 2′-O—CH₃, among others), bridged nucleic acids (BNAs), or locked nucleic acids (LNAs).

The antisense oligonucleotides of the present disclosure may also be linked to one or more one or more moieties that enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or 25damantine acetic acid, a palmityl moiety, or an octadecylamine or hexylamino-carbonyl-oxycholesterol moiety, carbohydrates, phospholipids, biotin, phenazine, folate, phenanthridine, anthraquinone, acridine, fluoresceins, rhodamines, coumarins, and various dyes.

In particular embodiments, the antisense oligonucleotides are linked to a cell-penetrating peptide (CPP) that is effective to enhance transport of the compound into cells. The transport moiety can be attached to either terminus of the antisense oligonucleotide, resulting in increased penetration of the antisense oligonucleotides into cells and macromolecular translocation within multiple tissues in vivo upon systemic administration. In one embodiment, the cell-penetrating peptide is an arginine-rich peptide transporter. Antisense oligonucleotides linked with arginine-rich CPPs were able to cross the blood-brain barrier and were widely distributed throughout the brain of wild-type mice following systemic delivery (Du et al. Hum. Mol. Genet., 20 (2011), pp. 3151-3160). In another embodiment, the cell-penetrating peptide may be Penetratin or the Tat peptide. These peptides are well known in the art and are disclosed, for example, in US Publication No. 20100016215. The transport moieties described above have been shown to greatly enhance cell entry of attached oligomers, relative to uptake of the oligomer in the absence of the attached transport moiety. For example, antisense oligonucleotides linked with arginine-rich CPPs were able to cross the blood-brain barrier and were widely distributed throughout the brain of wild-type mice following systemic delivery (Du et al. Hum. Mol. Genet., 20 (2011), pp. 3151-3160). Uptake may be enhanced at least ten-fold, or at least twenty-fold, relative to the unconjugated compound. In other examples, the antisense oligonucleotide is coupled to a dopamine reuptake inhibitor (DRI), a selective serotonin reuptake inhibitor (SSRI), a noradrenaline reuptake inhibitor (NRI), a norepinephrine-dopamine reuptake inhibitor (NDRI), or a serotonin-norepinephrine-dopamine reuptake inhibitor (SNDRI), as described in, e.g., U.S. Pat. No. 9,193,969. In further examples, the antisense oligonucleotides are conjugated to peptides collectively known as “angiopeps” which are capable of crossing the blood-brain barrier by receptor-mediated transcytosis using the low-density lipoprotein receptor-related protein-1 (LRP-1), and which allow the delivery of systemically administered antisense-peptide conjugates to the brain (see e.g. WO200979790).

The antisense oligonucleotides can also be modified to have one or more stabilizing groups that are generally attached to one or both termini to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps.

### Assessment of the Antisense Oligonucleotides

Antisense oligonucleotides of the present disclosure can be designed rationally, so as to target a specific region or site in an intron (e.g. an ISS, a G-quadruplex or a region with a propensity for secondary structure) and/or by methods such as antisense microwalk or tiling that cover the whole intron or just a region of an intron. For example, the antisense oligonucleotides used in the antisense walk can be tiled every 1, 2, 3, 4, 5, 6, 7, 8, 9 or 10 nucleotides from approximately 100 nucleotides upstream of the 5′ splice site of the retained intron (e.g. in the preceding exon) to approximately 100 nucleotides downstream of the 5′ splice site and/or from approximately 100 nucleotides upstream of the 3′ splice site of the retained intron to approximately 100 nucleotides downstream of the 3′ splice site of the target/retained intron (e.g. in the following exon). The activity of these antisense oligonucleotides can then be assessed and confirmed using various techniques known in the art. For example, the ability of the antisense oligonucleotides to enhance splicing, and thereby increase production of fully-spliced SCN2A mRNA and/or SCN2A protein can be assessed using in vitro assays to confirm that the antisense oligonucleotides are suitable for use in the methods of the present disclosure. Mouse models can be used to not only assess the ability of the antisense oligonucleotides to increase the level or amount of fully-spliced SCN2A mRNA and/or SCN2A protein in vivo, but to also ameliorate symptoms associated with heterozygous loss-of-function SCN2A mutations.

In one example, cells such as mammalian neuronal cells (e.g. SH-SY5Y or SK-N-AS cells) are transfected with an antisense oligonucleotide of the present disclosure. The levels of fully-spliced SCN2A mRNA and intron-retaining SCN2A mRNA can be assessed using qRT-PCR or Northern blot as is well known in the art. The level SCN2A protein can also be assessed, such as by Western blot on total cell lysates or fractions.

The levels of fully-spliced SCN2A mRNA, intron-retaining SCN2A mRNA and/or SCN2A protein observed when cells are exposed to an antisense oligonucleotide of the present disclosure are compared to the respective levels observed when cells are exposed with a negative control antisense oligonucleotide, so as to determine the change resulting from the antisense oligonucleotide of the present disclosure. Typically, the level of fully-spliced SCN2A mRNA and/or SCN2A protein is increased by at least or about 20%, 25%, 30%, 35%, 40%, 45%, 50%, 55%, 60%, 65%, 70%, 75%, 80%, 85%, 90%, 100%, 110%, 120%, 125%, 30%, 135%, 140%, 145%, 150%, 155%, 160%, 165%, 170%, 175%, 180%, 185%, 190%, 200%, 250%, 300%, 350%, 400% or more. In such instances, the antisense oligonucleotides of the present disclosure can be used for treating a disease or condition associated with a heterozygous loss-of-function mutation in SCN2A.

Mouse models can also be used to assess and confirm the activity of the antisense oligonucleotides of the present disclosure. For example, an antisense oligonucleotide can be administered to a heterozygous SCN2A knockout mouse, which displays physical and behavioural traits similar to those observed in patients with SCN2A-related intellectual disability (see e.g. Nakajima et al. 2019, Neuropsychopharmacol Rep. 39(3):223-237; Guo et al., 2009, Neuropsychopharmacol. 2009 34(7):1659-72). The ability of the antisense oligonucleotides of the present disclosure to enhance splicing, increase the levels of fully-spliced SCN2A mRNA and/or SCN2A protein, and/or ameliorate any symptoms associated with the SCN2A mutation can then be assessed. In a particular example, SCN2A mRNA and/or protein levels in the brain, and in particular the neurons, are assessed. The levels of fully-spliced SCN2A mRNA, intron-retaining SCN2A mRNA and/or SCN2A protein following administration of an antisense oligonucleotide of the present disclosure are compared to the respective levels observed when a negative control antisense oligonucleotide is administered to the mice, so as to determine the change resulting from the antisense oligonucleotide of the present disclosure. Typically, the level of fully-spliced SCN2A mRNA and/or SCN2A protein is increased by at least or about 20%, 25%, 30%, 35%, 40%, 45%, 50%, 55%, 60%, 65%, 70%, 75%, 80%, 85%, 90%, 100%, 110%, 120%, 125%, 30%, 135%, 140%, 145%, 150%, 155%, 160%, 165%, 170%, 175%, 180%, 185%, 190%, 200%, 250%, 300%, 350%, 400% or more. In another example, the effect of administration of an antisense oligonucleotide of the present disclosure on the physical and/or behavioural traits of the mice is assessed. For example, behavioural and electrophysiological measures of memory and seizure in the mice can be assessed as described by Creson et al. (eLife 2019; 8: e46752).

### Compositions

The present disclosure provides compositions comprising the antisense oligonucleotides described above and herein. In particular examples, provided are pharmaceutical compositions comprising the antisense oligonucleotides and a pharmaceutically acceptable carrier. The compositions can also comprise additional ingredients such as carriers, diluents, stabilizers and excipients. The compositions can include one or more than one antisense oligonucleotide (e.g. two or more antisense oligonucleotides targeting the same or different introns), and further may comprise one or more other therapeutic agents.

The carriers, diluents, stabilizers and excipients can include buffers such as phosphate, citrate, or other organic acids; antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

The antisense oligonucleotides may also be formulated in compositions with liposomes, nanoparticles, microparticles, microspheres, lipid particles, vesicles, and the like, for the introduction of the antisense oligonucleotides of the present disclosure into cells. In embodiments, a penetration enhancer is included to effect the efficient delivery of the antisense oligonucleotide, e.g., to aid diffusion across cell membranes and/or enhance the permeability of a lipophilic drug. In some embodiments, the penetration enhancer is a surfactant, fatty acid, bile salt, chelating agent, or non-chelating nonsurfactant.

In embodiments, the antisense oligonucleotide is formulated in the context of a viral vector (e.g. adeno-associated viral (AAV) vector) where the vector comprises a genome that encodes an antisense oligonucleotide of the present disclosure.

Compositions comprising the antisense oligonucleotides encompass compositions comprising any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure also provides pharmaceutically acceptable salts of the antisense oligonucleotides described herein and other bio equivalents. Suitable pharmaceutically acceptable salts include, but are not limited to, sodium and potassium salts.

### Methods

The antisense oligonucleotides described above and herein can be used to increase levels of fully-spliced SCN2A mRNA and/or SCN2A protein in a cell (e.g. a neuronal cell) or in a subject. Consequently, the antisense oligonucleotides described above and herein can be used to treat a disorder associated with a heterozygous loss-of-function mutation in SCN2A, e.g. epileptic encephalopathy or autism or intellectual disability associated with a heterozygous loss-of-function mutation in SCN2A. The methods of the present disclosure therefore include a step of contacting a cell to an antisense oligonucleotide of the present disclosure, and/or administering an antisense oligonucleotide of the present disclosure to a subject. As would be appreciated, the phrase “administering an antisense oligonucleotide” and grammatical variations thereof encompasses embodiments where a composition comprising the antisense oligonucleotide is administered to subject, and embodiments where a composition comprising an agent that encodes the antisense oligonucleotide (e.g. a viral vector) is administered to subject. In the latter embodiment, it is understood that the antisense oligonucleotide is expressed in vivo, thereby effecting administration of the antisense oligonucleotide to the subject.

In some examples, the subject presenting with a disease or condition that may be associated with a heterozygous loss-of-function mutation in SCN2A is genotyped to confirm the presence of a known heterozygous loss-of-function mutation in SCN2A prior to administration of the antisense oligonucleotides and compositions thereof. For example, whole exome sequencing can be performed on the subject. Known heterozygous loss-of-function mutations in SCN2A may include, but are not limited to, those described in Vlaskamp et al. (Neurology, 2019, 92(2): e96-e97). In other examples, the subject is first genotyped to identify the presence of a mutation in SCN2A and this mutation is then confirmed to be a loss-of-function mutation, e.g. by assessing the levels of SCN2A mRNA or protein.

The precise amount or dose of the antisense oligonucleotide administered to the subject depends on, for example, the efficacy of the antisense oligonucleotide, the presence of other moieties (e.g. CCPs), the route of administration, the number of dosages administered, and other considerations, such as the weight, age and general state of the subject. Particular dosages and administration protocols can be empirically determined or extrapolated from, for example, studies in animal models or previous studies in humans, or may be otherwise determined by those skilled in the art using standard procedures.

The antisense oligonucleotides can be administered by any method and route understood to be suitable by a skilled artisan. Typically, the antisense oligonucleotides are administered parenterally, such as by subcutaneous administration, intravenous administration, intramuscular administration, intraarterial administration, intraperitoneal administration, or intracranial administration, e.g., intrathecal or intracerebroventricular administration. In other embodiments, the antisense oligonucleotides are delivered intranasally. Administration of the antisense oligonucleotides in the methods described herein preferably results in delivery of the antisense oligonucleotides to the central nervous system. In particular embodiments, the antisense oligonucleotides are administered intrathecally or by intracerebroventricular administration. The methods of the present disclosure can involve any combination of any two or more routes.

The antisense oligonucleotides can be administered to a subject one time or more than one time, including 2, 3, 4, 5 or more times. Where the antisense oligonucleotides are administered more than one time, the time between dosage administration can be, for example, 1, 2, 3, 4, 5, 6, 7, 8, 9, 10, 11, 12 or more weeks, or 1, 2, 3, 4, 5, 6, 7, 8, 9, 10, 11, 12 or more months. Selecting an optimal protocol is well within the level of skill of the skilled artisan and may depend on, for example, the half-life of the antisense oligonucleotide and the severity of the condition. In a particular embodiment, the antisense oligonucleotides are administered about every 3 months.

The antisense oligonucleotides, if desired, can be presented in a package, in a kit or dispenser device, such as a syringe with a needle, or a vial and a syringe with a needle, which can contain one or more unit dosage forms. The kit or dispenser device can be accompanied by instructions for administration.

In order that the disclosure may be readily understood and put into practical effect, particular exemplary embodiments will now be described by way of the following non-limiting examples.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

### Example 1—Materials and Methods

### Cell Culture

The cell lines SH-SY5Y and SK-N-AS were obtained from ECACC (European Collection of Authenticated Cell Cultures). The human brain RNA was purchased from Ambion, USA and from Takara-Bio, USA. The cells were grown in Dulbeccos modified Eagle's medium (DMEM) supplemented with 10% FBS. The cells were maintained at 37° C. and 5% CO₂. Frozen stocks of the cells were made within passage 6 and stored in liquid nitrogen. Cells were used for experiments below a passage number of 20.

### Cell Transfection

Cells were plated in a 96 well plate at a density of 5,000-10,000 cells per well. Transfections were carried out following 18-24 hours of incubation. The instructions provided with the Lipofectamine 3000 transfection reagent from Thermofisher Scientific were followed. Briefly, the transfection reagent was mixed with OptiMEM in one tube, and the ASO was mixed with the same in another tube. The contents of both the tubes were gently mixed together, and following an incubation, the transfection complex was layered onto the cells containing fresh medium. The cells were then incubated for the required time.

### RNA Isolation

Total RNA was extracted from cells treated with ASO for the required time periods using the Qiagen Rneasy minikit. Briefly, the cells were pelleted, and RNA was isolated according to the manufacturer's instructions. The genomic DNA elution column was used to free the RNA of any contaminating genomic DNA. RNA quantity and integrity were determined by nanodrop.

### Reverse Transcription

A total of 500 ug RNA was reverse transcribed to cDNA using the Promega M-MLV reverse transcriptase enzyme as per the kit instructions. The first strand synthesis of cDNA was performed using OligodT primers, which ensured that only mature polyadenylated RNA transcripts were reverse transcribed.

### Intron Retention Analysis

cDNA from the various tissue/cell samples were analysed using the GoTaq® green kit provided by Promega. The cDNA was added to the reaction mastermix and pipetted into the qPCR plate. The primers for each exon-exon and exon-intron pair were then pipetted into the wells. Each reaction had a technical duplicate and a water control. Absence of genomic DNA contamination was ensured with the RT minus reactions.

### Antisense Oligonucleotide (ASO) Screening Using the Taqman® Fast Advanced Cells to Ct Kit

Cells were seeded in 96 well plates at the required density. Within the following 18-24 hours, transfections were carried out and the cells were incubated up to the time of screening. The further experimental procedures were carried out according to the manufacturer's instructions. Briefly, the medium was aspirated, and the cells were washed with cold PBS. Dnase-containing lysis solution was added to the cells and incubated for 5 min at room temperature. Stop solution was then added to the cells and the cells were incubated for 2 min at room temperature in order to halt the lysis. A total of 20% of lysate was used in the conversion to cDNA using the kit components for reverse transcription. The cDNA was then added to the master mix made using the components for qPCR provided in the kit at a concentration of 25%. Taqman® primers were used for the assay. Each reaction was duplexed with a housekeeping gene for in-well normalization of expression. The ASO treated cells were normalized to mock transfected cells.

### Relative Gene Expression Assay

Cell lysates were collected 24 hours after transfection, and used in taqman based gene expression assays. SCN2A expression levels were measured using the Cells to CT kit (Invitrogen) real-time RT-PCR kit. Taqman probes targeting the SCN2A exon 2-3 boundary were used, with housekeeping HPRT (Hypoxanthine Guanine Phosphoribosyltransferase) gene used as a control.

### Example 2—Intron Retention in Human SCN2A

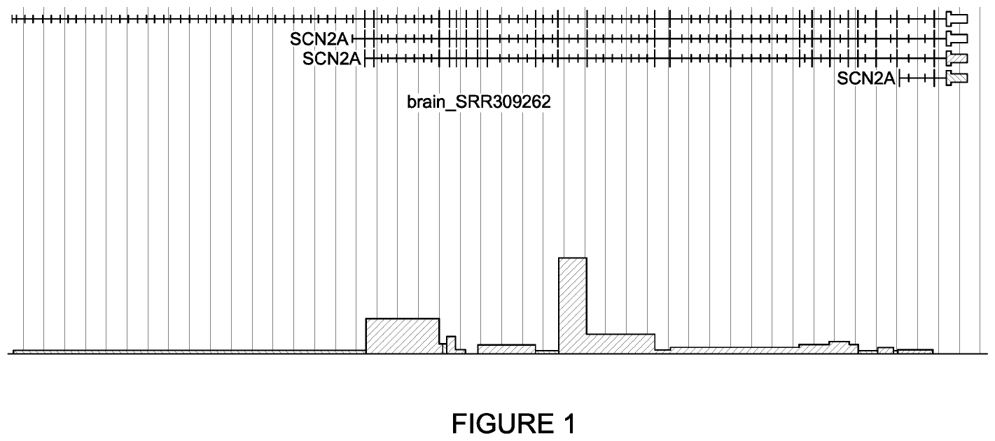

IRBase (Middleton et al., 2017, Genome Biol. 18: 51) is an RNA sequencing resource of over 2000 human samples, in which a specific intron retention event of a gene in a particular tissue can be assessed. Using this database, the events of intron retention of SCN2A in brain tissue was analysed. As shown in FIG. 1, several introns of SCN2A exhibit retention, with intron 11 showing the highest number of events (thin lines represent the introns and the thick lines/blocks correspond to the exons; the height of the bars is indicative of the number of recorded events of intron retention).

In vitro validation of the intron retention was carried out by analysing the levels of sequences corresponding to introns by quantitative PCR. To achieve this, primers were designed that would specifically detect the presence of retained introns relative to the exons flanking them. The use of cDNA that had been reverse transcribed from DNase-treated polyadenylated RNA ensured that pre-mRNA transcripts, and not genomic DNA, was detected by the primers.

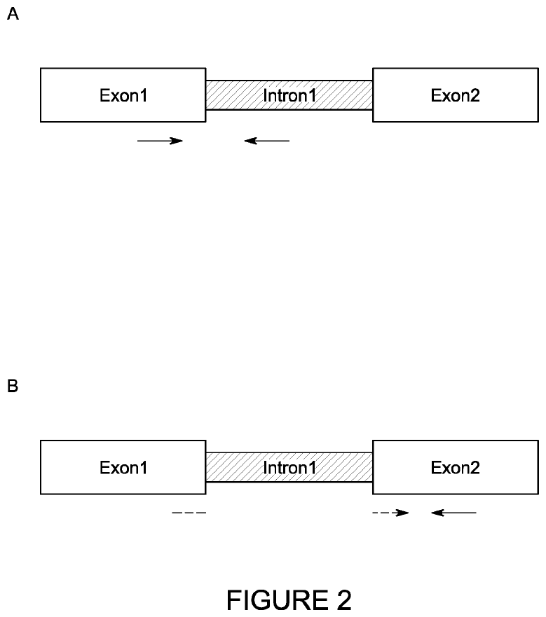

Intron retention events in the mature SCN2A mRNA were analysed by real-time PCR. Two sets of primers were designed for each Exon-Intron pair across the SCN2A sequence (N_008143.1; SEQ ID NO:2), consisting of 27 exons and 26 introns:
A: primers that are specific for intron-retaining transcripts: The forward primer was designed from the sequence of the preceding exon and the reverse primer from the sequence of the intron downstream to the exon (FIG. 2A).
B: primers specific to spliced transcripts: One of the primers was designed such that it spanned the junction of two nearby exons, while the other was designed from the sequence of the preceding or the succeeding exon accordingly (FIG. 2B).

The primers are set forth below in Table 1.

TABLE 1
Primers for intron retention analysis

| | | SEQ |
|---|---|---|
| | | ID |
| Primer name | Primer sequence | NO. |
| SCN2A E1-E2F | CTTTATTTCAGCACTTTCTTATGCAAG | 13 |
| SCN2A E1-E2R | TCTTCTGCAATGCGTTGTTC | 14 |
| SCN2A E1-I1F | TTTTTCCCTCCCTGTTTCTG | 15 |
| SCN2A E1-I1R | TCTTCTGCAATGCGTTGTTC | 16 |
| SCN2A E2-E3F | GACCCCTACTATATCAATAAGAAAACG | 17 |
| SCN2A E2-E3R | AGGGTTGAAGGGAGTTAAAATG | 18 |
| SCN2A E2-I2F | AGAAAGTGAGTTCTTAGTCAAGTTGC | 19 |
| SCN2A E2-I2R | TGCAGGTTCAAATGAGTGCT | 20 |
| SCN2A E3, 4, | GGTACATTCTTTATTCAATATGCTCATTA | 21 |
| 5F | | |
| SCN2A E3, 4, | TCTAAACAAAAGCCCCTTGC | 22 |
| 5R | | |
| SCN2A E3-I3F | GGTACATTCATATCCTTTTTCAAATC | 23 |
| SCN2A E3-I3R | CACTTAGAAGACGGGGGAAA | 24 |
| SCN2A I4-E4F | ATATCGTAGGGGGACCAACC | 25 |
| SCN2A I4-E4R | TCTAAACAAAAGCCCCTTGC | 26 |
| SCN2A E5-E6F | CACAGTCATTACTTTTGCATATGTG | 27 |
| SCN2A E5-E6R | TGTCTGAAATTGTTTTCAATGCT | 28 |
| SCN2A I5-E6F | AAGGTGGTACGCCCCTTATATC | 29 |
| SCN2A I5-E6R | TTGTTTTCAATGCTCGGAGA | 30 |
| SCN2A E6-E7F | ATTCCAGACCTGAAGACCATT | 31 |
| SCN2A E6-E7R | GAGGCCATTGGAAACATTTA | 32 |
| SCN2A E6-I6F | TTCTCCGAGCATTGAAAACA | 33 |
| SCN2A E6-I6R | GCAAAGAATCAAATCCATCCA | 34 |
| SCN2A E7-E8F | CCTGAAGACCATTGTGGGGG | 35 |
| SCN2A E7-E8R | AAAGTGACTTTTATCCTC | 36 |
| SCN2A E7-I7F | TGTTTGCAATGGCCTCCAGA | 37 |
| SCN2A E7-I7R | AGTCTAAGTCACTTGATTCACATCT | 38 |
| SCN2A E8-E9F | AGATGCACGCCAGTGTCCT | 39 |
| SCN2A E8-E9R | CAGTTGATAAAGGTTTTCCCAGA | 40 |
| SCN2A E8-I8F | TAGAGGGGCAAAATGATGCT | 41 |
| SCN2A E8-I8R | TGGAGGGTTTTGAGTGAGGA | 42 |
| SCN2A E9-E10F | CTTTATCAACTGACACTACGTGCT | 43 |
| SCN2A E9-E10R | CCTTCTGTTCAGCCTCTTCC | 44 |
| SCN2A E9-I9F | ACCCCAACTATGGCTACACG | 45 |
| SCN2A E9-I9R | GCAATTCTCTTGAGTTCGGTGT | 46 |
| SCN2A E10-E11F | CAAGAAGAAGCTCAGGCGG | 47 |
| SCN2A E10-E11R | CGATTTTCGGACTCTGTCATT | 48 |
| SCN2A E10-I10F | AGGTTTCCGTTTTTCCTTGG | 49 |
| SCN2A E10-I10R | AAGGCGGTTTTCTCTCCATT | 50 |
| SCN2A E11-E12F | CTCCACACCAGTCCTTACTGAGO | 51 |
| SCN2A E11-E12R | TTGTCCTCAAAGGTGCTGTG | 52 |
| SCN2A E11-I11F | AGGTTTCCGTTTTTCCTTGG | 53 |
| SCN2A E11-I11R | AAGGCGGTTTTCTCTCCATT | 54 |
| SCN2A E12-E13F | CTACCAGAGGGCACAACTACTG | 55 |
| SCN2A E12-E13R | CATTGCTCTTTGCCTTGATG | 56 |
| SCN2A E12-I12F | GACTGCAATGGTGTGGTCTC | 57 |
| SCN2A E12-I12R | TGGGCAACAAACTCAACAAA | 58 |
| SCN2A E13-E14F | CATGGAAGAACTTGAAGAATCCA | 59 |
| SCN2A E13-E14R | TGGGATAGTGCTCCATAGCC | 60 |
| SCN2A E13-I13F | TTTTGACCAACACCATGGAA | 61 |
| SCN2A E13-I13R | GCAGCTTTCTGGAGGCTAAA | 62 |
| SCN2A E14-E15F | TGGAAACCTGGTCTTCACAG | 63 |
| SCN2A E14-E15R | CGGAATGATCGGAGAACTG | 64 |
| SCN2A E14-I14F | CCATCACCATCTGCATTGTC | 65 |
| SCN2A E14-I14R | GGGATGCTAAACATTTGATGTC | 66 |
| SCN2A E15-E16F | GGCTGCTCCGAGTTTTCAA | 67 |
| SCN2A E15-E16R | ACACGATCAGGAAGGAGTGG | 68 |
| SCN2A E15-I15F | GGCAAATGTGGAAGGATTGT | 69 |
| SCN2A E15-I15R | TGGTGGTATGGCAATCGAAT | 70 |
| SCN2A E16-E17F | GGAAATCTAGTGCTTCTGAACCTC | 71 |
| SCN2A E16-E17R | GCTTTCTGCTTCCTAACAAAGG | 72 |
| SCN2A E16-I16F | GCCAAACCATGTGCCTTACT | 73 |
| SCN2A E16-I16R | GACAATAGGAAGTGGCCTTGA | 74 |
| SCN2A E17-E18F | GCAAAGAGAAGCTAAATGCAAC | 75 |
| SCN2A E17-E18R | GCCTTCAGGTTCAAGGGATT | 76 |
| SCN2A E17-I17F | CCAAATGCTGTTGGAGAATC | 77 |
| SCN2A E17-I17R | ACAGGAAGGAAACACGCAAT | 78 |
| SCN2A E18-E19F | CTGTTTTACAGAAGACTGTGTACGG | 79 |
| SCN2A E18-E19R | GCTCAGCAGAATGATGAAGACA | 80 |
| SCN2A E18-I18F | TGAGGAATCCCTTGAACCTG | 81 |
| SCN2A E18-I18R | TTCAAACAGCAAATGCAAAAA | 82 |
| SCN2A E19-E20F | GGGCTCTCGCCTTTGAAGATA | 83 |
| SCN2A E19-E20R | AGGAAGTCTAGCCAGCACCA | 84 |
| SCN2A E19-I19F | CAAAGGGAAACTCTGGTGGA | 85 |
| SCN2A E19-I19R | TTGAAGGGAAAGGGAAAAGA | 86 |
| SCN2A E20-21F | CTGATTGTTGATGTCTCACTGG | 87 |
| SCN2A E20-21R | ATTCCTTCAAACCGGGACA | 88 |
| SCN2A E20-I20F | ATGCTGCTAAAGTGGGTTGC | 89 |
| SCN2A E20-I20R | TGTCAGGCACCATTGCTAGA | 90 |
| SCN2A E21-E22F | GAATGAGGGTTGTTGTAAATGC | 91 |
| SCN2A E21-E22R | ACCTGGCAGTTTGATTGCTC | 92 |
| SCN2A E21-I21F | AGGCCACTGAGAGCTTTGTC | 93 |
| SCN2A E21-I21R | GACTCCAGCAGTGCCAAGTA | 94 |
| SCN2A E22, | GTAGCCACGTTTAAGGGATG | 95 |
| 23, 24F | | |
| SCN2A E22, | GACACCAATGAAAAGATTCAAGG | 96 |
| 23, 24R | | |
| SCN2A E22-I22F | GCCAGGTGGAAAAATGTGAA | 97 |
| SCN2A E22-I22R | TTACATCCCTGCCAACAATG | 98 |
| SCN2A E23-E24F | CACGAAATGTAGAATTACAACCC | 99 |
| SCN2A E23-E24R | GACACCAATGAAAAGATTCAAGG | 100 |
| SCN2A E23-I23F | CACGTTTAAGGGATGGATGG | 101 |
| SCN2A E23-I23R | CAGAGTGATTAGAGAAAATCATGCTATG | 102 |
| SCN2A E24-E25F | GGTGTCATCATTTTGGAGGTC | 103 |
| SCN2A E24-E25R | CGAGGTATGGGTTTTTGTGG | 104 |
| SCN2A E24-I24F | CAACCCAAGTATGAAGACAACC | 105 |
| SCN2A E24-I24R | TTTGGTGATTGGATTTTGGA | 106 |
| SCN2A E25-E26F | CGACCTGCTAACAAATTCCAAG | 107 |
| SCN2A E25-E26R | TTCAGCACACATTCTCCAGTG | 108 |
| SCN2A E25-I25F | AACCCATACCTCGACCTGCT | 109 |
| SCN2A E25-I25R | AGGCTAGGTAGTCCCATTGTT | 110 |
| SCN2A E26-E27F | CATTGTAGGAATGTTTCTGGC | 111 |
| SCN2A E26-E27R | ACATCCCAAAGATGGCGTAG | 112 |
| SCN2A E26-I26F | TGTGGTGGTCATTCTCTCCA | 113 |
| SCN2A E26-I26R | CTCATCATTTCAAGCACAGCA | 114 |

Human brain RNA was used to check for intron retention in SCN2A mRNA. In order to include a wider spectrum of RNA samples, RNA from two commercial suppliers (Ambion and Takara) was obtained. As the primers span the intron, any genomic DNA isolated along with the RNA would be detected and could lead to an overestimation of intron retention. The RNA was therefore treated with DNase before reverse transcription. In addition, reactions without the reverse transcriptase enzyme were included to ensure the absence of genomic DNA. During reverse transcription of the RNA to cDNA, oligodT primers were used in order to favour the selection of transcripts that were mature (polyadenylated) and possessed retained introns. This prevented the reverse transcription of immature RNA transcripts whose introns were in the process of splicing.

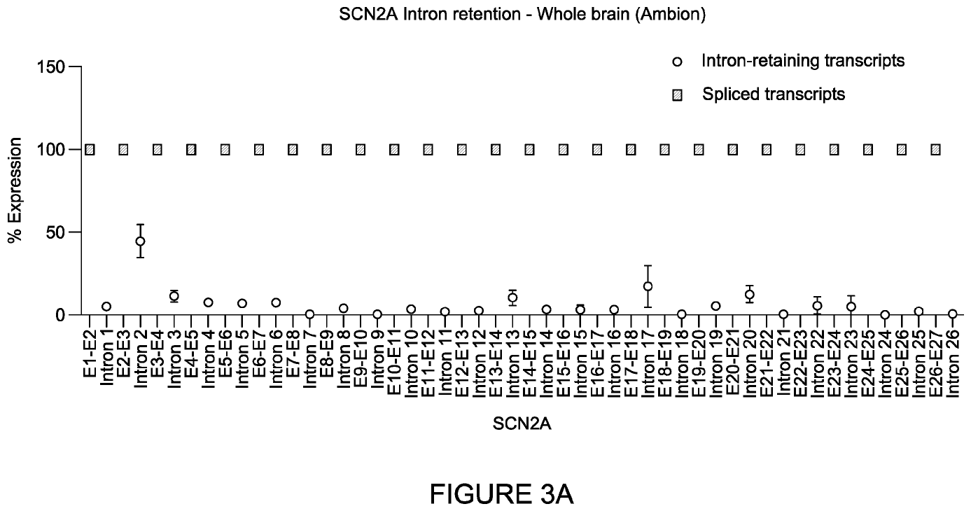

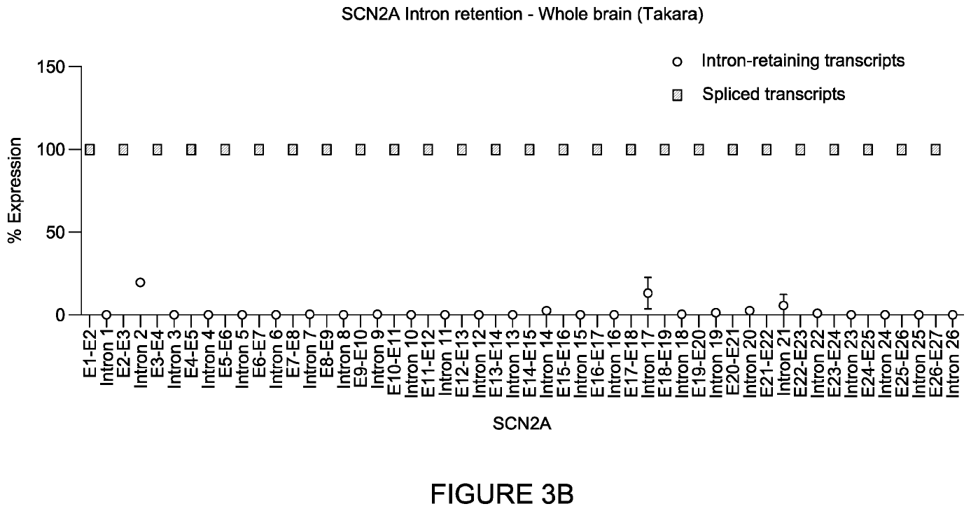

Using the strategy described above, a number of introns showing varying levels of retention in the first sample of human whole brain RNA (from Ambion) were detected, with the levels ranging from less than 10% to up to 40% of the expression of the exons. The introns showing the highest retention included introns 2 and 17 (FIG. 3A). A similar profile of intron retention was observed in the second source of human brain RNA (from Takara), where intron 2 and 17 also showed the highest retention with respect to the exons (FIG. 3B).

A number of introns are retained to varying extents in SCN2A mature mRNA transcripts. On comparison of the retention between two different sources of RNA as a representation of different experimental cohorts, there are common introns that shows retention in both the cohorts in spite of the difference in their intron retention profile.

Intron retention in two neuronal-like cell lines, SH-SY5Y and SK-N-AS, was assessed as described above. SH-SY5Y and SK-N-AS are transformed neuronal-like cell lines that were derived from metastatic bone tumours and are widely used to study neuronal function.

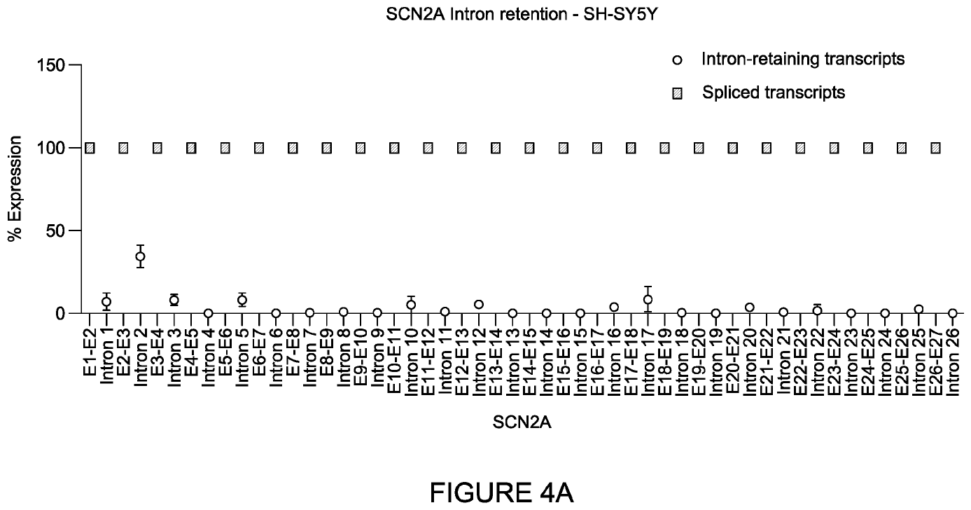

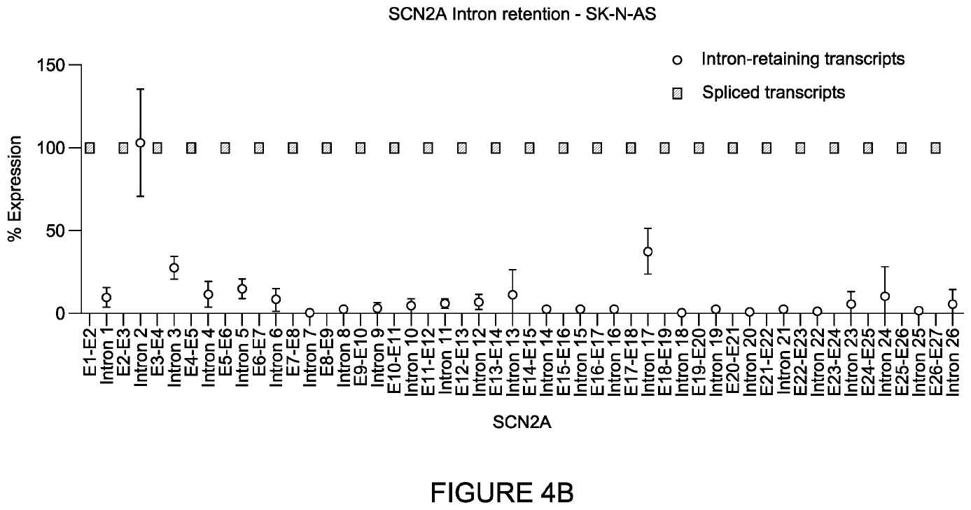

Similar to the intron retention profile observed in brain tissue, intron 2 was the highest retained intron at a level of 35% of the exon expression levels when SH-SY5Y cells were assessed (FIG. 4A). SK-N-AS cells showed high retention of introns 2 and 17, and although there was a high variation among biological replicates, the retention levels of these introns were 75% of the exon expression. The other introns in this cell line that showed retention were introns 1, 3, 4, 5, 13 and 24 (FIG. 4B).

In summary, among the introns retained in SCN2A mRNA, intron 2 (SEQ ID NO:12) and intron 17 show the highest retention levels among the different cell lines and sources tested.

### Example 3—Identification of Antisense Oligonucleotides to Reduce Retention of Intron 2

Targeting of an ASO to a particular sequence can sterically block the access of proteins, such as the spliceosome, to the nucleic acid molecule. Similarly, ASOs can be used to block sites such as splicing enhancer or silencer sequences, thereby altering the splicing propensity of a sequence. Blocking intronic splicing silencer (ISS) sites in the retained introns would in effect induce their splicing. Various tools and studies were therefore performed to identify ASOs that target the pool of SCN2A transcripts that bear retained introns and block ISS sites, so as to induce splicing and thereby increase the levels of spliced transcripts transported to the cytoplasm for translation into protein.

Although ISS sequences serve as ideal antisense targets, they are often inconspicuous in long introns. To identify the part(s) of the intron sequence that would potentially increase splicing when targeted, available prediction tools (Human Splicing Finder, SpliceAid2, RBP Map, PESXs and RegRNA 2.0) were utilised for the in silico analysis of the intron sequences.

These online tools predict splicing sites in a sequence, including exonic splicing enhancer (ESE), exonic splicing silencer (ESS), intronic splicing enhancer (ISE) and the intronic splicing silencer (ISS), based on consensus sequences. The available tools used include Human Splicing Finder, SpliceAid, RBP Map, PESXs, RegRNA 2.0.

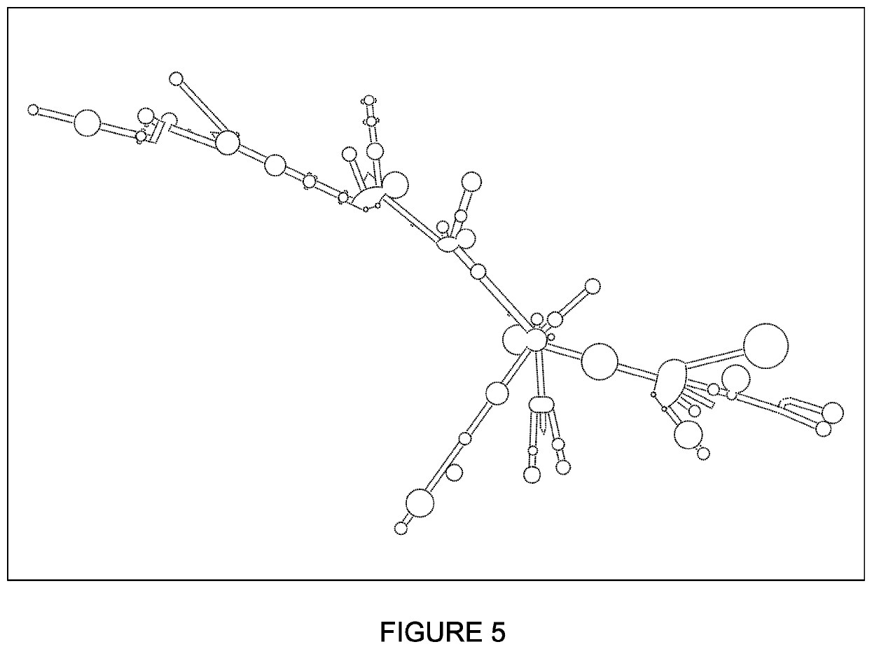

Poor splicing factor recruitment results in weakened splicing, and this has been reported to be affected by the secondary structure of the pre-mRNA (Buratti et al., 2004, Mol. Cell Biol. 24(24), 10505-10514). In-silico prediction of pre-mRNA secondary structure was performed using prediction tools such as Mfold and RNA fold. FIG. 5 shows the secondary structure prediction of SCN2A intron 2. ASOs can be designed to target regions with high propensity for secondary structure.

Based on published reports, the importance of the role of the splice repressors hnRNPA1 and hnRNP I on the splicing mechanism was inferred (Yimin Hua et al., 2008, Am. J. Hum. Genet. 82, 834-848). The prediction tool SpliceAid2 (http://www.introni.it/splicing.html) was used to narrow down the regions of the intronic sequences near the 5′ and 3′ splice sites that might be bound by these splicing silencers. HnRNPA1 sites were predicted to be at positions+8-+12 (CTTAG), +33-+36 (ATTTA) and +59-+65 (CAGGGA) relative to the 5′ splice site of intron 2, and at positions −84-−88 (ATTTA) and −82-−87 (TTTATA), relative to the 3′ splice site of intron 2. HnRNPA1 sites were also predicted for intron 17, and found at positions+21-+26 (AGATAT) relative to the 5′ splice site of intron 17, and at positions −53-−58 (TTTATA), −49-−53 (ATTTA) and −18-−24 (TTTTTTT), relative to the 3′ splice site of intron 17.

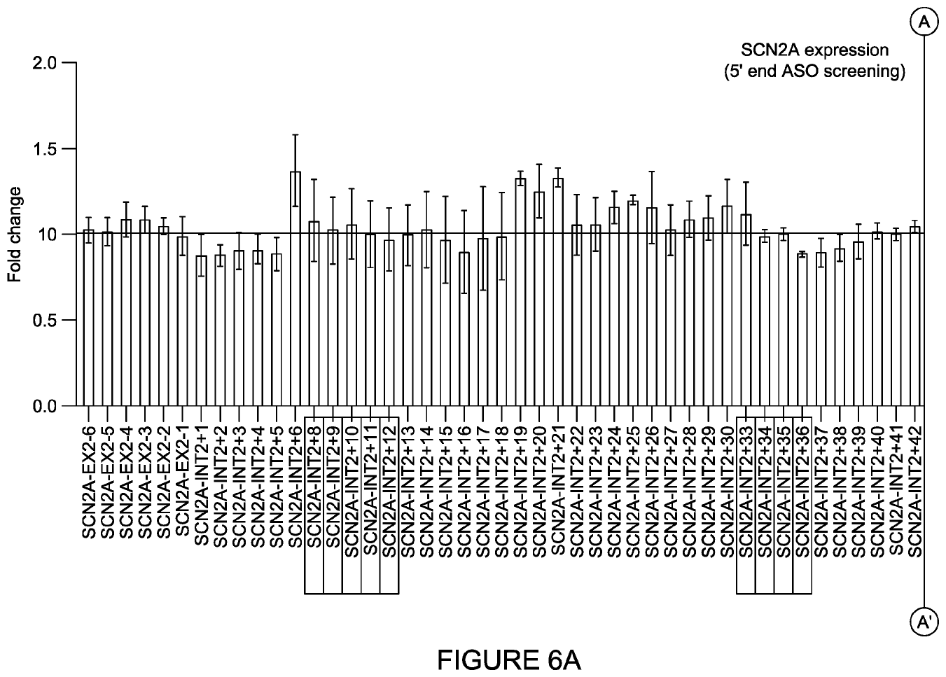

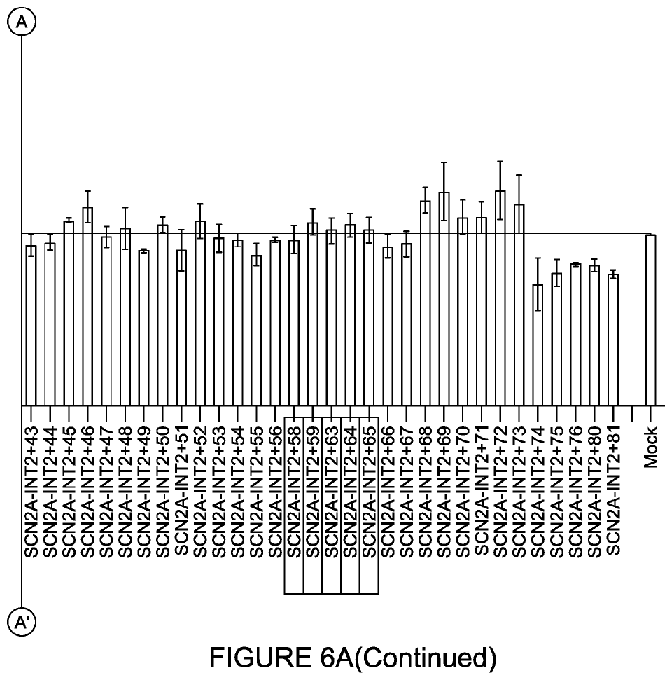

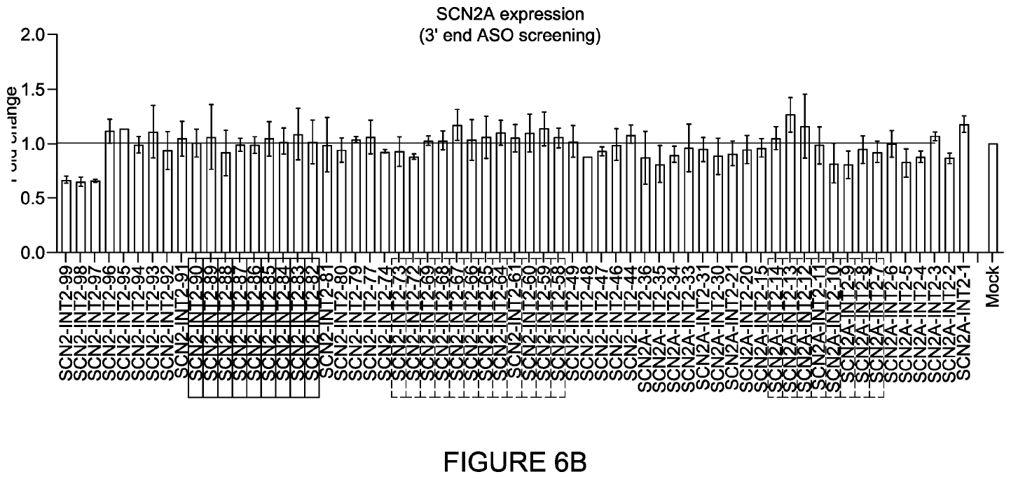

The ASOs were designed based on the predictions of the binding sites for the splicing repressors hnRNPA1 and hnRNP I, such that the ASOs would target those sites. The ASOs were 18 nucleotide-long, fully modified oligonucleotides with phosphorothioate (PS) backbone (to increase their stability) and 2′-O-methoxyethylribose (2′-MOE) sugar modifications (to increase binding affinity and reduce toxicity). A microwalk strategy was used to design the ASOs, starting from the 5′ end of intron 2 (see FIG. 6B). ASOs with off-target binding sites were excluded.

Table 2 sets forth the ASOs at the 5′ end of intron 2.

TABLE 2

| | | SEQ |
|---|---|---|
| | | ID |
| ASO Name | Sequence | NO. |
| SCN2A-EX2-6 | CTAAGAACTCACTTTCTT | 115 |
| SCN2A-EX2-5 | ACTAAGAACTCACTTTCT | 116 |
| SCN2A-EX2-4 | GACTAAGAACTCACTTTC | 117 |
| SCN2A-EX2-3 | TGACTAAGAACTCACTTT | 118 |
| SCN2A-EX2-2 | TTGACTAAGAACTCACTT | 119 |
| SCN2A-EX2-1 | CTTGACTAAGAACTCACT | 120 |
| SCN2A-INT2 + 1 | ACTTGACTAAGAACTCAC | 121 |
| SCN2A-INT2 + 2 | AACTTGACTAAGAACTCA | 122 |
| SCN2A-INT2 + 3 | CAACTTGACTAAGAACTC | 123 |
| SCN2A-INT2 + 4 | GCAACTTGACTAAGAACT | 124 |
| SCN2A-INT2 + 5 | GGCAACTTGACTAAGAAC | 125 |
| SCN2A-INT2 + 6 | AGGCAACTTGACTAAGAA | 126 |
| SCN2A-INT2 + 8 | GAAGGCAACTTGACTAAG | 127 |
| SCN2A-INT2 + 9 | TGAAGGCAACTTGACTAA | 128 |
| SCN2A- | GTGAAGGCAACTTGACTA | 129 |
| INT2 + 10 | | |
| SCN2A- | AGTGAAGGCAACTTGACT | 130 |
| INT2 + 11 | | |
| SCN2A- | CAGTGAAGGCAACTTGAC | 131 |
| INT2 + 12 | | |
| SCN2A- | GCAGTGAAGGCAACTTGA | 132 |
| INT2 + 13 | | |
| SCN2A- | GGCAGTGAAGGCAACTTG | 133 |
| INT2 + 14 | | |
| SCN2A- | AGGCAGTGAAGGCAACTT | 134 |
| INT2 + 15 | | |
| SCN2A- | TAGGCAGTGAAGGCAACT | 135 |
| INT2 + 16 | | |
| SCN2A- | ATAGGCAGTGAAGGCAAC | 136 |
| INT2 + 17 | | |
| SCN2A- | AATAGGCAGTGAAGGCAA | 137 |
| INT2 + 18 | | |
| SCN2A- | AAATAGGCAGTGAAGGCA | 138 |
| INT2 + 19 | | |
| SCN2A- | TAAATAGGCAGTGAAGGC | 139 |
| INT2 + 20 | | |
| SCN2A- | GTAAATAGGCAGTGAAGG | 140 |
| INT2 + 21 | | |
| SCN2A- | AGTAAATAGGCAGTGAAG | 141 |
| INT2 + 22 | | |
| SCN2A- | CCCAGAACCAATTAGTAA | 142 |
| INT2 + 35 | | |

Table 3 sets forth the ASOs at the 3′ end of intron 2.

TABLE 3

| | | SEQ. |
|---|---|---|
| | | ID |
| ASO name | Sequence (3′ end) | NO. |
| SCN2A-INT2-1 | CTGAAAAAGAAAACACAA | 143 |
| SCN2A-INT2-2 | TGAAAAAGAAAACACAAC | 144 |
| SCN2A-INT2-3 | GAAAAAGAAAACACAACA | 145 |
| SCN2A-INT2-4 | AAAAAGAAAACACAACAC | 146 |
| SCN2A-INT2-5 | AAAAGAAAACACAACACA | 147 |
| SCN2A-INT2-6 | AAAGAAAACACAACACAT | 148 |
| SCN2A-INT2-7 | AAGAAAACACAACACATA | 149 |
| SCN2A-INT2-8 | AGAAAACACAACACATAA | 150 |
| SCN2A-INT2-9 | GAAAACACAACACATAAA | 151 |
| SCN2A-INT2- | AAAACACAACACATAAAA | 152 |
| 10 | | |
| SCN2A-INT2- | AAACACAACACATAAAAT | 153 |
| 11 | | |
| SCN2A-INT2- | AACACAACACATAAAATA | 154 |
| 12 | | |
| SCN2A-INT2- | ACACAACACATAAAATAA | 155 |
| 13 | | |
| SCN2A-INT2- | CACAACACATAAAATAAA | 156 |
| 14 | | |
| SCN2A-INT2- | ACAACACATAAAATAAAA | 157 |
| 15 | | |
| SCN2A-INT2- | ACATAAAATAAAACTTAG | 158 |
| 20 | | |
| SCN2A-INT2- | CATAAAATAAAACTTAGA | 159 |
| 21 | | |
| SCN2A-INT2- | AAACTTAGAATATATATT | 160 |
| 30 | | |
| SCN2A-INT2- | AACTTAGAATATATATTA | 161 |
| 31 | | |
| SCN2A-INT2- | CTTAGAATATATATTAAG | 162 |
| 33 | | |
| SCN2A-INT2- | TTAGAATATATATTAAGA | 163 |
| 34 | | |
| SCN2A-INT2- | TAGAATATATATTAAGAA | 164 |
| 35 | | |
| SCN2A-INT2- | AGAATATATATTAAGAAT | 165 |
| 36 | | |
| SCN2A-INT2- | TATTAAGAATATTTTAAG | 166 |
| 44 | | |
| SCN2A-INT2- | TTAAGAATATTTTAAGAG | 167 |
| 46 | | |
| SCN2A-INT2- | TAAGAATATTTTAAGAGA | 168 |
| 47 | | |
| SCN2A-INT2- | AAGAATATTTTAAGAGAA | 169 |
| 48 | | |
| SCN2A-INT2- | AGAATATTTTAAGAGAAA | 170 |
| 49 | | |
| SCN2A-INT2- | TAAGAGAAAAGTAAAACC | 171 |
| 58 | | |
| SCN2A-INT2- | AAGAGAAAAGTAAAACCA | 172 |
| 59 | | |
| SCN2A-INT2- | AGAGAAAAGTAAAACCAT | 173 |
| 60 | | |
| SCN2A-INT2- | GAGAAAAGTAAAACCATT | 174 |
| 61 | | |
| SCN2A-INT2- | AAAAGTAAAACCATTATT | 175 |
| 64 | | |
| SCN2A-INT2- | AAAGTAAAACCATTATTT | 176 |
| 65 | | |
| SCN2A-INT2- | AAGTAAAACCATTATTTA | 177 |
| 66 | | |
| SCN2A-INT2- | AGTAAAACCATTATTTAT | 178 |
| 67 | | |
| SCN2A-INT2- | GTAAAACCATTATTTATA | 179 |
| 68 | | |
| SCN2A-INT2- | TAAAACCATTATTTATAA | 180 |
| 69 | | |
| SCN2A-INT2- | AACCATTATTTATAAATA | 181 |
| 72 | | |
| SCN2A-INT2- | ACCATTATTTATAAATAT | 182 |
| 73 | | |
| SCN2A-INT2- | CCATTATTTATAAATATT | 183 |
| 74 | | |
| SCN2A-INT2- | TTATTTATAAATATTGCC | 184 |
| 77 | | |
| SCN2A-INT2- | ATTTATAAATATTGCCCT | 185 |
| 79 | | |
| SCN2A-INT2- | TTTATAAATATTGCCCTG | 186 |
| 80 | | |
| SCN2A-INT2- | TTATAAATATTGCCCTGT | 187 |
| 81 | | |
| SCN2A-INT2- | TATAAATATTGCCCTGTA | 188 |
| 82 | | |
| SCN2A-INT2- | ATAAATATTGCCCTGTAA | 189 |
| 83 | | |
| SCN2A-INT2- | TAAATATTGCCCTGTAAA | 190 |
| 84 | | |
| SCN2A-INT2- | AAATATTGCCCTGTAAAA | 191 |
| 85 | | |
| SCN2A-INT2- | AATATTGCCCTGTAAAAT | 192 |
| 86 | | |
| SCN2A-INT2- | ATATTGCCCTGTAAAATA | 193 |
| 87 | | |
| SCN2A-INT2- | TATTGCCCTGTAAAATAG | 194 |
| 88 | | |
| SCN2A-INT2- | ATTGCCCTGTAAAATAGT | 195 |
| 89 | | |
| SCN2A-INT2- | TTGCCCTGTAAAATAGTG | 196 |
| 90 | | |
| SCN2A-INT2- | TGCCCTGTAAAATAGTGT | 197 |
| 91 | | |
| SCN2A-INT2- | GCCCTGTAAAATAGTGTA | 198 |
| 92 | | |
| SCN2A-INT2- | CCCTGTAAAATAGTGTAT | 199 |
| 93 | | |
| SCN2A-INT2- | CCTGTAAAATAGTGTATA | 200 |
| 94 | | |
| SCN2A-INT2- | CTGTAAAATAGTGTATAA | 201 |
| 95 | | |
| SCN2A-INT2- | TGTAAAATAGTGTATAAC | 202 |
| 96 | | |
| SCN2A-INT2- | GTAAAATAGTGTATAACA | 203 |
| 97 | | |
| SCN2A-INT2- | TAAAATAGTGTATAACAT | 204 |
| 98 | | |
| SCN2A-INT2- | AAAATAGTGTATAACATA | 205 |
| 99 | | |

The ASOs were transfected into SH-SY5Y cells with lipofectamine 3000 at a concentration of 200 nM, and cells were lysed 24 hours later. Reverse transcription and qPCR analysis was performed as described in Example 1 using the Taqman® Fast Advance Cells to Ct kit (Invitrogen, Thermofisher Scientific). Taqman primers that were duplexed with a housekeeping gene in order to normalize against variations induced between replicates. The expression of SCN2A in cells following ASO treatment was compared and normalised with cells transfected with mock-transfected cells.

As shown in FIG. 6, among the intron 2 ASOs tested, ASOs SCN2A-INT2+6 consistently resulted in an upregulation of SCN2A mRNA of 1.37 fold, when compared to mock-transfected cells. The other ASOs that gave an upregulation above 1-fold include SCN2A-INT2 −4, −3, −2, +6, +8, +9, +10, +14, +19, +20, +21, +22, +23, +24, +25, +26, +28, +29, +30, +33, +34, +43, +45, +46, +49, +52, +59, +64, +65, +68, +69, +70, +71, +72, +73 at the 5-prime end and SCN2A-INT2 −1, −3, −12, −13, −14, −44, −46, −49, −58, −59, −60, −61, −64, −65, −66, −67, −68, −69, −77, −79, −82, −83, −84, −85, −89, −91, −93, −95, −96 at the 3-prime end. The variation between the biological replicates might be an indication of the varied efficiencies of transfection.

In order to elucidate the mechanism of action of the intron 2 ASOs on the upregulation of the SCN2A transcript, primers that would amplify only the transcripts without intron 2 were used. These primers bound the intron 2-flanking exons, i.e. exons 2 and 3, while the probe spanned the junction between them. They are shown below in Table 4.

TABLE 4

| | | SEQ |
|---|---|---|
| Primer | | ID |
| name | Sequence | NO: |
| APDJZ44 | AGTGCCACCCCTGC | 206 |
| SCN2A | | |
| E2-E3 | | |
| Probe | | |
| (FAM) | | |
| APDJZ44 | TCTGGACCCCTACTATATCAATAAGAAAA | 207 |
| SCN2A- | | |
| E2-E3F | | |
| APDJZ44 | GGGTTGAAGGGAGTTAAAATGTAAAG | 208 |
| SCN2A- | | |
| E2-E3R | | |

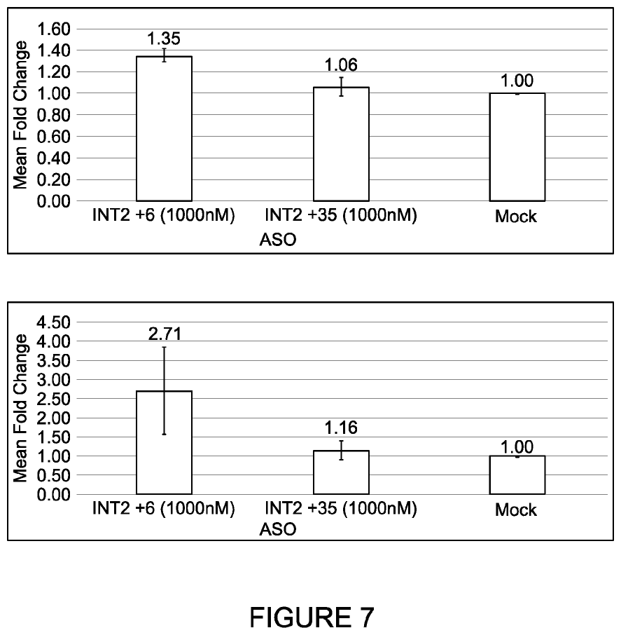

As shown in FIG. 7, cells transfected with the SCN2A-INT2+6 that caused the highest upregulation of SCN2A mRNA had increased amounts of SCN2A transcripts, as compared to the mock and to the ASO that did not cause any upregulation (SCN2A-INT2+35). This suggested that ASOs that cause upregulation of SCN2A mRNA exert their effect through the splicing out of the retained intron 2.

SEQ ID NO: 1; SCN2A transcript 2 (NM_001040142.2):
AACAGACATTGGGTACCATCGAATGACTGTCAGAACAGAAAGCTAAGGCAAAGGAGGGAGGATGCTGTGG
TCATCCTTTCTTGTTTTTTTCTTCTTTAATGAGGATAGAGCACATGTGAGATTTTACTTTCTACTCCAGT
AAAAATTCTGAAGAATTGCATTGGAGACTGTTATATTCAACACATACGTGGATTCTGTGTTATGATTTAC
ATTTTTCTTTATTTCAGCACTTTCTTATGCAAGGAGCTAAACAGTGATTAAAGGAGCAGGATGAAAAGAT
GGCACAGTCAGTGCTGGTACCGCCAGGACCTGACAGCTTCCGCTTCTTTACCAGGGAATCCCTTGCTGCT
ATTGAACAACGCATTGCAGAAGAGAAAGCTAAGAGACCCAAACAGGAACGCAAGGATGAGGATGATGAAA
ATGGCCCAAAGCCAAACAGTGACTTGGAAGCAGGAAAATCTCTTCCATTTATTTATGGAGACATTCCTCC
AGAGATGGTGTCAGTGCCCCTGGAGGATCTGGACCCCTACTATATCAATAAGAAAACGTTTATAGTATTG
AATAAAGGGAAAGCAATCTCTCGATTCAGTGCCACCCCTGCCCTTTACATTTTAACTCCCTTCAACCCTA
TTAGAAAATTAGCTATTAAGATTTTGGTACATTCTTTATTCAATATGCTCATTATGTGCACGATTCTTAC
CAACTGTGTATTTATGACCATGAGTAACCCTCCAGACTGGACAAAGAATGTGGAGTATACCTTTACAGGA
ATTTATACTTTTGAATCACTTATTAAAATACTTGCAAGGGGCTTTTGTTTAGAAGATTTCACATTTTTAC
GGGATCCATGGAATTGGTTGGATTTCACAGTCATTACTTTTGCATATGTGACAGAGTTTGTGGACCTGGG
CAATGTCTCAGCGTTGAGAACATTCAGAGTTCTCCGAGCATTGAAAACAATTTCAGTCATTCCAGGCCTG
AAGACCATTGTGGGGGCCCTGATCCAGTCAGTGAAGAAGCTTTCTGATGTCATGATCTTGACTGTGTTCT
GTCTAAGCGTGTTTGCGCTAATAGGATTGCAGTTGTTCATGGGCAACCTACGAAATAAATGTTTGCAATG
GCCTCCAGATAATTCTTCCTTTGAAATAAATATCACTTCCTTCTTTAACAATTCATTGGATGGGAATGGT
ACTACTTTCAATAGGACAGTGAGCATATTTAACTGGGATGAATATATTGAGGATAAAAGTCACTTTTATT
TTTTAGAGGGGCAAAATGATGCTCTGCTTTGTGGCAACAGCTCAGATGCAGGCCAGTGTCCTGAAGGATA
CATCTGTGTGAAGGCTGGTAGAAACCCCAACTATGGCTACACGAGCTTTGACACCTTTAGTTGGGCCTTT
TTGTCCTTATTTCGTCTCATGACTCAAGACTTCTGGGAAAACCTTTATCAACTGACACTACGTGCTGCTG
GGAAAACGTACATGATATTTTTTGTGCTGGTCATTTTCTTGGGCTCATTCTATCTAATAAATTTGATCTT
GGCTGTGGTGGCCATGGCCTATGAGGAACAGAATCAGGCCACATTGGAAGAGGCTGAACAGAAGGAAGCT
GAATTTCAGCAGATGCTCGAACAGTTGAAAAAGCAACAAGAAGAAGCTCAGGCGGCAGCTGCAGCCGCAT
CTGCTGAATCAAGAGACTTCAGTGGTGCTGGTGGGATAGGAGTTTTTTCAGAGAGTTCTTCAGTAGCATC
TAAGTTGAGCTCCAAAAGTGAAAAAGAGCTGAAAAACAGAAGAAAGAAAAAGAAACAGAAAGAACAGTCT
GGAGAAGAAGAGAAAAATGACAGAGTCCGAAAATCGGAATCTGAAGACAGCATAAGAAGAAAAGGTTTCC
GTTTTTCCTTGGAAGGAAGTAGGCTGACATATGAAAAGAGATTTTCTTCTCCACACCAGTCCTTACTGAG
CATCCGTGGCTCCCTTTTCTCTCCAAGACGCAACAGTAGGGCGAGCCTTTTCAGCTTCAGAGGTCGAGCA
AAGGACATTGGCTCTGAGAATGACTTTGCTGATGATGAGCACAGCACCTTTGAGGACAATGACAGCCGAA
GAGACTCTCTGTTCGTGCCGCACAGACATGGAGAACGGCGCCACAGCAATGTCAGCCAGGCCAGCCGTGC
CTCCAGGGTGCTCCCCATCCTGCCCATGAATGGGAAGATGCATAGCGCTGTGGACTGCAATGGTGTGGTC
TCCCTGGTCGGGGGCCCTTCTACCCTCACATCTGCTGGGCAGCTCCTACCAGAGGGCACAACTACTGAAA
CAGAAATAAGAAAGAGACGGTCCAGTTCTTATCATGTTTCCATGGATTTATTGGAAGATCCTACATCAAG
GCAAAGAGCAATGAGTATAGCCAGTATTTTGACCAACACCATGGAAGAACTTGAAGAATCCAGACAGAAA
TGCCCACCATGCTGGTATAAATTTGCTAATATGTGTTTGATTTGGGACTGTTGTAAACCATGGTTAAAGG
TGAAACACCTTGTCAACCTGGTTGTAATGGACCCATTTGTTGACCTGGCCATCACCATCTGCATTGTCTT
AAATACACTCTTCATGGCTATGGAGCACTATCCCATGACGGAGCAGTTCAGCAGTGTACTGTCTGTTGGA
AACCTGGTCTTCACAGGGATCTTCACAGCAGAAATGTTTCTCAAGATAATTGCCATGGATCCATATTATT
ACTTTCAAGAAGGCTGGAATATTTTTGATGGTTTTATTGTGAGCCTTAGTTTAATGGAACTTGGTTTGGC
AAATGTGGAAGGATTGTCAGTTCTCCGATCATTCCGGCTGCTCCGAGTTTTCAAGTTGGCAAAATCTTGG
CCAACTCTAAATATGCTAATTAAGATCATTGGCAATTCTGTGGGGGCTCTAGGAAACCTCACCTTGGTAT
TGGCCATCATCGTCTTCATTTTTGCTGTGGTCGGCATGCAGCTCTTTGGTAAGAGCTACAAAGAATGTGT
CTGCAAGATTTCCAATGATTGTGAACTCCCACGCTGGCACATGCATGACTTTTTCCACTCCTTCCTGATC
GTGTTCCGCGTGCTGTGTGGAGAGTGGATAGAGACCATGTGGGACTGTATGGAGGTCGCTGGCCAAACCA
TGTGCCTTACTGTCTTCATGATGGTCATGGTGATTGGAAATCTAGTGGTTCTGAACCTCTTCTTGGCCTT
GCTTTTGAGTTCCTTCAGTTCTGACAATCTTGCTGCCACTGATGATGATAACGAAATGAATAATCTCCAG
ATTGCTGTGGGAAGGATGCAGAAAGGAATCGATTTTGTTAAAAGAAAAATACGTGAATTTATTCAGAAAG
CCTTTGTTAGGAAGCAGAAAGCTTTAGATGAAATTAAACCGCTTGAAGATCTAAATAATAAAAAAGACAG
CTGTATTTCCAACCATACCACCATAGAAATAGGCAAAGACCTCAATTATCTCAAAGACGGAAATGGAACT
ACTAGTGGCATAGGCAGCAGTGTAGAAAAATATGTCGTGGATGAAAGTGATTACATGTCATTTATAAACA
ACCCTAGCCTCACTGTGACAGTACCAATTGCTGTTGGAGAATCTGACTTTGAAAATTTAAATACTGAAGA
ATTCAGCAGCGAGTCAGATATGGAGGAAAGCAAAGAGAAGCTAAATGCAACTAGTTCATCTGAAGGCAGC
ACGGTTGATATTGGAGCTCCCGCCGAGGGAGAACAGCCTGAGGTTGAACCTGAGGAATCCCTTGAACCTG
AAGCCTGTTTTACAGAAGACTGTGTACGGAAGTTCAAGTGTTGTCAGATAAGCATAGAAGAAGGCAAAGG
GAAACTCTGGTGGAATTTGAGGAAAACATGCTATAAGATAGTGGAGCACAATTGGTTCGAAACCTTCATT
GTCTTCATGATTCTGCTGAGCAGTGGGGCTCTGGCCTTTGAAGATATATACATTGAGCAGCGAAAAACCA
TTAAGACCATGTTAGAATATGCTGACAAGGTTTTCACTTACATATTCATTCTGGAAATGCTGCTAAAGTG
GGTTGCATATGGTTTTCAAGTGTATTTTACCAATGCCTGGTGCTGGCTAGACTTCCTGATTGTTGATGTC
TCACTGGTTAGCTTAACTGCAAATGCCTTGGGTTACTCAGAACTTGGTGCCATCAAATCCCTCAGAACAC
TAAGAGCTCTGAGGCCACTGAGAGCTTTGTCCCGGTTTGAAGGAATGAGGGTTGTTGTAAATGCTCTTTT
AGGAGCCATTCCATCTATCATGAATGTACTTCTGGTTTGTCTGATCTTTTGGCTAATATTCAGTATCATG
GGAGTGAATCTCTTTGCTGGCAAGTTTTACCATTGTATTAATTACACCACTGGAGAGATGTTTGATGTAA
GCGTGGTCAACAACTACAGTGAGTGCAAAGCTCTCATTGAGAGCAATCAAACTGCCAGGTGGAAAAATGT
GAAAGTAAACTTTGATAACGTAGGACTTGGATATCTGTCTCTACTTCAAGTAGCCACGTTTAAGGGATGG
ATGGATATTATGTATGCAGCTGTTGATTCACGAAATGTAGAATTACAACCCAAGTATGAAGACAACCTGT
ACATGTATCTTTATTTTGTCATCTTTATTATTTTTGGTTCATTCTTTACCTTGAATCTTTTCATTGGTGT
CATCATAGATAACTTCAACCAACAGAAAAAGAAGTTTGGAGGTCAAGACATTTTTATGACAGAAGAACAG
AAGAAATACTACAATGCAATGAAAAAACTGGGTTCAAAGAAACCACAAAAACCCATACCTCGACCTGCTA
ACAAATTCCAAGGAATGGTCTTTGATTTTGTAACCAAACAAGTCTTTGATATCAGCATCATGATCCTCAT
CTGCCTTAACATGGTCACCATGATGGTGGAAACCGATGACCAGAGTCAAGAAATGACAAACATTCTGTAC
TGGATTAATCTGGTGTTTATTGTTCTGTTCACTGGAGAATGTGTGCTGAAACTGATCTCTCTTCGTTACT
ACTATTTCACTATTGGATGGAATATTTTTGATTTTGTGGTGGTCATTCTCTCCATTGTAGGAATGTTTCT
GGCTGAACTGATAGAAAAGTATTTTGTGTCCCCTACCCTGTTCCGAGTGATCCGTCTTGCCAGGATTGGC
CGAATCCTACGTCTGATCAAAGGAGCAAAGGGGATCCGCACGCTGCTCTTTGCTTTGATGATGTCCCTTC
CTGCGTTGTTTAACATCGGCCTCCTTCTTTTCCTGGTCATGTTCATCTACGCCATCTTTGGGATGTCCAA
TTTTGCCTATGTTAAGAGGGAAGTTGGGATCGATGACATGTTCAACTTTGAGACCTTTGGCAACAGCATG
ATCTGCCTGTTCCAAATTACAACCTCTGCTGGCTGGGATGGATTGCTAGCACCTATTCTTAATAGTGGAC
CTCCAGACTGTGACCCTGACAAAGATCACCCTGGAAGCTCAGTTAAAGGAGACTGTGGGAACCCATCTGT
TGGGATTTTCTTTTTTGTCAGTTACATCATCATATCCTTCCTGGTTGTGGTGAACATGTACATCGCGGTC
ATCCTGGAGAACTTCAGTGTTGCTACTGAAGAAAGTGCAGAGCCTCTGAGTGAGGATGACTTTGAGATGT
TCTATGAGGTTTGGGAGAAGTTTGATCCCGATGCGACCCAGTTTATAGAGTTTGCCAAACTTTCTGATTT
TGCAGATGCCCTGGATCCTCCTCTTCTCATAGCAAAACCCAACAAAGTCCAGCTCATTGCCATGGATCTG
CCCATGGTGAGTGGTGACCGGATCCACTGTCTTGACATCTTATTTGCTTTTACAAAGCGTGTTTTGGGTG
AGAGTGGAGAGATGGATGCCCTTCGAATACAGATGGAAGAGCGATTCATGGCATCAAACCCCTCCAAAGT
CTCTTATGAGCCCATTACGACCACGTTGAAACGCAAACAAGAGGAGGTGTCTGCTATTATTATCCAGAGG
GCTTACAGACGCTACCTCTTGAAGCAAAAAGTTAAAAAGGTATCAAGTATATACAAGAAAGACAAAGGCA
AAGAATGTGATGGAACACCCATCAAAGAAGATACTCTCATTGATAAACTGAATGAGAATTCAACTCCAGA
GAAAACCGATATGACGCCTTCCACCACGTCTCCACCCTCGTATGATAGTGTGACCAAACCAGAAAAAGAA
AAATTTGAAAAAGACAAATCAGAAAAGGAAGACAAAGGGAAAGATATCAGGGAAAGTAAAAAGTAAAAAG
AAACCAAGAATTTTCCATTTTGTGATCAATTGTTTACAGCCCGTGATGGTGATGTGTTTGTGTCAACAGG
ACTCCCACAGGAGGTCTATGCCAAACTGACTGTTTTTACAAATGTATACTTAAGGTCAGTGCCTATAACA
AGACAGAGACCTCTGGTCAGCAAACTGGAACTCAGTAAACTGGAGAAATAGTATCGATGGGAGGTTTCTA
TTTTCACAACCAGCTGACACTGCTGAAGAGCAGAGGCGTAATGGCTACTCAGACGATAGGAACCAATTTA
AAGGGGGGAGGGAAGTTAAATTTTTATGTAAATTCAACATGTGACACTTGATAATAGTAATTGTCACCAG
TGTTTATGTTTTAACTGCCACACCTGCCATATTTTTACAAAACGTGTGCTGTGAATTTATCACTTTTCTT
TTTAATTCACAGGTTGTTTACTATTATATGTGACTATTTTTGTAAATGGGTTTGTGTTTGGGGAGAGGGA
TTAAAGGGAGGGAATTCTACATTTCTCTATTGTATTGTATAACTGGATATATTTTAAATGGAGGCATGCT
GCAATTCTCATTCACACATAAAAAAATCACATCACAAAAGGGAAGAGTTTACTTCTTGTTTCAGGATGTT
TTTAGATTTTTGAGGTGCTTAAATAGCTATTCGTATTTTTAAGGTGTCTCATCCAGAAAAAATTTAATGT
GCCTGTAAATGTTCCATAGAATCACAAGCATTAAAGAGTTGTTTTATTTTTACATAACCCATTAAATGTA
CATGTATATATGTATATATGTATATGTGCGTGTATATACATATATATGTATACACACATGCACACACAGA
GATATACACATACCATTACATTGTCATTCACAGTCCCAGCAGCATGACTATCACATTTTTGATAAGTGTC
CTTTGGCATAAAATAAAAATATCCTATCAGTCCTTTCTAAGAAGCCTGAATTGACCAAAAAACATCCCCA
CCACCACTTTATAAAGTTGATTCTGCTTTATCCTGCAGTATTGTTTAGCCATCTTCTGCTCTTGGTAAGG
TTGACATAGTATATGTCAATTTAAAAAATAAAAGTCTGCTTTGTAAATAGTAATTTTACCCAGTGGTGCA
TGTTTGAGCAAACAAAAATGATGATTTAAGCACACTACTTATTGCATCAAATATGTACCACAGTAAGTAT
AGTTTGCAAGCTTTCAACAGGTAATATGATGTAATTGGTTCCATTATAGTTTGAAGCTGTCACTGCTGCA
TGTTTATCTTGCCTATGCTGCTGTATCTTATTCCTTCCACTGTTCAGAAGTCTAATATGGGAAGCCATAT
ATCAGTGGTAAAGTGAAGCAAATTGTTCTACCAAGACCTCATTCTTCATGTCATTAAGCAATAGGTTGCA
GCAAACAAGGAAGAGCTTCTTGCTTTTTATTCTTCCAACCTTAATTGAACACTCAATGATGAAAAGCCCG
ACTGTACAAACATGTTGCAAGCTGCTTAAATCTGTTTAAAATATATGGTTAGAGTTTTCTAAGAAAATAT
AAATACTGTAAAAAGTTCATTTTATTTTATTTTTCAGCCTTTTGTACGTAAAATGAGAAATTAAAAGTAT
CTTCAGGTGGATGTCACAGTCACTATTGTTAGTTTCTGTTCCTAGCACTTTTAAATTGAAGCACTTCACA
AAATAAGAAGCAAGGACTAGGATGCAGTGTAGGTTTCTGCTTTTTTATTAGTACTGTAAACTTGCACACA
TTTCAATGTGAAACAAATCTCAAACTGAGTTCAATGTTTATTTGCTTTCAATAGTAATGCCTTATCATTG
AAAGAGGCTTAAAGAAAAAAAAAATCAGCTGATACTCTTGGCATTGCTTGAATCCAATGTTTCCACCTAG
TCTTTTTATTCAGTAATCATCAGTCTTTTCCAATGTTTGTTTACACAGATAGATCTTATTGACCCATATG
GCACTAGAACTGTATCAGATATAATATGGGATCCCAGCTTTTTTTCCTCTCCCACAAAACCAGGTAGTGA
AGTTATATTACCAGTTACAGCAAAATACTTTGTGTTTCACAAGCAACAATAAATGTAGATTCTTTATACT
GAAGCTATTGACTTGTAGTGTGTTGGTGAAATGCATGCAGGAAAATGCTGTTACCATAAAGAACGGTAAA
CCACATTACAATCAAGCCAAAAGAATAAAGGTTTCGCTTTTGTTTTTGTATTTAATTGTTGTCTTTGTTT
CTATCTTTGAAATGCCATTTAAAGGTAGATTTCTATCATGTAAAAATAATCTATCTGAAAAACAAATGTA
AAGAACACACATTAATTACTATAATTCATCTTTCAATTTTTTCATGGAATGGAAGTTAATTAAGAAGAGT
GTATTGGATAACTACTTTAATATTGGCCAAAAAGCTAGATATGGCATCAGGTAGACTAGTGGAAAGTTAC
AAAAATTAATAAAAAATTGACTAACA
SEQ ID NO: 2; SCN2A RefSeqGene nucleotide sequence (NG_008143.1):
AAGACTTCCATCTGCCCTCCGTAGGTTCAACTTTAATGCTTCCTACAGATGGGCAACAGGGCTATCTGCT
CAGTGCAACCCCCAGAATTCCAATTGTCTGTGCTGTCTCCACTCCATACCATAACTAAGCCTAGGAAGCG
CCTCCTGAAGTTCTTCACTGGGCTGAATTAAGAACAGGATCATCGTTCTCTATCTGGGTGTTTTCTACCC
CACTGAGAGAGGTGAAATTGTCAGAACAAAAATTTAGGAATTTACCAAATGCAGTTTTTTTTTGGCTGAT
AATTACTATTATTAACAATAATGACAATGATAAGCTTAGAAAAATTAAACACTAAACAATGGCTGCCGTA
AGAACACTGAAATGACTTTATAGTTTACAATCAGTCTTTCGTCTTTCAGGGATATTTTTGTGGACATCTG
GTGCCACATTTTAGTATGGGGTTCAGAGAATATCAATTTACCTGTTTCTTCATCATGCTATATTGAGTTA
TTTACTCCACAATAAAGGTAGACCTACTGATGAGAAATGTCAGGGCAACCTTTGTGTATTTCAGTTACCC
TCAGCAAGGTGCTACCAGCTAGCATTAGTGTATTTCTGGTGTTGGTAACAGGATCATGTGAGCCTTGTAG
ATGAGAACTCCTTGTTGCTCAGGGGATCATGGAATGCTGCTCTCTTTCAGACAGCCATCTTCGGGGAAGA
GAAGGGTCTCTGGCAGAGCTCTATGGGTCCTGCACTTCTCTTTCACCTCTCTGCCTTGTTCTTTCACTCA
TTAATTTCTTGACAATTTATTTTCCTTAATAACTCTAGTTCCTTCTGTTTATCTTCTTCTGTCAATCCTT
GCATCCATTGCAAATCCATCACCTGCTCTGAAATACCACACTGAAGGGAGGTACTCCTAAAGACCTTCCT
TCTTTTCCACTCTTGTTTTACCCTTGGCTTACACAACTGTTGAACAACTGGAGCTGAGGACAAAAAGCAC
ATAAATTCCAAAGGTCAACGGAATCACTTCTTCATAGTTTACATCTCCTGTGTCTTAAGTAGAACTCAAA
TAGAACATAAGCATTGAGAACAGAGGTCTGAGTTGGAGGTCAAGACATCGTTTACTAGCTGAGGGACTTT
GGCTTTATTTTTCTCATCTCTAAGAGGGAAAAGATAAAAATCTACTTCACAGACCTGTTGCTGGGAATAA
TGAGATAATTTATGTACAGTACTTAGCACATTGCTTGGCACATAAAAAGTGCTCAATAAATGTTATATAT
TTATAACATTATTTGTAACACCTCGTCTTTCAAGGAATTATGGGAATTATTCTGTTTGTCAGACTTTGTT
TATGGAAATTATGTCCTTTTGATGGCCCAATTTTTCTTTTCCTTTTCTTCATCTTTTGATAACATACTAT
TAAATGTCAACAATTCATACTAATTGGAACTAGTGTAATAAGCTGTCCTCCTCATCAGTATTAGCAGTCT
TTGAACATGGCATAAAAATATGCCCATTTTGGCTTACAGGTCCTTGATTCTCTAATGATAATAGTGCAGG
AAAAACATTAGATATGGAGTTCAACTTTTTAAAACTGTCTTGATGTTGGTGACCAATAATTTAAAAGAGG
TTAGCATATTTTAAGGTGACTAAAACTGTACAAATGAAATTGCCATTTAAAAATGTCTGTTCATTTGCTT
TTCTCCAATGCAGATATTTTAAATAAGAAGAAGTGAGGCAAGAAAAATGTAAACAAATATTAGGTATAAA
CTAGGAACTTAGTCTTTTATTTTCTGAATCAGATGGATGATATTTAAAAGTCTGGGTGAATGATAAATGG
TAGAAAAAAATTAAATAACAGTCAATATGCACAACTTAATGGAGGTAAAATAAGACTTACTTACTTTTAT
CTCTATAAATTAAAATGATTTTGAAAATCAAGCTGCCAAAATAAAGGTGTGGATTTTATCATTAAAGCAA
TGCCCAAGAGACATGTTATTGAGAAATTATGTGGCATTGGTTTGTTGTTACTAGCGAAATGAATGCCTAA
CTGAAGTTGATTTCATAAAGATTTAGTGTGTGTATGTACTTGTGTGTCTGTGTGTATATATGTATATATA
TATTTGTATACACACGCACACATGTCTACCATGTGACATGTTGCTATTTTTACAACTTATTTTAAAACAG
CTAAAATTTTCTAGATGAGATTAAACATTTTCCATAAAAATAAAATTACAATAGTTACCTCAAAGTTTGT
TTATCCTGGAAAAAAAAGTGCTAACATAAGAGTTGTTCAATAATATTTTACCAAAATTTTGTCAGAACTC
CATTAAAAACAATTCAAGACCACAGTGGTCTAATTTCAACACTGGCATCATTAACCTAACATATTTATGG
AATATTCACTATATGTTCTGTTTTCTGCTTAAGTATATGAATTAAACATAGTTTAAATAGTTTACTCAAA
GAACATGCCATTTGCTTCATTGATTTCACACTCTCTGACGTATGTATTAATAAGAACATAGGAATCCACA
CACAGAAATAATAGCATTCCTATGGTGCATTAATAAAAATGTATGAATTTTCTATGTCTAGGACATGTAT
TCATTATTTGGGTGGGTCTTATATTTCAGGTACTGTTCTGTGAACCACGTTTATGGAAGTGATCAAAACA
GGCAAACATCTCTGCCTTCATGGAATTTACATTTTTCTTGGAGGAGGCAGACAATAGACAAAATATTTAG
TATGTCAGCTAAATACTAAAGGGAAAAACCAGAGGAGGGGATAGTGTGTGGTATTGAGGGTCAAGGCCAG
TCTCAGTAAGAAGGCAGCTGTGAGCTGTGACAAGAATGAGGGAAGTGAGCCATACGGAGCCCATGTAAGA
GATTTCCAGGCAAAGAGATTAAAAGGTGGACGCCTCCTAAGGAATGGACATACCTGCCATGTTTGAGTAA
TGAGAAAGAAGATAAAATGGCTAAAGTAGAGTGAGTGAGGGCAAGAATAACATCAGAGAAGTAAAGGAGT
CTAGATTGTGAGCATTGTGAACACTGGGGAAGGCATAGACTATTCACAAGGAGATGGGTAGCTACTAAAA
AGTTTTGAGAAGAAGAGTATGTGATATGATTTAGGGTTGAAAAGGATTGCTCCAGTTTCTGTGTCATATA
ACCCTATGGTCTTGAAACTGGGATATTGTATCCCTGGGGGCATGAGGTGGCGTGCCTAGAGGTACACAAA
ATCCCAGGATCAGTTAGTCACATCTTCCCTAAGGATAAAATTTACCCTCAAGACTATGGGGAAAAATGAT
TACTGTGTTAAGAAAAACATTTAAATTTTATATAAATTATTGTAAAATTCCCGTGATTTTAAAAATAAAG
TATCAATAATATTTCATATTTGTGGCATCTTAGGCTTTAATCTTACAGGTTTAAGCTTCTTGGCCATCAG
GAGTAATTTGTTGAAATTGCAAATTAATTTGGCAAAATTATCATAATTATGTGCCCTGAGAGCTTATGTA
TCTTATGATAGTGCAGGAAACTTGTGAGATGAAATAGTTGAGTGTATACATTAAAATAATTTGATCTACC
AAGTCACTTTAATTATTTATTTGTTCAGTGTTAAAAATATATTTTCATAACCAAACTAATACATCTTAAT
AATGTTGTAGAAATGGAAGTAAGGTAAAATTTAAGAAACTGGGTTTGTTCTTCTGACTTGCTTATTAATA
CTGGATAAGTTACTGAAGAGTGGGCTTTGGTTTCCTCGTCTATTTAATAGTGTGGGAACAACCTTATGTT
AAAGCAATCTATTCAGTATAACTTTAGCACACAGAAAATAAAATATTATGAATCCAAAGAAAGTGTAGCT
TGCAATCATGTATTTTATTAAATGTATTTATCAGCATTTTGGCTTCTGATGGACATGTGTATATACATAT
TTTGTGGATAAAAAAATTACTTAACTATTTTTATATTAGATACTATCAATTATTCTGGCTGATCTGGGGA
CTTTTAATGGTAAATTTTATATCAAATCTGACAAAGACAGTATTTCAGACCCGTTTAAGACCTAGAACTC
ACCATGAGTTCTAAAATTGGTTCTCAGCACCATGGACAGCGTTACTGCAATAGGAAATTAAAGATCGATT
TGGCCCCAAATTAAAATGGTGTTGTAAAAAAGTGGGAGAAAAAAAATGCCTATCCTTTTACTTCAAATTT
TAAAAAAATGATCCTGGCCTTCACAACTGTTCATAAGAAGAATAATTAATTAAACAAACATATATTGAGA
ACATCATATGCTCAGTAAAACTTTGATTCTATAAATGGTGTCATTTACCAAATGGATTCTTTTGACAATT
TAATTTTCTCTTATCTCTCTAAGAAGATGTAACTACACACTATAGTATACTACTACAATTATCAAATTTC
ATGTTGCATGTAACTTGTCGTCTGTATTTTTGTAGTTAGATTAGATTAACTAAAGATTTTTCAAGTTTGC
CTTTAAGTCATTTAATTTTCCTGCCTTATCTTTAACCTTTCAACATTCCTCCAAACAATAGCAACACAAG
TGTTATGTGTTAACTTCTCTAGTGACAAAAACTTATACTTCTCCACAAAGAGATGTGATGTTCATTATCA
ATAAGCTTGACATCTAAAATTGTTTTATAGGAGATACATATTACTTTTTCAGATGGTATATAAAGTTAAA
TAAATCTTAAGTTTTCAATGATGGGAAAAGCTTCCATTTAGTTTAAACATAATGTAAAGAAATTTGAATC
CCCAAAATAGAATTATAATTCTAAAAATTCATACTATAATTCTTCTTAAATGTTTAAATTACAGTTAATT
AAAGTAGTTGATTTCAAATAGAGTGGAATTATGGGCTGTACATCATTTAATTTTATGTGCTGACTGCTAC
ATAGCCAAAGGAACGTGAATTAAGATGGTTCCACTTTTGACCAGGAGATGGAGCTGTCATGTAAGATGCT
GCCTTTATTTATTTATTTTTCTAATTTAGCATGCTGTTTTCTAACAGACATTGGGTACCATCGAATGACT
GTCAGAACAGAAAGCTAAGGCAAAGGAGGGAGGATGCTGTGGTCATCCTTTCTTGTTTTTTTCTTCTTTA
ATGAGGATAGAGCACATGTGAGATTTTACTTTCTACTCCAGTAAAAATTCTGAAGAATTGCATTGGAGAC
TGTTATATTCAACACATACGTGGATTCTGTGTTATGATTTACATTTTTCTTTATTTCAGGTAAGCCAGCA
TGATTCTATTTTTGACTTATCCACGGATTGTTATCTATGTTAAGAATGACATTTAATATAAGATGTGTGC
TTTGTTAGCTTGTATTCAGATCTAAGAGATTCAAAAGCTCTAATTCTAGCTGTTGTGCAATTTAAAATCT
TCCTAGGCTGAAATGAGCTCTGACTATGACATACCGTGTTTTATTATTTCTTTGGCTTTCTAGCTGTTGG
TCTCTGTCTCTGGCTGTATTTGTTTACCTTTTAAAGGTAAAGCTTTCAAAGTGAAGGATGGGTTTTCCTT
TCACAAGGGAAATACTGCTCCTTAATGCAGTGAACAAATTGTAGAGATATAATGAAGCAGATTAACATGG
CAGTGGCTCTGCCAAAGCTCAGGGCTGAGCCTTCTGATTGCAGCCTCAGAGGCAAGCCAATATTCTGAGT
CCAAAGGCCTTTTGAACGATTTCTGCTGGCTATGATGGTACAGATTGTTTATGTCTTTATTTTTACCTCT
TAGAAATAATACGTAAGTCATTTCTCCCACATGAATCAAGATTTATGTAATGTGTTTAATAGACTTGGAA
TGAGACTATAGACCCTCCCTCCTCCCCCAGTATGGATGTGAGGAGTATGCTGAACACAAATTAATTGCCA
TGAAAATTGATTTTTTCTGTATTTTATTGGATTTGGGATAATAGAACCGTGGCCATTTTAAATAGTTGAT
ATTATAGAAGGTGCTCACATTTTAAATTATTTTAGGTCATGTGACATTATCTGTGTTAAAGAGTGGAATG
ACTCAGGAGGTGGAAGTTGCATGCAACAGCCTTGCTTCTCTCTGCTGCTTCTCGTGATTTCCCTTAAATC
CTTGCTTGATTATTATTAGAATAACAGGAATAAAGACATATAGAAATCCTATATTTAAAAACCACTGATT
TTCATTTAGAGAAATTTAAAGAGATTTTACAAATATTTTAAAAATAGTTTTGTAAGATTGTTTTTAAATG
CCTCACTTCTTTCTCCTGTGGAAAGAGCTGTGTGTGTGTGTGTGTGTGTGTGTGTGTGTGTGTGATGGTG
GAGGTGGCAGGCGGTGGAGGTGCATCAGGATGTTTTTATCTCAGTCATTGTTTTCCTGTAAAGAGGTAGT
TTTGCCATGTCAGAGGTTGTGAAAGCTATGGGGAATGATTTTATAAAATGGTTTGGACAAACCATGAAAA
AAACATGTTTTTTAAATGTGTGGAATCTATAGAGTTTGTAGTTCTACATGGCTCCATATACATGGAGATT
TTTAGAATTACAGATATTTAACCATTTATCCCCATTATCATTTAAAGAAAAGCAATTGTAATTGAGAGAT
AAGGTGGATTTCCCAGAGTCACGTACTAAATTACAATAAGGCAAGGCTAGAACAGTGATTGACAGTTCCA
CATCTTCCATAGCAGTAACACAATTCACCTCTAGTGTGAACATATCAGGATGGCATAGACCAGGTATGAT
GATATTCACTGTGCGTGTGTGTGTGTGTGTGTGTGTGCATGTGTGTGTGTGTTTGGTTATATTCTGTAAA
ATAGAGTTTCTTTCAGTATACTGTTGCTCAATGGCATATTATATGCAGTGCTCAATTTTCTTAGGTTTCT
TGTCAAAGCATCCAAGAAAACTGAATGACAACTATGGAAACTCTGTTTCTTAGTATGCCTTTTTCATTAT
TAGGTGGTGTATACTCTTCTACCCTATGCTCATGAATGATGTGGATGGCCACATGGCTTTCCCTCCTACC
TAATAGAAAAGATTGTACATATAGGTTAATCTATTGACAACTATAAGAATATCATCTCATGTCCAAGCCT
TTCCTCCTTTGTTCACTTACTAGGCACAGGCTTTACCCTACTTCTTCAATATCTTGAGAAAAGAAAGGGA
TTAAAATGTGTCATGTCTACATATATTTGTGGAATACCACGTACCAACCAAGTGTCCATGGTAGAGATAA
AGAAATAATTAGTCAAGGAGTTTACCTCAGGGAGGTTAACATGCAAATATACATGTGCAGATACCACTAT
ATCTCAAAATAAGGAATGAAGAAAGATGAGAAAGTGAATGGGACAGGAATATCAGCACCTATCTTGTGCC
AGGCACCATGTTACATCCTGGGATTCAGAACAAAACAATAATGTTAATTTCTGTCCATGGGGGGCTCAGA
GGAAAAGATTTTAGGAGGATGTGACAGTTAAATTAGATCTTTAAGAGTGAGTTGGATATAAATAAAGAAA
AACAGAAAGTTGCTTATTCCAGGTACATAGGTGAGGCAAGTAAATACAAGAAAATGGAGAACTATAGGCA
TCGTATAGGGAAACTGTAGAGAAAGGTAAATAACCCAATCTGTTTGAAGCTTTTGTTGTCGGTGTTGTTG
TTGCAGTTGTTATTTTTTTTCCCCTGGGGCAGTAAAAGTAAGGATAATGAATTTGGCTTTTGGTGTTATG
CATTTGACATACTGACACATACTCATACACTGGGGTGTTTTATAAGGAGCTGAACATGTTATATCCATGT
ATCAGATTAGTGGTTAAGGCTGAATGTATAGATCTGCATGCAGTTGTGGGACAAAATTAGGTCATACATA
ATTAAACATTAATGGGGAACAAAAGAGAAGTGAAGATGACTGGGGGCATAATCTTAAGATTAGGAGAGTA
AAAAAGTGTAGAAAACTATTATTAAATTGTGTGAAAATAATGTGAGAAGTTGACGGAATAACAATATCAT
AGGAGGCCAAGCAGGTAAATTGAATATCAAAATAGGGATCAGAAACAATAGAAAACCCATAAAGGGGTTG
GTTCATAAGAGGATAGAGTAAAGATGATGGATTTAGTTATCATTATTAGGTCATAAATTAATTTTAAGAG
AAGAGGTTTAGTTGAATGGCAGAGACAGGGGCTCTGAGGACTAAATTGGACATGAGAAATCTGGGGAGTA
TATTTAAGTTTTAAGAAGTTTGCCAGGGAAAGGTAGGAGAGATAACATTTTATACCTGGAGAAAGAAGGT
ACTGTTGAGGGAAATTTAACTTTAGTGCAGATTAGAAAAACCTAAGCCCTTTGTAGAGTAAGGTATGAAG
TCAGTAGAGAGAAAATTGAGGAAAGAAGAAACAAAAGTACACCAAGGTCGTGGAAGAGACAGAAGGAAGG
TTTGAAAATGCTTTTCCTTGACACAGGAAGAAAGGGTGGTTGATGATAATGAGCAAAGCTGGTGTAACTC
ACTTTGGGTGGCCTGTTTTTCGAAATTAAATCCTTGTTTGTAAGATGAAGTATCTCCTCTGATTAAATCA
GTTTGTTCCTCAGTAAAGGCTATTGGTGGCATATGGACTTTCTGGAAGAAATAATTAATCCTCAGTAGCA
AAACACTTTCCTGCAGAATATATTTTAGTCACATTAATTCTTGAATTGGAGACTAATAACACTGTTTCAC
ATGAAGCTTTTACATACTTCAGTTATCAATTTCTTATTCATAACCACCTATTTAAAAACAGGTTCTATTT
AAAACTTGTATTGAATTAGGCCAACACATATCTATTATACATATCTAGTTTGATTACTAAGGGCTGGTTT
GCCAGTATCTAAATTAATTATTCAAGTTCTTTCCTTTTTTGAATCCTTCAGTTTCATTAGCACGTTCACC
AGGACTTGTGTAGAGAGAGTCAATGTCATGGCCGGGTGCAGTGACTTATGCCTTTACTCCCAGCACTTTG
GGAGGCCGAGGTGGGCGGATTGCCTGAGGTCTGGAGTTTGAGACCAGCCTGGACAACATGCTGAAACCCT
GTCTCTACTAAAAATAAAAAATTAGCTGGGTGTGGTGGCAGGCACCTGTAGTCCCAGCTACTCGGGAGGC
TGAGGCAGGAGAATCGCTTGAACCTGGGAGGCGGAGGTTGCAGTGAGCTGAGATTGCGCCACTGCACTCC
AGTGTGGGTGACAAAGTGAGACTCTGTTAAAAAAAAAAAAAAAAAGTCAATGTCAGGAAGCTCCTTTCCC
TTAGGGAACTAGCTTCCTTATGAAGCTTCCTACCAGAAGTTCCTTTTTTCTCCATTTCTTCTGGCAGTTA
CAGAATCCTCTTGGGGCTTTCTTAGAGCCAATCTCCTCTAAGGTGAATGCATTTTCTTGCATTCACCTGT
CATGAAATGGCAGTGGAAAGACTTGAAGAAGGCACAGGCGTTGGTGAGTCATATATATATTTTGCTGATA
ATAATGAATACCATTGGCAATATAATAGTGGCCATTGAAATATTATTTCTATTAGGAAAAGATAACTTGC
ATCAAGAATATGAGATGAAATTCTATTAAAAAATGAAAAGTAGATGGGCCACCGTGGCTCTTGCTTATAA
TCCACTTCGGGAGGTCGAGGTAGGCAGATCACCTGAGGTCAGGAGTTCTAGACAAGCCTGGCCAATATGG
TGAAAACCTGTCTCTAGTAAAAATACAAAAATTGGCCAGGTGTGGTGGCTGACGCCTGTAATCCCAGCTA
CTCCAGAGGCCGGGGCAGGAGAATCACTTAAACCCAGGAGGCGGAGGTTGCAGTGGACTGAGATCGTGCC
ACTGCACTCCAGCTTGGGCGATAGAGGGAAACTCTGAGTCAAAAATAAATAAATAAATAAATAGAAAAGG
AAAGGAAAGAAGAAAAATAAAAGCTGACCTCTCTATAACACTCATACGTTAGTGTTATCTCCTCTTTATC
CATTTGGGTTTAGTGAAATTGGAGGTCAGTGGAAGGAGAGGTAGAAGTGGATTTTGCAAATCAGAAGTAT
AATTTTTTTTTCTGGTAATGTTCCATTAAACTTTTCAGTCAAAACCTTTATGTGGAAGAAGACATAATTA
CTAACTACATTAATAATAATAATGAAAAAATCTTATCATGTAATATCGTTTTGTTTATTAGTGAAACATT
TTAGAGAAATGATTTAAGAATCAAAGGTATTTAAGTATATTTTATTTATTGGAATGTGATAAAATATTTT
TGAGGGTAAAAAACTATAATTTCAGTTAACTGTTTGTACTTACTAAAATAACATTTAGGGGGATAAAATG
ATCTTAGGAAGAAATTGCCCACAAAAAGTGTTTTGAATTGTTTAGATTATTTTTATAGTTTTAAGTTGTT
AAGTCTTTTGCTGGTATCTAGGATTAGTTGGGAGTATAATATGCTTTGAACATGAACATGTCTTGGTGTT
ATTAGCAAATAGGTATACAATTATGTGATTGTGTAATTACTCAGCTACATCTTGTGTTGTGTGATATGTA
TGGGGAAATGACAACAAAATAACAACAGTTCTCTTAAGCATATATGAAGGTAGTGCTTGAAATATCATGA
AAAAAAGAAAGGGAGATTATGATAATGTTATGATAAGAGAAGACTGACACTATCCCTCTTCTCTGAATAA
CTGATATCAAAATGTGAAATTCTAAGGAATGAAGCAAGCTGGTCAGGGCAATGGTGATGCTGTCTCTGTA
TCAGCCCAGCATTTCCAGCTAGGATATTTTCCTTCTTTTCTGTGTGATACAGTTTGGCTCTGTATCCTCA
CCCAAATCTCACCTTGAATTGTAGTTCCCATAATCTCCACGTGTTGTGGGAGGGACCCTGTGGGAGATAA
TTTAATCACGGGGGTGGTTACCCTCCTGCTGTTCTTGTGATAGTGAGTGAGTTCTCACTCGATCTGATGG
TTTTATAGGGGTTTTTCTCCCTTTGCTTGACACTTCTCTGTCCTGACACCTTGAGAAGAAGGATGTGTTT
GCTTACCCTTCCGCCATGATTGTAAATTTCCTGAGGCCTTCCCAGCCATGCAGCACTGTGAGTCAATTAA
ACCTCTTTCCTTTATAAATTACCCATTCTTGGGTATTTCTTCACAGCAGCATGAGAACTGACTAACACAC
TATGTCTCCCCTGGATTGAGCAAATGAGGGAGCCCAGCAATTGTCAGGCAGCTAGGATCTGTGAGGAGTG
CTTAGACCTACACTTACTATTTCCTGAATTAACAGTCTAGAAGACAGGAGCCTAGTTATACAGACAGGGT
GACCATAAAATGTGTCATCTAAACCAGGATTCAGTTTGTTAATAATTTCTCCACAACGACAGGTATAAAT
CAAGATGGTTCCAGGTAAACTGGGATGCACAGTCACTCCACTTCTAAGGGTCAAGAAACTTCTACATTTA
ATCAGTCTATTCAATTAAATTGTTCATGATTTTGTTGCCTAAGTGTTAGAAAGTTAAAATGTGAAATGAC
CTATCAATTAGACAAAGGGCATTCAGCTAAGCTGCTCCAATTACTGGCCTTGATAAATCCATCAACCGAA
ATCTCTAATGATTCTTGTTGAATCCTTAGTTATATCACCAGGAATGATGTCACTCCAATAATGTTAAGCC
TGACTTCCCAGGGAATTCAACTGTGTGCAAAAAAAAACTGTTAGTAAATTATGCTGTTATTACAAACCTA
GACTCACAGCATCATAAATCTATTCAAATATAAAAGCAAAAGCAGGATAAAATATGGATTCAAATTATTT
CCACATTTGGGTATGCATCTTTTAAGGAGTTTTCATGAATGTGTAATCCTTAGAAGATAGGAATTTAGCT
CCAAGAACACTGGAATTTTCTCCACTGGCACTTTATACCCAAATCTTGGTAATGTCAATATGCATATGTA
TGGCCTCTTAACAGACTATCCTGTAAAGTAGTTAAATTATTTTCCTCCAATGATCATGTTTTTTTTACCC
TTCTTCAGACATCCACTCCCATGGTTTTATCTTGAAACTCTTCATGGCCAGTAACTGTGGCCTGCCCATA
ATCCTTCCTTCATACATCCTACACCACCTTTCTAGCGCATTTCATCTACAACTCCAATTCATTCCTTTAA
TCCCACCAGGCCTCTAAATCACTGATCTCGTCATACTTTGATTATCTCTCACTAAATGTCCTCTTTCCTT
TCATTAGATAAATTCCACAGTTATTCATAAAAGTCACTCCTTGTATATACATACATACCTCACAAACTCC
TCCGCTCAGTGTTTTTTTGTTTGGTTGGTTTTTTGGTAACTGTCTGGCTAAATCTTCATCCTCATATTCA
GTTCCCACCTTACTTCCTGCCTGCACTTTTGCAGCTGGACGCATGCAAAAGTGGGTCCTTCATGATGCCC
AGCAATCATGCCACATGTCCCTAGATTGATGCCCAGCAACCATGCCACATGTCCCTGGATCACTTACTCT
CCCAGGTTCTTGTACTTAGATTTCATACCCTTTCTTTTCTCCTCAAACCACCAATACCTCCTACCTTGAC
CTCACTCTCAGTTGACAACTTTGTTTCCTAAATCAGTGAGAAAATTGAAACAATCAGGAGAGATTTTTCC
CCACAGGTTTCTATCACTGCATGTGCCTTCCTACCAGGTTCTGCACCCACATAACCTGACCTCCCACCCA
TTACCTTAAATCAACTATCCCTGTTTCTATCTAAAATCATTCCTGCATCTGTGCACTAGATCCTATTCTT
TCTTACTTACACAAGGGCATCTCTAGAGCATTTTATTTCTCTGTCTCTCCTGCATTAGTAATTTTAGCTC
TTTATTATATTTGCATACAAGCATTTTATTATTGCTCTTGTATTTGAATTTTTTTACTTCCTTCAACTAT
CTTTTTTTTATTTTTTGTTGTCTTTGCAATAATACTTAATGAAAGTGTTTGCTGTATTCTCTGTATCTAT
GACTCTCTTTCTATTCTCTTTTAAATTTACGTTAGTCAAGTTTTTACTCTTACCATTTCACCAAATCAGC
TCCTGATAGAAATCTGATGACCACCCTATTGCCAAATTCATCAGTCCATCCTCAGCTTTCATCTTACTTG
ACCATCCAGCAATATTGGAGACAGTTCATTGCTGTCTCCCCCTTGATACACTGCCTTCCGTTGGATTTCA
GGATAGCACACAATTTTGACTCTTCTCTTAGCTTATTGTTCACTCCTTCACAGTCACTTTACTCTTTACT
CTGCCTTCCCAGACTTCATTATTTGGGTGTCCCTGATTCTGTCCCTGATTTTCTTTTCTTCTTTATCTAC
TTTCTTTTGATCCCATTGAGTTTTATGGCGTTAAATGCCATATAAATCATGATGACATCCAAATATTTAC
CTCCAGTTTTCTGCCAAACTGAACTTGTGTGTCTAACTGCTTATTTGGCTTGGATATCTAGAAGACACTG
AAAAGTTAACATCCCTAAACTAAATTTCGGATAGTTCTTCCCAACCAACCTGCTCCAATCCTCGCTTTCC
CCACCTTACTTTTTTCAGGAGATAACTCTAGTTTTCCTCATCCTCAGGCTAACAACTCTAGTATCATCCT
TGATTATTCTTTTTCTCTTATATCTGACATTCAGTCCATGAGAAATTCCTGTTGATTTCAACTTCAGAAT
ATACCCAGGATCTATGTGCTGCCACCTTCACACTGCTACCCTGCTCAAGCCACCATCAAGTTTCTCATGG
ATTTCTGCCTTAATTTCCTAATTGGACTTTATTCTTCTACTTCTCCTTACAATCTATTCTCAATACGGCA
AGACAGAAGTAAGATTACGCCACATCCCTGCTCAAAATTCTGCAAGTGATATCCAGTTTTCTCAGGGTAA
AAGGCACCTGACAATTTCCAAAAAGTCTCTATTTAGCCTGGTCTCCATCCCATTACAGTATTTCTCTGAA
TTTGTCTCCAGTTTTTCTTTCCCTCATTCACTTTCACTATTTCTTGAATATTGCAGGAACACTTCAATCC
TTTGGGCTGTTTCCTCTGCCTAGAAACCTTTTGCCTCAGGCACTTGCATGACTAACCTCCTCACCTGTTT
AAAATCTGAGCTCAAATGTCGTTTCTAAATAATACCTACTCTTAACTATCCCATTTAAAAATCTCAGCCC
CCTTCCTCTAACTTCATTCTTATCGTCTCCCACATTCTTCTATACTTTCTTTTCTTTGTTATCATTTGCT
TTGCTTTGTTCTTTTTTTCCTTTTCTTTCCTTTCTTTTATTTCCTTCCCTTCTAACACATCACATTATAG
CATACTGTATACATTGCATTTCTCTTGTTGGTAATTTAAGCCCCAAATGGGCAGAGATTATCATCTGTTT
GGTGCACTGATGTTTCACAAATGCTTAGAACACTTCCTGATCTATAGAAGAGACTAAAAATGTTTGTAGA
ATAAAATAATGAATCTTAGAAACTTAAATTTATTCCTTTAAGCATACTAAGGGGCACCATGCTTTCTCTT
TCACTTTATTGGGCTGATTCACATTTACAAATGACCCAGTGCAATCTGCCTGATTTTTCATTCAGTATTT
ACACCAATAGAAATAAAATGCATATTAGTGTTATAAAATCAAACCTTAAATGACAGTAAATGTATCATTA
ATAGTTACTGAAGACCTTTTCCTGATATATATGACATTTAATGTTTTTCTCTTGTGACTTCATTGTTCTT
TGTTTGCTTGTTCATCTTCTAGATTGTGAGCTCCTCAAAGTCAGGAACTTGGTCATCATATCTGTCTCCC
AAATACCACACTCAATGTCTGGCACATAAGAAGTTTTACAAATATGCTGTTTTAGTCATAGGATGAGCAT
ACACTTAAAATTTAATACTGGTCCTGCATATGCTGTGTCCTGAGCTTTCGCTATGTAGGTTGCTTTAATT
CTTACAACCGTGTGGAGCAAAGCACTGGTCTCATTTTACAGATGTGATAAAGCTAGTTATTTTGTCCAGG
ACTTAATCATTGCAGTATTCTCACTGTCTAATCTCTAGTGACCATATCACAGGAAGTTAATTATGCTCTT
TTTTTAAAGATAAATTGAAATAATGCTAACAATGCCAGATTTCTTGGAAATGTTTTGTTTTTAGTTTCAG
TTAGAAGCACGCCTTAGCTTAGTGAATAAGGGACAATGAATGTTATTTATTTTCCTGCTCAATTCAGTTG
GGAAAATTGACCTCATCGTTTTGTGTTTTCAATCCCTTAAGTGTAATAAAATTATTTACTACCTTAGTTT
ATATAAATCACCAAGATCTAATGGAAGCCACCCAGTTAACTCTCCTAAAGAGTCTTCAAGCATGTTTTGT
TCTAAATCTCAGCTTAAGTATTTTCCATTGTTTACCACCTAATGTCTATACTACTTGATTTATAGAAAGT
CTTATCTAATATAAATATGCTAACTAGCTATGATAAAATTGATCACTTTCCACATAATCATATACTGAAA
AAAAATTCCACTGAATATATTTCTGCCCTCCATAGATTCCAGGATTTCAGTGAATTTTTCTTAATAATCT
CTGCTTCAGAAATGTATATAAACTTTTGTCCTAATCAAATCTAACTCAAACGTCACTTCCTTTATAATGT
TCTCTCTGTTTTCACATGCAGCATATACTTCCATTCTACTCTGCACCTATCTCTACACTTGTAATGTGAA
GTAGCCAGATATATGTGGACCTGGGTCATCTGTCATATTTAATTGTGTACTATATAAGGACAGATATTGT
TTTGTATGGTTCTATCAAGCTACCCAGTATCTACATATACTGCCCTCCATAGGAGGTAATACATATGCTG
AGCTCTACTCATTTAAAACTTCAATCAAGGTACTATTGGAATTTCTTAGTTTTACAACATAACTATGTCC
TACTTCAAGTGACCTATTAAAATAGCTCTCAATAGTTTGCTTTAATACAACTGCCTTTAATAGCTTTTAA
ATTTAGTGTTTGTTTGAGTAGACTTTATGGTGATTTGTAAAATGATATGGATATAGTATATAGAAAATTT
TAACAGAAATGTAAATAATAATTATAATTTAGGGAAACGGTGCCCTCTTGCTCTATTTCACACACACACA
CACACACACACACACACACAGGCACACATATACACACAAAAAAAATCTCTATTTCCAAATCTTGTTCACA
AGGAGCTCTCTAACTCCATGAGTAATTTTTACAACCAATAACAGATTAACCAACAATAGTCACACGAACA
TGTACACACACATATACACAGCCTAACTTCCCCACATCATTATTATCTATCTATCGTCAAGAATGCAGCT
TATAAAATACAATGACCTGGCTGCTATAGGAAAGGATAAAATGTAGGTTGCAGTGATCTTCTGACATTGA
GATGGCAAGGAATGTGTTTTTTGCAGGAATTTCAAAACCTGAATTGTAGAATGATTTGAGATACTGCCTT
AACTACCAACAAATTTGATCCCATGTTCTTCATCATTTTTCTGAGATTCTGGTTTCCCAGATTTCTGACC
TTAATTGACTCAACCAAAAAAATCTGCTTATTATTAAAGACCATAAGGAGTAATCAACAGAACAAAATAT
GATTTAATATAATATTAAATATATTTTACAACTCAAAACATTCTAAAAGATGTGATGGTTTCTGACTATA
ATTCAGTATATACTATTTTAGTTTTAGTGACAATGTTTCTAACAAATACAGGAAAACGTTTCAGAACCTA
GTGTCCTTTACAGCAAGTAGCAAAGATGGACTCAGGAATATGTAAGCAAAATCTAGCCAAACCAAACCAA
CCAACCAGACAACAACAACAAAGTGTGTTGCTGAGTGGCTCATAGACAAGGACAAGTATTTGTGATGTTC
TAATATAATAGGTGGTAAGAACCAATGGAACATCTGTGATTCAAGTGAGAGTCGTGGTGGTAGAAAACAA
AACAAAAGAAACGATAATATGTGTAGAAATAATAAAAGAAAAAAGTCAAAGGGTGTATTTTTACAAGTAC
ACGTTCAAATATAGCGTGATGATATTTTATCTCAGGAATTTCATGCAAAGTGCTGCGTATGTTGATATGT
TAGGTCTCTTCTGGCCCAGTGACGATTATCTTGTGTGGTTTAAGTTCTCTATGCCATGAGTGAGGTAAGG
GCATTTATTCCCCAGATCCTTCCCTGCCAGATTACTGTGGTTGAATGTAAACACCACAACTCCTGCCAAG
TGGCCCCTACTCCCTCTGATTCTAATTATTATTTCCTCTTTTTGCCTCCTGCTGCTGCTAGCCTTGGGGT
GCACTCTGCTTTGTTTCTTTTCCTTAACCCTAATATACCTTTGTAAATAATCCTTTTTTGTTTTAATCTC
TCAATTAAAATCTCCTCTTGGGGTGTGTCATCTGTTTTCTGCTAAGACTTTGATTAATATAATAGCTAAC
ATTTATGAAATATTTACTATAATACAGGCACTGTTGGAATAATTCACATGGATCCAATTTTTTTATCCTC
ACAACGATCATGTGAATTAGGTACTATTATGACTAAATTTAAAATATAAGGCTACTGAAGAACATAGATT
TTAAATACCTTCATGATCATATAGCTAAAAACTAAGACAAAATAAAATCCAGGAGTCTGACTCCAGAGCA
GAGCTTAGATGTGAAAGTAATTAATATGCTTTTAAAGAAAATCTTATATTGTTGGTGTCTGTATAAATCC
CATAGACAAGATCTAAGTACGAAAAAAAAATAGAAGCAAGTCAGGAAAACTTAGTTCACAGGTAATAAAA
CTAGACACAGATATCTAAATTTAGAAATATCCCATGCATATATTCTGCATATCTGAATGCATCAAGATAT
TAATAAGCATCTTAATACTTTGGAAAAAGTATAGAGGGGACTTTCTGTCAACATTTGAAGAAAACATTTA
ACTAGTATAAATATATCATACAAGTTAACTGAAAAATCTATCTGCTGTCTATCCTCATGTTCAGTTATCA
AGTATATATCATTTAAATTTAAGATGAACAAAACACTACTTAAATCCCAAAGACTCATTAAAGTAAAACT
AGTAAATGCATTTTATTTATCAATTATTTCCCTGAAAATGCACATTCTTTTTAAATTAATTTTTTATTTA
AAAAAGAGCATCATGCCATTAAACAAAAAAAGGAAAATTTTATTTACTGGGAAATTCTTGCTGATTTTTA
CATGGGATTGTGACAAAAAAAAGGTCAGATTTTTATTTTTATTCACAGTAAGGAGAGATTTTATCTTCAG
GGATGATTACAAGGTGGGGAAAGGGCAAGGATCTCAAGAGTTAGGCAAAGATTTCTCTTTTAGAGAGAGG
AGTAAACAAGGCTAGAATGAACTGGATGGGGAGAAGAGGGATTTAAGGGTGGTATGATCTGACAGAGAAT
GTTCCACTCTGAGACCAGCCTGCTACCTTGAGGGACTATGTAAGGGGAGGGGGGGTGTGGCTGCTTCATC
TTGAAAGTAAGTCAAAATTCAGGGATTTGGGGAAAGTAGAAAATCTTAACCAAAATTTGGTTAATAGGCA
TTTTGTTCCCATTGGTCAGTGGGGACGGGCAGTTTAGCTTATCATTTATGAGGTAAAGAATGGGAATTTT
GAAGTTCTGTGTTGGGTCTCATCACAGGTGAATGGGGGACATTTGTAAGTTTTGCCTGTGCTATATGGGG
AAGGGTCATTCTTTACAGTAATTTCCTAGAACATGAAAGGATAGGGGGGTTCTTATTATTTCATCTACAT
AAACTGGAACAGAAGATGATTTCACTGGTTTATTAAACTCCTCAGGATCTTCTACCTGCCCTTCTTAACC
TTTACATTCCAAATTCACTTCTATCTCCTTTCTAGAGCTGGTAATCTTTCTTCCATGAAGTTTATGAAAT
TCTTCATGACATGCATAGACTTTGACAGTTCTGTTTCTGATGTGCATAATACCTACCAGATTGATTTGTG
ATAATATCATTGAGAGGCCACTTATAGCCTATTTATGACTCAGCAAACCATGGTCTTTTACAGCCTCTTT
TAAGATGGTTTGGAGAGATAAAGAATAATACTTCATGACATGTAAAAATTATATTAAGTTTGAAGTTTAA
TGTCCATAAATAAGTTTTATTGGGACACAGCCATGCTTATTTTTATATAAATTTTCCATGACTGTTTTCT
TCCTACAAGGTGAGAACTGTATACTTGCAACAGAGACAATATGGCCCACAAAGCCAAAATTATTTACTAA
ATCATCCCTTGTAGAAACTAAGTTGCAGACTCTGGTCTATATAGTCACATTACACTACAATGATCAAGTA
TAGATGTTTGACAGTATTTAAGTTTCTGAATGAATGAACCTATATAGAGAAATGTATAGGTATAACTATA
TTACTGATATATCCTATTAATTTTATTATTTTTCTGTTGAGTTTTTAGCTTTTCTAGGTACATAATTTTG
TCTGAAAATAATTAGGACAATTATGTCATGCCTTTCTTTGCTAATATTTCTAAGCAATCATTCCTTTACT
TATTTCTAACTAATGTTTACCCCCAGTTATATTGTCAGATTTTCCTGGAAAATATTAATAGTGCAAATTT
CCTGCTGTTTCCTAACTTTGACATAAATATTTATAGTGGCTCTTCACTAAATTACATTTACATCAATATC
CTTAGTGATATATTGATGTAAATTTGATTTGAGACAGATCAACTTCATAATGTAAGGATATTTCTCTTTA
TGAAGAGAATTTATTATAATTTATAAATAAATGTGATATCTGTTAGATGACTTTTAAGCATCTAGATGTA
GTTCATATTCTGTTTTGGCCCATTAATGTCATGTATTTCATTAATATATTTTTAAGTACTGCACCATTTT
TTCATTACTATTTTGGATCTTATTTTGGGGCATTATGTTATTAAGTAGTTGTATGTTTTATTTAAGAAGT
TTATAACTGTATTCATTAATGATATTGGCTGTAGATTTCATTTTTGAGGGTGTTCTTTCGGAAAAGTTTA
CTATCAAATTTATATTTGCTTCAAAAATATTTTTGAGTAATTTTCCTATTTTTCTATCCTTTGAATGTGT
TTACAAAATGTGGAAATAAGTTGTTTCTAGATAATATCAATATTTATTTGCTAGAAAATAAAAACTCACC
TGTAAACATGTTTTATTGTTATTTTAAAGTGGTTTTGCATATTTCTTTTAACTCATTTCCTTATTAATTT
CCTGATCAAGTTTTCACTTCTTACATCATTTTGACAATTTCACTTTCATAGACGTACACATTTTGTAGAA
AAATTCAAAGATTTTAACATAGTTAATAATTTTGTTTGCATACTTTTCATCTCTCAAAAGCTAAGAAAAA
TGAATTCATGTCTTCTACAATTAACAGCTTTGCATAATAACGTACTACTATTAACTTTATGATTTTATGA
CCAGTATATATTTATTAGAACATTTACAAGTACAAAATTACCTTACTTATCCATTTAAGTTATTTGTGTT
GAGATATGTTTTCTCTCTAATAACATTGGAAAACCTAAATTTTTGTTTGTATTTGTTGATATATTTTTGT
CCATTTTTTATTTTTAATTTTGGCAATCTGTTCTATGCTTCTTTCAAATAAACATCTGTTCAACTTTTGC
TTTGTGATCTAATTTGGTACTTTGTGTTGGTTAATTATAAATTAACCAATTCAAAATTTATTATTTTAAA
TATCAGATAATATTTGATTTTTCTTCTCATAACTTGTTTTGTGGTTTCTACTTTTCTTATATTTTTCTTG
ATTTTCCCATAGGGGTATATCAACTGCAGTTTTTCCCATCTTTATTTTTTCTTCTTCTATTTTTTCTAGC
TTTTTGGAATGCATGTATTGCCTTATTTCATTCCATTATTTTAAAAGAAACCATCAGCCTCATAAATGAA
ACTATCTATTAACTACCCTTACATAAGATAAAAATATTGTTTTTTCTTTCCCTTCCAACTATTTTCCAGT
TTAAAAAAATGTACATGATTTGGTATTCACGTTAATTTAAAAATGTTCTCCATAAGCGTATTATTTTATA
ACCTTACTAACCACGATTATTAAAATATGGCTCTATGGTTTAACTCCATATCCTAACAATTATTAAAATA
TGGCTCTATGGTTTAACTCCATATCCTAACAATTATTAAAATATGGCTCTATGGTTTAACCCCATATCCT
AACTAAGGATGTTACCATTATTACCATGTATTACCATTTCTTCTAGCATCTTCTGTAGAGCTGAAGTGTA
TCTTAGAGTCACCTCTTCATGAAGGAGATATTGATGATATATTTTCTCAGCTGCTGGTAGATGAAAATAT
TTCTGTAATGCCCTCATGTAATAATGATAATTTCAGTTGATACGCAAATATTTTTCTTAAAAATATATTT
TGTTCTTTTATCCTCAGACATCTAATATGCAGAAGATAAGATACAGCCAGATTTTTCTTCCCCGCCCACC
CCCCTTGGAAACAAACCATTTTCCCCTACCAGAAACTTGATGGATGTTTTTCTTTATCCTTTGACCTTCT
CTAGCTCAACAGGAAATGTTTTCATTTGGGGCTCTTTTCTTTTTTTTTTTTTTTTTTTTGGTGATGGAGT
CTCTCGCTCTGTCGCCCAGGCTGGAGTGCAGTGGCGCAGTCTCGGCTCACTACAACCTCCGCCTCCCGGG
TTCAAACAATTCTCCTGTTTTAGCCTCCTGAGTAGCTGGGACTACAGGCACCTGCCCCCACGGCCGACTA
ATTATGTATTTTTAGTAGAGATGGGATTTCACCTTGTTGGTCAGGCTGATCGCAAACTCCTGACCTCAGG
TGATCCACCCACCTCGGCCTCCCAAAGTGCTGGGATTACAGGGGTGAGCCACTGCACCTGGCCTATTTTC
TTTAATGTAAATACTATTCCATAATCCTGTTTGATTTACAGACTCTTTTTGTTAAAAGATTTCATTTGTT
CTTGAGATTAGAATTCGTTGCAACAACTGTGGTAGCCTTAGTTTTTCCTTTTCAATTTACTATCTTTAAT
GATAGCATCCTCAGGGCTTTTGCACCATGTATATCTTTACATTCTTAATAATTATTTCCATCTAATTAAC
TTTTTACTCTGAGATATTGGGAAGTTATCACAGACTTCCATTTAACAAACTTAATTTCTGCATTTTCAAA
TATGCTTATTACTGTTTATACTGAACTTATGAATTTAACTATTTTTACAGGTATCCACGCTATATCCAAC
TGGACTCCCTTGCTAACTGTCAGCTTTCCTTTCATAATTAAAGTGTCCTCTTTAATCTTAATCAGAGTAT
ATCCTAAAATTAAAATGTTCTCTTCTGCTTCTTATTTTGAACAATGGTATCTTTTCATATGTTTGGCGAT
TTTTTTTCCTGCTTACTCATCATTACAGAAGATACTCTATGCTGTATAACATTTGTATTTTAGATGATTC
TATCAAATATTGCTTATTTCCTGTAAACATCTAAAAGTCTTTTAGGCTGAGGAGAAGCTATAACTTTGTT
TTAACAGTTACTAGTTCAACTTGTAACAAAGTTAAAACTTTTTTTTTTAACTTTGCTCATTTAAATATAC
AGTGGCCTAAGGAAACTAAGAACCAATCACAGCAGAGTTTGTGTAGGTTTGATGGCTAGGACCAACCATG
GGAACTGACGCATCATTTCCTGAATTTAGTTGCGTTCAAGGGTAGTGGTATTCCTGTTCTTTGTCTGACA
TAACTGTCCACACCTAAAAGCTGGGCACAGACTTTCCCATCTGGTGGGAAATATTTCAGAAACATTAGTG
ACACTTGCAGTGTCCTGCTATTGCTGCAAAACTTGCTCTTCCTTCAGCCCTTGCCATGGAATTCAGGTAT
AATTGTGGTCTTCTATTGGCACATCCTTTATTTACAGACATACCTCAGAGATATTACGGGTTCAGTTCCA
CACCACCAGAAAAGCAATTATCACAAAGTGAATCACATGATTTTTTTGACTTCCTAGTCTATAAGAGACT
TATTTTTTCACTTTTTAATCATAGCCATTCTGACTGGTGTGTGAGATGGTATCTCATTGTGGTTTTTGTT
TCTGTGTTTTGTTGTTGTTGTTTTTGTTGTTGTTGTTCTTGTTGTTGTTGTTTTTGAGACGGAGTCTTGC
TCTGTCACCCAGGCTGGAGTGCAGCGGCGCGATCTTGGCTCACTGCAAGCTCCGCCTTCCGGGTTCACGC
CATTCTCCTGCCTCAGCCTCCCGAGTAGCTGGGATTACAGGCGCCCCTCACCAGGCCCAGCTAATTTCTA
TATTTTTATAGAGATGGGTTTTCACCATGCTGGCCAGGGTGGTCTCGAACTCCTGGCTTCAAGTGATCCA
CCCCCCTCGGCCTTCCAAAGTGCTGGGATAACAGGCGTGAGCCACCACACCCAGCCTCATTGTGGTTTTG
ATTTGCATTCTCTAATGATCAGTGATGTTGAGTTTTTTCTTCATACGTTTGTCCCATGTATGTCTTCTTG
ACATGTATGTCACTTGAAAAGTGTCTGCTCACGTCCTTTGCCCACATTTTAATGGTGGTTTTGTTGTTGT
TGTTGTTGTTGTTGTTTTTGTTTTTTGTTTTTTGCCTGTAAATTTGTTTAAATTACTGATAGATTCTGGA
TAGTAGAACTTTGTCAGATGCATAGTTTGCAAATATTTTCTCCCATTCTGTAGGTTGTCAGTTTACTCTG
TTGAATTTCTTTTGCTATGCAGAAATTCTTAAATTTAATTACATCCTATTTGTCAATTTTTAGTTTTGTT
GCAATCGCTTTGGCATTTTTGTCATGAAACTTTTGCCAGATCCTGTGTCTGGAATGGTATTTATGAGCTT
ACCTTCTAGGGTTTTTATAGCTTTAGGTTTTAAGTTTAAGTATTTAATCCATCTTGAGTTTATTTTTTGT
ATGGTATAAGGAAGTGGTCCAGTTTCAATCTTCTGCATATGGCTAGCCAGTTATCCCAGCATTGTTTATT
AAACAGGAAGTCCTTTGTCCGTTGCTTGCTGGAGAGATTGCAGAGAAAAGGGATTGCTTATACACTGTTG
GTGGGATTGTAAATTAGTTCAGCAATTATGGAAAACAGTGTGGTGATTCCTCAAAGAACTTAAAGAAGAA
TAACCATTCAACCCAGCAACCCCATAATTGGGTATGTGCCCAAAGAAATATAAATGGTTCTACCATAAAG
ACACATGCACACCTATGTTCACTGCAGCACTGTTCACAATAGCAAAGACATGGCATCAACCTAAATACTC
ATCAACAGTGGACTGGATAAAGAAGATGTGATACATATATACCATGGAATACTATGCAGCCATAAAAGAT
TATAAGATTATGTCCTCTGCAGGGACATGGATGGAGCTGGGGGCTATTATAGTAGGCAAACTAACTGAGG
AACATAAAACCCAATACCACATGTTCTAACTTACAAGTGGGAGCTAAACAGTGAGAACTCATGGACACAA
AGAGGGGAACAATAGGCACTGGGACCTACTTGAGGGAGGTAGGTGAGAGGAGGAAGAGGATCACAGAAAA
GTGCTTATTGGGTACTAAGCTTATTGCCTGGATGGCAAAATAATCTATATCCCTGTTACATGCAGTTTAC
CTATATAACAAACCTTCACATGTACCTCTGAACCTAAAATAAAATTTAAAAAAAGACCTATGTTTACACT
ATACTATAGTTTATTAAGTATGTAATAGAATTACATCTGAAAAAGCAATGTACATACCTTAATTTAAAAA
TATTTTCTTGCTTACAAATGCAAATAATCATCTGAGTCTTCAGCAAGTCATAATCTTTATGTGGGTGGAG
GATCTTGTCTGGATGTCAATGGCTGCTGACTGATCAAGGTGGTGGTTGCTAAAGGTTGAGTGGCTGTGGC
AACTTCTTAAAATAAGACTACAATGAAGTTTGTTGCATTGATTGACTCATATTTTCATGAAAGATTTCCC
TTTGGCATGCAATCCTGTTTGATAGCATTTTACCCACAGTGGAACTCCTTTCAAATTGGAGTCAATCTTC
TCAAAACCTGCCACTGCTTTATCAACAAGTTTATGTAATATTCTAAATCCTTTGTTATCATTTCACAATA
TTCATAGCATCTTCACTGGGAGTAGATTCCATCTCAAGAAACCACTTACTTTGATCATTCATGAGAAGCA
ATCCCTCATTCATTCAAGTTTTATCATAAGATTACAGCAATTGAGTCACATTTTCAGGCTTCACTTCTAT
TCTAGTTCTCTTGCTATTTCTACCGCATCTGCAGTTACTTCCTCCACCGAATTCTTGAACCCCTCAAAAT
CAACCATGACGACTGGAATCAACTTCTTCCAAATTCCTGTTAATGTTGATATTTTGACCTCCTCCTATGA
ATCATTTATGTTCTTAATGACATTCAGAATGGTGAATTCTTTCCAAAAGTTTTCCAACTTACTTTGCCCA
GACCCATTCACGGAATCATTATGTATGGCACCTGTAGCCCTAAAAATGGATTTTTTTTTTTTTTTTTTTT
TTTTTGAGACGGAGTCTTGATCTGTCACTCAGGCTGGAGTGCAGTGGCATGATTTCGGCTCAGCTCGCTG
CAGGCTCCATCTTCTGGGTTCCCGCCATTCTCCCACCTCAACCTCCCGAGTAGCTGGGACTACAGGCGCC
CGCCACCATGCCCGGCTAATTTTTTGTATTTTTAGTAGAGACAAGGTTTCACCGTGTTAGCCGGGATGGT
CTCTATCTCCTGACCTCATGATCCGCCTGCCTCGGCCTCCCAAAGTGCTAGGATTATAGGCGTGAGCCAC
TGCGCCCGGCCAAAATGGATTTCTTAATAAGACTTGCAGATCAAAATGACTCCTTGATCCATGGAGTGCG
GGCATGAAAACGTTAATCTCCTTGCAGATCTCCATCAGAGCTCTTGAGTGACTAGGTGCATTGTCAATGA
GCAGTAATATTTTGAAAAGAATAGTTTTTTTCTGAGCAGTAAATCTTAACTGTGAGCTTAAAATATTCAG
TAAACCATGCTGTAAACAGATGTGCTGTCATTCAGACTTTATTGTTTCATTCACAGAGCATAGGCTGAAT
ACATTTAGCATAATTCTTAAGGGCCCTAGGATTTTTGGAATGGTAAATGAGCACTGGCTTCAACTGAAAG
TCACCAGCTGCATACAGTTATCCCTCAGTATGCATGGAGGATTCGTTCCAGGACTCAGATGAATACCAAA
CTCTGTGCAGTCCCTGATATAAAATGGTGTGGTGGCCAGGCATGGTGGCTCATGCCTGTAATCTCAGCAC
TTTGGGAGGCTGAGGCGGGTGCATCGCCTGAGGTTAGGAGTTCGAGACCAGTCTGGCCGACATGAAGAAA
CCCCCCTCTCTACTAAAAATACAAAAAAATGAGTTGGGGGTGGTGGCAGGCACTTGTAATCCCAGCTACT
CGGGAGTCTGAGGCAGGAGAATCACTTGAACCCAGGAGGTGGAGGTTGCAGTGAGCTGAGATTGTGCCAC
TGCACTCCAGCCTGGGTGACAAGAGTGAAACTCCGTCTCAAAAAAAAAAAAAAAAAAAGGTATGTGTGGT
ATTTGAATATAACCTGGGCACATCCTCCCATATACTTTCAAACATCTCTACATTACTTATGACACCTAAT
ACAATGTAAATGCTATGTAAACAGTTGTCATATTGTAATGTTTAGGGAATAGTGACAAGAAAAAGTTGGC
TGTACATGTTGGGTACAGATGCAATTTTTAAAAAATATTTTTGATCCATGGTTGGTTAAACTCACAGATG
CAGAATCCACAAATATAGAGAGCCAACTATACTTCTAAATTCCATAATTCTCAATTTTATCTTTTGTAGA
AGAACTGGACTAGATTATCTCTAAAGTGTCTTCCACTTCTATATTTCTTCCAGTCCACATTAATAGGATC
ATTGTTAGGCTGGTATCTGTTTCTAAAGTACTACAGAGTAAATCAACAAGGAAAGCAAGGTTGTCTTCTC
ATTCAGAAGGCCAGAGGTTGCTAAATACTCCAGTTCTGAATTGTAAGTGAAACAGGGAAGTTATATACTA
GGTGATTGGTGTGCTGTTAAATGTTTTAACCTTGGCTTTGCTCTATTAACTTTATCTGTCCTTTTTAAAT
CACTTTCAGAAATAAAATAAGGTGAATCACCTCAAGTGGGGTACACCAATGAGTTGAGATCAATTTGGGA
CGTCCTTCATATTGACCTTTGTTGATACAAATTATCTTTGTCACAGAATATTGCTGTGATTCCATTACCT
CCTGTTGTACTTGCCTGAGCTTCCCATTGTTTTAGTCACTCTGCATCACTGTGTACGATGTCAATTGTGT
TTTAGATGGTTAAAAAAAAGGAAAAGAAACATTTCAGTCATACCAAAGTGTTGATTTGCAATAAAGAGTA
TAATTTACTGTGGCTCTGGGTATTGATTAATCTATACAATGAAAATGGCTTTTGAGTGCTTTGGTTATCA
GTTGAAATTATCTCTATTTTTTAAGCTGATAATTACTGAAAAATGTCACAAGTGGAAAAAAGGGTGCAAT
ACAAACTAGTGTTTTATCCAGATATCTATTCTGCCTGGGAAAGGTAAAAGGATACACATTATGGTTGCAA
TACGGTCTGGATGTTTCACTTCTGTGTATGTGTATTTCTTGCCCATCTCAAATAATAGGTGGGACTTTTT
TTCACTTTTATTTTAAGTGAAGGGGCACATGTGCAGAATGTGCAGGTTTGTTACACAGGTAAACATGTGT
GATGAGGGTTTGTTTTACCCATTATTTCTTTTTTAAAAAAATTTCAATAGGTTTTTGGGGAACTGGTGGT
GTTTGGTTACATGGATGAGTTCTTTAGTGGTGATTTCTGAGATTCTGGTGCACCCTGACCCAAGCAGTGT
ACACTGTACCCAATGTGTAGTCTCTTATCTCTCACCCCCTCCCACCATTTCCCCTGAGTCCTCAAAGTCC
ATTGTATCATTCTTATGCCTTTGCATCCTCATAGCTTAGCTCCCATTTGTGAGTGAGAACTTACGATGTT
TGGTTTTCCATTCCTGAGTTACTTCACTTAGAATAATGGTCTCCAATTCCATCAAAATTGCTGAGAAAGC
CATTATTTCATTCCTTTTTAAGACAAAGCAGTATTCATATATATTTGTATATGTATAATTATATATGTAT
ATACACATAGAATAATATATATATTAATCAACAAGTGATTAATGGGCGTTTGGGCTGGTGCCATATTTTT
GCAATTGTGAATTGTGCTGCTATAAACATGCGTGTGCAATTATCTTTTTCGTATAATGACTTCCTTTCCT
CTGGGTAGATACTCAGTAGTGGAATTGCTGGATCAAATGGTAATATCTACTTTTCGTTCTTAAGAACTCT
CCACATTGTTTTCAGTAATGGTTGTACTAGTTTATATTCCCACCAACAGTGTAAAAGTGTTCCCTTTTTA
CCACATCCATGCCAATATCTATTATTTTTTGATTGTATCAATTGTTGCAGGAGTAAGGTGATATCTCATT
GTGGTTTTGATTTGCATTTCCCTGATAATTAGTGATGTTGAGCATTTTTTCATATGTTTGTTGCCCATTT
ATATATCTTCTTTTGAGAATTGTCTATTCACGTCCTTAGCCCACTTTTGATGAGATTGTTTGTTTTTTTC
TGGCTGATTTGTTTGAGTTCATTGTAGACTCTGGATATTAGTCTTTTGTCAGATGTATAGATTGTGAATA
TTTTATCCCACTCTGTGGGTTGTCTGTTTACTCTGCCAAGTATTTATTTTGCTGGTCAGAAGCTTTTTAG
TTTAATAAGTCTCATCTATTTATTGTTGTTTTTGTTGCACTTGCTTTTGGGTTTTGAAGTCTTTGCCCAA
GCCAATGTCTAGAAGGGTTTTTCTGATGTTCTAGAAGTTTTATGGCTTCAGGTCTTAATTTAAGTTCTTG
ATCCCTCTTGAATTGAGTTTTGTATAAGGTGAAATATGAGGATCCAGTTTCATTCTCCTACATGTGGTTT
GCCAATTATCCCAGCACCATTTATTGAATAGGGAGTCTTTTCCCTACTTTATTTTTTTGTTTGCTTTGTC
AAAGCTGTAAACATTGGGCTTTATTTCTGAATCCTGTATTCTGTCCCATTGGTCTATATGCTGACTTTTA
TGCCAGTACCTGCTGTTTTGGTGACTATAGCCCTATAGTTTGAATTCAGGTAATGTGATGCCTCCAAATT
TGTTCTTTCTGTTTAGTCTTGCTCTGACTATGCGGGCTCTTTTTATTTTCATATGAATTTTAAGATTATT
TTTTCTAGTTCTATGAAGAATAATGGTGGTATTTTTATGGGAATTTGTTGTGTTTCCATTTGTTTGTGTT
GTCTATGATTTCTTTCAGCAGTGTTTGTAGTTTTCCTTGTAGAAGTCTTTAATCTCCTTCGTTAAGTATA
TTCCTAAGTATTCTATTTTTTTTTTTTGCAGCTATTGTAAAAGGGGTTGAGTTCTTGATTTGATTCTCAG
CTTGGCCACTGTTGGTAGTATAGCAGAGCTACTGAGTTGGGTACATTAACTTTGTATCAAAACTTTGCTG
AATTCGTTTACCAGTTGCAGGAGCTTTTTGAATAAGTGTTTAGTGTTTCCCAGGTATACAATTATGTCCT
CAGCAAACAGTGACAGTTTGACCTCCTCTTTACCAATTTAGATGTCCTTTATTCCTTTCTCTTGTCTGAT
TCATCTGGCTAAAACTTCCAATACTATGTTGAATAGAAGTGGTGAAAGTGGGCAAAATCCTGCACTAACC
AAATCCCACAGCATATCAAAAAGATAATCCACTATGATCAAGTGGGTTTTGTACCAGGGATGCAGGGATG
GTTAAACATCCACAAGTCAATAAATGTGATACACTGCATAAACAGAATTTTAAAAAATCACATGATCATC
TAAATAGATCCAGAAGAAACATTTGACAAAATCCAACATCACTTTATGATTATAACCCTCAACAAAATCA
GCATAGAAGGGACATACCTTAAGGTAATAAAAAGCCATCTATGACAGACCCACAGACGACATCATAGTGA
ATGGAGAAAAGTTGAAAGCATTCCCCCTGAGAACTGTTGTACAGATGATTTCATCACCTAGGTATTAAAC
CTAGTATCCATTCGTTATTTTTCCTGACCCTCTCCTCCTCCCACCTTGTCACACACCCTCTGATAGGCCC
CAGTGTGTGTTGGTTTCCTTTATATGTTCATGTGTTCTCATCATTTAGCTCCTACTTATGCCTGCAAACA
TGTCATATTTGGTTTTCTGTTCCTGTGTTACTTTGCTGAGGATAATGGCCTCGTGCTCCAACCATGTCCT
TGAAGAGGACATGATCTCATTCTTTTTTTTGTGGCTGCATAATATTCTGTGTTGTATATGTACCACATTT
TATTTATCCACTCTATCACTGATGGGCATTTAGGTTGATTCCATGTCTTTGCCATTGTGAGTAGTGCAGT
AATGAACATATGCATGCATGTGTCTTTATAATAGCGTGATTTATATTCCTTTGGGTATATACCTGGTAAA
GGGATTGCTGGATCAAATGGCATTTCAGTCTTTAGTTGTTTGAGGAATCACCACAATGTCTTCCACAATG
ATTGAACTAAGTTACGCTCCCACCAACAGTGTATAAGTAAGTGTTCCTTTTTCTCCACAATCTCTGCAGT
ATCTGTTATTTTTTTACCTTTTAATAATAGCTATTCTGACTGGTGTGAGATGGTATCTCTTTGTGGTTTT
GATTTGCATTTCTCTAATGATAAGTGATGTAGAGTTTTTTTATATGCTTTGTTGGCCACAACATATATAT
TGTTGGCCACATGCATTTCTTCTTTTGAGAAGTGTCTGTTCATGTCCATTGGCCACTTTTTAATGGGGTT
GTTTGTTTGTTTTTTTTTCTTGTAAATTTGTTTAAGTTCCTTATAGATGCTGGATATTAGACCTTTGTCA
GATGCATAGTTGGCAAGTATTTTCTCCTATTCTCTAGGTTATGTGTTTACTCTGTTGATCTATATATATA
TATATATATATATATATATATATATATATATATATTGCTGTGCAGAAGCTCTTTAGTTTAATTAGATCAT
AATCTTTTATCGTATCCCTTAACTCAAAAAAATATTGAACATTAAAATATTGGTAGCTTTTTTCCCACTA
TCAACCTCCATAGTAAAACTATAGTTAGGCTTTCCTTCAAATGAAAATCCATTCATTTTTTATACTGTTT
GCCTGAAACAACACAGTATTCTTTCTTAATAGTATTTTCATGTTGAGCTAAACAAACAACATTTAAATAA
GGAAAAAATATTATTATTTCTTTTTTATTATACTTTTTTTCCTTCTTCTTTCTTTCTTTCTTCTTTTTTT
TCTCTTAATATATGGGGTCTAACTCTATCACCCAGGCTGGAGTCTAGTGGTGCAATCATAGCTCATTGCA
GTCTTGAAATCCTGAGCTCAAGTGATCCTCCCTGCTCAGCCTCTGAGTAGCTGGGACCATAGGTATGTGC
CACCATGCCTGGCTCTTTATTACATTTCTGTTATAATATTTTCTCTATTATATTTCATTTTAGTTACTAA
AGAATATGATTTTGAACTTTTTTGGAAAGATCATCATTTTACTCTTTGTTGTAGAAGAAAATCTATGATG
GTCTAATGAACTACGGAAATATTAACATAATTTTAAGAATAATACATATATTTGTATTACCAAAGGCTAT
GCTTATTAGTCATTCAAACTTCAACTAATTTTTTCTTTCATGAAATACTCAGAAGTATCTATTCACCACC
ACCAACACTCCCTCCATCATTTAATTTCAAGCAGACTTGGGTTTATTCCTGGAAATGATTTTTTACTAGT
GTTCTTAATTTTAGTCTTCTTCTTTCTCCAATATCTTAAGGCAGAATAATTTTTCTTAAATGCAACTTTC
ATTCTTTCTCCATAAATTTGAGGTATCTTCACATGAAATTGAGCTTCAAAGAAGATGAAGATGAGTAAGG
GCCGGGAAAGCCAGGGAAGGTTTCTGTTTCCTGTGTATCAAGTTTCCCAAACAAGAATCTTGGAGTCCTC
TTCAGCTTCTGCAACTCCTTCTCGATCAACATGCAGTCAAACTTCAAATTGTATTGACGGAAACTCAGAA
CTGCTTAGATCATTTTCTTACTCTCATTATTTCTGCGTGGATTTAGACATTCATTGTCTTTTGCCTGGGC
TCTTGCAATAGGCTTTTATCTTATATCCTACCGTAAAAGGTTTCAGATGCTACACATCCTCCATACTACC
AAAGTAATAGGTAAAAAATAAATCTGATTGTGTCTGTACAAAAAAACCTGTTAGAACTAATAAATAAATT
CAGTAAAATTGCAGGATACAAAGTCAACATACAAAAATCAGTAGTGTTTCTAAACACTAAGAATCAACTA
TTTGCAAGAGAAGTCAAGACAACAATATCATTTTAAAGCATCAAAAATATTGAGGAATAAACTTAATGAG
GTGAAATATCTGTACACCAAAAACTATAAAACATGGATTAAAGAAATTAAATAAGACACACATAAAAAGA
AAGATATCCCGTGTTAACGAGTGGAAAGAATTAATGTTCTTAAAATACCCAGAGTACTTAAAGTGATCTG
TGGATTCAATGAAATCCGTATAAAAATTCCAATGACATAATAGATATTTTTTAAATCTTAAAATTCATAT
GGAATCACAAGTTACCCCAAACAGTCAAAGCAATCTTAAGTAAAAATAATGAAACTTGGTTGCATCACAC
TGCCTGATCTCAAAATACATTACAAAGTTGTAGTGATCAAAACAGCTTGGTACTGGCATAAAAATCAGCA
CACAAATGGAACAGAACCAAAAGCCCAGAAATAAATCCATGCACTTATGGTCAATAAATCTTTGACAAAA
GTACCAAGAACACAAAATGGGAAACGAACAGTCTCTTCAGTAAATGATGTTGGAAAAACTTGGTATCCAC
ATGCAGAAGAATGAAATTGGACCCTTATCTCACACTATATACAAAAATCAACTCAAAATGGATTAACAAC
TTACAGAACACCTAAAACTGTAAAAGTACTAGAAGAAAATATAGGGGAAAACTCTATGACATTGATCTGG
GCAATGATTTTTTGAATCTGACCCCAAAATCACAGACCAAAACAAAAGAAATAATCAACAGAATGAAAAG
ACAACCTACAGAATGGAAGAAAATATTTGCAAACCATATGTTCAATAAGGGGTTAATGTCTAAAATATGT
AAGAAACTCCAACAACTCAATAGCAAATAATAATAATAATAATAACCTGATTACAAAATGGGCAAAGGAC
CTAAATGGACATTTCTCAAAAGAAGGCATACAAATGGCCAACAAGTGTATGATAAAATTCCCAATATCAC
TAATCATCAGGAAAACACAAATTAAAACTACAGTGAAATAGCACCTCACATTCATTAAAAATGGCTACTA
TCAAAAGAACAAAAGATAAGCGTTGGTAAGGATGTGGAGAAAACAAAAGTGTTGTACACTGTTGGTGGGA
TTGTAAATTAGCCATCATAGAAAACAATATGTAAATTCCTCAAAAAAGTAAATACAGAACTATTGTATGA
TCCAGTAATTCTACTTCTGGGTATATACCTAGACATTCAAAGAAGAATTGGTACCAACCCTACTGCAACT
ATTTCACAAGATAGAGAAAGAAGGAATCCACCCTAAATCATTCTATTAAGTCAGTATCACCCAAATACCA
AAATCAGGAAAGGACACAAGAAAAAAGAAAACTGCAGACTAATGTTCCTGATGAACATAGATGCAAAAAT
TCTCAACAAAATTCTAGCTAAGCAAATCCAAGGGCATATAAAAAAGATAATACATCATGATCAAGTGGGT
TTCATACCCGGGATGCAGGGATGGTTTAACATATGTAACTCAATAAATGGGATACATCACATAAACAATT
AAAAATAAAAATCATATGATCGTCTTAATAGACACAGAAAAAATGTTTGATAAAATACAGTATCCCTTTA
TGGTAAAAAATCCTCAACAAAATTGGCATAGGAGAAACATACCTCAAAGTAATAAAAGCCATCTGTGACA
AACCCAGAACCAATATCATACTGAATGGGGAAAAGTTGAAAGCATTCCCCCTGAGAACTGGAACAAGACA
AGGTGACAAAATTAGTGTGATAAAGAGATATTTACACTGTCGTGTTTATTGCAGCATTATTTCCAATAGA
CAGGATACGGAATCAACCTAAGTCTTATCTGTAGATGAGGAACGGATAAAGAATTGCACACACACACACA
CGCACACACACACACACAATGAAATACTCTTCAGCCTTAACAAAGAAGGAAATTCTGTCACTTGTGACAA
CATGAATGAACCTGGAGGACATTAGGTTGAGTAAAATAAGCCAAGCACAGAATGACAAATATTGCATGAT
GTAACTTAGGTATGGAATCTAAAAATGTCAAACTCACAGAAACAGAAAGTAGAATGGTGGTTACCAGGGG
CTACTGGGTAGGGCTGTAGGGGTGGAGGAATGGAAAATGTTGGTCAAAGGGTACAAAAGTTCAGTTAAAC
AGGAGGAATAAGTTCTGGATATCTATTGTACTATGTAATGACTGTAGTTAATAATACTGTGTTGCATATT
CAAAAATTGCTATGTGTCGATTTTAAATGTTCTCACCACCAAAATATAAATATCTGAGGTGAAGGAGATA
TTAATTAGTTTGATTTAATCATTTCACAATGCAAATATTAAGTTGCACACTGTAAATATATACAATTTTT
AATTGTCAATTAAATATAATTGAAAAATAGAACATAATAAAATATTGGATTATTAACTCTATCAGGAAAT
GAAGCTTTGAGTAACATTTTACTGTTTATATTTATGGTTTTAGCCACCAAGCAAACATGGTGGTCAAGAA
CTTGGACTCACATTCAGGTTGCCATAGTCCAAATTCAGGCTTCCCTACTCACTAATGTGTTCTTAAGCAA
GAAATAAACTTATTTTGGCTTCCTTTGGTAAAATGGGATTAATAATTGTTCCTATCCTACATTTTATTGT
GAAGATTGAATTAATGAAATATACACGTAAAACACTTAAGACAATTCCTGGCCTTAGTAAGATTTCAACA
AAAATGGTCATTATTATGATGCTTTATTCTGTTACATTTATGGATTATTAGACAGATAAAATTGGACCTC
ATATGTGACCATTCTATTTTCCTTTATTCAAATGGAAAAAGAATGTCAATTCAACATAGGGAAATACTAT
ATATGTATAAATTATTACTTAAATTTATGTTTTTACTTAAATATATGTTTATTAAAATCAAGTGTTTAAA
TTTATATATTAACTTTTACATTTATAACATCTTATTTCTTCTGAAAACAACCAACACAACCCATTTCTTA
TCAGTGATAATTCAAATGGCCTCTTTATTCTTTCCTGAATTTTTCAATCCCAATTCTTCTCTTCTGTTCT
TTTTCAGCAGCATCTTGCACAGGGCTTTATGGAAAAGACTGAGGTGCTGTGGTGAATTTAATCTCCTCTG
TTGTTTACTTTAATCTCTTCATCCAGTTACTCTGACTTTTCTCCATGCTTAATGTTATCCCTCTTGATTT
CACAGCTTAACAATTTCTTCTTTGTTCTTGACCCCATCCTAGCCTTCTTATTTCAAAAGTCCCCTCAAGC
AAGTTTCTTTTCACATTCTTAATTTCTTATTCTTAATAACTCCTTTTTTATGCCTTCAAGCAAGTATGGC
TCTCTCTCATACTGAAAATGTGCTCTTCCGTTCTATTTTGCTACTGTTTTTATTCACTTAACAGTAAATT
TCAAACAAGTTTTGTGCACCTAATTCTTTCATTTCTTTACAACCCACCCATTGCTTATCCACTCTTACAG
CTGTACTAGAATTGTAATTTAAATATGTATCATATCAGTTTTTTTGTAACATTGATAATTTATAATATTT
AACATATTTTGTAATAGAGTTTTCTTCTTAATATTTTACTTTCCTTTTAAAAAGCCTCAGCAAGTTGCCA
TTGACTTTCGGCATTGTATTTGATACCTCTGGACTCTGGCTACTATCGCCCTTTCTGTATAAAATCTGTA
ACTTGACCAAGGCAAGAACCTCCACCCCAGGCCAACTAATTTTCTCATCTGCTATTGTCCCAACCTGCTT
TGGTTTCTCCTATAGGTCAGAGTCAGGTCCTGCTTGGGTTTGGGCCTTTGCTCATAATTTTCCCTTGCAA
TTGAAATATTCTCTCACTCCAGGCCAGCCTTTAAAGCCTAACGTAAGTACCACCACATCCACGAAGCCTC
TGCACATCAACTTCAGATCTCAATGCTGTTAACCTTTTATAAACAAAAAGCAATATAGAATAAATCCTTT
TTGGGAGCAGGAGTCAAAATGCTAGTTGCAGTCTTGCATCTGTCACTTATTTGCTTGGCCAGTGACGTAG
CCTTATTAAAAACTCAATTTCTTTATCCAAAAGATGAGGATGTTTATACCTCCCTGATCTACCTTCTAAG
ATTGTTGTACATGATAAGTAAAATACTGTGCATAAAAACATTTGTAAAAATTTTAAAAATCTGTTCAAGC
TTGTCATTGTTACCACAGATAAATCGGGTACTACCTTGCTGTCTTGGATTGCTTTTGTATGTTGTTCTCT
TATGTTGTTACTGGACTCGGTGTTATTACTTAAATTTCTCTGTATTTATTTTGACAACTTAATTAGGTTT
TGAATTCTTTGGAAGCGGAGAGTATGAATGGTATTATGAGCAAAGACCCAGAAGGAGCCACCTCCTAATT
ATTTTGGAGAGAATTATCTAAGAGGCCTAATTGGACTTGTGCTTCCCATTAAAGTATGATTCATATCAGA
GAAGCTTACAGTCTTATTGCTGGCCACATGGAGATAGTACATAGGAGATACTCGATGGGAATTCTGTCAA
CTGAAAGAGAGTCAGCAAGACAGTGTAAACATGGTTAGCGTGTCTCAGCTTACCCAGCCTGGCTTGGTGT
AACCAATATAGACTATGGAAAATAGGAAGTAGACAAAAGGTAGTGGAGATTGGGCTCATGCCATTTTCTT
TAATTGAAATACAATTTCCATAAAATAAAATACAGATATTTTTAAGTGCATATTACTTTGAGTTTTGACA
AATGTATTCCCCTGTCTGGCAACTGCTCCAATTAAGATGTAGAATATTTCTATCACCCCAGTACCCTTTT
CCTGTCAATCCCTCCTTCTCCCAGAGACAATCACTGTTCCAATTTCTATCACTATAGCTTAGTTTTACCA
GTTCAAAAACTTCACATAAGTAGAATCATAAAGCATGTTACTTTTTTTCCTGGCTTTTGTCACTTAACAG
TTTTTTTTTTTAGATTTAACCATTTTGTTGCATATGTAAGTATATCATAACTTTTTGTTGTTGAGTAGCA
CTCTCCTATATAAGTATGTTATAATAATGTTTCATTTACCTATTGATGAGCATTTGAGTTGTTCCTAAGT
TTTAACTAGTATAAAAAAGTTTACTATGAACATTCTTGCATAGGTCTTTTTGTGGGTGTATATTTTCACT
ACTCTTAAGATTGTCAGACTGGATAAAGAGCAAAGTTCAGTGGTATGCTGTTTTCAACACATATTTTAAA
TATAACACAGACAGGTTGAAAGTAAAAAAATCAGTAAAAGATACAGCATGAAAACACTAAGCAAAAGAAA
GCTGATATGGCTATATGAATATCAAAAAAGTAGATTTTAAGACAAAGGATACTAACACAGATAAAAATGG
TCATTTTATTGGGGAAAAAAAGGCCAATTTGTAACAAGGCATGACAATCTTAAAAGGGTATAACAAAGTA
GAATTTCAAAATCCAAGAAGAAAAAAATGGCAGAATAAAGTAAAAAGCAGAGAAATCCACAATAATTGTT
AGAGATTTTATCAATTTTATCCCAGTAGTTGCTAGAACAAGAAGACAAAAAATCAATGATATAGACAACT
TGAATAACATTATCAGGGATCAAACCTGCAGACCTAACTGACATTTATAGAATATTATACCCAACAACTT
CAGAATATACATTCTTTTCAAATACACATGGAACATTCCACAGGTGGTGTTATGCTAGGCCATAAAATTA
GTGACACTTCATTTCAAAGAGTATTTGACTACAATGGAATTAAATTAGAAATCAATTACAATTAGATATA
TAGAAAAGGCCCCTTTTTACAAAATTAAGCAACAGACTTCTAAATAATCAATCCATGGATCAAAGAGGGA
AATCACACACACACACACACACACACACACACACACACACACACAAATACAGTGCGCTATACGGAGTGAT
AATTAGAATACAACATGTTAGGAATCTCTGTGTTCATGCCTTTAACCATTTCATATTATTTCATAATAAT
CAAGAACTTATTAATCAAATTAGCATTTAATTATCTTCAGATGAAATGAGAAAGAGGGTGGGCAAAATGT
AGACTGAAGGCTGAGAATTATAGGAATTATGGTCACAATCAGCCAGTGTTTTAACTCTACTGCAATTGTA
ACAAAGTCATTGGATCTTGGTTCAACCAATGGGAATAGATCCTTTCAAAATGCCAATAAATAAAATGCTA
AATAAACATTTTGGCAATTTTCTCTTGATAAATCTCTTATAGCAATGTGCATAATGCTTCCATTTTCATC
CAATGAAGTACTAGTGAAAATAGTCTAGATAGTGTTAATATTGATAGAAATTATAACTAAGGAATAAGTT
AGATGGTGATTAAAGAGTTGAATAAGCACTCAAAGAAAGTTGTGAAATCTCTGTTCTGAGATATGTGACA
AAACCATCTGTCTCTGATTTTAGTTTAATCCTTAATGAATATAAGAAGCCTGACTAAATGATCTCTTGGG
AGGCCCCCTTCCAAACACAAAACCCATTATTTTAATTTGCATTGGTTATAGTGCCAACACAAAGAAAAAA
CACACTCAAGATCTTTTTTATAAAGTAGGGATTTAATTCTAAATCTTAGTCATATCTTACAATTTATAAA
TTCTTTAATGTTGAAAAATATTTTCTTTGTAGAACTAAAACCATTCTCTCTCTCTCCCTCCCTCCCTCTC
TCTCTTTCTCTCTGTCTGTTTGAGAACCCAAAATAAAATGTAGTTTATAATTTGTGATTATGATTTGAAC
TTTTCAAAAAATGTATGAGTCATTTCTTGCCCTTTGATAATTTCCAAATGATGATGCTTTGCTTTTGCTT
TTAGAGATCAGTCTTTGCTTTTAATTATTTCCATATATTTTGGTTGGGAGTGAGAAAAATAAACGGAAAA
TGTCACTCATGAGTGTCAGCCTGGTAATGAATATATAGAATGCAAATGGTCACTTTACTGGGGGAGACGG
TAAGTTTTTCTGAGCCTTTTGCCCTTTTATCTTCTCCAGCCTGCTGTTTTTTCTGTTTTCATTTGTAGAT
TAAACTGGTGCAGCAATATGGTGGGGTAGAAATCACATGAATCTGACATATGTGGGCTCCTGTATCTGTG
CACATGGATAGAGATGCTGGTCAAAATCCAACCATCTTATGAGTTCCAGACATACTTTTAAAATTCACAT
GCATGAAGCTATACAAAATATTTTAAAATACAGAATCCAAAATAAAATTTCTTTTTGTATAAAATACTCC
TTCTCAATTCATTCCTGTAGGCAAAACTTGATATTTAATAATATTAAGAAAAAGAAGAAAATGAATTCGA
AATATTTTATTGCTTTACAACACAAAAAAAATGGTTTACTTACTCCCAATAATCTGAATTTATTCCAATA
GTATATTTATGGATTTAACCAGGAGAGGGATTATCCTTATTCTAAGTAGGGGAGAAAAAATTAAAAAAAA
AAAAAAAGAAAGGAAGGAAAAACTCCAGTGGTCAAGAGATTGAGTGAACCGAGGAACAGAGTGGTTATTT
GTTCTCTGTAATGTATTAGGAATGACTGGAAGTAATGGAATATGCTAGTGAATATAGCTACCCCTATATT
GAATAGAAAAATACATTACCAAAACACTTTTCACACACGAAAACAAAAGAACATTTTGTACAAATACCTG
ATGTGTAGCTATATCATTTAGTTGCTACTATTGCCTAGTTTAAAAATTGTACCCTGGTTAAAAACAGAAT
CCCTTTTCTAATCTTTTAAGATTTCAACAAATATTTCCAGGCATTAAGGACAGGCAGTATTTTGCCTTGT
CCACTTCTCTGACGTCTAGCTGTTTCTAGTTGCTAAATTTGGCTGCTGTACTTTTCATATCCGTAAAGCT
GTTCTTGGGTCCACCCTGCAGGCAGCCATCCAGGTTTGGATTTTTCCGGGCAGCCCGGGCTTGGCTATCC
TGCTTTCATCATCCTCCTTCTGTTCTCAAGCCAAGGACATAGAATGGCAGAGAGCTCCCCTTTATTTCTG
GTATGATTGTTGCTTCTCCGGGTCTGGCTTGGGCTCTGATCTTGACATTTATGTCACAGGTGTCACTGGT
GAAACTGCTGTGAAACCAGATGGCAGGCCTAACTGAGGTTGGCAGCAACTGCTGTTGTCCCCAGTGTCAC
TGCTACCTGCTCTACTTTCTTATTCCTGTTGATGTTCTACTGCCTTGTTTTATTGCATTCGTTCAATGTT
ACCTTCCAACAGGCCTTTTCAAATGTGAACACCATTCCATAACTCTTCTGATGCATTAATTCCCCATGGA
ACCCATTCATCTAGGGAGGCTCATGACTTGGCTAGATTCTCCACTTTCCTTCAGGGCTTTTGTTAAACCT
CATTTCTTCAGCAAGCCTCTCCATGACCACCCTATCTAAAATTGCAATACATCCTTCTCCTCCCCAACCC
TGATTATTTTTTCTCTCTTTTTCATAAATTTTCTTTCCCTGGAGCAATTTGTACCACATGTCAGAATCTA
TATTTGCCTTACTTGGGCATTATCTGTATCCCTCCACTGGGTTACCTAGAATATAAGCTCCACAAGGGCA
AGGTTTTGTCTGTTTGGTTCATTACTGTATCCCAAGCATCTGGAAGAAGACTGGCCCTTAGTAGACATTT
CAAAATTTTTGTTAAATGATTAGCTGGATTGTAAATACTGAAAGATTAGGTTTATTGTTTTTGATTTGGT
TTCATTCATTGTGCTTAGTACAGTGCTATATGTAAGAGCTTGAAAGACAGAGAAAAGAGCATTAGTAGTA
TCTGTTATGTATACTGGATGTTTTACTTGAGTTATACCCTTTTTTTTTTCCAAGACAGAGTCTTGCTCTG
TTGCCCAGGCTGGAGTGCAGTGGTGTGATCTCGGCTCACTGCAACCTCCGCCTCCTGGGTTGAAGCAATT
CTCCTGCGTCAGCCTCCCAAGTAGCTGGGATTACAGGCACCCGCCACCACGCCTGGCTAATTTTTATATT
TTTAGTAGAGACAGGTTTTCACCATGTTGGCCAGGCTGGTCTTGAACTCCTAACCTCATAATCTGCCCTC
CTCGGCCTCCCAAAGTGCTGGGATGGCAGGCATGAGCCACTGCACTCAGCCAAGTTATACTCTTTAATCC
TCACAATAATCTAGGTATTTTGACCAAAGAACCACAGCTAGAAAGTAGGAGAGTAAGATGTAAACCAGTT
TTGTCTGGCTCTAAAATTACTGCCTTTTTTTGTTTGTTTGTTTTGCTCAGAAGAATTGGTCATGCCAAAT
AGTAATAAGAGTTCTTCAAAATGAATGATATTTCATAATAAGGTGTAGCTAGAAACAGTTTGCCAAAAAT
ACTTAGGAAAAAAAAAAGATTTGTGAATTAGCTCCCTAAAAATACAAACGCAACAATTCCAAAAGAAGGA
ATTCCAAAGTTATTGGGCCATAGTACTTTTGTGTTAAGTGCCTGGCCCTCCAAGGTAACTAATTTTAATT
TAAAAAAAATGTCTGGACACATATTTTGACATGTTTTGCTGAAAATATATTTATAATATGAGCCCAACAA
AGACAAATAGATTCATTCTTGGTGCTAAAAAATGACAAGAAAACTTCTACGCCATTGCTCTCGAATGCTT
TCTTCATTGTAGTCCAAGAAAATTTGATTAGTTGTATTTTAAAAGTAAGCCAGATTTCACTGGTAAGGTC
TTTCCTTACTTCATCTTCTCCCTTGACACCTTTTGATTTGGTTGTACAAATGCAGGTTAGGCCCCATTAT
TAATTCAGTACCACATATTTATGAAGACCCCACATGATGCAATCAGCACTAGAGACTGGAAGTTTTTCAT
CAAGCCCAGTAAGCCCACAGTAAAAAGGAATCAGTCTCAATCACTCATCAGGAAGCTAACATGTTCTGTG
ACCACCTTCTGGATGTTGAGTTATCTCTAATCCAGAGTTTATGTCAATGATCTTAAAAATTAGTTCAATT
CTGGATGGGCGCGGTGGCTCATGCCTATAATCCCAGTACTTTGGAAGGCTGAGGTGGGCGGATCACGAGG
TCAGGAGATGGAGACCATCCTGGCTAACATGGTGAAACCCCATCTCTACTAAAAATACAAAAAATTAGAC
AGGTGTCGACAGAGCAAGACTCCGTCTCAAAAAAAAAATTAGTTTAATTTTGTCTTCATTTTTTCTTACT
TAGTAAAAGATAATGTAATGCCTGGTGTTTTTTTAAGGCAATCATAGAATCTGGAGGTTGAAAATATTTG
GGGAATTATACATTCTAATTATTCAGACAGGCATCCTTACTGCAGTATCTGCTAGTAATTGAGGATTATT
TCATATTTAAAAATACTCATATTTTTATGCAAATCTCAGAATTTGAACATTGTTACTTATAATTGTTAAA
AAGTAAATTGAGAAACAATAAAATGTTAAAGAGCTTATTTGAGCAAATGGTAATTCTTGAATCAGCTCCA
AACCAAAAGTGGTTCTGAGGCCACTTGAGAGAACTCAAAGAGAAAGCTTTTATAGGGAGAATGTAGAAAT
AAAGCAAAGGAAATATTTAATTGGTTGCAATTATATAGTTGCCTTATTTGGTCTATCTTGCTGAAAAGTT
CCTACTTGTATTACTACATGAGTTGGCAGCTTCTGATTGGTTAGCCTTAAGTTTCATTTCTCTTTAGGCA
TTTATAAGAAATAGTTCAAGTTGAGTTTTGCATATGTTTGCAAATCAAGCAAAGTTTAGGTTACTTCTGA
GGGCCACTTGGATTTGTCTGCTCAGAACTTCTTCAAGCCTGGTCTGTTTTAATTTACTTCAACATATTTT
ATCAACATTTTGCTTTCTATAACATTCATAGTAACCTTAGATCTGCCCTCTAGAATCGACAGAGGCTATT
TCACCCCCAGCCTCTTTTCAACATGAGAGTTTGCTCAATGCTGCTTCTATTTGAAATCTTCTGTTATTCA
GCTCTTTTAATGAATATCTACTTTCTGCAAGGCATTGCTTTATGTACTGCAAGAGTTACAAAGAGGAGTA
CATCATAATCTTACTTGAAGGCAATTACTTAAATAGAAAAAATGGTGTGTGAAAAACAAATGTTCTTAAG
CTTGAAAAAGTGAAAGTGTGTACCCAGCTGGAAGGATGTGAAAGTGTGCATGGAAAATGTATTTGAGATT
TAAACTGCTGTTGTCTTCTGGGTTGAATTGGTGTCATGAACTTCAGGTAGTGAAAATAGCTTAAACAATA
CCCCTGAAGTGGAAACACAGAGATTAGTGTGAAAATTTAGAAGACCGTGAGCACATTATATTTTGTGTGT
ACCATAAGACACTGGTAGAGTATATTAGTCCATTTTCTTGCTGCCATAAAGAACTGCCTGAGACTGGAAA
ATTTACAAAGGAAAGAGGTTTAATTGATTCTAAGTTTGGCATGACTGGGGAGGCGTCAGGAAACTTACTA
TCATGGCAAAAGGCAAAGAGGAGGCAAGGCACCTTTTTCTCCAGGCGGCAGGAAGAAGTGCTGAGCAAAG
AGGGGAGAGCCCCTTATAAAATCATCAGATTTTGTAAGAACTCACTCATTATCACAAGAACAGCACTGGG
GAAACCACCCCCATGGTTCAATTACCTCCACCTAGTCTCTCCCTTGACACTCGGGGATTATGGAGATTAC
CATTTAAGATGAGATTTGGGTGGGGACACAAAGCCTAATCCTATCGTGGGGGTTGGTAAGAGGGAAGACT
AGAAAGGAAAGCTGGACTAGGCAAAGTGGCTCATGGCCATAATCCCAGCAGTTTGTGAGGCCAAGGCAGG
GGGATTACTTGAGCCAGGAGACCAGCGCTGGCAACATAGTAAAACCCCATCTCTACAAAATGTTAGAAAA
TTAGACAAGTATGGTGGTGTGCACTATAGTCCCAGCTACTTAGGAGGCTGAGATGGGAGGATCTCTTGGG
CCTGGGAGGTCAAGGTTGCAGTGAGTCTTTATCACAACACTGCACTCCAGCATAGGCAACAGAGCAGAAC
CTTGTGAAAAAGAGAGAACGAGAAAAGAAAAAGAAAGAAGTCAAGAAAGAAAGGCAGGCAGGCAGGAAGG
AAGGAAGGAATGAAGGAAGGAAGCAGGGAAGGAAGCATTCTTTACATGCCAGGAAGAGGAATCTTAGTGA
TGAGAAAACTGAGTCTCTGAGAGTTTGAGCCTCACTCAAGGTCACTGAGTTGCTAAATGGCAGAACTGGA
TTTCAATCCCAATAATTTGATTCAAAACCCATACTCTTTCCCATATACCACATTATCTAGGAGGTGTCCC
AAGAGTTCTGAAGGGGGAGTTAGCTTCCATCTCAGGAGACGAAGAGAGTCTTCTTAGACCAAATGGTATT
TGAGATAATTTTGATAAGTTTATGTTGAATTCATTGGTTGAGATAGGGAAGACAATTCATTTCAAATGGA
AGAAACTGAGCAAAGGTACAAAGGGGAGGAAGTGGAGAGATTGGGAACAGAAAATAATTAATCTTTGGTT
GAACATCATCAAAAAGGTATAAAGGATGGAAATGAAAGCTAAGATCAGAGCACAAAAGGATAACATATTT
GAACTTTTACTTGGACTTAAATGATAAAGTTTTTAGACAGATTATTATTATAACTACAGCCAAGTTTTTA
AAAATATTATATTAGTAATTATATATAAAATAGATGGATGGGAAACAAACGGAAGAATGGAGATTATCTG
TTTAGAGTAACCCAGTTAATTATAATAAAATATTGAATCTGCATGTTGGCAATGGAATGAGAAGGGAAGA
GATAGATATGTAATTTCAGAAGCACAATTCACAGAACTTGATAGAGATTAGTGGAAGGAGGAGGGAGATA
GATGACATTAATTCTGTGATATGGTTTGCCTGTGTCCCACCCAAATCTCATTTTGAATTCCCACGTGTTG
TGAGAGGGACCCAGTGGGAGGTAACTGAATCATGTGGGCAGGTGTCTCCCGTGCTGTTCTTGTGATAGTG
AATAAGTCTCATAAGATCCGATGGTTGTATAAGGGGGAGTTTCCGTGCACGAGCCCTTTTTCTTTGCCTG
CCACCATTCACGAAATATGTGACTTGCTCCTTCTTGCCTTCTGCCATGATTGTGAGGCCTCCCCAACCAA
CAGGTGGAACTGTAAGTCCACTAAACCTCTTTCTTTTTTAAATTGCCCAGTCTCAGGTATGTCTTTATCA
GCAGCGTGAAAACAGACTAATACATTCTGTAAATTTGAATGCAGGAGATAGAACCTTTAAAAGCAATGGT
AATTTGTCTTGCAAGGCCTATGTTTATCTCTACTATGTACTCAGACTATTGTACAGCCTGGACCATAGAA
GGTATGCAATACGATTATTTATCTATGTACTCAATCCCTACTCTGTGCCAGGGATTGTCCTAAGCACTAG
CGATAGAGTAAAAAGAAAAGAAAAAACTCTTGTTCTCATTAGTAAAATAAACATCACGTTGCTTCACCCA
AACTAGATCACCTTAGTCCCTTTTATTGTTATCTCTGTGTCACTCTCCTGGCAGGTGAGTGTTTCTGCTT
TTAAGAATCTGTACCTGTGTCTTGCTTTAGATAACTGTCTTCAGATTATCTGAGTCATTTTTTCCACTTC
AGCACCAAGTCTGAAGTGCCGGTGAGTTTACGTGCCCCCACCCATATCCTTAGCGAAGACTGACTATTGC
AGTCTTGTGAAAGTACAGATCCCTTGCTTCATATAGGGACAAACTCAAATACATAATTTGTACTCCAAAG
TTGTCCTGTGGGATCATAATAAAGCCACTTCTGTAGGGCTTTGTTTGGAATTACACCTTAGCTTGGCTCC
TTCCCCATCCAGACCTGCAGCTTTACCCACTCCTTTCCTGATTGATCCTAGGAGTACCTCCTTAACTAAT
CACTTTCCCATTAAGCCACATCTCAGTGTCTGTCTCTGGGGAACCTGACCTAAGACATGTAGTTTACATT
GCAATGTACAGAGACAAATAATAAACAATATAAATAATTATTAATAAGTTTCATACTAGGCCGGGCATGT
TGGCTCATGCCTTTAATCCCAGCCAGTTGGGAGGCCAAGGCAGGAGAATTGCTTAAAGCCAGGAGTTCAA
GACCAGCCTGGCCAACATAGCAAGACCCTCATCTCTACAAATAAAATAAATTAGCCAGGCAAGGTGGCAT
GTGCCTCTAGTTCTGGCTACTTGGAATGCTGAGGTGGGAGGATCACTGGAGCCCAGGAGGTCGAGGCTGC
AGTGAGCTTTGCCACTACACACTAGTCTGGGCAACAGAGCAAGACCCTATCTCTAAAAAAATAGATATTT
TATTAGGGGAAGTTGAAGTTATAGAGGGAAATAAGGAAGGAGATGTGGTTAGAGTGTGTGAAAGTGTGTG
TGTGTGTTTTCAGTTGTAGGGGATGGTGTTCAATTTTATATAGCATGGTTGGGGAAGGCATCACTGGGAA
GGTCCTATTTGAGCAAAGACTAGTAGGTGCTAAGGGAATGAGCCATGCAAATTTCTGGGAGGAAAGCATT
TCAAGCAGAGATGAAAGCAAGTGCAAAGCCATGAGCTGGGAGTGTCTGGAGTGCTTGGTGAGAAGTAGCC
ATGTGAGAAAGTACAGGGCAGGAGACGACATGAGAGAAGGAAGTGAGGGGGGTGGGCAGACCTTGCAGGC
CAACACAAGCCATGGCTCATACTTTGACTTTTCCTCTGAGTAAATAGAGAGTCACTGTAGAGTTCTGAGC
AGAATAGTGACTCGACTGTTTTTGCGTTTCAAAGGAATCTGGCTACCACATTGAGAATAAACTGAAGGAG
GCCAAAAATTAAGCATGGAGACCAATTTAGACATTATTACCAGGGCTGGCTTCGTGGGCAGCCACACAAG
GCTCCATGCTAAGAAGGGCCCCACACATGGAGCCTAATGCTCTGCAGTCACCATCTTGATAATTTTAATA
ATTTGTGTTTGAATTTAGGTTTTGTAAGTGAAAGTCATGGGACTGTGGAGCATGTTCTGGGTGCTCAGTG
TCTGGTACTTCCTGCTGTGGATAGGTTCAACACTACCTGCACCCCTGCTCCCTGCCCAGGCCACTCAGTG
CCCCCTTTTTCACCTTTTTCCAGCAACTATTGCCACCCTCCAACAGGGAGAATGCTGGGCTGCTGGTGGG
AAGATGTCTGTGTTCTGCTGCCCTTAAGCCTGTCGGGGATTTGATCACAAGCTCAGGTCAAGTTGCAGTT
AGGAATGCACCTCAGGCTGGAAGTGCCAAGGCAGGTGAGAGACTCAGTGGGAGAGAGCTTCTCACCCACC
TCTAATCCAGGCATGTCAGGTGCAGAGTTCCAATGCTTTGGGGGGTTGACCTTCCACCAGTTTGGGTGGC
AGGCTCATGGGAAGGGTAGATTGACTTCTCTGACCCCAATTGAGGGCAGGTTGCTTCAGTCCAGCAACTG
GTGGGAAGGGGAATTTGGCAGTGGGGGATGGGGGCAGACATTATCTTCTGCTCTGAATCAGGGACACTCA
TATGGCTCTGTGTTTGCCCTGCAAGTGTTTCCCTGCTCCAATGAGTACAACATTAAATAGCAAATAAAAG
CCATCAGATCAGTAGACAGTTTGCATAAATAAGAAAAAAGCTATGTTGCATAATCTTTTAATGGCCCCTT
TTTCCTGCTACTTAAACAAGTGTCTTCCTCTTTTTTTTAGAGACAGGTCTTGCTATGTTGCTCAGGCCAG
TCTCCAACTCCTAGCCTCAAGGAATCTTCCAGTTTGAGCCTCTGGAGTAGCTAGGATTATAGGTGCAAGG
CACTACTCGCAGCTGGGGCCTCAACATTATCAGTTTTGCACTGTACTTCATAAATTACTGGCCCTAGCAA
TTGCAGTAATCTAAGTGAATGATGGTGGCAACTTGAACCAGTGTGCTAATGGTAGAGGAGGTGAGAAATG
ATTGGATCCAAGATATATTTTGAATATAGAACAAAAAGGTCACTAGAGGTTATGACATAGTGTATGAGAG
AGAGAGGAAGGAATCAAGGATGGCTGTAAGGTTTTTCACCTGAGGAACTTGACATTATTTGTTGAATACA
TAAATGATGTGTCCTGTTATGTATCTGTTCATGAAACACATTGATGGTAATGAAGATGCAGTGGCAGAAG
CAAATCATAGAAGTATTTCCTAGCGGTAGAAATATTAAGACTTGGTGACAAAGATCATAAATTAAAGGAA
AGGGAGTGAAACAACTAAGTCAGGAAAAATACTTCTAACTTAGGAAAAAAGAAGAAAAAGGAAGGAAATG
GAAATATTTAACAAATGGTATAAATTTCTAAGGAAGATAAGTTCAATTTTGGAAATGTTCACCTGAGGCT
CCAAGAACCTACAGCCCAGGAGACCTTAGCTATAGGTTGTTGGGAATATGTGATAGGATCCTGAATGAGC
ACTCAGGACTAGGGATCTATGTTGAGGGCATCATATAAGTGTAAGCTGGAGTCACAAGAATGGTTAACTC
ATCAATAAATGAATATGAGGAGAGATGTATACAGTATTGCAGAAGAGAGTTCCTTGTAAATGTGTAAGCT
CATCCTCCATGCCTGGGATAAGAGTACATTCATTATGGTGCATGTGAAGGTATTGAGAAAACAACTTCCG
CTTTCTGGCAGAGGTTAAGCTAGTCCCATAATAGCAATAGCAAGATTATAAGGGGAAACTATGTTATTTG
TACTGGGTCACAAATTTGTCTAATAGCAGTGGACCTGTGGGATTCTATAAACATCTTAGGTATAGAAAGG
GATCCAGTGACTTTTAGAAAATCAGACTTATATCATATTTCCCTTCTTAACTTGTAGCTCAGTGGTGCAT
TTCTTACCCTGGGGGAGAAATGGAACATGGACTATATTTAGTATTTGAAATCAGACACATGTGAATTTAA
ACTCTGGCTCAGAGTGGCAATGGCAAATTATTCAACATCCATACACCCTGATCACTCCCACATTTGAAAA
GTGACAACAATACTACTTCCTTCACAAATAATGCAAGGATCAATGAGATAACAAATCAAGAAGCCAGTAG
TGTGCCACAATTTAGGTAGTGTTGACATTACATGAAGAAATGAACCCATGGTCTCCTATGCTCTCCTTCA
CTCCTCTCTAGTTCTCTCTCTCTCTCTTTCTCTCTCTCTCTCTGTCTGTTTTTCTCTCTCTCTCATACAC
ACAGGCATGCATACATACACTTTGGAATGTAGCTATTTTTTGAGCATTGTTTTATTATTATTTAATTTAT
TTATTTATTTATTTTTTGAGACAGAGTCTGGCTCTGTCGCCCAGGTTGGAGTGCAGTGGCGCGATCTCGG
CTCACTGCAAGCTCCGCCTCCTGGGTTCATGCCATTCTCCTGCCTCAGCCTCCCTAGTAGCTGGGACTAC
AGGTGCCCGCCACCACACCTGGCTAATTGTTTGTATTTTTAGTAGGGATGGGGTTTCACCATGTTAGCAG
GATGGTCTCAATCTCCTGACCTCGTGATCTGCCCACCTCGGCCTCCCAAAGTGCTGGGTTTACAGGCGTG
AGCCACTGCGCCCAGCCTGTTATTATTATTATTTCAAAGACCTTCTGATCCTGGCTCTACCTGCTCATTC
ATGGGGAAGATGCCCAGACATTCAGATCTACTGTTCCACTGAGGAGTTTAGAGGCTGCTTTTTCTCCCCC
ATAAAAGAGAATGGGGACTGATGGAAAAATTTTAGGTAGAAGTCTTTGATGAAAAGAAGAAATGAGGAAA
ACTGAGCTGGAAATATTCAGGTATATCAAACAAGTGGAAATACCTCACCCTCATAAAGAGAACCATATCC
TGGGAAATTTTCTTTCTTACCTGATTATTAAAATCCAGAATTGGAATTCTGTGATTCCAGTATAATCCAT
CCATCAAAACCAAACTACTATGTAATCAGATTAGAGGTGGCCTTCCTGCTGATGAGCCATTTAGAGACGG
TTGAATGAGTTCTTGGAAGTCTGCTAACATGCACTTAATACATCTGAAGATTTTAAAACGTTGCAGCAAT
GTATTGCCAGTCTACCTACAGTAAAATAAAAATGCTTCCATTAAAATACAGGTATATTTCTTTTATATAG
TTAACTTATTTTTCCTAGGAAAGTTAGATGTAAAGTGATTATCTATACACTGAATCATATTTCCCAGAGA
TCATCATAGTAAGATTGGTATGCATTTCTGTTAAAGAAAAGCAAAATGTGTCTTAGGACATAACAATCAT
TTAATGGAATATCTAAAGAATGTTGGTCAAGCATTTTGAACAAGATGAAGATGGAACCAGGGCTGGGTGT
GGGCAGTAGACTGTTCCACCAGGAGCCAAAGATGACAGTGAATATACACTTGACATCAGTCACCAATGAC
AACCTAATTCTACGTGGCATACTGGCCTGGATCAATGAGTCTCCTGAAGTTGAATTTGACAAAGATGGAA
CAATTGTGCTCAGGGGCTGCTTATTGTTGCTTTATGGTCATCCTCTTCCCTGGCTCCATTGCCTTGAAGA
AGGTGAAATTCCATGAATACATTCATAACTTCAAAATACTACAAGCAGTATTTTTAAGAGGGTGGGTGAT
GACAAAGTAATTTCTGTGGACAATTAGGAAAAGGAAACTTTCAGGACAATTTTGAATTCATTCAGTACTT
TAAGAAGTTTTTTGATGCAAACTGCAATGAAAAAGACTATAACCCTGTGGCTGCCGGACAAGGTCAAGAA
ACTGAAGTGGCTCCCTCTATTGTTGCTCCAGTTCTGAATAAACCGAAGTAACCTCTCAGCTCTAGCAATC
CAGCTCCACATAGGACCATTTCCACAAATAACTGCAGCTTCTAAGGCTGCAGCTTCTAAGGCCCCAGGGT
GGTATTAAAAAATCCTGGTGTGGGCAGTAGGAGTGCTGCAGGGTCAAATTGATGCTAGCAGGTCAGTGTA
TTGAAACTTGGCATTGAAGCCTTGGAGAAAGAGATTTCTACTTCAGAAAGCTAAGGAACAATGAATTGAT
TTGCCAGCAGAACGAGGGGCAAAATGACCCTGTATTGCAGAGGACTGTAGACATGCTCTGTGCCACAGAT
GAAGACTTCGTGATATCCGATGAAAGGGGCCCACAGGAGGAACAAGAAGAGTTTTAACAACCCGAACCAG
GAGAGCCTTATCCGAATTCTGCACTCCAAATCTTGTGCTTAACTGTTAAATACTCCCTTTTATTATTCTT
AGAAGACTCACTGGTTTCTTTTTATGAGCATGAAGTACCTCTTTTTAAAGTGCATTTTGCAGAGTTTCAC
TCCTTTTTTGGTGAGTTTGAGTTGGGAGTTTTACTGTGCAGCAGAGCAACATTAACATCTGGTTAGTTCA
CCTGGGAAACAAAGAGGCTGAACATGGGGCTCACCATGTGCATGTAGGTAACATTGATTGCTGGAGAAGG
TGTTTGGTAATATGCCGAAGTGGAGACCTTGGTACAGAAATGTGAAGACTGAATTGAATTTTAACCTAAT
GTGAAATCTTGGTAGAGAATTTTGTAATAAGTTAATGCCTAAAGAATATTTAAAATATGCTTCCATATTC
AAAATATCAAATGTAACACATCAGAACATCTTATGTGTTTGACATTGTATGTTGGAAGGAAGGGCCAGAC
ATCAGAACCTTTGGAACCTGGTGTCATCACAGGCCTTACAGGGCTGCTTGCAACCTCACAGGCCTAGACT
TTGGCCCCAAAGGAAAGTTCTAAATGTTGCTCTGTAAATCCATTTGGTGTCATTGACCAACTGCATCCAG
GCTAAAAAGCAAGAGGTATTGTTGCCTGGATGAACACAGGGTGTGTTTCAGCCCTGAGATTTTTGGGTTG
AAGAGCTCTATTTTCATTGAGGATTTCTCTGGAAATTTTTCCAATTATCTCTGAAATTTCTATGTATTAC
GCTTTTTTGGGAAATGAGGTGTGTCCAATTCTTTCATCTAACAACGCTTTTGGGGATTTGCTCACATCTC
TGAGATTTGAATGGAGGTTGTTTCCCATTTTACCATCCTTTAGTTTTACATTTAACATGTCACCAGTGGT
GAATCCATAGAGGTCTGCAGCAAACTTGATCCTTGCCTCGTTGGAGGAAAGAATTTGGCTGAGGGGAAGA
AGTAGGTTTAAGGTAGAGGGAGAGACCGAGGCAAGTTTTAGAGTAGGAGTAAGAGTTTATTAAAAAGTTT
TAGAGCAGGAACAAAAGGAAGTAAAGTATACTTGGGAAAAGGCCAAGCAGGCAACTTGAGAGGTCCAAGT
GCCCTGTTTGGCCCTTGACTTGAAGTTTTATACATTGGCCTAGTTATGGCCTGGTTACGGCGTTTGCATC
TCTCTTCCCTTTATTTTCTTTTGGAGCAGGCTGTACACATGTGCAGTGATCTGCCAGCACTTGGGAGGGG
CAATATGTGCAGTGTGTTTACTGAAGTTGTGTGCATGCTCACTTGAGGCAATTTTCCTTTACCAGTAGAG
TGTTCCTGAGAAGGTCATATGCCAGTTAAACTCTGCCATTTTGCCTCTTAATGTGCATGCTTGAGCTCAC
TCACCCAATTCCTGAGAACTTATCAGGAAGCCTCTGATCACCAGGTTCAGGTGTTTTTTTTTTTTAAGCC
ATTAGGGGACTGTCTTTACCTGGTGCTGGCTAAGACCAATTATTATTTTAGACAGTTTAGTAACCACCTG
ACCGTCATCTGATGGTTGCCTGACATTCCTGAGGGAGGGGCTCTCTACTGCCTTGCTCATGTCTGCCTAA
CTACCTACTCTAACATTGCTTCTCTTCTCTGCTCCCCTTGCCCACTGGGGACACCTCTTTGGGTTCTTAA
AGTTTGCAGCTTGGAGTTGGAAGTGCAGCAGGCAGGTGGGCACACTGCAAATTCTTTGTGGACCTCTGGC
AAAGGGAATAATCAGGGAAGGCTCTGGTTACCTCTGCAAAGCTGGGATGTTTTTGGTATCTGCTGTCCAC
AGCTCTCCAGTTCTCTGAATACTTTATCAGTACACTAATCTCTTAAGAGATAAAATTTATTAGTGTGTTG
CTAAATGTTCATTTTCTTTTACAGAAAATACAGTACCATGTCTGAATTATTCATATTTAAAATTTGTTAT
TCCTTAACTCTCCCTCATTTGATTTGTGCACAACCTATTCCATCCTTTTGTTTGGCAGAAGTTTGCAAAA
TATGTGTCACTCACTGAGATTGTTCAGCCCCTGATGCATTTGTATTGATTTGTGTCTGGTGATAGCTTGT
CCTAAAATGTGTGTAAAAAGTAAACATTTTATAACAAAATTGTTGTTTAATGGATGCTTTGTGTGGAATT
CAGAGAAAAATCCAGATTCAGTGATTAACAATGCCAAAAAATGCAAGTAAATAGCCATTGTTCAAATGAC
AGTGGTGCTGTTTCTCTTTTGTGGTTTTTTAGACTTTTGTTACCTAAAATTCTGCTTTATTAGCAACTCA
TTTTTCACCTGATGTTTCTTGACAGGCTTTTTTTTTCTATTTTAAGTAGTTTCTAAATAATTTTTTTTTA
TTTCAAGAAAGAGAAAAAAGAATATTGCTCAATGCACCTAATATAAAAATAAACTATAAAAAATAAGTTA
GTTTCATATTGGAACTAAACCTAGCATTAAAAAAAAAAAACAGCTTTTCTCACCAGTAAACACTGAACGT
GAGTTCTTCATATTCTACTATTCTCTGTTTCTACTCTACAGCAGAAAAACACGTAAGTCTGGTTGTTCAG
AACTGTCTCCCCATTATGGAAATTGTTAGTTACAGAAATGGGCTAGTGGATTCAGGTTAAAAAGAGAAGG
ATACATATACATGTGTGTGTGTGTGTGTGTGTGTACATATGTGTGTGTATATGCATATATACAAATATAT
ATCACAGCAAGTAACAATCAAAAAGTTGATAAGAAAATAGACTTGAACCTATGGAATAAGAAAGCTAGAA
TATCTGAAAAATAAGTATTCAAATAATATTTAGCAAATCTTTTATTGGTTTATGTTTACATTACTGTAAT
GTACCAAAGAACCCAAAAAACTACAATTGTAGAACAGAACTTAAAAGGCCACTTCCAGAAAAAAAGACTT
TCCTTAGTGTTTTCAGCTTTCTTAATTCTTGGGCACAAAATTTTCTAAGTCTTTGCGCACTTATCCACAA
CCACTAATTACAATATAAACTAATATAAAATTATGTCCTCATCTATGAAATTTAATATGCCATTTTCTTA
GTATAAATTTATTTCTAGAGCATGTATTATTAATGTGGATCTTTGTAGAAAAAAATTGTGGTACAATGTT
TATTACTACATACCCTTATTTTTTTAAATTGTCTTATCTTTCATATAACTTTTTGCTGATGTCATCATGT
GTAATAACATGATTTTTTAAAATTTTAATTTAGAAAAAATATATTCCTCTTGTATAAATGAGAAAACTAA
AGGAAAAGAGTATCGAAAGGAAAACATTATAGAAATATCAGAATTTAGAATCCCAATTCTTAAGATTTTT
TATTTTCTGACTACTTCAAATAATATCGTTTGCCTTTCTCATTATTCATGGGTCTTCCAAATATAGTGTC
TGGCCTTAAAAGTTGTAACACAAAATCTTCTGTCTGAAATGTAGTACAACTGAATGAAATTTAAAACCTT
CACGTAATATATGTCATATAGAACTCTCATAAAATATCAGTATGATGTGATAACAAAGGTCAGAACAAAT
GGAAGAAAAACCAATGTATGATTTCTGAAAACCTTATCCTGGGAAATGAGAAACAGACTATAATTCTTTA
CTTACATCTACATTATGCAAAAGTATCTTTGATGTGAAAAAATACTCAGTGTTGTTCATTCGATGTTTTA
TATGTTTTGGTAAAACATTTTGTGTTCACCCTTTTAAATATTCCAAGACACACTCACTGGCTAGAATCAA
TGGGAGAGAAAGAGGTGACTCTATAACTAATATAATGTTAAGGAAACTGCACATATCTTGGTATGTTTCT
GATAGTGCATAATTCCAGAAAATGTCTTAAGCATTGCATAATTGTTAGAAACATGAAATCTGCAGCCAGC
AAGCATGAGTTCCACATCTGACCCTACTCTTTCCAGTTGTGAGAACTTATTTGTATTTTAAACTACTGAA
TTTCAGGATGCCTCTGTTTATTCCTGTTTCTTTATTTTTTAAGTTGACAGTATTTGTACATATTCATGGG
GTACATAGTGATATTTCAATACATATAACATATACTGATCAGATCAGGGTTATTAGCACACCCATCATCT
CAAACATTTAGCATTTCTTTGTATTGGGAACATTCAATATCCTCCTCCTACCTATTTGAAGTTCTATAAT
ATTGTTAAGTATAGTCATCCTACAGTGCTATAGAAGCTATAACTTATTTCTCCTGTCTAGCTGTAATTTT
GTATCCTTTAACAAATCTCTCCCTATCTTCCCCAGCCTCTAGTATCCTTTGTTCCATTTTTTACTTCTTC
CTCATGTTTTAAAGGGAAATGATTACAGTATCTATTTCATTAGGTTGCTATAAGGAATAAATGGTTTCAT
AGCTCTTAAAATAGTGCTTGACAAAAGTAGGTACTATGTGTAGATTTTTATTAAAAAAAGTAGATACTAT
GTGTTATTATTATGACTAATCTTCTCCCAAAAATATGTTTGGTAAATCATTTCATAAATTTGTTTATTCT
TAATTTATTTTGAATTAGAATCATTTGTATAACAAGTTGGTATATTGCTGAGCACATCTGTAAAGTAAGA
CCTGAATATGGATTTGATAATTGAAGTTGACACTCATCATAGAGATTTATGAAATATTGTGCGCTTGATT
GCAGTAGGACAACTTACTATGGGGAAGGAATCTTGTAAGTATCTTGCCTGGCCAGTATACATGGAAGTTG
GTACACATGGAAGATATACCATATGTATATATCCCGATATTGATAATGTTACTTTCCAAAAGTGGAGGCA
CTGCAAATATGATGAGAATGTGACTAGAGCACGCACTTGTTTGTTCTTTCTGTCCAGCATTAATTTCCCC
CTTATTCCAGTAATAGTAACTCAACAACTCTGCTCTTTGTCATTTCCTGGTGTTTTTTTCTTTTCTTTTC
TTTCTTTTTTTTTTTTTTTTTTTTTTTTGAGACTGAGTTTCACTCTGTTGCCCAGGCTGGAGTGCAGTGG
CATGATCTTGGCTTACTGCAAACTCCGTCTCCTGGGTTCAAGCGATTCTCCTGCCTCAGCCTCCCCAGTA
GCTGGGATTGCAGGTGCCCATCACCATGCCCGGCTAATTTTTTCTATTTTTTAGTAGAGATGGGGTTTCA
TCATGTTGGCCAGGCTGGTCTTGATCTCCTGGCCTCAAGCAATCTGCCTGCTTCGGCCTCCCAGAGTGCT
GGGATTACAGGCATGAGCCACCGGGACTTGTCTGTCGTTCGTTCCTTCCTTCCTTCCTTCCTTCCTTCCT
TCCTTCCTTCCTTCCTTCCTTCCTTCCTTCCTTCCTCCCTGTCTGTCTTTCTTTCTCTTTCTTTCTTTCT
TTCTTTCTTTCTTTCTTTCTTTCTTTCTTTCTTTCTTTCTTTTCTTTCTTTCTTTCTTTCTTCCTCTCCT
TTCTTTCCTTCCTTCCTTCCTTCCTTCCTTCCTTCCTTCCTTCCTTCCTTCCTTTCTCTCTCTCTCTCTC
TCTCTCTCTTTCTTTCTTTGTCTTGCTCTATCTCCCAGGCTGGAGTGCAGTGGTGTGATCATAGCTCACT
GCAGCCTCAAATGCCTGGTCTCAAGGGATCCTCCTGCCCTGATCTCCCAAAATGCTGGGATTACAAGCAA
AAGCAACCTTCCCAGCTCCAACTCCACTTTTCAGAAATGCTTCTGTACCACAATGATAACCCTGACTACT
CATAGGTTTCAAATGAGGACTCTTCCGTGTTTCAAGGCTACAGCATGTGGCTTAGGAGAGGCCAATCAAA
GACGGCATTACCCTGGACAGAGTAATTACTTCAGGATGGGCATGTGACCCACCAGGTGTCAGTGAGATGC
AATGAGTTTTTCGGAGTCTTTCTTCCCACTGAATTGAGGTCTGGAAGGTTGGGAGGCTAGAGCTGCTACA
GCATCTAGCTGTAGGGAAGCTACGGCTAAGAGAAGAATCTTTAGGAAACTTATCACTATGAAAGTCAGAA
CAAAAGGATACAGCATAACTATGAGCTGGTAACATCAGCTGAGCTCTTGAATCATCATATGCCTAAGACT
AGCCCTGGACAAACCATGAACCAATAAACTCCTGTCTTTATTTATTTTTTACTTCAATTAGTTTGAGACA
TCTTTTTCTCTTACAATCAAAAGTTATCTGCTAAAGTGACTAAAATATTATTTTGGGCTACATCTTCCCC
TTGCTCATTTCCAACAAACACATTTTCTTCTTTTCTCCAAATCCTTTATTTTAGATTCAGTGGGTACCTG
TGCAGGTTTGTTATGATGGTATATTGCATGATGCCAAAGTTTGAGGGCACGATTGAACCCACCACCCAGG
TAGCAAGCATAGTACCCAATAGGTAGTTTTTCAACACTTGCTTCCCCCACCCTATACCCACTTATAGTCC
ACGGTGTCTCTTGTTCCCATGTTTATGTCCATGTGTACCCAATGTTTAGCTCCCACTTATAAGTGAGAAG
ATGTGGTATTTTGTTTTCTGTTTTTGCCCAACAAATACATTTTCCAATGAAAATGATGAAGCCAGAAAGT
ATGAAATACATTGATTTAACAATTAGAACTGTTCTAATTATAATTGATACCGGTGCCTGTTTTCTTTTTA
TGATTGAGTAATGATCACAGATTTTTGCTATCAGATTTTGAGTTTGAATCCTGCCTGTATGATCTTAGGA
GACTTTTCCTAATCTCAGTGTATGTCCTTACTTCAGTTTGCAAATAAGGATAATGAAAGCACTTATCCAA
TAGGGTTGTGTGGAGGATGAAATAATACAGGGCAAATGTCTTAGCCAAGTGACTGGCACTTAGTAAGTCC
TCAAATCTTCACTATGATATTAATTGAAAGCCTAGCATTCACAATCTCACTGGTTTATCAGTTCTTTTTT
CTGGTTGCTTTTTAGTGACTTAAATCAGAGATAAATAAGATTGGTGTCTCACTGGGTTAATATTTTTTCT
GGAGAGAGTTAACTTGTGTGGCACAGGCCATCTCTTTATTGAATTAGCAAAGAACTACATTGGATTTAAG
ATTATCTGGGGCTGAGGCTAATATCAAATCCTACTCTATGTTTGTAGTTCCTTGGCTACAAGCCTACATA
CGGTAATTGCTGTTTAATATGAATTTTTATTTTTAACCCTTACATATAGTAAGGGTTAATTGCTATTTAA
TATGAGTTTTTATTTTTAACCCTTCCCTATTTACGTCATGCTTTGCTTAAGGACCAAGATATATTCTGAG
AAATGTGTCATTAGGTGATTTTTGTCATTGTGTGTACCTCATAGAGTGTACGTAAAACCTAGATGGTAGA
GCCTATTACACAACTAGGTTACATGGTATAGCCTATTGTTCCTAGGCTACAAACCTATACTGGATGTTAC
TGTACTGAATACTACAGGCAATTGTAATGCAATGGTATTTGTGTATCTAAACACGTCTAAACATAGACAA
GGTACAGTAAAAAATATGGTATTATAATCTTATGCGACCACTGTCATATATGTGATCTGTCATTGACCAA
AAATGCCATTAAGCAGTGCACTACTGTACTTTCTCTTTACTTTAAATTTGAATTCTTTATATGGGTTGAA
TGACTTTCTGACATAGCAAATAAAAAGCATGAGGAGAAGCATTATCTGTTAACAAAATTAACACTTAAAA
TCAACAAAGTTTTAATGTTTCGTTCCAAGAAAAGCCTGTGGAAGATCAGTTCCACAACTGAGAGCTTTGG
GCTGCTTCAGACATATGTCTGTGTGTACGCTGTGAAGGTGTTTCTCTTCACAGTTCCCCGCCCTCTAGTG
GTAGTTACAATAATGCCATTTTGTAGTCCCTGTACAGGAAATGCCTCTTCTTACTTCAGTTACCAGAATC
CTTTTACAGGAAGTTAGGTGTGGTCTTTGAAGGAGAATTAAAAAAAAAAAAAAAAAAAAAAAAAAAAGAT
TTTTTTTTTTTTAAAGCATGATGGAATTTTAGCTGCAGTCTTCTTGGTGCCAGCTTATCAATCCCAAACT
CTGGGTGTAAAAGATTCTACAGGGGTAATGTTTTATTATTCTTATTATGCTTATTCTCTGTGATGCTTCT
CTACCTTTACAGTAGTAGAATCCTTGGGGAAATCTGCAGAGGGACCACTTTCATTTTGAAGCTGCTGGCT
GCATGTTTTAGCATGTCTCTTCTATTAGAGAATCCAGGCATGGCAGTTTCCTCCCCCAGTGTGCAAGGAC
CATCTTCATGCCTATGTCTGTCGCTAGGCATGAGGGTCTCTAGGAATGGGTGAAAAAAATGAGGGATGTT
TTGGAGGCACTATAATACTGGGGAGGGCAGTCTGCTAGCTGGTAGCTGAAAGGTCCTGGTTTACTTCAAC
ATTTTTTTTAAATAAAACTGTGCAGTAGTTTTTGTTATTTTAGGTTTCCCTCTGTTTTATCTGGTGTATG
CTGCAGAAGTGAACTGCATAACACATTTCACTCTTAGAAAATGCATTTCATATATTTAAATGACAACTCT
GACTCCTATAGTATGGGGGTGAAATAGATTGATTTTTTGAAAAGATGGAAAAAATAGATTAAGTGTCATT
TAGGAGTCAAGATGTTGATTGTATTGTTTCAAATAGCATAGAAACTCCTGCCACTGGTTCAGAGGTAACA
TTTGGAATGACTGATGTGCTCTTTTGAGTTATCTACTTTAGTCTGGTGAAATATGATTTGAATACCAGTA
AAAATCTATTTAGGTTATTGCAAAGACTCATATAATAAGAGTTTGCTTGAAAAGATAAACTATCGTAAAC
TTATTATTTTTTCTCAAGTCCAAATTCTGATATGGACACTAAGACTGTGATGCTACGTTTTTATGAAATG
GGCATAATTTGAGGAAATTGGCTATCTCTACAGTATTTCCTAAGGTACTGCAAATGTGTTAATTTAAGAA
GCAATTACACTAGCTAGCAGCAGGTTGCTAAGCCAGGGGGTCCGAGCTACAGACCTCTGATGTGGATGAC
TTCCTGCCCGGTTTGGCAGGAGCTGGGTCCCTTCCTGTGCAGCTTCCGAGCAGTTCCAAACACAACCAGG
CATCTTCTGCTCCTTCCCACTCCCTTTCTTTCAAGCAGGATCTACCTATGTTTCCATAGCCTGGCATTTT
AGGATTTCCAGAGATTTTTACCCAGTGAATTGACTTGGAATAAACTCTTAAATGTGATAAAGTGAGCCAA
AGGGCACGTGCAAAGACTTGGAGAGATATGTGCAAGATCCTCAGCTCAGGTTCTACAATTTAATATGATG
TTTCATCTCTTGTTTTGGATCTCTGCTATTTTTTCAGAAAGAATGCTGAAAAAGATGGCTGCCAAAATAT
TCACACAAGTGAAACAGCAGGCACAAACCAGTATCCTCCATTTCTGAGCTCTGAATTGCATATTTTCCCA
CAAGTAGCACAATCACTTAAAACATTTTGGGAGTGGGAGATTAGATTACCTCATAGACACCTACTGAATG
TGAGAGATAGATCTCCATGTGAGCAAATGGATGCACATATGAGATTCCCTGAATACCAAATACAGCTTTC
TTTGAAAATATTATAGCTATCTGAGTTTCTATGCTGTATCTCAGTGCTCAGTGTCATGTAACTGACACAA
TCACCTTTTATTCTAATGGTCATTGCTTTTTTTCCCTCCCTGTTTCTGTAGCACTTTCTTATGCAAGGAG
CTAAACAGTGATTAAAGGAGCAGGATGAAAAGATGGCACAGTCAGTGCTGGTACCGCCAGGACCTGACAG
CTTCCGCTTCTTTACCAGGGAATCCCTTGCTGCTATTGAACAACGCATTGCAGAAGAGAAAGCTAAGAGA
CCCAAACAGGAACGCAAGGATGAGGATGATGAAAATGGCCCAAAGCCAAACAGTGACTTGGAAGCAGGAA
AATCTCTTCCATTTATTTATGGAGACATTCCTCCAGAGATGGTGTCAGTGCCCCTGGAGGATCTGGACCC
CTACTATATCAATAAGAAAGTGAGTTCTTAGTCAAGTTGCCTTCACTGCCTATTTACTAATTGGTTCTGG
GCTAGTCCCAGGGATGATGGTGAAGAAGGCTGGCCTCCTTCCCTCTGTCTAAAGTATCACTAAGATGCTG
GATGGGCCTGACCGTGTAATGGACCAATGATCCTAGAAGTCTTTTGGAAGCACTCATTTGAACCTGCATT
TGTGAGACAGGCAGAGAACTGGTGAGGCATCCTCCAGCGCGGGAATTAAGGAAGGACAAAAGCCTATTCA
CCTTCTTGAATACAAATTATATGCTTAAACCAGTGTAAATTGACCCTGATTCCCTAATAATGTTGAGAAG
CAAAAACTGTAAACTAGGAGTCTATTTAAATTTTATTTTTTATATTTGCAGGAGTAGTATCTAAATTCCT
CTTTATAGTCTCTAGCTCTCCATAAGTCACTTTGATCTTCAGTGGGTTTAATTATTCCTTTATACCATAC
TTTCTCCTTTCTATTGCTCTCCACAGAAGGAATAATAGCAGGTGACTTGTAGGTGCCAAATAAGATTCTG
AGCAAAGAACACACCTGGAAAACCTTGAAGTTCTCATGAGAAAATTTTCTAACCAAAAAAAAAAATCAAA
GCCTCAATTTTGTGCTTTATGTGAATTATAAATGCGGTTTTAAAATACTTACATTAAAACTTGATAAAGT
TGCTAAGAATTCCTATGGCATTGATCACAAATTTTCTTAATAATCCTCATGTCATTTATCAAATTTAGGA
AAGTTTATAGTGCTCAGAAAAAAAAAGCATCTATCTTCATGTCATATGATGGTAATTATTATGTTATACA
CTATTTTACAGGGCAATATTTATAAATAATGGTTTTACTTTTCTCTTAAAATATTCTTAATATATATTCT
AAGTTTTATTTTATGTGTTGTGTTTTCTTTTTCAGACGTTTATAGTATTGAATAAAGGGAAAGCAATCTC
TCGATTCAGTGCCACCCCTGCCCTTTACATTTTAACTCCCTTCAACCCTATTAGAAAATTAGCTATTAAG
ATTTTGGTACATTCATATCCTTTTTCAAATCGTCACTTAATATGATTTTCTTCTTTGACCAAGTTATTGA
GCTACACATTTTCCAAAATATCTGTGGTTGGCAATGTTATGTGTTCTTTCTTTTTCTTTCCTTTTACTCA
ATCGTTAGCATGTTGCAAAATGAGATCACAGGTAAGTGAATTACTTTCCCCCGTCTTCTAAGTGTTTCTT
CTCTACCCAACTCACTATTACTTCTTTCTTCTCTTTTCTTCTCCCTTACGAATTGCTTGCCACATCCCAA
GCCTTTCTCATTAATTTTGACCATGTTACCAGGCTTTCCTCCTGTAAGTCTTCAATTTACAATGTTAGGT
AAGGGAGTAAACTCCATGAGCTAATTTTCTTTACTGCTTTTACATTTGGAAAATAAATATACATAATCTG
AATTACAATTTTGTATGTTTTTGGTCTGAATTTTATGACTTTCTTCTATTTTAGCATTTAAAAGCTTTGA
GTTAGTAAGCGTTTACAATTGTGTCTGTAGGTATAACACCCTTTCAAATACTTTTCCAAATTTGTTTCGC
AACAGCCTTCTTATTGATCTTTTGCTTTCTATTCTTTTCCTTTCTTTTTCTTTTTATCACTTGTTCCTAT
GTTTTATTGAAGTCACAAGTCTTGCTACAATTATCCCTCTCCAAAGGATTAATGTCTATCTATCCATATA
CATTCTCATTTTATTTTTTATACTCCTTATTGAAGCACCTGCTTTCCAAAAATGAGATTGATGACATCTT
GGTGGGAGATGGCAATTTGATTGATTCCTTGAAATTAAATAGAGTAGTTGGAAATGAGAGATTTTATTCT
AGGCCAGAAACAAGTCTTGTTGACAGCCAGTCTTGGGAATGCCAATGAAGCAAAGGCTTGGAAGACTGAA
GCTGTGTGGGGCAGGGGCATTTACACGAAGAACACAGAAGTCATGGGAGGAGGAGTGATATGCTTCAGGA
ATCAGAGGTAAGGCAGGGGAACTGAAATTAAGCAAATCCTCAGATTAGGACAGCATGTTATTTCTTCTTC
AGAAAGAATCGTTGCTTGGAATTCCATGATGTGGTTAGCCCAGGGCCTGGTATTAAGGCTTTCAGTATAA
ATATTCTCCACCTTTAGCAGGCCTAGAAAATATTTGAATTAGATAAGGTATGGAACTAAATTAAGTAGGG
AATTCAGGAAATGGGATAAGCCTGGTACAGGGTACTTATGTCATTTCTGGGTGGAGGGGATGTAAAGCAT
GTCCCTAGACTTGCCTCCCAGTATGGCAAATGTTGTCTTTAGAAGTGTAACATTCTGTAAAGTCTCCTTT
TAACCTCTAGGTTGTTCCTCTTCCCCAGTTTAGGTGGATATCAACATCTTTTATTTGATGTTTATGTTTC
ATGTTTTAAGATTTCCTAGTTTCTTGGCATTACCTTAAGCAATAATGTTTTCTTACCTCTCTCTGTTTTC
CAAATAAGAGAACCCAGTAGCATGTGGGGAAAAAGATGTCTTTGAGTTAGCATTAGAAATAAATAATAAA
GTTGGAATTTATATTTGGGTCTCATGATTATAAATTATGATCTATTATTATGTTTCAAGCATTTGTAATC
TGTGCAGTGAATAAATCTCTGCATAAACTATTCATTATATTTTAAAATAATTGTATGTTCCTTATGCAAA
CGTAATTTATATATAAAATTACGTGGAAAATTCTAGCCTAGAACTAGACTTCTGTTCCTAGTAGACATTG
GGAAAATATTCATTAAATAAATAAGTGACTAGTAAGTCAGAGATTAGAGAATCAGATACAAAAAAAGTGA
AAAAATAAGTTTGAATGGATCAGAAAAAATCTTTCTTGTCGTGCATCTGAATGATGAGATGGAGTTAAGA
AAACCCAATATATTTGTTTTTTACAAAAGCAGATTTTTGTTTTAAAACTTTTGTAATAGACCATGGAAAA
TCTCATGAAAACTATTGTCCCCACTTGAAAAAAAAAATCCTAGGAGATTATGAATCCCCATTTAAAACTC
CCTGGAAAAGAGACTCCTGGTGGTAGAGGGTAAGGGCAGTTTAAGAAATTCTGATCAGAGAATATGAGTA
CTAAGGACACAGTTTGTGCCAGGACCTCTCTAGATATCCAGATACAACTTGAATCTGTGGGCTTGTATTT
GCTTCCTGGGGGAAAGAACTCACCCTCCACAGGCTGAAGTCATTGGGAGGCCTGAGGAGATGCAGGTTGA
AATGTGAAGCCAGCCAGGCAAATCTATTAAGGTATGCCAAGTTGAGCTGTTGATTCTCTGAGAAGTTTAC
AGATGGCTTAGAGTCACAAATTTATTCCAATGTAGAAAATTAGATTTTAAAAAGTCTCTAATTTCCTGAC
TTAAAGTGTTATATTTCAGATGTCTCACCTTGGAGCAGAGATATAACAAAAGGCAGTGAGGCTAATAGTC
TAAGATACATGAATCCTTCCATGTTTTGGTGATGCTGGTGCAATTGATCAAATAGCCCAGTAAAGTTAGA
GGTATATAGATGCTGTAGTTAGTAACTGATTTTCACAATAATTTTGTCCTTTATTCCTCTTGTTGCAAAC
CCTAGACTTAAATCCTGATTTTCTGACTTCAAGTACAGTGTCTTTTACTGTAAGTTAAAAATGCTTGGAG
AGATGGTCATGGTTGTTTGGCCACAGTTGGGAGGTCATTGTATATTTATTACCACTAGTTTATAAACCAA
CAAGGAGCCATTCATGTTAAATAAGTTTTTATTTTAAACTTGGACTAATACCTCTATTTCAAACAAAAAC
CTTGACTTGTTTCTCAAAGAGCTGTTATCTATTAGGAGCTATTGTGTATCAAATTAGCTTTTTTAAAAAT
TTATTTTGGCTGAATGAGAAATTATGCTTGTGATATTTTTACCAGGGTGCATTCTGAAAACTGAAAATTC
TTTTGATGTGCCTAGTGTCTTATTTGATATTTAAATAAAACATGATTTATTTTCTAGATAACAAACAAGT
TAAAAATAATCTATGTTCCTAAAGTTCCCTACCAAGCTTTTAAATGTGTTTCCTGTCAGCTTTTATTATT
TTAAGTTAATATATGCACACTCCTCTAATTTATTTTGCATTTGTTACTCATTTGTTCATTTGCAAGTACT
TACTGAGTATCTACCATGTGGTAGATATTCTTGTAAGCATTGGGATGCAACATTGAACAAAGTGAAGTTC
CTACTCTCATGTAGTTACATTCATGTGAGTGTGTGTGTGTGTTTGGAAGAAGAAAGACAATAAACAAATA
CGTCGATTGGGAGCTAGTGATAAGTGTTACTAAGAAATATAAATTAGTGTCAAAGGGAGGAGTGACAAGG
TGTTGTTTTAGATTGTATGGCCAGTAAAATCCTTTCTGAAAAGGTACCAGTTGAGCAGAGATCCGAAGGA
AGCAAAAGAGTGAGATATGGGAATCTAGGGATAAAGTCAGTTCAAGGAGAACAGCAAGTATGAAGGTAAA
GTCTGAAGGTGGTGGTGCAACTAGTATGTTTAACAATCAGCAAGGAGACCTGAGTGGCTGGAGCAGAGTG
GGAAAGGCAGGAAGCGAGAGGATGACATCAGTGGGAGTGAAGGTCAGGGGCTAAAGTTGTAGGCGAGGTG
CAGTGGGTCATTGTGAGGACACCACATTTACTCTGAATGAGACTCCAGGAGGGTTTTCAGTAGCAGGATA
TCATGACTTGACTCACATATTTAAAAGATCACTCTGGCTGCTTCATGGAGAATAGACTGCATAAGGGGAA
AGAGTGGAAGCAAGGAGACTGGGACCGATTGTCATTATGAAGGCAAGAGAGCATGGTGGCTTGGAGTATG
GTGGAAACAATGAGGATGGTAAAATGTCATCAAATTTTGAGGTATTTTGCAGAAGAGCTGACAGGATTTG
CTGAGCAATTGGTTGTGGTGTATAGGAGGAAGGCAGGGACTAAGGATGATTCCAAGCATTTAGTCTGGTC
AAGAAAGAAAAATGGAGTTGTGTTGACTGAGCTTGGGGCAACTTGAGCAAACCAATTTGGTGGGTAAGAT
CAAGATCTTGGTTTTGAACATGTTATGTTTGAGACACCTATTAGACATCATTGGAGGAGTTGAGGAGTTA
GGTGGCTGTGCAAATCTGGAGTTCAGAAAAGGGCTGGGCTGGAGGTGTTCAACTTTGGGAGCTGTCAGTG
TACAGCTGGTATTTAAAGTCATGACATTGGACTAGGCTGCCAAAGAGCTGAGACCCTCCCTCCAAATCAC
ACTAGTAATGCTGAACTACCTATCATTTGAAAATGGTAGGAAAATGGAAAACATAGGTTTTGGTATCAGA
AAACGTAGATTCAAACCCTATCTCTAAAATTTACTTTTTAGCTATATGATCTTAGTCCAAGTTACTCCAA
TTCTTTCGAATCTCAGTTTCTCTATCTGTAAAATTATAATCACAGCTTAGACATTAATAATGATAAAATG
TATGACAAGTATCTAGCACCAGATCCCATGCTAGTACTTAGTAGGTACTCAATAAAGGATATCTATGACA
GTAATAGCTAAAATTCTAGCAGCAACTGCTGTAAGATTAGCAAAAAGGAAACTCTCATATTCCTTAAGGA
ATTGCACAAAGAACTTTATAGAAATCCCTACTCTGACTCTGCAAACAAAATCTTTATATAGCACCAGAGT
TTAGACCTGCAACTGACCCAAACAATGTGGTCAGTTCTGTCTCATTTTGTAGATGAGTTCACTGAAACCC
AGAGATATTTAGTTTTTTCTAAGGCTACATTTTCTATCAGTGGCAGAGCTAAAACTTCAGACCAGGTTTT
TTGATTCTTGGCTCTTTGCATTTTGCATCCAATAGAAAACAAATGATTTTTAAACCCTCGGATTTAATAT
ACTTGGGGCATTGCCAGTGTTCTTGTTTTATGCATTTCAAAGGTGCTTCTTAGTTGCTCCAACTTACTGA
TTCATTAAATAGTGTCCATACTGAGATATAAAATATCATGGTTTTCCATGAAAAGAAATATACAGGTTTA
TATGAAAGCAGATGACACAACAATTTCTCTTTCTTTTGTTTTCAATGCTCATATGTTATCATTTAGTTAT
CTACTGGCAAATAGGAGTTTGTTCATATTAAAATTAAACAATCCAATATTTAACACTGTATATGTGACAT
TTACTCGATTTTTCTGCTGGCTCAGAAATATGCACTGGTATGCAGAAAAAGACCTATTCTATTCTACTTC
AAATTATCCATTTTTACATTAGAAAACCTCTAACATCAGGCTATCTTCTACTTCTAGTTTATATATAGGT
TAAAAACTCCTCTGCAACTTCTCTGGATATTATACATTATTACAAAGTCTCTGAACAGAGCATAATGTCT
TTTCCTTCCTATAGAATAACAAAGAAATGTCCTATAATTTTATACTCTATAAATGAGTTATTAATGGTAA
GAAACCAATAATTATTATCTTAGTGGATAATGACTGTATACTGTAAGAAAAGTATTATCCACATTTATAT
AAGAAAACTGAGCCTCAAAGAATTAAACAAATTGCTGAAGCCCACATGGCTGGTAAGGGATGTATCTGAC
CATGGTTCATTGCTCTAAATCTCATGGTGCTTCATCCTCGCTCCACGGAGACAGGGGTGGGTGTGCCAGT
GTTATGATGATCCAGGCTCCATGTCAAGGGCTACTTAAACAATTTTCACTAAAAACTTGAAGAAGTGTTT
CTTCATAATATACACAAAGGAAATATTTTACATTTGCCAACTCGCAGGTTAGTATCAATCAACAGGTTTA
CCCACTGTTATGTATACCTGGCATAAAGAAATTAATAGATTAAAAAACATCTTTGTCCCCTGATATTATA
AAAGGTTTATCTGCCTCTATTTTATTTTACATTGAAAAGTTCTTAAAGCAATATTGTTCCAGGATACAGT
GTTCTTTTGAAAAATGTACTCTATGACTTGGATTACACATTTAAAAAATAATATAGGATGTATGCATTTT
GCTACTAGTTTGAGCCTTTTGAAATCTGCTTTGACGTGGGGTTTCTATACTTTTTTGATGCATGGCATCA
CCAATGCAAAATCCATACCTACATTAAATACTTTTGTATTTGAGTTTTTGTTATTTGAGTTTTTTTTTTT
TTTTTTTTTTTTGAGACGGGTTCTCGCTCTGTCGCCCTGGCTGGAGTGCAGCAGCGCAATCTCGGCTCAC
TGCAAGCTCCGCCTTCCGGGTTCACGCCATTCTCCTGCCTCAGCCTCCGGAGTAGCTGGGACTACAGGCG
GTCTCGATCTCCTGACCTCGTGATCCGCCCGCCTCGGCCTCCCAAAGTGCTGGGATTACAGGCGTGAGCC
ACCGCGCCTGGCCATATTTGAGTATTTTTAAGATCATCTGAAACTATTTCAGTCACTCACCAGAATCCAG
GAATTTGTAAAGTATGTGACTGATGAAATAAATTAACAATGATTTAGAAACTTAGTGAATTTTAAGCCTT
TCTATTTAGAGATACCTATCAAACCACAAGCGTAAAAACTTGACCCTAGTTATCTACTATTTTTCTATTA
AAAGCAAAATTGTTCTTTTTATGTATCAGAAGTTTTAACTTAAGTGTATACTTTTATTAAAATGATAGCC
ATGAAATAAGGAAAATGCCTGTTTTCGACTTATTATCAGTGACTAATTAGAAAATAATTATTTCTCTTGT
TAATGTTGAAATATATATTTTACTTTTTTATATATAACTAAATTATACCACTATAAAGAGTAAGTTTTTA
AGTGTCATAAAACCATTGCCGAGTCCATAATGCAGCATAATTGCATAAGGCTGTTAATTTCCACCTTATA
TTTTTCTTATATTTTTACCCTCAAAAAATGTAGAAACTTGTGTAAACAATATGTATATATATTTTAGACA
GAGTCTCACTCTGTCACCCAGGCTGGAGTGCAATGGCGTGATCTTGGCTCACTGCTACCTCTGCCTCCTG
GGTTCAAGTGATTCCCCTGCCTTAGCCTCCTGATTAGCTGGGATTACAGGCATCCGTCACGCCTGGCTAA
TTTTTGTATTTTTAGTAGAGACAGAGTTTCACCATTTTACCCGGGCTGGTCTCAAACTCCTAACCTCAAG
TGATCTGCCTGCCTCAGCCTCCTGAAATGCTGAGATTATAGGCGAGAGCCATGGCACCTGGCCAACAATA
TATTTGAAGACAAACTTTATGCTGTATTTTTAAATAATTTATCAGAAATTGTTTTTAAAAACTCCATTTA
GTAACAAATGAATTGCAAAATTAATTTCATTAGTCAACTGACACTGTGAAATAGCAAGGCTATAATGGTG
AATAATATAGACATGATTCCTGTCCTCATGACGCTCAGAGAGTAGTTGAGAAGATCATCATTAAAATTTG
TCATTAGAGGAATAATATAAGGGCTGCTGGGGTATATAACTGGTAATAGGTCAATCTGAGTACTAAAGAA
AAAGAGAGGTGACATTTCCAAGACCCTAAAGTCAGAAACAGCATATAAAACATTCATAACATTTGACAAC
CTAAAATAATTACAGTATTATCCAAATGGGGATAATGCAATGAAGAGAAAAGAAAATGAAACTGGAGAAG
TAGACAGGGATCAGACCTCCTAGCGTCTTGACTCTGTGTTAAGACATTTGATCATCATCCTAAGAGTAAT
AGAAAGCTACCAAAATGATGCATATTACATTTACAATGGTCGTGTTAGCACAGTATGCAGAATGGATTAA
ATGGAGCCAAACATGAATTTGGAAACATCAGTTTAGAGGAGACTGCAATAATCTAGATGGACTATTAGAT
TTGATGCTAGTCTTGACAGACACTTGGACCATGCAGTGATATGGGGATGGAAAAAAGTAACTGAATCCAG
AGATAGCAGGCAGAATTGACAATATTTGATGGTTAATTAGATATGAACGTTTAAGGGGAGACAGAAATCT
AAGATTTCTCATAGGTCTCTGGCTATATTATGCACATTTTATAAGACACAGAGACGTCAAAGGAGTAAGT
AATTAGCAGGAATGGAGGGGTAGATTAAAAGATACTTTTCAAAAGTTCAGTTTTAGAATTCAAAATTTGA
AGTGTTGATAAGATATGTAAGTACAGATGTCCTATGGACAATCAAGTATGTGGAATTCAGAAGAGCGGTC
TCACTTGGAGAGAAGTATCTGAGAATGGTGGGTATATAATGGTTATGTTTGTTGAGCAATGTTTGTTGAT
GGACTACACTAGGATGAGGAGAGTAGAAGAGAGGTAGATGGCTTACATACCTTATGTCTTTCTTTTCAAA
AAGAAAAATGCCACATTTCAAAGAATACAGAGAAATTGGTGCCCTGAGGCATGAAGAAGCCAGGGAATTG
GAACCTCCTGAAAGCAGAGGGAAGATAACTTAATGCTAAGGGGAGGAACCATCCTTGTTGAATGCTGCTT
AGAAAGCATGTCAAATATAATCTCAAATTATCCTTTTGTTTTTAGTGACAAAACGTGAAGATGTTGCCAT
CTTTAGAAAGAAAAGCTGGTAGGCTGAATATATGATAGATATATAAGAAAAGGAAAGATAAGCAGCTCTT
TCAAAAAGTTTGGCCATGATATGGAAAAGGGAGATAAGGCTGTAGTAGTTAAGGAGGGTTGTGAGTCATA
GGAGAGAAATATCCTTTCCCCTATGCCAGGACAAGGGAGACGTAAGCAAGTTTGTGTTGCTGGGAAAAAG
CCAGTAAGGTGGGAGCCATTAAGGATAAGAAAAGAGAGAGATAAGAGATTGCTGAAGTCTTCTGGAATGG
GGAAGGTGTCCAGAATACCGGTGGAGGGATTGGAAACCTCCCCACTGTAACAGGAAGGAGGAAAGAATTG
GTCTCAATGTGGAAAAGTTTGTTGATTTGGGGGTGGGAAGTGGAGGCGGGGCATTGTGATGCTGTCTCTT
CTCTGTAAAGTAGAAAATAAGTTTTCAGCTTGAAATGGAGCCGGAAGAAAGAAGAGGGTTGGAAGCTGGA
GGAAAGTGGAGAATATTTGAAATTTTTCTTTGCAGAGAGTGGGAGATGGAGCCTAGTAGGAAAATACAGG
ACTGTGTTGAGGACCACTGAGGTTTGTGACCATAAATTTAGAATGGTGCCAATCTGCCACGGGGTGTTAT
TTTTCCCCAATAGGGCTCAGCAGACCAACAAGCACAGGGGACCCTCTAGTTTTATATACCAATAGCAAGT
CATTCTTTATTTAATTTAGTTTTTTGTTTGTTATAGCAATAAAGAAAAATTGTGTTTCTTTGAAATGGTA
TTTTGTGTGTGTGTGTGTGTGTGTGTGTGTGTGTGTGTGTGTGTGTGTGTGTTCTCTAGATTATAGTGCC
CTCACGTGGCTGATGCACATCATTTACTTATGAGTGTTTTCATATGGATGAAAAAACTGAGAATTAAGCA
TGGGCCTTACAGCACAGCTACTAAAATTAAAAAATAATAATTATTATTATTGTTTTGACTAAAACCAGTA
CTAGATGCAAATGAACTTTCTTTCTAGATCAACACAACATTGTCCAGTTGTAATAGTGTTGATATTTCAT
TATGTGTAAGTAATGTGATCATTTATAGTAAAAACATTAGGAGTGAGAAAAGATATGAAGAGCACGTATT
TCCTCTCTGGAATTTCTATAATTGTGTCCAGATTCACAATGATAAAGAGTGCCACTTTACATAATGGCAA
CTAAATCTTTATTATGCTTTTTATTAAATATGAAAGTCATTACTATTATCTGATACTGAATATTTTCTTA
AATAGTTGTTTTGGTTTTTGTCTTTTGCTGTTTTTTAATGTCAAGGAAAGTGAAGGGCATTGGCTATACC
TGCAAAGACAAAGTGGGATAGGGTGAAACCAACCCAATATTTGTAATAAACTGTGTTCTGTATGATCCAG
GAAGCAATTCATTGAGCATCTATTACGCACTAGGGCCATTAAGTTGAAGGAGACTTACATATTTTAACAA
ATTTGATCTTCATAGCAATCCTGTTACATGATTACTTTTTCTTACTTTTATAACCAAGTCAATTGAGATT
CAGGGAACTTAATAAGTAATTTTGCCTTAGTTACAAACCCCAGAAATGCCAGTGCTAACACCTGCCATCT
TCTCTCATAGTTCAGGATTTTATGAGCAATTACAGTATATTATACCCTCTGTTTAGAAAGGACCTTATTA
TAAGACATTCCACCAGGGTAACTTTTAGAATGATGTTATAATACATTTAATTAATTACTTGAATTGTCTT
GTTGAATTTTTGCCAGGGTTTACATATGTGCTGAACTTGCAGTTTTAATGTTCAGTTGAGTCTGTCGTTA
AGAAAATTTAAGTTGATAAATTATTCACTGATGAACTACTTTCTTTGCATTTAATCTTTTTAATTGCTAA
AGGTACCTAAATAGCCTCAAAATAGTTGATGGCTTGGCCTGAAGACAAGATCTAAATATGAGGTTGCTGA
GTTATAGAAATGGCAAAAAAAAGGGTCAATAATAGAATAATAAGCAACAAAATAATAGTAAGCACTAAAG
TTTTAAACTTCATGGTGGTGAAGGCATGGTAGTGCATAAAAGTAAGATTTTTCCATTGAACTTTGTCTTC
CTTGACGATATTCTACTTTATTCAATATGCTCATTATGTGCACGATTCTTACCAACTGTGTATTTATGAC
CATGAGTAACCCTCCAGACTGGACAAAGAATGTGGAGTAAGTATAAATATTTTTCAATATTGACCTCCCT
TTATGTTTCATATTGTGCTTTTAACACCTTGAGACCTCCTCAATTTCTTTAACAAATCATGCTAGCTACT
GTTAACCAGACCCTGATTCAAATTCATTTCTGTCACTAAATGTCTTCTAGGACAAAGCTTGTAGTGGGCT
CACTTAGTTGTGTAAATTACTGCAGTAGTTTGACTGCTATTATCTGCAGCCCTTTATCTTCTTTGTGAGT
CTTATGTTCTTTTGAAGATCACCAGTGATTTACTAATATCTACTGATAAAAAGTATACCTAGTTTTTATG
TTCCTTTTTTAATGACTACCACAGTTCTGTGTTACTAGTATATGTTTGATGGCTTTTAATGGTGCATATT
TTATGAAATACAAATGCTTCACTCATTTTTGTATTAATACCTATTTGCTCAAATCGGACTGAATGCCAGT
GTATTTCAGATTATGTCTTCATATAGAGCCACATTATTTGGATCCTTTAAATTAATTAATGTGGAAAATG
CAATATACATTTATTTACAGTCAATGAGAATGTCTTTTGGAATTTAATGTTTCTTTTTTGACTTAAGCCC
CACCTAAACTCTATATCGTAGGGGGACCAACCTGGAAGTGTCTAATTTTTGTTTGCTGTTTATGTCATCT
TTAAGATATGTACTTGTAAATTAACCACTAGATTTTTAATGTGAGCTTGGCTATTTTCTCTCAGGTATAC
CTTTACAGGAATTTATACTTTTGAATCACTTATTAAAATACTTGCAAGGGGCTTTTGTTTAGAAGATTTC
ACATTTTTACGGGATCCATGGAATTGGTTGGATTTCACAGTCATTACTTTTGCGTAAGTATCTTAATACA
TTTTCTATCCTGGAAGAGTAAATCACTGGTGGGAGCCTATACTATATTTTCCTTGGTGGCTTGCCTTGAC
AGACCAAGCATTTTTCTTAGTAATCATAGTTTTCTTCCAATCAAATTATCCAGTTTGGAGAAATTAGGAA
CTATCATAGTAAATTACATGGCTTTGGTTTCAATTAGCACTGTAAAGTAATAAAGTTTCCCAAATAACAG
AGATTATGATTGATGACAATGCCATTTTCCTCTTAATTGGGAAAGCTGATGGCGACACTCATGAAATTAA
AAAGGTCTTGATGAAAGACCAAGGAAGACGTAGATTTCCCTAAATTCTGAATAACTCTGATTTAATTCTA
CAGGTATGTAACAGAATTTGTAAACCTAGGCAATGTTTCAGCTCTTCGAACTTTCAGAGTCTTGAGAGCT
TTGAAAACTATTTCTGTAATTCCAGGTAAGAAGAAAATGGTATAAGGTGGTAGGCCCCTTATATCTCCAA
CTGTTTCTTGTGTTCTGTCATTGTGTTTGTGTGTGAACCCCCTATTACAGATATGTGACAGAGTTTGTGG
ACCTGGGCAATGTCTCAGCGTTGAGAACATTCAGAGTTCTCCGAGCATTGAAAACAATTTCAGTCATTCC
AGGTGAGAGCTAGGTTAAACACCGAGGCTGACTTTAGCTACAGTGGTGCTACAATCACAGCTTTTGTGCA
GAAGCCTTGTTGCTAGTTGCATATTGCAAATAAATATGTAAAAAAGCAAGAATTGGTACATCATTTTTTG
GATGGATTTGATTCTTTGCTTTTTACTCGTTGCTTTCTTTAAAACTATTCTAAATCAGCCTTTGAGTTTA
ACAAGTGTTGCATGAGGCATTTGCAGTAACAGGCTACATGGTTTGCATCCTATAACATCAAGCTTTCCGC
ATAGAAGCTAGACTAAGAGACATTCAGACTGATGCAAATTTGACAGTTTAGGCCTAAAACTGGCAATCTT
TTAAGCTGCAGATAAATGAAAGAGCAAGGGATAGCATGAGTGCTGCATGGGGCTCAGATTTCAGATGTCT
TCCTTTTTTTAACCCATACTCAAGCTTGCAGAATTCACAAATATATAACCTCATAATTCATCGACTTCAA
GATTTCTTACTACTCTATTCACATAGACTTTTCTAAAACCAATAAGGGGTTAGGGAGTAAGACATCTGCA
AATAAAAGCAAAATATTTACACAAGGTTGATGTTTAAGCATGAATAACAAAATCATTCTTTTGCTCTAAA
GAGTGTTTGGAAATACACATTTGGTTCATTTCCATTCACAGTTTTCTAATGAACATACAAGTTCTGCTTT
CATTCATTTTCACCAGCTAGCAGGCTTTTCATGAAAATGTTATTCAATCACAAACATTAAACTAATATTG
TTGGCATTCTGCATGACATTTTTATTTTCCAGGACAAGCTCATGATATTTTTGCCGGTAAAATAGCTGTT
GAGTAGTATATTTAAATTCCCCCTTCTGATTTTGTTTGTAGGCCTGAAGACCATTGTGGGGGCCCTGATC
CAGTCAGTGAAGAAGCTTTCTGATGTCATGATCTTGACTGTGTTCTGTCTAAGCGTGTTTGCGCTAATAG
GATTGCAGTTGTTCATGGGCAACCTACGAAATAAATGTTTGCAATGGCCTCCAGATAATTCTTCCTTTGA
AATAAATATCACTTCCTTCTTTAACAATTCATTGGATGGGAATGGTACTACTTTCAATAGGACAGTGAGC
ATATTTAACTGGGATGAATATATTGAGGATAAAAGTAAGATATACTCTATAAACCATTAAGTTGTTTAGT
TCTCTAAATATTAAATATTATATATAATGGAAATTATCTCAATTTAGATGTGAATCAAGTGACTTAGACT
AATTTAAGATGATTTAATACATATAAAAGAGATATCAAATGATACCTTATTCTATTTTTCTTATCTGTCC
ATTGATATAGTAAAAGTTCTCATTTGAAAATGTGTTGTCTTATACTCATGTTGAAAGTAATTTCATATTA
TGCCATATTAAAAAATGTTTATTTGGTAGACATTAATCAGGTTTTTCAGTCATTTTAATAAATAAGTCAG
TAGTTTGAACTATTCAGTGTATTCCACTGAAATGTGTTAAGAAGACTGAGGGGAAATAATTTGGCCCTAT
TTGGTTGATGCAACATATGTATTGAGTACATATGCTATATCTGAAAATAGAGAAACCATTTATCAAGATG
AAATAAGAATTTGTGTGCTCCTCAGAAGGTTAAGTAACCCTGATTTAGCCATTCACTTATTCATATTCTA
ATTAGTCCCTTTAGTGTCATCATTGTATTGTAGTTACCAGTTTAGTTTGATTATATTTAAGGTATGAACA
TCAGAATAAGCTTATGCCATATACTTCAGCATGATTTCTTAACATTGAGCCCAGCCCCTCTGTCATTTTT
CATATGTGTGTGCATGTTTGTATGTGAATATAAAAATACGTATGTTTGCATGTGTGTGCATGTTTTCTGA
GATCATCTTTGCAACTTACTGAAGTTATATGTCATGCCTTAAAAATAAAAACTAGATAGCTCTCCATAGC
TTAAAAATAAAAACTAGATATACTCAGACAACATATCTCTCCAAAGAAACAAGTTTATTTTCTTCATTTG
AAAGGCAGAAATCAAGCAAAAATTTCAAACAAAACACTTATTTACAGTATCATAAGAGGGAATAAATACC
TAATCCCACTTCTCACAGGAAATTAAGTTAAAATTGGCGGGAAAAAATGTCTGAATCTATTTTGAGCCTG
GGGAGAAAAGTATATGTAAGGTAAAATTTATTTGCATGAAAACACCTAGAAACAACAAGGCTTTCTTCTT
TCTTACTTTTTGTGCCCAGCAATAGACTGGCAGCTCTTTCTTAATGTATCCCATGCAATTTGAGCTTATA
TTTGCAATGAATGCTGATATAAGAATGTTATCATAGTAATTCCTTCTGAACATTTTTCTTTTTAACATAG
ATTTGCTAACCATTTGTATAATCAAAAATGTTATATATTGATATTTGTTCAATATTGTGAAAAATCTCTT
TAGCCATATATATTTATTAGTTTATCCATCTCATTATGATTGAAAACATTTGTGAGCTTTGCCACCTAAA
CAGGGTGGCTGAAGTGTTTTACAGGATTTTAATGATTCTTTCTATTCCTTTCTCTTTAAATAGGTCACTT
TTATTTTTTAGAGGGGCAAAATGATGCTCTGCTTTGTGGCAACAGCTCAGATGCAGGGTAAGTGATGCTT
CCTACTGAGTTTCAGTCCACACTGCTCCATCAGTGTCAATAACCTGCCACCTCCCACTCATCCAGTCCCA
CTCACTCCTCACTCAAAACCCTCCATAAATTCTACTTCACGGTGACTCTCAGAATAGCCAGGATAAGTGT
AGATTCTCACCTCTTTCACACAGTCATTTACTGCAATTATTTTTCTATGCTAGGTCACATCTAATCTTCC
AAATTAGTTCAATGTAAAATAGAGAATAAAGCAGTATAATATGCATCTGAAGCTTAATAGAATTCTTAAG
CACATACTTTTTATAAGTGTCATATTTTATATATACTAATGTGTTCTCCATAGCTTAAAATGTAAGATCT
CTGAAAATAATGTTAATATCTGAGACATGGGGAGTATTTAGCATATTTTAGCAAAGTGGTTACAAACATA
AACTGGAGAGTCTGCATAGAGTCAGACTTTGACTCCATCATATAATCTCATTTCTTCTTGCCTTCGTTTC
CTTATATGACAAATGGGTATAATAATAGGGTTTTTGTGATGATGAAGTGGATTAATAAATGTATAGCTAT
TTAGATCGCTCAATAAGTGCTTGTAATTGTTATTATTGGGATCATGCAAATGTTTGCTATTAAGAAACAT
GGAGCTAAATCCTAGGAAAATTTAAAAACACAGTTAATTTTCTTTATTTAGCAAGATTTTAGAGCCACAC
ACAAAAGTCTAATGCACTTTCTTTGGACGATGATACTGTGGACATTAGTAGCTAATACCTGTAGCAAAAT
TCCCAGTGATAATAGGCTTTCCATTTGGCTCCTACGATCAGTGCTATGCTGCCTTTATCTTCAGATTCCA
ATGATAAGTAAATCAATTGATTTTCATTCCTTGTTTGTACTGTACTAAATGCGTTACATACAGTATCTTC
TTCAATGTTTGCAAATTTGTGAGACAGGTTCTCTTATTAGCCCATTCTCACATGCGAGGTGCCTGAAGAT
TAGCAAGTTAAGTAACTTGCCCAAGATCGTTCAGCTCAGAAGTGTCAGGCAAGACATTGAAGCCAGGTCT
GCTTGATCTTCAAGGTCCTCCTATGACATTTTTACCACACAGTGTCATTCACTCCTTGCAGCATGCCCCA
CCTATCCTTTTCTCACTTCTTTACCCTGTTCCCACACTTACACACATTTCTGCCTCAAGACATCCTCAGT
GAAAATCAACTTTTTCCTTACAGACTTTTTTAACTGCCCTTAAGTCCCAGAAGATATTAATCATGATATG
ATTGCTTTTATATGGAGACATAATAAATATAATAATGACAATTATGAATCACAGAGGAATCCACAAAGTA
GACCTTATAGATTCTGTTATTATATAAATCAGTCCACTTAGTGCTGAGTTAAGTACTGGGTAAGGTGAGA
GAAATCGGCTTTTTTCTAGTGCCTGTATAAAACAGACATTGGCATATATTAAAACAGGAAAACCAATTAG
CAGACTTGCCGTTATTGACTTCCTTTCTTTCCTCTAACCTAATTATAGCCAGTGTCCTGAAGGATACATC
TGTGTGAAGGCTGGTAGAAACCCCAACTATGGCTACACGAGCTTTGACACCTTTAGTTGGGCCTTTTTGT
CCTTATTTCGTCTCATGACTCAAGACTTCTGGGAAAACCTTTATCAACTGGTGAGAACAGATAAAATCAT
TTTTCTGAGAATCATAAAACACCGAACTCAAGAGAATTGCTGTAGAATATTTTATTACTTAGAGTGTAAG
TTTGTAACATCCTATATAAAATTTATTAAAATCTCTCTTCCATTTTGCAGACACTACGTGCTGCTGGGAA
AACGTACATGATATTTTTTGTGCTGGTCATTTTCTTGGGCTCATTCTATCTAATAAATTTGATCTTGGCT
GTGGTGGCCATGGCCTATGAGGAACAGAATCAGGCCACATTGGAAGAGGCTGAACAGAAGGAAGCTGAAT
TTCAGCAGATGCTCGAACAGTTGAAAAAGCAACAAGAAGAAGCTCAGGTATAGTGAACAAGCATACGGTC
CTTTGTTTTTCTTTATCTAAATTCTTTAACCTAAATGTTGAGGTCAGTGGCAAGGTAGTTGACATTAGAA
ATAGGTCATATGTGTTTGGTAAGTGCTAGGAGCCTGTTTGGTTATTAAGAAGTTATTACTTTATTGCAAT
GATCTCTGTCAATAGTGTCAATAGTAATGGCATCAAAAAATGGATAATTATAATTGCTTTACTGACATTT
TTTTCTCCCTTGTGACTCCTTGAGGAAATTAATGATTAACAAAGGCCTCATGTACTCAAACTTGCAGAGT
AGATAAACCTACATGTCCTCAGTTGAAGTATTTTCTTAGGGGAAGAGGAATTCAGTTACACTTGCTTCTT
CATTGCAGTATCACCAGAGGTGGTAAGGGTCAGAAAACCAGAATCAAACTAAGAAAATTATTTCATTGAG
TCTGGAAAGGCAAAGGCTTATTCAATATTTGTTCTCTTTTATATAAAGTGTACAAATGCAAGTTTGTGGG
TTACATCAGTAAATCACTAGTGTGTAAACATATTAAAACATTAGCACTCTCTGCCTCCTACTCTACAAAT
CCTTTAATTTGGACTTGACAAGCCTTCAAAATAAGGCAAGAATTTCTCTAATTATATTTGCTTGACTTAA
TGGCATTAACTAATCCAATTGCCTATTTTTGTCTTTTCATGTATGGTGAATACAATTCCCTTTTATTACC
GAGTATTCCTAAATATGTAATAAAGGTCAAAGTATATTGCTGTAATAGCAACAAAACTACTGTTATACTT
TACAAGTTCATGCAGATGCCATGATCTAGGATTCTCAAATAAACACTCTGTATTATGTCTTTGCTGTGCA
TTTCTTAGTGAAATACCCAATTTAAATCACGGAGAAAAATGTCATTAAAATAAAATACTTGACTGAATTA
CATTTAATAATTCAGACTAGCACTAAATTTCTTTATTGTGTGAAAATGGAATCAAAGGCAAATGTCTACC
AGGTTTAAATAGGAAGTCTTTAATTCCCATATTATTTCCTTCTTAAAATATTGTTTGAATTATAGAACAT
GTTATTATGATCTTTAAGTGTCTTGCTCATATTATTAGATAATTAGATATCATAGTGTGAGGACAGAGCT
TGAAGGTTCTCATAAAAGTCGTATGTATCATCTTCCATATGAATGCCCATTTTACTCTTTGATTGGTCTA
ATAACAATGTACTGTTTTCTAAAACACAGAATAAAATGGAGAATTGTTTTTCAAGATTATCTTCATGATA
TTGAAGCTCAATTAAGCAGTAACATGATAATTACTTTTTAAGTTTATATGCAACTTCCACATACTTTGCG
CCCTTCTAGGCGGCAGCTGCAGCCGCATCTGCTGAATCAAGAGACTTCAGTGGTGCTGGTGGGATAGGAG
TTTTTTCAGAGAGTTCTTCAGTAGCATCTAAGTTGAGCTCCAAAAGTGAAAAAGAGCTGAAAAACAGAAG
AAAGAAAAAGAAACAGAAAGAACAGTCTGGAGAAGAAGAGAAAAATGACAGAGTCCGAAAATCGGAATCT
GAAGACAGCATAAGAAGAAAAGGTTTCCGTTTTTCCTTGGAAGGAAGTAGGCTGACATATGAAAAGAGAT
TTTCTTCTCCACACCAGGTAAAAATATTAAATTACATGAATTGTGTTCTCATAAATTTTTTAAAAAAATA
TGCCAGAATTTAATGGAGAGAAAACCGCCTTCCACCTGGATGGCACAATGCTTTCAGAGTAGTGATGATT
ATCAAGTGTTTTGGCTATCACTTCAGAGAATTTGTGAGTTTTGCAACTTTTTGGAATCCCAGGAAGGAAA
TTTTAGATCCCTCTGGGTTTGGAAAAATTTGCGGTTTTGAGGTTTTCTTAAAGACTGAAAAATCTTGGAG
AAATTTTCCACATCAGGAATTATCAGCAGATGGTTCCCATCTCTTCTTAACTATTGTGCGTGGATCTAGT
GAACTTTGGGTTTTCTGAGTGACAAATTCCCAGAAGTGGACCAGAGACTCTTTTAGGCCACCTGCGGGGT
TGTTCCCATAAGGTGCAAACATCACTTGCCAAGTGCATTCTTCATGCCTTTGTTTCAAAGGGGACTGAAA
CAAAATATCTCTAAAAGTAGCCAAAACTCTCAGATAGGCAGGTACTGAGGGAGATTTATGACACGAAATA
AAAAGTGGTGTTTAGTTGTACTTGATTATCTGTGTTTCATGTTAAACATGGGACTTGCATTTGAAGAATA
CTGTGATTTATAAACTGCAACAAATATTCACTGGATGCCTCTGCCTTTTGTACTCATGCAAGTTGTTGAA
ATTTTAAAATTTAGAATCTTAATTGTCTTTGAAATTACCAAGAGAATTCACAGGAATACACAGTACCTCA
GAAGACATTTTCACCAGGAGTGAAACCTTAATACCTATACAGTAACAATAACAATTACAACAACAACATT
GATAATGGCTAATATTTATACACTGTGTTGTTATTTGCAATATTAATGCATTAAGCCTTTCACTGCAACC
CTAGGTAAGTTCTAATTTAGTTAATATCCCCAATTTTTATATGTGAAGAGAAGCAGGGGGCTGCATTCTT
TGTTGAGTACCTATTTTATGCTTGGCATTCTTTCTACATTCTCATAATTAATGCTAACAGTTCTGTAGAA
TAGTATTATTTTCATTACTATAATGAAAAAGCTATTTTGGATCAGTAAAGTTAAATAGTTGCACAATGTC
ATATAGCAATAGAAAAAGTTATTATGATCTTTGAGTGTCTTGTTCATATTAGGGTTGTTGGTATTCAAAT
TTTGCCTGGTACTGAAAACCTTAAAGTTTCCACTTTATCAAGTTGCCTATGAAGAATGCCTTTAAAAACT
GATAAGGAAATTTACAATATAACTTTATTTAAAATACACAATGGCATTATACTTTCTCTTTTACCTTTTT
ATAATATAGACAAGCTCACATAACCTCACATGTGATATATATAAATTTTTTTAGGTCAGCCTTATTCATT
TCAAATCCAAATAACATCATAAGATTGCATACTTGGGGATTAATTCAAATTTAACATAGGATCTTTAAAT
ATCAAAATTTACTTGGTCTCTTTCATTTTGTTGTCACAATCATGATTCCATTAGTAGAAACATTAATCAA
TAAATAGGAATCCTTTAAAAGGCAAACCCCCTGTTTACAGTATTAGTCATTCTGAAAAGGAAGGAAGAAA
AAGAAAGGGAGGGAGGGAGGGGGGAGAGAGAGAGAGAGGAAAAAAGGGAGCAAAGGAGGGAGGAAGATAG
AGTATTTTTGCCTACATTTTTACCTAAGTTTGTCTGAATTTTTGCCTGAAGTTGTCTAAGTTTTGGCCAA
AATTTGCCTAAATTTTGGCCTAGATTTGAAACTTCATATCAACTTCATATCAAACACTTACCACAGAGAT
TCTCTTCAATTTGCCTTATTTCTAATTGAATAAAACTAATTCTAGGCAAAATAGTGAAGCCTGATAAGGC
TAGGCTCTGTCCTTCCTTTTTCTGTACATTTTGTTCATAGATATGATATTCTCCCAGCAGCCTCTTCTTC
ATACCTCTACATACCTCCATTTCCCAGCTAGTTGGGTATTATAAGCAATCACTGACTTAGAAGACATGGC
ATGGCTGGCTCATATTGATACTTGTTTCTTAAGCAGTCTCTATATAAAAATAGAGTTAAAGACTTTATTT
TGCTTGATAAAGAAATAGTCAAACAAATGTCTAAGAGGATGGAGAGGGAGACAGAAAAAGACAGAGGGAA
AGGGAAAGAAAGAGAAAGAGAGAGGGGGGAAAGGAGAAAAGGAAGAAGAGAGGAGAGACAGAAAACCCTG
AAATCACACCAACCCCACTTGGCAAGCCCTGAAAGTAATACTGAAAATGTCAAACCAGATAGAAGTACTT
AATCTTGGTATAGCAATGGAGGGCCCATCGTGTCTGTTAGATTTTTAAGAGTTTGAGACCCCAAAATATT
AGGAATTATTGTTTTGTGATGACATGATTGTGTTGGTAACCATCTCTGTGTTTGTATTGAAAAACTATAC
AATGCAAGCCTTTACTGCAAGATTATAATTTCTTTAGTAGAGTAAGTGGAAATATGAATTGTTTCTCAGA
CTCTGATTTGACTTGTTAGTGTGGTAAAGAGGGGAGAAGAAAGTCAAGAATGTAATCTCTAAACAAGTTT
CAAGATAATCTGGATTTTTTTGAAACCTTTATAAGGTACAATTGACCTTAAATCATTACTTTATTATTTA
TTTGTGATAAGCTAGGAGTTTAGGAGTTTTGCTTTTTAAAGATTGGTTTGGTATGGGGAATATTTCTTAC
TGGCCATCTTTTTTGTGTGTTACAGCATTTGATTACTATGCATTTATGTAATGAATGTCAGCAAAAGAAG
TTGATGCTAACGGATGGGGCACAATCATTTCTCATATAGCTGTCACATGTAAACTACGTTTTTGTATAGC
TTAATTCATCCATGATCCTTGAGAAACATGCAAACTTATAACTTATTTTCTTCCAACCCTTCTATGGCTC
CAGCTGAATGGGGTACTGGCAGTTAAAATATAAACTCTTACTAAAAGCGATAGAAACATTCTTCATTGCA
AAGCATGTATTGTTTGCCTTTCTTTTTTAGCTAATGAGGAGCAGTATGTCACACATCCTGCAAATCCCGT
AACTGTTATTTCCTCATAGCTAATTCGAAGTCCCTTGTTAGAGGAGAGAAAGGAGACACGAAAAAGGATG
GATAGTCTAAGAAAGGCTTTAAAAAATAACTACTTGTATGGAAAATGATAAAAGAAAAGAATGAATGTTA
CTAATGTAGTTAATAGGATTAAAAAGCATGGGAACAACAAGAGGAGAGATGACTTCTGTTGTGGGAGCAG
TAAGTCTTCTTAGAAGTAGTTCTAGGCCGGGTGTGGTGGCTCATGCCTGTAATCCCAGCATTTTGGGAGG
CCGAGACGGGCAGATCATGAGATCAGGAGATGAGACCATCCTGGCTAACACGGTGAAACCCCATCTCTAC
TAAAAAATACAAAAAATTAGCCAGGCGTGGTGGCAGGTGCCTGTAGTCCCAGCTGCTCGGGAGGCTGAGG
CAGGAGAATGGTGTGAACCTGGGAGGTGGAGCTTGCAGTGAGCCGAGATCACGCCACTGCACTCCAGCCT
GGGTGACAGAGTGAGACTCCGTCTCAAAAAATAAATAAATAAAAAAAAAGAAGTGGTTCTTACTGTAAAT
AATGAATAGAATCACATAAGATAGTGTTTAACATTTACAGACATTTAATAGAAACTAACAGATATTATTG
AGAAAAAGTAATTCTTTAGCTGGAAAGAAAATAAAAAGCATACTTATTGGTCAGTGTATTACTCTGTTTT
CATGCTGCTGATAAAGTTATACCCAAGACTGGGCAATTTGGCAAAGGAAGAGGTTTAATTGGACTTACAG
TTCCATGTGGCTGGCAAAGCCTCACAATCATGGCAGAAAGCAAAGAGGAGCAAGCCACACCTTACATGGA
TGGTGGCAAGCAAAGAGAGAGTGAAAGCCAAGCAAAAGAAGTTTCTCCCCATATAACCACCAGATCTCAT
GAGACCCATTCAGTACCATGAGAACAGTATGGGGAAAACCCCTACCATGATTTAACTATCTCTGACCAGG
TCCCTCCCACAACAGTGGGAATTATGGGAAATATAACTCAAGATGAGATCTGGGTGGGGACACAGAGAAA
TCATGTCATTCCACCCCCGGCCCCTCCCAAATCTCATGTCCTCACATTTCAAAATGAATCATGCCTTCCC
AACAGTCCCCCAAAGTCTTAACTCATTTCAGCATTAATTCAAAAGTCCACAGTCCAAACAAAGCCTCATC
GGAGACAAGGCAAGTCCTTTCCATCTATGAGCCTGAAAAATCAAAAGCAAGTTAGTTACTTCCTAGATAC
AATAGGGATACAGACATTAGCTAAATACAGCCATTCCAAATGGGAGAAATTGGCCAAAACAAAGGGGCTA
CATGCCCCATGCAAGTTCAAAATCCAGCAGGGCAGTCAAATCTTAAAGCTCAAAAATGATCTCCTTTGAC
TCCATGTCTCACATCCAGGTTACGCTGATGCAAGAGGTGGGTTCCCTTGGTCTTCAGCAGCTCCACCCCT
GTCACTTTGCAGGGTACAGCTTCCCTCCTGACTACTTCCATGGGCTGGCATTGAATGTCTGTGGCTTTTC
CAGGTACACGGTTCAAGATGTTGGTGGATCTATTATTCTTGGGTCTGGAGGACAGTGGCTCTCTTCTCAC
AGCTCCAGCAGGCAGTGCCCCAGTAGGGACCCTGTGTTGGGGCTCTGACCCCACATTTCCCTTCCTCACT
GCCTTGGCAGAGGTTCTCCATGAGAGCCCTGCCCCTGCAGCAAACTTCTGCCTGTACATCCAGGTGTTTC
TATACATCCTCTGAAATCTAGGCAGATGTTCCCAAATCCCAGTTATTGACTTCTGTGCACTGACAGGCTC
AACACCATGTGGAAGCTGCCAAGGCTTGAGGCTTGCACCCTCTGAAGCCATGGGCCTAGCTCTACATTTG
CCCCTTTCAGCCATGGCTGGAGCCGCAGAGATACAGGGCACCAAGTTCCTAGGCTGCACACAGCGTGGGA
CTCTGGACCCGGCACATGAAACCACTTTTTCCTCCTAGGCCTCCGAGCCTGTGACGGGAGGGGCTGCTGC
AAAGATCTCTGACATGCCCTGGAGACATTTTCTCCATTGTCTTGGGGATTAACATTCAGCTCCTCATTAC
TTATGCAAATTTCTACAGCCTGCTTGAATTTCTCTTCAGGAAATGGGATTTTCTTTTCTGTCACATTGTC
AGGCTGCAAATTTTCCAAACTTTTATGTTCTGCTTCCCTTATGAAACTGAATACCTTTAGCAGTACCTAA
GTCACCACTTGAATGCTTTGCTGCTTAGAAATTTCTTCTGCCAGATACTCTAAATCATCTCTCCCAAGTT
CAAAGTTCCACAAATCTCTAGGACAGGGCCAAAATGCCACCAGTCTCTTTGCTAAAATATAACAAGAGTC
ACCTTTGCTCCATTTCCCAACAAGTTCCTCATCTCCATGAGAGACCACTTTAGCCTGGACCTTATTGTCC
ATATTGCCATCAGGCTTTTGGTCAAAGCCATTCAATAAGTCTCTAGAAAGTTCCAAACTTTCTCACATTT
TCCTGTGTTCTTCCGAGCCCTCCAAACTGTTCCATCCTCTGCCTGTTATCCAGTTCCAAAGCTGCTTCCA
CATTTTTGGGTATCTTTTCAGCAGCGTCCCACTCCTGGTATCAATTTACTGTATTAATCTGTTTTCATGC
TGCTGATAAAGACATACCTGAGACTGGGCAATTTACCAAAGAAAGACATTTAATTGGACTTACAGTTCCA
TGTGGCTGGGGAAGCCTCACAATCATGGCAGAAGGCAAAGAGGAGCAAGTCACATCTTACATGGATGGTG
TCAGGCAAAGAGAGAGTGAGAGCCAAGTGAAAGGGATTTCTCCCCATAAAATCATTAGATCTCATGAGAC
TTATTCACTACCATGAGAACAGTATGGAGAAAACTGCCACTATGATTCAACTATTTCCCACCATGTCCCT
CCCCCAACAATGGAAATTATGGGAGATACAACTCAAGATGAGATCTGGGTGGGGACACAGCCAAACCATA
TTAGTCAGGCATATAAAAACCTAGATATTAGTAGGTATAAAATAATGGTTTTAGTTAGTTTTGAACCTTT
GGAGAGGAAAAGATCAAACCAATAATACTTAAATGATGAAGTTAGTATTCTTTCCAATAATAATTACATA
AATCCAATGCAAGCTGGCTGAAGTAAAAAAGAGAATGTATTAGATGATATAAATTATGTCTGTGAGTAGC
TTTAGGCATGGATGGATCCAGAAGCTCAAGACATGTTACCATGACCATGTCTTTCTCCATATCTGAATCT
AGGTTCCTCCCACATGGTGGCAAGATGTCACTGCAGCTCCAAACTTAAAGCCAATATTTTTAGCAGCCTC
AAGTGAAAAAGCACCATTTCCTCAACAGTTGTAAAAGACAGGGATATCTTTCAATTGACCAAGCAGGATT
TCATGACCATCTTTAAATTACGTATCGAATACACTGATTGGCTAGGCCTAGGACAGTGCCTGGAACACTG
GGAGACCCGAGCCTCAAGTCCTGCTGCATCCAGGAGTTGAAATTAGTGTCTCCCAGATAACCTGGTCTGA
GAGTTGAGGATTATTTCTCAAGGCTGAACAGGGTACTGATTTAAAAAAACAAAAAATTAAATGGTTGTGA
TCAGCCTCTTAGTGAAAATTAAGTTTTTGTAAATATTGCCCTCAGATTTCTTGAGACAGAGACAAAGGGG
TGAAAATTGGGGAATAAATCATACAGTTATTTCAGCTTGATTTAATTTATTCATGAAGACCATCATAAAA
TATGCAAAGGGAAGTGGAGAAGCTGCCCCGTGTACTATAATTAAACATCCCTACTAGCAAGATTAATTAT
ATTTCCTCCATGGTAAGATTTGCATCAGGGTGTGGTCACTAGCGAGCTCTTACTGGCTACATTTTTGACC
TCAGAGGATCTAAAGGTAGATTTGTGTTTAATTGTTTTCCATTGGGTTGTTAACTGAAATTAACTTCTAA
AGAAGGGTCTATCAACAGTATCAGTTCTAGATGCCCGTAACAGGACAAAACATTATGGGGACACTTCTGA
CTATGTTGAGGTGTGGGTAAAGTAGGAGAAAAGAGAGCAGAAGATGGAAAATGGAGGAAGGAGAAAAAGC
GAGAGTGAAATAGAAAAGGTGAACCTTGTAGAAAGTGCCAAAATGCCACCAGCAGTCATCAGAGGGGTGC
TTTCTTCCACATGTCCAATGACTTATCCTTGAGTAAGTCAATGACTATGACACAATGAATCAAATTCTGT
TTTTCAGAATGCCAGCTCTTAACTCTCTTCATCTCATTTTTGTTTCTTCTCTTGTTATTCATAGTCCTTA
CTGAGCATCCGTGGCTCCCTTTTCTCTCCAAGACGCAACAGTAGGGCGAGCCTTTTCAGCTTCAGAGGTC
GAGCAAAGGACATTGGCTCTGAGAATGACTTTGCTGATGATGAGCACAGCACCTTTGAGGACAATGACAG
CCGAAGAGACTCTCTGTTCGTGCCGCACAGACATGGAGAACGGCGCCACAGCAATGTCAGCCAGGCCAGC
CGTGCCTCCAGGGTGCTCCCCATCCTGCCCATGAATGGGAAGATGCATAGCGCTGTGGACTGCAATGGTG
TGGTCTCCCTGGTCGGGGGCCCTTCTACCCTCACATCTGCTGGGCAGCTCCTACCAGAGGTGAGGCCAAT
TAAAATTGCAGCTGATGTGAAGAGAGTTGTGACTGGTGCAGGCAGGAGTGTTTTTCCATTTCCACATCTA
AGAATTTGTTGAGTTTGTTGCCCAAAGGCTGGGAGTTTGTTCAATCAAGCTGTTAACTGTCTTGTGAAAC
TGTTCTATTCAGACTTTTCTACAAAGTAATTAAAAACCTAGGTTGGCTGTCAGAGAATATAATTAGAAGT
AATCTTTCATCATTATTACTATGGTATGAAACTCGCCAAAAAGCAAAGCAACAATTTATCAAGCATAATG
TTTGATTAATATAGTTAAATTAAATCCAAGGAAATTAATGCTCACTAATTAAATAAATACTTAAGGATTT
TGTGATTGTTGTTCATTTAAAAGGAGATTTGAATACTTCCACTTGCAGTAGATACTATTACTAAATAGAT
TTAAATCCCATAGTACAACATTGCCTCTCTTTGCAGGTCAGAGTGTTGTAACCTTTTTAGCATCCACTCT
AATGATCTCAACCATTGTAAATTTATACATGAAGAGCCATTCAAAAAGTACCTGGTTTGGAATCATGGGC
TGTCATTTTTAGAGCAGATTCCAATTTTTATATTACTGTCATAAACTCTTATTGTAAACAAAGTGGCCCA
AAACCAATCACATGGAAAAGGATTTCAGCTACATACTAGACACTTACAGGGCTATATTATTGAAATTTAC
TTCATAAACCATAAGAAGCTTTTAATGTTGGTATTAAATAAAATTCCATTAGCTATCAAGACATATTTTG
GCAATGTCACTTGATTGTATTTTATAGCATTCAAAATGTCTTCTTATGATTTTTTTTTCACATAGCTCCA
TTTATTATCATTGAACAAGCTCTTTGAGCCACATTAAAATGATACGGAGTTCGTTTTCAGTTACCTAATG
GAGGAGCTTCTTATCTTGGATTATAAAATAGCCATTATCTTCTTCACATTTTTTGCATGGCCTCTCCCCA
CCTCCTTCTACCAGAGAAGTGTCCAGGTATCCTGCAGTCAGGTTGAACCATGAGAAAAGTAGAACTTTAT
AGTGGAGGAACCAGGAAGAATGAAGAGAGGACATCAGCTCTTCTTAAAAAATGATCATAGATAACCATGT
ACCAGACACTGTCCAGAACACTGTACATGTGTTAAGAATCATTTAAGCATCATAACAACCCTTTTTGATA
GTTAGTAACATTATCCCAATTTTACAGAGGAGGAAACTGAGGCTTGTGCGTGGTGGAGCTAAGATTTGAC
CCCAGGTATGCTGGTTACTGAACCTACATTCTATCAACAGTGAAATATTGCCTCCCACTGAGTTATTTTT
AATTTCTTTAAATCAAAAAGAAGAGATGGTTAAGGAAATAAACACATAAACACTTTCATTTTAACCAATG
TTTCTCAAAATATACTTCACTTTCATACTACTTACATCAGAATCTCATGGGAGGCTTCTTAAAACTAGAG
ATTCCTGTGCCTACCCAGACCTTTTCAGACCAAACCACTGAGGAGAACAAGGTCTGGGAATCTTCTTCCT
TAACAAGCATCCCACATAATTCTTATACATAATAAAGTATGTTTATCACTGATCTTGATAAATGTTAATT
GGGTAAACAAAAGCAAATCTACAATTACCATGCAGGAATACAGACAGACTGTCAGTCTGTCAGAAATTAT
TTAGCATTTATCAATAATTATCATAAATCTCCTGTCCTATCAGAGATGATGGGACAAATCGCTGAAGGCA
AAGTTGGGGCCAGCTTGAAGCAAAGCTTTGTGTGGTCCCTTTATTTCCTGCTTCCTCAACTTCATTCTTT
AATCTTACAATCTTAAGTGCTTTGAGGCAGGGCACTGTACTATTGCAAAGTTGAGCTGAAGGTGCAAACA
AATGAAGTAGGCTTTTGGAGAATGCAGAGGTGAAATGACAATAGAAAATAAATAGCTATGGGCAAATGAC
ACCCTTGAAAGCACATCATTTCCTGTACTTTACACATAAATTCAATCGAGTAATGTCATTAGCAGTTTTG
GAATCTATTTGAAAATTAGACAAATCTAGGTTTGTACATGTGCTTCTGTGTAGAACAGAAGGGACTAGAT
GATCTTCATGTGACTATTTTTTTTTTCCTTAAAACTTTGCCTCTTTCTGACAAGCTGAAATATTTTAAAT
TCTAAGAGGCACCCTTTGGATTAAAAGACTTTTATTTTTAGAGATAGGTTATTTCTTTTCTTACTAATTT
TACTTGTTTTTTTACCTTAAAATTAATTTTAGAATGACCTATATGAATAGTTATCACCATTAGTGACAAT
CATATGCAATGAGTGGAAATTTTGGTTTTGAATATGTATGCATTAAAATAATTCAACTTACAAAAGATAA
AATACTGCTAATTGTTCACATCATAATAGGATGTGACCAAAATAAATAATATTTTGATCATATTATTATA
TTTTGATCATATTGTTCTTTTAGAAATAGGGAAAGTCCTCAAAGGAATGAAACTTTTTAATTTATTATTA
ACACTCAGACCTGCATTTGAAATTCTTTAGCTTTACCTTTTTTTTTCCTGTGATAATTGACATCATTGTT
TGATCTCCTGAAGTAGGAATAAATTTCCACCCATGTTGAAATCCTGATGAGTTTATTCTGGAGTAGGAGA
TTATCAGATCATTGTATCATTACTAAAAATCACAGTCCCCCACATTGGTATTATCTCCTTAAATTATAGT
CTCAGTGCCAAGGGTGGATTGTTTTGTGGATTAAGTTGTTTTTCAAATATAGGACAAAGTTATAGACTAG
TTCTAAAATTTAGTTTTGTAATTAGGAATGTTGGGAAATATTACCTGTGTCTAATGAATGAAGGCATTTT
GCAACTGGAATTCACATTTTAGGGGAACTGTTACTGATGCATATGAAGGAGACTTTCAAACCTTTTTGTT
CATATATTAAACTACCTGAATATATGTCTATAAAGATCTAAAAACTCAACCTGGGTGAAAATTAAGAAAC
AATATGTTTTGGTCTGAAGTCCTAAGTGGGATTGGCTGAAATGCTAAAAGGTTATCTGTCCAGTAGTGGA
CCTGGTCCCTCCAGCCCAAATCCCTGGGATAGAGGCATAGGAAAGCCCACCTTGACAAACCCAGGGCTCC
CCAAAAGCTGAAAATCTGACAGACTTTTAAACAACCCCCAAAGAATTATCATTCCAACAATATCTTAGTG
AGCTTTTTACATCTGAGAAAGCATGGTGTATATTTAGTTAAATAACACCTGTTGTAGGAATGCTTTGGGC
TTTGCTGCTTTCAAAAATAGTGGTTATTTCATCTGAAATTCTACTTCTAGGGCACAACTACTGAAACAGA
AATAAGAAAGAGACGGTCCAGTTCTTATCATGTTTCCATGGATTTATTGGAAGATCCTACATCAAGGCAA
AGAGCAATGAGTATAGCCAGTATTTTGACCAACACCATGGAAGGTATGTTAAAAGTCCTGCGTCACAGTT
ACTTGGTGCTTTGGTAATGATGAAAAAACACTTCATAAATTTCAATAAAATACTTCCTGACTTGATATTG
TATCATTATTACACATTTTACTAAATAACAGTAAAATCCGTGCATAACTCATGGATTCTATTATCTTCCA
CAGATTTTTTTTTTTTATATTTAGCCTCCAGAAAGCTGCTGCAAATGTAAGGTATATTTTGAACACCACT
TTCATACATTAAATTCTAAACATTGAAACTTGTGTGCATGACGTTGAAAAGAGTGTAATGATAAATGCTT
ATACTTATGATGATGCTAAGCCATTTGGATTATATTAACTGCTTGAGACACAAGTTATAAAATCCTATGA
CTTAACCAGAAATATAAATTAAAAATGTGAATTAGGGTTTGATATTAACTTCCTTGAAGCAAAGTGTTTA
AAATTTTGTAGTCCTACTTTTGCCTTTCTCTGACCAGATTCTTACAATATATCAGCTTTCTCTTTAGTTG
CAGATTTTATCTGAATAGTTAACATAATGTGTAGCAGTCTGGATCTCAGAATGCCAAAATAAAGACTTTG
GGGACAGCTTAATCTGTGATCAATTTCTGGCTCTGCCATATGTTAAATGTGTTAATTTGTGACTTTGAAT
TTCAGTCTCCTCATCAGTAAAATGTGGATGATGATGTTTAGGCATAAGGTTGTTGAATGGATTAAATAAG
CCTTCTTAGATAAAACACTGATGTATTTGGCATGCAGAAGACAGTTAATAAATATTATCAATATTAGTTG
TTTTGTTGTTGTTATTTTTGTTAATTCACATGTTTTTGCCTTTCCATACTGTAAGTGAATTCAAACAACT
GTCAACTTCAACTACTTGGAAAATATTTTCATGTAAAATGTATTCTATCCCCCTTCCTTGCCCTCCTATT
CCCTCCTCTCCCTATCTCTTTACAAACCTTCTCCCTTGTACCCCTTCCCAGGTATGTGTGTGAGTGTGAG
TGTGTGTAGATGTGTCAAGGGAGAAGAGAAAAGGAGAATGAAAGCAAAAGAGAGCAAGCATACACGTCCC
TTTCTTATTGATAATTAGATTTTCTCTTGAGATTGGATAGATTCCTGGAATAATTCTTTTCCTGTCTGTA
TGCAAAGATCCCATAATATTATTAATACCAATACGAAAAGCCTGAAAATCACAGCCAGAAAAAATTCACA
GTGTAGACGACTGTGTACATCACAGACAAGTCAGTATTACAAAACCCAATTTTCATAGTGTCCTATTTCA
GTATCCTAATGCAATTCACTGATTTCAATTGAATATTAAACTCTAGTACGTTCTTCCCCAACCTCGCCTG
CGTTAGCTTGCACTCCCTCTTCCCCCCAGCTGCCAGTAGCTTGCTCCTCCCTGTCCCTCCAGGTAAATCT
TTTGAAGATTGTCTGGCCTTCCGCTCCTTGCCATAGCAAAACCACTGAGAGGAAGCTGCCAGTGGTTCTG
CTACCGATGTCAGCAGCATGTCTGCTCCCTAAAGCAGGAAGTAGAGAAGGAGACAGGGTAAGTCTAAATC
AACAGTCATGCTTTGCACTTCTGATAGCATTAAGTTTGAGCTAAATAAGACATTACTTAAAAAACCTCAA
ATATCCACAAGATTGGACTTGCCAACTAATTAAGATTTGGAGTTCAAAATAAATGCACCCACACCTTTCT
CCATGGAACTATGTGACATGGGGTTGCTTAGGATGGAAAGGATGTTCTAGGAATAAGTGCAATCTAGGAA
GCTGAAGACTGAGAGTGTTTTCGTTTTATTATCTGCAGAGCTTTTGACTTGTGTATTTGTGAGAAATAAT
GGCCAAGTTTTTATTCTGTTTTTAATCAGTTATCTAGAATGAAAACTGACTTTTCTTTATTCAATTGTAT
GTAGACACATTGAGTGTGACATTTGTCAAGGTTGGTTGTTAGCAATATCACATACATGCATACTCAAGCA
GACTTAAGATAGTCCTTTTTTTTTTTTTTTTTTTTTTTGGTTTCTGATAATGGTGCAAAGTTTTCCTGGT
TGACATAATCTCTTTTCTTGGGGATCCTTTCTTCTATGTCTGATTATTGTTTATTTCACCTTTCCTTTTT
ATGAACCAGGCTTGTTGATCCGGTTGGCAATTTTTGTTCTCCTTCTTTTTAACTACAGCCAAGTCTCCGT
TGTCCAGGGTAATGGATAGCCTCATGCTTAATGAAGCAGTAGTGGATAAGCAAAAATGAACCATTTGCGT
TTCAAATTTTTAAAAGTGCAAAATCACATAGAAATGTTTTCTGGTCACACCTTTGTGAAGGATGGTGGGA
GGGTGAGTTAGAAGCGCCTGAAGAATCAAGGTGAGCCAGCAAAAGACACAGATTTACTGTAAGTGATTCT
ATGTGGATAACCCATGCAGGAATAATGGAGATGTGGCTGCAGTTCTCTCCTGAATGCTTTGCTTTGTTTT
AAAGTGTGAGATTCCCCCCTTTTTTTTGGAATGAATAATTGAATGATTTTATTTTAGAACTTAAACAACT
TTGCTCCGGTTATTCCTACTGTCAAGATGAGCCACACCTGCTGAATTTCATTTTTTAAACTTTTCTCCAG
TTTATTTTTTTTCTATCCAGTTCCTGTTTGCTTCTCGTTATTTGCTAAATGACAGCTGGCATGGAAAGAA
AAAACCATATTATGGCAGTATACAGACAAAATTAAATTTTGTAGTTTCTTTTTTGTTATTTACTGTAAAT
AATAATACCTCCTTTTTACCTTCAATGTAAATATAAGGTTATTTGGGAGTTTAGAAGTATTTACAAAATA
ACTAGTGCATAATAACTATATTTTTCTCTGAATTATAAATCAAATATTATTTATTGTTTACAAAGTTATG
TATTAAGGGAAATGGAAACAAACTGGGCACTTGAAGAATTTCAATATCCAAAGAGACAATTGACAAATCT
ATTTTTAGTGGAAATTTTAAAAACAATAAGCAATAAATTAATTTACTTAGGAAAATAGTATTATAGAATT
AATTAGTGGCAAATGTTGATTAGTAGAAGAAACATTATCATCTGTGGTTGTGATTGTCCTTTTATATGCT
GGTTCCACCTTTACAAGGTTTAGTTCATAGCAAACTGTGCCAGATATGGACAGATGTTCCAGTTGCCACA
ATAGTATTAAAGTGACTGACTAGAGTCAACTATGCCATGGATTTAAAAAGAAGAAACCTTCCCTCTATTT
CAGTGCTAAGAGGTGGTGGCCACATTTTGGCAGAACAGGTAATAGGGTGTACAGCAATGATATTGACAGA
AAACAACAAATTCTGCATATTTTTCCACTCATAAGTTGATGAAGAGATTATCTTGCCAAAGGAAGATGGT
AGACAGTTTTTCACTTCCTAATTCCCCAAATTTCTTTGCCAATATTCACCAAATTTCAGTATTTTGGGTG
TGACCTTAAAGATCTGTGCATTTCTGTCTTCTCTCCCAATGTTTGGTTGAAATTCTTCTTGACATCATTG
TACATTTTCCTTGAATAAATGCATTTTAATATAAAATTTTATGTCATGTTTGATATGAGAGTTATATATT
TAAATACATTTAAATAAATGTTTACCATGAAAATGTATGAATTATATGTATGTTTCACCTAAAATCCTTT
GTATTTTTCCAGTAATAAATGAGTTCCACTTTGTGAAATGTTGATTTGTAACAACAGTGAGGACTCCAGT
TCCTTAGGCTGGGGTATTTTCTCTTCTTTTATGCCCTCTAGTTAAATGAGAAATGTAGAGAGATGGAACT
TTGTTGTGTCTAATATGCAAGCCTATAATCTAATAAAATTTAATTTGAGACTTTTAAACTGAGATTGGTG
ACACTGACAAAATTATCTAATTAGAAGATCACCAAAACATATCTAATCCAAGAAACTGACATTCAGTGTG
ACTGATTAAGGTTCTTAGGACATCTCCTGAGATATCTCTGATAACATATATACTTCTTGCTCTACCTGGA
ACATGGATGAGCTTTAAGTGTATGCAATGCAAGTTCTACCCATTAGTTTCTAGCAGCCTTGAAGATAAGT
ATCAGACAGTTTAGTGTTGCCAATAGAATCTTGGAAGCTATGTTTAGCCAGGATACATTTGGAAAGCTTA
CTAGCCTTTCTGTACTGATCCTTTCTATGACAGCAAACCCATTGTAAAATTTTCCCTGTTCCTCCAGCAG
ATTAACCCATAATATCTTTTAACAACTTTAGATTTTTTAAATTCCTTTTAATTTAAACCAAATCTGCTTA
ATAGAAAGTAAGCAGTTTTCATGAGGATTCTAACTTTTTTTCTTCCAGAACTTGAAGAATCCAGACAGAA
ATGCCCACCATGCTGGTATAAATTTGCTAATATGTGTTTGATTTGGGACTGTTGTAAACCATGGTTAAAG
GTGAAACACCTTGTCAACCTGGTTGTAATGGACCCATTTGTTGACCTGGCCATCACCATCTGCATTGTCT
TAAATACACTCTTCATGGCTATGGAGCACTATCCCATGACGGAGCAGTTCAGCAGTGTACTGTCTGTTGG
AAACCTGGTAAGCCTCACTGAGAGTTTCTCTTCCTCTTGAAAGAGTTTATAATTGCCTTAGTGAATTTTA
CATATTGCTCTCAAATTAAATATCAACTAATTGGCCATGTATATCTTGACATCAAATGTTTAGCATCCCT
TTTAAATAACAAAAAAATGTTGCTACCATAGTGCAAAAGAGTCAAAGAATTTATGTACAATTTGATTTAG
AATTGAATTTAAATTGCTTATTTATTAGAAGATGATTCTGAATTGTCCTCCAAGGACATTGATCTATAGC
AAAATTCTGACATATTTTTAAGAACCTTAGAATAGGTTCTTTAGGACATGTCTGTGTTTACTAAACAAAT
GATAAAATATGCCCAAGTCAGATAATTTTGAAATATCACTTGTAAGTACTTGAAGATGGACTATGTAGGG
AGGGGACATCATCTGGGGGATTATTATTTTTTGTTTTTGTTTCTTCTACAGTCTTAGCAATATTAAATTT
AGAAATTATTTATTTAATTTTGTTAAAATATATATTAGCATTTACTGGATACATATTGATATTATAATAT
AGTATACTATAGTGATAGATTTTAAAGTGGTCTTATACAGAGAGACAAAATGAAGAAAATCACGCAAAAA
AGTATATTTATACAAGTATAAAATGCTTATTAAGTTCCCAGCTTGATAAATGAACATATAAGCAGGTTAT
ATAGAGATTGTAAATAATACGGTCTAAAGTATAATATACAATATTTTTAGGCTCTGAAGTAACCACTATA
TTTTCGAATTATATTTCAGGTAGGACTTAACTGAATTAAATGATAAAGTTGGCATATGTTGGCCTCTTAT
TTTGTATCAGATATTGGACTAAATGGTTTACATTAAATCTCTCTCTCTAGATTCCCACAAAACCAGTCTT
AAGATATTTACTATAAATTGTCTGTATTTCGCAGTTGGGAAACTGTATCCATGAGAAATTAACAGAGAAA
TGGAAGGTCTGAATGCTGTATCAGACTCTGAAAGCTATTTCAGAGACTATCATAAGCTATGGGCAAAGAT
CACAGACGCTTAGAGTAGGAAAGGATAATAATTTTACCTAGTTCAAATTTAGAGCTATGTAAGAATTTCT
TCAACATTATTTCTAAAAAAAAGCAGGGTGGTGGGGTGGCAATTGAAACAAGAAAGCCTTTGGAGGTAAT
ATATTGTGATCCAAATTGAATGAGCTTAAGCAAAAATAAAGGGAATTCATTACCTCACTTTACTGAAGCC
CATTTGGAACTAGTTCTAGGTTCTAACCTGGCTAGATTCAGGTTCTCAGCCAGCATCATTGGGAATCAGC
TTCTTGTCTCTCTTCTTCCCAATATGTGACCTTCCTCTCTGGCTGGGCATTTTCCTACTATTGCCAAGAT
TCTATCAACTTTTATTCTACTAATGTAGCTCCATGTACAGAAAGTTTGTGCCTCCTAACTAGTAGCTTTA
ATTAAGGCCCACAAATTGAATATCATTGACCTGGTTGGAGTTACATGGCCATTTCTAAACCAGTCAGTTA
CCCTAGCTCTTGGTTTTAATGCCATATTCGCCTAAATCAGAATCATATCTTATCACTGGAGGCAGAGTAA
ATGAGTGAAATTATGAAGACAATAATTAGGATACCATTACCAGAAGGAGGATGGATTCTAGAAAGAAATA
AACAATAAATAGACAACTCAAGCTGGGGCTGGTCTTACGTGTTAAGGAATGTAGGATCTGTATCTAAGGT
GAATTATGGAATAGTAGAAAATCCATAGGCAGGATACACATTCTTCTCAAATGCCTATAGGATATTCCCT
AGGATAAGTTATAGGTTAGGTCATAAAACACACATCAATAAACTTAAAAAAATTAAAATAATACAGAATA
TTTTTCTGATAAAAAATAAAATGAAATTAGAAACCAATAACAAGTCAATGTGGAAAATCACAAATATTTG
GAAATTAAACAACTTGCTCTTAAATAACCAATGAGTCAATAAGATGTCATGAGAGAAATTAGAAAATACT
TTAGGATGACTGAAAATCAAAACAAACCAAACTGAAATTAATGAGGGCAGCTAAAACAATATTTAAAGAC
AAATTTATACTCTAAATGTCTATAGTAAAATAGGAGATTTCCCAAATTGCTAATTTAAGCTCCTTTTTAA
TAAACTAGATAAAGAATAGCAAATTAAACCTGAAGTTAACAGAAAAGACAAATATTAAAGAAGAGAAAAA
CAACAGAGAAAAATCAATAAAACCAAAAGTTATTTCTTTGAAAAAATCAACACAATTGACAAATCTTTAG
TTGGGCTAACCAAGAAAAAAAGAGGAAAGACACTAATTATTAGAAGTAGGAATGAAGAGAGGATATTACC
ATGGATCTTTTAGAAAGAAAAAGGGAGCATAAGAAATAAAATAAAATTAAACGCCAGCAAACTAGATAAC
CTATGTGAAATTGAAAAATTCCTAGGGAGTAACAAGCGCCTGAAACTGACTCAAGAAGTAATAGACTATC
TCAATATACTTATAATAAGTAAACATTGAATTCACAATTAAAAAAAAGAAAACTTCCTGCCAAAAAAAGC
CTGAGCCCAGATAGTATCACTGGTGAATTTTGCCAAATGTTCAATGGAGCGTTAACATCAATCCTTAACA
AACTGTTCCAACACATAGAAGAGAAGGGAATACTTCTCACCTCACTTTCTGAAGCTAGAATTACCCTGAT
ATTAATGCCAGACAAAAATAATGCAAGAAAAGAACACAGACACAAATATAGACCAGCCATATCCCATATG
AACATAGGCACAACAATCCTCAACAAAATACTAGAAGCCAAATCACATAACATATTTAGATTTTTAGATT
ATGTACTCTGAATAAGTGAAATTTATCCCAGTGATGCAGGGCTGGACCAGCATAAAAAATTAATGTAATA
TATCATATTAATTTTAAAAAACTATACAATCATCTCTATAGATGCTGTAATCACATGGAAAAAGCCAAAG
TGTTTCATGATAAAAACACTCAGCAAACTTGAAATAGAAAAGAACTTCAGCCTGATAAACACCATCTCAA
AAGACCCCACACCTATCATCATTCTTAATAGTTACTTTAGATGCTTTTATCTTCAGGTCAAGAAGAAGGC
AAGGATATTTGCTCTTGCTACTTCTTTTCAATATTGTACTGAAAGTTCTAACCAGGGAAATAAGACAAGA
AAAAGAAATTAATGGCATCTAGATTGGAACACAAGAAGTAAATTCTATTAACGAATACATAATCTTGTAT
TTAGAAAATCCTATAAAATACACACACACACACACACAGCTGTTAGAACTAATAAATAAGATTGCAGAAT
ACAAGATCAATATACAAAACTCAATTATATTTCTAAACACTACTAATGAACAATCAGAAAATAAAATTAG
GAAAATTCTATTTATAACAACATGAAAAATAATAAAATACTTAGGAATAAATTTAATAAAATAAGTGTGA
GATTTGTACACTCAAAGCTATAAAATATTGTTGAAAGAAATTAAAGAACTACTAAATAAATGAAAGCACA
TTTTATATTCATAGATTAGAGGAAAATATTGTTAAGATGGCAATACTCACCAAATTAATCTACAAATTTA
ACGCTATTCATATCAAAATCCCAGCTACCTATTTTGCAGAAAGTGATAAATTGACTGTAAATTTTATATG
AAAATGCAAGAGACTATATGCCACACAATCTTAACTAGAAAAAAAATAAAGTTGGAGAACTCAAACTTCC
AAATTTTAAAACTTACTACAAAGCAAAAGTAAGCAAGATAGTTTGGTACTGGCATAAGGATAGCTATATA
CATCAATGGAATAAAATTGAAATTCCAACAGTAAGTCTTCATATTTATGTTAAATTAATTTTCAACAAGA
CCACTAGACAATTTTATTAAGGAAAGAAGCCTTTTCAACAAATGGTGCTTGGACAAGTGAATATCCACAT
ATGAAAGAATGGAATTGAACCCTTACTTAATAACATATATAAAAATTAAGATAGGTCATAGGCCTAAAGG
TAGAGCTAAAACTATGAACTGTTAGAAGGAAATTTAGAAGTAAATCTTCATGACCTATTATTAAGCAATG
ATTTCTTAGATATGATACCAAAAGCACACACAATAGCAATAAGAAAAAAAAGGTTCATTGGACTTTATCA
AAATTAGAAACTTTCATACTGCAAACAATATCATCAAGTAAAAAGACAACTTAAAGAATGGAAGAAGACA
TTTGCAACCCAGATATCTGATCATGATTTGTATCTAATATATGTAAAGGATTATTATAACTCCACAACAA
ATAAAATAGATAACTCACTAAAAATGATCAAAATATTTGAATAGACATATTGAAAAAGGAGTTAGACAAA
AGGCCAATAAGCACATAAAAGATGGTCAGCGTCACTGGCTAATTTTAGGGGAAAGGCAAATCAAAACTAA
AATGAGATACCACTTCACACACACTAAGATGGCTATAATCAGAAAGAAAGCCAATACCATTTTTTATCAA
GGATGTGGAAAAATTAGAATGGTTATGCTTTACTTTGAGAATATAAAATGATGCAGTCACTTTGGATAAT
AATTTAGCTGTTCCCCAAAAAGTTTGGGGTAGAGTTACCACATGACCTGGCAGTTTTACTAATTTCTTCG
GATATATATATCTAAGAGAATTAAAAACATATTTCAACATGAATGCTTATAGAATATTATTCATAATACT
AAAAATTAAAAACAATTCAAATGTCTATCACTTGATGAATGGATAAACAATATCCATCCAATAAATGTTA
TTCATCCATAAAAAAGAATGAAGTATTGCTACAATCTACAACATGAATAAACCTTGAATATATTATATTA
AGTGAACAAAGCCAGTCACAAAATTTACATATCATATTGCTACATTTATATGAAATGTTCATAACAGGCA
AATCCATAGAGACAGAAAGGAGATGAGTGGTTGCCAGGCAGTAGGGATTAGGGTAAATGGGAGCAACAAC
TAACTAATGGGTATGCAGCTTCTTTTTAAGGCGATGAAAATGTTCTAAAATTCAATAATGGTGATGATTT
TACAAGTCTGTGAATATATTACAAGCCACTGAATTGCATACTAATATTTCATAGTATGCATGTATCACTA
TTTTTTCTTCCAATCACCTATTGATGGATGTTCAAGTTGATTCCAGATACTTTACCCCCATGTTGCAGTA
AATGTTCTTGTATATATCACCTTTCATAATGCTGTATTAACTTCTAGAAGATAGATTTCCAGGATTGAAG
TATTCCCAATATTAAACATATTCATGTCACATAAAAATTAGATGATATGCAACGAAATAGGGAAATGTGA
CTCATAGTTAATAGATTAAAGCGGTACATAGAAATAGATCTGGACAGGAACAAATTGTTAGAATAAACAA
AAAAGGACTTTGGAGGCACTATTGTAAATATGTTGAGCAATTTAAAGGAAAAAAAGGGGTCATAAGGAAT
AAACAGAGAACATGAGCAGAAAAAAATGAAAACTGTAAAAAGGAATAACTCAGACATTTTATAATTGAAA
AGTACACATTCTAATATGAAAAATTCACTGGATGTGTTTAAAACAAAATTGCAGATGTCAGAAGAAATAG
TTGTTAACTTGGAGCACATTAATAGAAATTATCCAATTTGAACACTAGAGAATTAAAAGGAAAAATGAAC
TGAGTCTAACTATAATATGGGATAATATCAAGTAGTCTAAATATTTATATAATGGGAGAATCAGAAGGAG
AAAATAGAAAGAATGGAACAGAGCAAATATTTGAAGAAACAATGGCCAAAAATTTCCCAAAGTTGGTGAA
AAATAGTAACTTACAGACCTTAATGTTACCTTATAAAATGTTAGTGAACATCAACAGGATAAATATAAAG
TAAGCCATACTTAGGCACATCTTAGTCATGCTGCTGAAAACAACAATAACAAAAAGCTTTGGAAACAACC
GGAAGAAAAATATTTACTATATACATGAGACTAATGTCTTACCAGAAATAATTCAGGCCAGAAGAAAGTG
GCCAGATAAAATAAAGAAATAAAAGCCCATTATTCTACAGAAAGTGAAACTATCCATCAGAAATTAAAAT
GAAATAAAGAATTTTAAGATAAACAAAAACCTAAAAAAGCTGTTACCACCAGAACTACACTACAAGAAAT
GTTAATGGAAGTTCTTTAGGCCGAATAGGAAAAATATCAGATGGAAATTTGTTTCTGGGAATGATGGTCA
CTAGATATGAATAAATGTGGGTAAATACGAAAGACTATCTTTTTTTCCTTAATTTTTTTCACATTTATTT
TAGGTTCAGGGGTATGTAGACAGCTTTGTTGCATAAATAAATTGTAAATCACAGGGGTTTGGCATACAGA
TTATTTCATCATCCAGGTAATAAGCATAGTACTCTATAGATAGTTCTTCAATTCTCATCCTCCTTCCACC
CTCCACCCTCAAGTGTCTGTTGTTCCCTTCTTTGTGTTCCTGTGAACTCAATGTTTAGTTCCCACTTATA
AGTGAGAACATGCAGTATTTGGTTTTCTGTTGCTGTGGTAAATTTTTTAAAAAGACAACTGATTGTTTAA
AGTGGGGATTTATAACATAGGTAAGTGTAAAATTATGAAAAGATTTGAACTTTTTTTTTTTTTTTTGAGA
CGGAGTCTCGCTCTCTCGCCCAGGCTGGAGTGCAGTGGTGCGATCTCGGCTCACTGCAAGCTCCGCCTCC
CCGGTTCACGTTGTTCTGCTGCCTTAGCCTCCCGAGTAGCTGGGACTACATGCACCAGCCACCAAGCCTG
GCTAATTTTGTTGCATTTTTAGTAGAGACGGGGTTTCATCGTGTTAGCCAGGATGGTCTTGATCTCCTGA
CCTCATGATCCGCCCGTCTCGGCCTCCCAAAGTGCTGGGATTACAGGCATGAGCCACCACGCCGGGCCAG
AACATTTTATCTAAAGTGTTACAATATTAATTTTACATAGAAAATTACAAGAAAGTATCCCTCATGAACA
CAGAAGCAAAAAATCATTAGCAAAATATTATCAATAAAATCTAGCAATAGAGAAAAAAGTAATAAATACT
TCTTAAACATGTGGGGATTATCTCAGAAAAGTAAGATTCATTTAACATTTGAAAAGTGATCAATTAATTG
GCCATATTACCTCTAAAAGAATAAAGGAAAGCCTAAGATCATCTCAATAGATGCAGAAAAGGATCTGACA
AAACTCAACAGTCATTTGTGAGAAAAACTGTCAGTAAACTAGGAATAGAAAGTAGCTATCTCAAGTTGTT
AAGGACGTTTCAGAAAACCCTACATCTAGCCGGGCGTGGAGGCTCACGCCTGTAATCCCAGCACTTTGTG
AGGCTGAGGCAGTGGATCACTTGAGGTCAGGAGTTCGAGACCAGCCTGGCCAACATAGTGAAACCCCGTC
TGTACTAAAAATAGAAAAAATTAGCTGAGCGTGGTGGCAGGCGCCTGCAATACCAGCTAACAGGGAGGCT
GAGGCAGGAGAATTGCTTGAACCCGGGAGGCGGAGGTTGCAGTGAGCCGAGATTAAGCCACTGCACTCCA
CACTTCAGCCTGGGCAACAAGAGTGAAACTCTGTCTCAAAAAAAAAAAAAGAAAAAGAAAGAAAACCCTA
CACCTAATATTTTACTTATCAGTGAAATGTTGAGGTAGTGAATTCTTGCCCCTTAGAAAAATTGAGTGCT
TTCCCCTTAACATAGACAAATATGTCTATTTTTACCATTTTTATTTGACCTTGCACTGGAAGTTTTATCA
ACTGAACTAAAGGAAAAAAAAACAAAACAAATAAAATGCATAAATATTGAAATGTGGTAAATTATCTATT
TCATCAACATGATCATATTGTAGACAATCCTAATAAATCTTTAAAACTGATTAGAAATAAACGCGATATT
AAATATTTTATTTACCATAATATCAAAAAACACGAAGAATATAGTAATAAATTTAACAAATCATTTCGAG
ACACCTATTACTAAAAACCTCAAAACATGGCTGAGAATAATTACAGAAGATTTAAACAAATGGAAATATA
TATGCCATGTTCATGCAATGGAAGATTCAATATTATTAAGAAATTAATTCTACCCAAATGTATCTATCAA
TTCAACCCAATCCCAGTAAAAATGTCACAGATTTTCTTTGTAGAAATTGATGAACTGACTTTCTATGCAA
ATACACAGTGCTTAAAATTGCCAAAACAATACTGATAAAAGAATAATGAAAACATTAACCTACCTGACTT
CAAGGCTTGTTATAAAGCTGTACTAATCAAGAATCTACAGTATTGGCATAAACAAAGATATCAATGGAAC
ATAATGCTGAGTTCATAAATAGATCCAAACTACATGTCAATGGATTTTTTGAAATAAACACTACAGAAAG
GAAATAGAAGAAAGGAAGTGTTTTCAACAAAATATCAGGAACACATAAATAAATGTATGGAAAATAAATG
AACCTCCACTTCGATCTTATGCCAAGTGCCAGAATCAATAAGGATTGTAGACTAAGCAGAAAAGCTAAAT
CAATAAAACATCTGAAATAAAACACAGGAGAACATCTTTGAGACCTTGGGGTAGGCAAAAATTTATTGGA
AAACAGACAAAAAGTGCTATCTATTATTTAAATGTCCATGAAATTTCAAACAATGGAGACCTACTGAACA
AAAAAGAAAAGTCTAGTAATATACACATGGGTGAATTTCAATTTGAAGTATTCAAGTAATATTTGGAAAA
GTCACTAAGACTGTCAGGTCTCTGAGGTAACCACCTAGGTAGAGAGAGATTCCATTTACTGAGTTAGCAA
ATATCAGAAGAGAAGCAAAACTGTGGAGAAAAAGCAGTAAGATGAATTTTGGACCTGTTGAATTTGAGAT
GCCTGGGAGATAGTCTTTCAAATGTAGGTATTGAATAGGCAGGTGGGTATGTATTTCTAGAGACTAGGAG
GTATGCTTGAACAGAAAAATAGATTTTGAAATATGAACTATTATAAAAATGTAACTTATTATAAAAGGAA
AACTAAAGTAAGAGGGGTCTAGACAGAGAGAGTTCTGAATAAATCCAGTATCAAATGATTTGTTAGAGGA
AGAAAATCAGGTGAGTAGCATCCAGGGAGGTGTGGATCACAGAAGCTAAGGGCAGAAAATATTTCAATGA
ACAAGGAACAGAAAACAATGCCTGAAACTTCTAAAAGGGCAAGCAAGCAAGATAATTGTTTAAAAAATTT
CGTTTGGATTTATTGGTGATGTCGGTCTTGTTGGTGAAGTTTGTTAAAGCCATTTGGTGGGTTGTGGAGT
AAGGTGAAGAAATGGAACTGGCAAGTGTAGACAAGTATGCAATCTTCGAAAGAAATCTGGCCATAAGGAA
AGGATAGATGGTGGCACCTGGAAGGGGAAATAGAGTGCAAGGAGAGTTTCTCTTATGACAGTGGTGATAT
ATATATTTTTTGTTTGTTTGTTTGTTTTTTGAGACGGAGTCTTGCTCTTTCGCCCAGGCTGGAGTGCAGT
GGCGCTATCTCGGCTCACTGCAAGCTCCGACTCCCGGGTTCATGCCATTCTCCTGCCTCAGCCTCCCGAG
TAGCTAAGACTACAGGCGCCCGCCACCGCGCCCGGCTAATTTTTTGTATTTTTAGTAGAGAAGAGGTTTC
ACCGTGTTAGCCAGGATAGTCTCGATCTCCTGACCTCGTGATCTGCCCACCTCGGCCTCCCAAAGTGCTG
GGATTACAGGCGTGAGCCACCGCGCCCGGCCCACAGTGGTGATATTTTTAAGGGAGAGAAGGACTCGGTA
ACTGTTACTTTCTATAAAGAAACGGGAAGTAGCCAGTAGTAGAAATGTTGGTTAATTGAGAATGAATTCC
TTGCAAAGTCCAAAGGAAAACACATAACACAATTTGAGAGATTAGCTCAAAACAGGGGCACTTCTTTCAT
TTTAACAAGAAGAGAAAGGCAAAGCACAGTTACGGATGTAAGTAGTCTAGTAGATAAAGGGAAACAAAGT
TCAGGCTGGCTGACTTTCATCACCTCTAAGACGCTGAAGTACTGAGACTGTGCTTCTCAGATGGGAAGGC
ATAAGCAAAAATGGCTGAGGTTATGTGCAGAAGAGAAAGTTTGAAATAGTTTTAGATAATAGAAATGGAG
AAAGGAAAATACTGCTTCCCTTTCGCCAACAAAAGGAAATTTTTTTAAGAGTTCTTACTATCTGTAGAGC
TAGCCGTGAGCATGTTTATTACAGCTTACATGACATCTTAGCACCAGTCTCATCTGCAGGGCCAAGGGAG
GGGACCAATCCATTTGGCTTGGTGATGGAAGCCCGCACTGCTAGGTAATCATTTGGTAAGTTTTTGGAGG
GCTAGAAAGATCAGAGACAGAGCCAAACAGTTGATCACAATGAGTCAGTTGCACCTTTCATATGAAAATA
ATATTAATTTTATTGACTTAATCCGTGTACTCTTTATCATTTGATAAACATTATATATAGTGAACAATTA
TTGATTTGAATGCAAAGCATTTGTAGATACTAAGTTGTTGGACCTAAACCAATTTTTTAAAATCAGAATT
TAATTTATATTTGTTGGGAGTAAATTAAGTTGCTCAATAATTATTCGTGTTTCAAGAGTATTTGCTCATA
TAATGAACTACACTTCTCATTTAGGTCTTCACAGGGATCTTCACAGCAGAAATGTTTCTCAAGATAATTG
CCATGGATCCATATTATTACTTTCAAGAAGGCTGGAATATTTTTGATGGTTTTATTGTGAGCCTTAGTTT
AATGGAACTTGGTTTGGCAAATGTGGAAGGATTGTCAGTTCTCCGATCATTCCGGCTGGTAAATTAACTG
GGAGTGTTCATAAAATGTACTTTGTAATTAATTAGTCTTCATTCTCATCTAGTAAAAATGGCAAGATTTC
CCATCATTATAATATTATTTGAATACACTTCTAAAACAAATTGGATTGCCATACCACCAAATGGTAGTTT
CTTCTTCATCATAGCTTTAATAAAGTTCACTTAAATGAATAGTCTACACTTCTCTTCTTAGTTATTGAAT
GGAAGGCTAATAGAGAGGAGGAAACAGGGAGTCACAGATAAACTCGAATCACAATTAAACAACACCATAG
TCAACTCTCAGTTATCTGAGGTTTGCATAACTGCGTACAAAGCTTCCTTGGGACCTAGGATGAGCTCCCC
TTTCTGCCAGGAACTAAAGAATTATGGAATTGTTCATTGCTCACCTTGTCCCCGTAGAGGAAAGAGTTAA
GACAGGGGATAGTGTACAAAGGAGAAGGATAAGCAAACAGAGCTCCCCATATGACTGCTGCCACATCAGA
AAATCACCAAATCACTCTTTGAAAGAGTTAACTGTACTATATTTTGTTAATTTTAAAGAAAGTATCTTTC
TTTGATCTTTTATAAAAACTATTAGATCTTAAAATTCAGAGATAAAATATCACTTGACACATTTCCAGTG
AAAGTTTGATATGTTTTGTTATACTATTACTTTGAGTTGGCTCTAAGTTAGTGATTTATTTTCAAATAAC
AGAGGCTGTACACGGTTACTAAGGACACGTTCCTATAGATGATTTACCTTAGTAGTGATTAGGCTGAAGA
CTTTTTCATGAAATCTGTTTACAATTTCCCTTTCTGCTTTCAATGTTCAAATTTGAGTTGTAATCCTTAG
AACTATATTTCCTTCCCTAATCCTCAAAGATAGTTATGAATCTAATTTGAATCTAGAAGGATGCAAAAAA
CAGAACAAAAATTTAAAATGATAAAACAAGTAATATGGGCAAGAACTTAAAAAAATATATTTAGTAAACC
TTCATGATAGTGTGATGCAGTTAAGGGAAATAGGAAGCATAGTATCACTAGAATCTTACTTAGTGTGTCA
GGCTCTTTTGCATAAATTATTCTCTGGAATAAATTAAATACTTTGGTGCATGTATTTACTCCTTTGGGTC
ACTTTGATGCCATTAAATAATGCACTACTTTCAGCCTGACATTTACTGAAGCATCAGAAATAAAATGCTG
CTGCTCTTTAACCATAAATGGTACTTCAGTGAACTCTAAAGCTAATACAACCAATATGTCAAACACAATG
AGAAAGACATTTACACACTACACTGAATTAAGTCTATGAAGATATAAAGGTTAAAAAGAAGCCTAGCGTT
TTACTTAAGTTTAAGTATTTTTGTATTTGAATATAATATATGTTTAAAATATAGCCTAAAGTTACAGCAA
GCTAAAGATATAGCTAGATTAAAACAATCTAAAGACAAAGAAATTAGTTCATTTCTGCTTCCACTTTATG
TAATTTAAGTGTTGATATTATTCTCACCTGTGCATTTCAGCATATTTAAAGTACACTGAAAACTATATCT
GCTTTGGCCTTTTAAAAATAATGAGAGTTCCTACTTCTCTGAAACTGGATCTCTGCTAATTAACCACCAT
TAATCTGAAATATCTTAATTCCTTAAGGAGAAACAAAAGTGTATATTACATATGCTTATGTAGGATACTT
GAAAATTTGGTGTATCTTATTAAACTGCCAATTTAAAAACTGTATAATTTAATTATTTCATTTACAGTAT
GGACCATTTCAAAATGAAAAAAAGAATGCTCTATGGTAGCAAGTCACTGCTATATTTGTTAGTGATCATT
TGACAAATAAATAATTCATCATTCTATAATTGAGACAGTTACCTGTACATTTGCCCTGTTAATAAAATTA
CAGATTTTTCCCTTCCTGTGTCCATGTGACTAACCTGCACATTGTGCACATGTACCCTAAAACTTAAAGT
ATAATAATAATAAAATAAAATAAAAATAAAAAATAAAAAAATAAAAATAAAATAAAATTGCAGATTTTTT
TAGAAATGCAGAGCATTAACACTGTTCTTGCTTTTATTTCCAGCTCCGAGTTTTCAAGTTGGCAAAATCT
TGGCCAACTCTAAATATGCTAATTAAGATCATTGGCAATTCTGTGGGGGCTCTAGGAAACCTCACCTTGG
TATTGGCCATCATCGTCTTCATTTTTGCTGTGGTCGGCATGCAGCTCTTTGGTAAGAGCTACAAAGAATG
TGTCTGCAAGATTTCCAATGATTGTGAACTCCCACGCTGGCACATGCATGACTTTTTCCACTCCTTCCTG
ATCGTGTTCCGCGTGCTGTGTGGAGAGTGGATAGAGACCATGTGGGACTGTATGGAGGTCGCTGGCCAAA
CCATGTGCCTTACTGTCTTCATGATGGTCATGGTGATTGGAAATCTAGTGGTATGTAGCAAAAACATTTT
CCTCATTTTCATTAAAAGATAATGTAATCATTAAAAAGTGTGTTCAACTGAAGAATATTTTGTATTTTTT
AAATCAAGGCCACTTCCTATTGTCTATTACTCATGACTGTAAGAGCCATGTATAGTTTAGACCATTGTAA
TCCACACAAACCCTTAAACTACCTTTTGAACCAAAGTTATTCTTTCTTTCATTATCCTTCTTGCTACAAG
GAGAGAAACTTTTCTGTTATTTATCTTTCAGTTCTTGTACTAGAGCATGGAAGTGTTACTTAGAACACTC
ATTTTATTTATAAGTACTAGCAATAACACCTGAAAACGTTTCAGATTTGGTTTTCTACAAATTTAAAAAC
TAGCAACAATCTCAGTTTATTAAGAGCTCATGGGGTTTTCGGTGCCTAGAAACTATGGTATGAGCAAGTA
ACATTGTCTCTAAAAACATTAATTGTCATTTCTGCATAAAATTAACCACCCCTAACACCATATATATTTA
GGATAGTTAGCTCTTCTTGTTGCATTGATCCCTTTTACCATTATGTAGTGTCTTTCTTTGTCTTTTTTTA
ATCTTTGTTGGTTTAAAGTCTGTTTTATCAAAGACTAGGATTGCAAACCCTGCTTTTTTTTTTTCTTTCC
ATTTGCTTGGTAAATATTCCTCCATCCGTTTTTTTTGTGCCTATGTGTGTCTTTGCATGTGAGATGGGTC
ACAGCACACCGATGGGTCTTGACTCTATCCAATTTGCCAGTCTGTGTCTTTTAATTGGGGCATTTAGCCC
ATTTACATTTAAGGTTAATATTGTTATGTGTGAATTTGACCCTGTCATTATGATGCTAGCTGGCTATTTT
GCTCATTAGCTGCTGCGGTTTTTTCATAATGTTGATGGTCTTAACAATTTGGTATGTTTTTGCAGTGGCT
GGTACTGGTTTTTCCTTGCCATATTTAGTGCTTCCTTCAGGAGCTCTTGTAAGTCAGGCCTGGTGGTGGC
AAAATCTCTTGGCATTTGCTTGTCTGTAAATGATTTTATTTCTCCTTTGCTTATGAAGCTTAGTTTGGCT
GGATATGAAATTCTGGGTTGAAAATTCTTTTCTTTAATAATGTTTAATATTGGCTCCCACTCTCTTCTGG
CTTGTAGGGTTTCTGCCGAGAGATCTGCTGTTAGTCTGGTGGGCTTCCCTTTGTGGGTAACCCGACCTTT
CTCTCTGGCTGCCATTAACATTTTTTCCTTCATTTCTACCTTGGTGTATCTGACAATTATGTGTCTTGGG
GTTGCTTTTCTCAAGGAGCTTCTTTGTGGTGTTCTCTGTATTTCCTGAATTTGAATGTTGGCCTGTCTTG
CTAGGTTGGGGAAGTTCTCCTGGTTATCCTGAAGAGTGTTTTCCAACTTGGTTCCATTCTCCCAGTCACT
TTCAGGTACACCAATCAAACTTAGGGTTGGTCTTTTCACATAGTCCCATGTTTCTTGGAGACTTTGTTCG
TTCCTTTTCATTCTTTTTTCTCTAATCTTATCTTCATGCTTTACAAATTTAACTCAACATGGATTAAAGA
CTTAAATGTAAGACCTAAAACCATAAAAACCTTAGAAGAAAACCTAGGCAATACCATTCAGGACATTGGC
ATGGGCAAAGACTTCATGACTGAAACACCAAAAGCAATGGTAACAAAAGCCAAAATTGACAAATGGGATC
TAATTAAACTAAAGAGCTTCTGCACAGCAAAAGAAACTATCATCAGAGTGAACAGGCAACCTACAGAATG
GGAGAAAATTTTTGCAATCTATCCATCTAATATCCAGAATCTACAAAAAACTTAAACAAATTTACAAGAA
AAACACAACCCTATCAAAAAGTGGGTGAAGGATATGAACAGACACTTCTCAAAAGAAGACATTTATGTGG
CCAACAAACATATGAATAAAAGCTCATCATCACTGGTCATTAGAGAAATGCAAATCAAAACCACAATGAG
ATACCACTTCACGCCAGTTAGAATGGCGATCATTAAAAAGTCAGGAAACCACAGATGCTGGAGAGGATGT
GGAGAAATAGGAATGCTTTTACATTGTTGGTGGGAGTGTAAACTAGTTCAACCATTGTGGAAAACAGTGT
GGCAATTCCTCAAGGATCTAGAACCAGAAATACCATTTGACCCAGGAATCCCATAACTGGGTATATACCC
AAAGGGTTATAAATCATTCTGCTATAAAGATGCATGCACACGTATGCTTATTGCAGCACTATTCATAATA
GCAAAGATTTGGAACCAACCCAAATGCCCATCAATGATAGACTGGATAAAGAAAATGTGGCACATATACA
CCATGAAATACTATGCAGCCATAAAAAAGAGTGAGTTCATGTCCTTTGCAGGGACATGGATGAAGCTGGA
AACCATTATTCTCGGCAAACTAACACAGGAACAGAAAACCAAACACTATATGTTCTCACTCATAAGTGGG
AGTTGAACAATGAGAACACATGGACACAGGGAGGGGAACATCACACACTGAGGCCTGTCGAGGGGTGGGG
GGCTAGGGGAGGGAGAGCATTAGGAGAAATAACTAATGTAGATTACGGGTTAATGGATGCAGCAAACCAC
CATGGCAAGTGTATATGTATGTAACAAATCTGCATGTTCTACACATGTATCCCAAAACTTAGAGTATAAT
AATAATTTAAAAAAATTAACCATACCCAACACTAGTGTCCTGAATCTTGAAGGCATGGAGAAGTTGGGAA
GGCATGGGAAGATAAATATAACAAAGTGATATAACATGTACTCAAATAGAATTAAAAATAGGAAGTAACT
AATATGTGTCCAAAAATATGAAAACAAAGTGCCATGTGTCAAGTTTACAAAATGTAAACCTTGCTTTACA
ATAGGAAGGTTGATCAGGGAAGTCTTTGTCAAAGAGTTTGGACCTAAAATATATTTAACTGAGATGTAAG
ATTTAGCTTGGTAGGAAGAAAGACCATCCCAAACAAGGAAACAAGGTACCCAGTGACTGAGGGATACAGG
ACAGTAGACTCTGTGAGAAGTATCAGGCTCTTATGCTTTAAATATGAAGTAATTACACCGAGTTGCTTAA
TTAGAACCCAAACCAATGGAATAGAAAAATGACTACCATAACAAGTAATTTAATGTATATACTCTTGCCA
GGCTCAGTGGCTCACGCCTGTAATCGCAGCATTTTGGGAGACTGAAGTGGGCGTTTCACTTGAGGACAGT
AGTTCGCGACCAGCCTAGTCAACATGGCAAAACCCCATCTCTACTAGAAATACAAAAATTAGCCAGGCGT
GATGATGCACACCTGTAATCCCAGCTACTTGGGAAGCTGAGGCACGAGAATTGCTTGAGCCTGGGAGGCA
GAGGTGGCAGTGACCCGAGATTGTGCCATTGCATTCCAGCCTGGGTGAAAGAGCGAGACTCTGTTGCAAA
AAAAAAAAAAAAAAAGCATATACTCTTTAGACATGATTTCCTCTCATATAAAGGTAACCTCCAAGTCCCC
AAAGATAGAGAAAGGGGAAGGGAAAAAGGCAAAGTATTATTTTATTTTTATTCATTGCCAAATTTCAGCC
TCTTCAACATTACTTTTGATAATTCTGATCTATTTTTAAAGTAACAAGAAACATAAACAGTGTACAATCT
AGAATTATAAACAGTGGCTTAAAACAATAAACACTGATTACTTCATAGTTTCTGTGGGTCAGGATTTGGG
GAATAAGTTAGCTGGGTGGTTCTGGTTTAGGATCAGTCATGAAGTTGCTGTCGAGATGTTAGCTGAGGTT
ACAGTTATCTTGACTGGGGCTGGAGGATCAGCTTCTAAGAAGGCTCAATCTCATGATTATTGGAAGGAGG
TTTCAGTTCCTTTTTGGCAGTTAGTTGAAGGTCTCAGTTTTTCTCTGCAGGACCTTTTCCATAGGACTGT
TGAGTGTCCTTATGATATGGCAGCTGGCTTCTTCCAGGGAAGGTGATGTAAGAGAGAAGGCAAGGAGAAA
ATCCTCTTTATGTTCTACTCTTGAAAGTCACTCTTCACCACTTCTGCCATATTGTATTCATTAGAAGCTA
GTCACTAAGAAGAGCTCAAGCTACTATAATCCCCAAGACAACTTTAAAATGTTTGCTTTCAGAAAAGTAT
AAGATCACATAGAACAGAAAGTGCCATAGGGTTACATAGAACAGAAACAAAGAAAAGATAATATAATTAT
GTTATAGATTTGATTTCATTTTCTCTGTATGTATATTTGGTATATGTTGGAAGAAGAAAAGAAAACGCAG
AGAACAGAATCCTTTATGACAACATGAATGATCAGACAGCAATGGGGAATTAAGAAATATAAGTTTGGGA
CCAGATTGGGTAGAATTTAATTTATGAAAAGGCTGACTGTGCATAATAAAATGTATTTTCCTTTAGGCAA
TTCAAAGCTACAGATGATTTTTTATCAGGAAAGTGACAGTGAACCAGTGATATTTTTCAGAAATATACGT
AGCAGGAGAATGCAGAATAGATTTAAAGAGGATGAAACTCAGCCCACCACATGTTATCTATTAGTTTACT
GAAATTAACATATCTCTCTAATGTATAAATGTGCAGAAAATTGAAGTTGAAAAGAGAATTTCAGGAAATA
TCAAGTACTTATGGTTGACATCAGTATTAATTTAGATTGTGATGTATGCATAAAAAGATATAGTTTATAA
AATAATCATTTCCATCTACTGGGTGTAAATTTAATTTTTGTTCTTTTAAGAGAGAAAAATTAAAGGTTCT
CCTTTCTTTTTGACTATCAGTTAAAATAACTTCTTTGTCTTGTGATAACCTGGGTATGTTTCTGGAGTAG
CTAAGGTAGTCATATATATCATGTTTACCACTATTAAGGAAATGTGCTTATATAACATTTGCTTAAGACT
GAATGAACTTGATATACTCACTCCTTACTACAATTCTTCCTTCCTATTCTCACTGGAAAAATGGGAAAGG
TGTCCCAAAGACAAAATGGCATAACTTCCTTTTAACACACATGAACTATCAGATGTGGCTCCACCCAAAT
AGATGTAGTAGTCACAATGGATGGGACTGCCAGCCTAGTCTACAGACAAGACAGAGCTGGGACCACAAAC
TACTGTTTCCCAGACCAGGATTTTTATGAGCCATTCTTAGTTTCCAGACACGATGGCAAGAGACCCTTCA
TTGGTTGAAGATAGGTGCTGCAGAAAAAGAATGTGACTTTCTGAAAACTGATAGTTCTAGAAGCAGAGAA
GACAACTTCCTCTCTCCCTAAGTGAAGGTGAGGCAATAGCACACAGGAGGGATGTGAAGGTTTTGGCTTC
CTCTCACAAGTTGGGAATCAGGATGGAGAAACAATTAAAATATGTAATATGTTTCAACCTTGAATTCAAA
ATGGAAATTATGGTAACATTTCCATTCCAAGAGGCTAATTTGAGACACAAGAAAGAGTTGATTTCATTTA
CTGAGCTAGCACATTTGTGAAACAGGATTCAGGATTTCAGTCCCTGAGTGAGCTTGCTGAACTGTTTTCT
TTCTTTTTTTTTTTTTTTTTTTTTTTTTTGAGACGGAGTCTCGCTCTGTCGCCCAGGCTGGAGTGCAGTG
GCGGGATCTCGGCTCACTGCAAGCTCCGCCTCCCGGGTTCACGCCATTCTCCTGCCTCAGCCTCCCAAGT
AGCTGAGACTACAGGCGCCCGCCACTACACCCGGCCAATTTTTTGTATTTTTAGTAGAGACGGGGTTTCA
CCGTTTTAGCCGGGATGGTCTCGATCTCCTGACCTCGTGATCCGCCCGAGGCCTCCCAAAGTGCTGGGAT
TACAGGCGTGAGCCACCGCGCCCGGCCTGAACTGTTTTCTTAAATTGTCATGGATCACACCAAACACCTG
TGCCAGCTGTTATGCGCATACCCTTCGGTAACAAAGGAAGTCCAGAAAAAGAGAATAACTTGACTCACAC
AAATATTTCTAGGGAAATAAGGTAAATAAAAAGATAGTGTTGTGGAGGATAAGTTGGATAATAGTAAGTG
ATAACAGCTAAACTTTCTCAAAGGTTCACTATGTGCCAAGAACTGTGCTGAAAGCCACATGAATTCTGTC
ACTGAATCCTTTCAACAACCTTGTAAGCTGGGCACTAGAAAAACGATTACATTTATTTTATAGATGAGGA
AACTGAGGCTCAGATTGGTTATGCTACTTAGTAGGTAACAGAAAATCGATTCTTACCTAGCACTCGAATT
CTAAAATATGTGCTCCTCTATGTCAAGTAATCTATAGAACTAAGATAAACATGCTGATGAAAGTTAGTGT
CTAGTGGGTATTAATAAACGCGGTTTCAAAACTGTGTCACCACGGGTAGATTGGCTGCTTTAAAAAAAAT
AAAAACTTCAATGGATTTATGAGAAAGAAAAGTCATATGTTCCAGGGATATTTATTTATTTTCCTGACAG
TGGAATAGCATTGAATTGAGTTATCAATTCATAAAAGATCAGAGAAACAATTCGAAAATTAATAGTAAAC
CCTATTATACTGACTAAATATGGTAGCAGTTCAAAGAAAGGAAGTATCGGTAAGAGTAAACATGGAATAC
TGTTTCCTCACTTATTCTGCAAACATCACAATTAGGAGAAAAGACCTTGTAGTTAGACTTTCAAAAAAAA
AAAAGCTTCTCTACTTATTAACTGTGGTCTTGGACAAGTCATGTAAGTTGTGCAGAAGCATTTTCATCTG
TAAAATAGTAATAATTCCTGCCTTATAGAGTTGTGAGAAATAAATCACATAAACCTTGAAAATGCTTTGC
ACAATAACTGGTATTTACTAAGAGCTCAACTAAAAAGCTGGTTTTACTTTTATTGTTATTATGATCTGGT
ATTGATACTGCTCTAGGACTTGGCTCTAAGGCATGTTTCTGACCAAAAGATCTCCCAATCTATCAGTAAA
GCTGTGCTTGTTTTTTTTTTTTTTTTAAAGAATCCAATATAATGTGATAGGGATGTGGATGAGAAATTTA
ACAGGACAGAATGAGAAATGGGAGCAGGCTATATAAATGTCACAGTAAATGACATTTGTAAATAGAGTAT
TTGTCCAGTACAGAGGCAATATAATTGGCTCCCGCCCTGGGGAAGGATTGATGGATGTGTATCAAGAAAA
ATTTCCAAATAGACAAATGACAGAACTTTAATATACTTTAGGAAAATAAGTCTAGGAAATAGCACCAAAA
TAGATAAAATAAAAATTTTACATGCAATTTTTTCTTCCTTTGTCTGTTTTTTTAATCCAAATAATAAGTT
CAAAAGCAAATTACAATAAAACATAATTTTATTGGTAAATTCCAGAGGCAAAGGAGCAGGTCTGGTCTTA
ATGTGATTATCAGGAGTCATAGTATAGAGACTGACAGATTGTCAGTACACTCTCAAAATCAAACGTGGTC
TTCATTGGATCTTACATATTTTTACTTTAAAAAAAATCACCATTGGTTAGACTAACTTACAACTAATTAG
ACAAAGGTGCTGTAAGCCTCATTAGCATGATAGAAGCATGAGAATATAGCAAGAATGTAGAATCCTTTTT
ATTGAAGTTTTACTTAAAAATTTTCCTAAGATTCTACTTTTGTACTACAGTTTGAGCATCCCTAATCTGA
AAATTTGAAATCCAAAATGCTCGGAAGTTCAAAGCTTTTGAGCACCAACCTGATACTACAAGAGGAAAAT
TCACATCTGGCCTCCTGTAATGAATCGCAGTTAAACCACAGTCAAAATGTTGTTTCCTGAACTAAATTAT
TAAAAATATTGTATAATATTACCTTCAGGCTATGTGTATGTGTATGAAACTTAAATGAATTTTATGTTTA
CACATGTGTCTCATTCTCAAGACTTGTCATTATGTACATGCAAATATTTTAAAATCTAAAATCCAAAACA
CTTCTGGCCCCAAGCATTTCAAATAAGGGATATGCAACTTGTATTTACTTTGTGCATTTGCCCCCCTTTA
CTGCTATATCTTTCTTTTGTTCTGTATGTTATGTGTGCTTAAATAATCAGGAATTCATTGATATTGTCAA
TCAAATCCTGAAAAAAAATTATATGACTCAGTCTTGTACCCCTGAGAATGTCTGATTTCTTCGTAAGTTG
TCTTTTTTTTTTTTTCCACAATAGTGAGTTTAATGTCATGAATCTTTTCACTCATTCATACTGGTGGAGC
CTATTTTTAAAGACCCAATTTGCAGACTGATTACTGTCCTTATTCATGGCAATACTTCAACTCCACAATC
TTTAATTCAACAATAACATCATAATTATTGTATAATAACCATTTTATAGTATTTCTCACTATTGTATAAT
TATAGTAGCCATAATTGTCTTAATAAAAATTGGGACTTTTCATCCAGCAATAAATACGTTTTTGTCTGAT
TTGTCCAGTTATCTAGGTACAAAAAATGGTACAAAGGCACAAAAATAAAATCATATTTAAATATATTGGG
ATAATTGTTGATTTTAGGAATAAATTATCAGTGTTTCCGGAAATCCAAATTACATAGTCAAAATAGCATC
TGTATTAGGCCATTCTTGCATTGTTACAGATAAATACCTGAGACTGGGTAATTTATAAAGAAAAGAGATT
TAATTAGCTCATGGTTCTGCAGTGAGCTTGGTGCTGGCATCTGCTTGGCTTCTGGTGAAGTCTCAAAGAG
CTTTCAATCATGGCAGAAGGCAAAGTGGAGCAAGCATTTCACATGATGAAAGCAGGAACAAGCAAGAGAG
AATGTGGGCAGGAGGCACTACAAACTTTTAAATAACAACATCTCATGAGAACTCACTAACATGAGGACAG
CACCAAGGCATGAGGGATCTGACCCCATCATCCAAACACCTACCACCAGGCCCCACGTCTAGTGCTGGGG
ATTACAATTCCACATGAGACTTGGGAGGGGGCAAATGTCCAAAATATATCAGCATCCCAAATAAAAGGGT
TTTTTTTGTACAGTTGTCTATATTTATCTTTTGGAACTGAGCTTAATAGAAATGTTTCATTTAGCAATGA
TTTCAGTATTTTCTGCAATGACTAAAAAGCAAATAGTGATAATAGTATTATTTTATATTGACCAAGCATT
TTTATTTCATTCACTTTTTTTCAGAATAGTGTATCATGAATTAGCAGAAATGCATGTTAGAATAAAATAA
GGTGTCAAGAACAATCTTAGAAAACTAATGATGGAAAGCAATTGAAGCAATAGAATGTTTTGATCACCTG
TTTTTCCTGCTGTGTTTCAGGTTCTGAACCTCTTCTTGGCCTTGCTTTTGAGTTCCTTCAGTTCTGACAA
TCTTGCTGCCACTGATGATGATAACGAAATGAATAATCTCCAGATTGCTGTGGGAAGGATGCAGAAAGGA
ATCGATTTTGTTAAAAGAAAAATACGTGAATTTATTCAGAAAGCCTTTGTTAGGAAGCAGAAAGCTTTAG
ATGAAATTAAACCGCTTGAAGATCTAAATAATAAAAAAGACAGCTGTATTTCCAACCATACCACCATAGA
AATAGGCAAAGACCTCAATTATCTCAAAGACGGAAATGGAACTACTAGTGGCATAGGCAGCAGTGTAGAA
AAATATGTCGTGGATGAAAGTGATTACATGTCATTTATAAACAACCCTAGCCTCACTGTGACAGTACCAA
TTGCTGTTGGAGAATCTGACTTTGAAAATTTAAATACTGAAGAATTCAGCAGCGAGTCAGATATGGAGGA
AAGCAAAGAGGTAAAAATGTTTAAATAAGGAGATATTTTGGTGTTATATAATTCTGTTGTTTAAAATTAT
CAGGTGTTTTTAAATTGCGTGTTTCCTTCCTGTTAAGAAAATAGAAAATATCTGTCTAGCAATATATTTT
CCATGGAAAAGTTGGTAATAAATAAATTAATGATAGATTAAAATATAGCTAGATTAACAATATGCTGACT
TATGTTTCCAATACTGACATTTTGAATTCTTGACAGTATTCTTGATATGAATTTTTTCAGTATTTATAAA
TAATTTTAAATTTCTCAAAATGCCTCAATTTCTCCACTTTCTTCCTTGTAATTTGCCCACAACAGTGTTT
TTTGTACGTACTGGAAAAATATCTGATGAGAGGGTAGTTGCAATTCTCATCTTGCTATGTTCTTAGTTCT
TAATTCTTACGAAATACGTCATAAAATAGTATTGTATTTTGTTTGCACAGACATATTTACTCAAGGAAGA
TCTGATTGGGATCTTGGCTTGATATTTATGTATAGTTTATCTTTCCTGAAGTCAGTCAGTTTTTTTGAAG
AGAAGGTATTGATGAGGAATCACACTAAAAACATATTTAACCCTACTGAGCTCAGTGTTCACTGTTTAAA
GAAACAAAAATCCTTAATACATTATAGAATGTAAAATTCTGAATTTACCAACTCAGTAAGTCCTGGTAAC
TTAATGTATTCTTTGATTTACAAGAAGGGTATGAGCAACAGAATATATTTTTTGTTTTGTTTGCTATTAA
CCTGTTGCTCAATAAGTACAGAGTTGGAGGTAAAGAGAGGAATTTAAAACCTTGATATTTAATTGTTTAT
ACAAAAATGAAGACAAGATTTCCAGTAATTAAAGTTTGCACTAACTAACAAAAATAACAAGGAAAAACAA
AGATTCGTTCCTTCCTCATACGAACTGTTTGGCGAGGAAGATAAAAGCTTCTATTCCTGATGTCGGGAAA
GAAAGAATGACGACATGGGGGAGTGTGGGCACTGAAAGGTAAAATTTAAGTAGCACAACATGATCATGAT
AATTAACAATCAGCCAAAATTATGAGGGAAAATATAGTTATAAAAAAAGAACAAAGATGGGTGGATCACG
AGGTCAGGAGTTCGAGACCAGCGTGGCCAACATGGTGAAACCCTGTCTCTACTAGAGATTCAAAAAAATT
AGCCAGGCGTGGTGGTGCGTGCCTGTAATCCCAGCTACTCGGGAGGCTGAGGCAAGAGAATCGCTTGAAC
CCAGGAGGCAGAGATTGCAGTGAGCCGAGATCACCCCATTGCACTCCAGCCTGGGCAACAGGATGAAACT
CTGTCTCAAAAAAAAAAAAAAAGAACTAGCTATTTCAGACACTTTTTCTGTATTTATTTGATAAAATTAC
TAAAGAGTATGTTATTTTCCATTTTTTCTTGTTTGTAAGTTACGTAGTATTGCTGTTAGTGATTAGGTAG
AAGTAGATGTTTAATGGGAAATTCAGACAATCTTTGAATATAGGAAGGTATAAATAACAGGGACATAGGT
ATCAGTTTCACAAGAAATAACTGATGAGATTCAAGGGAAAAGTAATAAAACCTTCTGTCCTGGGGCAAAG
AATTACTTTAATTGGTTGAACTTAAATTTTTACTAACTAGATTATTGTTTGAAAGTTGAATAATATCTTA
AAATCTTATTAACAAAATTTTGAACAAGTGTTGTTACAATAGTTGGGTTATGCTGGAAGGGTGGAGTGGC
CCAATTTCATATACAGTGTACTGCTCTTATAGAAGCTGAAGTCGGCATTTATAAAATAGAATTCGGTCAT
TTGAATTTTGATGTATATTCCCCTCTCATTATTTTGAAATTATGCCTAATGGTGAATATTTCCCTAATAG
TAAAAAAAGTCAATTTTTATTTTCACACATGTTTAGTTTTAGGCTGTCATATAAACTAAGAATGAATTAT
ACAGTATCAAACGTTGAAGCCATTGGCTAGTTTAATCTTTTAGCTAAGTTTCAGTATCTTTTGAGGAATG
TTTAACTTGACATCCAGTCTTCTTAACTTTAAGAGATTTTACAGCCGTGGGTTTTCCAAAAGAGCGTGTA
TTTTGCCTTAACTTAAGCCATTATGTCTGAAGTAAGAGGGAAGTCCAGTGATGTGGGGTTTAGAGTAGGG
ACATCTCTTGTTTCTCTTGTTATCATTAAGCTTTTTGATTTGTTTTCCCATTAAGTTAGCTCTGAGTTAA
ATACTCTAAAATAATATTTGTGAATTCAGTATTTCAGAATTGGAGGAAGAGAACTGACCTGCCAGGTGGA
AGCAGACAGGATTATTTTATTGCTTGAGTTGTGGAGTCCTTCCAATACCTTCCCAGCATAGAGACTGTTA
CTTCAGTGTTAACATTATTTGGAGGGGTTTTTAATTCTGGCTTTATATCAAACTTTCTAGACATAAATTT
ATAAAATAATAAATGATGAGGGTTATCGCCGTGAAAGAGGTTATGTGTAGGTTTTGATCTTTCAGAATTT
TACCTGGTAGCTCTACACTAAAAAACTAGAGAATTAAAACAATTATTGAAGAATTTCAGACACTCGCATT
TGAAATAGCATTTCTTGCCTGCCTTCTAGTCATTTTTGTCTGGTCATTTTTCTAACTGGGGGACAGGATT
ACATTGTTAAATATCACAAAGTAGTAAGAAACATCATGAGGCTTATTACCAATCCTTTCTAAATTAATTT
TTTAATTAAAGAAAAAATGAGGCTTTTTTACTGGAATGTCTAAATGAATTTTTTTATAAGGCAGACTGAG
TGGACTCAGAGGTTTTTTAGGTGTTCACAGTAAGTCCTCTGCAATGTCTTTGCTAAATTTGTATGATTCT
TCAGTAGTTTTCTGTAGATTCTCTAGAGTAGGCCATTTAAAATCATGTCATAATCCCCTATGCTTTAATT
TTAATGTTATTTCGATTATATTAATGTAATTCCTTTTGTGATTTTGAATGATTGTTTTTTCTTTTAGAGT
ATTTAATAATGTGGAAGCCATGCTTGAATGACTATTTTTCGAAGTGAAATTTAGTAGTGCGATATGGTGA
CCTTCACCGCTTACCATTCTTACTTCTCACAGGAGTAAAATCAAGCTGGAGCCATCAAGAATGCAGCTCT
GGTGTTTTTTAACCAGCCAGAGGCTCGTGCCACCACTTTTACCCAGGTTACCCAAGCAAGTTGTACATCT
ATAAATATAATCAGTTTCTAAATGACTTTTGACTGGCCTGCATGTTACTCAGCTACGTTCCTTGCCCTTC
CATTGGCAGTAAAATAAAAACATGCACAGCTGCTATTATGCTGAGTCATACAAAGCATGGTCAGGCAAGT
CTGACAACCCTAACTTAAAAAAAAGTGATTTAGCTGCTAATTTTCTTACATAGATTTTAATAGAAATTTT
ATTCAATGAAAAGTAAAAGTGCATGCCTTTATGGATTATTTAATTTCCTTTTAATGTTACAGAGTTTTGA
ACATATTAGGAGCCCAAAGGAGAAATGTAGGTGCTCTTTGAAAACTTGCAAAAATGCTTTTTATCCTCTG
TCTTTAAAAAAAAGATAGCCCAGTTACTGTACTTAAGTCTTGACAGTTTTTTATTTAGTGTAATGTTTTT
CTGAAGGGTAATCTTCAAATTAAAGCAATCCCTTATTCATATGCAAACTTCCCAAAGGATGTTTTAATGT
GATAATAATGTAAATGAATAGGAATGTCTGTGTTTCAGTTGCTAGCAGCATGGGTATAATATTTATCTGC
TTCATTTTAGGGAAAATGGCACTGCTTTATTTAGGAGTTGACCAACAGTATTTTGTATTTAGAATATAAT
TTCTTTGGAAAGTCTGTTTATATTTACCCTTAAAACTCTTAGACTGAAAGAAAAGGAAATCATGTCTTTT
GTATACCAAATAATAATAATAATAGTGATAATGAGAGACTTATAGATGGTATGCTCCTTCTAAAAATAGA
TTTAGAGTCCATCTTCTTCATTTTCTTGGCTCCTCTGTGCTTTCTCTCCCCTCTATTTTATTGAGACCTG
CTGGAAAACTTTCTCCCGAAGAATATTATTTAAATTTATCATGATCCACAAACTCCTGTATAGGAAAAGA
ATCAGAAACTCTTGCTCCTAGGGTGTTTTTAAAATGAAGAGACTTCCCTATCATGTGACAATAGCAATAA
ACGTAACATCATTCTATGGGATCCATTAGTCGACCTTCATTTCTTAATGTTGAAATCACAGTTTTATGCA
CAAATATTTAACCAAAATGCCTAAACCCAATTTAATCATTTTTAAGAAATGTTAATTATTTTGTCACTTA
GATACAGTTTCCTCTCCTTTTGCCAATAAAACTATAAAACAGCACTAATATAAAAGTGTAGTTGGCTATT
TGGAAGAAGCAATAATCATGCCATTCCTGGAGCATTCTTTTATACTTTGGAACAAAATATTCCATCACTG
GCTCTCCAGATTCATGAGCTATAATGCCTCATATATTGGAGGAATGGGATGTAAAATGGGATCCAAGATG
CGTAATTGTTTACAGTTAAACACAGATGCGCATATACACAGGGACTACAGATAATTACTTTTTCCTATTA
TGTATTAATTCTTCAGAAAAGCATGAGATTTAGGCACTTTCGGATAATAGCTTGTTTCTCGGAAAGAGGC
AAGGGTAGTTTCCTTATTCTCTGAGTATCCCATTTTGCCAATTTCCTGTTTAGAAAGATACTTGAGGCAT
ATTATCCATCAACGTATCTAGGGGATTCAGCTGGAGTAAAGGTGGTAGAATAGAAGCTAAGAAGGAACTG
GTTCGTTTATTTTCAATCCTCACATTATGGCAATTTTTGATTTCCTTGTAAAAGTCTATGATTCTCCCTC
AGGAAACATTGTCCACTTCCTAAAAAAATATACTAATTTCTAATACAGGGGTTTGGAAAGGGGACAAAAA
TGTGCAGGGAAGGTTTGCGTAAGCAATGGTGGAATGGGTTCAACAGACACCTGTCTATGACTTTATCCTG
GAGAATGTGTAGTCCTCATGGGAAAGTTTTCCAGTGGGATAGTGATTAAGATGGAAAAAAATGCCCAAAA
TATCTTTAATATAAGAACAAAATGGGCCAAACACGTGTCTTTGGGTCACTGGTAATCTACTGAGCAGTAG
GACATCATGACATAAGAGTTCCTTTTGCCATCCGAAGAAAAATATTTAAAATCCTATTATTTGTGGTTTT
AAAAATGTTATAATGTATTCATTATAAACACTAAAATGACTTTCTGGATAATATAGTATACTGTGAGTAA
TTATTTTGATTTTACCATATTCTTTTTTAGTTCTCAGAAACCAAAATTGTCAGATATGGGATACTTGATT
TAATCTGTATTTGAAGTTTTCTCTTTTTTAAGTGCCAATTTTTTAATTAAATTAAATTAAAATCTCTCTC
TCTTTTCCCAAATTATATACAATATCTACTAATTATGTTTTCTTCGAAATGTATCTTAGCTTCATAATGA
GAAGTGAGTGTGCCCATGAAAAATTTAATAGGAAGTTATGTTTTCTCTTCCATTTTCTGTTGTGATTCAT
TATTTTTGAAAATAATTTACTTTCATTTGCTCACATTTGCTGTCTAAAGAAAAACTATTCATCTGGCACA
TTCATATTTAGTAGTATTATTAAAGCAGAAAGCATAAGTTGGAAGTATAATATCTAAAAATACAAAATGA
AGTATTGTACCTTGATGTTTATTAGATCATTAAGCAAAATATGATTCTGCCCTGCTTAAATCATTTGATT
ATAATTATCCAGCATATAAAAGAATCACAGTAGATTTTCAATAGGAAGAGTCCTATAATATTAGGTATCC
ACCAAAAACATTGTTCAAGTAATATTTCCACCTGAAAGTAAATGATTGCCAATGCTTTTTTTCAGAGCAT
ATAAAATTGGCTATTCCTATTTGATCTCGTTATTGTCCTGGCATCCTTATTTTGTTAAATTTTAACTAGG
CAAGGGCTATGCTACAAACATCAGTTAGTCCGCTAGTTTCCTGATAAATAAGTACAGGTAATTAAAAAGT
GAACCTAAATATCCAAATTATACCAAAGGGACATATATAGAACTTTTTAAACTGGATCTGCTTCTAGCCA
GTTCATATTTTGGTCACTTACTAATGTAGTATTTCACCATAAATTATGCCTAGATTGGAGCATTTACAGG
CACTCTTTATCTGAAAATTCTTAAGTGCATGAGTTGTAACAGTTTCACATAGTGATCATCTCACTGTTCT
AATTGGTGACTATGTACTACAGTTAGGTTGATCGTAATTATGACCTTAAATGAAGCTGAATATTTTTATA
TTCCTAATTTGATTAATTTTATTTTATGGGCTTTTACACATTTTAACTGCTTTAGTCAACATATTTTATA
ATATTATGACATTTGCCTAGAATGTAATTTTAAGAAAGTCATTTAATTGATGTTATCAAGAAGGGTTTTA
TAAATCAGAATTTCCTGCCTATGTTTCAAGATCATTGCTTAAAGAAAACTTTTTGTTCATGTAATATTCC
AATGTGTATTTAGCTTTACTCTACATTAAAATATGTTAACAACTATGAATATAACTTGAATTAATACTAA
AGTTCATGGTTTTGAAACATGGAAATCAACAATATCATAAGCACTATCTTGAACCTACAATATTTGATTA
CATATCTAGTCTACTAAATGTTTTAAATTGATAAACATTGGGTTTACTTTTGAATCATCAAAAAGATTCT
TTAGAGAAGCTTGGCAGAATGGGGTGCAGAAGATCTGGAATCCAACCACTGATTCGCTATAACTTTTCAC
AAGACGATAAAACTCACATTTCCTTCTCTAAATAAATACTGGATTGGCTGACATTAAGGATCAATGTGCT
GCCTAGATTCTTTGTTATTTGTAAATCAAGTACCACAAGTGGAAAAGTATTCAAGTAACACATGTGACAG
ATCCTGTGCTGCTCCGCTTCAGAAGACAGTGGGGAAGGATAAGATTGCATTCCTTAAAGAGGCCCCATTC
ATCACTGGAGCTATAGATCCTTGTATACAGAGTGAAAAGAGGGAAAACACTGTCAAAATGATTTAGTAAT
AGTTTTCCTGACTCCACAGTTAAACTACAGTTCACCTCATACACTCACATTAGGTCCGAATAATTGGCAG
ACTGGTTTTAAGACAATACTCCTAGTTCTAAGAGTTGTTCGTCATTGCCCACACAATTCAGAATCTTAAA
AGATTTGTGTTACTCTGCAATTAAGAGAAAATATTGTGTGTATTCTTTTGAATGTGAAAGTAAATATCAG
ATAGGAAGTGTTAGTAGTTAGTGGTTGGATAAAAGAGTCCTCGCACTGGTCATTCATTCATTTACCCAAA
ATTTATTAAACACAAACATTGGTGCTAGAGATACAAGGGTGACGAAAACATGATCTTTCCTCCAAAAAAT
TAAGTCTGATGAGATGCATTTTCTAGAAACACAAAATACTTTTGAACTGAACCTTGGAAAAAGTAAAAAC
TTGACCTTTCAATAGATAAATATTTGGCTTTAGGAAAAAGGTATCTTAATTCTACATCAGAACTAAGGTA
GTGCACTAAAATGAAAGGGAGCAATGTTAATTCTTCTACTTTTAATGTGATTTAAATAAGAGAAAATACA
GGAATGTCTTTTATAATTTGAAATTCCAGAGAAAATGAATAAAAAAGCAATTAAAAAAAACACCTCAACA
TGCTTCTCATTTTCAGCCAAGTACAGCAAACTCTGTTTGATATTCTCTGATTTTAACCTTGGATCAAACT
ATTTGGCAAATTGCTAATTTGAACAGGCTATTGAAAACAGACAGTGTATCTAGCAATTCATTCATTCATT
CAATACTTAATGAACAGCATTTTGGCAATTCAAAGCCTGTTCCTTGTATTGAACTATATTGGTGTATTAT
CCATATGGCCTAGAGTACATGTGTATTATTCATATTATCCATATTGGTGTATTATCCATAGGGCCTAGAG
TACGTGGCTTCTGCCCCCAAATGTCTTACAAGCATGTTAAAGTACCCAAAATCCATCCAACTGAAATAAT
TTGACCCATACATTATCAAGGTTCAAGATGTGTGATAAGCCATGAAAAGGTTGGTGAATAGCCTAGGGTG
CATATGGTGAGTGAAGATGGATGGAAGAAATTAGGAGTCCAGATTTATTGGGGAGACTTTATGAAAGCAA
GGAATCATGATTGTTGCAAAATAGACAGTGAAGAAAGAAATGGTGTGGTGGTGAAAGTCTTCCCAAAGTC
ATTTAAAGTATTGGCAACCCAGATACTTGGCAGCAGGAGTGATAATTGATGTTACAGGAATCCTATGTAA
ACTTTTGCACATTTTAATGAAGATTTTCTATAATATGCTGTCTGGTGACTTCTCCCAATCACTGTCAAGG
GCTAGACTTCATTATTTTAAGATGTCTTTCATTCATTTATTCACTCACTCATTCTTTTTTTTAAGCAAAA
TTTATTAAGACCTGTAACATACTTAGCACTGATGTAAGCTCTGATACAGTCATTCAGTAATGTCAGCCTG
AGCATTTGCAGAACCCTGAATATGGATCCTCATTTTTCCCTTGGGGTCACCCGAAGTCTTACCCCGTTCT
GTGTTTACAGTGTGAACCTTATTCTTATAAGGAATATTTTTTGTTTCTTATTGTGGATTGTCTGTACTTC
CATCAGATGTGACTCAGCTTCAGTTTTTCTGATTACCTGTGTTATTTTCCAACATGTTGCAAGTGATAAG
ATCAGTATTATCAGGTCCCTAATAGCATAACAGGTTCGTGTGAATTAGATATATTAAAATGAGAAGATTT
TTAAGTCATTTTTTAGTCAATTACGTGTATAGAAACATAGTTATACTTATCTCAGAAAGATTCCAATTCA
AAGGATAGGAAATTAGAAAAACATGTAATGTTTCTTCTGAGAACAAATATATTCAAATTTTAATGACAAA
GATATTAGGATTATTTTTCTAGTAATTTGATATACCTGATAAATTAAAATAAGAAATCATCAACCTTCAA
GCACCTTATAATATATAATCTTTGATTTAACTTTTTTGGACAAATTAAAAAATAATTCTCTAACTCAAAG
CAGGCAGAAGTATCTAATATTTATGCTTCCATCATTAGTATTTTTAAAGTACAAGAGACAAAAGCATTTT
TAATCTCATGTATATCATACATCAATATTTTTTAAGTCATCAGCATCACTATGCTAAGGAAATATTTTAT
ATAAAGAAAATTATTTTCATAAAATAAAGAAACCATCTTTCTAGAGAAAATCCACATAATCCTATGCCCA
AATATAAATATAACATTTACAGTACTGACTACAGCATCCATCTTATTCTAGTTTAACATACTTCAGTGTG
CATTTTATCATGTGCTTACCTCATCCTTTTTATAAAGTATCTCTTATCAGCTCAACCATTTCCTAATGTT
ATGCACTAATGGTAATAACATTGTAACAGGTCCGAGAATGCTGTGGCCCAAACTACTCTGATTCCTGTGT
GGCACTCACACCTGGCCCTGCATCATTTCTAGAGCATTTTAAGACCAGGTTAAAGACTGGGATCCCTTCA
TGTATGCTGATCCCAAGAATGGACCGACCTGGCTAAAGTTTATTGCATTTCAAGATATAAAACAACTTCT
ATTATATTTTTCTTTTGGTGATATTTGATTTAACCTAAAGGAAAACAAAAGAACAACTAAATATTCATTT
CTGCCTGTACTAACAGGGCAGGTAAGAGTGCCAGAGTAACAAGTAGTTTCCAAATGCACAATGAAACACA
GGAGTGCATTGGCCAAAGAGAATGCAAAATATCAGCTCTTGCTATATAGCTAACAATGTGCTGCTCTTTT
TGGAATTAGAAAATTATAGAATATATTTAATAACAATCTGGTATATGTTTTCATGTCAATGAAAAGTGGA
TAAATTTAGATGGTTGCTGTTTATGTGCATTTGATCAAATCTTTTCTGAATTTGACATGAAAATACACTT
GTGCAGCTTTCATTGGTTGGGTCACAATTTTAGAATAAAACAAACTATTTGAAAACCATTTGCAAACTAA
TGTACAAAAGCAAGATCGCAGATGATTATATGACTCTGGCAGCTTACATAAGCTTTCTGCAGGATTTTCT
TTCAGAATCTCTATACATAGGCTCAAACAGAAGTTATTTCCGTTGTTAGCACCATATTTTAAAGAAAAAA
AATACTATGGTGTTGTATCTAATCTTGTGACCCCTGACCTTTACCAAAGCGGATTGGCATTATGTTTAAG
TTCTTAAATTACAGATCAAGAAAATGCATACAGAAGATGGGGGGGGGGCACACCTAATTAATTTTTATAT
TTAGATTAAAGAAAATAATTAAATGTGTTTTTTTGTGGGATTGATTTTCAGAAGCTAAATGCAACTAGTT
CATCTGAAGGCAGCACGGTTGATATTGGAGCTCCCGCCGAGGGAGAACAGCCTGAGGTTGAACCTGAGGA
ATCCCTTGAACCTGAAGCCTGTTTTACAGAAGGTAAGCAAAACAATAACATATGTGGTCTTGAGTATCCT
CTTTTCTACCCATTTTTTCCTATTTATTTAAATGTCTGTTTATTTGTCTACCATCTATTATCTATCTATC
TGTATCTATCTATCTATCTATCTATCTAGTAATCATCTATACCTATCCAACAACTGTACATTTATTTGTT
TTTTTTTGCATTTGCTGTTTGAAAAAAAATGCAACTTTTTAAAAGGCAAAGTTTAATTTATGTAATTAGA
TATTTTCATTTTTATGAATCATTTTTAACTCTAAGAAATTATTAACTGGCTTTTCTGTGGCCTTCTAAAA
TATCTTACAGGAGAGAAAGCCAAATCACACACATCTCTCTTTAGTTTAAAAATTCAATAAATAAGAAAGT
GAGAGAAGTAATTTATTATGTACTATTTTGTGATATTATAATGGGTAATAATTGATAAGTGTACATTTAA
ATTTGTCCTTGACTGAAACAGCTCCTATTTCAGTCAAGGTCAAATATTTTTTATTATTTCTGAAAAAAGA
TAGATCATAAAAATGCCAAAATATACTATGAGTCATATGATATGGGGCAATATGTCACTGGAGTAATCGC
AAAAGGATTTTCTGAAGAAAGCTAAAATTATGTAATTTGAGGTATGGATCAGTTATATATTGTAATAGCA
ATGCTGTGTATCAAACCACCAAAAACCCTGGGCTCTAAGCTGCTTTTCTAGTTTTGACTCCTATTTCCTT
CTGTGTAACTCACAGACTTTCTTGTCACTAAGTTTTACTTGTATCATTGTTTCTCTATTCTTACAGCTTC
ATTTTCTACATATGTCTCTTATATATCCTTCAAGATCTAGTCTCAAATCCATTTCCTCCATAAAGCTCAG
AAATTAAAGTTTACCAGAAAACTCTCATAATACTTTGTTTTGTGATAATTGTTGCTTTCCATAACTATAG
AATTGTAGACAAATTGCCCCAACTTAAAATGTACATTCTTTGAGGACAAGGCTATGTTTTACATGTTATA
GTATTACAATTTGTTCTATGCAATTTTTTGACAATAGTAGATACTCAATAAGTATTTGTTGAAGAGCCTT
TGATCTAGCAATCCAGAAATTATACAAAGGTGTTTATTGGATTGTTATTGATAATGGCCAGATTTAAAGC
AAACGAAGTATTCAATAATGGTGGAATTGGGCTGGGCACAGTGGCTCACACCTGTAATCCCAGCACTTTG
GGAGGCCGAGGCAGGCGGATCACTTGAGGTCAGGAGTTCAAGACCAGCCAGGCAAACATGGTAAGACCCC
ATCTCTACCGGGCGTAGTGGTATGTGCCTGTAATACCAGCTACTTGGGAGGCTGAGGCAGGAGAATCGCT
TGAACCTGGGAGACAGAGGCTGCAGTGCATGAGCCGAGGTCAGGCCACTGCACTCCAGCCCGGACAATAG
AGTGAGACTCCCTCTCAAAAAAAAAAAAAAAAGGTGGAATAGTTATATTAATTATAGTAATCATATTTAG
AGAAATATTATGAAATCTTTCACAAATTTATTTACTTATAATAAAGATGGGAAATAGTTATACCATTAAG
TGAACTAATCAGAATTCAAATATGTAAAGTGTCCATATAGAGTGGAATTACACTCATAGGATAAGGACAG
GATGGAAATACCAACTTTTGGTAAGTTTATTTTCTTTTTGGTTCTTCTATTTTTTATATATTGTGTTTTT
GTAATGTAATCCATTATAGTAGTGCTATAAACATAAAAATAAATATTTATTAAACAAATGATTAAAAAGC
CATATAGATGATTTTAAGATAGCTTTTGTAAGCGGAAGCTATCTTAAAAATTAATGTTATTTACAATGTA
TTATCAGGTAATAATGTAAATGAATCTCCCACCAACACAAATATACCTAATCAAAGAGTAATTTTTTGTC
TTCATTTTTTTCCCACATATTTTAGACTGTGTACGGAAGTTCAAGTGTTGTCAGATAAGCATAGAAGAAG
GCAAAGGGAAACTCTGGTGGAATTTGAGGAAAACATGCTATAAGATAGTGGAGCACAATTGGTTCGAAAC
CTTCATTGTCTTCATGATTCTGCTGAGCAGTGGGGCTCTGGTAGGTGATGCATGATCCACTCCTTCACCT
TTCATCTGAAATCTTTTCCCTTTCCCTTCAATCAACTCATATTACCCACTTTTAAATTAAGGTGTTTGTA
AGAATGAGAAGAAATATGTGTGACGTGTTTAGCACATATGAGAGGCTTAGTAAATAGCAATTTTTGTCAC
TCTGTCTGGAGTAGCCCTCGGGTGGAACCAAACTCAGATCATTATGGTTTCTTATAATGTTTAAAGAAGG
ATCTTTCTGACTTTCAGTCATCAGAGGCAGTTCTTATTAAGACTGGTTATGTAGACATGATGTAGGATTA
TCAGCTAAATATCAGACTGAAGCACGATATTTCCCTGACCCCTTTGCAGGTGAGAACTAGAGTGCATGGG
TGCCGGTAGGAGCGAACTCCACTCACTCACTGCTCCACCCCTCACAGGAGGGGGAGCGCAGGTGACTGGG
TGCAGGAGCCAAGGCAAATGCATTTGGGCACTGCAAGAGTGAACTCCATACCGGCCCCACAGGAGCGTCT
AGGGGAGGGTGCCTGCGATCCTTGAAGCCCTAGAGGAAGTGTTACAGTGCCCTTTTAGCTTTGCCATCCA
TGGATGGCTTAAATGTTAACAGTTCAGTGGAGGGTCAGAGTGACAGCCTTTTGCACCCACACTTGTGGTA
CCCAAGTTCATGTCCGGCGTCCAGGAGGAATGAGTTTGTACAAATGACTTGAAGATGGTAAATACAGGGG
ATTTTATTGCCAGCGAAAGTGGCTCTCAGAGGGAAGAGGAGCTGAAAGGAGATGGAGCAGGAAGGTAATC
TTCCCCTGGAGTCTGGCCATCCCCAGCCAGACTCCTCTCCGAAGCTATGCTGTCAAGCTGTCCCTCTGAT
GTCAAGCTACTTCTCTCTAATGTCCAACTGTAGTCTCTGATGTCCAGCTGTTCCTCCTGTCTGCCTGCTG
AGTTCTGGGCTTTATATAGGCACAGGATGGGGGCAGGGTGCACCATGGGTGGTTTTGGAAAAGGCAACAT
TTAAGTGAGAAAACAGGGATGTATATTCTCACTTTGGGCCACGGTTCCAGGCTTGAGGGTGGAGCCCTCG
CCAGGTACCCGTCCTCTTCTGCCCAGAATTTCTCTGCCTCTTGTTCCTGTCAAAATTGCTTAACATAAAC
TCCATGCTGCAGGGGACTCCTCTGTCTTCTTCACACTGATTCGCTATTGCCAACCACAGTGAATGATAAG
AAGTAGACTCACTTAATTACTGACTAGCAAAAAAATGATGGCATTACAAACTTATGTCTGATTTCATTCA
ATGAAATGATCAACTGGATCAAAATATTAATATAATGAAAATGATATGACCTATTTTCTTAATTGGTGAT
ACAAATGTGGTTGCATTCCTTTTACTGTTTCAATTTAATTAATAACTAGAGTGTTTGGTGAGTTGATTTC
ATTAGGAGAATTACTGCATTGGATCTGGAGGCCTCTAAGGCGAATTCTGATTTGACTAAGAATCCTGTGT
CCTGCCATATACTCAGTTTAAAGAGGATCAGCCATGCTTTATTTTCTTTACCTTTATTATTATTATTATT
ATTTTTTAGACAGTCTTGCTGTTGCCCAGGCTGGAGTGCAGTGGTGTGATCTCGACTCACTGCAGCCTCC
ATCTCTTGGGTTCATGCCATTCTTGTGCCTTAACCTCGCAAGTAGCTGGGATTACAGGTGTGAGCTACCA
CACCTGGCTAATTTTTGTACTTTTAGTAATAGAGACTGGGTTTTGCCATCTTGGCCAGGCTGGTCTCGAA
CTCCTGGCCTCAAGAGATCTGCCCGTCTTGGCCTCTTAAAGTGCTGGAACGACGGGCGTGAACCACCGCA
CCTTGCCAGACATGCTTTCTAAAGCCAAGTAGAGAGAGAACTATGAAGTCTCATTAGTGACTAGTACCTT
TGCTGTAGGAGCTCTTTGTTCTCAGTTACACCCAGTCAGTGCTCACCAAATTGCACAACGTGCTGGCACA
GTGGCTGGCTCCTCAGGGGTTTACAGCTTCAGCTATAAGCAAAGCCCAGAAACCTTTAGGTCCTTGTATG
GAGCTCTGGTTACAAGCCCTGATTCTTGTTATCTAAAAAAGAAAATGTTCCTTTGTCTTTAATCCAGGCT
GCCAGGTTTTCCTGATAATTTTTCCGATAAGAAGATCAAGTTAGATAAATAGTCTTTTCATTCTGGAAGC
CTCAGGAGTTCCTGCAAATGAGTTACCCACTCTTTCCCAAGGGCTCTGGAAAATTCTGTCAAAGGGAATT
TCCAAACGTACACCCACCCGCCTCCACACACACACAGACACACAGAGAGAGGGAGAGAGACAAGAAAGTG
AGCAATGACAATCCTTTCCTTTTTCTGTAGGCTGAGGGACCTCCCTGCTTTATATCTGCATTACTAGAGG
ATGCATTCCATTGAGTCTGCACTGAATGAGACCAATCTACTCCCAGGCGTTCCACTGCCTCCTGATGTAG
AGAGAAGCAGCTGGCAGTCTCTCAAAAATTTTAAGCTCTTTGGGGGTACACTGAGACCAAAATTTAAAAA
TTACTGAAACCCTTGGTTGACTGAAATGCCCAGTCAGCAGTCATTTATGATCAGATAATGATAAAGTAAA
ATTCAGCCATGGGAAACATTAAACCTTCCAGCCTTAGGCACCTGATAAGAGCTTGCATCGTTTCCTTTTT
TAAGAAATCATCAATTAGAGACTGTTTCTGATCATAAAATTTAATAGAATTTTTTGACTTACAGGCCTTT
GAAGATATATACATTGAGCAGCGAAAAACCATTAAGACCATGTTAGAATATGCTGACAAGGTTTTCACTT
ACATATTCATTCTGGAAATGCTGCTAAAGTGGGTTGCATATGGTTTTCAAGTGTATTTTACCAATGCCTG
GTGCTGGCTAGACTTCCTGATTGTTGATGTGAGTATGCTGCACTTTGCTGCTTTATTCATTGGCATATAT
GTAATAGTTCTAGCAATGGTGCCTGACACAGTGTAGGCACTCAGTAACACTGTATCAGCCCAAATATAAA
TTATGTTTCTCATTTCACAGTGAGAGGATGCCTCAAAACATTTTTTACCAATTTAAATACATATACATTC
ATAGATAAAAATCAAATGCCATCATACTATACTTATTCACTTAATTTCAAATTAATATTTAAAATCTCAA
GTTATGCAAAATAAAATATGAATTTAGAAATTTTGCTTTTTGCACACTCACATTTCGCAAAATAACTTGT
ATTTAAATTTTTCACAGGCATCTTTGACATTAGTATGTTTGTCATCACTAAAGCCTGTTGAGTTTAGGTC
ACACAGATGAATCATTAATTACAAAGAAATTTGAAAGTCCAAAAAGCAAGAGACACCACTTGATTTGTAT
GATATAGAAGCAAATTGGCTATTGACCAAGTAGCCAAAGATTTTATTAAACCACATTGGTGTTGAAATAA
AATAAGATAGAGTACTAAAATATGAGGGTTTTTATATAATTGAATATGAGGCAAATCTACCATTAAATGT
ACTACTACTATTAAATGTATAAAGGTTACATGCAGAATTACATTAACAGTCTCTGGCAATAAAGGAAGAC
AATAAATAATATTTAGAACTACATAAGTGTGGACATTACAAACAATAGAAAATGCACCAAAACTATAACC
ATTCTTTTATTTGTATAATGGGATTATGCATGATACTATTTCTTTTCTCTATTTTCTGTATGTACTTATC
ATAGGTTGGTAAATCCATAATAAAAATATCTGATACTTGATATATCTATGTTAGGATAAAAGTATCAAGT
CAGCACTGCTTGAATATAAGGAAACTCTTCAGAGAAATCTAGTTGTCCTGCAGCTAATGATCATATTACC
CAAAGTACTCTGATATTTACCTTTTTAGATTTAAGAAAACTATTATGATAGTATATGAAACTGATCAACA
CTTTGCCTTAAATCAAATATGCTTATTGCTCATCTATTTCATTATGAAAGATACAAATATAAATAAGTCA
TTTTTCTAGTCCTGCAGTAGCTTACAGTTGAAAAGTGAGGACAGCTGCGTACACAGTAAGTCGACACCTG
TATTACAAGTGCCACCTCTTTACTTGAGGAAGGAGGAAAGGCTTCAATAGGGAAGTGGAGTGTGAGCTGG
AGCTTGAGAGATGTGAATGCTAGCAGGCACAGCTGAGGGAGGAACACGGATTCGTTAAAACGTTGGTGCA
TGACATGCAGGGCGGGTTCCAGAAACAAGTAGATAGGGTGAGGTAAGCCTTTGTAATGGGATGATAAGGT
AAGAAAGATAAGTTAGAAAAGATCTGAAGAACCTGAGATGCCATCCAAGGAAATTTGGACTTATTATTTA
ATACAGAGGAAGCTATTGAAGAATTACATATAGGGAAGTGACAAGACCTGCTTGTTCTTTTAGTGAGGGA
AGTTAGGTGGAGGTGAGAATGACGGAATAGAAAGGAGATTTATTTAGAGATCAAAACACCAATTAGGAGA
TTGCTGCAATGTCCCAGAAAGAGAAGGCCTATATGTATCTTCTTTTCCACATTTAGCTACACAAGTCACA
TAAAACTGAATATTTTACAACTTCTTTTCAGCCAGTAAATACTACCCCATTCAAAATATTTTCCTCTGTC
TAACTTTTATCTTTCATCCTTTAACTTATGCTTATCTCTTTTTGGTTCTGTCTTCAGAGAAGGTAAAGTA
CTACAGGTCCTTATATCTTAAATACAGAAAAGCTTCACAACTCATGATAATTCAGTAACTATTTTTCAAT
TATCTGTTAAAAAGGGACTTACAAAGCCTAAGAGTTTGGATTTTAAGGGAACTATATGAACTATGTAAGA
CATAATTTTACAACTCATTGTTTTCTGTATTCAAGAGGCTTCACTTTCAAATTGCATGTGCAAAATTATT
TTGAATAAGTTGTTTTTTGTAACAACTTTCAATGTGCTTCACTTATTTTCCTTAAAAAATATATTTTTCA
AATATATTAACACCATACTCTTAAAAGCTGTATTGCATATTTATTTTTATTTATCTGCTTTTGAAATTCA
GGTGTACTTTAGAACAAAATAGCTTATATAATTTTAATAATTTTTCTATATGTTTTCAAGGAAATTGGAC
ATGTGTATGTCCCCCGACCGTTTTTCTTTTTCTTTTTAGCTAAGACTTTATAATTTTTCTCAACTACATT
AGTCAACTGTATGAATAACTAAAGACAACATTGTTCTTGCAATTTCTAATTTATCATAAAATCTCAACTT
TTTTTATTCACTAATTTTGTCTGACCTAATTAATGATATTATGCCCTTCAAACTGAAATTTACAAAAGTC
AAAGCTGCTTTTTAGAGGCCTATTCCTTTTTAAATGTGTTCATGCTCATATTCACCAGTGGTTTGTATAG
TTTACTTGTGTATCAAATGTTACTTTCCATTTCAGATCTGCTCAATATTATTAGAAATGATACAGAAATA
AGTTTTACAGATCTGTAGAGGAAGATCACATTTCTCTCTCTTTTTTTTCTTTACTTTTAATTTTTTAAAA
ACATTTCCTACCAAGAATCTTGAAAAAGAGCACATATATGGGCTTCTTTTTTATAAGTGTTCGCAGACTA
GTATCATTAACTTCACCCTGGGAACCTGTAGAAATGCAAATTCTTAGGCCTTTCCCCAAACTTACTAAGT
CAGACTCTGCTATTGGTGTTTTTAACAAGACCCCTGGGTGATTTTGAAACTCATGAAAGTTCGAGAATTA
CTGATTCATTGCATAGAGCAAGGCTGAACTGTGTAGACATTTTTATATGTAAATAAGAAAATTGTGTTGC
TTTTTCTGTATAGGTCTCACTGGTTAGCTTAACTGCAAATGCCTTGGGTTACTCAGAACTTGGTGCCATC
AAATCCCTCAGAACACTAAGAGCTCTGAGGCCACTGAGAGCTTTGTCCCGGTTTGAAGGAATGAGGGTAA
GACTGAATGCCTTAGAGTTTGTCAGAATTATTATTGAGAGCAGACTGACACTTTGTACCATGGAAATGTC
AAATTTATGGAGAATTTGTGTCTTACACATTCATACTGACATAGCTAATCAATCAAAAATAATATTTACC
AGATGCCCATAATACTTGGCACTGCTGGAGTCACTCACAGAGTAGTATATTGCCAGAGGGATTGTTTCTG
ATTAGCTAGATTTTCACTTCTTGGAAAATCTCTATAGTTATGCTGCTGATTTGAATCAAGATTATTTATG
TTCACTTCATTTATAAATGTGCAGGAAATCCTACTCGCTGTAGTTTAAGCCTACCAAATCATTGCTCATC
ATTTCTTCACTACTCCGCTGTGATACACTTTGAGCCTTTTGATGTTTGAATCAGGCCTTTTAGTTCTTAA
ACACAGGCTGAAATGGCTAAAAAGTAGGTCAACTGGAAATCTAACGCTCATTTAGAAGGGTGGTACAAAA
GAACAGAGGAGTTTGTGCTGACATTTGTCGTCCCCTGAGGCACAAAACCTGAGACCACATACCCTCACCA
CCTAGAAAATGATGATGCCTTGTCTCAGTTGTTTTAGCTGGTTCAAAGAGGATTTTAAAAAAATGATACT
TTTTGTGATATTTGAAAATAAGTTGCTTAGACTTTATCTGCATGTTATAGTGATACTAGCTCATATTTTC
TAACTAAGAAAATAGTTACTTAGACTTTATCTAGTGTTACAATCACAACTAGAGATGAATGGTGTGTGTA
GATGTGTGTCTGTATATGCATGGTTACATAGAAAAGTGTTATTAGCGGTAAAATTCTTTTTACTTTACCA
ATTAGAAAGAACAGTTTTTGCAGTAGAAGGCTTAATAAACAAAAGGTATCAATCTTTCAGTACCAGAATA
CTGTTTATATTTTCTGTGTGGAATTTGATCCCCAAGTGGTCTCTTTTACTCTCAAATTTTGGACAGCAAA
TTGTATGGTTTGTATGATTTTTTGAAAGTGATGTTCACTTCTATATTCATGCCACTGTTTATACTCTTAA
TTATTTTTGGCATTTGCTGTTAGTTCCATCCTTTGAGGTAAATTTGCTACATGTGTGTTATTACCTCTTG
AGAAAACATTCTCCAATATAAAATTCGTTGTATACTCTTCTGATTTATAATTTTAAAATTCTTAGTTGGA
GCTACCAGAGTCTAGTTTCTACCCAATATTCAACTTTGAAACAGATTTTTTTAATCATTTGACTGTTCTT
TTAATAATGTTTAAAAATAAGTAAATATTTGTTGTTGGCTTTTCACTTATTTTTCCTTCTCATCCTGTGC
CAGGTTGTTGTAAATGCTCTTTTAGGAGCCATTCCATCTATCATGAATGTACTTCTGGTTTGTCTGATCT
TTTGGCTAATATTCAGTATCATGGGAGTGAATCTCTTTGCTGGCAAGTTTTACCATTGTATTAATTACAC
CACTGGAGAGATGTTTGATGTAAGCGTGGTCAACAACTACAGTGAGTGCAAAGCTCTCATTGAGAGCAAT
CAAACTGCCAGGTGGAAAAATGTGAAAGTAAACTTTGATAACGTAGGACTTGGATATCTGTCTCTACTTC
AAGTAGTAAGTAATCACTTTATTATTTTCCATGATGTGTAATTAAAATGAGTCTAAAGTTTTTCTTCCTC
ATAATGAGATATCCACCTGTTAGAATGGCTATTATCAAACAGATAAATGACAATAAATGCTGGCAAGAAT
GTGAAGAAAAGGGAACCCTTGTACATTGTTGGCAGGGATGTAAATTAGTATAGCTTTTATGGAAAACAGT
ATGGAGGTTTCTCAAAAAACTAAAAATAGAACCACTATGTGATCCAACAATTCCATTACTGGGTATATAT
ACAAAGGAAATTAAATCAACATGTCAAAGAGATGTCTGCACTCTCACACTCACTGCAGCACTATTCACAA
TAGCCAAAATATGGAAACAACCTAATTGTCCATCAACAGATATGTGGATAAAGAAAAGTGTGTGTGTGTG
TGTGTGTGTGTACATATATGTATATGTATATATATACACACACGTATTTCTATATACACACGTATAGATA
TACACTGTATATGTATATATCTATACACATATATAGACATACACAGAAACAGTGTTTGTGTATGTGTGCG
TGTATATAGAAGTAGTCAGGGAAGGGGCAGAGCCTGTGGCACTAAGAAACTGAGAAAATGTACAAGACTT
TTGTTTTCAGAATTACTATGTCCGCACAACAGAAAAAGTATTTCAAAAAGTAAATGCGCTTGAATGTATT
TGTTTTCAGTTTAGGAAACTGCTTCTTTTTGTAGAGTGCCTTAAAATAGTATGTTCAACAATATTAAAAA
GATTTTCAAAAATAAGCCCTCGTGATTGATGATTGGTAATAATCATTTAAAAACTTATTGGATGTATATA
TATGTGTGTGTATACACGCACACACACACACACACACCCCTATAGACATACACAATGAAATAGTATTCAG
CCTTTAAAGAAGAAGGAAATCCTGTCCTTTTATACAACATTGATTCACCTGGAGGAAATTAAGTGAAATA
AGCCAGGCACAGAAAGACAAATGACACATGATGTCACTTATATATGGAATCTAAAAAACACAAACTCACA
GAAACAGAAAGCAGAATGACAATCACCAGGGGCTGGGGGATGATGGGAGATGTTGGTCAAAGGATACAAA
ATTCAATTCGACAGGAAGAATACATTCTGTAGAGCTATTGTACAGCATGGTGACTATAGTTAATAATAAT
ATATTATATACTTGAAAATAGCTAAGTGAGTAGATATGTTTTCTCATCAGAAAAAAATAAGTATAAGAAG
CGATAATTATATATTACTTAGCTTGAGTTAGCCATTTCACAATATATATATATTTGAAAACATCATTTTG
TACAACATAAATATATTCATTTTTATTTGTCAATTAAAAAATGAATATATTTTTGAAAAGCAATTAAAAT
AAAAATGCATATACATTTTAGGAACTCTATATAGATGCACTAAAACTATATAAAAATGATATAATACTAT
ACAACAATAAAATAAAATTTTTCTTCCTCTGTGTTTACAAATACTTCCTTAGGCCCATCTGCCTAGATTC
CTCTTACCATGATTGAACTATCTTTTCTGCCCCACGCTGGAAACATGATGGTTCTAAAAACTTTATTGTC
TCCCTGACTATGCATTTGGTAGCATAGCCAAGTCCTTTGTTACTGGGAGTTTAATCTAGGCACTCATTGT
TTTCCTCCCTTCCTACTCTGAGGAAAGAAGTGCTGGCCCCAAGGGGGGTTGAAAAGGGGTGTGTGTGTGT
GTGTGTATGTGTCCACACGCGTGTGTGTAGATAGAGAAAGAGAGAGAGACTTTCAAATAGGAAAATTGCT
CTCTTGCAAATGAAAACTTTCCAATTAAGACTATTGTGTCTGCTATGCACTCATAATAATTCATTCAGCT
ATTCAACTGACTGCAGTATTAAATCTCCACTAGCTCCTGGACACAATCCACTTACACGATCCTCAAGACT
ATTAAAATAGTCAGGAAAGGGGAAGAGCCTGTGGCACTAAGGAACTGAAAAAATGTACAAGAGTTTTATT
TTCAAGATCATTATGTCAACGGAGCAGAAAACAAATATTTAAAAAAGGAAATGCAGTAGAATATATTGTT
TTCAGATTAGGAAACTGCTTCCTCTTATAGAGTAATCACCTCAAAATAGTATGATCAACAATATTAAGAA
GATTTTCAAGAATAAGCTGTCATGATTGGTGATTGGTGTAATAATCATTTAGAAAAGAATAAGTAGAAAG
GAAGCATTAAGATAAATAATGCAGCATACTTTTGAGCTTGTCTCATGCTGCTACTATACACATGAAATTT
TTTCATCAAAGTTCATGATATATTTTTATATAAACACATCAGAGTCAAAGATTGTTCATATTGTTTTTAT
GATAGCATATTGTTACAGTAGATCATTATTTAATTATATATGCTAAATATCCACATAAGATGTTATAGAG
GAATATAAATTTGAAGTATTTTCAATGCATATCGCAAAACATTGCCCCAAAAGTGAATACAAATTTCAAG
CTTATTTATATGCCTGTATTGAATACATGTCAAATAGAATTTTGATCAATTATTCAATTTATTTTCTAAA
ATTATAATTTTGGGAAAAAAGAAAATGATATGACTTTTCTTACAGGCCACGTTTAAGGGATGGATGGATA
TTATGTATGCAGCTGTTGATTCACGAAATGTAAGTCTAGTTAGAGGGAAATTGTTTAGTTTGATTAAATG
TATATTTCTACAATATTGTAATTTAGTGATATTGTCAATAAAATAAAATTATGTGCTTAATTTATAAAAC
CCATCTATATTATAAGGATAAAATATTTAATCATACTATTTCTTTCAAAATTATCATAGGATGATTTTCT
CTAATCACTCTGTATCTTTTAACATATCTTTTCTAGTATTTAGCAAGGCACCTGACACAAAACTTTATTG
TATGTATTTTCAAAATGAGACATTTTATTTTTGGCTCTGATAGTCCTGGTCATTTGTGCATTAGAAGTTC
TCACAGGCAATATTTTTTATCTGTAATATATTTCCTCCAGCTTTTGATCTTCCTTATAATAGGAAGGATA
TGACTAAAAACGGGGACAAAAATAAACAATTTAGTGTTTCTCTTGGGAAAGTGAGATTAAGTGGTAGAAG
GGAGGGACTTCCCTAATCTACTTTATACATACCAGTACTTTGAATTCTTTTCTATAATTTTCATTAATTT
CTCACTATTTAATGAGGAATGAAGTCACATTTTGAAAAAAAAAAAAAAAGAGATTGATTTCTGGTATGCC
AGAGCATGATAATAAAGCTCAAAATGCTCTTTCCCTAGCACCAGCAGCTAGCTTTCTGAGTGAAGAATTC
CTGAGGTTTTTTTTTTTCTTTTTTCCACTTCATAAAAACAGAGAGGGAGCAAGAAAGCATGAAAAGCCCT
GCATTGTATCTCTATAAGTGCTATCAGGAATTCCAGTTATGAGATTTTTCTGAATAGTAATAATAATTTA
TTGATTATCACTATTCACTGTGCCAAGGACTTTCTCACATTATCCCATTTAATCCTAAATGACAACCTTA
TTGTATAGGTGATACTAGCTCTATTTTACTACTGAAGCAAAGAGGCTTAATGCGTTAAATGGGAAAACAA
GTTTTTGAACCCTGACCACAAATAATGGCTCATACCCACTTTCCACAGTGGTTCTTACCTTTTTGATTAA
TTAATTCAATGCTCTCTCCACCTTCCTTATCAATAGCTTATATGCCATGAAACATTTTCAGTTTCTTCTT
TAATAACTTAGCAGACCTTTTCCGCTGCAAAACTCCTGGAATTTCCAGCACATTACAAAAGATGAAAGCC
AATTGAGCACTACATTTATGAAAAGTTGCTGGATCTTGAACTTTAATTAGTAAATTGCATCAGATAAATG
CAAATTTAAACCAAAATAAAACATTATCTACACACCTACCAGATTGGCAATACCAAAAAGTCTGACAATA
CCAAGTTTTACCAAGGATAAACAGCAATAAGAACACTCGTACAATGCTGATAGGAAAAAAAATAGTTAAA
TAATCCTTTAAAAACAGTTGGGTATGATCACATTATTTGAGAAAGTTAAAGATATTTTTTAATACTGCAA
TTCTACTTTGAACAACGTATCCTAAAGAAACTTATGCACATGTTTAGGATAATCTATGTACAAAAATGAA
TATAACTTTTTTTTGCACTTGCAAAAAACTGGGAGCAACTCAAAAACAGTAGAAATAGGCAAATAATTGA
ATACTATATAGTGATGAAAATGAATGAATACCGCCATATACAACCACATGGATGAGCCTTAAAAATACAA
CATTGAGTTAAAGAAACTAGACACATACTATAATTCTACTTATATAAAGTTCGAAATTGACAAAACTAAG
CTTATTGTTCAAAACTGCATACTGAGGTGTTAACTTGAAAGAAAAAGCAGGGACATCATTACCATAAAAG
TCAGGATAATGATTACCTCCAGCAGGGATGATGGAGTTTATGTTTGAGAAGGGTACACCAAGGGTTTCTG
AAGTTGTAGCAATGTCCTGGGTTATGGATTTCACTTATAAAACATATTATATTTTGCATTTATGTATTAT
GCACTTTCCTGTATGTATATTGTCTTTTAAAAATTTTAAAAATATAATTTTACATCACTGTTAACTAAAC
TCACATACACAAATAAAATCTCATCGAAGAATAGCAGTTTTACAATATTCCTGATATTTTCCATTTTGCT
GTATTTCCTTAGAAACAAAATTATGCTGGTCATAATCCTCTAAATTGATTTCATAACACAGTGGGTTATA
ACTTGCATCTATTATCATCATCAGGGATTGGTTAACTGAGTTGGTTAGAACAATGTCCTATTAGACCTGT
GAAAGCTTACAGCTAAGGCGCAAACCTACTATCACACAGTTTTCTAAACAAAAGTGGATTAGACAAGAGA
TAGTATCATTGTTACAGAAACAGTCCCTACTGAATAGGATAAAGCAATAGATTCATTTTCAGAAAGGAAA
GATCAACCTATATACCTACATGCAGACCTACTACAATGATTCTTGCCTATCTAAAGAAATGTATTATACC
AAACCCTTACACTTAGCAATTACTACTGGCCGCCACTGTTCTAAGCATATTTATATGTTAATATAGTTAA
TCTTCACAACCACACTATGAGGTTTAAGTTTGATTATTTTCATCTCACAGATGAGAAAACTGAGTCAGAG
AAAGTAAATCTTAAAAGTTTTGACATAGAATAATGTGACGCTGACATCTCTTTTGTAAGAAGAGGAAATC
TTTAATTTGCATGCTGTGTTGGGAACTTTGCTTAGAAAGGAAAGTGCATTCATAATCTGGGCATTTGTTG
GGTGAAATTGTCTATAATCATTCAGACTTCTATATGGTTATTTCATTTTCCCAGGTAATGAATAGTCTTG
CAGAACTCTTCAATAAGCATGTGAGATTTGAAGGTTCATAAAATCTGTTTAGTGTTTGGTTTATTTTCAT
TCCAGAGATTAAAACATGCTTAGATAATTAAAAACTCACTGATGTACTTTTTGTGAAACAAGTACTAGAT
ATAATGGTTACAATTCTTCATATTCTTTAGGTAGAATTACAACCCAAGTATGAAGACAACCTGTACATGT
ATCTTTATTTTGTCATCTTTATTATTTTTGGTTCATTCTTTACCTTGAATCTTTTCATTGGTGTCATCAT
AGATAACTTCAACCAACAGAAAAAGAAGATAAGTATATTAAAACTTCATCCTTGCTCTGAAATATGAACT
AAATATTTCATACTCTTTCCTTTAGCCTCCAAAATGCAATCACCAAAAAAAGAATATAAAATTCAGAAAT
TATTTTGAGACATTTGATAATCGATAAGCTTTTAAGCAATTAATAATTCAGATAGCATGTTTTTGATATT
TTTAGTCTAGAAATATGACTAATATGGCATAATTTATATATTGAATAAAGGCATCTCTATAAATACAGAT
ATTAGTAACAATAGAATGAAATGTGGGAGCCAATTTTCACATGATTACTAAGGTGGATTTTATAGCCAGC
AAAGAACACAATTTTAACAAGTGTTGCTTTCATTTCTTTACTTTGGAGGTCAAGACATTTTTATGACAGA
AGAACAGAAGAAATACTACAATGCAATGAAAAAACTGGGTTCAAAGAAACCACAAAAACCCATACCTCGA
CCTGCTGTAAGAATAACATATTTTCATTGCCTGTTAAAACTATATTACCTAACCGTTTCACAGCCCGAAT
TTCTAGAAACTAGTTATTTTTGTGGATTTGTAACACAAAGTTTTTTACCTTAACAATGGGACTAGCTAGC
CTAAATAGCTTGAAAAATGTACTTTACATATATAATATGTATAAATTATATAATGCATAACATATTTTAT
ATGTAAACATATAAAATACATAGAAATAAAATTTGCTATACTTAAGTGCCAGTGGTATCATACAAGCTGA
TGTCATTAAGACACTTCTAATAACATCAAAAATAAAATACATACATACATAATGTGAAAATATTAAATGT
TCTCAGAGTACAGAGGAGACAGATCGGAATAATTGGTACGTCACAGATTGGCCTCAGTTTTTGTCCAACT
CTGCAGATTGAATGGAATCATTAATGAAACAGGCCACAGGTTTTGCTTTTTTCTGGTTAAACAAAAAAAA
GACAAACCTCATATTTTCCCCTACTATCCCACCCTTAAATGAGATGATATCATTCTTTGTAGGGCTTTTT
ATTGGCTCTTCCAGGTGTACATTTGCCAGTGATACTGTTCGTTCAGTTTGGCTGCTGCAGGGAGTTGCTG
CCAGGAGAATCGCTAAGTTTTTCTATCACTCCTGAAGGACTAGCTCATATATTAAGTCTCAGAAAATCTT
CCCCAACGTATACGTGGTATAAAACACTTCAGTGTTTCTCAGAAATCTTGACTCTATAAATCTATTGGTG
ACAATATAAAACAGACCGTAATTAAGTGTTCAGTTGGTAAGCCGGCCAATAACTCAAAGAAAATGGATAG
CTATATTGGGTCAAACACAAAGGGTGTACAACTTGAGCCTAGTCTTTAGGAAATAATACAATTTGAATGA
ATAGAGAGAGAAGCAGAGAACATTTACTGTATGAGAAAATGTATACTTCATAGCCATATAGACAAATATA
TCAGTGCAGAATAGTGATGCATTTGAATTAGTGAGTAGTAGACACTGGTTTTCCGAGTTACATGAGACAA
GGTTACCATACGAGTCTGAAGAAATTTGTTCTAATTAAGCAATACAAATGCAATATAGTTAACAGAACAG
CCTAGTAATGTGAAAAGAAAGATTTTAGAGAGTTTAACCTAGAGACTGGTGTGGAACAATATTAGAGGCA
AAATAACCCTCGGCCATAGACAAGAAGATAAACCCTTACATACAAGAAGATAGTCCATAATCTGTGTCCA
ACCAGCAGGACTGGAACTACTCCAGGAGTGAAGTTAGCCAATAAGAAGACTCAATTGGGATGAAACACAG
GAAAAGAGGGAGGATGCAATGAAAAAACTGGGTTCAAAGAAACCACAATAACCCATACCTCGACCTGCTG
TAAGAATAGCATATTTTCATTGCCTGTTATGAAACACAGGAAAAGAGGGAGGATATGTAAATAACAGAGA
ATCTAAAATATAAGCTAGTTGATATTTTGTGAAACTGTTGGTTCCACTATCATATACTGAAGTCATATGA
AGGCACTGGGAAAAATAGTGTTAGAGCCTATGAAATGTCCAGACTGAAATAAGGATTTTAGCATTGTCAG
AACAAAATTCAATTGAGCTCTGAAACACAGATTCATTTTTGAAAAATAATTAGAATAGAGAAAAAAACAA
AATTCTCAGAATGAGGCCTTGCATACTTCATCAAGATATAGGAAGAAATAAATCAATGAAGAAATGAGCT
TGAGTTTGTTTCCATCAAATGACATGGATTTACCTGTAGTGGTAGGGGTGTGTGGAAAAAGTTCAACACA
TTCAGCTAGAATATTATCAGTGTCAATTTGGCAATTTAGCAAGTAACTAGTAAAATCCATTTATTCCTGC
ATTGACAATATGTACTATGTAGTATGCTAAGCATTTGAACTTAAATATCGAACAGTATGGAGTCTAGTTA
ATGCAACGGATAGTAATCAAATAGTCCTGCCAAAAAATGGAAGTATCCCAGAAAAAAAGGGATACTTTCA
GCTGTGAGAGCTGATTAGGGGGAAGGGGCTGATTAATCAGGGAAGTTAGGGAAGGCTTTATTAAAAAAAT
ATACTAGCTGAGGATGGAAAAAGAATAGAAAGCATCAATAGCCAGAGTGGGATGAGAAGAGCCCTGTAGA
AGGGGAATGAATTTGTGAAGGTCCTTATGTAGGAGGGCTGGTGAGACTGGAGTGCAGAAAGTCAAGGTTC
ATTTGGGACACACTGAGAATAAAGAGGTTAGGATAAGCCCAAACTTTTCTGGGCCTTGGAGGCCGTGTTA
AGGAGTAGTTTTCATCCTAAGAGCAGTAAGAAACCGTTAACGTGGACCCAGTCAGTCTGGGCTTTGTGGT
GATCACTCAATCAGTTTCACAGAGGCCGTGTGAATACATTGTAGACTTGTTTTGGAGCTATTTCAGAGAT
GGTAGGTAGCCTGAACCATAGCAATGTGCAGATTAATAAAAGTGGATGGATTTGTGAGCTATCACCAGAG
TGAAATTTAAAAGTTTGTCTATTAATTGAATATGGGAACTAAAGAAGGAACCAACAAGAATGACTGGTGT
CTTTCTGCTTTGCACAACTGGATAAATACTGATGTCATGCAGGAAATGAAGAAGGGACAGAAAGTGGTGA
GAAAATTGGAGATGCTAGTTTGCAGAATTTGGCAAACGAGTCAGAGTGAGAGAGTGAGAGGAAGGAGGAA
GGGAGAGAAATGATGAATATTTAGAAGTAGCAAAATAAAGGTTTCTTAAGATTCAGAGATTAGGTTTAAA
GGAAAGCAAAAGGAATTTTAGAGAGGAAAAGATCGAAGACAGAGGGAATAATTACGGCATAAAAATGCAC
AAGATGTGGGACAAGGACATAGTGGTCTAGGGTAGCTTTAGAAAGAAAAAGGGGCTGAGTCCTCTAATGA
ATTTGGAGTAATATATGAAAAGAACATGGAAATTAAAATAATATGAAATGCAAAAGGAAACAGAGGAGTT
TATTTAAACTGTTTAAATTTAATGTTCTAAAAAAAGTAAAAATAGAGGGCAAGAGAATGGAAATTTATGA
GAAGTTTGGAATTATCTTTGGAGCAAATGAAGGACAAAGGATTGCTAATTGTTAAATCTGAAGGGCCAAG
ATGAAGTTAGAGAACATAAATTTTTGGTGAATAAGATCTTCAGAATTATACATCTTGTTCCAGCATATTT
GACACCCTAGGATTTAAATGGGAGAACAGAACACAGAGACTCGAGACTGGAGTTGTACATTGAGATGTCT
GTCTCATTGGACAACTCTATGAACAGGGAATCTAAACAGTTTTTTATTAGTCATGGTGATATTAAAATTA
AGACCAAATTTCTGCTTTTAAGATATTTTGAACTTACTATACTCTAGGAGCCATATCTGAGAGAAAAATG
ATACTGCTCCTGCTTTTGAGGGGCCTCAAAACAAGTGGAAGGGAAAGAAAACTAAAATTGAATAAGAGCA
AACCATTTGCAATACAATGCCATACATTTTATGATCAATGAAAGCACTCAGTTTTCTGAGAGCACTAAGT
GCTTTAAACTCAAAACTGAGTTAGAATTCATGAGACAGAGAAGGAGTGGGGGACATGTGTTTTAGACAGA
ATAGTAGACAGATAAACTATGTAAAATGATACAGTAGAACCTGCCTAAGCTTCTAAGAGTGGTAGGCAGG
AAATATCAGAGGGTGGAAGTAAGGGGAAGATGCCAGACTTGGAAAGTTAAACTACAGTAAATTAATAATT
AATAAGGAAGGGTTTGAACTAAAAGTAGACACATTTATTGGGTTGAAAAAGGCCCTGAGAAGACAGATCC
AGCTGGAGTAATAGAAATCTAGTTCAGCAGGCCAAATACGCATTCAGAGAAAAGAGCAAAACAAAATAGA
ACAAGTAGTGTTTGATGTCCAAAAATCACCATTGGAAGTAATAGAAGCCTCCCAATAGAAAGAGGGCAGA
ACACTAAGATGTAGAATCCAGGCCACTAAAAGTGTCAGGATCTGGGAAAGCAAGCCATTAGGTGTATATG
TAGCAGAGTATTAGTCATTCTAGTTGAGAAGGTAGAGAAAGGCAGCCCAACAGAGGTTAAGTCAAGACCA
GATCCCTAGATTACCTGAGAAACAAAGCAGATACGTGCAAAATGGAACAATACAGAAACCAATGATCAGA
ACTGGTTTACAAGTTGGGGACTTCATTTCATAAGCAAGACATAAGGCAATTAGTACTTGGAAATAAGGTC
CAAATAGACTAGGGCAAAGATTGAATATTTCCATTGTGACTTTTTAAAAGATAATTTTATTCTTACAGAA
GAGTTACTCATAATGAATACTCTAATGAATCTATACACAGTGTCCTCTTGTTTTAACATCTTATGCAACC
ATAGATCAGTTCTCACAACTAAGAAATTAATCTTGATATAATACCATTAAATAAAATACAGAACTGAGTC
AGATTTCACCAGTTTTTCCACTGAAGCCTTTTCTCTAGAATGATGATTTTTAAAACATCTTAGCTGAACT
TTAAAATGAAATTTAAGATGCTGTAGCTTTAGTGAGAGAATATAAAGTCAGAAATCAGACGAAAAATTTA
AAAAGAGAGAGGAAAACTTGGAGAAGTATTTATTTATTAGTTGCTTAAAGTAAAATTAATACCCTCCCAA
CACATGGGATAAAAAATTTTATTACATGACAAATATTTACTAACTGTCCGTCATAACATGATGGTGTTCT
GTGCACTGAGAACATAATACGTGAGTTTATAAAACCTGGTATCAATGTGAGTATAAATAAAACAAATACA
TTTGAATACAGTTGAATATACAATATACAAAATTTTCTTCCAAGTATAAAACGAAAATAAAATACACTAC
TTTCTTTAATAGAATAGAACATTGTAATAATGTTCCATTGCATTTGACCCTCACATAAATGCTATGAGGT
AGCATTAAGAGATAAGATTTGAGGCTGGGCATGGTGGCTCATGCCTGTAATTCCAGCACTTTGGGAGGTC
GAGGTGGGCAGATCATTAGGTCAGGAGTTTGAGACCAGCCTGACCAATACGGTGAAATCCCGTCTCTACT
AAAATTACAAAAAGTAGTCGGGCATGGTGGCATGTACCTGTAATCCCAGCTACTCAGGAGGCTGAGGCAG
GAGTATCGCTTGAACCCGGGAGGCAGAGGTTGCAGTGAGCCAAGATCGTGCCACTGCACTCCAACCTGTG
CAACAGAGCGAGACTCCATCTCAAAAAATAAAAAAAATTAAAAAAAAGAGAGAGAGATAAGATTTGAGAT
CTGACATGGAGCTTCCCTATTTACACTACTTACCTGCTTTGTGACCTAAGGCAAGTTACCTCAGCTCTCC
AATCACTGGTTTTGCAAGGAATTTTTTTTTTTGTAAAATGTTGTGAGGATTAAAGATGTGTTTTTATAAA
AGCTACATTTTTTGTTGCTTTCTTAAAATCAGAAGAATTGAATTCGATTTTTTTTAAGGTTTCTAATGGA
ACTTTTACATATTATTTGTTCCAGAACAAATTCCAAGGAATGGTCTTTGATTTTGTAACCAAACAAGTCT
TTGATATCAGCATCATGATCCTCATCTGCCTTAACATGGTCACCATGATGGTGGAAACCGATGACCAGAG
TCAAGAAATGACAAACATTCTGTACTGGATTAATCTGGTGTTTATTGTTCTGTTCACTGGAGAATGTGTG
CTGAAACTGATCTCTCTTCGTTACTACTATTTCACTATTGGATGGAATATTTTTGATTTTGTGGTGGTCA
TTCTCTCCATTGTAGGTAAGAAGAGGTGCTTTTATTCAGTTAAGGAATATAGTGGTAAAAATATGTGTTT
TAAAACTTTAGAGGTGTTTTTCACTAATCTTTCTCATTCATCCCAAACTCCCAAATAAAAATCTAATAGT
CCATTGTTTTAGTTTTAGTTTGCCATTTCTCTAATTGCATGCTGTGCTTGAAATGATGAGTGGAATACAA
GGAATTTATATTTTCAGCTTTCATTTATTCTCATTTAATATTTTCATCTGTTCTCATCTCAGAAGACAAT
AACTGCAACTTTGGTAGAATAGTCTTGTACCTGGTCATACTCCTGTGGTATTGACAGTTACTGCTTTGAA
TAAACAATCAATCCACACACATATATACATAAATCATTTGAAGTAGTCACATAATTCATAAATATGACCT
CTTAAATAATTGGAATAGTGTATATGTGCAGTTATATATATAATAACACATATATAAGTTTCATGTTATC
TTTGGGTGCAGACAGTTTTCTGTGGTTTGCAATATCTCTTTTTGGAAGCAGATAGTTTGTTTGAAAATCC
AAAACAGATTTGTTATCATCAATGATACATTAATGTTAGGATACATACATACATTAAGTCCTAGGAATGC
AAAAGATTTATTGGAAAAAATATATATATACAGTGTTTATGTATAAGATATTAAATGAGGTACTGGAAGT
AAATATAAGAAGATTTAAGAGAAGGTTCTACCTATTTGGGGAAACAGAACATTCACATGGAGGGGAAAAT
TATATAGCACTCTTTAAACTACTTTCTTTAGTCGAATAGAACATTGTAACAATGTTCCACTGCATTTGAT
TCTCACATAAGTGCTATGAGGTAGCATTAAGAGGTAAGATTTGAGATCTGACCTGGAGCTTCCCTATTTA
CACTACTTACCTTCTCAGTGACCTAAGAGAAGTTACCTCAGCTCTCCAATCTCTGGTTTTGCAAGGAATT
TTTCTGTAAAATGTTATTGTGAGGATTAAATCAGATTATGTATATATATGCACTTAGCACTGTGCCTAGC
ATGAAGAAAAGACTTAGTAAATGTTCAGTTTGACCACAAGAAAAAGTTGATATTATCACCATTTACTCAT
GCATAAAAGCAAGTGCCAGGATTCAGTCCCAAGTACATCTGTCTCCAAAGCCTATGTTTTCTTCTGTACA
TCACGCTGCCTACTCCCAAATAACATAGAATCTCAGAAAGTAAAGAACTCTCATATTCCTGACCCAAAAT
CATACACCTTTAGTTCTTATGCAAATACTAGAACTAGTATTTTGGACATATAAATTAATTTCTGTACTTG
GCCACTGTATGCTTCATGATGTCTTTGGACCTTCCAGGGTTGAGTCATTTTTTTGATAGATGCTTTCCTT
GAACTAGGAAAAATGGCCCTTATTATCTTCATTTAATATAAAGATGTAAATGTTATAACACCAAACATAC
CAGTTTCATTTTGCTCAACAAACATTGCAGATTATTTGCATATATACATGTACCTAACTGTCCTGTTCAC
ATTTTGTAAAACTAATGTACTTATGTAAACTTTCATTTGCTACTATTAAGTATAACAATATTTTTGTTAT
TTGTTGATTTTCTACAGGAATGTTTCTGGCTGAACTGATAGAAAAGTATTTTGTGTCCCCTACCCTGTTC
CGAGTGATCCGTCTTGCCAGGATTGGCCGAATCCTACGTCTGATCAAAGGAGCAAAGGGGATCCGCACGC
TGCTCTTTGCTTTGATGATGTCCCTTCCTGCGTTGTTTAACATCGGCCTCCTTCTTTTCCTGGTCATGTT
CATCTACGCCATCTTTGGGATGTCCAATTTTGCCTATGTTAAGAGGGAAGTTGGGATCGATGACATGTTC
AACTTTGAGACCTTTGGCAACAGCATGATCTGCCTGTTCCAAATTACAACCTCTGCTGGCTGGGATGGAT
TGCTAGCACCTATTCTTAATAGTGGACCTCCAGACTGTGACCCTGACAAAGATCACCCTGGAAGCTCAGT
TAAAGGAGACTGTGGGAACCCATCTGTTGGGATTTTCTTTTTTGTCAGTTACATCATCATATCCTTCCTG
GTTGTGGTGAACATGTACATCGCGGTCATCCTGGAGAACTTCAGTGTTGCTACTGAAGAAAGTGCAGAGC
CTCTGAGTGAGGATGACTTTGAGATGTTCTATGAGGTTTGGGAGAAGTTTGATCCCGATGCGACCCAGTT
TATAGAGTTTGCCAAACTTTCTGATTTTGCAGATGCCCTGGATCCTCCTCTTCTCATAGCAAAACCCAAC
AAAGTCCAGCTCATTGCCATGGATCTGCCCATGGTGAGTGGTGACCGGATCCACTGTCTTGACATCTTAT
TTGCTTTTACAAAGCGTGTTTTGGGTGAGAGTGGAGAGATGGATGCCCTTCGAATACAGATGGAAGAGCG
ATTCATGGCATCAAACCCCTCCAAAGTCTCTTATGAGCCCATTACGACCACGTTGAAACGCAAACAAGAG
GAGGTGTCTGCTATTATTATCCAGAGGGCTTACAGACGCTACCTCTTGAAGCAAAAAGTTAAAAAGGTAT
CAAGTATATACAAGAAAGACAAAGGCAAAGAATGTGATGGAACACCCATCAAAGAAGATACTCTCATTGA
TAAACTGAATGAGAATTCAACTCCAGAGAAAACCGATATGACGCCTTCCACCACGTCTCCACCCTCGTAT
GATAGTGTGACCAAACCAGAAAAAGAAAAATTTGAAAAAGACAAATCAGAAAAGGAAGACAAAGGGAAAG
ATATCAGGGAAAGTAAAAAGTAAAAAGAAACCAAGAATTTTCCATTTTGTGATCAATTGTTTACAGCCCG
TGATGGTGATGTGTTTGTGTCAACAGGACTCCCACAGGAGGTCTATGCCAAACTGACTGTTTTTACAAAT
GTATACTTAAGGTCAGTGCCTATAACAAGACAGAGACCTCTGGTCAGCAAACTGGAACTCAGTAAACTGG
AGAAATAGTATCGATGGGAGGTTTCTATTTTCACAACCAGCTGACACTGCTGAAGAGCAGAGGCGTAATG
GCTACTCAGACGATAGGAACCAATTTAAAGGGGGGAGGGAAGTTAAATTTTTATGTAAATTCAACATGTG
ACACTTGATAATAGTAATTGTCACCAGTGTTTATGTTTTAACTGCCACACCTGCCATATTTTTACAAAAC
GTGTGCTGTGAATTTATCACTTTTCTTTTTAATTCACAGGTTGTTTACTATTATATGTGACTATTTTTGT
AAATGGGTTTGTGTTTGGGGAGAGGGATTAAAGGGAGGGAATTCTACATTTCTCTATTGTATTGTATAAC
TGGATATATTTTAAATGGAGGCATGCTGCAATTCTCATTCACACATAAAAAAATCACATCACAAAAGGGA
AGAGTTTACTTCTTGTTTCAGGATGTTTTTAGATTTTTGAGGTGCTTAAATAGCTATTCGTATTTTTAAG
GTGTCTCATCCAGAAAAAATTTAATGTGCCTGTAAATGTTCCATAGAATCACAAGCATTAAAGAGTTGTT
TTATTTTTACATAACCCATTAAATGTACATGTATATATGTATATATGTATATGTGCGTGTATATACATAT
ATATGTATACACACATGCACACACAGAGATATACACATACCATTACATTGTCATTCACAGTCCCAGCAGC
ATGACTATCACATTTTTGATAAGTGTCCTTTGGCATAAAATAAAAATATCCTATCAGTCCTTTCTAAGAA
GCCTGAATTGACCAAAAAACATCCCCACCACCACTTTATAAAGTTGATTCTGCTTTATCCTGCAGTATTG
TTTAGCCATCTTCTGCTCTTGGTAAGGTTGACATAGTATATGTCAATTTAAAAAATAAAAGTCTGCTTTG
TAAATAGTAATTTTACCCAGTGGTGCATGTTTGAGCAAACAAAAATGATGATTTAAGCACACTACTTATT
GCATCAAATATGTACCACAGTAAGTATAGTTTGCAAGCTTTCAACAGGTAATATGATGTAATTGGTTCCA
TTATAGTTTGAAGCTGTCACTGCTGCATGTTTATCTTGCCTATGCTGCTGTATCTTATTCCTTCCACTGT
TCAGAAGTCTAATATGGGAAGCCATATATCAGTGGTAAAGTGAAGCAAATTGTTCTACCAAGACCTCATT
CTTCATGTCATTAAGCAATAGGTTGCAGCAAACAAGGAAGAGCTTCTTGCTTTTTATTCTTCCAACCTTA
ATTGAACACTCAATGATGAAAAGCCCGACTGTACAAACATGTTGCAAGCTGCTTAAATCTGTTTAAAATA
TATGGTTAGAGTTTTCTAAGAAAATATAAATACTGTAAAAAGTTCATTTTATTTTATTTTTCAGCCTTTT
GTACGTAAAATGAGAAATTAAAAGTATCTTCAGGTGGATGTCACAGTCACTATTGTTAGTTTCTGTTCCT
AGCACTTTTAAATTGAAGCACTTCACAAAATAAGAAGCAAGGACTAGGATGCAGTGTAGGTTTCTGCTTT
TTTATTAGTACTGTAAACTTGCACACATTTCAATGTGAAACAAATCTCAAACTGAGTTCAATGTTTATTT
GCTTTCAATAGTAATGCCTTATCATTGAAAGAGGCTTAAAGAAAAAAAAAATCAGCTGATACTCTTGGCA
TTGCTTGAATCCAATGTTTCCACCTAGTCTTTTTATTCAGTAATCATCAGTCTTTTCCAATGTTTGTTTA
CACAGATAGATCTTATTGACCCATATGGCACTAGAACTGTATCAGATATAATATGGGATCCCAGCTTTTT
TTCCTCTCCCACAAAACCAGGTAGTGAAGTTATATTACCAGTTACAGCAAAATACTTTGTGTTTCACAAG
CAACAATAAATGTAGATTCTTTATACTGAAGCTATTGACTTGTAGTGTGTTGGTGAAATGCATGCAGGAA
AATGCTGTTACCATAAAGAACGGTAAACCACATTACAATCAAGCCAAAAGAATAAAGGTTTCGCTTTTGT
TTTTGTATTTAATTGTTGTCTTTGTTTCTATCTTTGAAATGCCATTTAAAGGTAGATTTCTATCATGTAA
AAATAATCTATCTGAAAAACAAATGTAAAGAACACACATTAATTACTATAATTCATCTTTCAATTTTTTC
ATGGAATGGAAGTTAATTAAGAAGAGTGTATTGGATAACTACTTTAATATTGGCCAAAAAGCTAGATATG
GCATCAGGTAGACTAGTGGAAAGTTACAAAAATTAATAAAAAATTGACTAACATTTTAAGTTGTGCATCT
TTTCTCCTTCCTGTCCACCTATTGTTCTTTTTTTCACTTTTCCATTTCAATTTCTTCCTTATGTATTCTT
GATCTACTTTTCTTTATATCCTTCTATCCTTTCCTTGCGCTCTCAGTATTTTTCATTTAGGATATTCTCC
TTGTTTCTTTTCTGTTCACCAAATGTCTTGTTTATTACAGCCTATAGATCACTTAGATTTAGATCCCTAA
AATTTGCTGTCACTCTGTAAAGTGCACATAGTATCTGCGTATTTATTTTTACAATTTTCTTTACAAAAAT
TCTAAGAAAGCTTAGACTGCAACTTTTCATAATATATAAACAAAATTCCAAAATAGCACAGATTTGACTT
TCTCATTTAATTATGAACAGTTTGGGAAATGTTTCTCAATTCTGAATGCATATTAGAACTACCTGAGAAG
CAAAAAAAAAAAAAAAAAAAAAGCCAGTGCATGGGCCAAGACCAGGACCAGACCAATTGAACTCATATCT
CTGGGTGAGACTTAAGCATTTAATTTTTTGAAGGCTCGTCATTGATACTGATTCATACCAAGGACTGAGA
ACTAGCAAATTTGGGTCTCCTTTGCCCAATAGGAAAACATATTCTCCGGAAAGTCCAAACTGTCCTTCAT
TACATAACTTATCATGTACAGAATAAAGTTCCTAAGAGGATGGCTTTCGGAGTATTTATATTGCAGATTG
AGGGATTGACATTGATCTAATTATAATAGAGGTTCTTCTTTAGGGCTGTAGTTGGTATAAAGGGAATTCT
ATTAGAAAAGTTTAGGGAGACTGTGTTCAGGTAACACATGCACACACACACACCCCATATGGCACCTTAA
CTATCCCTAGTGTATACTGGGTGGAATGAATGTGGGATGGTCTAGGAAACAGTATAAATTTATAAAATAG
TTCCCTCCAGTGAATGCTACATGCCCTGTTGCATGAGAACCTTTAATCTGGACCAATAGAAGGCAGTTCA
TGGTGGCCAGTCAGAAAGCCATTCAATTTGTCATTACCAACTCACAAATCTAGACAGGAAAGATTCTCAT
TCTCATCTTCTCCATTCTTGGCGCATCTCACACAATCCCTTGTTTTTCTGAGCTCTGTAACATACAGACC
TATTTACAAACAGAAGGGAGGAGACAAGAGAGGTAAGGGGAAGTAAAGAAAGAGGAGAAAGGAGGAGAGG
GAACGATAGGGGAAAGTAGGAGAAAGGAGGGGAGTGTAAGAAATGGGATGTTTCATGAATTATAATTCTG
AAAAAATGTCTGGTGTGGATACCTCCAGCAATGGTGAGGGAAGGAGGAAGAAATGTTGCTTAGTTGTCAA
TATGTTAGATCTAAAATATAAACATGTTTCTTCTCTTATTTATTTTTATTTGTATTTATTTATTTATTTA
TTTTTGAGATAAGGTCTTGCTCTGTCACCCAGGCTGAAGAGCAGTGGTGTGATCATGGCTCACTGCAGCC
TTAACCTTCTGGACTCAAGTGATCTTCCCACCTCAGCCTCCCGAGTATCTGGGACTACAGGCATGTGCTC
CCACACCCAGCTCATTTTTTTTTTTTTTTTTGTATTTTTTGTAGAGACAGGGTCTCCCCATGTTGCCTGG
GTTGGTCTCAGATTTCTGGACTGAAGCAATCATCCTGCCTCAGCTTTCCAAAGTGCTAGAATTACAGGCA
TGAGCCATTGCACCTGCCCCTCTCATTTTTTTAAAGAAAAGATACCTGTGAAAAAAGTGAAGAGGACATT
ATACAAGAAAATGTTACAATGATTCCATGGATTCAGGAATATTTTTTTTTCTTTTACCTGAACTAAATAC
ATATTTGAGTGTCTACTATATATCAACTTTGCAATAGCAGCTAAGAGATCAAAGAGGAATATAACTATCC
AAAGAAAGGCTATAACCAGAGGTGAAGAACCTGGAAAGCCATGCTAGAGTTTGGAATTGATTCTGTGGCA
AAAAGGAGTGATTACTCCTGAGAAGTAGA
SEQ ID NO: 3; SCN2A isoform 1 protein (NP_001035232.1):
MAQSVLVPPGPDSFRFFTRESLAAIEQRIAEEKAKRPKQERKDEDDENGPKPNSDLEAGKSLPFIYGDIP
PEMVSVPLEDLDPYYINKKTFIVLNKGKAISRFSATPALYILTPFNPIRKLAIKILVHSLFNMLIMCTIL
TNCVFMTMSNPPDWTKNVEYTFTGIYTFESLIKILARGFCLEDFTFLRDPWNWLDFTVITFAYVTEFVDL
GNVSALRTFRVLRALKTISVIPGLKTIVGALIQSVKKLSDVMILTVFCLSVFALIGLQLFMGNLRNKCLQ
WPPDNSSFEINITSFFNNSLDGNGTTFNRTVSIFNWDEYIEDKSHFYFLEGQNDALLCGNSSDAGQCPEG
YICVKAGRNPNYGYTSFDTFSWAFLSLFRLMTQDFWENLYQLTLRAAGKTYMIFFVLVIFLGSFYLINLI
LAVVAMAYEEQNQATLEEAEQKEAEFQQMLEQLKKQQEEAQAAAAAASAESRDFSGAGGIGVFSESSSVA
SKLSSKSEKELKNRRKKKKQKEQSGEEEKNDRVRKSESEDSIRRKGFRFSLEGSRLTYEKRFSSPHQSLL
SIRGSLFSPRRNSRASLFSFRGRAKDIGSENDFADDEHSTFEDNDSRRDSLFVPHRHGERRHSNVSQASR
ASRVLPILPMNGKMHSAVDCNGVVSLVGGPSTLTSAGQLLPEGTTTETEIRKRRSSSYHVSMDLLEDPTS
RQRAMSIASILTNTMEELEESRQKCPPCWYKFANMCLIWDCCKPWLKVKHLVNLVVMDPFVDLAITICIV
LNTLFMAMEHYPMTEQFSSVLSVGNLVFTGIFTAEMFLKIIAMDPYYYFQEGWNIFDGFIVSLSLMELGL
ANVEGLSVLRSFRLLRVFKLAKSWPTLNMLIKIIGNSVGALGNLTLVLAIIVFIFAVVGMQLFGKSYKEC
VCKISNDCELPRWHMHDFFHSFLIVFRVLCGEWIETMWDCMEVAGQTMCLTVFMMVMVIGNLVVLNLFLA
LLLSSFSSDNLAATDDDNEMNNLQIAVGRMQKGIDFVKRKIREFIQKAFVRKQKALDEIKPLEDLNNKKD
SCISNHTTIEIGKDLNYLKDGNGTTSGIGSSVEKYVVDESDYMSFINNPSLTVTVPIAVGESDFENLNTE
EFSSESDMEESKEKLNATSSSEGSTVDIGAPAEGEQPEVEPEESLEPEACFTEDCVRKFKCCQISIEEGK
GKLWWNLRKTCYKIVEHNWFETFIVFMILLSSGALAFEDIYIEQRKTIKTMLEYADKVFTYIFILEMLLK
WVAYGFQVYFTNAWCWLDFLIVDVSLVSLTANALGYSELGAIKSLRTLRALRPLRALSRFEGMRVVVNAL
LGAIPSIMNVLLVCLIFWLIFSIMGVNLFAGKFYHCINYTTGEMFDVSVVNNYSECKALIESNQTARWKN
VKVNFDNVGLGYLSLLQVATFKGWMDIMYAAVDSRNVELQPKYEDNLYMYLYFVIFIIFGSFFTLNLFIG
VIIDNFNQQKKKFGGQDIFMTEEQKKYYNAMKKLGSKKPQKPIPRPANKFQGMVFDFVTKQVFDISIMIL
ICLNMVTMMVETDDQSQEMTNILYWINLVFIVLFTGECVLKLISLRYYYFTIGWNIFDFVVVILSIVGMF
LAELIEKYFVSPTLFRVIRLARIGRILRLIKGAKGIRTLLFALMMSLPALFNIGLLLFLVMFIYAIFGMS
NFAYVKREVGIDDMFNFETFGNSMICLFQITTSAGWDGLLAPILNSGPPDCDPDKDHPGSSVKGDCGNPS
VGIFFFVSYIIISFLVVVNMYIAVILENFSVATEESAEPLSEDDFEMFYEVWEKFDPDATQFIEFAKLSD
FADALDPPLLIAKPNKVQLIAMDLPMVSGDRIHCLDILFAFTKRVLGESGEMDALRIQMEERFMASNPSK
VSYEPITTTLKRKQEEVSAIIIQRAYRRYLLKQKVKKVSSIYKKDKGKECDGTPIKEDTLIDKLNENSTP
EKTDMTPSTTSPPSYDSVTKPEKEKFEKDKSEKEDKGKDIRESKK
SEQ ID NO: 4; SCN2A transcript variant 3 (NM_001040143.2):
AACAGACATTGGGTACCATCGAATGACTGTCAGAACAGAAAGCTAAGGCAAAGGAGGGAGGATGCTGTGG
TCATCCTTTCTTGTTTTTTTCTTCTTTAATGAGGATAGAGCACATGTGAGATTTTACTTTCTACTCCAGT
AAAAATTCTGAAGAATTGCATTGGAGACTGTTATATTCAACACATACGTGGATTCTGTGTTATGATTTAC
ATTTTTCTTTATTTCAGGGGTTTTTCTCCCTTTGCTTGACACTTCTCTGTCCTGACACCTTGAGAAGAAG
GATGTGTTTGCTTACCCTTCCGCCATGATTGTAAATTTCCTGAGGCCTTCCCAGCCATGCAGCACTCACT
TTCTTATGCAAGGAGCTAAACAGTGATTAAAGGAGCAGGATGAAAAGATGGCACAGTCAGTGCTGGTACC
GCCAGGACCTGACAGCTTCCGCTTCTTTACCAGGGAATCCCTTGCTGCTATTGAACAACGCATTGCAGAA
GAGAAAGCTAAGAGACCCAAACAGGAACGCAAGGATGAGGATGATGAAAATGGCCCAAAGCCAAACAGTG
ACTTGGAAGCAGGAAAATCTCTTCCATTTATTTATGGAGACATTCCTCCAGAGATGGTGTCAGTGCCCCT
GGAGGATCTGGACCCCTACTATATCAATAAGAAAACGTTTATAGTATTGAATAAAGGGAAAGCAATCTCT
CGATTCAGTGCCACCCCTGCCCTTTACATTTTAACTCCCTTCAACCCTATTAGAAAATTAGCTATTAAGA
TTTTGGTACATTCTTTATTCAATATGCTCATTATGTGCACGATTCTTACCAACTGTGTATTTATGACCAT
GAGTAACCCTCCAGACTGGACAAAGAATGTGGAGTATACCTTTACAGGAATTTATACTTTTGAATCACTT
ATTAAAATACTTGCAAGGGGCTTTTGTTTAGAAGATTTCACATTTTTACGGGATCCATGGAATTGGTTGG
ATTTCACAGTCATTACTTTTGCGTATGTAACAGAATTTGTAAACCTAGGCAATGTTTCAGCTCTTCGAAC
TTTCAGAGTCTTGAGAGCTTTGAAAACTATTTCTGTAATTCCAGGCCTGAAGACCATTGTGGGGGCCCTG
ATCCAGTCAGTGAAGAAGCTTTCTGATGTCATGATCTTGACTGTGTTCTGTCTAAGCGTGTTTGCGCTAA
TAGGATTGCAGTTGTTCATGGGCAACCTACGAAATAAATGTTTGCAATGGCCTCCAGATAATTCTTCCTT
TGAAATAAATATCACTTCCTTCTTTAACAATTCATTGGATGGGAATGGTACTACTTTCAATAGGACAGTG
AGCATATTTAACTGGGATGAATATATTGAGGATAAAAGTCACTTTTATTTTTTAGAGGGGCAAAATGATG
CTCTGCTTTGTGGCAACAGCTCAGATGCAGGCCAGTGTCCTGAAGGATACATCTGTGTGAAGGCTGGTAG
AAACCCCAACTATGGCTACACGAGCTTTGACACCTTTAGTTGGGCCTTTTTGTCCTTATTTCGTCTCATG
ACTCAAGACTTCTGGGAAAACCTTTATCAACTGACACTACGTGCTGCTGGGAAAACGTACATGATATTTT
TTGTGCTGGTCATTTTCTTGGGCTCATTCTATCTAATAAATTTGATCTTGGCTGTGGTGGCCATGGCCTA
TGAGGAACAGAATCAGGCCACATTGGAAGAGGCTGAACAGAAGGAAGCTGAATTTCAGCAGATGCTCGAA
CAGTTGAAAAAGCAACAAGAAGAAGCTCAGGCGGCAGCTGCAGCCGCATCTGCTGAATCAAGAGACTTCA
GTGGTGCTGGTGGGATAGGAGTTTTTTCAGAGAGTTCTTCAGTAGCATCTAAGTTGAGCTCCAAAAGTGA
AAAAGAGCTGAAAAACAGAAGAAAGAAAAAGAAACAGAAAGAACAGTCTGGAGAAGAAGAGAAAAATGAC
AGAGTCCGAAAATCGGAATCTGAAGACAGCATAAGAAGAAAAGGTTTCCGTTTTTCCTTGGAAGGAAGTA
GGCTGACATATGAAAAGAGATTTTCTTCTCCACACCAGTCCTTACTGAGCATCCGTGGCTCCCTTTTCTC
TCCAAGACGCAACAGTAGGGCGAGCCTTTTCAGCTTCAGAGGTCGAGCAAAGGACATTGGCTCTGAGAAT
GACTTTGCTGATGATGAGCACAGCACCTTTGAGGACAATGACAGCCGAAGAGACTCTCTGTTCGTGCCGC
ACAGACATGGAGAACGGCGCCACAGCAATGTCAGCCAGGCCAGCCGTGCCTCCAGGGTGCTCCCCATCCT
GCCCATGAATGGGAAGATGCATAGCGCTGTGGACTGCAATGGTGTGGTCTCCCTGGTCGGGGGCCCTTCT
ACCCTCACATCTGCTGGGCAGCTCCTACCAGAGGGCACAACTACTGAAACAGAAATAAGAAAGAGACGGT
CCAGTTCTTATCATGTTTCCATGGATTTATTGGAAGATCCTACATCAAGGCAAAGAGCAATGAGTATAGC
CAGTATTTTGACCAACACCATGGAAGAACTTGAAGAATCCAGACAGAAATGCCCACCATGCTGGTATAAA
TTTGCTAATATGTGTTTGATTTGGGACTGTTGTAAACCATGGTTAAAGGTGAAACACCTTGTCAACCTGG
TTGTAATGGACCCATTTGTTGACCTGGCCATCACCATCTGCATTGTCTTAAATACACTCTTCATGGCTAT
GGAGCACTATCCCATGACGGAGCAGTTCAGCAGTGTACTGTCTGTTGGAAACCTGGTCTTCACAGGGATC
TTCACAGCAGAAATGTTTCTCAAGATAATTGCCATGGATCCATATTATTACTTTCAAGAAGGCTGGAATA
TTTTTGATGGTTTTATTGTGAGCCTTAGTTTAATGGAACTTGGTTTGGCAAATGTGGAAGGATTGTCAGT
TCTCCGATCATTCCGGCTGCTCCGAGTTTTCAAGTTGGCAAAATCTTGGCCAACTCTAAATATGCTAATT
AAGATCATTGGCAATTCTGTGGGGGCTCTAGGAAACCTCACCTTGGTATTGGCCATCATCGTCTTCATTT
TTGCTGTGGTCGGCATGCAGCTCTTTGGTAAGAGCTACAAAGAATGTGTCTGCAAGATTTCCAATGATTG
TGAACTCCCACGCTGGCACATGCATGACTTTTTCCACTCCTTCCTGATCGTGTTCCGCGTGCTGTGTGGA
GAGTGGATAGAGACCATGTGGGACTGTATGGAGGTCGCTGGCCAAACCATGTGCCTTACTGTCTTCATGA
TGGTCATGGTGATTGGAAATCTAGTGGTTCTGAACCTCTTCTTGGCCTTGCTTTTGAGTTCCTTCAGTTC
TGACAATCTTGCTGCCACTGATGATGATAACGAAATGAATAATCTCCAGATTGCTGTGGGAAGGATGCAG
AAAGGAATCGATTTTGTTAAAAGAAAAATACGTGAATTTATTCAGAAAGCCTTTGTTAGGAAGCAGAAAG
CTTTAGATGAAATTAAACCGCTTGAAGATCTAAATAATAAAAAAGACAGCTGTATTTCCAACCATACCAC
CATAGAAATAGGCAAAGACCTCAATTATCTCAAAGACGGAAATGGAACTACTAGTGGCATAGGCAGCAGT
GTAGAAAAATATGTCGTGGATGAAAGTGATTACATGTCATTTATAAACAACCCTAGCCTCACTGTGACAG
TACCAATTGCTGTTGGAGAATCTGACTTTGAAAATTTAAATACTGAAGAATTCAGCAGCGAGTCAGATAT
GGAGGAAAGCAAAGAGAAGCTAAATGCAACTAGTTCATCTGAAGGCAGCACGGTTGATATTGGAGCTCCC
GCCGAGGGAGAACAGCCTGAGGTTGAACCTGAGGAATCCCTTGAACCTGAAGCCTGTTTTACAGAAGACT
GTGTACGGAAGTTCAAGTGTTGTCAGATAAGCATAGAAGAAGGCAAAGGGAAACTCTGGTGGAATTTGAG
GAAAACATGCTATAAGATAGTGGAGCACAATTGGTTCGAAACCTTCATTGTCTTCATGATTCTGCTGAGC
AGTGGGGCTCTGGCCTTTGAAGATATATACATTGAGCAGCGAAAAACCATTAAGACCATGTTAGAATATG
CTGACAAGGTTTTCACTTACATATTCATTCTGGAAATGCTGCTAAAGTGGGTTGCATATGGTTTTCAAGT
GTATTTTACCAATGCCTGGTGCTGGCTAGACTTCCTGATTGTTGATGTCTCACTGGTTAGCTTAACTGCA
AATGCCTTGGGTTACTCAGAACTTGGTGCCATCAAATCCCTCAGAACACTAAGAGCTCTGAGGCCACTGA
GAGCTTTGTCCCGGTTTGAAGGAATGAGGGTTGTTGTAAATGCTCTTTTAGGAGCCATTCCATCTATCAT
GAATGTACTTCTGGTTTGTCTGATCTTTTGGCTAATATTCAGTATCATGGGAGTGAATCTCTTTGCTGGC
AAGTTTTACCATTGTATTAATTACACCACTGGAGAGATGTTTGATGTAAGCGTGGTCAACAACTACAGTG
AGTGCAAAGCTCTCATTGAGAGCAATCAAACTGCCAGGTGGAAAAATGTGAAAGTAAACTTTGATAACGT
AGGACTTGGATATCTGTCTCTACTTCAAGTAGCCACGTTTAAGGGATGGATGGATATTATGTATGCAGCT
GTTGATTCACGAAATGTAGAATTACAACCCAAGTATGAAGACAACCTGTACATGTATCTTTATTTTGTCA
TCTTTATTATTTTTGGTTCATTCTTTACCTTGAATCTTTTCATTGGTGTCATCATAGATAACTTCAACCA
ACAGAAAAAGAAGTTTGGAGGTCAAGACATTTTTATGACAGAAGAACAGAAGAAATACTACAATGCAATG
AAAAAACTGGGTTCAAAGAAACCACAAAAACCCATACCTCGACCTGCTAACAAATTCCAAGGAATGGTCT
TTGATTTTGTAACCAAACAAGTCTTTGATATCAGCATCATGATCCTCATCTGCCTTAACATGGTCACCAT
GATGGTGGAAACCGATGACCAGAGTCAAGAAATGACAAACATTCTGTACTGGATTAATCTGGTGTTTATT
GTTCTGTTCACTGGAGAATGTGTGCTGAAACTGATCTCTCTTCGTTACTACTATTTCACTATTGGATGGA
ATATTTTTGATTTTGTGGTGGTCATTCTCTCCATTGTAGGAATGTTTCTGGCTGAACTGATAGAAAAGTA
TTTTGTGTCCCCTACCCTGTTCCGAGTGATCCGTCTTGCCAGGATTGGCCGAATCCTACGTCTGATCAAA
GGAGCAAAGGGGATCCGCACGCTGCTCTTTGCTTTGATGATGTCCCTTCCTGCGTTGTTTAACATCGGCC
TCCTTCTTTTCCTGGTCATGTTCATCTACGCCATCTTTGGGATGTCCAATTTTGCCTATGTTAAGAGGGA
AGTTGGGATCGATGACATGTTCAACTTTGAGACCTTTGGCAACAGCATGATCTGCCTGTTCCAAATTACA
ACCTCTGCTGGCTGGGATGGATTGCTAGCACCTATTCTTAATAGTGGACCTCCAGACTGTGACCCTGACA
AAGATCACCCTGGAAGCTCAGTTAAAGGAGACTGTGGGAACCCATCTGTTGGGATTTTCTTTTTTGTCAG
TTACATCATCATATCCTTCCTGGTTGTGGTGAACATGTACATCGCGGTCATCCTGGAGAACTTCAGTGTT
GCTACTGAAGAAAGTGCAGAGCCTCTGAGTGAGGATGACTTTGAGATGTTCTATGAGGTTTGGGAGAAGT
TTGATCCCGATGCGACCCAGTTTATAGAGTTTGCCAAACTTTCTGATTTTGCAGATGCCCTGGATCCTCC
TCTTCTCATAGCAAAACCCAACAAAGTCCAGCTCATTGCCATGGATCTGCCCATGGTGAGTGGTGACCGG
ATCCACTGTCTTGACATCTTATTTGCTTTTACAAAGCGTGTTTTGGGTGAGAGTGGAGAGATGGATGCCC
TTCGAATACAGATGGAAGAGCGATTCATGGCATCAAACCCCTCCAAAGTCTCTTATGAGCCCATTACGAC
CACGTTGAAACGCAAACAAGAGGAGGTGTCTGCTATTATTATCCAGAGGGCTTACAGACGCTACCTCTTG
AAGCAAAAAGTTAAAAAGGTATCAAGTATATACAAGAAAGACAAAGGCAAAGAATGTGATGGAACACCCA
TCAAAGAAGATACTCTCATTGATAAACTGAATGAGAATTCAACTCCAGAGAAAACCGATATGACGCCTTC
CACCACGTCTCCACCCTCGTATGATAGTGTGACCAAACCAGAAAAAGAAAAATTTGAAAAAGACAAATCA
GAAAAGGAAGACAAAGGGAAAGATATCAGGGAAAGTAAAAAGTAAAAAGAAACCAAGAATTTTCCATTTT
GTGATCAATTGTTTACAGCCCGTGATGGTGATGTGTTTGTGTCAACAGGACTCCCACAGGAGGTCTATGC
CAAACTGACTGTTTTTACAAATGTATACTTAAGGTCAGTGCCTATAACAAGACAGAGACCTCTGGTCAGC
AAACTGGAACTCAGTAAACTGGAGAAATAGTATCGATGGGAGGTTTCTATTTTCACAACCAGCTGACACT
GCTGAAGAGCAGAGGCGTAATGGCTACTCAGACGATAGGAACCAATTTAAAGGGGGGAGGGAAGTTAAAT
TTTTATGTAAATTCAACATGTGACACTTGATAATAGTAATTGTCACCAGTGTTTATGTTTTAACTGCCAC
ACCTGCCATATTTTTACAAAACGTGTGCTGTGAATTTATCACTTTTCTTTTTAATTCACAGGTTGTTTAC
TATTATATGTGACTATTTTTGTAAATGGGTTTGTGTTTGGGGAGAGGGATTAAAGGGAGGGAATTCTACA
TTTCTCTATTGTATTGTATAACTGGATATATTTTAAATGGAGGCATGCTGCAATTCTCATTCACACATAA
AAAAATCACATCACAAAAGGGAAGAGTTTACTTCTTGTTTCAGGATGTTTTTAGATTTTTGAGGTGCTTA
AATAGCTATTCGTATTTTTAAGGTGTCTCATCCAGAAAAAATTTAATGTGCCTGTAAATGTTCCATAGAA
TCACAAGCATTAAAGAGTTGTTTTATTTTTACATAACCCATTAAATGTACATGTATATATGTATATATGT
ATATGTGCGTGTATATACATATATATGTATACACACATGCACACACAGAGATATACACATACCATTACAT
TGTCATTCACAGTCCCAGCAGCATGACTATCACATTTTTGATAAGTGTCCTTTGGCATAAAATAAAAATA
TCCTATCAGTCCTTTCTAAGAAGCCTGAATTGACCAAAAAACATCCCCACCACCACTTTATAAAGTTGAT
TCTGCTTTATCCTGCAGTATTGTTTAGCCATCTTCTGCTCTTGGTAAGGTTGACATAGTATATGTCAATT
TAAAAAATAAAAGTCTGCTTTGTAAATAGTAATTTTACCCAGTGGTGCATGTTTGAGCAAACAAAAATGA
TGATTTAAGCACACTACTTATTGCATCAAATATGTACCACAGTAAGTATAGTTTGCAAGCTTTCAACAGG
TAATATGATGTAATTGGTTCCATTATAGTTTGAAGCTGTCACTGCTGCATGTTTATCTTGCCTATGCTGC
TGTATCTTATTCCTTCCACTGTTCAGAAGTCTAATATGGGAAGCCATATATCAGTGGTAAAGTGAAGCAA
ATTGTTCTACCAAGACCTCATTCTTCATGTCATTAAGCAATAGGTTGCAGCAAACAAGGAAGAGCTTCTT
GCTTTTTATTCTTCCAACCTTAATTGAACACTCAATGATGAAAAGCCCGACTGTACAAACATGTTGCAAG
CTGCTTAAATCTGTTTAAAATATATGGTTAGAGTTTTCTAAGAAAATATAAATACTGTAAAAAGTTCATT
TTATTTTATTTTTCAGCCTTTTGTACGTAAAATGAGAAATTAAAAGTATCTTCAGGTGGATGTCACAGTC
ACTATTGTTAGTTTCTGTTCCTAGCACTTTTAAATTGAAGCACTTCACAAAATAAGAAGCAAGGACTAGG
ATGCAGTGTAGGTTTCTGCTTTTTTATTAGTACTGTAAACTTGCACACATTTCAATGTGAAACAAATCTC
AAACTGAGTTCAATGTTTATTTGCTTTCAATAGTAATGCCTTATCATTGAAAGAGGCTTAAAGAAAAAAA
AAATCAGCTGATACTCTTGGCATTGCTTGAATCCAATGTTTCCACCTAGTCTTTTTATTCAGTAATCATC
AGTCTTTTCCAATGTTTGTTTACACAGATAGATCTTATTGACCCATATGGCACTAGAACTGTATCAGATA
TAATATGGGATCCCAGCTTTTTTTCCTCTCCCACAAAACCAGGTAGTGAAGTTATATTACCAGTTACAGC
AAAATACTTTGTGTTTCACAAGCAACAATAAATGTAGATTCTTTATACTGAAGCTATTGACTTGTAGTGT
GTTGGTGAAATGCATGCAGGAAAATGCTGTTACCATAAAGAACGGTAAACCACATTACAATCAAGCCAAA
AGAATAAAGGTTTCGCTTTTGTTTTTGTATTTAATTGTTGTCTTTGTTTCTATCTTTGAAATGCCATTTA
AAGGTAGATTTCTATCATGTAAAAATAATCTATCTGAAAAACAAATGTAAAGAACACACATTAATTACTA
TAATTCATCTTTCAATTTTTTCATGGAATGGAAGTTAATTAAGAAGAGTGTATTGGATAACTACTTTAAT
ATTGGCCAAAAAGCTAGATATGGCATCAGGTAGACTAGTGGAAAGTTACAAAAATTAATAAAAAATTGAC
TAACA
SEQ ID NO: 5; SCN2A protein isoform 2 (NP_001035233.1):
MAQSVLVPPGPDSFRFFTRESLAAIEQRIAEEKAKRPKQERKDEDDENGPKPNSDLEAGKSLPFLYGDIP
PEMVSVPLEDLDPYYINKKTFIVLNKGKAISRFSATPALYILTPFNPIRKLAIKILVHSLFNMLIMCTIL
TNCVFMTMSNPPDWTKNVEYTFTGIYTFESLIKILARGFCLEDFTFLRDPWNWLDFTVITFAYVTEFVNL
GNVSALRTFRVLRALKTISVIPGLKTIVGALIQSVKKLSDVMILTVFCLSVFALIGLQLFMGNLRNKCLQ
WPPDNSSFEINITSFFNNSLDGNGTTFNRTVSIFNWDEYIEDKSHFYFLEGQNDALLCGNSSDAGQCPEG
YICVKAGRNPNYGYTSFDTFSWAFLSLFRLMTQDFWENLYQLTLRAAGKTYMIFFVLVIFLGSFYLINLI
LAVVAMAYEEQNQATLEEAEQKEAEFQQMLEQLKKQQEEAQAAAAAASAESRDFSGAGGIGVFSESSSVA
SKLSSKSEKELKNRRKKKKQKEQSGEEEKNDRVRKSESEDSIRRKGFRFSLEGSRLTYEKRFSSPHQSLL
SIRGSLFSPRRNSRASLFSFRGRAKDIGSENDFADDEHSTFEDNDSRRDSLFVPHRHGERRHSNVSQASR
ASRVLPILPMNGKMHSAVDCNGVVSLVGGPSTLTSAGQLLPEGTTTETEIRKRRSSSYHVSMDLLEDPTS
RQRAMSIASILTNTMEELEESRQKCPPCWYKFANMCLIWDCCKPWLKVKHLVNLVVMDPFVDLAITICIV
LNTLFMAMEHYPMTEQFSSVLSVGNLVFTGIFTAEMFLKIIAMDPYYYFQEGWNIFDGFIVSLSLMELGL
ANVEGLSVLRSFRLLRVFKLAKSWPTLNMLIKIIGNSVGALGNLTLVLAIIVFIFAVVGMQLFGKSYKEC
VCKISNDCELPRWHMHDFFHSFLIVFRVLCGEWIETMWDCMEVAGQTMCLTVFMMVMVIGNLVVLNLFLA
LLLSSFSSDNLAATDDDNEMNNLQIAVGRMQKGIDFVKRKIREFIQKAFVRKQKALDEIKPLEDLNNKKD
SCISNHTTIEIGKDLNYLKDGNGTTSGIGSSVEKYVVDESDYMSFINNPSLTVTVPIAVGESDFENLNTE
EFSSESDMEESKEKLNATSSSEGSTVDIGAPAEGEQPEVEPEESLEPEACFTEDCVRKFKCCQISIEEGK
GKLWWNLRKTCYKIVEHNWFETFIVFMILLSSGALAFEDIYIEQRKTIKTMLEYADKVFTYIFILEMLLK
WVAYGFQVYFTNAWCWLDFLIVDVSLVSLTANALGYSELGAIKSLRTLRALRPLRALSRFEGMRVVVNAL
LGAIPSIMNVLLVCLIFWLIFSIMGVNLFAGKFYHCINYTTGEMFDVSVVNNYSECKALIESNQTARWKN
VKVNFDNVGLGYLSLLQVATFKGWMDIMYAAVDSRNVELQPKYEDNLYMYLYFVIFIIFGSFFTLNLFIG
VIIDNFNQQKKKFGGQDIFMTEEQKKYYNAMKKLGSKKPQKPIPRPANKFQGMVFDFVTKQVFDISIMIL
ICLNMVTMMVETDDQSQEMTNILYWINLVFIVLFTGECVLKLISLRYYYFTIGWNIFDFVVVILSIVGMF
LAELIEKYFVSPTLFRVIRLARIGRILRLIKGAKGIRTLLFALMMSLPALFNIGLLLFLVMFIYAIFGMS
NFAYVKREVGIDDMENFETFGNSMICLFQITTSAGWDGLLAPILNSGPPDCDPDKDHPGSSVKGDCGNPS
VGIFFFVSYIIISFLVVVNMYIAVILENFSVATEESAEPLSEDDFEMFYEVWEKFDPDATQFIEFAKLSD
FADALDPPLLIAKPNKVQLIAMDLPMVSGDRIHCLDILFAFTKRVLGESGEMDALRIQMEERFMASNPSK
VSYEPITTTLKRKQEEVSAIIIQRAYRRYLLKQKVKKVSSIYKKDKGKECDGTPIKEDTLIDKLNENSTP
EKTDMTPSTTSPPSYDSVTKPEKEKFEKDKSEKEDKGKDIRESKK
SEQ ID NO: 6; SCN2A transcript variant 4 (NM_001371246.1)
AACAGACATTGGGTACCATCGAATGACTGTCAGAACAGAAAGCTAAGGCAAAGGAGGGAGGATGCTGTGG
TCATCCTTTCTTGTTTTTTTCTTCTTTAATGAGGATAGAGCACATGTGAGATTTTACTTTCTACTCCAGT
AAAAATTCTGAAGAATTGCATTGGAGACTGTTATATTCAACACATACGTGGATTCTGTGTTATGATTTAC
ATTTTTCTTTATTTCAGCACTTTCTTATGCAAGGAGCTAAACAGTGATTAAAGGAGCAGGATGAAAAGAT
GGCACAGTCAGTGCTGGTACCGCCAGGACCTGACAGCTTCCGCTTCTTTACCAGGGAATCCCTTGCTGCT
ATTGAACAACGCATTGCAGAAGAGAAAGCTAAGAGACCCAAACAGGAACGCAAGGATGAGGATGATGAAA
ATGGCCCAAAGCCAAACAGTGACTTGGAAGCAGGAAAATCTCTTCCATTTATTTATGGAGACATTCCTCC
AGAGATGGTGTCAGTGCCCCTGGAGGATCTGGACCCCTACTATATCAATAAGAAAACGTTTATAGTATTG
AATAAAGGGAAAGCAATCTCTCGATTCAGTGCCACCCCTGCCCTTTACATTTTAACTCCCTTCAACCCTA
TTAGAAAATTAGCTATTAAGATTTTGGTACATTCTTTATTCAATATGCTCATTATGTGCACGATTCTTAC
CAACTGTGTATTTATGACCATGAGTAACCCTCCAGACTGGACAAAGAATGTGGAGTATACCTTTACAGGA
ATTTATACTTTTGAATCACTTATTAAAATACTTGCAAGGGGCTTTTGTTTAGAAGATTTCACATTTTTAC
GGGATCCATGGAATTGGTTGGATTTCACAGTCATTACTTTTGCGTATGTAACAGAATTTGTAAACCTAGG
CAATGTTTCAGCTCTTCGAACTTTCAGAGTCTTGAGAGCTTTGAAAACTATTTCTGTAATTCCAGGCCTG
AAGACCATTGTGGGGGCCCTGATCCAGTCAGTGAAGAAGCTTTCTGATGTCATGATCTTGACTGTGTTCT
GTCTAAGCGTGTTTGCGCTAATAGGATTGCAGTTGTTCATGGGCAACCTACGAAATAAATGTTTGCAATG
GCCTCCAGATAATTCTTCCTTTGAAATAAATATCACTTCCTTCTTTAACAATTCATTGGATGGGAATGGT
ACTACTTTCAATAGGACAGTGAGCATATTTAACTGGGATGAATATATTGAGGATAAAAGTCACTTTTATT
TTTTAGAGGGGCAAAATGATGCTCTGCTTTGTGGCAACAGCTCAGATGCAGGCCAGTGTCCTGAAGGATA
CATCTGTGTGAAGGCTGGTAGAAACCCCAACTATGGCTACACGAGCTTTGACACCTTTAGTTGGGCCTTT
TTGTCCTTATTTCGTCTCATGACTCAAGACTTCTGGGAAAACCTTTATCAACTGACACTACGTGCTGCTG
GGAAAACGTACATGATATTTTTTGTGCTGGTCATTTTCTTGGGCTCATTCTATCTAATAAATTTGATCTT
GGCTGTGGTGGCCATGGCCTATGAGGAACAGAATCAGGCCACATTGGAAGAGGCTGAACAGAAGGAAGCT
GAATTTCAGCAGATGCTCGAACAGTTGAAAAAGCAACAAGAAGAAGCTCAGGCGGCAGCTGCAGCCGCAT
CTGCTGAATCAAGAGACTTCAGTGGTGCTGGTGGGATAGGAGTTTTTTCAGAGAGTTCTTCAGTAGCATC
TAAGTTGAGCTCCAAAAGTGAAAAAGAGCTGAAAAACAGAAGAAAGAAAAAGAAACAGAAAGAACAGTCT
GGAGAAGAAGAGAAAAATGACAGAGTCCGAAAATCGGAATCTGAAGACAGCATAAGAAGAAAAGGTTTCC
GTTTTTCCTTGGAAGGAAGTAGGCTGACATATGAAAAGAGATTTTCTTCTCCACACCAGTCCTTACTGAG
CATCCGTGGCTCCCTTTTCTCTCCAAGACGCAACAGTAGGGCGAGCCTTTTCAGCTTCAGAGGTCGAGCA
AAGGACATTGGCTCTGAGAATGACTTTGCTGATGATGAGCACAGCACCTTTGAGGACAATGACAGCCGAA
GAGACTCTCTGTTCGTGCCGCACAGACATGGAGAACGGCGCCACAGCAATGTCAGCCAGGCCAGCCGTGC
CTCCAGGGTGCTCCCCATCCTGCCCATGAATGGGAAGATGCATAGCGCTGTGGACTGCAATGGTGTGGTC
TCCCTGGTCGGGGGCCCTTCTACCCTCACATCTGCTGGGCAGCTCCTACCAGAGGGCACAACTACTGAAA
CAGAAATAAGAAAGAGACGGTCCAGTTCTTATCATGTTTCCATGGATTTATTGGAAGATCCTACATCAAG
GCAAAGAGCAATGAGTATAGCCAGTATTTTGACCAACACCATGGAAGAACTTGAAGAATCCAGACAGAAA
TGCCCACCATGCTGGTATAAATTTGCTAATATGTGTTTGATTTGGGACTGTTGTAAACCATGGTTAAAGG
TGAAACACCTTGTCAACCTGGTTGTAATGGACCCATTTGTTGACCTGGCCATCACCATCTGCATTGTCTT
AAATACACTCTTCATGGCTATGGAGCACTATCCCATGACGGAGCAGTTCAGCAGTGTACTGTCTGTTGGA
AACCTGGTCTTCACAGGGATCTTCACAGCAGAAATGTTTCTCAAGATAATTGCCATGGATCCATATTATT
ACTTTCAAGAAGGCTGGAATATTTTTGATGGTTTTATTGTGAGCCTTAGTTTAATGGAACTTGGTTTGGC
AAATGTGGAAGGATTGTCAGTTCTCCGATCATTCCGGCTGCTCCGAGTTTTCAAGTTGGCAAAATCTTGG
CCAACTCTAAATATGCTAATTAAGATCATTGGCAATTCTGTGGGGGCTCTAGGAAACCTCACCTTGGTAT
TGGCCATCATCGTCTTCATTTTTGCTGTGGTCGGCATGCAGCTCTTTGGTAAGAGCTACAAAGAATGTGT
CTGCAAGATTTCCAATGATTGTGAACTCCCACGCTGGCACATGCATGACTTTTTCCACTCCTTCCTGATC
GTGTTCCGCGTGCTGTGTGGAGAGTGGATAGAGACCATGTGGGACTGTATGGAGGTCGCTGGCCAAACCA
TGTGCCTTACTGTCTTCATGATGGTCATGGTGATTGGAAATCTAGTGGTTCTGAACCTCTTCTTGGCCTT
GCTTTTGAGTTCCTTCAGTTCTGACAATCTTGCTGCCACTGATGATGATAACGAAATGAATAATCTCCAG
ATTGCTGTGGGAAGGATGCAGAAAGGAATCGATTTTGTTAAAAGAAAAATACGTGAATTTATTCAGAAAG
CCTTTGTTAGGAAGCAGAAAGCTTTAGATGAAATTAAACCGCTTGAAGATCTAAATAATAAAAAAGACAG
CTGTATTTCCAACCATACCACCATAGAAATAGGCAAAGACCTCAATTATCTCAAAGACGGAAATGGAACT
ACTAGTGGCATAGGCAGCAGTGTAGAAAAATATGTCGTGGATGAAAGTGATTACATGTCATTTATAAACA
ACCCTAGCCTCACTGTGACAGTACCAATTGCTGTTGGAGAATCTGACTTTGAAAATTTAAATACTGAAGA
ATTCAGCAGCGAGTCAGATATGGAGGAAAGCAAAGAGAAGCTAAATGCAACTAGTTCATCTGAAGGCAGC
ACGGTTGATATTGGAGCTCCCGCCGAGGGAGAACAGCCTGAGGTTGAACCTGAGGAATCCCTTGAACCTG
AAGCCTGTTTTACAGAAGACTGTGTACGGAAGTTCAAGTGTTGTCAGATAAGCATAGAAGAAGGCAAAGG
GAAACTCTGGTGGAATTTGAGGAAAACATGCTATAAGATAGTGGAGCACAATTGGTTCGAAACCTTCATT
GTCTTCATGATTCTGCTGAGCAGTGGGGCTCTGGCCTTTGAAGATATATACATTGAGCAGCGAAAAACCA
TTAAGACCATGTTAGAATATGCTGACAAGGTTTTCACTTACATATTCATTCTGGAAATGCTGCTAAAGTG
GGTTGCATATGGTTTTCAAGTGTATTTTACCAATGCCTGGTGCTGGCTAGACTTCCTGATTGTTGATGTC
TCACTGGTTAGCTTAACTGCAAATGCCTTGGGTTACTCAGAACTTGGTGCCATCAAATCCCTCAGAACAC
TAAGAGCTCTGAGGCCACTGAGAGCTTTGTCCCGGTTTGAAGGAATGAGGGTTGTTGTAAATGCTCTTTT
AGGAGCCATTCCATCTATCATGAATGTACTTCTGGTTTGTCTGATCTTTTGGCTAATATTCAGTATCATG
GGAGTGAATCTCTTTGCTGGCAAGTTTTACCATTGTATTAATTACACCACTGGAGAGATGTTTGATGTAA
GCGTGGTCAACAACTACAGTGAGTGCAAAGCTCTCATTGAGAGCAATCAAACTGCCAGGTGGAAAAATGT
GAAAGTAAACTTTGATAACGTAGGACTTGGATATCTGTCTCTACTTCAAGTAGCCACGTTTAAGGGATGG
ATGGATATTATGTATGCAGCTGTTGATTCACGAAATGTAGAATTACAACCCAAGTATGAAGACAACCTGT
ACATGTATCTTTATTTTGTCATCTTTATTATTTTTGGTTCATTCTTTACCTTGAATCTTTTCATTGGTGT
CATCATAGATAACTTCAACCAACAGAAAAAGAAGTTTGGAGGTCAAGACATTTTTATGACAGAAGAACAG
AAGAAATACTACAATGCAATGAAAAAACTGGGTTCAAAGAAACCACAAAAACCCATACCTCGACCTGCTA
ACAAATTCCAAGGAATGGTCTTTGATTTTGTAACCAAACAAGTCTTTGATATCAGCATCATGATCCTCAT
CTGCCTTAACATGGTCACCATGATGGTGGAAACCGATGACCAGAGTCAAGAAATGACAAACATTCTGTAC
TGGATTAATCTGGTGTTTATTGTTCTGTTCACTGGAGAATGTGTGCTGAAACTGATCTCTCTTCGTTACT
ACTATTTCACTATTGGATGGAATATTTTTGATTTTGTGGTGGTCATTCTCTCCATTGTAGGAATGTTTCT
GGCTGAACTGATAGAAAAGTATTTTGTGTCCCCTACCCTGTTCCGAGTGATCCGTCTTGCCAGGATTGGC
CGAATCCTACGTCTGATCAAAGGAGCAAAGGGGATCCGCACGCTGCTCTTTGCTTTGATGATGTCCCTTC
CTGCGTTGTTTAACATCGGCCTCCTTCTTTTCCTGGTCATGTTCATCTACGCCATCTTTGGGATGTCCAA
TTTTGCCTATGTTAAGAGGGAAGTTGGGATCGATGACATGTTCAACTTTGAGACCTTTGGCAACAGCATG
ATCTGCCTGTTCCAAATTACAACCTCTGCTGGCTGGGATGGATTGCTAGCACCTATTCTTAATAGTGGAC
CTCCAGACTGTGACCCTGACAAAGATCACCCTGGAAGCTCAGTTAAAGGAGACTGTGGGAACCCATCTGT
TGGGATTTTCTTTTTTGTCAGTTACATCATCATATCCTTCCTGGTTGTGGTGAACATGTACATCGCGGTC
ATCCTGGAGAACTTCAGTGTTGCTACTGAAGAAAGTGCAGAGCCTCTGAGTGAGGATGACTTTGAGATGT
TCTATGAGGTTTGGGAGAAGTTTGATCCCGATGCGACCCAGTTTATAGAGTTTGCCAAACTTTCTGATTT
TGCAGATGCCCTGGATCCTCCTCTTCTCATAGCAAAACCCAACAAAGTCCAGCTCATTGCCATGGATCTG
CCCATGGTGAGTGGTGACCGGATCCACTGTCTTGACATCTTATTTGCTTTTACAAAGCGTGTTTTGGGTG
AGAGTGGAGAGATGGATGCCCTTCGAATACAGATGGAAGAGCGATTCATGGCATCAAACCCCTCCAAAGT
CTCTTATGAGCCCATTACGACCACGTTGAAACGCAAACAAGAGGAGGTGTCTGCTATTATTATCCAGAGG
GCTTACAGACGCTACCTCTTGAAGCAAAAAGTTAAAAAGGTATCAAGTATATACAAGAAAGACAAAGGCA
AAGAATGTGATGGAACACCCATCAAAGAAGATACTCTCATTGATAAACTGAATGAGAATTCAACTCCAGA
GAAAACCGATATGACGCCTTCCACCACGTCTCCACCCTCGTATGATAGTGTGACCAAACCAGAAAAAGAA
AAATTTGAAAAAGACAAATCAGAAAAGGAAGACAAAGGGAAAGATATCAGGGAAAGTAAAAAGTAAAAAG
AAACCAAGAATTTTCCATTTTGTGATCAATTGTTTACAGCCCGTGATGGTGATGTGTTTGTGTCAACAGG
ACTCCCACAGGAGGTCTATGCCAAACTGACTGTTTTTACAAATGTATACTTAAGGTCAGTGCCTATAACA
AGACAGAGACCTCTGGTCAGCAAACTGGAACTCAGTAAACTGGAGAAATAGTATCGATGGGAGGTTTCTA
TTTTCACAACCAGCTGACACTGCTGAAGAGCAGAGGCGTAATGGCTACTCAGACGATAGGAACCAATTTA
AAGGGGGGAGGGAAGTTAAATTTTTATGTAAATTCAACATGTGACACTTGATAATAGTAATTGTCACCAG
TGTTTATGTTTTAACTGCCACACCTGCCATATTTTTACAAAACGTGTGCTGTGAATTTATCACTTTTCTT
TTTAATTCACAGGTTGTTTACTATTATATGTGACTATTTTTGTAAATGGGTTTGTGTTTGGGGAGAGGGA
TTAAAGGGAGGGAATTCTACATTTCTCTATTGTATTGTATAACTGGATATATTTTAAATGGAGGCATGCT
GCAATTCTCATTCACACATAAAAAAATCACATCACAAAAGGGAAGAGTTTACTTCTTGTTTCAGGATGTT
TTTAGATTTTTGAGGTGCTTAAATAGCTATTCGTATTTTTAAGGTGTCTCATCCAGAAAAAATTTAATGT
GCCTGTAAATGTTCCATAGAATCACAAGCATTAAAGAGTTGTTTTATTTTTACATAACCCATTAAATGTA
CATGTATATATGTATATATGTATATGTGCGTGTATATACATATATATGTATACACACATGCACACACAGA
GATATACACATACCATTACATTGTCATTCACAGTCCCAGCAGCATGACTATCACATTTTTGATAAGTGTC
CTTTGGCATAAAATAAAAATATCCTATCAGTCCTTTCTAAGAAGCCTGAATTGACCAAAAAACATCCCCA
CCACCACTTTATAAAGTTGATTCTGCTTTATCCTGCAGTATTGTTTAGCCATCTTCTGCTCTTGGTAAGG
TTGACATAGTATATGTCAATTTAAAAAATAAAAGTCTGCTTTGTAAATAGTAATTTTACCCAGTGGTGCA
TGTTTGAGCAAACAAAAATGATGATTTAAGCACACTACTTATTGCATCAAATATGTACCACAGTAAGTAT
AGTTTGCAAGCTTTCAACAGGTAATATGATGTAATTGGTTCCATTATAGTTTGAAGCTGTCACTGCTGCA
TGTTTATCTTGCCTATGCTGCTGTATCTTATTCCTTCCACTGTTCAGAAGTCTAATATGGGAAGCCATAT
ATCAGTGGTAAAGTGAAGCAAATTGTTCTACCAAGACCTCATTCTTCATGTCATTAAGCAATAGGTTGCA
GCAAACAAGGAAGAGCTTCTTGCTTTTTATTCTTCCAACCTTAATTGAACACTCAATGATGAAAAGCCCG
ACTGTACAAACATGTTGCAAGCTGCTTAAATCTGTTTAAAATATATGGTTAGAGTTTTCTAAGAAAATAT
AAATACTGTAAAAAGTTCATTTTATTTTATTTTTCAGCCTTTTGTACGTAAAATGAGAAATTAAAAGTAT
CTTCAGGTGGATGTCACAGTCACTATTGTTAGTTTCTGTTCCTAGCACTTTTAAATTGAAGCACTTCACA
AAATAAGAAGCAAGGACTAGGATGCAGTGTAGGTTTCTGCTTTTTTATTAGTACTGTAAACTTGCACACA
TTTCAATGTGAAACAAATCTCAAACTGAGTTCAATGTTTATTTGCTTTCAATAGTAATGCCTTATCATTG
AAAGAGGCTTAAAGAAAAAAAAAATCAGCTGATACTCTTGGCATTGCTTGAATCCAATGTTTCCACCTAG
TCTTTTTATTCAGTAATCATCAGTCTTTTCCAATGTTTGTTTACACAGATAGATCTTATTGACCCATATG
GCACTAGAACTGTATCAGATATAATATGGGATCCCAGCTTTTTTTCCTCTCCCACAAAACCAGGTAGTGA
AGTTATATTACCAGTTACAGCAAAATACTTTGTGTTTCACAAGCAACAATAAATGTAGATTCTTTATACT
GAAGCTATTGACTTGTAGTGTGTTGGTGAAATGCATGCAGGAAAATGCTGTTACCATAAAGAACGGTAAA
CCACATTACAATCAAGCCAAAAGAATAAAGGTTTCGCTTTTGTTTTTGTATTTAATTGTTGTCTTTGTTT
CTATCTTTGAAATGCCATTTAAAGGTAGATTTCTATCATGTAAAAATAATCTATCTGAAAAACAAATGTA
AAGAACACACATTAATTACTATAATTCATCTTTCAATTTTTTCATGGAATGGAAGTTAATTAAGAAGAGT
GTATTGGATAACTACTTTAATATTGGCCAAAAAGCTAGATATGGCATCAGGTAGACTAGTGGAAAGTTAC
AAAAATTAATAAAAAATTGACTAACA
SEQ ID NO: 7; SCN2A protein isoform 2 (NP_001358175.1):
MAQSVLVPPGPDSFRFFTRESLAAIEQRIAEEKAKRPKQERKDEDDENGPKPNSDLEAGKSLPFLYGDIP
PEMVSVPLEDLDPYYINKKTFIVLNKGKAISRFSATPALYILTPFNPIRKLAIKILVHSLFNMLIMCTIL
TNCVFMTMSNPPDWTKNVEYTFTGIYTFESLIKILARGFCLEDFTFLRDPWNWLDFTVITFAYVTEFVNL
GNVSALRTFRVLRALKTISVIPGLKTIVGALIQSVKKLSDVMILTVFCLSVFALIGLQLFMGNLRNKCLQ
WPPDNSSFEINITSFFNNSLDGNGTTFNRTVSIFNWDEYIEDKSHFYFLEGQNDALLCGNSSDAGQCPEG
YICVKAGRNPNYGYTSFDTFSWAFLSLFRLMTQDFWENLYQLTLRAAGKTYMIFFVLVIFLGSFYLINLI
LAVVAMAYEEQNQATLEEAEQKEAEFQQMLEQLKKQQEEAQAAAAAASAESRDFSGAGGIGVFSESSSVA
SKLSSKSEKELKNRRKKKKQKEQSGEEEKNDRVRKSESEDSIRRKGFRFSLEGSRLTYEKRFSSPHQSLL
SIRGSLFSPRRNSRASLFSFRGRAKDIGSENDFADDEHSTFEDNDSRRDSLFVPHRHGERRHSNVSQASR
ASRVLPILPMNGKMHSAVDCNGVVSLVGGPSTLTSAGQLLPEGTTTETEIRKRRSSSYHVSMDLLEDPTS
RQRAMSIASILTNTMEELEESRQKCPPCWYKFANMCLIWDCCKPWLKVKHLVNLVVMDPFVDLAITICIV
LNTLFMAMEHYPMTEQFSSVLSVGNLVFTGIFTAEMFLKIIAMDPYYYFQEGWNIFDGFIVSLSLMELGL
ANVEGLSVLRSFRLLRVFKLAKSWPTLNMLIKIIGNSVGALGNLTLVLAIIVFIFAVVGMQLFGKSYKEC
VCKISNDCELPRWHMHDFFHSFLIVFRVLCGEWIETMWDCMEVAGQTMCLTVFMMVMVIGNLVVLNLFLA
LLLSSFSSDNLAATDDDNEMNNLQIAVGRMQKGIDFVKRKIREFIQKAFVRKQKALDEIKPLEDLNNKKD
SCISNHTTIEIGKDLNYLKDGNGTTSGIGSSVEKYVVDESDYMSFINNPSLTVTVPIAVGESDFENLNTE
EFSSESDMEESKEKLNATSSSEGSTVDIGAPAEGEQPEVEPEESLEPEACFTEDCVRKFKCCQISIEEGK
GKLWWNLRKTCYKIVEHNWFETFIVFMILLSSGALAFEDIYIEQRKTIKTMLEYADKVFTYIFILEMLLK
WVAYGFQVYFTNAWCWLDFLIVDVSLVSLTANALGYSELGAIKSLRTLRALRPLRALSRFEGMRVVVNAL
LGAIPSIMNVLLVCLIFWLIFSIMGVNLFAGKFYHCINYTTGEMFDVSVVNNYSECKALIESNQTARWKN
VKVNFDNVGLGYLSLLQVATFKGWMDIMYAAVDSRNVELQPKYEDNLYMYLYFVIFIIFGSFFTLNLFIG
VIIDNFNQQKKKFGGQDIFMTEEQKKYYNAMKKLGSKKPQKPIPRPANKFQGMVFDFVTKQVFDISIMIL
ICLNMVTMMVETDDQSQEMTNILYWINLVFIVLFTGECVLKLISLRYYYFTIGWNIFDFVVVILSIVGMF
LAELIEKYFVSPTLFRVIRLARIGRILRLIKGAKGIRTLLFALMMSLPALFNIGLLLFLVMFIYAIFGMS
NFAYVKREVGIDDMFNFETFGNSMICLFQITTSAGWDGLLAPILNSGPPDCDPDKDHPGSSVKGDCGNPS
VGIFFFVSYIIISFLVVVNMYIAVILENFSVATEESAEPLSEDDFEMFYEVWEKFDPDATQFIEFAKLSD
FADALDPPLLIAKPNKVQLIAMDLPMVSGDRIHCLDILFAFTKRVLGESGEMDALRIQMEERFMASNPSK
VSYEPITTTLKRKQEEVSAIIIQRAYRRYLLKQKVKKVSSIYKKDKGKECDGTPIKEDTLIDKLNENSTP
EKTDMTPSTTSPPSYDSVTKPEKEKFEKDKSEKEDKGKDIRESKK
SEQ ID NO: 8; SCN2A transcript variant 5 (NM_001371247.1):
ATAGCAGTAACACAATTCACCTCTAGTGTGAACATATCAGGATGGCATAGACCAGCACTTTCTTATGCAA
GGAGCTAAACAGTGATTAAAGGAGCAGGATGAAAAGATGGCACAGTCAGTGCTGGTACCGCCAGGACCTG
ACAGCTTCCGCTTCTTTACCAGGGAATCCCTTGCTGCTATTGAACAACGCATTGCAGAAGAGAAAGCTAA
GAGACCCAAACAGGAACGCAAGGATGAGGATGATGAAAATGGCCCAAAGCCAAACAGTGACTTGGAAGCA
GGAAAATCTCTTCCATTTATTTATGGAGACATTCCTCCAGAGATGGTGTCAGTGCCCCTGGAGGATCTGG
ACCCCTACTATATCAATAAGAAAACGTTTATAGTATTGAATAAAGGGAAAGCAATCTCTCGATTCAGTGC
CACCCCTGCCCTTTACATTTTAACTCCCTTCAACCCTATTAGAAAATTAGCTATTAAGATTTTGGTACAT
TCTTTATTCAATATGCTCATTATGTGCACGATTCTTACCAACTGTGTATTTATGACCATGAGTAACCCTC
CAGACTGGACAAAGAATGTGGAGTATACCTTTACAGGAATTTATACTTTTGAATCACTTATTAAAATACT
TGCAAGGGGCTTTTGTTTAGAAGATTTCACATTTTTACGGGATCCATGGAATTGGTTGGATTTCACAGTC
ATTACTTTTGCATATGTGACAGAGTTTGTGGACCTGGGCAATGTCTCAGCGTTGAGAACATTCAGAGTTC
TCCGAGCATTGAAAACAATTTCAGTCATTCCAGGCCTGAAGACCATTGTGGGGGCCCTGATCCAGTCAGT
GAAGAAGCTTTCTGATGTCATGATCTTGACTGTGTTCTGTCTAAGCGTGTTTGCGCTAATAGGATTGCAG
TTGTTCATGGGCAACCTACGAAATAAATGTTTGCAATGGCCTCCAGATAATTCTTCCTTTGAAATAAATA
TCACTTCCTTCTTTAACAATTCATTGGATGGGAATGGTACTACTTTCAATAGGACAGTGAGCATATTTAA
CTGGGATGAATATATTGAGGATAAAAGTCACTTTTATTTTTTAGAGGGGCAAAATGATGCTCTGCTTTGT
GGCAACAGCTCAGATGCAGGCCAGTGTCCTGAAGGATACATCTGTGTGAAGGCTGGTAGAAACCCCAACT
ATGGCTACACGAGCTTTGACACCTTTAGTTGGGCCTTTTTGTCCTTATTTCGTCTCATGACTCAAGACTT
CTGGGAAAACCTTTATCAACTGACACTACGTGCTGCTGGGAAAACGTACATGATATTTTTTGTGCTGGTC
ATTTTCTTGGGCTCATTCTATCTAATAAATTTGATCTTGGCTGTGGTGGCCATGGCCTATGAGGAACAGA
ATCAGGCCACATTGGAAGAGGCTGAACAGAAGGAAGCTGAATTTCAGCAGATGCTCGAACAGTTGAAAAA
GCAACAAGAAGAAGCTCAGGCGGCAGCTGCAGCCGCATCTGCTGAATCAAGAGACTTCAGTGGTGCTGGT
GGGATAGGAGTTTTTTCAGAGAGTTCTTCAGTAGCATCTAAGTTGAGCTCCAAAAGTGAAAAAGAGCTGA
AAAACAGAAGAAAGAAAAAGAAACAGAAAGAACAGTCTGGAGAAGAAGAGAAAAATGACAGAGTCCGAAA
ATCGGAATCTGAAGACAGCATAAGAAGAAAAGGTTTCCGTTTTTCCTTGGAAGGAAGTAGGCTGACATAT
GAAAAGAGATTTTCTTCTCCACACCAGTCCTTACTGAGCATCCGTGGCTCCCTTTTCTCTCCAAGACGCA
ACAGTAGGGCGAGCCTTTTCAGCTTCAGAGGTCGAGCAAAGGACATTGGCTCTGAGAATGACTTTGCTGA
TGATGAGCACAGCACCTTTGAGGACAATGACAGCCGAAGAGACTCTCTGTTCGTGCCGCACAGACATGGA
GAACGGCGCCACAGCAATGTCAGCCAGGCCAGCCGTGCCTCCAGGGTGCTCCCCATCCTGCCCATGAATG
GGAAGATGCATAGCGCTGTGGACTGCAATGGTGTGGTCTCCCTGGTCGGGGGCCCTTCTACCCTCACATC
TGCTGGGCAGCTCCTACCAGAGGGCACAACTACTGAAACAGAAATAAGAAAGAGACGGTCCAGTTCTTAT
CATGTTTCCATGGATTTATTGGAAGATCCTACATCAAGGCAAAGAGCAATGAGTATAGCCAGTATTTTGA
CCAACACCATGGAAGAACTTGAAGAATCCAGACAGAAATGCCCACCATGCTGGTATAAATTTGCTAATAT
GTGTTTGATTTGGGACTGTTGTAAACCATGGTTAAAGGTGAAACACCTTGTCAACCTGGTTGTAATGGAC
CCATTTGTTGACCTGGCCATCACCATCTGCATTGTCTTAAATACACTCTTCATGGCTATGGAGCACTATC
CCATGACGGAGCAGTTCAGCAGTGTACTGTCTGTTGGAAACCTGGTCTTCACAGGGATCTTCACAGCAGA
AATGTTTCTCAAGATAATTGCCATGGATCCATATTATTACTTTCAAGAAGGCTGGAATATTTTTGATGGT
TTTATTGTGAGCCTTAGTTTAATGGAACTTGGTTTGGCAAATGTGGAAGGATTGTCAGTTCTCCGATCAT
TCCGGCTGCTCCGAGTTTTCAAGTTGGCAAAATCTTGGCCAACTCTAAATATGCTAATTAAGATCATTGG
CAATTCTGTGGGGGCTCTAGGAAACCTCACCTTGGTATTGGCCATCATCGTCTTCATTTTTGCTGTGGTC
GGCATGCAGCTCTTTGGTAAGAGCTACAAAGAATGTGTCTGCAAGATTTCCAATGATTGTGAACTCCCAC
GCTGGCACATGCATGACTTTTTCCACTCCTTCCTGATCGTGTTCCGCGTGCTGTGTGGAGAGTGGATAGA
GACCATGTGGGACTGTATGGAGGTCGCTGGCCAAACCATGTGCCTTACTGTCTTCATGATGGTCATGGTG
ATTGGAAATCTAGTGGTTCTGAACCTCTTCTTGGCCTTGCTTTTGAGTTCCTTCAGTTCTGACAATCTTG
CTGCCACTGATGATGATAACGAAATGAATAATCTCCAGATTGCTGTGGGAAGGATGCAGAAAGGAATCGA
TTTTGTTAAAAGAAAAATACGTGAATTTATTCAGAAAGCCTTTGTTAGGAAGCAGAAAGCTTTAGATGAA
ATTAAACCGCTTGAAGATCTAAATAATAAAAAAGACAGCTGTATTTCCAACCATACCACCATAGAAATAG
GCAAAGACCTCAATTATCTCAAAGACGGAAATGGAACTACTAGTGGCATAGGCAGCAGTGTAGAAAAATA
TGTCGTGGATGAAAGTGATTACATGTCATTTATAAACAACCCTAGCCTCACTGTGACAGTACCAATTGCT
GTTGGAGAATCTGACTTTGAAAATTTAAATACTGAAGAATTCAGCAGCGAGTCAGATATGGAGGAAAGCA
AAGAGAAGCTAAATGCAACTAGTTCATCTGAAGGCAGCACGGTTGATATTGGAGCTCCCGCCGAGGGAGA
ACAGCCTGAGGTTGAACCTGAGGAATCCCTTGAACCTGAAGCCTGTTTTACAGAAGACTGTGTACGGAAG
TTCAAGTGTTGTCAGATAAGCATAGAAGAAGGCAAAGGGAAACTCTGGTGGAATTTGAGGAAAACATGCT
ATAAGATAGTGGAGCACAATTGGTTCGAAACCTTCATTGTCTTCATGATTCTGCTGAGCAGTGGGGCTCT
GGCCTTTGAAGATATATACATTGAGCAGCGAAAAACCATTAAGACCATGTTAGAATATGCTGACAAGGTT
TTCACTTACATATTCATTCTGGAAATGCTGCTAAAGTGGGTTGCATATGGTTTTCAAGTGTATTTTACCA
ATGCCTGGTGCTGGCTAGACTTCCTGATTGTTGATGTCTCACTGGTTAGCTTAACTGCAAATGCCTTGGG
TTACTCAGAACTTGGTGCCATCAAATCCCTCAGAACACTAAGAGCTCTGAGGCCACTGAGAGCTTTGTCC
CGGTTTGAAGGAATGAGGGTTGTTGTAAATGCTCTTTTAGGAGCCATTCCATCTATCATGAATGTACTTC
TGGTTTGTCTGATCTTTTGGCTAATATTCAGTATCATGGGAGTGAATCTCTTTGCTGGCAAGTTTTACCA
TTGTATTAATTACACCACTGGAGAGATGTTTGATGTAAGCGTGGTCAACAACTACAGTGAGTGCAAAGCT
CTCATTGAGAGCAATCAAACTGCCAGGTGGAAAAATGTGAAAGTAAACTTTGATAACGTAGGACTTGGAT
ATCTGTCTCTACTTCAAGTAGCCACGTTTAAGGGATGGATGGATATTATGTATGCAGCTGTTGATTCACG
AAATGTAGAATTACAACCCAAGTATGAAGACAACCTGTACATGTATCTTTATTTTGTCATCTTTATTATT
TTTGGTTCATTCTTTACCTTGAATCTTTTCATTGGTGTCATCATAGATAACTTCAACCAACAGAAAAAGA
AGTTTGGAGGTCAAGACATTTTTATGACAGAAGAACAGAAGAAATACTACAATGCAATGAAAAAACTGGG
TTCAAAGAAACCACAAAAACCCATACCTCGACCTGCTAACAAATTCCAAGGAATGGTCTTTGATTTTGTA
ACCAAACAAGTCTTTGATATCAGCATCATGATCCTCATCTGCCTTAACATGGTCACCATGATGGTGGAAA
CCGATGACCAGAGTCAAGAAATGACAAACATTCTGTACTGGATTAATCTGGTGTTTATTGTTCTGTTCAC
TGGAGAATGTGTGCTGAAACTGATCTCTCTTCGTTACTACTATTTCACTATTGGATGGAATATTTTTGAT
TTTGTGGTGGTCATTCTCTCCATTGTAGGAATGTTTCTGGCTGAACTGATAGAAAAGTATTTTGTGTCCC
CTACCCTGTTCCGAGTGATCCGTCTTGCCAGGATTGGCCGAATCCTACGTCTGATCAAAGGAGCAAAGGG
GATCCGCACGCTGCTCTTTGCTTTGATGATGTCCCTTCCTGCGTTGTTTAACATCGGCCTCCTTCTTTTC
CTGGTCATGTTCATCTACGCCATCTTTGGGATGTCCAATTTTGCCTATGTTAAGAGGGAAGTTGGGATCG
ATGACATGTTCAACTTTGAGACCTTTGGCAACAGCATGATCTGCCTGTTCCAAATTACAACCTCTGCTGG
CTGGGATGGATTGCTAGCACCTATTCTTAATAGTGGACCTCCAGACTGTGACCCTGACAAAGATCACCCT
GGAAGCTCAGTTAAAGGAGACTGTGGGAACCCATCTGTTGGGATTTTCTTTTTTGTCAGTTACATCATCA
TATCCTTCCTGGTTGTGGTGAACATGTACATCGCGGTCATCCTGGAGAACTTCAGTGTTGCTACTGAAGA
AAGTGCAGAGCCTCTGAGTGAGGATGACTTTGAGATGTTCTATGAGGTTTGGGAGAAGTTTGATCCCGAT
GCGACCCAGTTTATAGAGTTTGCCAAACTTTCTGATTTTGCAGATGCCCTGGATCCTCCTCTTCTCATAG
CAAAACCCAACAAAGTCCAGCTCATTGCCATGGATCTGCCCATGGTGAGTGGTGACCGGATCCACTGTCT
TGACATCTTATTTGCTTTTACAAAGCGTGTTTTGGGTGAGAGTGGAGAGATGGATGCCCTTCGAATACAG
ATGGAAGAGCGATTCATGGCATCAAACCCCTCCAAAGTCTCTTATGAGCCCATTACGACCACGTTGAAAC
GCAAACAAGAGGAGGTGTCTGCTATTATTATCCAGAGGGCTTACAGACGCTACCTCTTGAAGCAAAAAGT
TAAAAAGGTATCAAGTATATACAAGAAAGACAAAGGCAAAGAATGTGATGGAACACCCATCAAAGAAGAT
ACTCTCATTGATAAACTGAATGAGAATTCAACTCCAGAGAAAACCGATATGACGCCTTCCACCACGTCTC
CACCCTCGTATGATAGTGTGACCAAACCAGAAAAAGAAAAATTTGAAAAAGACAAATCAGAAAAGGAAGA
CAAAGGGAAAGATATCAGGGAAAGTAAAAAGTAAAAAGAAACCAAGAATTTTCCATTTTGTGATCAATTG
TTTACAGCCCGTGATGGTGATGTGTTTGTGTCAACAGGACTCCCACAGGAGGTCTATGCCAAACTGACTG
TTTTTACAAATGTATACTTAAGGTCAGTGCCTATAACAAGACAGAGACCTCTGGTCAGCAAACTGGAACT
CAGTAAACTGGAGAAATAGTATCGATGGGAGGTTTCTATTTTCACAACCAGCTGACACTGCTGAAGAGCA
GAGGCGTAATGGCTACTCAGACGATAGGAACCAATTTAAAGGGGGGAGGGAAGTTAAATTTTTATGTAAA
TTCAACATGTGACACTTGATAATAGTAATTGTCACCAGTGTTTATGTTTTAACTGCCACACCTGCCATAT
TTTTACAAAACGTGTGCTGTGAATTTATCACTTTTCTTTTTAATTCACAGGTTGTTTACTATTATATGTG
ACTATTTTTGTAAATGGGTTTGTGTTTGGGGAGAGGGATTAAAGGGAGGGAATTCTACATTTCTCTATTG
TATTGTATAACTGGATATATTTTAAATGGAGGCATGCTGCAATTCTCATTCACACATAAAAAAATCACAT
CACAAAAGGGAAGAGTTTACTTCTTGTTTCAGGATGTTTTTAGATTTTTGAGGTGCTTAAATAGCTATTC
GTATTTTTAAGGTGTCTCATCCAGAAAAAATTTAATGTGCCTGTAAATGTTCCATAGAATCACAAGCATT
AAAGAGTTGTTTTATTTTTACATAACCCATTAAATGTACATGTATATATGTATATATGTATATGTGCGTG
TATATACATATATATGTATACACACATGCACACACAGAGATATACACATACCATTACATTGTCATTCACA
GTCCCAGCAGCATGACTATCACATTTTTGATAAGTGTCCTTTGGCATAAAATAAAAATATCCTATCAGTC
CTTTCTAAGAAGCCTGAATTGACCAAAAAACATCCCCACCACCACTTTATAAAGTTGATTCTGCTTTATC
CTGCAGTATTGTTTAGCCATCTTCTGCTCTTGGTAAGGTTGACATAGTATATGTCAATTTAAAAAATAAA
AGTCTGCTTTGTAAATAGTAATTTTACCCAGTGGTGCATGTTTGAGCAAACAAAAATGATGATTTAAGCA
CACTACTTATTGCATCAAATATGTACCACAGTAAGTATAGTTTGCAAGCTTTCAACAGGTAATATGATGT
AATTGGTTCCATTATAGTTTGAAGCTGTCACTGCTGCATGTTTATCTTGCCTATGCTGCTGTATCTTATT
CCTTCCACTGTTCAGAAGTCTAATATGGGAAGCCATATATCAGTGGTAAAGTGAAGCAAATTGTTCTACC
AAGACCTCATTCTTCATGTCATTAAGCAATAGGTTGCAGCAAACAAGGAAGAGCTTCTTGCTTTTTATTC
TTCCAACCTTAATTGAACACTCAATGATGAAAAGCCCGACTGTACAAACATGTTGCAAGCTGCTTAAATC
TGTTTAAAATATATGGTTAGAGTTTTCTAAGAAAATATAAATACTGTAAAAAGTTCATTTTATTTTATTT
TTCAGCCTTTTGTACGTAAAATGAGAAATTAAAAGTATCTTCAGGTGGATGTCACAGTCACTATTGTTAG
TTTCTGTTCCTAGCACTTTTAAATTGAAGCACTTCACAAAATAAGAAGCAAGGACTAGGATGCAGTGTAG
GTTTCTGCTTTTTTATTAGTACTGTAAACTTGCACACATTTCAATGTGAAACAAATCTCAAACTGAGTTC
AATGTTTATTTGCTTTCAATAGTAATGCCTTATCATTGAAAGAGGCTTAAAGAAAAAAAAAATCAGCTGA
TACTCTTGGCATTGCTTGAATCCAATGTTTCCACCTAGTCTTTTTATTCAGTAATCATCAGTCTTTTCCA
ATGTTTGTTTACACAGATAGATCTTATTGACCCATATGGCACTAGAACTGTATCAGATATAATATGGGAT
CCCAGCTTTTTTTCCTCTCCCACAAAACCAGGTAGTGAAGTTATATTACCAGTTACAGCAAAATACTTTG
TGTTTCACAAGCAACAATAAATGTAGATTCTTTATACTGAAGCTATTGACTTGTAGTGTGTTGGTGAAAT
GCATGCAGGAAAATGCTGTTACCATAAAGAACGGTAAACCACATTACAATCAAGCCAAAAGAATAAAGGT
TTCGCTTTTGTTTTTGTATTTAATTGTTGTCTTTGTTTCTATCTTTGAAATGCCATTTAAAGGTAGATTT
CTATCATGTAAAAATAATCTATCTGAAAAACAAATGTAAAGAACACACATTAATTACTATAATTCATCTT
TCAATTTTTTCATGGAATGGAAGTTAATTAAGAAGAGTGTATTGGATAACTACTTTAATATTGGCCAAAA
AGCTAGATATGGCATCAGGTAGACTAGTGGAAAGTTACAAAAATTAATAAAAAATTGACTAACA
SEQ ID NO: 9; SCN2A protein isoform 1 (NP_001358176.1):
MAQSVLVPPGPDSFRFFTRESLAAIEQRIAEEKAKRPKQERKDEDDENGPKPNSDLEAGKSLPFIYGDIP
PEMVSVPLEDLDPYYINKKTFIVLNKGKAISRFSATPALYILTPFNPIRKLAIKILVHSLFNMLIMCTIL
TNCVFMTMSNPPDWTKNVEYTFTGIYTFESLIKILARGFCLEDFTFLRDPWNWLDFTVITFAYVTEFVDL
GNVSALRTFRVLRALKTISVIPGLKTIVGALIQSVKKLSDVMILTVFCLSVFALIGLQLFMGNLRNKCLQ
WPPDNSSFEINITSFFNNSLDGNGTTFNRTVSIFNWDEYIEDKSHFYFLEGQNDALLCGNSSDAGQCPEG
YICVKAGRNPNYGYTSFDTFSWAFLSLFRLMTQDFWENLYQLTLRAAGKTYMIFFVLVIFLGSFYLINLI
LAVVAMAYEEQNQATLEEAEQKEAEFQQMLEQLKKQQEEAQAAAAAASAESRDFSGAGGIGVFSESSSVA
SKLSSKSEKELKNRRKKKKQKEQSGEEEKNDRVRKSESEDSIRRKGFRFSLEGSRLTYEKRFSSPHQSLL
SIRGSLFSPRRNSRASLFSFRGRAKDIGSENDFADDEHSTFEDNDSRRDSLFVPHRHGERRHSNVSQASR
ASRVLPILPMNGKMHSAVDCNGVVSLVGGPSTLTSAGQLLPEGTTTETEIRKRRSSSYHVSMDLLEDPTS
RQRAMSIASILTNTMEELEESRQKCPPCWYKFANMCLIWDCCKPWLKVKHLVNLVVMDPFVDLAITICIV
LNTLFMAMEHYPMTEQFSSVLSVGNLVFTGIFTAEMFLKIIAMDPYYYFQEGWNIFDGFIVSLSLMELGL
ANVEGLSVLRSFRLLRVFKLAKSWPTLNMLIKIIGNSVGALGNLTLVLAIIVFIFAVVGMQLFGKSYKEC
VCKISNDCELPRWHMHDFFHSFLIVFRVLCGEWIETMWDCMEVAGQTMCLTVFMMVMVIGNLVVLNLFLA
LLLSSFSSDNLAATDDDNEMNNLQIAVGRMQKGIDFVKRKIREFIQKAFVRKQKALDEIKPLEDLNNKKD
SCISNHTTIEIGKDLNYLKDGNGTTSGIGSSVEKYVVDESDYMSFINNPSLTVTVPIAVGESDFENLNTE
EFSSESDMEESKEKLNATSSSEGSTVDIGAPAEGEQPEVEPEESLEPEACFTEDCVRKFKCCQISIEEGK
GKLWWNLRKTCYKIVEHNWFETFIVFMILLSSGALAFEDIYIEQRKTIKTMLEYADKVFTYIFILEMLLK
WVAYGFQVYFTNAWCWLDFLIVDVSLVSLTANALGYSELGAIKSLRTLRALRPLRALSRFEGMRVVVNAL
LGAIPSIMNVLLVCLIFWLIFSIMGVNLFAGKFYHCINYTTGEMFDVSVVNNYSECKALIESNQTARWKN
VKVNFDNVGLGYLSLLQVATFKGWMDIMYAAVDSRNVELQPKYEDNLYMYLYFVIFIIFGSFFTLNLFIG
VIIDNFNQQKKKFGGQDIFMTEEQKKYYNAMKKLGSKKPQKPIPRPANKFQGMVFDFVTKQVFDISIMIL
ICLNMVTMMVETDDQSQEMTNILYWINLVFIVLFTGECVLKLISLRYYYFTIGWNIFDFVVVILSIVGMF
LAELIEKYFVSPTLFRVIRLARIGRILRLIKGAKGIRTLLFALMMSLPALFNIGLLLFLVMFIYAIRGMS
NFAYVKREVGIDDMFNFETFGNSMICLFQITTSAGWDGLLAPILNSGPPDCDPDKDHPGSSVKGDCGNPS
VGIFFFVSYIIISFLVVVNMYIAVILENFSVATEESAEPLSEDDFEMFYEVWEKFDPDATQFIEFAKLSD
FADALDPPLLIAKPNKVQLIAMDLPMVSGDRIHCLDILFAFTKRVLGESGEMDALRIQMEERFMASNPSK
VSYEPITTTLKRKQEEVSAIIIQRAYRRYLLKQKVKKVSSIYKKDKGKECDGTPIKEDTLIDKLNENSTP
EKTDMTPSTTSPPSYDSVTKPEKEKFEKDKSEKEDKGKDIRESKK
SEQ ID NO: 10; SCN2A transcript variant1 (NM_021007.3):
AAGCATGATGGAATTTTAGCTGCAGTCTTCTTGGTGCCAGCTTATCAATCCCAAACTCTGGGTGTAAAAG
ATTCTACAGGGCACTTTCTTATGCAAGGAGCTAAACAGTGATTAAAGGAGCAGGATGAAAAGATGGCACA
GTCAGTGCTGGTACCGCCAGGACCTGACAGCTTCCGCTTCTTTACCAGGGAATCCCTTGCTGCTATTGAA
CAACGCATTGCAGAAGAGAAAGCTAAGAGACCCAAACAGGAACGCAAGGATGAGGATGATGAAAATGGCC
CAAAGCCAAACAGTGACTTGGAAGCAGGAAAATCTCTTCCATTTATTTATGGAGACATTCCTCCAGAGAT
GGTGTCAGTGCCCCTGGAGGATCTGGACCCCTACTATATCAATAAGAAAACGTTTATAGTATTGAATAAA
GGGAAAGCAATCTCTCGATTCAGTGCCACCCCTGCCCTTTACATTTTAACTCCCTTCAACCCTATTAGAA
AATTAGCTATTAAGATTTTGGTACATTCTTTATTCAATATGCTCATTATGTGCACGATTCTTACCAACTG
TGTATTTATGACCATGAGTAACCCTCCAGACTGGACAAAGAATGTGGAGTATACCTTTACAGGAATTTAT
ACTTTTGAATCACTTATTAAAATACTTGCAAGGGGCTTTTGTTTAGAAGATTTCACATTTTTACGGGATC
CATGGAATTGGTTGGATTTCACAGTCATTACTTTTGCATATGTGACAGAGTTTGTGGACCTGGGCAATGT
CTCAGCGTTGAGAACATTCAGAGTTCTCCGAGCATTGAAAACAATTTCAGTCATTCCAGGCCTGAAGACC
ATTGTGGGGGCCCTGATCCAGTCAGTGAAGAAGCTTTCTGATGTCATGATCTTGACTGTGTTCTGTCTAA
GCGTGTTTGCGCTAATAGGATTGCAGTTGTTCATGGGCAACCTACGAAATAAATGTTTGCAATGGCCTCC
AGATAATTCTTCCTTTGAAATAAATATCACTTCCTTCTTTAACAATTCATTGGATGGGAATGGTACTACT
TTCAATAGGACAGTGAGCATATTTAACTGGGATGAATATATTGAGGATAAAAGTCACTTTTATTTTTTAG
AGGGGCAAAATGATGCTCTGCTTTGTGGCAACAGCTCAGATGCAGGCCAGTGTCCTGAAGGATACATCTG
TGTGAAGGCTGGTAGAAACCCCAACTATGGCTACACGAGCTTTGACACCTTTAGTTGGGCCTTTTTGTCC
TTATTTCGTCTCATGACTCAAGACTTCTGGGAAAACCTTTATCAACTGACACTACGTGCTGCTGGGAAAA
CGTACATGATATTTTTTGTGCTGGTCATTTTCTTGGGCTCATTCTATCTAATAAATTTGATCTTGGCTGT
GGTGGCCATGGCCTATGAGGAACAGAATCAGGCCACATTGGAAGAGGCTGAACAGAAGGAAGCTGAATTT
CAGCAGATGCTCGAACAGTTGAAAAAGCAACAAGAAGAAGCTCAGGCGGCAGCTGCAGCCGCATCTGCTG
AATCAAGAGACTTCAGTGGTGCTGGTGGGATAGGAGTTTTTTCAGAGAGTTCTTCAGTAGCATCTAAGTT
GAGCTCCAAAAGTGAAAAAGAGCTGAAAAACAGAAGAAAGAAAAAGAAACAGAAAGAACAGTCTGGAGAA
GAAGAGAAAAATGACAGAGTCCGAAAATCGGAATCTGAAGACAGCATAAGAAGAAAAGGTTTCCGTTTTT
CCTTGGAAGGAAGTAGGCTGACATATGAAAAGAGATTTTCTTCTCCACACCAGTCCTTACTGAGCATCCG
TGGCTCCCTTTTCTCTCCAAGACGCAACAGTAGGGCGAGCCTTTTCAGCTTCAGAGGTCGAGCAAAGGAC
ATTGGCTCTGAGAATGACTTTGCTGATGATGAGCACAGCACCTTTGAGGACAATGACAGCCGAAGAGACT
CTCTGTTCGTGCCGCACAGACATGGAGAACGGCGCCACAGCAATGTCAGCCAGGCCAGCCGTGCCTCCAG
GGTGCTCCCCATCCTGCCCATGAATGGGAAGATGCATAGCGCTGTGGACTGCAATGGTGTGGTCTCCCTG
GTCGGGGGCCCTTCTACCCTCACATCTGCTGGGCAGCTCCTACCAGAGGGCACAACTACTGAAACAGAAA
TAAGAAAGAGACGGTCCAGTTCTTATCATGTTTCCATGGATTTATTGGAAGATCCTACATCAAGGCAAAG
AGCAATGAGTATAGCCAGTATTTTGACCAACACCATGGAAGAACTTGAAGAATCCAGACAGAAATGCCCA
CCATGCTGGTATAAATTTGCTAATATGTGTTTGATTTGGGACTGTTGTAAACCATGGTTAAAGGTGAAAC
ACCTTGTCAACCTGGTTGTAATGGACCCATTTGTTGACCTGGCCATCACCATCTGCATTGTCTTAAATAC
ACTCTTCATGGCTATGGAGCACTATCCCATGACGGAGCAGTTCAGCAGTGTACTGTCTGTTGGAAACCTG
GTCTTCACAGGGATCTTCACAGCAGAAATGTTTCTCAAGATAATTGCCATGGATCCATATTATTACTTTC
AAGAAGGCTGGAATATTTTTGATGGTTTTATTGTGAGCCTTAGTTTAATGGAACTTGGTTTGGCAAATGT
GGAAGGATTGTCAGTTCTCCGATCATTCCGGCTGCTCCGAGTTTTCAAGTTGGCAAAATCTTGGCCAACT
CTAAATATGCTAATTAAGATCATTGGCAATTCTGTGGGGGCTCTAGGAAACCTCACCTTGGTATTGGCCA
TCATCGTCTTCATTTTTGCTGTGGTCGGCATGCAGCTCTTTGGTAAGAGCTACAAAGAATGTGTCTGCAA
GATTTCCAATGATTGTGAACTCCCACGCTGGCACATGCATGACTTTTTCCACTCCTTCCTGATCGTGTTC
CGCGTGCTGTGTGGAGAGTGGATAGAGACCATGTGGGACTGTATGGAGGTCGCTGGCCAAACCATGTGCC
TTACTGTCTTCATGATGGTCATGGTGATTGGAAATCTAGTGGTTCTGAACCTCTTCTTGGCCTTGCTTTT
GAGTTCCTTCAGTTCTGACAATCTTGCTGCCACTGATGATGATAACGAAATGAATAATCTCCAGATTGCT
GTGGGAAGGATGCAGAAAGGAATCGATTTTGTTAAAAGAAAAATACGTGAATTTATTCAGAAAGCCTTTG
TTAGGAAGCAGAAAGCTTTAGATGAAATTAAACCGCTTGAAGATCTAAATAATAAAAAAGACAGCTGTAT
TTCCAACCATACCACCATAGAAATAGGCAAAGACCTCAATTATCTCAAAGACGGAAATGGAACTACTAGT
GGCATAGGCAGCAGTGTAGAAAAATATGTCGTGGATGAAAGTGATTACATGTCATTTATAAACAACCCTA
GCCTCACTGTGACAGTACCAATTGCTGTTGGAGAATCTGACTTTGAAAATTTAAATACTGAAGAATTCAG
CAGCGAGTCAGATATGGAGGAAAGCAAAGAGAAGCTAAATGCAACTAGTTCATCTGAAGGCAGCACGGTT
GATATTGGAGCTCCCGCCGAGGGAGAACAGCCTGAGGTTGAACCTGAGGAATCCCTTGAACCTGAAGCCT
GTTTTACAGAAGACTGTGTACGGAAGTTCAAGTGTTGTCAGATAAGCATAGAAGAAGGCAAAGGGAAACT
CTGGTGGAATTTGAGGAAAACATGCTATAAGATAGTGGAGCACAATTGGTTCGAAACCTTCATTGTCTTC
ATGATTCTGCTGAGCAGTGGGGCTCTGGCCTTTGAAGATATATACATTGAGCAGCGAAAAACCATTAAGA
CCATGTTAGAATATGCTGACAAGGTTTTCACTTACATATTCATTCTGGAAATGCTGCTAAAGTGGGTTGC
ATATGGTTTTCAAGTGTATTTTACCAATGCCTGGTGCTGGCTAGACTTCCTGATTGTTGATGTCTCACTG
GTTAGCTTAACTGCAAATGCCTTGGGTTACTCAGAACTTGGTGCCATCAAATCCCTCAGAACACTAAGAG
CTCTGAGGCCACTGAGAGCTTTGTCCCGGTTTGAAGGAATGAGGGTTGTTGTAAATGCTCTTTTAGGAGC
CATTCCATCTATCATGAATGTACTTCTGGTTTGTCTGATCTTTTGGCTAATATTCAGTATCATGGGAGTG
AATCTCTTTGCTGGCAAGTTTTACCATTGTATTAATTACACCACTGGAGAGATGTTTGATGTAAGCGTGG
TCAACAACTACAGTGAGTGCAAAGCTCTCATTGAGAGCAATCAAACTGCCAGGTGGAAAAATGTGAAAGT
AAACTTTGATAACGTAGGACTTGGATATCTGTCTCTACTTCAAGTAGCCACGTTTAAGGGATGGATGGAT
ATTATGTATGCAGCTGTTGATTCACGAAATGTAGAATTACAACCCAAGTATGAAGACAACCTGTACATGT
ATCTTTATTTTGTCATCTTTATTATTTTTGGTTCATTCTTTACCTTGAATCTTTTCATTGGTGTCATCAT
AGATAACTTCAACCAACAGAAAAAGAAGTTTGGAGGTCAAGACATTTTTATGACAGAAGAACAGAAGAAA
TACTACAATGCAATGAAAAAACTGGGTTCAAAGAAACCACAAAAACCCATACCTCGACCTGCTAACAAAT
TCCAAGGAATGGTCTTTGATTTTGTAACCAAACAAGTCTTTGATATCAGCATCATGATCCTCATCTGCCT
TAACATGGTCACCATGATGGTGGAAACCGATGACCAGAGTCAAGAAATGACAAACATTCTGTACTGGATT
AATCTGGTGTTTATTGTTCTGTTCACTGGAGAATGTGTGCTGAAACTGATCTCTCTTCGTTACTACTATT
TCACTATTGGATGGAATATTTTTGATTTTGTGGTGGTCATTCTCTCCATTGTAGGAATGTTTCTGGCTGA
ACTGATAGAAAAGTATTTTGTGTCCCCTACCCTGTTCCGAGTGATCCGTCTTGCCAGGATTGGCCGAATC
CTACGTCTGATCAAAGGAGCAAAGGGGATCCGCACGCTGCTCTTTGCTTTGATGATGTCCCTTCCTGCGT
TGTTTAACATCGGCCTCCTTCTTTTCCTGGTCATGTTCATCTACGCCATCTTTGGGATGTCCAATTTTGC
CTATGTTAAGAGGGAAGTTGGGATCGATGACATGTTCAACTTTGAGACCTTTGGCAACAGCATGATCTGC
CTGTTCCAAATTACAACCTCTGCTGGCTGGGATGGATTGCTAGCACCTATTCTTAATAGTGGACCTCCAG
ACTGTGACCCTGACAAAGATCACCCTGGAAGCTCAGTTAAAGGAGACTGTGGGAACCCATCTGTTGGGAT
TTTCTTTTTTGTCAGTTACATCATCATATCCTTCCTGGTTGTGGTGAACATGTACATCGCGGTCATCCTG
GAGAACTTCAGTGTTGCTACTGAAGAAAGTGCAGAGCCTCTGAGTGAGGATGACTTTGAGATGTTCTATG
AGGTTTGGGAGAAGTTTGATCCCGATGCGACCCAGTTTATAGAGTTTGCCAAACTTTCTGATTTTGCAGA
TGCCCTGGATCCTCCTCTTCTCATAGCAAAACCCAACAAAGTCCAGCTCATTGCCATGGATCTGCCCATG
GTGAGTGGTGACCGGATCCACTGTCTTGACATCTTATTTGCTTTTACAAAGCGTGTTTTGGGTGAGAGTG
GAGAGATGGATGCCCTTCGAATACAGATGGAAGAGCGATTCATGGCATCAAACCCCTCCAAAGTCTCTTA
TGAGCCCATTACGACCACGTTGAAACGCAAACAAGAGGAGGTGTCTGCTATTATTATCCAGAGGGCTTAC
AGACGCTACCTCTTGAAGCAAAAAGTTAAAAAGGTATCAAGTATATACAAGAAAGACAAAGGCAAAGAAT
GTGATGGAACACCCATCAAAGAAGATACTCTCATTGATAAACTGAATGAGAATTCAACTCCAGAGAAAAC
CGATATGACGCCTTCCACCACGTCTCCACCCTCGTATGATAGTGTGACCAAACCAGAAAAAGAAAAATTT
GAAAAAGACAAATCAGAAAAGGAAGACAAAGGGAAAGATATCAGGGAAAGTAAAAAGTAAAAAGAAACCA
AGAATTTTCCATTTTGTGATCAATTGTTTACAGCCCGTGATGGTGATGTGTTTGTGTCAACAGGACTCCC
ACAGGAGGTCTATGCCAAACTGACTGTTTTTACAAATGTATACTTAAGGTCAGTGCCTATAACAAGACAG
AGACCTCTGGTCAGCAAACTGGAACTCAGTAAACTGGAGAAATAGTATCGATGGGAGGTTTCTATTTTCA
CAACCAGCTGACACTGCTGAAGAGCAGAGGCGTAATGGCTACTCAGACGATAGGAACCAATTTAAAGGGG
GGAGGGAAGTTAAATTTTTATGTAAATTCAACATGTGACACTTGATAATAGTAATTGTCACCAGTGTTTA
TGTTTTAACTGCCACACCTGCCATATTTTTACAAAACGTGTGCTGTGAATTTATCACTTTTCTTTTTAAT
TCACAGGTTGTTTACTATTATATGTGACTATTTTTGTAAATGGGTTTGTGTTTGGGGAGAGGGATTAAAG
GGAGGGAATTCTACATTTCTCTATTGTATTGTATAACTGGATATATTTTAAATGGAGGCATGCTGCAATT
CTCATTCACACATAAAAAAATCACATCACAAAAGGGAAGAGTTTACTTCTTGTTTCAGGATGTTTTTAGA
TTTTTGAGGTGCTTAAATAGCTATTCGTATTTTTAAGGTGTCTCATCCAGAAAAAATTTAATGTGCCTGT
AAATGTTCCATAGAATCACAAGCATTAAAGAGTTGTTTTATTTTTACATAACCCATTAAATGTACATGTA
TATATGTATATATGTATATGTGCGTGTATATACATATATATGTATACACACATGCACACACAGAGATATA
CACATACCATTACATTGTCATTCACAGTCCCAGCAGCATGACTATCACATTTTTGATAAGTGTCCTTTGG
CATAAAATAAAAATATCCTATCAGTCCTTTCTAAGAAGCCTGAATTGACCAAAAAACATCCCCACCACCA
CTTTATAAAGTTGATTCTGCTTTATCCTGCAGTATTGTTTAGCCATCTTCTGCTCTTGGTAAGGTTGACA
TAGTATATGTCAATTTAAAAAATAAAAGTCTGCTTTGTAAATAGTAATTTTACCCAGTGGTGCATGTTTG
AGCAAACAAAAATGATGATTTAAGCACACTACTTATTGCATCAAATATGTACCACAGTAAGTATAGTTTG
CAAGCTTTCAACAGGTAATATGATGTAATTGGTTCCATTATAGTTTGAAGCTGTCACTGCTGCATGTTTA
TCTTGCCTATGCTGCTGTATCTTATTCCTTCCACTGTTCAGAAGTCTAATATGGGAAGCCATATATCAGT
GGTAAAGTGAAGCAAATTGTTCTACCAAGACCTCATTCTTCATGTCATTAAGCAATAGGTTGCAGCAAAC
AAGGAAGAGCTTCTTGCTTTTTATTCTTCCAACCTTAATTGAACACTCAATGATGAAAAGCCCGACTGTA
CAAACATGTTGCAAGCTGCTTAAATCTGTTTAAAATATATGGTTAGAGTTTTCTAAGAAAATATAAATAC
TGTAAAAAGTTCATTTTATTTTATTTTTCAGCCTTTTGTACGTAAAATGAGAAATTAAAAGTATCTTCAG
GTGGATGTCACAGTCACTATTGTTAGTTTCTGTTCCTAGCACTTTTAAATTGAAGCACTTCACAAAATAA
GAAGCAAGGACTAGGATGCAGTGTAGGTTTCTGCTTTTTTATTAGTACTGTAAACTTGCACACATTTCAA
TGTGAAACAAATCTCAAACTGAGTTCAATGTTTATTTGCTTTCAATAGTAATGCCTTATCATTGAAAGAG
GCTTAAAGAAAAAAAAAATCAGCTGATACTCTTGGCATTGCTTGAATCCAATGTTTCCACCTAGTCTTTT
TATTCAGTAATCATCAGTCTTTTCCAATGTTTGTTTACACAGATAGATCTTATTGACCCATATGGCACTA
GAACTGTATCAGATATAATATGGGATCCCAGCTTTTTTTCCTCTCCCACAAAACCAGGTAGTGAAGTTAT
ATTACCAGTTACAGCAAAATACTTTGTGTTTCACAAGCAACAATAAATGTAGATTCTTTATACTGAAGCT
ATTGACTTGTAGTGTGTTGGTGAAATGCATGCAGGAAAATGCTGTTACCATAAAGAACGGTAAACCACAT
TACAATCAAGCCAAAAGAATAAAGGTTTCGCTTTTGTTTTTGTATTTAATTGTTGTCTTTGTTTCTATCT
TTGAAATGCCATTTAAAGGTAGATTTCTATCATGTAAAAATAATCTATCTGAAAAACAAATGTAAAGAAC
ACACATTAATTACTATAATTCATCTTTCAATTTTTTCATGGAATGGAAGTTAATTAAGAAGAGTGTATTG
GATAACTACTTTAATATTGGCCAAAAAGCTAGATATGGCATCAGGTAGACTAGTGGAAAGTTACAAAAAT
TAATAAAAAATTGACTAACA
SEQ ID NO: 11; SCN2A protein isoform 1 (NP_066287.2):
MAQSVLVPPGPDSFRFFTRESLAAIEQRIAEEKAKRPKQERKDEDDENGPKPNSDLEAGKSLPFIYGDIP
PEMVSVPLEDLDPYYINKKTFIVLNKGKAISRFSATPALYILTPFNPIRKLAIKILVHSLFNMLIMCTIL
TNCVFMTMSNPPDWTKNVEYTFTGIYTFESLIKILARGFCLEDFTFLRDPWNWLDFTVITFAYVTEFVDL
GNVSALRTFRVLRALKTISVIPGLKTIVGALIQSVKKLSDVMILTVFCLSVFALIGLQLFMGNLRNKCLQ
WPPDNSSFEINITSFFNNSLDGNGTTFNRTVSIFNWDEYIEDKSHFYFLEGQNDALLCGNSSDAGQCPEG
YICVKAGRNPNYGYTSFDTFSWAFLSLFRLMTQDFWENLYQLTLRAAGKTYMIFFVLVIFLGSFYLINLI
LAVVAMAYEEQNQATLEEAEQKEAEFQQMLEQLKKQQEEAQAAAAAASAESRDFSGAGGIGVFSESSSVA
SKLSSKSEKELKNRRKKKKQKEQSGEEEKNDRVRKSESEDSIRRKGFRFSLEGSRLTYEKRFSSPHQSLL
SIRGSLFSPRRNSRASLFSFRGRAKDIGSENDFADDEHSTFEDNDSRRDSLFVPHRHGERRHSNVSQASR
ASRVLPILPMNGKMHSAVDCNGVVSLVGGPSTLTSAGQLLPEGTTTETEIRKRRSSSYHVSMDLLEDPTS
RQRAMSIASILTNTMEELEESRQKCPPCWYKFANMCLIWDCCKPWLKVKHLVNLVVMDPFVDLAITICIV
LNTLFMAMEHYPMTEQFSSVLSVGNLVFTGIFTAEMFLKIIAMDPYYYFQEGWNIFDGFIVSLSLMELGL
ANVEGLSVLRSFRLLRVFKLAKSWPTLNMLIKIIGNSVGALGNLTLVLAIIVFIFAVVGMQLFGKSYKEC
VCKISNDCELPRWHMHDFFHSFLIVFRVLCGEWIETMWDCMEVAGQTMCLTVFMMVMVIGNLVVLNLFLA
LLLSSFSSDNLAATDDDNEMNNLQIAVGRMQKGIDFVKRKIREFIQKAFVRKQKALDEIKPLEDLNNKKD
SCISNHTTIEIGKDLNYLKDGNGTTSGIGSSVEKYVVDESDYMSFINNPSLTVTVPIAVGESDFENLNTE
EFSSESDMEESKEKLNATSSSEGSTVDIGAPAEGEQPEVEPEESLEPEACFTEDCVRKFKCCQISIEEGK
GKLWWNLRKTCYKIVEHNWFETFIVFMILLSSGALAFEDIYIEQRKTIKTMLEYADKVFTYIFILEMLLK
WVAYGFQVYFTNAWCWLDFLIVDVSLVSLTANALGYSELGAIKSLRTLRALRPLRALSRFEGMRVVVNAL
LGAIPSIMNVLLVCLIFWLIFSIMGVNLFAGKFYHCINYTTGEMFDVSVVNNYSECKALIESNOTARWKN
VKVNFDNVGLGYLSLLQVATFKGWMDIMYAAVDSRNVELQPKYEDNLYMYLYFVIFIIFGSFFTLNLFIG
VIIDNFNQQKKKFGGQDIFMTEEQKKYYNAMKKLGSKKPQKPIPRPANKFQGMVFDFVTKQVFDISIMIL
ICLNMVTMMVETDDQSQEMTNILYWINLVFIVLFTGECVLKLISLRYYYFTIGWNIFDFVVVILSIVGMF
LAELIEKYFVSPTLFRVIRLARIGRILRLIKGAKGIRTLLFALMMSLPALFNIGLLLFLVMFIYAIFGMS
NFAYVKREVGIDDMFNFETFGNSMICLFQITTSAGWDGLLAPILNSGPPDCDPDKDHPGSSVKGDCGNPS
VGIFFFVSYIIISFLVVVNMYIAVILENFSVATEESAEPLSEDDFEMFYEVWEKFDPDATQFIEFAKLSD
FADALDPPLLIAKPNKVQLIAMDLPMVSGDRIHCLDILFAFTKRVLGESGEMDALRIQMEERFMASNPSK
VSYEPITTTLKRKQEEVSAIIIQRAYRRYLLKQKVKKVSSIYKKDKGKECDGTPIKEDTLIDKLNENSTP
EKTDMTPSTTSPPSYDSVTKPEKEKFEKDKSEKEDKGKDIRESKK

1. A method for increasing levels of SCN2A protein in a cell, comprising contacting the cell with an antisense oligonucleotide that enhances splicing at a splice site of a retained intron in an intron-retaining SCN2A mRNA or pre-mRNA, wherein the retained intron is selected from among intron 1, 2, 3, 4, 5, 11, 13, 17 and 24 and wherein the antisense oligonucleotide comprises a sequence of nucleobases that is complementary to a target region in the SCN2A mRNA or pre-mRNA.

2-51. (canceled)


SEQUENCE LISTING (208 sequences)

SEQ ID NO: 1 - DNA, 8776 bp, Homo sapiens
aacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcagcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcatatgtgacagagtttgtggacctgggcaatgtctcagcgttgagaacattcagagttctccgagcattgaaaacaatttcagtcattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

SEQ ID NO: 2 - DNA, 159909 bp, Homo sapiens
aagacttccatctgccctccgtaggttcaactttaatgcttcctacagatgggcaacagggctatctgctcagtgcaacccccagaattccaattgtctgtgctgtctccactccataccataactaagcctaggaagcgcctcctgaagttcttcactgggctgaattaagaacaggatcatcgttctctatctgggtgttttctaccccactgagagaggtgaaattgtcagaacaaaaatttaggaatttaccaaatgcagtttttttttggctgataattactattattaacaataatgacaatgataagcttagaaaaattaaacactaaacaatggctgccgtaagaacactgaaatgactttatagtttacaatcagtctttcgtctttcagggatatttttgtggacatctggtgccacattttagtatggggttcagagaatatcaatttacctgtttcttcatcatgctatattgagttatttactccacaataaaggtagacctactgatgagaaatgtcagggcaacctttgtgtatttcagttaccctcagcaaggtgctaccagctagcattagtgtatttctggtgttggtaacaggatcatgtgagccttgtagatgagaactccttgttgctcaggggatcatggaatgctgctctctttcagacagccatcttcggggaagagaagggtctctggcagagctctatgggtcctgcacttctctttcacctctctgccttgttctttcactcattaatttcttgacaatttattttccttaataactctagttccttctgtttatcttcttctgtcaatccttgcatccattgcaaatccatcacctgctctgaaataccacactgaagggaggtactcctaaagaccttccttcttttccactcttgttttacccttggcttacacaactgttgaacaactggagctgaggacaaaaagcacataaattccaaaggtcaacggaatcacttcttcatagtttacatctcctgtgtcttaagtagaactcaaatagaacataagcattgagaacagaggtctgagttggaggtcaagacatcgtttactagctgagggactttggctttatttttctcatctctaagagggaaaagataaaaatctacttcacagacctgttgctgggaataatgagataatttatgtacagtacttagcacattgcttggcacataaaaagtgctcaataaatgttatatatttataacattatttgtaacacctcgtctttcaaggaattatgggaattattctgtttgtcagactttgtttatggaaattatgtccttttgatggcccaatttttcttttccttttcttcatcttttgataacatactattaaatgtcaacaattcatactaattggaactagtgtaataagctgtcctcctcatcagtattagcagtctttgaacatggcataaaaatatgcccattttggcttacaggtccttgattctctaatgataatagtgcaggaaaaacattagatatggagttcaactttttaaaactgtcttgatgttggtgaccaataatttaaaagaggttagcatattttaaggtgactaaaactgtacaaatgaaattgccatttaaaaatgtctgttcatttgcttttctccaatgcagatattttaaataagaagaagtgaggcaagaaaaatgtaaacaaatattaggtataaactaggaacttagtcttttattttctgaatcagatggatgatatttaaaagtctgggtgaatgataaatggtagaaaaaaattaaataacagtcaatatgcacaacttaatggaggtaaaataagacttacttacttttatctctataaattaaaatgattttgaaaatcaagctgccaaaataaaggtgtggattttatcattaaagcaatgcccaagagacatgttattgagaaattatgtggcattggtttgttgttactagcgaaatgaatgcctaactgaagttgatttcataaagatttagtgtgtgtatgtacttgtgtgtctgtgtgtatatatgtatatatatatttgtatacacacgcacacatgtctaccatgtgacatgttgctatttttacaacttattttaaaacagctaaaattttctagatgagattaaacattttccataaaaataaaattacaatagttacctcaaagtttgtttatcctggaaaaaaaagtgctaacataagagttgttcaataatattttaccaaaattttgtcagaactccattaaaaacaattcaagaccacagtggtctaatttcaacactggcatcattaacctaacatatttatggaatattcactatatgttctgttttctgcttaagtatatgaattaaacatagtttaaatagtttactcaaagaacatgccatttgcttcattgatttcacactctctgacgtatgtattaataagaacataggaatccacacacagaaataatagcattcctatggtgcattaataaaaatgtatgaattttctatgtctaggacatgtattcattatttgggtgggtcttatatttcaggtactgttctgtgaaccacgtttatggaagtgatcaaaacaggcaaacatctctgccttcatggaatttacatttttcttggaggaggcagacaatagacaaaatatttagtatgtcagctaaatactaaagggaaaaaccagaggaggggatagtgtgtggtattgagggtcaaggccagtctcagtaagaaggcagctgtgagctgtgacaagaatgagggaagtgagccatacggagcccatgtaagagatttccaggcaaagagattaaaaggtggacgcctcctaaggaatggacatacctgccatgtttgagtaatgagaaagaagataaaatggctaaagtagagtgagtgagggcaagaataacatcagagaagtaaaggagtctagattgtgagcattgtgaacactggggaaggcatagactattcacaaggagatgggtagctactaaaaagttttgagaagaagagtatgtgatatgatttagggttgaaaaggattgctccagtttctgtgtcatataaccctatggtcttgaaactgggatattgtatccctgggggcatgaggtggcgtgcctagaggtacacaaaatcccaggatcagttagtcacatcttccctaaggataaaatttaccctcaagactatggggaaaaatgattactgtgttaagaaaaacatttaaattttatataaattattgtaaaattcccgtgattttaaaaataaagtatcaataatatttcatatttgtggcatcttaggctttaatcttacaggtttaagcttcttggccatcaggagtaatttgttgaaattgcaaattaatttggcaaaattatcataattatgtgccctgagagcttatgtatcttatgatagtgcaggaaacttgtgagatgaaatagttgagtgtatacattaaaataatttgatctaccaagtcactttaattatttatttgttcagtgttaaaaatatattttcataaccaaactaatacatcttaataatgttgtagaaatggaagtaaggtaaaatttaagaaactgggtttgttcttctgacttgcttattaatactggataagttactgaagagtgggctttggtttcctcgtctatttaatagtgtgggaacaaccttatgttaaagcaatctattcagtataactttagcacacagaaaataaaatattatgaatccaaagaaagtgtagcttgcaatcatgtattttattaaatgtatttatcagcattttggcttctgatggacatgtgtatatacatattttgtggataaaaaaattacttaactatttttatattagatactatcaattattctggctgatctggggacttttaatggtaaattttatatcaaatctgacaaagacagtatttcagacccgtttaagacctagaactcaccatgagttctaaaattggttctcagcaccatggacagcgttactgcaataggaaattaaagatcgatttggccccaaattaaaatggtgttgtaaaaaagtgggagaaaaaaaatgcctatccttttacttcaaattttaaaaaaatgatcctggccttcacaactgttcataagaagaataattaattaaacaaacatatattgagaacatcatatgctcagtaaaactttgattctataaatggtgtcatttaccaaatggattcttttgacaatttaattttctcttatctctctaagaagatgtaactacacactatagtatactactacaattatcaaatttcatgttgcatgtaacttgtcgtctgtatttttgtagttagattagattaactaaagatttttcaagtttgcctttaagtcatttaattttcctgccttatctttaacctttcaacattcctccaaacaatagcaacacaagtgttatgtgttaacttctctagtgacaaaaacttatacttctccacaaagagatgtgatgttcattatcaataagcttgacatctaaaattgttttataggagatacatattactttttcagatggtatataaagttaaataaatcttaagttttcaatgatgggaaaagcttccatttagtttaaacataatgtaaagaaatttgaatccccaaaatagaattataattctaaaaattcatactataattcttcttaaatgtttaaattacagttaattaaagtagttgatttcaaatagagtggaattatgggctgtacatcatttaattttatgtgctgactgctacatagccaaaggaacgtgaattaagatggttccacttttgaccaggagatggagctgtcatgtaagatgctgcctttatttatttatttttctaatttagcatgctgttttctaacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcaggtaagccagcatgattctatttttgacttatccacggattgttatctatgttaagaatgacatttaatataagatgtgtgctttgttagcttgtattcagatctaagagattcaaaagctctaattctagctgttgtgcaatttaaaatcttcctaggctgaaatgagctctgactatgacataccgtgttttattatttctttggctttctagctgttggtctctgtctctggctgtatttgtttaccttttaaaggtaaagctttcaaagtgaaggatgggttttcctttcacaagggaaatactgctccttaatgcagtgaacaaattgtagagatataatgaagcagattaacatggcagtggctctgccaaagctcagggctgagccttctgattgcagcctcagaggcaagccaatattctgagtccaaaggccttttgaacgatttctgctggctatgatggtacagattgtttatgtctttatttttacctcttagaaataatacgtaagtcatttctcccacatgaatcaagatttatgtaatgtgtttaatagacttggaatgagactatagaccctccctcctcccccagtatggatgtgaggagtatgctgaacacaaattaattgccatgaaaattgattttttctgtattttattggatttgggataatagaaccgtggccattttaaatagttgatattatagaaggtgctcacattttaaattattttaggtcatgtgacattatctgtgttaaagagtggaatgactcaggaggtggaagttgcatgcaacagccttgcttctctctgctgcttctcgtgatttcccttaaatccttgcttgattattattagaataacaggaataaagacatatagaaatcctatatttaaaaaccactgattttcatttagagaaatttaaagagattttacaaatattttaaaaatagttttgtaagattgtttttaaatgcctcacttctttctcctgtggaaagagctgtgtgtgtgtgtgtgtgtgtgtgtgtgtgtgtgtgatggtggaggtggcaggcggtggaggtgcatcaggatgtttttatctcagtcattgttttcctgtaaagaggtagttttgccatgtcagaggttgtgaaagctatggggaatgattttataaaatggtttggacaaaccatgaaaaaaacatgttttttaaatgtgtggaatctatagagtttgtagttctacatggctccatatacatggagatttttagaattacagatatttaaccatttatccccattatcatttaaagaaaagcaattgtaattgagagataaggtggatttcccagagtcacgtactaaattacaataaggcaaggctagaacagtgattgacagttccacatcttccatagcagtaacacaattcacctctagtgtgaacatatcaggatggcatagaccaggtatgatgatattcactgtgcgtgtgtgtgtgtgtgtgtgtgtgcatgtgtgtgtgtgtttggttatattctgtaaaatagagtttctttcagtatactgttgctcaatggcatattatatgcagtgctcaattttcttaggtttcttgtcaaagcatccaagaaaactgaatgacaactatggaaactctgtttcttagtatgcctttttcattattaggtggtgtatactcttctaccctatgctcatgaatgatgtggatggccacatggctttccctcctacctaatagaaaagattgtacatataggttaatctattgacaactataagaatatcatctcatgtccaagcctttcctcctttgttcacttactaggcacaggctttaccctacttcttcaatatcttgagaaaagaaagggattaaaatgtgtcatgtctacatatatttgtggaataccacgtaccaaccaagtgtccatggtagagataaagaaataattagtcaaggagtttacctcagggaggttaacatgcaaatatacatgtgcagataccactatatctcaaaataaggaatgaagaaagatgagaaagtgaatgggacaggaatatcagcacctatcttgtgccaggcaccatgttacatcctgggattcagaacaaaacaataatgttaatttctgtccatggggggctcagaggaaaagattttaggaggatgtgacagttaaattagatctttaagagtgagttggatataaataaagaaaaacagaaagttgcttattccaggtacataggtgaggcaagtaaatacaagaaaatggagaactataggcatcgtatagggaaactgtagagaaaggtaaataacccaatctgtttgaagcttttgttgtcggtgttgttgttgcagttgttattttttttcccctggggcagtaaaagtaaggataatgaatttggcttttggtgttatgcatttgacatactgacacatactcatacactggggtgttttataaggagctgaacatgttatatccatgtatcagattagtggttaaggctgaatgtatagatctgcatgcagttgtgggacaaaattaggtcatacataattaaacattaatggggaacaaaagagaagtgaagatgactgggggcataatcttaagattaggagagtaaaaaagtgtagaaaactattattaaattgtgtgaaaataatgtgagaagttgacggaataacaatatcataggaggccaagcaggtaaattgaatatcaaaatagggatcagaaacaatagaaaacccataaaggggttggttcataagaggatagagtaaagatgatggatttagttatcattattaggtcataaattaattttaagagaagaggtttagttgaatggcagagacaggggctctgaggactaaattggacatgagaaatctggggagtatatttaagttttaagaagtttgccagggaaaggtaggagagataacattttatacctggagaaagaaggtactgttgagggaaatttaactttagtgcagattagaaaaacctaagccctttgtagagtaaggtatgaagtcagtagagagaaaattgaggaaagaagaaacaaaagtacaccaaggtcgtggaagagacagaaggaaggtttgaaaatgcttttccttgacacaggaagaaagggtggttgatgataatgagcaaagctggtgtaactcactttgggtggcctgtttttcgaaattaaatccttgtttgtaagatgaagtatctcctctgattaaatcagtttgttcctcagtaaaggctattggtggcatatggactttctggaagaaataattaatcctcagtagcaaaacactttcctgcagaatatattttagtcacattaattcttgaattggagactaataacactgtttcacatgaagcttttacatacttcagttatcaatttcttattcataaccacctatttaaaaacaggttctatttaaaacttgtattgaattaggccaacacatatctattatacatatctagtttgattactaagggctggtttgccagtatctaaattaattattcaagttctttccttttttgaatccttcagtttcattagcacgttcaccaggacttgtgtagagagagtcaatgtcatggccgggtgcagtgacttatgcctttactcccagcactttgggaggccgaggtgggcggattgcctgaggtctggagtttgagaccagcctggacaacatgctgaaaccctgtctctactaaaaataaaaaattagctgggtgtggtggcaggcacctgtagtcccagctactcgggaggctgaggcaggagaatcgcttgaacctgggaggcggaggttgcagtgagctgagattgcgccactgcactccagtgtgggtgacaaagtgagactctgttaaaaaaaaaaaaaaaaagtcaatgtcaggaagctcctttcccttagggaactagcttccttatgaagcttcctaccagaagttccttttttctccatttcttctggcagttacagaatcctcttggggctttcttagagccaatctcctctaaggtgaatgcattttcttgcattcacctgtcatgaaatggcagtggaaagacttgaagaaggcacaggcgttggtgagtcatatatatattttgctgataataatgaataccattggcaatataatagtggccattgaaatattatttctattaggaaaagataacttgcatcaagaatatgagatgaaattctattaaaaaatgaaaagtagatgggccaccgtggctcttgcttataatccacttcgggaggtcgaggtaggcagatcacctgaggtcaggagttctagacaagcctggccaatatggtgaaaacctgtctctagtaaaaatacaaaaattggccaggtgtggtggctgacgcctgtaatcccagctactccagaggccggggcaggagaatcacttaaacccaggaggcggaggttgcagtggactgagatcgtgccactgcactccagcttgggcgatagagggaaactctgagtcaaaaataaataaataaataaatagaaaaggaaaggaaagaagaaaaataaaagctgacctctctataacactcatacgttagtgttatctcctctttatccatttgggtttagtgaaattggaggtcagtggaaggagaggtagaagtggattttgcaaatcagaagtataatttttttttctggtaatgttccattaaacttttcagtcaaaacctttatgtggaagaagacataattactaactacattaataataataatgaaaaaatcttatcatgtaatatcgttttgtttattagtgaaacattttagagaaatgatttaagaatcaaaggtatttaagtatattttatttattggaatgtgataaaatatttttgagggtaaaaaactataatttcagttaactgtttgtacttactaaaataacatttagggggataaaatgatcttaggaagaaattgcccacaaaaagtgttttgaattgtttagattatttttatagttttaagttgttaagtcttttgctggtatctaggattagttgggagtataatatgctttgaacatgaacatgtcttggtgttattagcaaataggtatacaattatgtgattgtgtaattactcagctacatcttgtgttgtgtgatatgtatggggaaatgacaacaaaataacaacagttctcttaagcatatatgaaggtagtgcttgaaatatcatgaaaaaaagaaagggagattatgataatgttatgataagagaagactgacactatccctcttctctgaataactgatatcaaaatgtgaaattctaaggaatgaagcaagctggtcagggcaatggtgatgctgtctctgtatcagcccagcatttccagctaggatattttccttcttttctgtgtgatacagtttggctctgtatcctcacccaaatctcaccttgaattgtagttcccataatctccacgtgttgtgggagggaccctgtgggagataatttaatcacgggggtggttaccctcctgctgttcttgtgatagtgagtgagttctcactcgatctgatggttttataggggtttttctccctttgcttgacacttctctgtcctgacaccttgagaagaaggatgtgtttgcttacccttccgccatgattgtaaatttcctgaggccttcccagccatgcagcactgtgagtcaattaaacctctttcctttataaattacccattcttgggtatttcttcacagcagcatgagaactgactaacacactatgtctcccctggattgagcaaatgagggagcccagcaattgtcaggcagctaggatctgtgaggagtgcttagacctacacttactatttcctgaattaacagtctagaagacaggagcctagttatacagacagggtgaccataaaatgtgtcatctaaaccaggattcagtttgttaataatttctccacaacgacaggtataaatcaagatggttccaggtaaactgggatgcacagtcactccacttctaagggtcaagaaacttctacatttaatcagtctattcaattaaattgttcatgattttgttgcctaagtgttagaaagttaaaatgtgaaatgacctatcaattagacaaagggcattcagctaagctgctccaattactggccttgataaatccatcaaccgaaatctctaatgattcttgttgaatccttagttatatcaccaggaatgatgtcactccaataatgttaagcctgacttcccagggaattcaactgtgtgcaaaaaaaaactgttagtaaattatgctgttattacaaacctagactcacagcatcataaatctattcaaatataaaagcaaaagcaggataaaatatggattcaaattatttccacatttgggtatgcatcttttaaggagttttcatgaatgtgtaatccttagaagataggaatttagctccaagaacactggaattttctccactggcactttatacccaaatcttggtaatgtcaatatgcatatgtatggcctcttaacagactatcctgtaaagtagttaaattattttcctccaatgatcatgttttttttacccttcttcagacatccactcccatggttttatcttgaaactcttcatggccagtaactgtggcctgcccataatccttccttcatacatcctacaccacctttctagcgcatttcatctacaactccaattcattcctttaatcccaccaggcctctaaatcactgatctcgtcatactttgattatctctcactaaatgtcctctttcctttcattagataaattccacagttattcataaaagtcactccttgtatatacatacatacctcacaaactcctccgctcagtgtttttttgtttggttggttttttggtaactgtctggctaaatcttcatcctcatattcagttcccaccttacttcctgcctgcacttttgcagctggacgcatgcaaaagtgggtccttcatgatgcccagcaatcatgccacatgtccctagattgatgcccagcaaccatgccacatgtccctggatcacttactctcccaggttcttgtacttagatttcataccctttcttttctcctcaaaccaccaatacctcctaccttgacctcactctcagttgacaactttgtttcctaaatcagtgagaaaattgaaacaatcaggagagatttttccccacaggtttctatcactgcatgtgccttcctaccaggttctgcacccacataacctgacctcccacccattaccttaaatcaactatccctgtttctatctaaaatcattcctgcatctgtgcactagatcctattctttcttacttacacaagggcatctctagagcattttatttctctgtctctcctgcattagtaattttagctctttattatatttgcatacaagcattttattattgctcttgtatttgaatttttttacttccttcaactatcttttttttattttttgttgtctttgcaataatacttaatgaaagtgtttgctgtattctctgtatctatgactctctttctattctcttttaaatttacgttagtcaagtttttactcttaccatttcaccaaatcagctcctgatagaaatctgatgaccaccctattgccaaattcatcagtccatcctcagctttcatcttacttgaccatccagcaatattggagacagttcattgctgtctcccccttgatacactgccttccgttggatttcaggatagcacacaattttgactcttctcttagcttattgttcactccttcacagtcactttactctttactctgccttcccagacttcattatttgggtgtccctgattctgtccctgattttcttttcttctttatctactttcttttgatcccattgagttttatggcgttaaatgccatataaatcatgatgacatccaaatatttacctccagttttctgccaaactgaacttgtgtgtctaactgcttatttggcttggatatctagaagacactgaaaagttaacatccctaaactaaatttcggatagttcttcccaaccaacctgctccaatcctcgctttccccaccttacttttttcaggagataactctagttttcctcatcctcaggctaacaactctagtatcatccttgattattctttttctcttatatctgacattcagtccatgagaaattcctgttgatttcaacttcagaatatacccaggatctatgtgctgccaccttcacactgctaccctgctcaagccaccatcaagtttctcatggatttctgccttaatttcctaattggactttattcttctacttctccttacaatctattctcaatacggcaagacagaagtaagattacgccacatccctgctcaaaattctgcaagtgatatccagttttctcagggtaaaaggcacctgacaatttccaaaaagtctctatttagcctggtctccatcccattacagtatttctctgaatttgtctccagtttttctttccctcattcactttcactatttcttgaatattgcaggaacacttcaatcctttgggctgtttcctctgcctagaaaccttttgcctcaggcacttgcatgactaacctcctcacctgtttaaaatctgagctcaaatgtcgtttctaaataatacctactcttaactatcccatttaaaaatctcagcccccttcctctaacttcattcttatcgtctcccacattcttctatactttcttttctttgttatcatttgctttgctttgttctttttttccttttctttcctttcttttatttccttcccttctaacacatcacattatagcatactgtatacattgcatttctcttgttggtaatttaagccccaaatgggcagagattatcatctgtttggtgcactgatgtttcacaaatgcttagaacacttcctgatctatagaagagactaaaaatgtttgtagaataaaataatgaatcttagaaacttaaatttattcctttaagcatactaaggggcaccatgctttctctttcactttattgggctgattcacatttacaaatgacccagtgcaatctgcctgatttttcattcagtatttacaccaatagaaataaaatgcatattagtgttataaaatcaaaccttaaatgacagtaaatgtatcattaatagttactgaagaccttttcctgatatatatgacatttaatgtttttctcttgtgacttcattgttctttgtttgcttgttcatcttctagattgtgagctcctcaaagtcaggaacttggtcatcatatctgtctcccaaataccacactcaatgtctggcacataagaagttttacaaatatgctgttttagtcataggatgagcatacacttaaaatttaatactggtcctgcatatgctgtgtcctgagctttcgctatgtaggttgctttaattcttacaaccgtgtggagcaaagcactggtctcattttacagatgtgataaagctagttattttgtccaggacttaatcattgcagtattctcactgtctaatctctagtgaccatatcacaggaagttaattatgctctttttttaaagataaattgaaataatgctaacaatgccagatttcttggaaatgttttgtttttagtttcagttagaagcacgccttagcttagtgaataagggacaatgaatgttatttattttcctgctcaattcagttgggaaaattgacctcatcgttttgtgttttcaatcccttaagtgtaataaaattatttactaccttagtttatataaatcaccaagatctaatggaagccacccagttaactctcctaaagagtcttcaagcatgttttgttctaaatctcagcttaagtattttccattgtttaccacctaatgtctatactacttgatttatagaaagtcttatctaatataaatatgctaactagctatgataaaattgatcactttccacataatcatatactgaaaaaaaattccactgaatatatttctgccctccatagattccaggatttcagtgaatttttcttaataatctctgcttcagaaatgtatataaacttttgtcctaatcaaatctaactcaaacgtcacttcctttataatgttctctctgttttcacatgcagcatatacttccattctactctgcacctatctctacacttgtaatgtgaagtagccagatatatgtggacctgggtcatctgtcatatttaattgtgtactatataaggacagatattgttttgtatggttctatcaagctacccagtatctacatatactgccctccataggaggtaatacatatgctgagctctactcatttaaaacttcaatcaaggtactattggaatttcttagttttacaacataactatgtcctacttcaagtgacctattaaaatagctctcaatagtttgctttaatacaactgcctttaatagcttttaaatttagtgtttgtttgagtagactttatggtgatttgtaaaatgatatggatatagtatatagaaaattttaacagaaatgtaaataataattataatttagggaaacggtgccctcttgctctatttcacacacacacacacacacacacacacacacaggcacacatatacacacaaaaaaaatctctatttccaaatcttgttcacaaggagctctctaactccatgagtaatttttacaaccaataacagattaaccaacaatagtcacacgaacatgtacacacacatatacacagcctaacttccccacatcattattatctatctatcgtcaagaatgcagcttataaaatacaatgacctggctgctataggaaaggataaaatgtaggttgcagtgatcttctgacattgagatggcaaggaatgtgttttttgcaggaatttcaaaacctgaattgtagaatgatttgagatactgccttaactaccaacaaatttgatcccatgttcttcatcatttttctgagattctggtttcccagatttctgaccttaattgactcaaccaaaaaaatctgcttattattaaagaccataaggagtaatcaacagaacaaaatatgatttaatataatattaaatatattttacaactcaaaacattctaaaagatgtgatggtttctgactataattcagtatatactattttagttttagtgacaatgtttctaacaaatacaggaaaacgtttcagaacctagtgtcctttacagcaagtagcaaagatggactcaggaatatgtaagcaaaatctagccaaaccaaaccaaccaaccagacaacaacaacaaagtgtgttgctgagtggctcatagacaaggacaagtatttgtgatgttctaatataataggtggtaagaaccaatggaacatctgtgattcaagtgagagtcgtggtggtagaaaacaaaacaaaagaaacgataatatgtgtagaaataataaaagaaaaaagtcaaagggtgtatttttacaagtacacgttcaaatatagcgtgatgatattttatctcaggaatttcatgcaaagtgctgcgtatgttgatatgttaggtctcttctggcccagtgacgattatcttgtgtggtttaagttctctatgccatgagtgaggtaagggcatttattccccagatccttccctgccagattactgtggttgaatgtaaacaccacaactcctgccaagtggcccctactccctctgattctaattattatttcctctttttgcctcctgctgctgctagccttggggtgcactctgctttgtttcttttccttaaccctaatatacctttgtaaataatccttttttgttttaatctctcaattaaaatctcctcttggggtgtgtcatctgttttctgctaagactttgattaatataatagctaacatttatgaaatatttactataatacaggcactgttggaataattcacatggatccaatttttttatcctcacaacgatcatgtgaattaggtactattatgactaaatttaaaatataaggctactgaagaacatagattttaaataccttcatgatcatatagctaaaaactaagacaaaataaaatccaggagtctgactccagagcagagcttagatgtgaaagtaattaatatgcttttaaagaaaatcttatattgttggtgtctgtataaatcccatagacaagatctaagtacgaaaaaaaaatagaagcaagtcaggaaaacttagttcacaggtaataaaactagacacagatatctaaatttagaaatatcccatgcatatattctgcatatctgaatgcatcaagatattaataagcatcttaatactttggaaaaagtatagaggggactttctgtcaacatttgaagaaaacatttaactagtataaatatatcatacaagttaactgaaaaatctatctgctgtctatcctcatgttcagttatcaagtatatatcatttaaatttaagatgaacaaaacactacttaaatcccaaagactcattaaagtaaaactagtaaatgcattttatttatcaattatttccctgaaaatgcacattctttttaaattaattttttatttaaaaaagagcatcatgccattaaacaaaaaaaggaaaattttatttactgggaaattcttgctgatttttacatgggattgtgacaaaaaaaaggtcagatttttatttttattcacagtaaggagagattttatcttcagggatgattacaaggtggggaaagggcaaggatctcaagagttaggcaaagatttctcttttagagagaggagtaaacaaggctagaatgaactggatggggagaagagggatttaagggtggtatgatctgacagagaatgttccactctgagaccagcctgctaccttgagggactatgtaaggggagggggggtgtggctgcttcatcttgaaagtaagtcaaaattcagggatttggggaaagtagaaaatcttaaccaaaatttggttaataggcattttgttcccattggtcagtggggacgggcagtttagcttatcatttatgaggtaaagaatgggaattttgaagttctgtgttgggtctcatcacaggtgaatgggggacatttgtaagttttgcctgtgctatatggggaagggtcattctttacagtaatttcctagaacatgaaaggataggggggttcttattatttcatctacataaactggaacagaagatgatttcactggtttattaaactcctcaggatcttctacctgcccttcttaacctttacattccaaattcacttctatctcctttctagagctggtaatctttcttccatgaagtttatgaaattcttcatgacatgcatagactttgacagttctgtttctgatgtgcataatacctaccagattgatttgtgataatatcattgagaggccacttatagcctatttatgactcagcaaaccatggtcttttacagcctcttttaagatggtttggagagataaagaataatacttcatgacatgtaaaaattatattaagtttgaagtttaatgtccataaataagttttattgggacacagccatgcttatttttatataaattttccatgactgttttcttcctacaaggtgagaactgtatacttgcaacagagacaatatggcccacaaagccaaaattatttactaaatcatcccttgtagaaactaagttgcagactctggtctatatagtcacattacactacaatgatcaagtatagatgtttgacagtatttaagtttctgaatgaatgaacctatatagagaaatgtataggtataactatattactgatatatcctattaattttattatttttctgttgagtttttagcttttctaggtacataattttgtctgaaaataattaggacaattatgtcatgcctttctttgctaatatttctaagcaatcattcctttacttatttctaactaatgtttacccccagttatattgtcagattttcctggaaaatattaatagtgcaaatttcctgctgtttcctaactttgacataaatatttatagtggctcttcactaaattacatttacatcaatatccttagtgatatattgatgtaaatttgatttgagacagatcaacttcataatgtaaggatatttctctttatgaagagaatttattataatttataaataaatgtgatatctgttagatgacttttaagcatctagatgtagttcatattctgttttggcccattaatgtcatgtatttcattaatatatttttaagtactgcaccattttttcattactattttggatcttattttggggcattatgttattaagtagttgtatgttttatttaagaagtttataactgtattcattaatgatattggctgtagatttcatttttgagggtgttctttcggaaaagtttactatcaaatttatatttgcttcaaaaatatttttgagtaattttcctatttttctatcctttgaatgtgtttacaaaatgtggaaataagttgtttctagataatatcaatatttatttgctagaaaataaaaactcacctgtaaacatgttttattgttattttaaagtggttttgcatatttcttttaactcatttccttattaatttcctgatcaagttttcacttcttacatcattttgacaatttcactttcatagacgtacacattttgtagaaaaattcaaagattttaacatagttaataattttgtttgcatacttttcatctctcaaaagctaagaaaaatgaattcatgtcttctacaattaacagctttgcataataacgtactactattaactttatgattttatgaccagtatatatttattagaacatttacaagtacaaaattaccttacttatccatttaagttatttgtgttgagatatgttttctctctaataacattggaaaacctaaatttttgtttgtatttgttgatatatttttgtccattttttatttttaattttggcaatctgttctatgcttctttcaaataaacatctgttcaacttttgctttgtgatctaatttggtactttgtgttggttaattataaattaaccaattcaaaatttattattttaaatatcagataatatttgatttttcttctcataacttgttttgtggtttctacttttcttatatttttcttgattttcccataggggtatatcaactgcagtttttcccatctttattttttcttcttctattttttctagctttttggaatgcatgtattgccttatttcattccattattttaaaagaaaccatcagcctcataaatgaaactatctattaactacccttacataagataaaaatattgttttttctttcccttccaactattttccagtttaaaaaaatgtacatgatttggtattcacgttaatttaaaaatgttctccataagcgtattattttataaccttactaaccacgattattaaaatatggctctatggtttaactccatatcctaacaattattaaaatatggctctatggtttaactccatatcctaacaattattaaaatatggctctatggtttaaccccatatcctaactaaggatgttaccattattaccatgtattaccatttcttctagcatcttctgtagagctgaagtgtatcttagagtcacctcttcatgaaggagatattgatgatatattttctcagctgctggtagatgaaaatatttctgtaatgccctcatgtaataatgataatttcagttgatacgcaaatatttttcttaaaaatatattttgttcttttatcctcagacatctaatatgcagaagataagatacagccagatttttcttccccgcccaccccccttggaaacaaaccattttcccctaccagaaacttgatggatgtttttctttatcctttgaccttctctagctcaacaggaaatgttttcatttggggctcttttcttttttttttttttttttttggtgatggagtctctcgctctgtcgcccaggctggagtgcagtggcgcagtctcggctcactacaacctccgcctcccgggttcaaacaattctcctgttttagcctcctgagtagctgggactacaggcacctgcccccacggccgactaattatgtatttttagtagagatgggatttcaccttgttggtcaggctgatcgcaaactcctgacctcaggtgatccacccacctcggcctcccaaagtgctgggattacaggggtgagccactgcacctggcctattttctttaatgtaaatactattccataatcctgtttgatttacagactctttttgttaaaagatttcatttgttcttgagattagaattcgttgcaacaactgtggtagccttagtttttccttttcaatttactatctttaatgatagcatcctcagggcttttgcaccatgtatatctttacattcttaataattatttccatctaattaactttttactctgagatattgggaagttatcacagacttccatttaacaaacttaatttctgcattttcaaatatgcttattactgtttatactgaacttatgaatttaactatttttacaggtatccacgctatatccaactggactcccttgctaactgtcagctttcctttcataattaaagtgtcctctttaatcttaatcagagtatatcctaaaattaaaatgttctcttctgcttcttattttgaacaatggtatcttttcatatgtttggcgattttttttcctgcttactcatcattacagaagatactctatgctgtataacatttgtattttagatgattctatcaaatattgcttatttcctgtaaacatctaaaagtcttttaggctgaggagaagctataactttgttttaacagttactagttcaacttgtaacaaagttaaaacttttttttttaactttgctcatttaaatatacagtggcctaaggaaactaagaaccaatcacagcagagtttgtgtaggtttgatggctaggaccaaccatgggaactgacgcatcatttcctgaatttagttgcgttcaagggtagtggtattcctgttctttgtctgacataactgtccacacctaaaagctgggcacagactttcccatctggtgggaaatatttcagaaacattagtgacacttgcagtgtcctgctattgctgcaaaacttgctcttccttcagcccttgccatggaattcaggtataattgtggtcttctattggcacatcctttatttacagacatacctcagagatattacgggttcagttccacaccaccagaaaagcaattatcacaaagtgaatcacatgatttttttgacttcctagtctataagagacttattttttcactttttaatcatagccattctgactggtgtgtgagatggtatctcattgtggtttttgtttctgtgttttgttgttgttgtttttgttgttgttgttcttgttgttgttgtttttgagacggagtcttgctctgtcacccaggctggagtgcagcggcgcgatcttggctcactgcaagctccgccttccgggttcacgccattctcctgcctcagcctcccgagtagctgggattacaggcgcccctcaccaggcccagctaatttctatatttttatagagatgggttttcaccatgctggccagggtggtctcgaactcctggcttcaagtgatccacccccctcggccttccaaagtgctgggataacaggcgtgagccaccacacccagcctcattgtggttttgatttgcattctctaatgatcagtgatgttgagttttttcttcatacgtttgtcccatgtatgtcttcttgacatgtatgtcacttgaaaagtgtctgctcacgtcctttgcccacattttaatggtggttttgttgttgttgttgttgttgttgtttttgttttttgttttttgcctgtaaatttgtttaaattactgatagattctggatagtagaactttgtcagatgcatagtttgcaaatattttctcccattctgtaggttgtcagtttactctgttgaatttcttttgctatgcagaaattcttaaatttaattacatcctatttgtcaatttttagttttgttgcaatcgctttggcatttttgtcatgaaacttttgccagatcctgtgtctggaatggtatttatgagcttaccttctagggtttttatagctttaggttttaagtttaagtatttaatccatcttgagtttattttttgtatggtataaggaagtggtccagtttcaatcttctgcatatggctagccagttatcccagcattgtttattaaacaggaagtcctttgtccgttgcttgctggagagattgcagagaaaagggattgcttatacactgttggtgggattgtaaattagttcagcaattatggaaaacagtgtggtgattcctcaaagaacttaaagaagaataaccattcaacccagcaaccccataattgggtatgtgcccaaagaaatataaatggttctaccataaagacacatgcacacctatgttcactgcagcactgttcacaatagcaaagacatggcatcaacctaaatactcatcaacagtggactggataaagaagatgtgatacatatataccatggaatactatgcagccataaaagattataagattatgtcctctgcagggacatggatggagctgggggctattatagtaggcaaactaactgaggaacataaaacccaataccacatgttctaacttacaagtgggagctaaacagtgagaactcatggacacaaagaggggaacaataggcactgggacctacttgagggaggtaggtgagaggaggaagaggatcacagaaaagtgcttattgggtactaagcttattgcctggatggcaaaataatctatatccctgttacatgcagtttacctatataacaaaccttcacatgtacctctgaacctaaaataaaatttaaaaaaagacctatgtttacactatactatagtttattaagtatgtaatagaattacatctgaaaaagcaatgtacataccttaatttaaaaatattttcttgcttacaaatgcaaataatcatctgagtcttcagcaagtcataatctttatgtgggtggaggatcttgtctggatgtcaatggctgctgactgatcaaggtggtggttgctaaaggttgagtggctgtggcaacttcttaaaataagactacaatgaagtttgttgcattgattgactcatattttcatgaaagatttccctttggcatgcaatcctgtttgatagcattttacccacagtggaactcctttcaaattggagtcaatcttctcaaaacctgccactgctttatcaacaagtttatgtaatattctaaatcctttgttatcatttcacaatattcatagcatcttcactgggagtagattccatctcaagaaaccacttactttgatcattcatgagaagcaatccctcattcattcaagttttatcataagattacagcaattgagtcacattttcaggcttcacttctattctagttctcttgctatttctaccgcatctgcagttacttcctccaccgaattcttgaacccctcaaaatcaaccatgacgactggaatcaacttcttccaaattcctgttaatgttgatattttgacctcctcctatgaatcatttatgttcttaatgacattcagaatggtgaattctttccaaaagttttccaacttactttgcccagacccattcacggaatcattatgtatggcacctgtagccctaaaaatggatttttttttttttttttttttttttgagacggagtcttgatctgtcactcaggctggagtgcagtggcatgatttcggctcagctcgctgcaggctccatcttctgggttcccgccattctcccacctcaacctcccgagtagctgggactacaggcgcccgccaccatgcccggctaattttttgtatttttagtagagacaaggtttcaccgtgttagccgggatggtctctatctcctgacctcatgatccgcctgcctcggcctcccaaagtgctaggattataggcgtgagccactgcgcccggccaaaatggatttcttaataagacttgcagatcaaaatgactccttgatccatggagtgcgggcatgaaaacgttaatctccttgcagatctccatcagagctcttgagtgactaggtgcattgtcaatgagcagtaatattttgaaaagaatagtttttttctgagcagtaaatcttaactgtgagcttaaaatattcagtaaaccatgctgtaaacagatgtgctgtcattcagactttattgtttcattcacagagcataggctgaatacatttagcataattcttaagggccctaggatttttggaatggtaaatgagcactggcttcaactgaaagtcaccagctgcatacagttatccctcagtatgcatggaggattcgttccaggactcagatgaataccaaactctgtgcagtccctgatataaaatggtgtggtggccaggcatggtggctcatgcctgtaatctcagcactttgggaggctgaggcgggtgcatcgcctgaggttaggagttcgagaccagtctggccgacatgaagaaacccccctctctactaaaaatacaaaaaaatgagttgggggtggtggcaggcacttgtaatcccagctactcgggagtctgaggcaggagaatcacttgaacccaggaggtggaggttgcagtgagctgagattgtgccactgcactccagcctgggtgacaagagtgaaactccgtctcaaaaaaaaaaaaaaaaaaaggtatgtgtggtatttgaatataacctgggcacatcctcccatatactttcaaacatctctacattacttatgacacctaatacaatgtaaatgctatgtaaacagttgtcatattgtaatgtttagggaatagtgacaagaaaaagttggctgtacatgttgggtacagatgcaatttttaaaaaatatttttgatccatggttggttaaactcacagatgcagaatccacaaatatagagagccaactatacttctaaattccataattctcaattttatcttttgtagaagaactggactagattatctctaaagtgtcttccacttctatatttcttccagtccacattaataggatcattgttaggctggtatctgtttctaaagtactacagagtaaatcaacaaggaaagcaaggttgtcttctcattcagaaggccagaggttgctaaatactccagttctgaattgtaagtgaaacagggaagttatatactaggtgattggtgtgctgttaaatgttttaaccttggctttgctctattaactttatctgtcctttttaaatcactttcagaaataaaataaggtgaatcacctcaagtggggtacaccaatgagttgagatcaatttgggacgtccttcatattgacctttgttgatacaaattatctttgtcacagaatattgctgtgattccattacctcctgttgtacttgcctgagcttcccattgttttagtcactctgcatcactgtgtacgatgtcaattgtgttttagatggttaaaaaaaaggaaaagaaacatttcagtcataccaaagtgttgatttgcaataaagagtataatttactgtggctctgggtattgattaatctatacaatgaaaatggcttttgagtgctttggttatcagttgaaattatctctattttttaagctgataattactgaaaaatgtcacaagtggaaaaaagggtgcaatacaaactagtgttttatccagatatctattctgcctgggaaaggtaaaaggatacacattatggttgcaatacggtctggatgtttcacttctgtgtatgtgtatttcttgcccatctcaaataataggtgggactttttttcacttttattttaagtgaaggggcacatgtgcagaatgtgcaggtttgttacacaggtaaacatgtgtgatgagggtttgttttacccattatttcttttttaaaaaaatttcaataggtttttggggaactggtggtgtttggttacatggatgagttctttagtggtgatttctgagattctggtgcaccctgacccaagcagtgtacactgtacccaatgtgtagtctcttatctctcaccccctcccaccatttcccctgagtcctcaaagtccattgtatcattcttatgcctttgcatcctcatagcttagctcccatttgtgagtgagaacttacgatgtttggttttccattcctgagttacttcacttagaataatggtctccaattccatcaaaattgctgagaaagccattatttcattcctttttaagacaaagcagtattcatatatatttgtatatgtataattatatatgtatatacacatagaataatatatatattaatcaacaagtgattaatgggcgtttgggctggtgccatatttttgcaattgtgaattgtgctgctataaacatgcgtgtgcaattatctttttcgtataatgacttcctttcctctgggtagatactcagtagtggaattgctggatcaaatggtaatatctacttttcgttcttaagaactctccacattgttttcagtaatggttgtactagtttatattcccaccaacagtgtaaaagtgttccctttttaccacatccatgccaatatctattattttttgattgtatcaattgttgcaggagtaaggtgatatctcattgtggttttgatttgcatttccctgataattagtgatgttgagcattttttcatatgtttgttgcccatttatatatcttcttttgagaattgtctattcacgtccttagcccacttttgatgagattgtttgtttttttctggctgatttgtttgagttcattgtagactctggatattagtcttttgtcagatgtatagattgtgaatattttatcccactctgtgggttgtctgtttactctgccaagtatttattttgctggtcagaagctttttagtttaataagtctcatctatttattgttgtttttgttgcacttgcttttgggttttgaagtctttgcccaagccaatgtctagaagggtttttctgatgttctagaagttttatggcttcaggtcttaatttaagttcttgatccctcttgaattgagttttgtataaggtgaaatatgaggatccagtttcattctcctacatgtggtttgccaattatcccagcaccatttattgaatagggagtcttttccctactttatttttttgtttgctttgtcaaagctgtaaacattgggctttatttctgaatcctgtattctgtcccattggtctatatgctgacttttatgccagtacctgctgttttggtgactatagccctatagtttgaattcaggtaatgtgatgcctccaaatttgttctttctgtttagtcttgctctgactatgcgggctctttttattttcatatgaattttaagattattttttctagttctatgaagaataatggtggtatttttatgggaatttgttgtgtttccatttgtttgtgttgtctatgatttctttcagcagtgtttgtagttttccttgtagaagtctttaatctccttcgttaagtatattcctaagtattctattttttttttttgcagctattgtaaaaggggttgagttcttgatttgattctcagcttggccactgttggtagtatagcagagctactgagttgggtacattaactttgtatcaaaactttgctgaattcgtttaccagttgcaggagctttttgaataagtgtttagtgtttcccaggtatacaattatgtcctcagcaaacagtgacagtttgacctcctctttaccaatttagatgtcctttattcctttctcttgtctgattcatctggctaaaacttccaatactatgttgaatagaagtggtgaaagtgggcaaaatcctgcactaaccaaatcccacagcatatcaaaaagataatccactatgatcaagtgggttttgtaccagggatgcagggatggttaaacatccacaagtcaataaatgtgatacactgcataaacagaattttaaaaaatcacatgatcatctaaatagatccagaagaaacatttgacaaaatccaacatcactttatgattataaccctcaacaaaatcagcatagaagggacataccttaaggtaataaaaagccatctatgacagacccacagacgacatcatagtgaatggagaaaagttgaaagcattccccctgagaactgttgtacagatgatttcatcacctaggtattaaacctagtatccattcgttatttttcctgaccctctcctcctcccaccttgtcacacaccctctgataggccccagtgtgtgttggtttcctttatatgttcatgtgttctcatcatttagctcctacttatgcctgcaaacatgtcatatttggttttctgttcctgtgttactttgctgaggataatggcctcgtgctccaaccatgtccttgaagaggacatgatctcattcttttttttgtggctgcataatattctgtgttgtatatgtaccacattttatttatccactctatcactgatgggcatttaggttgattccatgtctttgccattgtgagtagtgcagtaatgaacatatgcatgcatgtgtctttataatagcgtgatttatattcctttgggtatatacctggtaaagggattgctggatcaaatggcatttcagtctttagttgtttgaggaatcaccacaatgtcttccacaatgattgaactaagttacgctcccaccaacagtgtataagtaagtgttcctttttctccacaatctctgcagtatctgttatttttttaccttttaataatagctattctgactggtgtgagatggtatctctttgtggttttgatttgcatttctctaatgataagtgatgtagagtttttttatatgctttgttggccacaacatatatattgttggccacatgcatttcttcttttgagaagtgtctgttcatgtccattggccactttttaatggggttgtttgtttgtttttttttcttgtaaatttgtttaagttccttatagatgctggatattagacctttgtcagatgcatagttggcaagtattttctcctattctctaggttatgtgtttactctgttgatctatatatatatatatatatatatatatatatatatatatatatattgctgtgcagaagctctttagtttaattagatcataatcttttatcgtatcccttaactcaaaaaaatattgaacattaaaatattggtagcttttttcccactatcaacctccatagtaaaactatagttaggctttccttcaaatgaaaatccattcattttttatactgtttgcctgaaacaacacagtattctttcttaatagtattttcatgttgagctaaacaaacaacatttaaataaggaaaaaatattattatttcttttttattatactttttttccttcttctttctttctttcttcttttttttctcttaatatatggggtctaactctatcacccaggctggagtctagtggtgcaatcatagctcattgcagtcttgaaatcctgagctcaagtgatcctccctgctcagcctctgagtagctgggaccataggtatgtgccaccatgcctggctctttattacatttctgttataatattttctctattatatttcattttagttactaaagaatatgattttgaacttttttggaaagatcatcattttactctttgttgtagaagaaaatctatgatggtctaatgaactacggaaatattaacataattttaagaataatacatatatttgtattaccaaaggctatgcttattagtcattcaaacttcaactaattttttctttcatgaaatactcagaagtatctattcaccaccaccaacactccctccatcatttaatttcaagcagacttgggtttattcctggaaatgattttttactagtgttcttaattttagtcttcttctttctccaatatcttaaggcagaataatttttcttaaatgcaactttcattctttctccataaatttgaggtatcttcacatgaaattgagcttcaaagaagatgaagatgagtaagggccgggaaagccagggaaggtttctgtttcctgtgtatcaagtttcccaaacaagaatcttggagtcctcttcagcttctgcaactccttctcgatcaacatgcagtcaaacttcaaattgtattgacggaaactcagaactgcttagatcattttcttactctcattatttctgcgtggatttagacattcattgtcttttgcctgggctcttgcaataggcttttatcttatatcctaccgtaaaaggtttcagatgctacacatcctccatactaccaaagtaataggtaaaaaataaatctgattgtgtctgtacaaaaaaacctgttagaactaataaataaattcagtaaaattgcaggatacaaagtcaacatacaaaaatcagtagtgtttctaaacactaagaatcaactatttgcaagagaagtcaagacaacaatatcattttaaagcatcaaaaatattgaggaataaacttaatgaggtgaaatatctgtacaccaaaaactataaaacatggattaaagaaattaaataagacacacataaaaagaaagatatcccgtgttaacgagtggaaagaattaatgttcttaaaatacccagagtacttaaagtgatctgtggattcaatgaaatccgtataaaaattccaatgacataatagatattttttaaatcttaaaattcatatggaatcacaagttaccccaaacagtcaaagcaatcttaagtaaaaataatgaaacttggttgcatcacactgcctgatctcaaaatacattacaaagttgtagtgatcaaaacagcttggtactggcataaaaatcagcacacaaatggaacagaaccaaaagcccagaaataaatccatgcacttatggtcaataaatctttgacaaaagtaccaagaacacaaaatgggaaacgaacagtctcttcagtaaatgatgttggaaaaacttggtatccacatgcagaagaatgaaattggacccttatctcacactatatacaaaaatcaactcaaaatggattaacaacttacagaacacctaaaactgtaaaagtactagaagaaaatataggggaaaactctatgacattgatctgggcaatgattttttgaatctgaccccaaaatcacagaccaaaacaaaagaaataatcaacagaatgaaaagacaacctacagaatggaagaaaatatttgcaaaccatatgttcaataaggggttaatgtctaaaatatgtaagaaactccaacaactcaatagcaaataataataataataataacctgattacaaaatgggcaaaggacctaaatggacatttctcaaaagaaggcatacaaatggccaacaagtgtatgataaaattcccaatatcactaatcatcaggaaaacacaaattaaaactacagtgaaatagcacctcacattcattaaaaatggctactatcaaaagaacaaaagataagcgttggtaaggatgtggagaaaacaaaagtgttgtacactgttggtgggattgtaaattagccatcatagaaaacaatatgtaaattcctcaaaaaagtaaatacagaactattgtatgatccagtaattctacttctgggtatatacctagacattcaaagaagaattggtaccaaccctactgcaactatttcacaagatagagaaagaaggaatccaccctaaatcattctattaagtcagtatcacccaaataccaaaatcaggaaaggacacaagaaaaaagaaaactgcagactaatgttcctgatgaacatagatgcaaaaattctcaacaaaattctagctaagcaaatccaagggcatataaaaaagataatacatcatgatcaagtgggtttcatacccgggatgcagggatggtttaacatatgtaactcaataaatgggatacatcacataaacaattaaaaataaaaatcatatgatcgtcttaatagacacagaaaaaatgtttgataaaatacagtatccctttatggtaaaaaatcctcaacaaaattggcataggagaaacatacctcaaagtaataaaagccatctgtgacaaacccagaaccaatatcatactgaatggggaaaagttgaaagcattccccctgagaactggaacaagacaaggtgacaaaattagtgtgataaagagatatttacactgtcgtgtttattgcagcattatttccaatagacaggatacggaatcaacctaagtcttatctgtagatgaggaacggataaagaattgcacacacacacacacgcacacacacacacacaatgaaatactcttcagccttaacaaagaaggaaattctgtcacttgtgacaacatgaatgaacctggaggacattaggttgagtaaaataagccaagcacagaatgacaaatattgcatgatgtaacttaggtatggaatctaaaaatgtcaaactcacagaaacagaaagtagaatggtggttaccaggggctactgggtagggctgtaggggtggaggaatggaaaatgttggtcaaagggtacaaaagttcagttaaacaggaggaataagttctggatatctattgtactatgtaatgactgtagttaataatactgtgttgcatattcaaaaattgctatgtgtcgattttaaatgttctcaccaccaaaatataaatatctgaggtgaaggagatattaattagtttgatttaatcatttcacaatgcaaatattaagttgcacactgtaaatatatacaatttttaattgtcaattaaatataattgaaaaatagaacataataaaatattggattattaactctatcaggaaatgaagctttgagtaacattttactgtttatatttatggttttagccaccaagcaaacatggtggtcaagaacttggactcacattcaggttgccatagtccaaattcaggcttccctactcactaatgtgttcttaagcaagaaataaacttattttggcttcctttggtaaaatgggattaataattgttcctatcctacattttattgtgaagattgaattaatgaaatatacacgtaaaacacttaagacaattcctggccttagtaagatttcaacaaaaatggtcattattatgatgctttattctgttacatttatggattattagacagataaaattggacctcatatgtgaccattctattttcctttattcaaatggaaaaagaatgtcaattcaacatagggaaatactatatatgtataaattattacttaaatttatgtttttacttaaatatatgtttattaaaatcaagtgtttaaatttatatattaacttttacatttataacatcttatttcttctgaaaacaaccaacacaacccatttcttatcagtgataattcaaatggcctctttattctttcctgaatttttcaatcccaattcttctcttctgttctttttcagcagcatcttgcacagggctttatggaaaagactgaggtgctgtggtgaatttaatctcctctgttgtttactttaatctcttcatccagttactctgacttttctccatgcttaatgttatccctcttgatttcacagcttaacaatttcttctttgttcttgaccccatcctagccttcttatttcaaaagtcccctcaagcaagtttcttttcacattcttaatttcttattcttaataactccttttttatgccttcaagcaagtatggctctctctcatactgaaaatgtgctcttccgttctattttgctactgtttttattcacttaacagtaaatttcaaacaagttttgtgcacctaattctttcatttctttacaacccacccattgcttatccactcttacagctgtactagaattgtaatttaaatatgtatcatatcagtttttttgtaacattgataatttataatatttaacatattttgtaatagagttttcttcttaatattttactttccttttaaaaagcctcagcaagttgccattgactttcggcattgtatttgatacctctggactctggctactatcgccctttctgtataaaatctgtaacttgaccaaggcaagaacctccaccccaggccaactaattttctcatctgctattgtcccaacctgctttggtttctcctataggtcagagtcaggtcctgcttgggtttgggcctttgctcataattttcccttgcaattgaaatattctctcactccaggccagcctttaaagcctaacgtaagtaccaccacatccacgaagcctctgcacatcaacttcagatctcaatgctgttaaccttttataaacaaaaagcaatatagaataaatcctttttgggagcaggagtcaaaatgctagttgcagtcttgcatctgtcacttatttgcttggccagtgacgtagccttattaaaaactcaatttctttatccaaaagatgaggatgtttatacctccctgatctaccttctaagattgttgtacatgataagtaaaatactgtgcataaaaacatttgtaaaaattttaaaaatctgttcaagcttgtcattgttaccacagataaatcgggtactaccttgctgtcttggattgcttttgtatgttgttctcttatgttgttactggactcggtgttattacttaaatttctctgtatttattttgacaacttaattaggttttgaattctttggaagcggagagtatgaatggtattatgagcaaagacccagaaggagccacctcctaattattttggagagaattatctaagaggcctaattggacttgtgcttcccattaaagtatgattcatatcagagaagcttacagtcttattgctggccacatggagatagtacataggagatactcgatgggaattctgtcaactgaaagagagtcagcaagacagtgtaaacatggttagcgtgtctcagcttacccagcctggcttggtgtaaccaatatagactatggaaaataggaagtagacaaaaggtagtggagattgggctcatgccattttctttaattgaaatacaatttccataaaataaaatacagatatttttaagtgcatattactttgagttttgacaaatgtattcccctgtctggcaactgctccaattaagatgtagaatatttctatcaccccagtacccttttcctgtcaatccctccttctcccagagacaatcactgttccaatttctatcactatagcttagttttaccagttcaaaaacttcacataagtagaatcataaagcatgttactttttttcctggcttttgtcacttaacagtttttttttttagatttaaccattttgttgcatatgtaagtatatcataactttttgttgttgagtagcactctcctatataagtatgttataataatgtttcatttacctattgatgagcatttgagttgttcctaagttttaactagtataaaaaagtttactatgaacattcttgcataggtctttttgtgggtgtatattttcactactcttaagattgtcagactggataaagagcaaagttcagtggtatgctgttttcaacacatattttaaatataacacagacaggttgaaagtaaaaaaatcagtaaaagatacagcatgaaaacactaagcaaaagaaagctgatatggctatatgaatatcaaaaaagtagattttaagacaaaggatactaacacagataaaaatggtcattttattggggaaaaaaaggccaatttgtaacaaggcatgacaatcttaaaagggtataacaaagtagaatttcaaaatccaagaagaaaaaaatggcagaataaagtaaaaagcagagaaatccacaataattgttagagattttatcaattttatcccagtagttgctagaacaagaagacaaaaaatcaatgatatagacaacttgaataacattatcagggatcaaacctgcagacctaactgacatttatagaatattatacccaacaacttcagaatatacattcttttcaaatacacatggaacattccacaggtggtgttatgctaggccataaaattagtgacacttcatttcaaagagtatttgactacaatggaattaaattagaaatcaattacaattagatatatagaaaaggcccctttttacaaaattaagcaacagacttctaaataatcaatccatggatcaaagagggaaatcacacacacacacacacacacacacacacacacacacacacaaatacagtgcgctatacggagtgataattagaatacaacatgttaggaatctctgtgttcatgcctttaaccatttcatattatttcataataatcaagaacttattaatcaaattagcatttaattatcttcagatgaaatgagaaagagggtgggcaaaatgtagactgaaggctgagaattataggaattatggtcacaatcagccagtgttttaactctactgcaattgtaacaaagtcattggatcttggttcaaccaatgggaatagatcctttcaaaatgccaataaataaaatgctaaataaacattttggcaattttctcttgataaatctcttatagcaatgtgcataatgcttccattttcatccaatgaagtactagtgaaaatagtctagatagtgttaatattgatagaaattataactaaggaataagttagatggtgattaaagagttgaataagcactcaaagaaagttgtgaaatctctgttctgagatatgtgacaaaaccatctgtctctgattttagtttaatccttaatgaatataagaagcctgactaaatgatctcttgggaggcccccttccaaacacaaaacccattattttaatttgcattggttatagtgccaacacaaagaaaaaacacactcaagatcttttttataaagtagggatttaattctaaatcttagtcatatcttacaatttataaattctttaatgttgaaaaatattttctttgtagaactaaaaccattctctctctctccctccctccctctctctctttctctctgtctgtttgagaacccaaaataaaatgtagtttataatttgtgattatgatttgaacttttcaaaaaatgtatgagtcatttcttgccctttgataatttccaaatgatgatgctttgcttttgcttttagagatcagtctttgcttttaattatttccatatattttggttgggagtgagaaaaataaacggaaaatgtcactcatgagtgtcagcctggtaatgaatatatagaatgcaaatggtcactttactgggggagacggtaagtttttctgagccttttgcccttttatcttctccagcctgctgttttttctgttttcatttgtagattaaactggtgcagcaatatggtggggtagaaatcacatgaatctgacatatgtgggctcctgtatctgtgcacatggatagagatgctggtcaaaatccaaccatcttatgagttccagacatacttttaaaattcacatgcatgaagctatacaaaatattttaaaatacagaatccaaaataaaatttctttttgtataaaatactccttctcaattcattcctgtaggcaaaacttgatatttaataatattaagaaaaagaagaaaatgaattcgaaatattttattgctttacaacacaaaaaaaatggtttacttactcccaataatctgaatttattccaatagtatatttatggatttaaccaggagagggattatccttattctaagtaggggagaaaaaattaaaaaaaaaaaaaaagaaaggaaggaaaaactccagtggtcaagagattgagtgaaccgaggaacagagtggttatttgttctctgtaatgtattaggaatgactggaagtaatggaatatgctagtgaatatagctacccctatattgaatagaaaaatacattaccaaaacacttttcacacacgaaaacaaaagaacattttgtacaaatacctgatgtgtagctatatcatttagttgctactattgcctagtttaaaaattgtaccctggttaaaaacagaatcccttttctaatcttttaagatttcaacaaatatttccaggcattaaggacaggcagtattttgccttgtccacttctctgacgtctagctgtttctagttgctaaatttggctgctgtacttttcatatccgtaaagctgttcttgggtccaccctgcaggcagccatccaggtttggatttttccgggcagcccgggcttggctatcctgctttcatcatcctccttctgttctcaagccaaggacatagaatggcagagagctcccctttatttctggtatgattgttgcttctccgggtctggcttgggctctgatcttgacatttatgtcacaggtgtcactggtgaaactgctgtgaaaccagatggcaggcctaactgaggttggcagcaactgctgttgtccccagtgtcactgctacctgctctactttcttattcctgttgatgttctactgccttgttttattgcattcgttcaatgttaccttccaacaggccttttcaaatgtgaacaccattccataactcttctgatgcattaattccccatggaacccattcatctagggaggctcatgacttggctagattctccactttccttcagggcttttgttaaacctcatttcttcagcaagcctctccatgaccaccctatctaaaattgcaatacatccttctcctccccaaccctgattattttttctctctttttcataaattttctttccctggagcaatttgtaccacatgtcagaatctatatttgccttacttgggcattatctgtatccctccactgggttacctagaatataagctccacaagggcaaggttttgtctgtttggttcattactgtatcccaagcatctggaagaagactggcccttagtagacatttcaaaatttttgttaaatgattagctggattgtaaatactgaaagattaggtttattgtttttgatttggtttcattcattgtgcttagtacagtgctatatgtaagagcttgaaagacagagaaaagagcattagtagtatctgttatgtatactggatgttttacttgagttatacccttttttttttccaagacagagtcttgctctgttgcccaggctggagtgcagtggtgtgatctcggctcactgcaacctccgcctcctgggttgaagcaattctcctgcgtcagcctcccaagtagctgggattacaggcacccgccaccacgcctggctaatttttatatttttagtagagacaggttttcaccatgttggccaggctggtcttgaactcctaacctcataatctgccctcctcggcctcccaaagtgctgggatggcaggcatgagccactgcactcagccaagttatactctttaatcctcacaataatctaggtattttgaccaaagaaccacagctagaaagtaggagagtaagatgtaaaccagttttgtctggctctaaaattactgcctttttttgtttgtttgttttgctcagaagaattggtcatgccaaatagtaataagagttcttcaaaatgaatgatatttcataataaggtgtagctagaaacagtttgccaaaaatacttaggaaaaaaaaaagatttgtgaattagctccctaaaaatacaaacgcaacaattccaaaagaaggaattccaaagttattgggccatagtacttttgtgttaagtgcctggccctccaaggtaactaattttaatttaaaaaaaatgtctggacacatattttgacatgttttgctgaaaatatatttataatatgagcccaacaaagacaaatagattcattcttggtgctaaaaaatgacaagaaaacttctacgccattgctctcgaatgctttcttcattgtagtccaagaaaatttgattagttgtattttaaaagtaagccagatttcactggtaaggtctttccttacttcatcttctcccttgacaccttttgatttggttgtacaaatgcaggttaggccccattattaattcagtaccacatatttatgaagaccccacatgatgcaatcagcactagagactggaagtttttcatcaagcccagtaagcccacagtaaaaaggaatcagtctcaatcactcatcaggaagctaacatgttctgtgaccaccttctggatgttgagttatctctaatccagagtttatgtcaatgatcttaaaaattagttcaattctggatgggcgcggtggctcatgcctataatcccagtactttggaaggctgaggtgggcggatcacgaggtcaggagatggagaccatcctggctaacatggtgaaaccccatctctactaaaaatacaaaaaattagacaggtgtcgacagagcaagactccgtctcaaaaaaaaaattagtttaattttgtcttcattttttcttacttagtaaaagataatgtaatgcctggtgtttttttaaggcaatcatagaatctggaggttgaaaatatttggggaattatacattctaattattcagacaggcatccttactgcagtatctgctagtaattgaggattatttcatatttaaaaatactcatatttttatgcaaatctcagaatttgaacattgttacttataattgttaaaaagtaaattgagaaacaataaaatgttaaagagcttatttgagcaaatggtaattcttgaatcagctccaaaccaaaagtggttctgaggccacttgagagaactcaaagagaaagcttttatagggagaatgtagaaataaagcaaaggaaatatttaattggttgcaattatatagttgccttatttggtctatcttgctgaaaagttcctacttgtattactacatgagttggcagcttctgattggttagccttaagtttcatttctctttaggcatttataagaaatagttcaagttgagttttgcatatgtttgcaaatcaagcaaagtttaggttacttctgagggccacttggatttgtctgctcagaacttcttcaagcctggtctgttttaatttacttcaacatattttatcaacattttgctttctataacattcatagtaaccttagatctgccctctagaatcgacagaggctatttcacccccagcctcttttcaacatgagagtttgctcaatgctgcttctatttgaaatcttctgttattcagctcttttaatgaatatctactttctgcaaggcattgctttatgtactgcaagagttacaaagaggagtacatcataatcttacttgaaggcaattacttaaatagaaaaaatggtgtgtgaaaaacaaatgttcttaagcttgaaaaagtgaaagtgtgtacccagctggaaggatgtgaaagtgtgcatggaaaatgtatttgagatttaaactgctgttgtcttctgggttgaattggtgtcatgaacttcaggtagtgaaaatagcttaaacaatacccctgaagtggaaacacagagattagtgtgaaaatttagaagaccgtgagcacattatattttgtgtgtaccataagacactggtagagtatattagtccattttcttgctgccataaagaactgcctgagactggaaaatttacaaaggaaagaggtttaattgattctaagtttggcatgactggggaggcgtcaggaaacttactatcatggcaaaaggcaaagaggaggcaaggcacctttttctccaggcggcaggaagaagtgctgagcaaagaggggagagccccttataaaatcatcagattttgtaagaactcactcattatcacaagaacagcactggggaaaccacccccatggttcaattacctccacctagtctctcccttgacactcggggattatggagattaccatttaagatgagatttgggtggggacacaaagcctaatcctatcgtgggggttggtaagagggaagactagaaaggaaagctggactaggcaaagtggctcatggccataatcccagcagtttgtgaggccaaggcagggggattacttgagccaggagaccagcgctggcaacatagtaaaaccccatctctacaaaatgttagaaaattagacaagtatggtggtgtgcactatagtcccagctacttaggaggctgagatgggaggatctcttgggcctgggaggtcaaggttgcagtgagtctttatcacaacactgcactccagcataggcaacagagcagaaccttgtgaaaaagagagaacgagaaaagaaaaagaaagaagtcaagaaagaaaggcaggcaggcaggaaggaaggaaggaatgaaggaaggaagcagggaaggaagcattctttacatgccaggaagaggaatcttagtgatgagaaaactgagtctctgagagtttgagcctcactcaaggtcactgagttgctaaatggcagaactggatttcaatcccaataatttgattcaaaacccatactctttcccatataccacattatctaggaggtgtcccaagagttctgaagggggagttagcttccatctcaggagacgaagagagtcttcttagaccaaatggtatttgagataattttgataagtttatgttgaattcattggttgagatagggaagacaattcatttcaaatggaagaaactgagcaaaggtacaaaggggaggaagtggagagattgggaacagaaaataattaatctttggttgaacatcatcaaaaaggtataaaggatggaaatgaaagctaagatcagagcacaaaaggataacatatttgaacttttacttggacttaaatgataaagtttttagacagattattattataactacagccaagtttttaaaaatattatattagtaattatatataaaatagatggatgggaaacaaacggaagaatggagattatctgtttagagtaacccagttaattataataaaatattgaatctgcatgttggcaatggaatgagaagggaagagatagatatgtaatttcagaagcacaattcacagaacttgatagagattagtggaaggaggagggagatagatgacattaattctgtgatatggtttgcctgtgtcccacccaaatctcattttgaattcccacgtgttgtgagagggacccagtgggaggtaactgaatcatgtgggcaggtgtctcccgtgctgttcttgtgatagtgaataagtctcataagatccgatggttgtataagggggagtttccgtgcacgagccctttttctttgcctgccaccattcacgaaatatgtgacttgctccttcttgccttctgccatgattgtgaggcctccccaaccaacaggtggaactgtaagtccactaaacctctttcttttttaaattgcccagtctcaggtatgtctttatcagcagcgtgaaaacagactaatacattctgtaaatttgaatgcaggagatagaacctttaaaagcaatggtaatttgtcttgcaaggcctatgtttatctctactatgtactcagactattgtacagcctggaccatagaaggtatgcaatacgattatttatctatgtactcaatccctactctgtgccagggattgtcctaagcactagcgatagagtaaaaagaaaagaaaaaactcttgttctcattagtaaaataaacatcacgttgcttcacccaaactagatcaccttagtcccttttattgttatctctgtgtcactctcctggcaggtgagtgtttctgcttttaagaatctgtacctgtgtcttgctttagataactgtcttcagattatctgagtcattttttccacttcagcaccaagtctgaagtgccggtgagtttacgtgcccccacccatatccttagcgaagactgactattgcagtcttgtgaaagtacagatcccttgcttcatatagggacaaactcaaatacataatttgtactccaaagttgtcctgtgggatcataataaagccacttctgtagggctttgtttggaattacaccttagcttggctccttccccatccagacctgcagctttacccactcctttcctgattgatcctaggagtacctccttaactaatcactttcccattaagccacatctcagtgtctgtctctggggaacctgacctaagacatgtagtttacattgcaatgtacagagacaaataataaacaatataaataattattaataagtttcatactaggccgggcatgttggctcatgcctttaatcccagccagttgggaggccaaggcaggagaattgcttaaagccaggagttcaagaccagcctggccaacatagcaagaccctcatctctacaaataaaataaattagccaggcaaggtggcatgtgcctctagttctggctacttggaatgctgaggtgggaggatcactggagcccaggaggtcgaggctgcagtgagctttgccactacacactagtctgggcaacagagcaagaccctatctctaaaaaaatagatattttattaggggaagttgaagttatagagggaaataaggaaggagatgtggttagagtgtgtgaaagtgtgtgtgtgtgttttcagttgtaggggatggtgttcaattttatatagcatggttggggaaggcatcactgggaaggtcctatttgagcaaagactagtaggtgctaagggaatgagccatgcaaatttctgggaggaaagcatttcaagcagagatgaaagcaagtgcaaagccatgagctgggagtgtctggagtgcttggtgagaagtagccatgtgagaaagtacagggcaggagacgacatgagagaaggaagtgaggggggtgggcagaccttgcaggccaacacaagccatggctcatactttgacttttcctctgagtaaatagagagtcactgtagagttctgagcagaatagtgactcgactgtttttgcgtttcaaaggaatctggctaccacattgagaataaactgaaggaggccaaaaattaagcatggagaccaatttagacattattaccagggctggcttcgtgggcagccacacaaggctccatgctaagaagggccccacacatggagcctaatgctctgcagtcaccatcttgataattttaataatttgtgtttgaatttaggttttgtaagtgaaagtcatgggactgtggagcatgttctgggtgctcagtgtctggtacttcctgctgtggataggttcaacactacctgcacccctgctccctgcccaggccactcagtgccccctttttcacctttttccagcaactattgccaccctccaacagggagaatgctgggctgctggtgggaagatgtctgtgttctgctgcccttaagcctgtcggggatttgatcacaagctcaggtcaagttgcagttaggaatgcacctcaggctggaagtgccaaggcaggtgagagactcagtgggagagagcttctcacccacctctaatccaggcatgtcaggtgcagagttccaatgctttggggggttgaccttccaccagtttgggtggcaggctcatgggaagggtagattgacttctctgaccccaattgagggcaggttgcttcagtccagcaactggtgggaaggggaatttggcagtgggggatgggggcagacattatcttctgctctgaatcagggacactcatatggctctgtgtttgccctgcaagtgtttccctgctccaatgagtacaacattaaatagcaaataaaagccatcagatcagtagacagtttgcataaataagaaaaaagctatgttgcataatcttttaatggcccctttttcctgctacttaaacaagtgtcttcctcttttttttagagacaggtcttgctatgttgctcaggccagtctccaactcctagcctcaaggaatcttccagtttgagcctctggagtagctaggattataggtgcaaggcactactcgcagctggggcctcaacattatcagttttgcactgtacttcataaattactggccctagcaattgcagtaatctaagtgaatgatggtggcaacttgaaccagtgtgctaatggtagaggaggtgagaaatgattggatccaagatatattttgaatatagaacaaaaaggtcactagaggttatgacatagtgtatgagagagagaggaaggaatcaaggatggctgtaaggtttttcacctgaggaacttgacattatttgttgaatacataaatgatgtgtcctgttatgtatctgttcatgaaacacattgatggtaatgaagatgcagtggcagaagcaaatcatagaagtatttcctagcggtagaaatattaagacttggtgacaaagatcataaattaaaggaaagggagtgaaacaactaagtcaggaaaaatacttctaacttaggaaaaaagaagaaaaaggaaggaaatggaaatatttaacaaatggtataaatttctaaggaagataagttcaattttggaaatgttcacctgaggctccaagaacctacagcccaggagaccttagctataggttgttgggaatatgtgataggatcctgaatgagcactcaggactagggatctatgttgagggcatcatataagtgtaagctggagtcacaagaatggttaactcatcaataaatgaatatgaggagagatgtatacagtattgcagaagagagttccttgtaaatgtgtaagctcatcctccatgcctgggataagagtacattcattatggtgcatgtgaaggtattgagaaaacaacttccgctttctggcagaggttaagctagtcccataatagcaatagcaagattataaggggaaactatgttatttgtactgggtcacaaatttgtctaatagcagtggacctgtgggattctataaacatcttaggtatagaaagggatccagtgacttttagaaaatcagacttatatcatatttcccttcttaacttgtagctcagtggtgcatttcttaccctgggggagaaatggaacatggactatatttagtatttgaaatcagacacatgtgaatttaaactctggctcagagtggcaatggcaaattattcaacatccatacaccctgatcactcccacatttgaaaagtgacaacaatactacttccttcacaaataatgcaaggatcaatgagataacaaatcaagaagccagtagtgtgccacaatttaggtagtgttgacattacatgaagaaatgaacccatggtctcctatgctctccttcactcctctctagttctctctctctctctttctctctctctctctgtctgtttttctctctctctcatacacacaggcatgcatacatacactttggaatgtagctattttttgagcattgttttattattatttaatttatttatttatttattttttgagacagagtctggctctgtcgcccaggttggagtgcagtggcgcgatctcggctcactgcaagctccgcctcctgggttcatgccattctcctgcctcagcctccctagtagctgggactacaggtgcccgccaccacacctggctaattgtttgtatttttagtagggatggggtttcaccatgttagcaggatggtctcaatctcctgacctcgtgatctgcccacctcggcctcccaaagtgctgggtttacaggcgtgagccactgcgcccagcctgttattattattatttcaaagaccttctgatcctggctctacctgctcattcatggggaagatgcccagacattcagatctactgttccactgaggagtttagaggctgctttttctcccccataaaagagaatggggactgatggaaaaattttaggtagaagtctttgatgaaaagaagaaatgaggaaaactgagctggaaatattcaggtatatcaaacaagtggaaatacctcaccctcataaagagaaccatatcctgggaaattttctttcttacctgattattaaaatccagaattggaattctgtgattccagtataatccatccatcaaaaccaaactactatgtaatcagattagaggtggccttcctgctgatgagccatttagagacggttgaatgagttcttggaagtctgctaacatgcacttaatacatctgaagattttaaaacgttgcagcaatgtattgccagtctacctacagtaaaataaaaatgcttccattaaaatacaggtatatttcttttatatagttaacttatttttcctaggaaagttagatgtaaagtgattatctatacactgaatcatatttcccagagatcatcatagtaagattggtatgcatttctgttaaagaaaagcaaaatgtgtcttaggacataacaatcatttaatggaatatctaaagaatgttggtcaagcattttgaacaagatgaagatggaaccagggctgggtgtgggcagtagactgttccaccaggagccaaagatgacagtgaatatacacttgacatcagtcaccaatgacaacctaattctacgtggcatactggcctggatcaatgagtctcctgaagttgaatttgacaaagatggaacaattgtgctcaggggctgcttattgttgctttatggtcatcctcttccctggctccattgccttgaagaaggtgaaattccatgaatacattcataacttcaaaatactacaagcagtatttttaagagggtgggtgatgacaaagtaatttctgtggacaattaggaaaaggaaactttcaggacaattttgaattcattcagtactttaagaagttttttgatgcaaactgcaatgaaaaagactataaccctgtggctgccggacaaggtcaagaaactgaagtggctccctctattgttgctccagttctgaataaaccgaagtaacctctcagctctagcaatccagctccacataggaccatttccacaaataactgcagcttctaaggctgcagcttctaaggccccagggtggtattaaaaaatcctggtgtgggcagtaggagtgctgcagggtcaaattgatgctagcaggtcagtgtattgaaacttggcattgaagccttggagaaagagatttctacttcagaaagctaaggaacaatgaattgatttgccagcagaacgaggggcaaaatgaccctgtattgcagaggactgtagacatgctctgtgccacagatgaagacttcgtgatatccgatgaaaggggcccacaggaggaacaagaagagttttaacaacccgaaccaggagagccttatccgaattctgcactccaaatcttgtgcttaactgttaaatactcccttttattattcttagaagactcactggtttctttttatgagcatgaagtacctctttttaaagtgcattttgcagagtttcactccttttttggtgagtttgagttgggagttttactgtgcagcagagcaacattaacatctggttagttcacctgggaaacaaagaggctgaacatggggctcaccatgtgcatgtaggtaacattgattgctggagaaggtgtttggtaatatgccgaagtggagaccttggtacagaaatgtgaagactgaattgaattttaacctaatgtgaaatcttggtagagaattttgtaataagttaatgcctaaagaatatttaaaatatgcttccatattcaaaatatcaaatgtaacacatcagaacatcttatgtgtttgacattgtatgttggaaggaagggccagacatcagaacctttggaacctggtgtcatcacaggccttacagggctgcttgcaacctcacaggcctagactttggccccaaaggaaagttctaaatgttgctctgtaaatccatttggtgtcattgaccaactgcatccaggctaaaaagcaagaggtattgttgcctggatgaacacagggtgtgtttcagccctgagatttttgggttgaagagctctattttcattgaggatttctctggaaatttttccaattatctctgaaatttctatgtattacgcttttttgggaaatgaggtgtgtccaattctttcatctaacaacgcttttggggatttgctcacatctctgagatttgaatggaggttgtttcccattttaccatcctttagttttacatttaacatgtcaccagtggtgaatccatagaggtctgcagcaaacttgatccttgcctcgttggaggaaagaatttggctgaggggaagaagtaggtttaaggtagagggagagaccgaggcaagttttagagtaggagtaagagtttattaaaaagttttagagcaggaacaaaaggaagtaaagtatacttgggaaaaggccaagcaggcaacttgagaggtccaagtgccctgtttggcccttgacttgaagttttatacattggcctagttatggcctggttacggcgtttgcatctctcttccctttattttcttttggagcaggctgtacacatgtgcagtgatctgccagcacttgggaggggcaatatgtgcagtgtgtttactgaagttgtgtgcatgctcacttgaggcaattttcctttaccagtagagtgttcctgagaaggtcatatgccagttaaactctgccattttgcctcttaatgtgcatgcttgagctcactcacccaattcctgagaacttatcaggaagcctctgatcaccaggttcaggtgttttttttttttaagccattaggggactgtctttacctggtgctggctaagaccaattattattttagacagtttagtaaccacctgaccgtcatctgatggttgcctgacattcctgagggaggggctctctactgccttgctcatgtctgcctaactacctactctaacattgcttctcttctctgctccccttgcccactggggacacctctttgggttcttaaagtttgcagcttggagttggaagtgcagcaggcaggtgggcacactgcaaattctttgtggacctctggcaaagggaataatcagggaaggctctggttacctctgcaaagctgggatgtttttggtatctgctgtccacagctctccagttctctgaatactttatcagtacactaatctcttaagagataaaatttattagtgtgttgctaaatgttcattttcttttacagaaaatacagtaccatgtctgaattattcatatttaaaatttgttattccttaactctccctcatttgatttgtgcacaacctattccatccttttgtttggcagaagtttgcaaaatatgtgtcactcactgagattgttcagcccctgatgcatttgtattgatttgtgtctggtgatagcttgtcctaaaatgtgtgtaaaaagtaaacattttataacaaaattgttgtttaatggatgctttgtgtggaattcagagaaaaatccagattcagtgattaacaatgccaaaaaatgcaagtaaatagccattgttcaaatgacagtggtgctgtttctcttttgtggttttttagacttttgttacctaaaattctgctttattagcaactcatttttcacctgatgtttcttgacaggctttttttttctattttaagtagtttctaaataatttttttttatttcaagaaagagaaaaaagaatattgctcaatgcacctaatataaaaataaactataaaaaataagttagtttcatattggaactaaacctagcattaaaaaaaaaaaacagcttttctcaccagtaaacactgaacgtgagttcttcatattctactattctctgtttctactctacagcagaaaaacacgtaagtctggttgttcagaactgtctccccattatggaaattgttagttacagaaatgggctagtggattcaggttaaaaagagaaggatacatatacatgtgtgtgtgtgtgtgtgtgtgtacatatgtgtgtgtatatgcatatatacaaatatatatcacagcaagtaacaatcaaaaagttgataagaaaatagacttgaacctatggaataagaaagctagaatatctgaaaaataagtattcaaataatatttagcaaatcttttattggtttatgtttacattactgtaatgtaccaaagaacccaaaaaactacaattgtagaacagaacttaaaaggccacttccagaaaaaaagactttccttagtgttttcagctttcttaattcttgggcacaaaattttctaagtctttgcgcacttatccacaaccactaattacaatataaactaatataaaattatgtcctcatctatgaaatttaatatgccattttcttagtataaatttatttctagagcatgtattattaatgtggatctttgtagaaaaaaattgtggtacaatgtttattactacatacccttatttttttaaattgtcttatctttcatataactttttgctgatgtcatcatgtgtaataacatgattttttaaaattttaatttagaaaaaatatattcctcttgtataaatgagaaaactaaaggaaaagagtatcgaaaggaaaacattatagaaatatcagaatttagaatcccaattcttaagattttttattttctgactacttcaaataatatcgtttgcctttctcattattcatgggtcttccaaatatagtgtctggccttaaaagttgtaacacaaaatcttctgtctgaaatgtagtacaactgaatgaaatttaaaaccttcacgtaatatatgtcatatagaactctcataaaatatcagtatgatgtgataacaaaggtcagaacaaatggaagaaaaaccaatgtatgatttctgaaaaccttatcctgggaaatgagaaacagactataattctttacttacatctacattatgcaaaagtatctttgatgtgaaaaaatactcagtgttgttcattcgatgttttatatgttttggtaaaacattttgtgttcacccttttaaatattccaagacacactcactggctagaatcaatgggagagaaagaggtgactctataactaatataatgttaaggaaactgcacatatcttggtatgtttctgatagtgcataattccagaaaatgtcttaagcattgcataattgttagaaacatgaaatctgcagccagcaagcatgagttccacatctgaccctactctttccagttgtgagaacttatttgtattttaaactactgaatttcaggatgcctctgtttattcctgtttctttattttttaagttgacagtatttgtacatattcatggggtacatagtgatatttcaatacatataacatatactgatcagatcagggttattagcacacccatcatctcaaacatttagcatttctttgtattgggaacattcaatatcctcctcctacctatttgaagttctataatattgttaagtatagtcatcctacagtgctatagaagctataacttatttctcctgtctagctgtaattttgtatcctttaacaaatctctccctatcttccccagcctctagtatcctttgttccattttttacttcttcctcatgttttaaagggaaatgattacagtatctatttcattaggttgctataaggaataaatggtttcatagctcttaaaatagtgcttgacaaaagtaggtactatgtgtagatttttattaaaaaaagtagatactatgtgttattattatgactaatcttctcccaaaaatatgtttggtaaatcatttcataaatttgtttattcttaatttattttgaattagaatcatttgtataacaagttggtatattgctgagcacatctgtaaagtaagacctgaatatggatttgataattgaagttgacactcatcatagagatttatgaaatattgtgcgcttgattgcagtaggacaacttactatggggaaggaatcttgtaagtatcttgcctggccagtatacatggaagttggtacacatggaagatataccatatgtatatatcccgatattgataatgttactttccaaaagtggaggcactgcaaatatgatgagaatgtgactagagcacgcacttgtttgttctttctgtccagcattaatttcccccttattccagtaatagtaactcaacaactctgctctttgtcatttcctggtgtttttttcttttcttttctttcttttttttttttttttttttttttgagactgagtttcactctgttgcccaggctggagtgcagtggcatgatcttggcttactgcaaactccgtctcctgggttcaagcgattctcctgcctcagcctccccagtagctgggattgcaggtgcccatcaccatgcccggctaattttttctattttttagtagagatggggtttcatcatgttggccaggctggtcttgatctcctggcctcaagcaatctgcctgcttcggcctcccagagtgctgggattacaggcatgagccaccgggacttgtctgtcgttcgttccttccttccttccttccttccttccttccttccttccttccttccttccttccttccttcctccctgtctgtctttctttctctttctttctttctttctttctttctttctttctttctttctttctttctttcttttctttctttctttctttcttcctctcctttctttccttccttccttccttccttccttccttccttccttccttccttcctttctctctctctctctctctctctctttctttctttgtcttgctctatctcccaggctggagtgcagtggtgtgatcatagctcactgcagcctcaaatgcctggtctcaagggatcctcctgccctgatctcccaaaatgctgggattacaagcaaaagcaaccttcccagctccaactccacttttcagaaatgcttctgtaccacaatgataaccctgactactcataggtttcaaatgaggactcttccgtgtttcaaggctacagcatgtggcttaggagaggccaatcaaagacggcattaccctggacagagtaattacttcaggatgggcatgtgacccaccaggtgtcagtgagatgcaatgagtttttcggagtctttcttcccactgaattgaggtctggaaggttgggaggctagagctgctacagcatctagctgtagggaagctacggctaagagaagaatctttaggaaacttatcactatgaaagtcagaacaaaaggatacagcataactatgagctggtaacatcagctgagctcttgaatcatcatatgcctaagactagccctggacaaaccatgaaccaataaactcctgtctttatttattttttacttcaattagtttgagacatctttttctcttacaatcaaaagttatctgctaaagtgactaaaatattattttgggctacatcttccccttgctcatttccaacaaacacattttcttcttttctccaaatcctttattttagattcagtgggtacctgtgcaggtttgttatgatggtatattgcatgatgccaaagtttgagggcacgattgaacccaccacccaggtagcaagcatagtacccaataggtagtttttcaacacttgcttcccccaccctatacccacttatagtccacggtgtctcttgttcccatgtttatgtccatgtgtacccaatgtttagctcccacttataagtgagaagatgtggtattttgttttctgtttttgcccaacaaatacattttccaatgaaaatgatgaagccagaaagtatgaaatacattgatttaacaattagaactgttctaattataattgataccggtgcctgttttctttttatgattgagtaatgatcacagatttttgctatcagattttgagtttgaatcctgcctgtatgatcttaggagacttttcctaatctcagtgtatgtccttacttcagtttgcaaataaggataatgaaagcacttatccaatagggttgtgtggaggatgaaataatacagggcaaatgtcttagccaagtgactggcacttagtaagtcctcaaatcttcactatgatattaattgaaagcctagcattcacaatctcactggtttatcagttcttttttctggttgctttttagtgacttaaatcagagataaataagattggtgtctcactgggttaatattttttctggagagagttaacttgtgtggcacaggccatctctttattgaattagcaaagaactacattggatttaagattatctggggctgaggctaatatcaaatcctactctatgtttgtagttccttggctacaagcctacatacggtaattgctgtttaatatgaatttttatttttaacccttacatatagtaagggttaattgctatttaatatgagtttttatttttaacccttccctatttacgtcatgctttgcttaaggaccaagatatattctgagaaatgtgtcattaggtgatttttgtcattgtgtgtacctcatagagtgtacgtaaaacctagatggtagagcctattacacaactaggttacatggtatagcctattgttcctaggctacaaacctatactggatgttactgtactgaatactacaggcaattgtaatgcaatggtatttgtgtatctaaacacgtctaaacatagacaaggtacagtaaaaaatatggtattataatcttatgcgaccactgtcatatatgtgatctgtcattgaccaaaaatgccattaagcagtgcactactgtactttctctttactttaaatttgaattctttatatgggttgaatgactttctgacatagcaaataaaaagcatgaggagaagcattatctgttaacaaaattaacacttaaaatcaacaaagttttaatgtttcgttccaagaaaagcctgtggaagatcagttccacaactgagagctttgggctgcttcagacatatgtctgtgtgtacgctgtgaaggtgtttctcttcacagttccccgccctctagtggtagttacaataatgccattttgtagtccctgtacaggaaatgcctcttcttacttcagttaccagaatccttttacaggaagttaggtgtggtctttgaaggagaattaaaaaaaaaaaaaaaaaaaaaaaaaaaagatttttttttttttaaagcatgatggaattttagctgcagtcttcttggtgccagcttatcaatcccaaactctgggtgtaaaagattctacaggggtaatgttttattattcttattatgcttattctctgtgatgcttctctacctttacagtagtagaatccttggggaaatctgcagagggaccactttcattttgaagctgctggctgcatgttttagcatgtctcttctattagagaatccaggcatggcagtttcctcccccagtgtgcaaggaccatcttcatgcctatgtctgtcgctaggcatgagggtctctaggaatgggtgaaaaaaatgagggatgttttggaggcactataatactggggagggcagtctgctagctggtagctgaaaggtcctggtttacttcaacattttttttaaataaaactgtgcagtagtttttgttattttaggtttccctctgttttatctggtgtatgctgcagaagtgaactgcataacacatttcactcttagaaaatgcatttcatatatttaaatgacaactctgactcctatagtatgggggtgaaatagattgattttttgaaaagatggaaaaaatagattaagtgtcatttaggagtcaagatgttgattgtattgtttcaaatagcatagaaactcctgccactggttcagaggtaacatttggaatgactgatgtgctcttttgagttatctactttagtctggtgaaatatgatttgaataccagtaaaaatctatttaggttattgcaaagactcatataataagagtttgcttgaaaagataaactatcgtaaacttattattttttctcaagtccaaattctgatatggacactaagactgtgatgctacgtttttatgaaatgggcataatttgaggaaattggctatctctacagtatttcctaaggtactgcaaatgtgttaatttaagaagcaattacactagctagcagcaggttgctaagccagggggtccgagctacagacctctgatgtggatgacttcctgcccggtttggcaggagctgggtcccttcctgtgcagcttccgagcagttccaaacacaaccaggcatcttctgctccttcccactccctttctttcaagcaggatctacctatgtttccatagcctggcattttaggatttccagagatttttacccagtgaattgacttggaataaactcttaaatgtgataaagtgagccaaagggcacgtgcaaagacttggagagatatgtgcaagatcctcagctcaggttctacaatttaatatgatgtttcatctcttgttttggatctctgctattttttcagaaagaatgctgaaaaagatggctgccaaaatattcacacaagtgaaacagcaggcacaaaccagtatcctccatttctgagctctgaattgcatattttcccacaagtagcacaatcacttaaaacattttgggagtgggagattagattacctcatagacacctactgaatgtgagagatagatctccatgtgagcaaatggatgcacatatgagattccctgaataccaaatacagctttctttgaaaatattatagctatctgagtttctatgctgtatctcagtgctcagtgtcatgtaactgacacaatcaccttttattctaatggtcattgctttttttccctccctgtttctgtagcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaagtgagttcttagtcaagttgccttcactgcctatttactaattggttctgggctagtcccagggatgatggtgaagaaggctggcctccttccctctgtctaaagtatcactaagatgctggatgggcctgaccgtgtaatggaccaatgatcctagaagtcttttggaagcactcatttgaacctgcatttgtgagacaggcagagaactggtgaggcatcctccagcgcgggaattaaggaaggacaaaagcctattcaccttcttgaatacaaattatatgcttaaaccagtgtaaattgaccctgattccctaataatgttgagaagcaaaaactgtaaactaggagtctatttaaattttattttttatatttgcaggagtagtatctaaattcctctttatagtctctagctctccataagtcactttgatcttcagtgggtttaattattcctttataccatactttctcctttctattgctctccacagaaggaataatagcaggtgacttgtaggtgccaaataagattctgagcaaagaacacacctggaaaaccttgaagttctcatgagaaaattttctaaccaaaaaaaaaaatcaaagcctcaattttgtgctttatgtgaattataaatgcggttttaaaatacttacattaaaacttgataaagttgctaagaattcctatggcattgatcacaaattttcttaataatcctcatgtcatttatcaaatttaggaaagtttatagtgctcagaaaaaaaaagcatctatcttcatgtcatatgatggtaattattatgttatacactattttacagggcaatatttataaataatggttttacttttctcttaaaatattcttaatatatattctaagttttattttatgtgttgtgttttctttttcagacgtttatagtattgaataaagggaaagcaatctctcgattcagtgccacccctgccctttacattttaactcccttcaaccctattagaaaattagctattaagattttggtacattcatatcctttttcaaatcgtcacttaatatgattttcttctttgaccaagttattgagctacacattttccaaaatatctgtggttggcaatgttatgtgttctttctttttctttccttttactcaatcgttagcatgttgcaaaatgagatcacaggtaagtgaattactttcccccgtcttctaagtgtttcttctctacccaactcactattacttctttcttctcttttcttctcccttacgaattgcttgccacatcccaagcctttctcattaattttgaccatgttaccaggctttcctcctgtaagtcttcaatttacaatgttaggtaagggagtaaactccatgagctaattttctttactgcttttacatttggaaaataaatatacataatctgaattacaattttgtatgtttttggtctgaattttatgactttcttctattttagcatttaaaagctttgagttagtaagcgtttacaattgtgtctgtaggtataacaccctttcaaatacttttccaaatttgtttcgcaacagccttcttattgatcttttgctttctattcttttcctttctttttctttttatcacttgttcctatgttttattgaagtcacaagtcttgctacaattatccctctccaaaggattaatgtctatctatccatatacattctcattttattttttatactccttattgaagcacctgctttccaaaaatgagattgatgacatcttggtgggagatggcaatttgattgattccttgaaattaaatagagtagttggaaatgagagattttattctaggccagaaacaagtcttgttgacagccagtcttgggaatgccaatgaagcaaaggcttggaagactgaagctgtgtggggcaggggcatttacacgaagaacacagaagtcatgggaggaggagtgatatgcttcaggaatcagaggtaaggcaggggaactgaaattaagcaaatcctcagattaggacagcatgttatttcttcttcagaaagaatcgttgcttggaattccatgatgtggttagcccagggcctggtattaaggctttcagtataaatattctccacctttagcaggcctagaaaatatttgaattagataaggtatggaactaaattaagtagggaattcaggaaatgggataagcctggtacagggtacttatgtcatttctgggtggaggggatgtaaagcatgtccctagacttgcctcccagtatggcaaatgttgtctttagaagtgtaacattctgtaaagtctccttttaacctctaggttgttcctcttccccagtttaggtggatatcaacatcttttatttgatgtttatgtttcatgttttaagatttcctagtttcttggcattaccttaagcaataatgttttcttacctctctctgttttccaaataagagaacccagtagcatgtggggaaaaagatgtctttgagttagcattagaaataaataataaagttggaatttatatttgggtctcatgattataaattatgatctattattatgtttcaagcatttgtaatctgtgcagtgaataaatctctgcataaactattcattatattttaaaataattgtatgttccttatgcaaacgtaatttatatataaaattacgtggaaaattctagcctagaactagacttctgttcctagtagacattgggaaaatattcattaaataaataagtgactagtaagtcagagattagagaatcagatacaaaaaaagtgaaaaaataagtttgaatggatcagaaaaaatctttcttgtcgtgcatctgaatgatgagatggagttaagaaaacccaatatatttgttttttacaaaagcagatttttgttttaaaacttttgtaatagaccatggaaaatctcatgaaaactattgtccccacttgaaaaaaaaaatcctaggagattatgaatccccatttaaaactccctggaaaagagactcctggtggtagagggtaagggcagtttaagaaattctgatcagagaatatgagtactaaggacacagtttgtgccaggacctctctagatatccagatacaacttgaatctgtgggcttgtatttgcttcctgggggaaagaactcaccctccacaggctgaagtcattgggaggcctgaggagatgcaggttgaaatgtgaagccagccaggcaaatctattaaggtatgccaagttgagctgttgattctctgagaagtttacagatggcttagagtcacaaatttattccaatgtagaaaattagattttaaaaagtctctaatttcctgacttaaagtgttatatttcagatgtctcaccttggagcagagatataacaaaaggcagtgaggctaatagtctaagatacatgaatccttccatgttttggtgatgctggtgcaattgatcaaatagcccagtaaagttagaggtatatagatgctgtagttagtaactgattttcacaataattttgtcctttattcctcttgttgcaaaccctagacttaaatcctgattttctgacttcaagtacagtgtcttttactgtaagttaaaaatgcttggagagatggtcatggttgtttggccacagttgggaggtcattgtatatttattaccactagtttataaaccaacaaggagccattcatgttaaataagtttttattttaaacttggactaatacctctatttcaaacaaaaaccttgacttgtttctcaaagagctgttatctattaggagctattgtgtatcaaattagcttttttaaaaatttattttggctgaatgagaaattatgcttgtgatatttttaccagggtgcattctgaaaactgaaaattcttttgatgtgcctagtgtcttatttgatatttaaataaaacatgatttattttctagataacaaacaagttaaaaataatctatgttcctaaagttccctaccaagcttttaaatgtgtttcctgtcagcttttattattttaagttaatatatgcacactcctctaatttattttgcatttgttactcatttgttcatttgcaagtacttactgagtatctaccatgtggtagatattcttgtaagcattgggatgcaacattgaacaaagtgaagttcctactctcatgtagttacattcatgtgagtgtgtgtgtgtgtttggaagaagaaagacaataaacaaatacgtcgattgggagctagtgataagtgttactaagaaatataaattagtgtcaaagggaggagtgacaaggtgttgttttagattgtatggccagtaaaatcctttctgaaaaggtaccagttgagcagagatccgaaggaagcaaaagagtgagatatgggaatctagggataaagtcagttcaaggagaacagcaagtatgaaggtaaagtctgaaggtggtggtgcaactagtatgtttaacaatcagcaaggagacctgagtggctggagcagagtgggaaaggcaggaagcgagaggatgacatcagtgggagtgaaggtcaggggctaaagttgtaggcgaggtgcagtgggtcattgtgaggacaccacatttactctgaatgagactccaggagggttttcagtagcaggatatcatgacttgactcacatatttaaaagatcactctggctgcttcatggagaatagactgcataaggggaaagagtggaagcaaggagactgggaccgattgtcattatgaaggcaagagagcatggtggcttggagtatggtggaaacaatgaggatggtaaaatgtcatcaaattttgaggtattttgcagaagagctgacaggatttgctgagcaattggttgtggtgtataggaggaaggcagggactaaggatgattccaagcatttagtctggtcaagaaagaaaaatggagttgtgttgactgagcttggggcaacttgagcaaaccaatttggtgggtaagatcaagatcttggttttgaacatgttatgtttgagacacctattagacatcattggaggagttgaggagttaggtggctgtgcaaatctggagttcagaaaagggctgggctggaggtgttcaactttgggagctgtcagtgtacagctggtatttaaagtcatgacattggactaggctgccaaagagctgagaccctccctccaaatcacactagtaatgctgaactacctatcatttgaaaatggtaggaaaatggaaaacataggttttggtatcagaaaacgtagattcaaaccctatctctaaaatttactttttagctatatgatcttagtccaagttactccaattctttcgaatctcagtttctctatctgtaaaattataatcacagcttagacattaataatgataaaatgtatgacaagtatctagcaccagatcccatgctagtacttagtaggtactcaataaaggatatctatgacagtaatagctaaaattctagcagcaactgctgtaagattagcaaaaaggaaactctcatattccttaaggaattgcacaaagaactttatagaaatccctactctgactctgcaaacaaaatctttatatagcaccagagtttagacctgcaactgacccaaacaatgtggtcagttctgtctcattttgtagatgagttcactgaaacccagagatatttagttttttctaaggctacattttctatcagtggcagagctaaaacttcagaccaggttttttgattcttggctctttgcattttgcatccaatagaaaacaaatgatttttaaaccctcggatttaatatacttggggcattgccagtgttcttgttttatgcatttcaaaggtgcttcttagttgctccaacttactgattcattaaatagtgtccatactgagatataaaatatcatggttttccatgaaaagaaatatacaggtttatatgaaagcagatgacacaacaatttctctttcttttgttttcaatgctcatatgttatcatttagttatctactggcaaataggagtttgttcatattaaaattaaacaatccaatatttaacactgtatatgtgacatttactcgatttttctgctggctcagaaatatgcactggtatgcagaaaaagacctattctattctacttcaaattatccatttttacattagaaaacctctaacatcaggctatcttctacttctagtttatatataggttaaaaactcctctgcaacttctctggatattatacattattacaaagtctctgaacagagcataatgtcttttccttcctatagaataacaaagaaatgtcctataattttatactctataaatgagttattaatggtaagaaaccaataattattatcttagtggataatgactgtatactgtaagaaaagtattatccacatttatataagaaaactgagcctcaaagaattaaacaaattgctgaagcccacatggctggtaagggatgtatctgaccatggttcattgctctaaatctcatggtgcttcatcctcgctccacggagacaggggtgggtgtgccagtgttatgatgatccaggctccatgtcaagggctacttaaacaattttcactaaaaacttgaagaagtgtttcttcataatatacacaaaggaaatattttacatttgccaactcgcaggttagtatcaatcaacaggtttacccactgttatgtatacctggcataaagaaattaatagattaaaaaacatctttgtcccctgatattataaaaggtttatctgcctctattttattttacattgaaaagttcttaaagcaatattgttccaggatacagtgttcttttgaaaaatgtactctatgacttggattacacatttaaaaaataatataggatgtatgcattttgctactagtttgagccttttgaaatctgctttgacgtggggtttctatacttttttgatgcatggcatcaccaatgcaaaatccatacctacattaaatacttttgtatttgagtttttgttatttgagtttttttttttttttttttttttgagacgggttctcgctctgtcgccctggctggagtgcagcagcgcaatctcggctcactgcaagctccgccttccgggttcacgccattctcctgcctcagcctccggagtagctgggactacaggcgcccgccaccacgcccggctgattttttgtaattttagtagagacggtatttcaccgtgttagccagggtggtctcgatctcctgacctcgtgatccgcccgcctcggcctcccaaagtgctgggattacaggcgtgagccaccgcgcctggccatatttgagtatttttaagatcatctgaaactatttcagtcactcaccagaatccaggaatttgtaaagtatgtgactgatgaaataaattaacaatgatttagaaacttagtgaattttaagcctttctatttagagatacctatcaaaccacaagcgtaaaaacttgaccctagttatctactatttttctattaaaagcaaaattgttctttttatgtatcagaagttttaacttaagtgtatacttttattaaaatgatagccatgaaataaggaaaatgcctgttttcgacttattatcagtgactaattagaaaataattatttctcttgttaatgttgaaatatatattttacttttttatatataactaaattataccactataaagagtaagtttttaagtgtcataaaaccattgccgagtccataatgcagcataattgcataaggctgttaatttccaccttatatttttcttatatttttaccctcaaaaaatgtagaaacttgtgtaaacaatatgtatatatattttagacagagtctcactctgtcacccaggctggagtgcaatggcgtgatcttggctcactgctacctctgcctcctgggttcaagtgattcccctgccttagcctcctgattagctgggattacaggcatccgtcacgcctggctaatttttgtatttttagtagagacagagtttcaccattttacccgggctggtctcaaactcctaacctcaagtgatctgcctgcctcagcctcctgaaatgctgagattataggcgagagccatggcacctggccaacaatatatttgaagacaaactttatgctgtatttttaaataatttatcagaaattgtttttaaaaactccatttagtaacaaatgaattgcaaaattaatttcattagtcaactgacactgtgaaatagcaaggctataatggtgaataatatagacatgattcctgtcctcatgacgctcagagagtagttgagaagatcatcattaaaatttgtcattagaggaataatataagggctgctggggtatataactggtaataggtcaatctgagtactaaagaaaaagagaggtgacatttccaagaccctaaagtcagaaacagcatataaaacattcataacatttgacaacctaaaataattacagtattatccaaatggggataatgcaatgaagagaaaagaaaatgaaactggagaagtagacagggatcagacctcctagcgtcttgactctgtgttaagacatttgatcatcatcctaagagtaatagaaagctaccaaaatgatgcatattacatttacaatggtcgtgttagcacagtatgcagaatggattaaatggagccaaacatgaatttggaaacatcagtttagaggagactgcaataatctagatggactattagatttgatgctagtcttgacagacacttggaccatgcagtgatatggggatggaaaaaagtaactgaatccagagatagcaggcagaattgacaatatttgatggttaattagatatgaacgtttaaggggagacagaaatctaagatttctcataggtctctggctatattatgcacattttataagacacagagacgtcaaaggagtaagtaattagcaggaatggaggggtagattaaaagatacttttcaaaagttcagttttagaattcaaaatttgaagtgttgataagatatgtaagtacagatgtcctatggacaatcaagtatgtggaattcagaagagcggtctcacttggagagaagtatctgagaatggtgggtatataatggttatgtttgttgagcaatgtttgttgatggactacactaggatgaggagagtagaagagaggtagatggcttacataccttatgtctttcttttcaaaaagaaaaatgccacatttcaaagaatacagagaaattggtgccctgaggcatgaagaagccagggaattggaacctcctgaaagcagagggaagataacttaatgctaaggggaggaaccatccttgttgaatgctgcttagaaagcatgtcaaatataatctcaaattatccttttgtttttagtgacaaaacgtgaagatgttgccatctttagaaagaaaagctggtaggctgaatatatgatagatatataagaaaaggaaagataagcagctctttcaaaaagtttggccatgatatggaaaagggagataaggctgtagtagttaaggagggttgtgagtcataggagagaaatatcctttcccctatgccaggacaagggagacgtaagcaagtttgtgttgctgggaaaaagccagtaaggtgggagccattaaggataagaaaagagagagataagagattgctgaagtcttctggaatggggaaggtgtccagaataccggtggagggattggaaacctccccactgtaacaggaaggaggaaagaattggtctcaatgtggaaaagtttgttgatttgggggtgggaagtggaggcggggcattgtgatgctgtctcttctctgtaaagtagaaaataagttttcagcttgaaatggagccggaagaaagaagagggttggaagctggaggaaagtggagaatatttgaaatttttctttgcagagagtgggagatggagcctagtaggaaaatacaggactgtgttgaggaccactgaggtttgtgaccataaatttagaatggtgccaatctgccacggggtgttatttttccccaatagggctcagcagaccaacaagcacaggggaccctctagttttatataccaatagcaagtcattctttatttaatttagttttttgtttgttatagcaataaagaaaaattgtgtttctttgaaatggtattttgtgtgtgtgtgtgtgtgtgtgtgtgtgtgtgtgtgtgtgtgtgtgtgttctctagattatagtgccctcacgtggctgatgcacatcatttacttatgagtgttttcatatggatgaaaaaactgagaattaagcatgggccttacagcacagctactaaaattaaaaaataataattattattattgttttgactaaaaccagtactagatgcaaatgaactttctttctagatcaacacaacattgtccagttgtaatagtgttgatatttcattatgtgtaagtaatgtgatcatttatagtaaaaacattaggagtgagaaaagatatgaagagcacgtatttcctctctggaatttctataattgtgtccagattcacaatgataaagagtgccactttacataatggcaactaaatctttattatgctttttattaaatatgaaagtcattactattatctgatactgaatattttcttaaatagttgttttggtttttgtcttttgctgttttttaatgtcaaggaaagtgaagggcattggctatacctgcaaagacaaagtgggatagggtgaaaccaacccaatatttgtaataaactgtgttctgtatgatccaggaagcaattcattgagcatctattacgcactagggccattaagttgaaggagacttacatattttaacaaatttgatcttcatagcaatcctgttacatgattactttttcttacttttataaccaagtcaattgagattcagggaacttaataagtaattttgccttagttacaaaccccagaaatgccagtgctaacacctgccatcttctctcatagttcaggattttatgagcaattacagtatattataccctctgtttagaaaggaccttattataagacattccaccagggtaacttttagaatgatgttataatacatttaattaattacttgaattgtcttgttgaatttttgccagggtttacatatgtgctgaacttgcagttttaatgttcagttgagtctgtcgttaagaaaatttaagttgataaattattcactgatgaactactttctttgcatttaatctttttaattgctaaaggtacctaaatagcctcaaaatagttgatggcttggcctgaagacaagatctaaatatgaggttgctgagttatagaaatggcaaaaaaaagggtcaataatagaataataagcaacaaaataatagtaagcactaaagttttaaacttcatggtggtgaaggcatggtagtgcataaaagtaagatttttccattgaactttgtcttccttgacgatattctactttattcaatatgctcattatgtgcacgattcttaccaactgtgtatttatgaccatgagtaaccctccagactggacaaagaatgtggagtaagtataaatatttttcaatattgacctccctttatgtttcatattgtgcttttaacaccttgagacctcctcaatttctttaacaaatcatgctagctactgttaaccagaccctgattcaaattcatttctgtcactaaatgtcttctaggacaaagcttgtagtgggctcacttagttgtgtaaattactgcagtagtttgactgctattatctgcagccctttatcttctttgtgagtcttatgttcttttgaagatcaccagtgatttactaatatctactgataaaaagtatacctagtttttatgttccttttttaatgactaccacagttctgtgttactagtatatgtttgatggcttttaatggtgcatattttatgaaatacaaatgcttcactcatttttgtattaatacctatttgctcaaatcggactgaatgccagtgtatttcagattatgtcttcatatagagccacattatttggatcctttaaattaattaatgtggaaaatgcaatatacatttatttacagtcaatgagaatgtcttttggaatttaatgtttcttttttgacttaagccccacctaaactctatatcgtagggggaccaacctggaagtgtctaatttttgtttgctgtttatgtcatctttaagatatgtacttgtaaattaaccactagatttttaatgtgagcttggctattttctctcaggtatacctttacaggaatttatacttttgaatcacttattaaaatacttgcaaggggcttttgtttagaagatttcacatttttacgggatccatggaattggttggatttcacagtcattacttttgcgtaagtatcttaatacattttctatcctggaagagtaaatcactggtgggagcctatactatattttccttggtggcttgccttgacagaccaagcatttttcttagtaatcatagttttcttccaatcaaattatccagtttggagaaattaggaactatcatagtaaattacatggctttggtttcaattagcactgtaaagtaataaagtttcccaaataacagagattatgattgatgacaatgccattttcctcttaattgggaaagctgatggcgacactcatgaaattaaaaaggtcttgatgaaagaccaaggaagacgtagatttccctaaattctgaataactctgatttaattctacaggtatgtaacagaatttgtaaacctaggcaatgtttcagctcttcgaactttcagagtcttgagagctttgaaaactatttctgtaattccaggtaagaagaaaatggtataaggtggtaggccccttatatctccaactgtttcttgtgttctgtcattgtgtttgtgtgtgaaccccctattacagatatgtgacagagtttgtggacctgggcaatgtctcagcgttgagaacattcagagttctccgagcattgaaaacaatttcagtcattccaggtgagagctaggttaaacaccgaggctgactttagctacagtggtgctacaatcacagcttttgtgcagaagccttgttgctagttgcatattgcaaataaatatgtaaaaaagcaagaattggtacatcattttttggatggatttgattctttgctttttactcgttgctttctttaaaactattctaaatcagcctttgagtttaacaagtgttgcatgaggcatttgcagtaacaggctacatggtttgcatcctataacatcaagctttccgcatagaagctagactaagagacattcagactgatgcaaatttgacagtttaggcctaaaactggcaatcttttaagctgcagataaatgaaagagcaagggatagcatgagtgctgcatggggctcagatttcagatgtcttcctttttttaacccatactcaagcttgcagaattcacaaatatataacctcataattcatcgacttcaagatttcttactactctattcacatagacttttctaaaaccaataaggggttagggagtaagacatctgcaaataaaagcaaaatatttacacaaggttgatgtttaagcatgaataacaaaatcattcttttgctctaaagagtgtttggaaatacacatttggttcatttccattcacagttttctaatgaacatacaagttctgctttcattcattttcaccagctagcaggcttttcatgaaaatgttattcaatcacaaacattaaactaatattgttggcattctgcatgacatttttattttccaggacaagctcatgatatttttgccggtaaaatagctgttgagtagtatatttaaattcccccttctgattttgtttgtaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtaagatatactctataaaccattaagttgtttagttctctaaatattaaatattatatataatggaaattatctcaatttagatgtgaatcaagtgacttagactaatttaagatgatttaatacatataaaagagatatcaaatgataccttattctatttttcttatctgtccattgatatagtaaaagttctcatttgaaaatgtgttgtcttatactcatgttgaaagtaatttcatattatgccatattaaaaaatgtttatttggtagacattaatcaggtttttcagtcattttaataaataagtcagtagtttgaactattcagtgtattccactgaaatgtgttaagaagactgaggggaaataatttggccctatttggttgatgcaacatatgtattgagtacatatgctatatctgaaaatagagaaaccatttatcaagatgaaataagaatttgtgtgctcctcagaaggttaagtaaccctgatttagccattcacttattcatattctaattagtccctttagtgtcatcattgtattgtagttaccagtttagtttgattatatttaaggtatgaacatcagaataagcttatgccatatacttcagcatgatttcttaacattgagcccagcccctctgtcatttttcatatgtgtgtgcatgtttgtatgtgaatataaaaatacgtatgtttgcatgtgtgtgcatgttttctgagatcatctttgcaacttactgaagttatatgtcatgccttaaaaataaaaactagatagctctccatagcttaaaaataaaaactagatatactcagacaacatatctctccaaagaaacaagtttattttcttcatttgaaaggcagaaatcaagcaaaaatttcaaacaaaacacttatttacagtatcataagagggaataaatacctaatcccacttctcacaggaaattaagttaaaattggcgggaaaaaatgtctgaatctattttgagcctggggagaaaagtatatgtaaggtaaaatttatttgcatgaaaacacctagaaacaacaaggctttcttctttcttactttttgtgcccagcaatagactggcagctctttcttaatgtatcccatgcaatttgagcttatatttgcaatgaatgctgatataagaatgttatcatagtaattccttctgaacatttttctttttaacatagatttgctaaccatttgtataatcaaaaatgttatatattgatatttgttcaatattgtgaaaaatctctttagccatatatatttattagtttatccatctcattatgattgaaaacatttgtgagctttgccacctaaacagggtggctgaagtgttttacaggattttaatgattctttctattcctttctctttaaataggtcacttttattttttagaggggcaaaatgatgctctgctttgtggcaacagctcagatgcagggtaagtgatgcttcctactgagtttcagtccacactgctccatcagtgtcaataacctgccacctcccactcatccagtcccactcactcctcactcaaaaccctccataaattctacttcacggtgactctcagaatagccaggataagtgtagattctcacctctttcacacagtcatttactgcaattatttttctatgctaggtcacatctaatcttccaaattagttcaatgtaaaatagagaataaagcagtataatatgcatctgaagcttaatagaattcttaagcacatactttttataagtgtcatattttatatatactaatgtgttctccatagcttaaaatgtaagatctctgaaaataatgttaatatctgagacatggggagtatttagcatattttagcaaagtggttacaaacataaactggagagtctgcatagagtcagactttgactccatcatataatctcatttcttcttgccttcgtttccttatatgacaaatgggtataataatagggtttttgtgatgatgaagtggattaataaatgtatagctatttagatcgctcaataagtgcttgtaattgttattattgggatcatgcaaatgtttgctattaagaaacatggagctaaatcctaggaaaatttaaaaacacagttaattttctttatttagcaagattttagagccacacacaaaagtctaatgcactttctttggacgatgatactgtggacattagtagctaatacctgtagcaaaattcccagtgataataggctttccatttggctcctacgatcagtgctatgctgcctttatcttcagattccaatgataagtaaatcaattgattttcattccttgtttgtactgtactaaatgcgttacatacagtatcttcttcaatgtttgcaaatttgtgagacaggttctcttattagcccattctcacatgcgaggtgcctgaagattagcaagttaagtaacttgcccaagatcgttcagctcagaagtgtcaggcaagacattgaagccaggtctgcttgatcttcaaggtcctcctatgacatttttaccacacagtgtcattcactccttgcagcatgccccacctatccttttctcacttctttaccctgttcccacacttacacacatttctgcctcaagacatcctcagtgaaaatcaactttttccttacagacttttttaactgcccttaagtcccagaagatattaatcatgatatgattgcttttatatggagacataataaatataataatgacaattatgaatcacagaggaatccacaaagtagaccttatagattctgttattatataaatcagtccacttagtgctgagttaagtactgggtaaggtgagagaaatcggcttttttctagtgcctgtataaaacagacattggcatatattaaaacaggaaaaccaattagcagacttgccgttattgacttcctttctttcctctaacctaattatagccagtgtcctgaaggatacatctgtgtgaaggctggtagaaaccccaactatggctacacgagctttgacacctttagttgggcctttttgtccttatttcgtctcatgactcaagacttctgggaaaacctttatcaactggtgagaacagataaaatcatttttctgagaatcataaaacaccgaactcaagagaattgctgtagaatattttattacttagagtgtaagtttgtaacatcctatataaaatttattaaaatctctcttccattttgcagacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggtatagtgaacaagcatacggtcctttgtttttctttatctaaattctttaacctaaatgttgaggtcagtggcaaggtagttgacattagaaataggtcatatgtgtttggtaagtgctaggagcctgtttggttattaagaagttattactttattgcaatgatctctgtcaatagtgtcaatagtaatggcatcaaaaaatggataattataattgctttactgacatttttttctcccttgtgactccttgaggaaattaatgattaacaaaggcctcatgtactcaaacttgcagagtagataaacctacatgtcctcagttgaagtattttcttaggggaagaggaattcagttacacttgcttcttcattgcagtatcaccagaggtggtaagggtcagaaaaccagaatcaaactaagaaaattatttcattgagtctggaaaggcaaaggcttattcaatatttgttctcttttatataaagtgtacaaatgcaagtttgtgggttacatcagtaaatcactagtgtgtaaacatattaaaacattagcactctctgcctcctactctacaaatcctttaatttggacttgacaagccttcaaaataaggcaagaatttctctaattatatttgcttgacttaatggcattaactaatccaattgcctatttttgtcttttcatgtatggtgaatacaattcccttttattaccgagtattcctaaatatgtaataaaggtcaaagtatattgctgtaatagcaacaaaactactgttatactttacaagttcatgcagatgccatgatctaggattctcaaataaacactctgtattatgtctttgctgtgcatttcttagtgaaatacccaatttaaatcacggagaaaaatgtcattaaaataaaatacttgactgaattacatttaataattcagactagcactaaatttctttattgtgtgaaaatggaatcaaaggcaaatgtctaccaggtttaaataggaagtctttaattcccatattatttccttcttaaaatattgtttgaattatagaacatgttattatgatctttaagtgtcttgctcatattattagataattagatatcatagtgtgaggacagagcttgaaggttctcataaaagtcgtatgtatcatcttccatatgaatgcccattttactctttgattggtctaataacaatgtactgttttctaaaacacagaataaaatggagaattgtttttcaagattatcttcatgatattgaagctcaattaagcagtaacatgataattactttttaagtttatatgcaacttccacatactttgcgcccttctaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccaggtaaaaatattaaattacatgaattgtgttctcataaattttttaaaaaaatatgccagaatttaatggagagaaaaccgccttccacctggatggcacaatgctttcagagtagtgatgattatcaagtgttttggctatcacttcagagaatttgtgagttttgcaactttttggaatcccaggaaggaaattttagatccctctgggtttggaaaaatttgcggttttgaggttttcttaaagactgaaaaatcttggagaaattttccacatcaggaattatcagcagatggttcccatctcttcttaactattgtgcgtggatctagtgaactttgggttttctgagtgacaaattcccagaagtggaccagagactcttttaggccacctgcggggttgttcccataaggtgcaaacatcacttgccaagtgcattcttcatgcctttgtttcaaaggggactgaaacaaaatatctctaaaagtagccaaaactctcagataggcaggtactgagggagatttatgacacgaaataaaaagtggtgtttagttgtacttgattatctgtgtttcatgttaaacatgggacttgcatttgaagaatactgtgatttataaactgcaacaaatattcactggatgcctctgccttttgtactcatgcaagttgttgaaattttaaaatttagaatcttaattgtctttgaaattaccaagagaattcacaggaatacacagtacctcagaagacattttcaccaggagtgaaaccttaatacctatacagtaacaataacaattacaacaacaacattgataatggctaatatttatacactgtgttgttatttgcaatattaatgcattaagcctttcactgcaaccctaggtaagttctaatttagttaatatccccaatttttatatgtgaagagaagcagggggctgcattctttgttgagtacctattttatgcttggcattctttctacattctcataattaatgctaacagttctgtagaatagtattattttcattactataatgaaaaagctattttggatcagtaaagttaaatagttgcacaatgtcatatagcaatagaaaaagttattatgatctttgagtgtcttgttcatattagggttgttggtattcaaattttgcctggtactgaaaaccttaaagtttccactttatcaagttgcctatgaagaatgcctttaaaaactgataaggaaatttacaatataactttatttaaaatacacaatggcattatactttctcttttacctttttataatatagacaagctcacataacctcacatgtgatatatataaatttttttaggtcagccttattcatttcaaatccaaataacatcataagattgcatacttggggattaattcaaatttaacataggatctttaaatatcaaaatttacttggtctctttcattttgttgtcacaatcatgattccattagtagaaacattaatcaataaataggaatcctttaaaaggcaaaccccctgtttacagtattagtcattctgaaaaggaaggaagaaaaagaaagggagggagggaggggggagagagagagagaggaaaaaagggagcaaaggagggaggaagatagagtatttttgcctacatttttacctaagtttgtctgaatttttgcctgaagttgtctaagttttggccaaaatttgcctaaattttggcctagatttgaaacttcatatcaacttcatatcaaacacttaccacagagattctcttcaatttgccttatttctaattgaataaaactaattctaggcaaaatagtgaagcctgataaggctaggctctgtccttcctttttctgtacattttgttcatagatatgatattctcccagcagcctcttcttcatacctctacatacctccatttcccagctagttgggtattataagcaatcactgacttagaagacatggcatggctggctcatattgatacttgtttcttaagcagtctctatataaaaatagagttaaagactttattttgcttgataaagaaatagtcaaacaaatgtctaagaggatggagagggagacagaaaaagacagagggaaagggaaagaaagagaaagagagaggggggaaaggagaaaaggaagaagagaggagagacagaaaaccctgaaatcacaccaaccccacttggcaagccctgaaagtaatactgaaaatgtcaaaccagatagaagtacttaatcttggtatagcaatggagggcccatcgtgtctgttagatttttaagagtttgagaccccaaaatattaggaattattgttttgtgatgacatgattgtgttggtaaccatctctgtgtttgtattgaaaaactatacaatgcaagcctttactgcaagattataatttctttagtagagtaagtggaaatatgaattgtttctcagactctgatttgacttgttagtgtggtaaagaggggagaagaaagtcaagaatgtaatctctaaacaagtttcaagataatctggatttttttgaaacctttataaggtacaattgaccttaaatcattactttattatttatttgtgataagctaggagtttaggagttttgctttttaaagattggtttggtatggggaatatttcttactggccatcttttttgtgtgttacagcatttgattactatgcatttatgtaatgaatgtcagcaaaagaagttgatgctaacggatggggcacaatcatttctcatatagctgtcacatgtaaactacgtttttgtatagcttaattcatccatgatccttgagaaacatgcaaacttataacttattttcttccaacccttctatggctccagctgaatggggtactggcagttaaaatataaactcttactaaaagcgatagaaacattcttcattgcaaagcatgtattgtttgcctttcttttttagctaatgaggagcagtatgtcacacatcctgcaaatcccgtaactgttatttcctcatagctaattcgaagtcccttgttagaggagagaaaggagacacgaaaaaggatggatagtctaagaaaggctttaaaaaataactacttgtatggaaaatgataaaagaaaagaatgaatgttactaatgtagttaataggattaaaaagcatgggaacaacaagaggagagatgacttctgttgtgggagcagtaagtcttcttagaagtagttctaggccgggtgtggtggctcatgcctgtaatcccagcattttgggaggccgagacgggcagatcatgagatcaggagatgagaccatcctggctaacacggtgaaaccccatctctactaaaaaatacaaaaaattagccaggcgtggtggcaggtgcctgtagtcccagctgctcgggaggctgaggcaggagaatggtgtgaacctgggaggtggagcttgcagtgagccgagatcacgccactgcactccagcctgggtgacagagtgagactccgtctcaaaaaataaataaataaaaaaaaagaagtggttcttactgtaaataatgaatagaatcacataagatagtgtttaacatttacagacatttaatagaaactaacagatattattgagaaaaagtaattctttagctggaaagaaaataaaaagcatacttattggtcagtgtattactctgttttcatgctgctgataaagttatacccaagactgggcaatttggcaaaggaagaggtttaattggacttacagttccatgtggctggcaaagcctcacaatcatggcagaaagcaaagaggagcaagccacaccttacatggatggtggcaagcaaagagagagtgaaagccaagcaaaagaagtttctccccatataaccaccagatctcatgagacccattcagtaccatgagaacagtatggggaaaacccctaccatgatttaactatctctgaccaggtccctcccacaacagtgggaattatgggaaatataactcaagatgagatctgggtggggacacagagaaatcatgtcattccacccccggcccctcccaaatctcatgtcctcacatttcaaaatgaatcatgccttcccaacagtcccccaaagtcttaactcatttcagcattaattcaaaagtccacagtccaaacaaagcctcatcggagacaaggcaagtcctttccatctatgagcctgaaaaatcaaaagcaagttagttacttcctagatacaatagggatacagacattagctaaatacagccattccaaatgggagaaattggccaaaacaaaggggctacatgccccatgcaagttcaaaatccagcagggcagtcaaatcttaaagctcaaaaatgatctcctttgactccatgtctcacatccaggttacgctgatgcaagaggtgggttcccttggtcttcagcagctccacccctgtcactttgcagggtacagcttccctcctgactacttccatgggctggcattgaatgtctgtggcttttccaggtacacggttcaagatgttggtggatctattattcttgggtctggaggacagtggctctcttctcacagctccagcaggcagtgccccagtagggaccctgtgttggggctctgaccccacatttcccttcctcactgccttggcagaggttctccatgagagccctgcccctgcagcaaacttctgcctgtacatccaggtgtttctatacatcctctgaaatctaggcagatgttcccaaatcccagttattgacttctgtgcactgacaggctcaacaccatgtggaagctgccaaggcttgaggcttgcaccctctgaagccatgggcctagctctacatttgcccctttcagccatggctggagccgcagagatacagggcaccaagttcctaggctgcacacagcgtgggactctggacccggcacatgaaaccactttttcctcctaggcctccgagcctgtgacgggaggggctgctgcaaagatctctgacatgccctggagacattttctccattgtcttggggattaacattcagctcctcattacttatgcaaatttctacagcctgcttgaatttctcttcaggaaatgggattttcttttctgtcacattgtcaggctgcaaattttccaaacttttatgttctgcttcccttatgaaactgaatacctttagcagtacctaagtcaccacttgaatgctttgctgcttagaaatttcttctgccagatactctaaatcatctctcccaagttcaaagttccacaaatctctaggacagggccaaaatgccaccagtctctttgctaaaatataacaagagtcacctttgctccatttcccaacaagttcctcatctccatgagagaccactttagcctggaccttattgtccatattgccatcaggcttttggtcaaagccattcaataagtctctagaaagttccaaactttctcacattttcctgtgttcttccgagccctccaaactgttccatcctctgcctgttatccagttccaaagctgcttccacatttttgggtatcttttcagcagcgtcccactcctggtatcaatttactgtattaatctgttttcatgctgctgataaagacatacctgagactgggcaatttaccaaagaaagacatttaattggacttacagttccatgtggctggggaagcctcacaatcatggcagaaggcaaagaggagcaagtcacatcttacatggatggtgtcaggcaaagagagagtgagagccaagtgaaagggatttctccccataaaatcattagatctcatgagacttattcactaccatgagaacagtatggagaaaactgccactatgattcaactatttcccaccatgtccctcccccaacaatggaaattatgggagatacaactcaagatgagatctgggtggggacacagccaaaccatattagtcaggcatataaaaacctagatattagtaggtataaaataatggttttagttagttttgaacctttggagaggaaaagatcaaaccaataatacttaaatgatgaagttagtattctttccaataataattacataaatccaatgcaagctggctgaagtaaaaaagagaatgtattagatgatataaattatgtctgtgagtagctttaggcatggatggatccagaagctcaagacatgttaccatgaccatgtctttctccatatctgaatctaggttcctcccacatggtggcaagatgtcactgcagctccaaacttaaagccaatatttttagcagcctcaagtgaaaaagcaccatttcctcaacagttgtaaaagacagggatatctttcaattgaccaagcaggatttcatgaccatctttaaattacgtatcgaatacactgattggctaggcctaggacagtgcctggaacactgggagacccgagcctcaagtcctgctgcatccaggagttgaaattagtgtctcccagataacctggtctgagagttgaggattatttctcaaggctgaacagggtactgatttaaaaaaacaaaaaattaaatggttgtgatcagcctcttagtgaaaattaagtttttgtaaatattgccctcagatttcttgagacagagacaaaggggtgaaaattggggaataaatcatacagttatttcagcttgatttaatttattcatgaagaccatcataaaatatgcaaagggaagtggagaagctgccccgtgtactataattaaacatccctactagcaagattaattatatttcctccatggtaagatttgcatcagggtgtggtcactagcgagctcttactggctacatttttgacctcagaggatctaaaggtagatttgtgtttaattgttttccattgggttgttaactgaaattaacttctaaagaagggtctatcaacagtatcagttctagatgcccgtaacaggacaaaacattatggggacacttctgactatgttgaggtgtgggtaaagtaggagaaaagagagcagaagatggaaaatggaggaaggagaaaaagcgagagtgaaatagaaaaggtgaaccttgtagaaagtgccaaaatgccaccagcagtcatcagaggggtgctttcttccacatgtccaatgacttatccttgagtaagtcaatgactatgacacaatgaatcaaattctgtttttcagaatgccagctcttaactctcttcatctcatttttgtttcttctcttgttattcatagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagaggtgaggccaattaaaattgcagctgatgtgaagagagttgtgactggtgcaggcaggagtgtttttccatttccacatctaagaatttgttgagtttgttgcccaaaggctgggagtttgttcaatcaagctgttaactgtcttgtgaaactgttctattcagacttttctacaaagtaattaaaaacctaggttggctgtcagagaatataattagaagtaatctttcatcattattactatggtatgaaactcgccaaaaagcaaagcaacaatttatcaagcataatgtttgattaatatagttaaattaaatccaaggaaattaatgctcactaattaaataaatacttaaggattttgtgattgttgttcatttaaaaggagatttgaatacttccacttgcagtagatactattactaaatagatttaaatcccatagtacaacattgcctctctttgcaggtcagagtgttgtaacctttttagcatccactctaatgatctcaaccattgtaaatttatacatgaagagccattcaaaaagtacctggtttggaatcatgggctgtcatttttagagcagattccaatttttatattactgtcataaactcttattgtaaacaaagtggcccaaaaccaatcacatggaaaaggatttcagctacatactagacacttacagggctatattattgaaatttacttcataaaccataagaagcttttaatgttggtattaaataaaattccattagctatcaagacatattttggcaatgtcacttgattgtattttatagcattcaaaatgtcttcttatgatttttttttcacatagctccatttattatcattgaacaagctctttgagccacattaaaatgatacggagttcgttttcagttacctaatggaggagcttcttatcttggattataaaatagccattatcttcttcacattttttgcatggcctctccccacctccttctaccagagaagtgtccaggtatcctgcagtcaggttgaaccatgagaaaagtagaactttatagtggaggaaccaggaagaatgaagagaggacatcagctcttcttaaaaaatgatcatagataaccatgtaccagacactgtccagaacactgtacatgtgttaagaatcatttaagcatcataacaaccctttttgatagttagtaacattatcccaattttacagaggaggaaactgaggcttgtgcgtggtggagctaagatttgaccccaggtatgctggttactgaacctacattctatcaacagtgaaatattgcctcccactgagttatttttaatttctttaaatcaaaaagaagagatggttaaggaaataaacacataaacactttcattttaaccaatgtttctcaaaatatacttcactttcatactacttacatcagaatctcatgggaggcttcttaaaactagagattcctgtgcctacccagaccttttcagaccaaaccactgaggagaacaaggtctgggaatcttcttccttaacaagcatcccacataattcttatacataataaagtatgtttatcactgatcttgataaatgttaattgggtaaacaaaagcaaatctacaattaccatgcaggaatacagacagactgtcagtctgtcagaaattatttagcatttatcaataattatcataaatctcctgtcctatcagagatgatgggacaaatcgctgaaggcaaagttggggccagcttgaagcaaagctttgtgtggtccctttatttcctgcttcctcaacttcattctttaatcttacaatcttaagtgctttgaggcagggcactgtactattgcaaagttgagctgaaggtgcaaacaaatgaagtaggcttttggagaatgcagaggtgaaatgacaatagaaaataaatagctatgggcaaatgacacccttgaaagcacatcatttcctgtactttacacataaattcaatcgagtaatgtcattagcagttttggaatctatttgaaaattagacaaatctaggtttgtacatgtgcttctgtgtagaacagaagggactagatgatcttcatgtgactattttttttttccttaaaactttgcctctttctgacaagctgaaatattttaaattctaagaggcaccctttggattaaaagacttttatttttagagataggttatttcttttcttactaattttacttgtttttttaccttaaaattaattttagaatgacctatatgaatagttatcaccattagtgacaatcatatgcaatgagtggaaattttggttttgaatatgtatgcattaaaataattcaacttacaaaagataaaatactgctaattgttcacatcataataggatgtgaccaaaataaataatattttgatcatattattatattttgatcatattgttcttttagaaatagggaaagtcctcaaaggaatgaaactttttaatttattattaacactcagacctgcatttgaaattctttagctttacctttttttttcctgtgataattgacatcattgtttgatctcctgaagtaggaataaatttccacccatgttgaaatcctgatgagtttattctggagtaggagattatcagatcattgtatcattactaaaaatcacagtcccccacattggtattatctccttaaattatagtctcagtgccaagggtggattgttttgtggattaagttgtttttcaaatataggacaaagttatagactagttctaaaatttagttttgtaattaggaatgttgggaaatattacctgtgtctaatgaatgaaggcattttgcaactggaattcacattttaggggaactgttactgatgcatatgaaggagactttcaaacctttttgttcatatattaaactacctgaatatatgtctataaagatctaaaaactcaacctgggtgaaaattaagaaacaatatgttttggtctgaagtcctaagtgggattggctgaaatgctaaaaggttatctgtccagtagtggacctggtccctccagcccaaatccctgggatagaggcataggaaagcccaccttgacaaacccagggctccccaaaagctgaaaatctgacagacttttaaacaacccccaaagaattatcattccaacaatatcttagtgagctttttacatctgagaaagcatggtgtatatttagttaaataacacctgttgtaggaatgctttgggctttgctgctttcaaaaatagtggttatttcatctgaaattctacttctagggcacaactactgaaacagaaataagaaagagacggtccagttcttatcatgtttccatggatttattggaagatcctacatcaaggcaaagagcaatgagtatagccagtattttgaccaacaccatggaaggtatgttaaaagtcctgcgtcacagttacttggtgctttggtaatgatgaaaaaacacttcataaatttcaataaaatacttcctgacttgatattgtatcattattacacattttactaaataacagtaaaatccgtgcataactcatggattctattatcttccacagattttttttttttatatttagcctccagaaagctgctgcaaatgtaaggtatattttgaacaccactttcatacattaaattctaaacattgaaacttgtgtgcatgacgttgaaaagagtgtaatgataaatgcttatacttatgatgatgctaagccatttggattatattaactgcttgagacacaagttataaaatcctatgacttaaccagaaatataaattaaaaatgtgaattagggtttgatattaacttccttgaagcaaagtgtttaaaattttgtagtcctacttttgcctttctctgaccagattcttacaatatatcagctttctctttagttgcagattttatctgaatagttaacataatgtgtagcagtctggatctcagaatgccaaaataaagactttggggacagcttaatctgtgatcaatttctggctctgccatatgttaaatgtgttaatttgtgactttgaatttcagtctcctcatcagtaaaatgtggatgatgatgtttaggcataaggttgttgaatggattaaataagccttcttagataaaacactgatgtatttggcatgcagaagacagttaataaatattatcaatattagttgttttgttgttgttatttttgttaattcacatgtttttgcctttccatactgtaagtgaattcaaacaactgtcaacttcaactacttggaaaatattttcatgtaaaatgtattctatcccccttccttgccctcctattccctcctctccctatctctttacaaaccttctcccttgtaccccttcccaggtatgtgtgtgagtgtgagtgtgtgtagatgtgtcaagggagaagagaaaaggagaatgaaagcaaaagagagcaagcatacacgtccctttcttattgataattagattttctcttgagattggatagattcctggaataattcttttcctgtctgtatgcaaagatcccataatattattaataccaatacgaaaagcctgaaaatcacagccagaaaaaattcacagtgtagacgactgtgtacatcacagacaagtcagtattacaaaacccaattttcatagtgtcctatttcagtatcctaatgcaattcactgatttcaattgaatattaaactctagtacgttcttccccaacctcgcctgcgttagcttgcactccctcttccccccagctgccagtagcttgctcctccctgtccctccaggtaaatcttttgaagattgtctggccttccgctccttgccatagcaaaaccactgagaggaagctgccagtggttctgctaccgatgtcagcagcatgtctgctccctaaagcaggaagtagagaaggagacagggtaagtctaaatcaacagtcatgctttgcacttctgatagcattaagtttgagctaaataagacattacttaaaaaacctcaaatatccacaagattggacttgccaactaattaagatttggagttcaaaataaatgcacccacacctttctccatggaactatgtgacatggggttgcttaggatggaaaggatgttctaggaataagtgcaatctaggaagctgaagactgagagtgttttcgttttattatctgcagagcttttgacttgtgtatttgtgagaaataatggccaagtttttattctgtttttaatcagttatctagaatgaaaactgacttttctttattcaattgtatgtagacacattgagtgtgacatttgtcaaggttggttgttagcaatatcacatacatgcatactcaagcagacttaagatagtcctttttttttttttttttttttttggtttctgataatggtgcaaagttttcctggttgacataatctcttttcttggggatcctttcttctatgtctgattattgtttatttcacctttcctttttatgaaccaggcttgttgatccggttggcaatttttgttctccttctttttaactacagccaagtctccgttgtccagggtaatggatagcctcatgcttaatgaagcagtagtggataagcaaaaatgaaccatttgcgtttcaaatttttaaaagtgcaaaatcacatagaaatgttttctggtcacacctttgtgaaggatggtgggagggtgagttagaagcgcctgaagaatcaaggtgagccagcaaaagacacagatttactgtaagtgattctatgtggataacccatgcaggaataatggagatgtggctgcagttctctcctgaatgctttgctttgttttaaagtgtgagattccccccttttttttggaatgaataattgaatgattttattttagaacttaaacaactttgctccggttattcctactgtcaagatgagccacacctgctgaatttcattttttaaacttttctccagtttattttttttctatccagttcctgtttgcttctcgttatttgctaaatgacagctggcatggaaagaaaaaaccatattatggcagtatacagacaaaattaaattttgtagtttcttttttgttatttactgtaaataataatacctcctttttaccttcaatgtaaatataaggttatttgggagtttagaagtatttacaaaataactagtgcataataactatatttttctctgaattataaatcaaatattatttattgtttacaaagttatgtattaagggaaatggaaacaaactgggcacttgaagaatttcaatatccaaagagacaattgacaaatctatttttagtggaaattttaaaaacaataagcaataaattaatttacttaggaaaatagtattatagaattaattagtggcaaatgttgattagtagaagaaacattatcatctgtggttgtgattgtccttttatatgctggttccacctttacaaggtttagttcatagcaaactgtgccagatatggacagatgttccagttgccacaatagtattaaagtgactgactagagtcaactatgccatggatttaaaaagaagaaaccttccctctatttcagtgctaagaggtggtggccacattttggcagaacaggtaatagggtgtacagcaatgatattgacagaaaacaacaaattctgcatatttttccactcataagttgatgaagagattatcttgccaaaggaagatggtagacagtttttcacttcctaattccccaaatttctttgccaatattcaccaaatttcagtattttgggtgtgaccttaaagatctgtgcatttctgtcttctctcccaatgtttggttgaaattcttcttgacatcattgtacattttccttgaataaatgcattttaatataaaattttatgtcatgtttgatatgagagttatatatttaaatacatttaaataaatgtttaccatgaaaatgtatgaattatatgtatgtttcacctaaaatcctttgtatttttccagtaataaatgagttccactttgtgaaatgttgatttgtaacaacagtgaggactccagttccttaggctggggtattttctcttcttttatgccctctagttaaatgagaaatgtagagagatggaactttgttgtgtctaatatgcaagcctataatctaataaaatttaatttgagacttttaaactgagattggtgacactgacaaaattatctaattagaagatcaccaaaacatatctaatccaagaaactgacattcagtgtgactgattaaggttcttaggacatctcctgagatatctctgataacatatatacttcttgctctacctggaacatggatgagctttaagtgtatgcaatgcaagttctacccattagtttctagcagccttgaagataagtatcagacagtttagtgttgccaatagaatcttggaagctatgtttagccaggatacatttggaaagcttactagcctttctgtactgatcctttctatgacagcaaacccattgtaaaattttccctgttcctccagcagattaacccataatatcttttaacaactttagattttttaaattccttttaatttaaaccaaatctgcttaatagaaagtaagcagttttcatgaggattctaactttttttcttccagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtaagcctcactgagagtttctcttcctcttgaaagagtttataattgccttagtgaattttacatattgctctcaaattaaatatcaactaattggccatgtatatcttgacatcaaatgtttagcatcccttttaaataacaaaaaaatgttgctaccatagtgcaaaagagtcaaagaatttatgtacaatttgatttagaattgaatttaaattgcttatttattagaagatgattctgaattgtcctccaaggacattgatctatagcaaaattctgacatatttttaagaaccttagaataggttctttaggacatgtctgtgtttactaaacaaatgataaaatatgcccaagtcagataattttgaaatatcacttgtaagtacttgaagatggactatgtagggaggggacatcatctgggggattattattttttgtttttgtttcttctacagtcttagcaatattaaatttagaaattatttatttaattttgttaaaatatatattagcatttactggatacatattgatattataatatagtatactatagtgatagattttaaagtggtcttatacagagagacaaaatgaagaaaatcacgcaaaaaagtatatttatacaagtataaaatgcttattaagttcccagcttgataaatgaacatataagcaggttatatagagattgtaaataatacggtctaaagtataatatacaatatttttaggctctgaagtaaccactatattttcgaattatatttcaggtaggacttaactgaattaaatgataaagttggcatatgttggcctcttattttgtatcagatattggactaaatggtttacattaaatctctctctctagattcccacaaaaccagtcttaagatatttactataaattgtctgtatttcgcagttgggaaactgtatccatgagaaattaacagagaaatggaaggtctgaatgctgtatcagactctgaaagctatttcagagactatcataagctatgggcaaagatcacagacgcttagagtaggaaaggataataattttacctagttcaaatttagagctatgtaagaatttcttcaacattatttctaaaaaaaagcagggtggtggggtggcaattgaaacaagaaagcctttggaggtaatatattgtgatccaaattgaatgagcttaagcaaaaataaagggaattcattacctcactttactgaagcccatttggaactagttctaggttctaacctggctagattcaggttctcagccagcatcattgggaatcagcttcttgtctctcttcttcccaatatgtgaccttcctctctggctgggcattttcctactattgccaagattctatcaacttttattctactaatgtagctccatgtacagaaagtttgtgcctcctaactagtagctttaattaaggcccacaaattgaatatcattgacctggttggagttacatggccatttctaaaccagtcagttaccctagctcttggttttaatgccatattcgcctaaatcagaatcatatcttatcactggaggcagagtaaatgagtgaaattatgaagacaataattaggataccattaccagaaggaggatggattctagaaagaaataaacaataaatagacaactcaagctggggctggtcttacgtgttaaggaatgtaggatctgtatctaaggtgaattatggaatagtagaaaatccataggcaggatacacattcttctcaaatgcctataggatattccctaggataagttataggttaggtcataaaacacacatcaataaacttaaaaaaattaaaataatacagaatatttttctgataaaaaataaaatgaaattagaaaccaataacaagtcaatgtggaaaatcacaaatatttggaaattaaacaacttgctcttaaataaccaatgagtcaataagatgtcatgagagaaattagaaaatactttaggatgactgaaaatcaaaacaaaccaaactgaaattaatgagggcagctaaaacaatatttaaagacaaatttatactctaaatgtctatagtaaaataggagatttcccaaattgctaatttaagctcctttttaataaactagataaagaatagcaaattaaacctgaagttaacagaaaagacaaatattaaagaagagaaaaacaacagagaaaaatcaataaaaccaaaagttatttctttgaaaaaatcaacacaattgacaaatctttagttgggctaaccaagaaaaaaagaggaaagacactaattattagaagtaggaatgaagagaggatattaccatggatcttttagaaagaaaaagggagcataagaaataaaataaaattaaacgccagcaaactagataacctatgtgaaattgaaaaattcctagggagtaacaagcgcctgaaactgactcaagaagtaatagactatctcaatatacttataataagtaaacattgaattcacaattaaaaaaaagaaaacttcctgccaaaaaaagcctgagcccagatagtatcactggtgaattttgccaaatgttcaatggagcgttaacatcaatccttaacaaactgttccaacacatagaagagaagggaatacttctcacctcactttctgaagctagaattaccctgatattaatgccagacaaaaataatgcaagaaaagaacacagacacaaatatagaccagccatatcccatatgaacataggcacaacaatcctcaacaaaatactagaagccaaatcacataacatatttagatttttagattatgtactctgaataagtgaaatttatcccagtgatgcagggctggaccagcataaaaaattaatgtaatatatcatattaattttaaaaaactatacaatcatctctatagatgctgtaatcacatggaaaaagccaaagtgtttcatgataaaaacactcagcaaacttgaaatagaaaagaacttcagcctgataaacaccatctcaaaagaccccacacctatcatcattcttaatagttactttagatgcttttatcttcaggtcaagaagaaggcaaggatatttgctcttgctacttcttttcaatattgtactgaaagttctaaccagggaaataagacaagaaaaagaaattaatggcatctagattggaacacaagaagtaaattctattaacgaatacataatcttgtatttagaaaatcctataaaatacacacacacacacacacagctgttagaactaataaataagattgcagaatacaagatcaatatacaaaactcaattatatttctaaacactactaatgaacaatcagaaaataaaattaggaaaattctatttataacaacatgaaaaataataaaatacttaggaataaatttaataaaataagtgtgagatttgtacactcaaagctataaaatattgttgaaagaaattaaagaactactaaataaatgaaagcacattttatattcatagattagaggaaaatattgttaagatggcaatactcaccaaattaatctacaaatttaacgctattcatatcaaaatcccagctacctattttgcagaaagtgataaattgactgtaaattttatatgaaaatgcaagagactatatgccacacaatcttaactagaaaaaaaataaagttggagaactcaaacttccaaattttaaaacttactacaaagcaaaagtaagcaagatagtttggtactggcataaggatagctatatacatcaatggaataaaattgaaattccaacagtaagtcttcatatttatgttaaattaattttcaacaagaccactagacaattttattaaggaaagaagccttttcaacaaatggtgcttggacaagtgaatatccacatatgaaagaatggaattgaacccttacttaataacatatataaaaattaagataggtcataggcctaaaggtagagctaaaactatgaactgttagaaggaaatttagaagtaaatcttcatgacctattattaagcaatgatttcttagatatgataccaaaagcacacacaatagcaataagaaaaaaaaggttcattggactttatcaaaattagaaactttcatactgcaaacaatatcatcaagtaaaaagacaacttaaagaatggaagaagacatttgcaacccagatatctgatcatgatttgtatctaatatatgtaaaggattattataactccacaacaaataaaatagataactcactaaaaatgatcaaaatatttgaatagacatattgaaaaaggagttagacaaaaggccaataagcacataaaagatggtcagcgtcactggctaattttaggggaaaggcaaatcaaaactaaaatgagataccacttcacacacactaagatggctataatcagaaagaaagccaataccattttttatcaaggatgtggaaaaattagaatggttatgctttactttgagaatataaaatgatgcagtcactttggataataatttagctgttccccaaaaagtttggggtagagttaccacatgacctggcagttttactaatttcttcggatatatatatctaagagaattaaaaacatatttcaacatgaatgcttatagaatattattcataatactaaaaattaaaaacaattcaaatgtctatcacttgatgaatggataaacaatatccatccaataaatgttattcatccataaaaaagaatgaagtattgctacaatctacaacatgaataaaccttgaatatattatattaagtgaacaaagccagtcacaaaatttacatatcatattgctacatttatatgaaatgttcataacaggcaaatccatagagacagaaaggagatgagtggttgccaggcagtagggattagggtaaatgggagcaacaactaactaatgggtatgcagcttctttttaaggcgatgaaaatgttctaaaattcaataatggtgatgattttacaagtctgtgaatatattacaagccactgaattgcatactaatatttcatagtatgcatgtatcactattttttcttccaatcacctattgatggatgttcaagttgattccagatactttacccccatgttgcagtaaatgttcttgtatatatcacctttcataatgctgtattaacttctagaagatagatttccaggattgaagtattcccaatattaaacatattcatgtcacataaaaattagatgatatgcaacgaaatagggaaatgtgactcatagttaatagattaaagcggtacatagaaatagatctggacaggaacaaattgttagaataaacaaaaaaggactttggaggcactattgtaaatatgttgagcaatttaaaggaaaaaaaggggtcataaggaataaacagagaacatgagcagaaaaaaatgaaaactgtaaaaaggaataactcagacattttataattgaaaagtacacattctaatatgaaaaattcactggatgtgtttaaaacaaaattgcagatgtcagaagaaatagttgttaacttggagcacattaatagaaattatccaatttgaacactagagaattaaaaggaaaaatgaactgagtctaactataatatgggataatatcaagtagtctaaatatttatataatgggagaatcagaaggagaaaatagaaagaatggaacagagcaaatatttgaagaaacaatggccaaaaatttcccaaagttggtgaaaaatagtaacttacagaccttaatgttaccttataaaatgttagtgaacatcaacaggataaatataaagtaagccatacttaggcacatcttagtcatgctgctgaaaacaacaataacaaaaagctttggaaacaaccggaagaaaaatatttactatatacatgagactaatgtcttaccagaaataattcaggccagaagaaagtggccagataaaataaagaaataaaagcccattattctacagaaagtgaaactatccatcagaaattaaaatgaaataaagaattttaagataaacaaaaacctaaaaaagctgttaccaccagaactacactacaagaaatgttaatggaagttctttaggccgaataggaaaaatatcagatggaaatttgtttctgggaatgatggtcactagatatgaataaatgtgggtaaatacgaaagactatctttttttccttaatttttttcacatttattttaggttcaggggtatgtagacagctttgttgcataaataaattgtaaatcacaggggtttggcatacagattatttcatcatccaggtaataagcatagtactctatagatagttcttcaattctcatcctccttccaccctccaccctcaagtgtctgttgttcccttctttgtgttcctgtgaactcaatgtttagttcccacttataagtgagaacatgcagtatttggttttctgttgctgtggtaaattttttaaaaagacaactgattgtttaaagtggggatttataacataggtaagtgtaaaattatgaaaagatttgaacttttttttttttttttgagacggagtctcgctctctcgcccaggctggagtgcagtggtgcgatctcggctcactgcaagctccgcctccccggttcacgttgttctgctgccttagcctcccgagtagctgggactacatgcaccagccaccaagcctggctaattttgttgcatttttagtagagacggggtttcatcgtgttagccaggatggtcttgatctcctgacctcatgatccgcccgtctcggcctcccaaagtgctgggattacaggcatgagccaccacgccgggccagaacattttatctaaagtgttacaatattaattttacatagaaaattacaagaaagtatccctcatgaacacagaagcaaaaaatcattagcaaaatattatcaataaaatctagcaatagagaaaaaagtaataaatacttcttaaacatgtggggattatctcagaaaagtaagattcatttaacatttgaaaagtgatcaattaattggccatattacctctaaaagaataaaggaaagcctaagatcatctcaatagatgcagaaaaggatctgacaaaactcaacagtcatttgtgagaaaaactgtcagtaaactaggaatagaaagtagctatctcaagttgttaaggacgtttcagaaaaccctacatctagccgggcgtggaggctcacgcctgtaatcccagcactttgtgaggctgaggcagtggatcacttgaggtcaggagttcgagaccagcctggccaacatagtgaaaccccgtctgtactaaaaatagaaaaaattagctgagcgtggtggcaggcgcctgcaataccagctaacagggaggctgaggcaggagaattgcttgaacccgggaggcggaggttgcagtgagccgagattaagccactgcactccacacttcagcctgggcaacaagagtgaaactctgtctcaaaaaaaaaaaaagaaaaagaaagaaaaccctacacctaatattttacttatcagtgaaatgttgaggtagtgaattcttgccccttagaaaaattgagtgctttccccttaacatagacaaatatgtctatttttaccatttttatttgaccttgcactggaagttttatcaactgaactaaaggaaaaaaaaacaaaacaaataaaatgcataaatattgaaatgtggtaaattatctatttcatcaacatgatcatattgtagacaatcctaataaatctttaaaactgattagaaataaacgcgatattaaatattttatttaccataatatcaaaaaacacgaagaatatagtaataaatttaacaaatcatttcgagacacctattactaaaaacctcaaaacatggctgagaataattacagaagatttaaacaaatggaaatatatatgccatgttcatgcaatggaagattcaatattattaagaaattaattctacccaaatgtatctatcaattcaacccaatcccagtaaaaatgtcacagattttctttgtagaaattgatgaactgactttctatgcaaatacacagtgcttaaaattgccaaaacaatactgataaaagaataatgaaaacattaacctacctgacttcaaggcttgttataaagctgtactaatcaagaatctacagtattggcataaacaaagatatcaatggaacataatgctgagttcataaatagatccaaactacatgtcaatggattttttgaaataaacactacagaaaggaaatagaagaaaggaagtgttttcaacaaaatatcaggaacacataaataaatgtatggaaaataaatgaacctccacttcgatcttatgccaagtgccagaatcaataaggattgtagactaagcagaaaagctaaatcaataaaacatctgaaataaaacacaggagaacatctttgagaccttggggtaggcaaaaatttattggaaaacagacaaaaagtgctatctattatttaaatgtccatgaaatttcaaacaatggagacctactgaacaaaaaagaaaagtctagtaatatacacatgggtgaatttcaatttgaagtattcaagtaatatttggaaaagtcactaagactgtcaggtctctgaggtaaccacctaggtagagagagattccatttactgagttagcaaatatcagaagagaagcaaaactgtggagaaaaagcagtaagatgaattttggacctgttgaatttgagatgcctgggagatagtctttcaaatgtaggtattgaataggcaggtgggtatgtatttctagagactaggaggtatgcttgaacagaaaaatagattttgaaatatgaactattataaaaatgtaacttattataaaaggaaaactaaagtaagaggggtctagacagagagagttctgaataaatccagtatcaaatgatttgttagaggaagaaaatcaggtgagtagcatccagggaggtgtggatcacagaagctaagggcagaaaatatttcaatgaacaaggaacagaaaacaatgcctgaaacttctaaaagggcaagcaagcaagataattgtttaaaaaatttcgtttggatttattggtgatgtcggtcttgttggtgaagtttgttaaagccatttggtgggttgtggagtaaggtgaagaaatggaactggcaagtgtagacaagtatgcaatcttcgaaagaaatctggccataaggaaaggatagatggtggcacctggaaggggaaatagagtgcaaggagagtttctcttatgacagtggtgatatatatattttttgtttgtttgtttgttttttgagacggagtcttgctctttcgcccaggctggagtgcagtggcgctatctcggctcactgcaagctccgactcccgggttcatgccattctcctgcctcagcctcccgagtagctaagactacaggcgcccgccaccgcgcccggctaattttttgtatttttagtagagaagaggtttcaccgtgttagccaggatagtctcgatctcctgacctcgtgatctgcccacctcggcctcccaaagtgctgggattacaggcgtgagccaccgcgcccggcccacagtggtgatatttttaagggagagaaggactcggtaactgttactttctataaagaaacgggaagtagccagtagtagaaatgttggttaattgagaatgaattccttgcaaagtccaaaggaaaacacataacacaatttgagagattagctcaaaacaggggcacttctttcattttaacaagaagagaaaggcaaagcacagttacggatgtaagtagtctagtagataaagggaaacaaagttcaggctggctgactttcatcacctctaagacgctgaagtactgagactgtgcttctcagatgggaaggcataagcaaaaatggctgaggttatgtgcagaagagaaagtttgaaatagttttagataatagaaatggagaaaggaaaatactgcttccctttcgccaacaaaaggaaatttttttaagagttcttactatctgtagagctagccgtgagcatgtttattacagcttacatgacatcttagcaccagtctcatctgcagggccaagggaggggaccaatccatttggcttggtgatggaagcccgcactgctaggtaatcatttggtaagtttttggagggctagaaagatcagagacagagccaaacagttgatcacaatgagtcagttgcacctttcatatgaaaataatattaattttattgacttaatccgtgtactctttatcatttgataaacattatatatagtgaacaattattgatttgaatgcaaagcatttgtagatactaagttgttggacctaaaccaattttttaaaatcagaatttaatttatatttgttgggagtaaattaagttgctcaataattattcgtgtttcaagagtatttgctcatataatgaactacacttctcatttaggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctggtaaattaactgggagtgttcataaaatgtactttgtaattaattagtcttcattctcatctagtaaaaatggcaagatttcccatcattataatattatttgaatacacttctaaaacaaattggattgccataccaccaaatggtagtttcttcttcatcatagctttaataaagttcacttaaatgaatagtctacacttctcttcttagttattgaatggaaggctaatagagaggaggaaacagggagtcacagataaactcgaatcacaattaaacaacaccatagtcaactctcagttatctgaggtttgcataactgcgtacaaagcttccttgggacctaggatgagctcccctttctgccaggaactaaagaattatggaattgttcattgctcaccttgtccccgtagaggaaagagttaagacaggggatagtgtacaaaggagaaggataagcaaacagagctccccatatgactgctgccacatcagaaaatcaccaaatcactctttgaaagagttaactgtactatattttgttaattttaaagaaagtatctttctttgatcttttataaaaactattagatcttaaaattcagagataaaatatcacttgacacatttccagtgaaagtttgatatgttttgttatactattactttgagttggctctaagttagtgatttattttcaaataacagaggctgtacacggttactaaggacacgttcctatagatgatttaccttagtagtgattaggctgaagactttttcatgaaatctgtttacaatttccctttctgctttcaatgttcaaatttgagttgtaatccttagaactatatttccttccctaatcctcaaagatagttatgaatctaatttgaatctagaaggatgcaaaaaacagaacaaaaatttaaaatgataaaacaagtaatatgggcaagaacttaaaaaaatatatttagtaaaccttcatgatagtgtgatgcagttaagggaaataggaagcatagtatcactagaatcttacttagtgtgtcaggctcttttgcataaattattctctggaataaattaaatactttggtgcatgtatttactcctttgggtcactttgatgccattaaataatgcactactttcagcctgacatttactgaagcatcagaaataaaatgctgctgctctttaaccataaatggtacttcagtgaactctaaagctaatacaaccaatatgtcaaacacaatgagaaagacatttacacactacactgaattaagtctatgaagatataaaggttaaaaagaagcctagcgttttacttaagtttaagtatttttgtatttgaatataatatatgtttaaaatatagcctaaagttacagcaagctaaagatatagctagattaaaacaatctaaagacaaagaaattagttcatttctgcttccactttatgtaatttaagtgttgatattattctcacctgtgcatttcagcatatttaaagtacactgaaaactatatctgctttggccttttaaaaataatgagagttcctacttctctgaaactggatctctgctaattaaccaccattaatctgaaatatcttaattccttaaggagaaacaaaagtgtatattacatatgcttatgtaggatacttgaaaatttggtgtatcttattaaactgccaatttaaaaactgtataatttaattatttcatttacagtatggaccatttcaaaatgaaaaaaagaatgctctatggtagcaagtcactgctatatttgttagtgatcatttgacaaataaataattcatcattctataattgagacagttacctgtacatttgccctgttaataaaattacagatttttcccttcctgtgtccatgtgactaacctgcacattgtgcacatgtaccctaaaacttaaagtataataataataaaataaaataaaaataaaaaataaaaaaataaaaataaaataaaattgcagatttttttagaaatgcagagcattaacactgttcttgcttttatttccagctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggtatgtagcaaaaacattttcctcattttcattaaaagataatgtaatcattaaaaagtgtgttcaactgaagaatattttgtattttttaaatcaaggccacttcctattgtctattactcatgactgtaagagccatgtatagtttagaccattgtaatccacacaaacccttaaactaccttttgaaccaaagttattctttctttcattatccttcttgctacaaggagagaaacttttctgttatttatctttcagttcttgtactagagcatggaagtgttacttagaacactcattttatttataagtactagcaataacacctgaaaacgtttcagatttggttttctacaaatttaaaaactagcaacaatctcagtttattaagagctcatggggttttcggtgcctagaaactatggtatgagcaagtaacattgtctctaaaaacattaattgtcatttctgcataaaattaaccacccctaacaccatatatatttaggatagttagctcttcttgttgcattgatcccttttaccattatgtagtgtctttctttgtctttttttaatctttgttggtttaaagtctgttttatcaaagactaggattgcaaaccctgctttttttttttctttccatttgcttggtaaatattcctccatccgttttttttgtgcctatgtgtgtctttgcatgtgagatgggtcacagcacaccgatgggtcttgactctatccaatttgccagtctgtgtcttttaattggggcatttagcccatttacatttaaggttaatattgttatgtgtgaatttgaccctgtcattatgatgctagctggctattttgctcattagctgctgcggttttttcataatgttgatggtcttaacaatttggtatgtttttgcagtggctggtactggtttttccttgccatatttagtgcttccttcaggagctcttgtaagtcaggcctggtggtggcaaaatctcttggcatttgcttgtctgtaaatgattttatttctcctttgcttatgaagcttagtttggctggatatgaaattctgggttgaaaattcttttctttaataatgtttaatattggctcccactctcttctggcttgtagggtttctgccgagagatctgctgttagtctggtgggcttccctttgtgggtaacccgacctttctctctggctgccattaacattttttccttcatttctaccttggtgtatctgacaattatgtgtcttggggttgcttttctcaaggagcttctttgtggtgttctctgtatttcctgaatttgaatgttggcctgtcttgctaggttggggaagttctcctggttatcctgaagagtgttttccaacttggttccattctcccagtcactttcaggtacaccaatcaaacttagggttggtcttttcacatagtcccatgtttcttggagactttgttcgttccttttcattcttttttctctaatcttatcttcatgctttacaaatttaactcaacatggattaaagacttaaatgtaagacctaaaaccataaaaaccttagaagaaaacctaggcaataccattcaggacattggcatgggcaaagacttcatgactgaaacaccaaaagcaatggtaacaaaagccaaaattgacaaatgggatctaattaaactaaagagcttctgcacagcaaaagaaactatcatcagagtgaacaggcaacctacagaatgggagaaaatttttgcaatctatccatctaatatccagaatctacaaaaaacttaaacaaatttacaagaaaaacacaaccctatcaaaaagtgggtgaaggatatgaacagacacttctcaaaagaagacatttatgtggccaacaaacatatgaataaaagctcatcatcactggtcattagagaaatgcaaatcaaaaccacaatgagataccacttcacgccagttagaatggcgatcattaaaaagtcaggaaaccacagatgctggagaggatgtggagaaataggaatgcttttacattgttggtgggagtgtaaactagttcaaccattgtggaaaacagtgtggcaattcctcaaggatctagaaccagaaataccatttgacccaggaatcccataactgggtatatacccaaagggttataaatcattctgctataaagatgcatgcacacgtatgcttattgcagcactattcataatagcaaagatttggaaccaacccaaatgcccatcaatgatagactggataaagaaaatgtggcacatatacaccatgaaatactatgcagccataaaaaagagtgagttcatgtcctttgcagggacatggatgaagctggaaaccattattctcggcaaactaacacaggaacagaaaaccaaacactatatgttctcactcataagtgggagttgaacaatgagaacacatggacacagggaggggaacatcacacactgaggcctgtcgaggggtggggggctaggggagggagagcattaggagaaataactaatgtagattacgggttaatggatgcagcaaaccaccatggcaagtgtatatgtatgtaacaaatctgcatgttctacacatgtatcccaaaacttagagtataataataatttaaaaaaattaaccatacccaacactagtgtcctgaatcttgaaggcatggagaagttgggaaggcatgggaagataaatataacaaagtgatataacatgtactcaaatagaattaaaaataggaagtaactaatatgtgtccaaaaatatgaaaacaaagtgccatgtgtcaagtttacaaaatgtaaaccttgctttacaataggaaggttgatcagggaagtctttgtcaaagagtttggacctaaaatatatttaactgagatgtaagatttagcttggtaggaagaaagaccatcccaaacaaggaaacaaggtacccagtgactgagggatacaggacagtagactctgtgagaagtatcaggctcttatgctttaaatatgaagtaattacaccgagttgcttaattagaacccaaaccaatggaatagaaaaatgactaccataacaagtaatttaatgtatatactcttgccaggctcagtggctcacgcctgtaatcgcagcattttgggagactgaagtgggcgtttcacttgaggacagtagttcgcgaccagcctagtcaacatggcaaaaccccatctctactagaaatacaaaaattagccaggcgtgatgatgcacacctgtaatcccagctacttgggaagctgaggcacgagaattgcttgagcctgggaggcagaggtggcagtgacccgagattgtgccattgcattccagcctgggtgaaagagcgagactctgttgcaaaaaaaaaaaaaaaaaagcatatactctttagacatgatttcctctcatataaaggtaacctccaagtccccaaagatagagaaaggggaagggaaaaaggcaaagtattattttatttttattcattgccaaatttcagcctcttcaacattacttttgataattctgatctatttttaaagtaacaagaaacataaacagtgtacaatctagaattataaacagtggcttaaaacaataaacactgattacttcatagtttctgtgggtcaggatttggggaataagttagctgggtggttctggtttaggatcagtcatgaagttgctgtcgagatgttagctgaggttacagttatcttgactggggctggaggatcagcttctaagaaggctcaatctcatgattattggaaggaggtttcagttcctttttggcagttagttgaaggtctcagtttttctctgcaggaccttttccataggactgttgagtgtccttatgatatggcagctggcttcttccagggaaggtgatgtaagagagaaggcaaggagaaaatcctctttatgttctactcttgaaagtcactcttcaccacttctgccatattgtattcattagaagctagtcactaagaagagctcaagctactataatccccaagacaactttaaaatgtttgctttcagaaaagtataagatcacatagaacagaaagtgccatagggttacatagaacagaaacaaagaaaagataatataattatgttatagatttgatttcattttctctgtatgtatatttggtatatgttggaagaagaaaagaaaacgcagagaacagaatcctttatgacaacatgaatgatcagacagcaatggggaattaagaaatataagtttgggaccagattgggtagaatttaatttatgaaaaggctgactgtgcataataaaatgtattttcctttaggcaattcaaagctacagatgattttttatcaggaaagtgacagtgaaccagtgatatttttcagaaatatacgtagcaggagaatgcagaatagatttaaagaggatgaaactcagcccaccacatgttatctattagtttactgaaattaacatatctctctaatgtataaatgtgcagaaaattgaagttgaaaagagaatttcaggaaatatcaagtacttatggttgacatcagtattaatttagattgtgatgtatgcataaaaagatatagtttataaaataatcatttccatctactgggtgtaaatttaatttttgttcttttaagagagaaaaattaaaggttctcctttctttttgactatcagttaaaataacttctttgtcttgtgataacctgggtatgtttctggagtagctaaggtagtcatatatatcatgtttaccactattaaggaaatgtgcttatataacatttgcttaagactgaatgaacttgatatactcactccttactacaattcttccttcctattctcactggaaaaatgggaaaggtgtcccaaagacaaaatggcataacttccttttaacacacatgaactatcagatgtggctccacccaaatagatgtagtagtcacaatggatgggactgccagcctagtctacagacaagacagagctgggaccacaaactactgtttcccagaccaggatttttatgagccattcttagtttccagacacgatggcaagagacccttcattggttgaagataggtgctgcagaaaaagaatgtgactttctgaaaactgatagttctagaagcagagaagacaacttcctctctccctaagtgaaggtgaggcaatagcacacaggagggatgtgaaggttttggcttcctctcacaagttgggaatcaggatggagaaacaattaaaatatgtaatatgtttcaaccttgaattcaaaatggaaattatggtaacatttccattccaagaggctaatttgagacacaagaaagagttgatttcatttactgagctagcacatttgtgaaacaggattcaggatttcagtccctgagtgagcttgctgaactgttttctttcttttttttttttttttttttttttttgagacggagtctcgctctgtcgcccaggctggagtgcagtggcgggatctcggctcactgcaagctccgcctcccgggttcacgccattctcctgcctcagcctcccaagtagctgagactacaggcgcccgccactacacccggccaattttttgtatttttagtagagacggggtttcaccgttttagccgggatggtctcgatctcctgacctcgtgatccgcccgaggcctcccaaagtgctgggattacaggcgtgagccaccgcgcccggcctgaactgttttcttaaattgtcatggatcacaccaaacacctgtgccagctgttatgcgcatacccttcggtaacaaaggaagtccagaaaaagagaataacttgactcacacaaatatttctagggaaataaggtaaataaaaagatagtgttgtggaggataagttggataatagtaagtgataacagctaaactttctcaaaggttcactatgtgccaagaactgtgctgaaagccacatgaattctgtcactgaatcctttcaacaaccttgtaagctgggcactagaaaaacgattacatttattttatagatgaggaaactgaggctcagattggttatgctacttagtaggtaacagaaaatcgattcttacctagcactcgaattctaaaatatgtgctcctctatgtcaagtaatctatagaactaagataaacatgctgatgaaagttagtgtctagtgggtattaataaacgcggtttcaaaactgtgtcaccacgggtagattggctgctttaaaaaaaataaaaacttcaatggatttatgagaaagaaaagtcatatgttccagggatatttatttattttcctgacagtggaatagcattgaattgagttatcaattcataaaagatcagagaaacaattcgaaaattaatagtaaaccctattatactgactaaatatggtagcagttcaaagaaaggaagtatcggtaagagtaaacatggaatactgtttcctcacttattctgcaaacatcacaattaggagaaaagaccttgtagttagactttcaaaaaaaaaaaagcttctctacttattaactgtggtcttggacaagtcatgtaagttgtgcagaagcattttcatctgtaaaatagtaataattcctgccttatagagttgtgagaaataaatcacataaaccttgaaaatgctttgcacaataactggtatttactaagagctcaactaaaaagctggttttacttttattgttattatgatctggtattgatactgctctaggacttggctctaaggcatgtttctgaccaaaagatctcccaatctatcagtaaagctgtgcttgttttttttttttttttaaagaatccaatataatgtgatagggatgtggatgagaaatttaacaggacagaatgagaaatgggagcaggctatataaatgtcacagtaaatgacatttgtaaatagagtatttgtccagtacagaggcaatataattggctcccgccctggggaaggattgatggatgtgtatcaagaaaaatttccaaatagacaaatgacagaactttaatatactttaggaaaataagtctaggaaatagcaccaaaatagataaaataaaaattttacatgcaattttttcttcctttgtctgtttttttaatccaaataataagttcaaaagcaaattacaataaaacataattttattggtaaattccagaggcaaaggagcaggtctggtcttaatgtgattatcaggagtcatagtatagagactgacagattgtcagtacactctcaaaatcaaacgtggtcttcattggatcttacatatttttactttaaaaaaaatcaccattggttagactaacttacaactaattagacaaaggtgctgtaagcctcattagcatgatagaagcatgagaatatagcaagaatgtagaatcctttttattgaagttttacttaaaaattttcctaagattctacttttgtactacagtttgagcatccctaatctgaaaatttgaaatccaaaatgctcggaagttcaaagcttttgagcaccaacctgatactacaagaggaaaattcacatctggcctcctgtaatgaatcgcagttaaaccacagtcaaaatgttgtttcctgaactaaattattaaaaatattgtataatattaccttcaggctatgtgtatgtgtatgaaacttaaatgaattttatgtttacacatgtgtctcattctcaagacttgtcattatgtacatgcaaatattttaaaatctaaaatccaaaacacttctggccccaagcatttcaaataagggatatgcaacttgtatttactttgtgcatttgcccccctttactgctatatctttcttttgttctgtatgttatgtgtgcttaaataatcaggaattcattgatattgtcaatcaaatcctgaaaaaaaattatatgactcagtcttgtacccctgagaatgtctgatttcttcgtaagttgtctttttttttttttccacaatagtgagtttaatgtcatgaatcttttcactcattcatactggtggagcctatttttaaagacccaatttgcagactgattactgtccttattcatggcaatacttcaactccacaatctttaattcaacaataacatcataattattgtataataaccattttatagtatttctcactattgtataattatagtagccataattgtcttaataaaaattgggacttttcatccagcaataaatacgtttttgtctgatttgtccagttatctaggtacaaaaaatggtacaaaggcacaaaaataaaatcatatttaaatatattgggataattgttgattttaggaataaattatcagtgtttccggaaatccaaattacatagtcaaaatagcatctgtattaggccattcttgcattgttacagataaatacctgagactgggtaatttataaagaaaagagatttaattagctcatggttctgcagtgagcttggtgctggcatctgcttggcttctggtgaagtctcaaagagctttcaatcatggcagaaggcaaagtggagcaagcatttcacatgatgaaagcaggaacaagcaagagagaatgtgggcaggaggcactacaaacttttaaataacaacatctcatgagaactcactaacatgaggacagcaccaaggcatgagggatctgaccccatcatccaaacacctaccaccaggccccacgtctagtgctggggattacaattccacatgagacttgggagggggcaaatgtccaaaatatatcagcatcccaaataaaagggttttttttgtacagttgtctatatttatcttttggaactgagcttaatagaaatgtttcatttagcaatgatttcagtattttctgcaatgactaaaaagcaaatagtgataatagtattattttatattgaccaagcatttttatttcattcactttttttcagaatagtgtatcatgaattagcagaaatgcatgttagaataaaataaggtgtcaagaacaatcttagaaaactaatgatggaaagcaattgaagcaatagaatgttttgatcacctgtttttcctgctgtgtttcaggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagaggtaaaaatgtttaaataaggagatattttggtgttatataattctgttgtttaaaattatcaggtgtttttaaattgcgtgtttccttcctgttaagaaaatagaaaatatctgtctagcaatatattttccatggaaaagttggtaataaataaattaatgatagattaaaatatagctagattaacaatatgctgacttatgtttccaatactgacattttgaattcttgacagtattcttgatatgaattttttcagtatttataaataattttaaatttctcaaaatgcctcaatttctccactttcttccttgtaatttgcccacaacagtgttttttgtacgtactggaaaaatatctgatgagagggtagttgcaattctcatcttgctatgttcttagttcttaattcttacgaaatacgtcataaaatagtattgtattttgtttgcacagacatatttactcaaggaagatctgattgggatcttggcttgatatttatgtatagtttatctttcctgaagtcagtcagtttttttgaagagaaggtattgatgaggaatcacactaaaaacatatttaaccctactgagctcagtgttcactgtttaaagaaacaaaaatccttaatacattatagaatgtaaaattctgaatttaccaactcagtaagtcctggtaacttaatgtattctttgatttacaagaagggtatgagcaacagaatatattttttgttttgtttgctattaacctgttgctcaataagtacagagttggaggtaaagagaggaatttaaaaccttgatatttaattgtttatacaaaaatgaagacaagatttccagtaattaaagtttgcactaactaacaaaaataacaaggaaaaacaaagattcgttccttcctcatacgaactgtttggcgaggaagataaaagcttctattcctgatgtcgggaaagaaagaatgacgacatgggggagtgtgggcactgaaaggtaaaatttaagtagcacaacatgatcatgataattaacaatcagccaaaattatgagggaaaatatagttataaaaaaagaacaaagatgggtggatcacgaggtcaggagttcgagaccagcgtggccaacatggtgaaaccctgtctctactagagattcaaaaaaattagccaggcgtggtggtgcgtgcctgtaatcccagctactcgggaggctgaggcaagagaatcgcttgaacccaggaggcagagattgcagtgagccgagatcaccccattgcactccagcctgggcaacaggatgaaactctgtctcaaaaaaaaaaaaaaagaactagctatttcagacactttttctgtatttatttgataaaattactaaagagtatgttattttccattttttcttgtttgtaagttacgtagtattgctgttagtgattaggtagaagtagatgtttaatgggaaattcagacaatctttgaatataggaaggtataaataacagggacataggtatcagtttcacaagaaataactgatgagattcaagggaaaagtaataaaaccttctgtcctggggcaaagaattactttaattggttgaacttaaatttttactaactagattattgtttgaaagttgaataatatcttaaaatcttattaacaaaattttgaacaagtgttgttacaatagttgggttatgctggaagggtggagtggcccaatttcatatacagtgtactgctcttatagaagctgaagtcggcatttataaaatagaattcggtcatttgaattttgatgtatattcccctctcattattttgaaattatgcctaatggtgaatatttccctaatagtaaaaaaagtcaatttttattttcacacatgtttagttttaggctgtcatataaactaagaatgaattatacagtatcaaacgttgaagccattggctagtttaatcttttagctaagtttcagtatcttttgaggaatgtttaacttgacatccagtcttcttaactttaagagattttacagccgtgggttttccaaaagagcgtgtattttgccttaacttaagccattatgtctgaagtaagagggaagtccagtgatgtggggtttagagtagggacatctcttgtttctcttgttatcattaagctttttgatttgttttcccattaagttagctctgagttaaatactctaaaataatatttgtgaattcagtatttcagaattggaggaagagaactgacctgccaggtggaagcagacaggattattttattgcttgagttgtggagtccttccaataccttcccagcatagagactgttacttcagtgttaacattatttggaggggtttttaattctggctttatatcaaactttctagacataaatttataaaataataaatgatgagggttatcgccgtgaaagaggttatgtgtaggttttgatctttcagaattttacctggtagctctacactaaaaaactagagaattaaaacaattattgaagaatttcagacactcgcatttgaaatagcatttcttgcctgccttctagtcatttttgtctggtcatttttctaactgggggacaggattacattgttaaatatcacaaagtagtaagaaacatcatgaggcttattaccaatcctttctaaattaattttttaattaaagaaaaaatgaggcttttttactggaatgtctaaatgaatttttttataaggcagactgagtggactcagaggttttttaggtgttcacagtaagtcctctgcaatgtctttgctaaatttgtatgattcttcagtagttttctgtagattctctagagtaggccatttaaaatcatgtcataatcccctatgctttaattttaatgttatttcgattatattaatgtaattccttttgtgattttgaatgattgttttttcttttagagtatttaataatgtggaagccatgcttgaatgactatttttcgaagtgaaatttagtagtgcgatatggtgaccttcaccgcttaccattcttacttctcacaggagtaaaatcaagctggagccatcaagaatgcagctctggtgttttttaaccagccagaggctcgtgccaccacttttacccaggttacccaagcaagttgtacatctataaatataatcagtttctaaatgacttttgactggcctgcatgttactcagctacgttccttgcccttccattggcagtaaaataaaaacatgcacagctgctattatgctgagtcatacaaagcatggtcaggcaagtctgacaaccctaacttaaaaaaaagtgatttagctgctaattttcttacatagattttaatagaaattttattcaatgaaaagtaaaagtgcatgcctttatggattatttaatttccttttaatgttacagagttttgaacatattaggagcccaaaggagaaatgtaggtgctctttgaaaacttgcaaaaatgctttttatcctctgtctttaaaaaaaagatagcccagttactgtacttaagtcttgacagttttttatttagtgtaatgtttttctgaagggtaatcttcaaattaaagcaatcccttattcatatgcaaacttcccaaaggatgttttaatgtgataataatgtaaatgaataggaatgtctgtgtttcagttgctagcagcatgggtataatatttatctgcttcattttagggaaaatggcactgctttatttaggagttgaccaacagtattttgtatttagaatataatttctttggaaagtctgtttatatttacccttaaaactcttagactgaaagaaaaggaaatcatgtcttttgtataccaaataataataataatagtgataatgagagacttatagatggtatgctccttctaaaaatagatttagagtccatcttcttcattttcttggctcctctgtgctttctctcccctctattttattgagacctgctggaaaactttctcccgaagaatattatttaaatttatcatgatccacaaactcctgtataggaaaagaatcagaaactcttgctcctagggtgtttttaaaatgaagagacttccctatcatgtgacaatagcaataaacgtaacatcattctatgggatccattagtcgaccttcatttcttaatgttgaaatcacagttttatgcacaaatatttaaccaaaatgcctaaacccaatttaatcatttttaagaaatgttaattattttgtcacttagatacagtttcctctccttttgccaataaaactataaaacagcactaatataaaagtgtagttggctatttggaagaagcaataatcatgccattcctggagcattcttttatactttggaacaaaatattccatcactggctctccagattcatgagctataatgcctcatatattggaggaatgggatgtaaaatgggatccaagatgcgtaattgtttacagttaaacacagatgcgcatatacacagggactacagataattactttttcctattatgtattaattcttcagaaaagcatgagatttaggcactttcggataatagcttgtttctcggaaagaggcaagggtagtttccttattctctgagtatcccattttgccaatttcctgtttagaaagatacttgaggcatattatccatcaacgtatctaggggattcagctggagtaaaggtggtagaatagaagctaagaaggaactggttcgtttattttcaatcctcacattatggcaatttttgatttccttgtaaaagtctatgattctccctcaggaaacattgtccacttcctaaaaaaatatactaatttctaatacaggggtttggaaaggggacaaaaatgtgcagggaaggtttgcgtaagcaatggtggaatgggttcaacagacacctgtctatgactttatcctggagaatgtgtagtcctcatgggaaagttttccagtgggatagtgattaagatggaaaaaaatgcccaaaatatctttaatataagaacaaaatgggccaaacacgtgtctttgggtcactggtaatctactgagcagtaggacatcatgacataagagttccttttgccatccgaagaaaaatatttaaaatcctattatttgtggttttaaaaatgttataatgtattcattataaacactaaaatgactttctggataatatagtatactgtgagtaattattttgattttaccatattcttttttagttctcagaaaccaaaattgtcagatatgggatacttgatttaatctgtatttgaagttttctcttttttaagtgccaattttttaattaaattaaattaaaatctctctctcttttcccaaattatatacaatatctactaattatgttttcttcgaaatgtatcttagcttcataatgagaagtgagtgtgcccatgaaaaatttaataggaagttatgttttctcttccattttctgttgtgattcattatttttgaaaataatttactttcatttgctcacatttgctgtctaaagaaaaactattcatctggcacattcatatttagtagtattattaaagcagaaagcataagttggaagtataatatctaaaaatacaaaatgaagtattgtaccttgatgtttattagatcattaagcaaaatatgattctgccctgcttaaatcatttgattataattatccagcatataaaagaatcacagtagattttcaataggaagagtcctataatattaggtatccaccaaaaacattgttcaagtaatatttccacctgaaagtaaatgattgccaatgctttttttcagagcatataaaattggctattcctatttgatctcgttattgtcctggcatccttattttgttaaattttaactaggcaagggctatgctacaaacatcagttagtccgctagtttcctgataaataagtacaggtaattaaaaagtgaacctaaatatccaaattataccaaagggacatatatagaactttttaaactggatctgcttctagccagttcatattttggtcacttactaatgtagtatttcaccataaattatgcctagattggagcatttacaggcactctttatctgaaaattcttaagtgcatgagttgtaacagtttcacatagtgatcatctcactgttctaattggtgactatgtactacagttaggttgatcgtaattatgaccttaaatgaagctgaatatttttatattcctaatttgattaattttattttatgggcttttacacattttaactgctttagtcaacatattttataatattatgacatttgcctagaatgtaattttaagaaagtcatttaattgatgttatcaagaagggttttataaatcagaatttcctgcctatgtttcaagatcattgcttaaagaaaactttttgttcatgtaatattccaatgtgtatttagctttactctacattaaaatatgttaacaactatgaatataacttgaattaatactaaagttcatggttttgaaacatggaaatcaacaatatcataagcactatcttgaacctacaatatttgattacatatctagtctactaaatgttttaaattgataaacattgggtttacttttgaatcatcaaaaagattctttagagaagcttggcagaatggggtgcagaagatctggaatccaaccactgattcgctataacttttcacaagacgataaaactcacatttccttctctaaataaatactggattggctgacattaaggatcaatgtgctgcctagattctttgttatttgtaaatcaagtaccacaagtggaaaagtattcaagtaacacatgtgacagatcctgtgctgctccgcttcagaagacagtggggaaggataagattgcattccttaaagaggccccattcatcactggagctatagatccttgtatacagagtgaaaagagggaaaacactgtcaaaatgatttagtaatagttttcctgactccacagttaaactacagttcacctcatacactcacattaggtccgaataattggcagactggttttaagacaatactcctagttctaagagttgttcgtcattgcccacacaattcagaatcttaaaagatttgtgttactctgcaattaagagaaaatattgtgtgtattcttttgaatgtgaaagtaaatatcagataggaagtgttagtagttagtggttggataaaagagtcctcgcactggtcattcattcatttacccaaaatttattaaacacaaacattggtgctagagatacaagggtgacgaaaacatgatctttcctccaaaaaattaagtctgatgagatgcattttctagaaacacaaaatacttttgaactgaaccttggaaaaagtaaaaacttgacctttcaatagataaatatttggctttaggaaaaaggtatcttaattctacatcagaactaaggtagtgcactaaaatgaaagggagcaatgttaattcttctacttttaatgtgatttaaataagagaaaatacaggaatgtcttttataatttgaaattccagagaaaatgaataaaaaagcaattaaaaaaaacacctcaacatgcttctcattttcagccaagtacagcaaactctgtttgatattctctgattttaaccttggatcaaactatttggcaaattgctaatttgaacaggctattgaaaacagacagtgtatctagcaattcattcattcattcaatacttaatgaacagcattttggcaattcaaagcctgttccttgtattgaactatattggtgtattatccatatggcctagagtacatgtgtattattcatattatccatattggtgtattatccatagggcctagagtacgtggcttctgcccccaaatgtcttacaagcatgttaaagtacccaaaatccatccaactgaaataatttgacccatacattatcaaggttcaagatgtgtgataagccatgaaaaggttggtgaatagcctagggtgcatatggtgagtgaagatggatggaagaaattaggagtccagatttattggggagactttatgaaagcaaggaatcatgattgttgcaaaatagacagtgaagaaagaaatggtgtggtggtgaaagtcttcccaaagtcatttaaagtattggcaacccagatacttggcagcaggagtgataattgatgttacaggaatcctatgtaaacttttgcacattttaatgaagattttctataatatgctgtctggtgacttctcccaatcactgtcaagggctagacttcattattttaagatgtctttcattcatttattcactcactcattcttttttttaagcaaaatttattaagacctgtaacatacttagcactgatgtaagctctgatacagtcattcagtaatgtcagcctgagcatttgcagaaccctgaatatggatcctcatttttcccttggggtcacccgaagtcttaccccgttctgtgtttacagtgtgaaccttattcttataaggaatattttttgtttcttattgtggattgtctgtacttccatcagatgtgactcagcttcagtttttctgattacctgtgttattttccaacatgttgcaagtgataagatcagtattatcaggtccctaatagcataacaggttcgtgtgaattagatatattaaaatgagaagatttttaagtcattttttagtcaattacgtgtatagaaacatagttatacttatctcagaaagattccaattcaaaggataggaaattagaaaaacatgtaatgtttcttctgagaacaaatatattcaaattttaatgacaaagatattaggattatttttctagtaatttgatatacctgataaattaaaataagaaatcatcaaccttcaagcaccttataatatataatctttgatttaacttttttggacaaattaaaaaataattctctaactcaaagcaggcagaagtatctaatatttatgcttccatcattagtatttttaaagtacaagagacaaaagcatttttaatctcatgtatatcatacatcaatattttttaagtcatcagcatcactatgctaaggaaatattttatataaagaaaattattttcataaaataaagaaaccatctttctagagaaaatccacataatcctatgcccaaatataaatataacatttacagtactgactacagcatccatcttattctagtttaacatacttcagtgtgcattttatcatgtgcttacctcatcctttttataaagtatctcttatcagctcaaccatttcctaatgttatgcactaatggtaataacattgtaacaggtccgagaatgctgtggcccaaactactctgattcctgtgtggcactcacacctggccctgcatcatttctagagcattttaagaccaggttaaagactgggatcccttcatgtatgctgatcccaagaatggaccgacctggctaaagtttattgcatttcaagatataaaacaacttctattatatttttcttttggtgatatttgatttaacctaaaggaaaacaaaagaacaactaaatattcatttctgcctgtactaacagggcaggtaagagtgccagagtaacaagtagtttccaaatgcacaatgaaacacaggagtgcattggccaaagagaatgcaaaatatcagctcttgctatatagctaacaatgtgctgctctttttggaattagaaaattatagaatatatttaataacaatctggtatatgttttcatgtcaatgaaaagtggataaatttagatggttgctgtttatgtgcatttgatcaaatcttttctgaatttgacatgaaaatacacttgtgcagctttcattggttgggtcacaattttagaataaaacaaactatttgaaaaccatttgcaaactaatgtacaaaagcaagatcgcagatgattatatgactctggcagcttacataagctttctgcaggattttctttcagaatctctatacataggctcaaacagaagttatttccgttgttagcaccatattttaaagaaaaaaaatactatggtgttgtatctaatcttgtgacccctgacctttaccaaagcggattggcattatgtttaagttcttaaattacagatcaagaaaatgcatacagaagatggggggggggcacacctaattaatttttatatttagattaaagaaaataattaaatgtgtttttttgtgggattgattttcagaagctaaatgcaactagttcatctgaaggcagcacggttgatattggagctcccgccgagggagaacagcctgaggttgaacctgaggaatcccttgaacctgaagcctgttttacagaaggtaagcaaaacaataacatatgtggtcttgagtatcctcttttctacccattttttcctatttatttaaatgtctgtttatttgtctaccatctattatctatctatctgtatctatctatctatctatctatctagtaatcatctatacctatccaacaactgtacatttatttgtttttttttgcatttgctgtttgaaaaaaaatgcaactttttaaaaggcaaagtttaatttatgtaattagatattttcatttttatgaatcatttttaactctaagaaattattaactggcttttctgtggccttctaaaatatcttacaggagagaaagccaaatcacacacatctctctttagtttaaaaattcaataaataagaaagtgagagaagtaatttattatgtactattttgtgatattataatgggtaataattgataagtgtacatttaaatttgtccttgactgaaacagctcctatttcagtcaaggtcaaatattttttattatttctgaaaaaagatagatcataaaaatgccaaaatatactatgagtcatatgatatggggcaatatgtcactggagtaatcgcaaaaggattttctgaagaaagctaaaattatgtaatttgaggtatggatcagttatatattgtaatagcaatgctgtgtatcaaaccaccaaaaaccctgggctctaagctgcttttctagttttgactcctatttccttctgtgtaactcacagactttcttgtcactaagttttacttgtatcattgtttctctattcttacagcttcattttctacatatgtctcttatatatccttcaagatctagtctcaaatccatttcctccataaagctcagaaattaaagtttaccagaaaactctcataatactttgttttgtgataattgttgctttccataactatagaattgtagacaaattgccccaacttaaaatgtacattctttgaggacaaggctatgttttacatgttatagtattacaatttgttctatgcaattttttgacaatagtagatactcaataagtatttgttgaagagcctttgatctagcaatccagaaattatacaaaggtgtttattggattgttattgataatggccagatttaaagcaaacgaagtattcaataatggtggaattgggctgggcacagtggctcacacctgtaatcccagcactttgggaggccgaggcaggcggatcacttgaggtcaggagttcaagaccagccaggcaaacatggtaagaccccatctctaccgggcgtagtggtatgtgcctgtaataccagctacttgggaggctgaggcaggagaatcgcttgaacctgggagacagaggctgcagtgcatgagccgaggtcaggccactgcactccagcccggacaatagagtgagactccctctcaaaaaaaaaaaaaaaaggtggaatagttatattaattatagtaatcatatttagagaaatattatgaaatctttcacaaatttatttacttataataaagatgggaaatagttataccattaagtgaactaatcagaattcaaatatgtaaagtgtccatatagagtggaattacactcataggataaggacaggatggaaataccaacttttggtaagtttattttctttttggttcttctattttttatatattgtgtttttgtaatgtaatccattatagtagtgctataaacataaaaataaatatttattaaacaaatgattaaaaagccatatagatgattttaagatagcttttgtaagcggaagctatcttaaaaattaatgttatttacaatgtattatcaggtaataatgtaaatgaatctcccaccaacacaaatatacctaatcaaagagtaattttttgtcttcatttttttcccacatattttagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggtaggtgatgcatgatccactccttcacctttcatctgaaatcttttccctttcccttcaatcaactcatattacccacttttaaattaaggtgtttgtaagaatgagaagaaatatgtgtgacgtgtttagcacatatgagaggcttagtaaatagcaatttttgtcactctgtctggagtagccctcgggtggaaccaaactcagatcattatggtttcttataatgtttaaagaaggatctttctgactttcagtcatcagaggcagttcttattaagactggttatgtagacatgatgtaggattatcagctaaatatcagactgaagcacgatatttccctgacccctttgcaggtgagaactagagtgcatgggtgccggtaggagcgaactccactcactcactgctccacccctcacaggagggggagcgcaggtgactgggtgcaggagccaaggcaaatgcatttgggcactgcaagagtgaactccataccggccccacaggagcgtctaggggagggtgcctgcgatccttgaagccctagaggaagtgttacagtgcccttttagctttgccatccatggatggcttaaatgttaacagttcagtggagggtcagagtgacagccttttgcacccacacttgtggtacccaagttcatgtccggcgtccaggaggaatgagtttgtacaaatgacttgaagatggtaaatacaggggattttattgccagcgaaagtggctctcagagggaagaggagctgaaaggagatggagcaggaaggtaatcttcccctggagtctggccatccccagccagactcctctccgaagctatgctgtcaagctgtccctctgatgtcaagctacttctctctaatgtccaactgtagtctctgatgtccagctgttcctcctgtctgcctgctgagttctgggctttatataggcacaggatgggggcagggtgcaccatgggtggttttggaaaaggcaacatttaagtgagaaaacagggatgtatattctcactttgggccacggttccaggcttgagggtggagccctcgccaggtacccgtcctcttctgcccagaatttctctgcctcttgttcctgtcaaaattgcttaacataaactccatgctgcaggggactcctctgtcttcttcacactgattcgctattgccaaccacagtgaatgataagaagtagactcacttaattactgactagcaaaaaaatgatggcattacaaacttatgtctgatttcattcaatgaaatgatcaactggatcaaaatattaatataatgaaaatgatatgacctattttcttaattggtgatacaaatgtggttgcattccttttactgtttcaatttaattaataactagagtgtttggtgagttgatttcattaggagaattactgcattggatctggaggcctctaaggcgaattctgatttgactaagaatcctgtgtcctgccatatactcagtttaaagaggatcagccatgctttattttctttacctttattattattattattattttttagacagtcttgctgttgcccaggctggagtgcagtggtgtgatctcgactcactgcagcctccatctcttgggttcatgccattcttgtgccttaacctcgcaagtagctgggattacaggtgtgagctaccacacctggctaatttttgtacttttagtaatagagactgggttttgccatcttggccaggctggtctcgaactcctggcctcaagagatctgcccgtcttggcctcttaaagtgctggaacgacgggcgtgaaccaccgcaccttgccagacatgctttctaaagccaagtagagagagaactatgaagtctcattagtgactagtacctttgctgtaggagctctttgttctcagttacacccagtcagtgctcaccaaattgcacaacgtgctggcacagtggctggctcctcaggggtttacagcttcagctataagcaaagcccagaaacctttaggtccttgtatggagctctggttacaagccctgattcttgttatctaaaaaagaaaatgttcctttgtctttaatccaggctgccaggttttcctgataatttttccgataagaagatcaagttagataaatagtcttttcattctggaagcctcaggagttcctgcaaatgagttacccactctttcccaagggctctggaaaattctgtcaaagggaatttccaaacgtacacccacccgcctccacacacacacagacacacagagagagggagagagacaagaaagtgagcaatgacaatcctttcctttttctgtaggctgagggacctccctgctttatatctgcattactagaggatgcattccattgagtctgcactgaatgagaccaatctactcccaggcgttccactgcctcctgatgtagagagaagcagctggcagtctctcaaaaattttaagctctttgggggtacactgagaccaaaatttaaaaattactgaaacccttggttgactgaaatgcccagtcagcagtcatttatgatcagataatgataaagtaaaattcagccatgggaaacattaaaccttccagccttaggcacctgataagagcttgcatcgtttccttttttaagaaatcatcaattagagactgtttctgatcataaaatttaatagaattttttgacttacaggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtgagtatgctgcactttgctgctttattcattggcatatatgtaatagttctagcaatggtgcctgacacagtgtaggcactcagtaacactgtatcagcccaaatataaattatgtttctcatttcacagtgagaggatgcctcaaaacattttttaccaatttaaatacatatacattcatagataaaaatcaaatgccatcatactatacttattcacttaatttcaaattaatatttaaaatctcaagttatgcaaaataaaatatgaatttagaaattttgctttttgcacactcacatttcgcaaaataacttgtatttaaatttttcacaggcatctttgacattagtatgtttgtcatcactaaagcctgttgagtttaggtcacacagatgaatcattaattacaaagaaatttgaaagtccaaaaagcaagagacaccacttgatttgtatgatatagaagcaaattggctattgaccaagtagccaaagattttattaaaccacattggtgttgaaataaaataagatagagtactaaaatatgagggtttttatataattgaatatgaggcaaatctaccattaaatgtactactactattaaatgtataaaggttacatgcagaattacattaacagtctctggcaataaaggaagacaataaataatatttagaactacataagtgtggacattacaaacaatagaaaatgcaccaaaactataaccattcttttatttgtataatgggattatgcatgatactatttcttttctctattttctgtatgtacttatcataggttggtaaatccataataaaaatatctgatacttgatatatctatgttaggataaaagtatcaagtcagcactgcttgaatataaggaaactcttcagagaaatctagttgtcctgcagctaatgatcatattacccaaagtactctgatatttacctttttagatttaagaaaactattatgatagtatatgaaactgatcaacactttgccttaaatcaaatatgcttattgctcatctatttcattatgaaagatacaaatataaataagtcatttttctagtcctgcagtagcttacagttgaaaagtgaggacagctgcgtacacagtaagtcgacacctgtattacaagtgccacctctttacttgaggaaggaggaaaggcttcaatagggaagtggagtgtgagctggagcttgagagatgtgaatgctagcaggcacagctgagggaggaacacggattcgttaaaacgttggtgcatgacatgcagggcgggttccagaaacaagtagatagggtgaggtaagcctttgtaatgggatgataaggtaagaaagataagttagaaaagatctgaagaacctgagatgccatccaaggaaatttggacttattatttaatacagaggaagctattgaagaattacatatagggaagtgacaagacctgcttgttcttttagtgagggaagttaggtggaggtgagaatgacggaatagaaaggagatttatttagagatcaaaacaccaattaggagattgctgcaatgtcccagaaagagaaggcctatatgtatcttcttttccacatttagctacacaagtcacataaaactgaatattttacaacttcttttcagccagtaaatactaccccattcaaaatattttcctctgtctaacttttatctttcatcctttaacttatgcttatctctttttggttctgtcttcagagaaggtaaagtactacaggtccttatatcttaaatacagaaaagcttcacaactcatgataattcagtaactatttttcaattatctgttaaaaagggacttacaaagcctaagagtttggattttaagggaactatatgaactatgtaagacataattttacaactcattgttttctgtattcaagaggcttcactttcaaattgcatgtgcaaaattattttgaataagttgttttttgtaacaactttcaatgtgcttcacttattttccttaaaaaatatatttttcaaatatattaacaccatactcttaaaagctgtattgcatatttatttttatttatctgcttttgaaattcaggtgtactttagaacaaaatagcttatataattttaataatttttctatatgttttcaaggaaattggacatgtgtatgtcccccgaccgtttttctttttctttttagctaagactttataatttttctcaactacattagtcaactgtatgaataactaaagacaacattgttcttgcaatttctaatttatcataaaatctcaactttttttattcactaattttgtctgacctaattaatgatattatgcccttcaaactgaaatttacaaaagtcaaagctgctttttagaggcctattcctttttaaatgtgttcatgctcatattcaccagtggtttgtatagtttacttgtgtatcaaatgttactttccatttcagatctgctcaatattattagaaatgatacagaaataagttttacagatctgtagaggaagatcacatttctctctcttttttttctttacttttaattttttaaaaacatttcctaccaagaatcttgaaaaagagcacatatatgggcttcttttttataagtgttcgcagactagtatcattaacttcaccctgggaacctgtagaaatgcaaattcttaggcctttccccaaacttactaagtcagactctgctattggtgtttttaacaagacccctgggtgattttgaaactcatgaaagttcgagaattactgattcattgcatagagcaaggctgaactgtgtagacatttttatatgtaaataagaaaattgtgttgctttttctgtataggtctcactggttagcttaactgcaaatgccttgggttactcagaacttggtgccatcaaatccctcagaacactaagagctctgaggccactgagagctttgtcccggtttgaaggaatgagggtaagactgaatgccttagagtttgtcagaattattattgagagcagactgacactttgtaccatggaaatgtcaaatttatggagaatttgtgtcttacacattcatactgacatagctaatcaatcaaaaataatatttaccagatgcccataatacttggcactgctggagtcactcacagagtagtatattgccagagggattgtttctgattagctagattttcacttcttggaaaatctctatagttatgctgctgatttgaatcaagattatttatgttcacttcatttataaatgtgcaggaaatcctactcgctgtagtttaagcctaccaaatcattgctcatcatttcttcactactccgctgtgatacactttgagccttttgatgtttgaatcaggccttttagttcttaaacacaggctgaaatggctaaaaagtaggtcaactggaaatctaacgctcatttagaagggtggtacaaaagaacagaggagtttgtgctgacatttgtcgtcccctgaggcacaaaacctgagaccacataccctcaccacctagaaaatgatgatgccttgtctcagttgttttagctggttcaaagaggattttaaaaaaatgatactttttgtgatatttgaaaataagttgcttagactttatctgcatgttatagtgatactagctcatattttctaactaagaaaatagttacttagactttatctagtgttacaatcacaactagagatgaatggtgtgtgtagatgtgtgtctgtatatgcatggttacatagaaaagtgttattagcggtaaaattctttttactttaccaattagaaagaacagtttttgcagtagaaggcttaataaacaaaaggtatcaatctttcagtaccagaatactgtttatattttctgtgtggaatttgatccccaagtggtctcttttactctcaaattttggacagcaaattgtatggtttgtatgattttttgaaagtgatgttcacttctatattcatgccactgtttatactcttaattatttttggcatttgctgttagttccatcctttgaggtaaatttgctacatgtgtgttattacctcttgagaaaacattctccaatataaaattcgttgtatactcttctgatttataattttaaaattcttagttggagctaccagagtctagtttctacccaatattcaactttgaaacagatttttttaatcatttgactgttcttttaataatgtttaaaaataagtaaatatttgttgttggcttttcacttatttttccttctcatcctgtgccaggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagtaagtaatcactttattattttccatgatgtgtaattaaaatgagtctaaagtttttcttcctcataatgagatatccacctgttagaatggctattatcaaacagataaatgacaataaatgctggcaagaatgtgaagaaaagggaacccttgtacattgttggcagggatgtaaattagtatagcttttatggaaaacagtatggaggtttctcaaaaaactaaaaatagaaccactatgtgatccaacaattccattactgggtatatatacaaaggaaattaaatcaacatgtcaaagagatgtctgcactctcacactcactgcagcactattcacaatagccaaaatatggaaacaacctaattgtccatcaacagatatgtggataaagaaaagtgtgtgtgtgtgtgtgtgtgtgtacatatatgtatatgtatatatatacacacacgtatttctatatacacacgtatagatatacactgtatatgtatatatctatacacatatatagacatacacagaaacagtgtttgtgtatgtgtgcgtgtatatagaagtagtcagggaaggggcagagcctgtggcactaagaaactgagaaaatgtacaagacttttgttttcagaattactatgtccgcacaacagaaaaagtatttcaaaaagtaaatgcgcttgaatgtatttgttttcagtttaggaaactgcttctttttgtagagtgccttaaaatagtatgttcaacaatattaaaaagattttcaaaaataagccctcgtgattgatgattggtaataatcatttaaaaacttattggatgtatatatatgtgtgtgtatacacgcacacacacacacacacacccctatagacatacacaatgaaatagtattcagcctttaaagaagaaggaaatcctgtccttttatacaacattgattcacctggaggaaattaagtgaaataagccaggcacagaaagacaaatgacacatgatgtcacttatatatggaatctaaaaaacacaaactcacagaaacagaaagcagaatgacaatcaccaggggctgggggatgatgggagatgttggtcaaaggatacaaaattcaattcgacaggaagaatacattctgtagagctattgtacagcatggtgactatagttaataataatatattatatacttgaaaatagctaagtgagtagatatgttttctcatcagaaaaaaataagtataagaagcgataattatatattacttagcttgagttagccatttcacaatatatatatatttgaaaacatcattttgtacaacataaatatattcatttttatttgtcaattaaaaaatgaatatatttttgaaaagcaattaaaataaaaatgcatatacattttaggaactctatatagatgcactaaaactatataaaaatgatataatactatacaacaataaaataaaatttttcttcctctgtgtttacaaatacttccttaggcccatctgcctagattcctcttaccatgattgaactatcttttctgccccacgctggaaacatgatggttctaaaaactttattgtctccctgactatgcatttggtagcatagccaagtcctttgttactgggagtttaatctaggcactcattgttttcctcccttcctactctgaggaaagaagtgctggccccaaggggggttgaaaaggggtgtgtgtgtgtgtgtgtatgtgtccacacgcgtgtgtgtagatagagaaagagagagagactttcaaataggaaaattgctctcttgcaaatgaaaactttccaattaagactattgtgtctgctatgcactcataataattcattcagctattcaactgactgcagtattaaatctccactagctcctggacacaatccacttacacgatcctcaagactattaaaatagtcaggaaaggggaagagcctgtggcactaaggaactgaaaaaatgtacaagagttttattttcaagatcattatgtcaacggagcagaaaacaaatatttaaaaaaggaaatgcagtagaatatattgttttcagattaggaaactgcttcctcttatagagtaatcacctcaaaatagtatgatcaacaatattaagaagattttcaagaataagctgtcatgattggtgattggtgtaataatcatttagaaaagaataagtagaaaggaagcattaagataaataatgcagcatacttttgagcttgtctcatgctgctactatacacatgaaattttttcatcaaagttcatgatatatttttatataaacacatcagagtcaaagattgttcatattgtttttatgatagcatattgttacagtagatcattatttaattatatatgctaaatatccacataagatgttatagaggaatataaatttgaagtattttcaatgcatatcgcaaaacattgccccaaaagtgaatacaaatttcaagcttatttatatgcctgtattgaatacatgtcaaatagaattttgatcaattattcaatttattttctaaaattataattttgggaaaaaagaaaatgatatgacttttcttacaggccacgtttaagggatggatggatattatgtatgcagctgttgattcacgaaatgtaagtctagttagagggaaattgtttagtttgattaaatgtatatttctacaatattgtaatttagtgatattgtcaataaaataaaattatgtgcttaatttataaaacccatctatattataaggataaaatatttaatcatactatttctttcaaaattatcataggatgattttctctaatcactctgtatcttttaacatatcttttctagtatttagcaaggcacctgacacaaaactttattgtatgtattttcaaaatgagacattttatttttggctctgatagtcctggtcatttgtgcattagaagttctcacaggcaatattttttatctgtaatatatttcctccagcttttgatcttccttataataggaaggatatgactaaaaacggggacaaaaataaacaatttagtgtttctcttgggaaagtgagattaagtggtagaagggagggacttccctaatctactttatacataccagtactttgaattcttttctataattttcattaatttctcactatttaatgaggaatgaagtcacattttgaaaaaaaaaaaaaaagagattgatttctggtatgccagagcatgataataaagctcaaaatgctctttccctagcaccagcagctagctttctgagtgaagaattcctgaggtttttttttttcttttttccacttcataaaaacagagagggagcaagaaagcatgaaaagccctgcattgtatctctataagtgctatcaggaattccagttatgagatttttctgaatagtaataataatttattgattatcactattcactgtgccaaggactttctcacattatcccatttaatcctaaatgacaaccttattgtataggtgatactagctctattttactactgaagcaaagaggcttaatgcgttaaatgggaaaacaagtttttgaaccctgaccacaaataatggctcatacccactttccacagtggttcttacctttttgattaattaattcaatgctctctccaccttccttatcaatagcttatatgccatgaaacattttcagtttcttctttaataacttagcagaccttttccgctgcaaaactcctggaatttccagcacattacaaaagatgaaagccaattgagcactacatttatgaaaagttgctggatcttgaactttaattagtaaattgcatcagataaatgcaaatttaaaccaaaataaaacattatctacacacctaccagattggcaataccaaaaagtctgacaataccaagttttaccaaggataaacagcaataagaacactcgtacaatgctgataggaaaaaaaatagttaaataatcctttaaaaacagttgggtatgatcacattatttgagaaagttaaagatattttttaatactgcaattctactttgaacaacgtatcctaaagaaacttatgcacatgtttaggataatctatgtacaaaaatgaatataacttttttttgcacttgcaaaaaactgggagcaactcaaaaacagtagaaataggcaaataattgaatactatatagtgatgaaaatgaatgaataccgccatatacaaccacatggatgagccttaaaaatacaacattgagttaaagaaactagacacatactataattctacttatataaagttcgaaattgacaaaactaagcttattgttcaaaactgcatactgaggtgttaacttgaaagaaaaagcagggacatcattaccataaaagtcaggataatgattacctccagcagggatgatggagtttatgtttgagaagggtacaccaagggtttctgaagttgtagcaatgtcctgggttatggatttcacttataaaacatattatattttgcatttatgtattatgcactttcctgtatgtatattgtcttttaaaaattttaaaaatataattttacatcactgttaactaaactcacatacacaaataaaatctcatcgaagaatagcagttttacaatattcctgatattttccattttgctgtatttccttagaaacaaaattatgctggtcataatcctctaaattgatttcataacacagtgggttataacttgcatctattatcatcatcagggattggttaactgagttggttagaacaatgtcctattagacctgtgaaagcttacagctaaggcgcaaacctactatcacacagttttctaaacaaaagtggattagacaagagatagtatcattgttacagaaacagtccctactgaataggataaagcaatagattcattttcagaaaggaaagatcaacctatatacctacatgcagacctactacaatgattcttgcctatctaaagaaatgtattataccaaacccttacacttagcaattactactggccgccactgttctaagcatatttatatgttaatatagttaatcttcacaaccacactatgaggtttaagtttgattattttcatctcacagatgagaaaactgagtcagagaaagtaaatcttaaaagttttgacatagaataatgtgacgctgacatctcttttgtaagaagaggaaatctttaatttgcatgctgtgttgggaactttgcttagaaaggaaagtgcattcataatctgggcatttgttgggtgaaattgtctataatcattcagacttctatatggttatttcattttcccaggtaatgaatagtcttgcagaactcttcaataagcatgtgagatttgaaggttcataaaatctgtttagtgtttggtttattttcattccagagattaaaacatgcttagataattaaaaactcactgatgtactttttgtgaaacaagtactagatataatggttacaattcttcatattctttaggtagaattacaacccaagtatgaagacaacctgtacatgtatctttattttgtcatctttattatttttggttcattctttaccttgaatcttttcattggtgtcatcatagataacttcaaccaacagaaaaagaagataagtatattaaaacttcatccttgctctgaaatatgaactaaatatttcatactctttcctttagcctccaaaatgcaatcaccaaaaaaagaatataaaattcagaaattattttgagacatttgataatcgataagcttttaagcaattaataattcagatagcatgtttttgatatttttagtctagaaatatgactaatatggcataatttatatattgaataaaggcatctctataaatacagatattagtaacaatagaatgaaatgtgggagccaattttcacatgattactaaggtggattttatagccagcaaagaacacaattttaacaagtgttgctttcatttctttactttggaggtcaagacatttttatgacagaagaacagaagaaatactacaatgcaatgaaaaaactgggttcaaagaaaccacaaaaacccatacctcgacctgctgtaagaataacatattttcattgcctgttaaaactatattacctaaccgtttcacagcccgaatttctagaaactagttatttttgtggatttgtaacacaaagttttttaccttaacaatgggactagctagcctaaatagcttgaaaaatgtactttacatatataatatgtataaattatataatgcataacatattttatatgtaaacatataaaatacatagaaataaaatttgctatacttaagtgccagtggtatcatacaagctgatgtcattaagacacttctaataacatcaaaaataaaatacatacatacataatgtgaaaatattaaatgttctcagagtacagaggagacagatcggaataattggtacgtcacagattggcctcagtttttgtccaactctgcagattgaatggaatcattaatgaaacaggccacaggttttgcttttttctggttaaacaaaaaaaagacaaacctcatattttcccctactatcccacccttaaatgagatgatatcattctttgtagggctttttattggctcttccaggtgtacatttgccagtgatactgttcgttcagtttggctgctgcagggagttgctgccaggagaatcgctaagtttttctatcactcctgaaggactagctcatatattaagtctcagaaaatcttccccaacgtatacgtggtataaaacacttcagtgtttctcagaaatcttgactctataaatctattggtgacaatataaaacagaccgtaattaagtgttcagttggtaagccggccaataactcaaagaaaatggatagctatattgggtcaaacacaaagggtgtacaacttgagcctagtctttaggaaataatacaatttgaatgaatagagagagaagcagagaacatttactgtatgagaaaatgtatacttcatagccatatagacaaatatatcagtgcagaatagtgatgcatttgaattagtgagtagtagacactggttttccgagttacatgagacaaggttaccatacgagtctgaagaaatttgttctaattaagcaatacaaatgcaatatagttaacagaacagcctagtaatgtgaaaagaaagattttagagagtttaacctagagactggtgtggaacaatattagaggcaaaataaccctcggccatagacaagaagataaacccttacatacaagaagatagtccataatctgtgtccaaccagcaggactggaactactccaggagtgaagttagccaataagaagactcaattgggatgaaacacaggaaaagagggaggatgcaatgaaaaaactgggttcaaagaaaccacaataacccatacctcgacctgctgtaagaatagcatattttcattgcctgttatgaaacacaggaaaagagggaggatatgtaaataacagagaatctaaaatataagctagttgatattttgtgaaactgttggttccactatcatatactgaagtcatatgaaggcactgggaaaaatagtgttagagcctatgaaatgtccagactgaaataaggattttagcattgtcagaacaaaattcaattgagctctgaaacacagattcatttttgaaaaataattagaatagagaaaaaaacaaaattctcagaatgaggccttgcatacttcatcaagatataggaagaaataaatcaatgaagaaatgagcttgagtttgtttccatcaaatgacatggatttacctgtagtggtaggggtgtgtggaaaaagttcaacacattcagctagaatattatcagtgtcaatttggcaatttagcaagtaactagtaaaatccatttattcctgcattgacaatatgtactatgtagtatgctaagcatttgaacttaaatatcgaacagtatggagtctagttaatgcaacggatagtaatcaaatagtcctgccaaaaaatggaagtatcccagaaaaaaagggatactttcagctgtgagagctgattagggggaaggggctgattaatcagggaagttagggaaggctttattaaaaaaatatactagctgaggatggaaaaagaatagaaagcatcaatagccagagtgggatgagaagagccctgtagaaggggaatgaatttgtgaaggtccttatgtaggagggctggtgagactggagtgcagaaagtcaaggttcatttgggacacactgagaataaagaggttaggataagcccaaacttttctgggccttggaggccgtgttaaggagtagttttcatcctaagagcagtaagaaaccgttaacgtggacccagtcagtctgggctttgtggtgatcactcaatcagtttcacagaggccgtgtgaatacattgtagacttgttttggagctatttcagagatggtaggtagcctgaaccatagcaatgtgcagattaataaaagtggatggatttgtgagctatcaccagagtgaaatttaaaagtttgtctattaattgaatatgggaactaaagaaggaaccaacaagaatgactggtgtctttctgctttgcacaactggataaatactgatgtcatgcaggaaatgaagaagggacagaaagtggtgagaaaattggagatgctagtttgcagaatttggcaaacgagtcagagtgagagagtgagaggaaggaggaagggagagaaatgatgaatatttagaagtagcaaaataaaggtttcttaagattcagagattaggtttaaaggaaagcaaaaggaattttagagaggaaaagatcgaagacagagggaataattacggcataaaaatgcacaagatgtgggacaaggacatagtggtctagggtagctttagaaagaaaaaggggctgagtcctctaatgaatttggagtaatatatgaaaagaacatggaaattaaaataatatgaaatgcaaaaggaaacagaggagtttatttaaactgtttaaatttaatgttctaaaaaaagtaaaaatagagggcaagagaatggaaatttatgagaagtttggaattatctttggagcaaatgaaggacaaaggattgctaattgttaaatctgaagggccaagatgaagttagagaacataaatttttggtgaataagatcttcagaattatacatcttgttccagcatatttgacaccctaggatttaaatgggagaacagaacacagagactcgagactggagttgtacattgagatgtctgtctcattggacaactctatgaacagggaatctaaacagttttttattagtcatggtgatattaaaattaagaccaaatttctgcttttaagatattttgaacttactatactctaggagccatatctgagagaaaaatgatactgctcctgcttttgaggggcctcaaaacaagtggaagggaaagaaaactaaaattgaataagagcaaaccatttgcaatacaatgccatacattttatgatcaatgaaagcactcagttttctgagagcactaagtgctttaaactcaaaactgagttagaattcatgagacagagaaggagtgggggacatgtgttttagacagaatagtagacagataaactatgtaaaatgatacagtagaacctgcctaagcttctaagagtggtaggcaggaaatatcagagggtggaagtaaggggaagatgccagacttggaaagttaaactacagtaaattaataattaataaggaagggtttgaactaaaagtagacacatttattgggttgaaaaaggccctgagaagacagatccagctggagtaatagaaatctagttcagcaggccaaatacgcattcagagaaaagagcaaaacaaaatagaacaagtagtgtttgatgtccaaaaatcaccattggaagtaatagaagcctcccaatagaaagagggcagaacactaagatgtagaatccaggccactaaaagtgtcaggatctgggaaagcaagccattaggtgtatatgtagcagagtattagtcattctagttgagaaggtagagaaaggcagcccaacagaggttaagtcaagaccagatccctagattacctgagaaacaaagcagatacgtgcaaaatggaacaatacagaaaccaatgatcagaactggtttacaagttggggacttcatttcataagcaagacataaggcaattagtacttggaaataaggtccaaatagactagggcaaagattgaatatttccattgtgactttttaaaagataattttattcttacagaagagttactcataatgaatactctaatgaatctatacacagtgtcctcttgttttaacatcttatgcaaccatagatcagttctcacaactaagaaattaatcttgatataataccattaaataaaatacagaactgagtcagatttcaccagtttttccactgaagccttttctctagaatgatgatttttaaaacatcttagctgaactttaaaatgaaatttaagatgctgtagctttagtgagagaatataaagtcagaaatcagacgaaaaatttaaaaagagagaggaaaacttggagaagtatttatttattagttgcttaaagtaaaattaataccctcccaacacatgggataaaaaattttattacatgacaaatatttactaactgtccgtcataacatgatggtgttctgtgcactgagaacataatacgtgagtttataaaacctggtatcaatgtgagtataaataaaacaaatacatttgaatacagttgaatatacaatatacaaaattttcttccaagtataaaacgaaaataaaatacactactttctttaatagaatagaacattgtaataatgttccattgcatttgaccctcacataaatgctatgaggtagcattaagagataagatttgaggctgggcatggtggctcatgcctgtaattccagcactttgggaggtcgaggtgggcagatcattaggtcaggagtttgagaccagcctgaccaatacggtgaaatcccgtctctactaaaattacaaaaagtagtcgggcatggtggcatgtacctgtaatcccagctactcaggaggctgaggcaggagtatcgcttgaacccgggaggcagaggttgcagtgagccaagatcgtgccactgcactccaacctgtgcaacagagcgagactccatctcaaaaaataaaaaaaattaaaaaaaagagagagagataagatttgagatctgacatggagcttccctatttacactacttacctgctttgtgacctaaggcaagttacctcagctctccaatcactggttttgcaaggaatttttttttttgtaaaatgttgtgaggattaaagatgtgtttttataaaagctacattttttgttgctttcttaaaatcagaagaattgaattcgattttttttaaggtttctaatggaacttttacatattatttgttccagaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggtaagaagaggtgcttttattcagttaaggaatatagtggtaaaaatatgtgttttaaaactttagaggtgtttttcactaatctttctcattcatcccaaactcccaaataaaaatctaatagtccattgttttagttttagtttgccatttctctaattgcatgctgtgcttgaaatgatgagtggaatacaaggaatttatattttcagctttcatttattctcatttaatattttcatctgttctcatctcagaagacaataactgcaactttggtagaatagtcttgtacctggtcatactcctgtggtattgacagttactgctttgaataaacaatcaatccacacacatatatacataaatcatttgaagtagtcacataattcataaatatgacctcttaaataattggaatagtgtatatgtgcagttatatatataataacacatatataagtttcatgttatctttgggtgcagacagttttctgtggtttgcaatatctctttttggaagcagatagtttgtttgaaaatccaaaacagatttgttatcatcaatgatacattaatgttaggatacatacatacattaagtcctaggaatgcaaaagatttattggaaaaaatatatatatacagtgtttatgtataagatattaaatgaggtactggaagtaaatataagaagatttaagagaaggttctacctatttggggaaacagaacattcacatggaggggaaaattatatagcactctttaaactactttctttagtcgaatagaacattgtaacaatgttccactgcatttgattctcacataagtgctatgaggtagcattaagaggtaagatttgagatctgacctggagcttccctatttacactacttaccttctcagtgacctaagagaagttacctcagctctccaatctctggttttgcaaggaatttttctgtaaaatgttattgtgaggattaaatcagattatgtatatatatgcacttagcactgtgcctagcatgaagaaaagacttagtaaatgttcagtttgaccacaagaaaaagttgatattatcaccatttactcatgcataaaagcaagtgccaggattcagtcccaagtacatctgtctccaaagcctatgttttcttctgtacatcacgctgcctactcccaaataacatagaatctcagaaagtaaagaactctcatattcctgacccaaaatcatacacctttagttcttatgcaaatactagaactagtattttggacatataaattaatttctgtacttggccactgtatgcttcatgatgtctttggaccttccagggttgagtcatttttttgatagatgctttccttgaactaggaaaaatggcccttattatcttcatttaatataaagatgtaaatgttataacaccaaacataccagtttcattttgctcaacaaacattgcagattatttgcatatatacatgtacctaactgtcctgttcacattttgtaaaactaatgtacttatgtaaactttcatttgctactattaagtataacaatatttttgttatttgttgattttctacaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaacattttaagttgtgcatcttttctccttcctgtccacctattgttctttttttcacttttccatttcaatttcttccttatgtattcttgatctacttttctttatatccttctatcctttccttgcgctctcagtatttttcatttaggatattctccttgtttcttttctgttcaccaaatgtcttgtttattacagcctatagatcacttagatttagatccctaaaatttgctgtcactctgtaaagtgcacatagtatctgcgtatttatttttacaattttctttacaaaaattctaagaaagcttagactgcaacttttcataatatataaacaaaattccaaaatagcacagatttgactttctcatttaattatgaacagtttgggaaatgtttctcaattctgaatgcatattagaactacctgagaagcaaaaaaaaaaaaaaaaaaaaagccagtgcatgggccaagaccaggaccagaccaattgaactcatatctctgggtgagacttaagcatttaattttttgaaggctcgtcattgatactgattcataccaaggactgagaactagcaaatttgggtctcctttgcccaataggaaaacatattctccggaaagtccaaactgtccttcattacataacttatcatgtacagaataaagttcctaagaggatggctttcggagtatttatattgcagattgagggattgacattgatctaattataatagaggttcttctttagggctgtagttggtataaagggaattctattagaaaagtttagggagactgtgttcaggtaacacatgcacacacacacaccccatatggcaccttaactatccctagtgtatactgggtggaatgaatgtgggatggtctaggaaacagtataaatttataaaatagttccctccagtgaatgctacatgccctgttgcatgagaacctttaatctggaccaatagaaggcagttcatggtggccagtcagaaagccattcaatttgtcattaccaactcacaaatctagacaggaaagattctcattctcatcttctccattcttggcgcatctcacacaatcccttgtttttctgagctctgtaacatacagacctatttacaaacagaagggaggagacaagagaggtaaggggaagtaaagaaagaggagaaaggaggagagggaacgataggggaaagtaggagaaaggaggggagtgtaagaaatgggatgtttcatgaattataattctgaaaaaatgtctggtgtggatacctccagcaatggtgagggaaggaggaagaaatgttgcttagttgtcaatatgttagatctaaaatataaacatgtttcttctcttatttatttttatttgtatttatttatttatttatttttgagataaggtcttgctctgtcacccaggctgaagagcagtggtgtgatcatggctcactgcagccttaaccttctggactcaagtgatcttcccacctcagcctcccgagtatctgggactacaggcatgtgctcccacacccagctcatttttttttttttttttgtattttttgtagagacagggtctccccatgttgcctgggttggtctcagatttctggactgaagcaatcatcctgcctcagctttccaaagtgctagaattacaggcatgagccattgcacctgcccctctcatttttttaaagaaaagatacctgtgaaaaaagtgaagaggacattatacaagaaaatgttacaatgattccatggattcaggaatatttttttttcttttacctgaactaaatacatatttgagtgtctactatatatcaactttgcaatagcagctaagagatcaaagaggaatataactatccaaagaaaggctataaccagaggtgaagaacctggaaagccatgctagagtttggaattgattctgtggcaaaaaggagtgattactcctgagaagtaga

SEQ ID NO: 3 - protein, 2005 aa, Homo sapiens
MAQSVLVPPGPDSFRFFTRESLAAIEQRIAEEKAKRPKQERKDEDDENGPKPNSDLEAGKSLPFIYGDIPPEMVSVPLEDLDPYYINKKTFIVLNKGKAISRFSATPALYILTPFNPIRKLAIKILVHSLFNMLIMCTILTNCVFMTMSNPPDWTKNVEYTFTGIYTFESLIKILARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCLQWPPDNSSFEINITSFFNNSLDGNGTTFNRTVSIFNWDEYIEDKSHFYFLEGQNDALLCGNSSDAGQCPEGYICVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMLEQLKKQQEEAQAAAAAASAESRDFSGAGGIGVFSESSSVASKLSSKSEKELKNRRKKKKQKEQSGEEEKNDRVRKSESEDSIRRKGFRFSLEGSRLTYEKRFSSPHQSLLSIRGSLFSPRRNSRASLFSFRGRAKDIGSENDFADDEHSTFEDNDSRRDSLFVPHRHGERRHSNVSQASRASRVLPILPMNGKMHSAVDCNGVVSLVGGPSTLTSAGQLLPEGTTTETEIRKRRSSSYHVSMDLLEDPTSRQRAMSIASILTNTMEELEESRQKCPPCWYKFANMCLIWDCCKPWLKVKHLVNLVVMDPFVDLAITICIVLNTLFMAMEHYPMTEQFSSVLSVGNLVFTGIFTAEMFLKIIAMDPYYYFQEGWNIFDGFIVSLSLMELGLANVEGLSVLRSFRLLRVFKLAKSWPTLNMLIKIIGNSVGALGNLTLVLAIIVFIFAVVGMQLFGKSYKECVCKISNDCELPRWHMHDFFHSFLIVFRVLCGEWIETMWDCMEVAGQTMCLTVFMMVMVIGNLVVLNLFLALLLSSFSSDNLAATDDDNEMNNLQIAVGRMQKGIDFVKRKIREFIQKAFVRKQKALDEIKPLEDLNNKKDSCISNHTTIEIGKDLNYLKDGNGTTSGIGSSVEKYVVDESDYMSFINNPSLTVTVPIAVGESDFENLNTEEFSSESDMEESKEKLNATSSSEGSTVDIGAPAEGEQPEVEPEESLEPEACFTEDCVRKFKCCQISIEEGKGKLWWNLRKTCYKIVEHNWFETFIVFMILLSSGALAFEDIYIEQRKTIKTMLEYADKVFTYIFILEMLLKWVAYGFQVYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINYTTGEMFDVSVVNNYSECKALIESNQTARWKNVKVNFDNVGLGYLSLLQVATFKGWMDIMYAAVDSRNVELQPKYEDNLYMYLYFVIFIIFGSFFTLNLFIGVIIDNFNQQKKKFGGQDIFMTEEQKKYYNAMKKLGSKKPQKPIPRPANKFQGMVFDFVTKQVFDISIMILICLNMVTMMVETDDQSQEMTNILYWINLVFIVLFTGECVLKLISLRYYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSGPPDCDPDKDHPGSSVKGDCGNPSVGIFFFVSYIIISFLVVVNMYIAVILENFSVATEESAEPLSEDDFEMFYEVWEKFDPDATQFIEFAKLSDFADALDPPLLIAKPNKVQLIAMDLPMVSGDRIHCLDILFAFTKRVLGESGEMDALRIQMEERFMASNPSKVSYEPITTTLKRKQEEVSAIIIQRAYRRYLLKQKVKKVSSIYKKDKGKECDGTPIKEDTLIDKLNENSTPEKTDMTPSTTSPPSYDSVTKPEKEKFEKDKSEKEDKGKDIRESKK

SEQ ID NO: 4 - DNA, 8895 bp, Homo sapiens
aacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcaggggtttttctccctttgcttgacacttctctgtcctgacaccttgagaagaaggatgtgtttgcttacccttccgccatgattgtaaatttcctgaggccttcccagccatgcagcactcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcgtatgtaacagaatttgtaaacctaggcaatgtttcagctcttcgaactttcagagtcttgagagctttgaaaactatttctgtaattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

SEQ ID NO: 5 - protein, 2005 aa, Homo sapiens
MAQSVLVPPGPDSFRFFTRESLAAIEQRIAEEKAKRPKQERKDEDDENGPKPNSDLEAGKSLPFIYGDIPPEMVSVPLEDLDPYYINKKTFIVLNKGKAISRFSATPALYILTPFNPIRKLAIKILVHSLFNMLIMCTILTNCVFMTMSNPPDWTKNVEYTFTGIYTFESLIKILARGFCLEDFTFLRDPWNWLDFTVITFAYVTEFVNLGNVSALRTFRVLRALKTISVIPGLKTIVGALIQSVKKLSDVMILTVFCLSVFALIGLQLFMGNLRNKCLQWPPDNSSFEINITSFFNNSLDGNGTTFNRTVSIFNWDEYIEDKSHFYFLEGQNDALLCGNSSDAGQCPEGYICVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMLEQLKKQQEEAQAAAAAASAESRDFSGAGGIGVFSESSSVASKLSSKSEKELKNRRKKKKQKEQSGEEEKNDRVRKSESEDSIRRKGFRFSLEGSRLTYEKRFSSPHQSLLSIRGSLFSPRRNSRASLFSFRGRAKDIGSENDFADDEHSTFEDNDSRRDSLFVPHRHGERRHSNVSQASRASRVLPILPMNGKMHSAVDCNGVVSLVGGPSTLTSAGQLLPEGTTTETEIRKRRSSSYHVSMDLLEDPTSRQRAMSIASILTNTMEELEESRQKCPPCWYKFANMCLIWDCCKPWLKVKHLVNLVVMDPFVDLAITICIVLNTLFMAMEHYPMTEQFSSVLSVGNLVFTGIFTAEMFLKIIAMDPYYYFQEGWNIFDGFIVSLSLMELGLANVEGLSVLRSFRLLRVFKLAKSWPTLNMLIKIIGNSVGALGNLTLVLAIIVFIFAVVGMQLFGKSYKECVCKISNDCELPRWHMHDFFHSFLIVFRVLCGEWIETMWDCMEVAGQTMCLTVFMMVMVIGNLVVLNLFLALLLSSFSSDNLAATDDDNEMNNLQIAVGRMQKGIDFVKRKIREFIQKAFVRKQKALDEIKPLEDLNNKKDSCISNHTTIEIGKDLNYLKDGNGTTSGIGSSVEKYVVDESDYMSFINNPSLTVTVPIAVGESDFENLNTEEFSSESDMEESKEKLNATSSSEGSTVDIGAPAEGEQPEVEPEESLEPEACFTEDCVRKFKCCQISIEEGKGKLWWNLRKTCYKIVEHNWFETFIVFMILLSSGALAFEDIYIEQRKTIKTMLEYADKVFTYIFILEMLLKWVAYGFQVYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINYTTGEMFDVSVVNNYSECKALIESNQTARWKNVKVNFDNVGLGYLSLLQVATFKGWMDIMYAAVDSRNVELQPKYEDNLYMYLYFVIFIIFGSFFTLNLFIGVIIDNFNQQKKKFGGQDIFMTEEQKKYYNAMKKLGSKKPQKPIPRPANKFQGMVFDFVTKQVFDISIMILICLNMVTMMVETDDQSQEMTNILYWINLVFIVLFTGECVLKLISLRYYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSGPPDCDPDKDHPGSSVKGDCGNPSVGIFFFVSYIIISFLVVVNMYIAVILENFSVATEESAEPLSEDDFEMFYEVWEKFDPDATQFIEFAKLSDFADALDPPLLIAKPNKVQLIAMDLPMVSGDRIHCLDILFAFTKRVLGESGEMDALRIQMEERFMASNPSKVSYEPITTTLKRKQEEVSAIIIQRAYRRYLLKQKVKKVSSIYKKDKGKECDGTPIKEDTLIDKLNENSTPEKTDMTPSTTSPPSYDSVTKPEKEKFEKDKSEKEDKGKDIRESKK

SEQ ID NO: 6 - DNA, 8776 bp, Homo sapiens
aacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcagcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcgtatgtaacagaatttgtaaacctaggcaatgtttcagctcttcgaactttcagagtcttgagagctttgaaaactatttctgtaattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

SEQ ID NO: 7 - protein, 2005 aa, Homo sapiens; identical to SEQ ID NO: 5

SEQ ID NO: 8 - DNA, 8604 bp, Homo sapiens
atagcagtaacacaattcacctctagtgtgaacatatcaggatggcatagaccagcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcatatgtgacagagtttgtggacctgggcaatgtctcagcgttgagaacattcagagttctccgagcattgaaaacaatttcagtcattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

SEQ ID NO: 9 - protein, 2005 aa, Homo sapiens; identical to SEQ ID NO: 3

SEQ ID NO: 10 - DNA, 8630 bp, Homo sapiens
aagcatgatggaattttagctgcagtcttcttggtgccagcttatcaatcccaaactctgggtgtaaaagattctacagggcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcatatgtgacagagtttgtggacctgggcaatgtctcagcgttgagaacattcagagttctccgagcattgaaaacaatttcagtcattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

SEQ ID NO: 11 - protein, 2005 aa, Homo sapiens; identical to SEQ ID NO: 3

SEQ ID NO: 12 - DNA, 926 bp, Homo sapiens
gtgagttcttagtcaagttgccttcactgcctatttactaattggttctgggctagtcccagggatgatggtgaagaaggctggcctccttccctctgtctaaagtatcactaagatgctggatgggcctgaccgtgtaatggaccaatgatcctagaagtcttttggaagcactcatttgaacctgcatttgtgagacaggcagagaactggtgaggcatcctccagcgcgggaattaaggaaggacaaaagcctattcaccttcttgaatacaaattatatgcttaaaccagtgtaaattgaccctgattccctaataatgttgagaagcaaaaactgtaaactaggagtctatttaaattttattttttatatttgcaggagtagtatctaaattcctctttatagtctctagctctccataagtcactttgatcttcagtgggtttaattattcctttataccatactttctcctttctattgctctccacagaaggaataatagcaggtgacttgtaggtgccaaataagattctgagcaaagaacacacctggaaaaccttgaagttctcatgagaaaattttctaaccaaaaaaaaaaatcaaagcctcaattttgtgctttatgtgaattataaatgcggttttaaaatacttacattaaaacttgataaagttgctaagaattcctatggcattgatcacaaattttcttaataatcctcatgtcatttatcaaatttaggaaagtttatagtgctcagaaaaaaaaagcatctatcttcatgtcatatgatggtaattattatgttatacactattttacagggcaatatttataaataatggttttacttttctcttaaaatattcttaatatatattctaagttttattttatgtgttgtgttttctttttcag

SEQ ID NO: 13 - DNA, 27 bp, Homo sapiens
ctttatttcagcactttcttatgcaag

SEQ ID NO: 14 - DNA, 20 bp, Homo sapiens
tcttctgcaatgcgttgttc

SEQ ID NO: 15 - DNA, 20 bp, Homo sapiens
tttttccctccctgtttctg

SEQ ID NO: 16 - DNA, 20 bp, Homo sapiens
tcttctgcaatgcgttgttc

SEQ ID NO: 17 - DNA, 27 bp, Homo sapiens
gacccctactatatcaataagaaaacg

SEQ ID NO: 18 - DNA, 22 bp, Homo sapiens
agggttgaagggagttaaaatg

SEQ ID NO: 19 - DNA, 26 bp, Homo sapiens
agaaagtgagttcttagtcaagttgc

SEQ ID NO: 20 - DNA, 20 bp, Homo sapiens
tgcaggttcaaatgagtgct

SEQ ID NO: 21 - DNA, 29 bp, Homo sapiens
ggtacattctttattcaatatgctcatta

SEQ ID NO: 22 - DNA, 20 bp, Homo sapiens
tctaaacaaaagccccttgc

SEQ ID NO: 23 - DNA, 26 bp, Homo sapiens
ggtacattcatatcctttttcaaatc

SEQ ID NO: 24 - DNA, 20 bp, Homo sapiens
cacttagaagacgggggaaa

SEQ ID NO: 25 - DNA, 20 bp, Homo sapiens
atatcgtagggggaccaacc

SEQ ID NO: 26 - DNA, 20 bp, Homo sapiens
tctaaacaaaagccccttgc

SEQ ID NO: 27 - DNA, 25 bp, Homo sapiens
cacagtcattacttttgcatatgtg

SEQ ID NO: 28 - DNA, 23 bp, Homo sapiens
tgtctgaaattgttttcaatgct

SEQ ID NO: 29 - DNA, 22 bp, Homo sapiens
aaggtggtacgccccttatatc

SEQ ID NO: 30 - DNA, 20 bp, Homo sapiens
ttgttttcaatgctcggaga

SEQ ID NO: 31 - DNA, 21 bp, Homo sapiens
attccagacctgaagaccatt

SEQ ID NO: 32 - DNA, 20 bp, Homo sapiens
gaggccattggaaacattta

SEQ ID NO: 33 - DNA, 20 bp, Homo sapiens
ttctccgagcattgaaaaca

SEQ ID NO: 34 - DNA, 21 bp, Homo sapiens
gcaaagaatcaaatccatcca

SEQ ID NO: 35 - DNA, 20 bp, Homo sapiens
cctgaagaccattgtggggg

SEQ ID NO: 36 - DNA, 18 bp, Homo sapiens
aaagtgacttttatcctc

SEQ ID NO: 37 - DNA, 20 bp, Homo sapiens
tgtttgcaatggcctccaga

SEQ ID NO: 38 - DNA, 25 bp, Homo sapiens
agtctaagtcacttgattcacatct

SEQ ID NO: 39 - DNA, 19 bp, Homo sapiens
agatgcacgccagtgtcct

SEQ ID NO: 40 - DNA, 23 bp, Homo sapiens
cagttgataaaggttttcccaga

SEQ ID NO: 41 - DNA, 20 bp, Homo sapiens
tagaggggcaaaatgatgct

SEQ ID NO: 42 - DNA, 20 bp, Homo sapiens
tggagggttttgagtgagga

SEQ ID NO: 43 - DNA, 24 bp, Homo sapiens
ctttatcaactgacactacgtgct

SEQ ID NO: 44 - DNA, 20 bp, Homo sapiens
ccttctgttcagcctcttcc

SEQ ID NO: 45 - DNA, 20 bp, Homo sapiens
accccaactatggctacacg

SEQ ID NO: 46 - DNA, 22 bp, Homo sapiens
gcaattctcttgagttcggtgt

SEQ ID NO: 47 - DNA, 19 bp, Homo sapiens
caagaagaagctcaggcgg

SEQ ID NO: 48 - DNA, 21 bp, Homo sapiens
cgattttcggactctgtcatt

SEQ ID NO: 49 - DNA, 20 bp, Homo sapiens
aggtttccgtttttccttgg

SEQ ID NO: 50 - DNA, 20 bp, Homo sapiens
aaggcggttttctctccatt

SEQ ID NO: 51 - DNA, 23 bp, Homo sapiens
ctccacaccagtccttactgagc

SEQ ID NO: 52 - DNA, 20 bp, Homo sapiens
ttgtcctcaaaggtgctgtg

SEQ ID NO: 53 - DNA, 20 bp, Homo sapiens
aggtttccgtttttccttgg

SEQ ID NO: 54 - DNA, 20 bp, Homo sapiens
aaggcggttttctctccatt

SEQ ID NO: 55 - DNA, 22 bp, Homo sapiens
ctaccagagggcacaactactg

SEQ ID NO: 56 - DNA, 20 bp, Homo sapiens
cattgctctttgccttgatg

SEQ ID NO: 57 - DNA, 20 bp, Homo sapiens
gactgcaatggtgtggtctc

SEQ ID NO: 58 - DNA, 20 bp, Homo sapiens
tgggcaacaaactcaacaaa

SEQ ID NO: 59 - DNA, 23 bp, Homo sapiens
catggaagaacttgaagaatcca

SEQ ID NO: 60 - DNA, 20 bp, Homo sapiens
tgggatagtgctccatagcc

SEQ ID NO: 61 - DNA, 20 bp, Homo sapiens
ttttgaccaacaccatggaa

SEQ ID NO: 62 - DNA, 20 bp, Homo sapiens
gcagctttctggaggctaaa

SEQ ID NO: 63 - DNA, 20 bp, Homo sapiens
tggaaacctggtcttcacag

SEQ ID NO: 64 - DNA, 19 bp, Homo sapiens
cggaatgatcggagaactg

SEQ ID NO: 65 - DNA, 20 bp, Homo sapiens
ccatcaccatctgcattgtc

SEQ ID NO: 66 - DNA, 22 bp, Homo sapiens
gggatgctaaacatttgatgtc

SEQ ID NO: 67 - DNA, 19 bp, Homo sapiens
ggctgctccgagttttcaa

SEQ ID NO: 68 - DNA, 20 bp, Homo sapiens
acacgatcaggaaggagtgg

SEQ ID NO: 69 - DNA, 20 bp, Homo sapiens
ggcaaatgtggaaggattgt

SEQ ID NO: 70 - DNA, 20 bp, Homo sapiens
tggtggtatggcaatcgaat

SEQ ID NO: 71 - DNA, 24 bp, Homo sapiens
ggaaatctagtgcttctgaacctc

SEQ ID NO: 72 - DNA, 22 bp, Homo sapiens
gctttctgcttcctaacaaagg

SEQ ID NO: 73 - DNA, 20 bp, Homo sapiens
gccaaaccatgtgccttact

SEQ ID NO: 74 - DNA, 21 bp, Homo sapiens
gacaataggaagtggccttga

SEQ ID NO: 75 - DNA, 22 bp, Homo sapiens
gcaaagagaagctaaatgcaac

SEQ ID NO: 76 - DNA, 20 bp, Homo sapiens
gccttcaggttcaagggatt

SEQ ID NO: 77 - DNA, 20 bp, Homo sapiens
ccaaatgctgttggagaatc

SEQ ID NO: 78 - DNA, 20 bp, Homo sapiens
acaggaaggaaacacgcaat

SEQ ID NO: 79 - DNA, 25 bp, Homo sapiens
ctgttttacagaagactgtgtacgg

SEQ ID NO: 80 - DNA, 22 bp, Homo sapiens
gctcagcagaatgatgaagaca

SEQ ID NO: 81 - DNA, 20 bp, Homo sapiens
tgaggaatcccttgaacctg

SEQ ID NO: 82 - DNA, 21 bp, Homo sapiens
ttcaaacagcaaatgcaaaaa

SEQ ID NO: 83 - DNA, 21 bp, Homo sapiens
gggctctcgcctttgaagata

SEQ ID NO: 84 - DNA, 20 bp, Homo sapiens
aggaagtctagccagcacca

SEQ ID NO: 85 - DNA, 20 bp, Homo sapiens
caaagggaaactctggtgga

SEQ ID NO: 86 - DNA, 20 bp, Homo sapiens
ttgaagggaaagggaaaaga

SEQ ID NO: 87 - DNA, 22 bp, Homo sapiens
ctgattgttgatgtctcactgg

SEQ ID NO: 88 - DNA, 19 bp, Homo sapiens
attccttcaaaccgggaca

SEQ ID NO: 89 - DNA, 20 bp, Homo sapiens
atgctgctaaagtgggttgc

SEQ ID NO: 90 - DNA, 20 bp, Homo sapiens
tgtcaggcaccattgctaga

SEQ ID NO: 91 - DNA, 22 bp, Homo sapiens
gaatgagggttgttgtaaatgc

SEQ ID NO: 92 - DNA, 20 bp, Homo sapiens
acctggcagtttgattgctc

SEQ ID NO: 93 - DNA, 20 bp, Homo sapiens
aggccactgagagctttgtc

SEQ ID NO: 94 - DNA, 20 bp, Homo sapiens
gactccagcagtgccaagta

SEQ ID NO: 95 - DNA, 20 bp, Homo sapiens
gtagccacgtttaagggatg

SEQ ID NO: 96 - DNA, 23 bp, Homo sapiens
gacaccaatgaaaagattcaagg

SEQ ID NO: 97 - DNA, 20 bp, Homo sapiens
gccaggtggaaaaatgtgaa

SEQ ID NO: 98 - DNA, 20 bp, Homo sapiens
ttacatccctgccaacaatg

SEQ ID NO: 99 - DNA, 23 bp, Homo sapiens
cacgaaatgtagaattacaaccc

SEQ ID NO: 100 - DNA, 23 bp, Homo sapiens
gacaccaatgaaaagattcaagg

SEQ ID NO: 101 - DNA, 20 bp, Homo sapiens
cacgtttaagggatggatgg

SEQ ID NO: 102 - DNA, 28 bp, Homo sapiens
cagagtgattagagaaaatcatgctatg

SEQ ID NO: 103 - DNA, 21 bp, Homo sapiens
ggtgtcatcattttggaggtc

SEQ ID NO: 104 - DNA, 20 bp, Homo sapiens
cgaggtatgggtttttgtgg

SEQ ID NO: 105 - DNA, 22 bp, Homo sapiens
caacccaagtatgaagacaacc

SEQ ID NO: 106 - DNA, 20 bp, Homo sapiens
tttggtgattggattttgga

SEQ ID NO: 107 - DNA, 22 bp, Homo sapiens
cgacctgctaacaaattccaag

SEQ ID NO: 108 - DNA, 21 bp, Homo sapiens
ttcagcacacattctccagtg

SEQ ID NO: 109 - DNA, 20 bp, Homo sapiens
aacccatacctcgacctgct

SEQ ID NO: 110 - DNA, 21 bp, Homo sapiens
aggctaggtagtcccattgtt

SEQ ID NO: 111 - DNA, 21 bp, Homo sapiens
cattgtaggaatgtttctggc

SEQ ID NO: 112 - DNA, 20 bp, Homo sapiens
acatcccaaagatggcgtag

SEQ ID NO: 113 - DNA, 20 bp, Homo sapiens
tgtggtggtcattctctcca

SEQ ID NO: 114 - DNA, 21 bp, Homo sapiens
ctcatcatttcaagcacagca

SEQ ID NO: 115 - DNA, 18 bp, Homo sapiens
ctaagaactcactttctt

SEQ ID NO: 116 - DNA, 18 bp, Homo sapiens
actaagaactcactttct

SEQ ID NO: 117 - DNA, 18 bp, Homo sapiens
gactaagaactcactttc

SEQ ID NO: 118 - DNA, 18 bp, Homo sapiens
tgactaagaactcacttt

SEQ ID NO: 119 - DNA, 18 bp, Homo sapiens
ttgactaagaactcactt

SEQ ID NO: 120 - DNA, 18 bp, Homo sapiens
cttgactaagaactcact

SEQ ID NO: 121 - DNA, 18 bp, Homo sapiens
acttgactaagaactcac

SEQ ID NO: 122 - DNA, 18 bp, Homo sapiens
aacttgactaagaactca

SEQ ID NO: 123 - DNA, 18 bp, Homo sapiens
caacttgactaagaactc

SEQ ID NO: 124 - DNA, 18 bp, Homo sapiens
gcaacttgactaagaact

SEQ ID NO: 125 - DNA, 18 bp, Homo sapiens
ggcaacttgactaagaac

SEQ ID NO: 126 - DNA, 18 bp, Homo sapiens
aggcaacttgactaagaa

SEQ ID NO: 127 - DNA, 18 bp, Homo sapiens
gaaggcaacttgactaag

SEQ ID NO: 128 - DNA, 18 bp, Homo sapiens
tgaaggcaacttgactaa

SEQ ID NO: 129 - DNA, 18 bp, Homo sapiens
gtgaaggcaacttgacta

SEQ ID NO: 130 - DNA, 18 bp, Homo sapiens
agtgaaggcaacttgact

SEQ ID NO: 131 - DNA, 18 bp, Homo sapiens
cagtgaaggcaacttgac

SEQ ID NO: 132 - DNA, 18 bp, Homo sapiens
gcagtgaaggcaacttga

SEQ ID NO: 133 - DNA, 18 bp, Homo sapiens
ggcagtgaaggcaacttg

SEQ ID NO: 134 - DNA, 18 bp, Homo sapiens
aggcagtgaaggcaactt

SEQ ID NO: 135 - DNA, 18 bp, Homo sapiens
taggcagtgaaggcaact

SEQ ID NO: 136 - DNA, 18 bp, Homo sapiens
ataggcagtgaaggcaac

SEQ ID NO: 137 - DNA, 18 bp, Homo sapiens
aataggcagtgaaggcaa

SEQ ID NO: 138 - DNA, 18 bp, Homo sapiens
aaataggcagtgaaggca

SEQ ID NO: 139 - DNA, 18 bp, Homo sapiens
taaataggcagtgaaggc

SEQ ID NO: 140 - DNA, 18 bp, Homo sapiens
gtaaataggcagtgaagg

SEQ ID NO: 141 - DNA, 18 bp, Homo sapiens
agtaaataggcagtgaag

SEQ ID NO: 142 - DNA, 18 bp, Homo sapiens
cccagaaccaattagtaa

SEQ ID NO: 143 - DNA, 18 bp, Homo sapiens
ctgaaaaagaaaacacaa

SEQ ID NO: 144 - DNA, 18 bp, Homo sapiens
tgaaaaagaaaacacaac

SEQ ID NO: 145 - DNA, 18 bp, Homo sapiens
gaaaaagaaaacacaaca

SEQ ID NO: 146 - DNA, 18 bp, Homo sapiens
aaaaagaaaacacaacac

SEQ ID NO: 147 - DNA, 18 bp, Homo sapiens
aaaagaaaacacaacaca

SEQ ID NO: 148 - DNA, 18 bp, Homo sapiens
aaagaaaacacaacacat

SEQ ID NO: 149 - DNA, 18 bp, Homo sapiens
aagaaaacacaacacata

SEQ ID NO: 150 - DNA, 18 bp, Homo sapiens
agaaaacacaacacataa

SEQ ID NO: 151 - DNA, 18 bp, Homo sapiens
gaaaacacaacacataaa

SEQ ID NO: 152 - DNA, 18 bp, Homo sapiens
aaaacacaacacataaaa

SEQ ID NO: 153 - DNA, 18 bp, Homo sapiens
aaacacaacacataaaat

SEQ ID NO: 154 - DNA, 18 bp, Homo sapiens
aacacaacacataaaata

SEQ ID NO: 155 - DNA, 18 bp, Homo sapiens
acacaacacataaaataa

SEQ ID NO: 156 - DNA, 18 bp, Homo sapiens
cacaacacataaaataaa

SEQ ID NO: 157 - DNA, 18 bp, Homo sapiens
acaacacataaaataaaa

SEQ ID NO: 158 - DNA, 18 bp, Homo sapiens
acataaaataaaacttag

SEQ ID NO: 159 - DNA, 18 bp, Homo sapiens
cataaaataaaacttaga

SEQ ID NO: 160 - DNA, 18 bp, Homo sapiens
aaacttagaatatatatt

SEQ ID NO: 161 - DNA, 18 bp, Homo sapiens
aacttagaatatatatta

SEQ ID NO: 162 - DNA, 18 bp, Homo sapiens
cttagaatatatattaag

SEQ ID NO: 163 - DNA, 18 bp, Homo sapiens
ttagaatatatattaaga

SEQ ID NO: 164 - DNA, 18 bp, Homo sapiens
tagaatatatattaagaa

SEQ ID NO: 165 - DNA, 18 bp, Homo sapiens
agaatatatattaagaat

SEQ ID NO: 166 - DNA, 18 bp, Homo sapiens
tattaagaatattttaag

SEQ ID NO: 167 - DNA, 18 bp, Homo sapiens
ttaagaatattttaagag

SEQ ID NO: 168 - DNA, 18 bp, Homo sapiens
taagaatattttaagaga

SEQ ID NO: 169 - DNA, 18 bp, Homo sapiens
aagaatattttaagagaa

SEQ ID NO: 170 - DNA, 18 bp, Homo sapiens
agaatattttaagagaaa

SEQ ID NO: 171 - DNA, 18 bp, Homo sapiens
taagagaaaagtaaaacc

SEQ ID NO: 172 - DNA, 18 bp, Homo sapiens
aagagaaaagtaaaacca

SEQ ID NO: 173 - DNA, 18 bp, Homo sapiens
agagaaaagtaaaaccat

SEQ ID NO: 174 - DNA, 18 bp, Homo sapiens
gagaaaagtaaaaccatt

SEQ ID NO: 175 - DNA, 18 bp, Homo sapiens
aaaagtaaaaccattatt

SEQ ID NO: 176 - DNA, 18 bp, Homo sapiens
aaagtaaaaccattattt

SEQ ID NO: 177 - DNA, 18 bp, Homo sapiens
aagtaaaaccattattta

SEQ ID NO: 178 - DNA, 18 bp, Homo sapiens
agtaaaaccattatttat

SEQ ID NO: 179 - DNA, 18 bp, Homo sapiens
gtaaaaccattatttata

SEQ ID NO: 180 - DNA, 18 bp, Homo sapiens
taaaaccattatttataa

SEQ ID NO: 181 - DNA, 18 bp, Homo sapiens
aaccattatttataaata

SEQ ID NO: 182 - DNA, 18 bp, Homo sapiens
accattatttataaatat

SEQ ID NO: 183 - DNA, 18 bp, Homo sapiens
ccattatttataaatatt

SEQ ID NO: 184 - DNA, 18 bp, Homo sapiens
ttatttataaatattgcc

SEQ ID NO: 185 - DNA, 18 bp, Homo sapiens
atttataaatattgccct

SEQ ID NO: 186 - DNA, 18 bp, Homo sapiens
tttataaatattgccctg

SEQ ID NO: 187 - DNA, 18 bp, Homo sapiens
ttataaatattgccctgt

SEQ ID NO: 188 - DNA, 18 bp, Homo sapiens
tataaatattgccctgta

SEQ ID NO: 189 - DNA, 18 bp, Homo sapiens
ataaatattgccctgtaa

SEQ ID NO: 190 - DNA, 18 bp, Homo sapiens
taaatattgccctgtaaa

SEQ ID NO: 191 - DNA, 18 bp, Homo sapiens
aaatattgccctgtaaaa

SEQ ID NO: 192 - DNA, 18 bp, Homo sapiens
aatattgccctgtaaaat

SEQ ID NO: 193 - DNA, 18 bp, Homo sapiens
atattgccctgtaaaata

SEQ ID NO: 194 - DNA, 18 bp, Homo sapiens
tattgccctgtaaaatag

SEQ ID NO: 195 - DNA, 18 bp, Homo sapiens
attgccctgtaaaatagt

SEQ ID NO: 196 - DNA, 18 bp, Homo sapiens
ttgccctgtaaaatagtg

SEQ ID NO: 197 - DNA, 18 bp, Homo sapiens
tgccctgtaaaatagtgt

SEQ ID NO: 198 - DNA, 18 bp, Homo sapiens
gccctgtaaaatagtgta

SEQ ID NO: 199 - DNA, 18 bp, Homo sapiens
ccctgtaaaatagtgtat

SEQ ID NO: 200 - DNA, 18 bp, Homo sapiens
cctgtaaaatagtgtata

SEQ ID NO: 201 - DNA, 18 bp, Homo sapiens
ctgtaaaatagtgtataa

SEQ ID NO: 202 - DNA, 18 bp, Homo sapiens
tgtaaaatagtgtataac

SEQ ID NO: 203 - DNA, 18 bp, Homo sapiens
gtaaaatagtgtataaca

SEQ ID NO: 204 - DNA, 18 bp, Homo sapiens
taaaatagtgtataacat

SEQ ID NO: 205 - DNA, 18 bp, Homo sapiens
aaaatagtgtataacata

SEQ ID NO: 206 - DNA, 14 bp, Homo sapiens
agtgccacccctgc

SEQ ID NO: 207 - DNA, 29 bp, Homo sapiens
tctggacccctactatatcaataagaaaa

SEQ ID NO: 208 - DNA, 26 bp, Homo sapiens
gggttgaagggagttaaaatgtaaag